(19) United States
(12) Patent Application Publication
(10) Pub. No.: US 2022/0289698 A1
(43) Pub. Date: Sep. 15, 2022

(54) INHIBITORS OF HUMAN ATGL

(71) Applicants: Karl-Franzens-Universität Graz, Graz (AT); Technische Universität Graz, Graz (AT)

(72) Inventors: Gernot GRABNER, Graz (AT); Rudolf ZECHNER, Graz (AT); Robert ZIMMERMANN, Graz (AT); Rolf BREINBAUER, Graz (AT); Anna MIGGLAUTSCH, Graz (AT); Nikolaus GUTTENBERGER, Graz (AT)

(73) Assignees: Karl-Franzens-Universität Graz, Graz (AT); Technische Universität Graz, Graz (AT)

(21) Appl. No.: 17/631,437

(22) PCT Filed: Jul. 30, 2020
(86) PCT No.: PCT/EP2020/071590

§ 371 Date: Jan. 28, 2022

(30) Foreign Application Priority Data
Jul. 30, 2019 (EP) 19189199.3

(51) Int. Cl. C07D 277/56; C07D 213/55; C07D 417/12; C07D 401/12; C07D 409/12; C07D 417/04; C07D 409/10

(52) U.S. Cl. CPC C07D 277/56; C07D 213/55; C07D 417/12; C07D 401/12; C07D 409/12; C07D 417/04; C07D 409/10; C07D 405/12

(57) ABSTRACT

The present invention relates to novel inhibitors of adipose triglyceride lipase (ATGL) having an improved inhibitory activity against human ATGL (hATGL) as well as pharmaceutical compositions comprising these inhibitors, and their therapeutic use, particularly in the treatment or prevention of a lipid metabolism disorder, including, e.g., obesity, non-alcoholic fatty liver disease, type 2 diabetes, insulin resistance, glucose intolerance, hypertriglyceridemia, metabolic syndrome, cardiac and skeletal muscle steatosis, congenital generalized lipodystrophy, familial partial lipodystrophy, acquired lipodystrophy syndrome, atherosclerosis, or heart failure.

The present invention relates to novel inhibitors of adipose triglyceride lipase (ATGL) having an improved inhibitory activity against human ATGL (hATGL) as well as pharmaceutical compositions comprising these inhibitors, and their therapeutic use, particularly in the treatment or prevention of a lipid metabolism disorder, including, e.g., obesity, non-alcoholic fatty liver disease, type 2 diabetes, insulin resistance, glucose intolerance, hypertriglyceridemia, metabolic syndrome, cardiac and skeletal muscle steatosis, congenital generalized lipodystrophy, familial partial lipodystrophy, acquired lipodystrophy syndrome, atherosclerosis, or heart failure.

Obesity and associated comorbidities have become a major issue for public health. Adipose tissue expansion is often associated with insulin resistance, a hallmark of metabolic and cardiovascular complications of obesity. Upon excess nutrient supply, energy is stored as triacylglycerols (TGs) within adipocytes of adipose tissue. The enzyme Adipose Triglyceride Lipase (ATGL) initiates the degradation of TGs and hence critically determines the availability of free fatty acids and their concentration in the circulation.

Pharmacological inhibition of ATGL using the small molecule inhibitor atglistatin has been reported to protect mice from high fat diet induced metabolic disorders (WO 2014/114649; Mayer N et al., Nat Chem Biol. 2013; 9(12):785-7; Schweiger M et al., Nat Commun. 2017; 8:14859). Atglistatin acts as a locally and timely restricted competitive inhibitor of murine ATGL. The inhibition of ATGL by atglistatin leads to reduced lipid deposition with subsequently decreased adipose tissue mass, TG content, and inflammation. Decreased TG content is also observed in several other tissues including liver, skeletal and cardiac muscle, indicating major differences between pharmacological inhibition and global genetic deletion of ATGL. Additionally, atglistatin treatment has also been shown to lead to improved insulin sensitivity and glucose tolerance in mice.

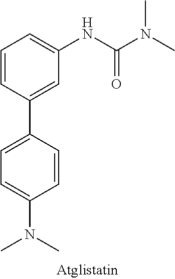 [SMILES: CN(C)C(=O)Nc1cccc(-c2ccc(N(C)C)cc2)c1]

However, while atglistatin is a potent inhibitor of murine ATGL, and as such is a valuable research tool compound, it has poor inhibitory activity against human ATGL (IC₅₀>200 μM). There is hence an unmet need for novel and improved ATGL inhibitors that are active against human ATGL.

Certain 2-phenylthiazole derivatives have been described as potentially useful for the treatment of Alzheimer's disease based on their activity as inhibitors of acetylcholinesterase and/or butyrylcholinesterase (Shi D H et al., Chemistry Select 2017; 2(32):10572-9; CN 106749090). Moreover, the synthesis of various thiazole derivatives has been described in: Liu Y et al., Synthesis 2017; 49(21):4876-86; Hodgetts K J et al., Org Lett. 2002; 4(8):1363-5; and Kim H S et al., J Heterocyclic Chem. 1995; 32(3):937-9. The reference Badr MZA et al., Bull Chem Soc Jpn. 1981; 54(6):1844-7 discloses the synthesis of certain (satured) thiazolidine derivatives but not any (aromatic) thiazole derivatives. WO 2007/042250 and WO 2009/148004 describe specific prolinamide derivatives and specific carbohydrazide-substituted pyridine derivatives, respectively, as well as corresponding synthetic intermediates.

In the context of the present invention, it has surprisingly been found that the compounds of formula (I), as described and defined herein below, are highly effective in inhibiting human ATGL and are therefore particularly well-suited as therapeutic agents for human medicinal use. The present invention thus solves the problem of providing improved ATGL inhibitors targeting human ATGL.

The present invention hence provides a compound of the following formula (I)

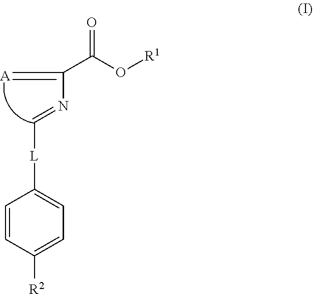

or a pharmaceutically acceptable salt or solvate thereof, wherein A is —CH═C(R^{A1})—CH═ or —S—C(R^{A2})═.

Accordingly, formula (I) embraces compounds containing a pyridine or thiazole ring, which is attached in a specific orientation to a phenyl ring (via the linker group L) and to an ester group —COO(R¹):

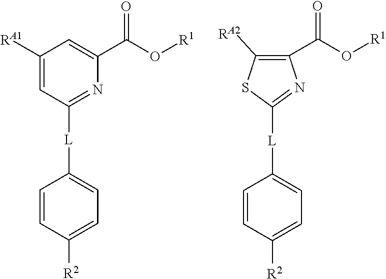

It has been found that the compounds according to the present invention, containing a pyridine or thiazole ring in the specific orientation required in formula (I), have a particularly potent inhibitory activity on human ATGL (hATGL) in comparison to corresponding compounds containing such a ring in a different orientation, and also in comparison to compounds containing other aromatic rings instead. This can be illustrated with reference to the compound of Example 101 (AM-50) according to formula (I), which exerts a potent inhibitory activity on hATGL with an IC₅₀ of 2.5 μM, whereas the reference compound AM-52, containing a thiazole ring in a different orientation, merely has an IC₅₀>200 μM on hATGL, and whereas the reference compounds AMU-27, AMU-4-245 and AM-30, containing other heteroaromatic rings, have considerably lower inhibitory activities on hATGL (with IC₅₀ values of 200 μM or more):

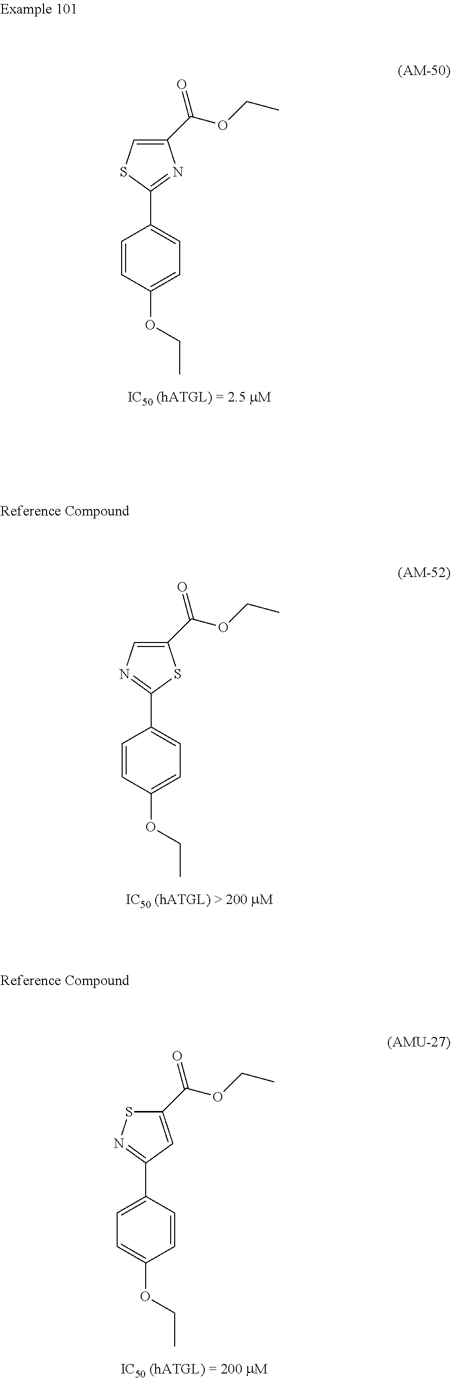 [SMILES: CCOC(=O)c1csc(-c2ccc(OCC)cc2)n1 ; CCOC(=O)c1cnc(-c2ccc(OCC)cc2)s1 ; CCOC(=O)c1cc(-c2ccc(OCC)cc2)ns1]

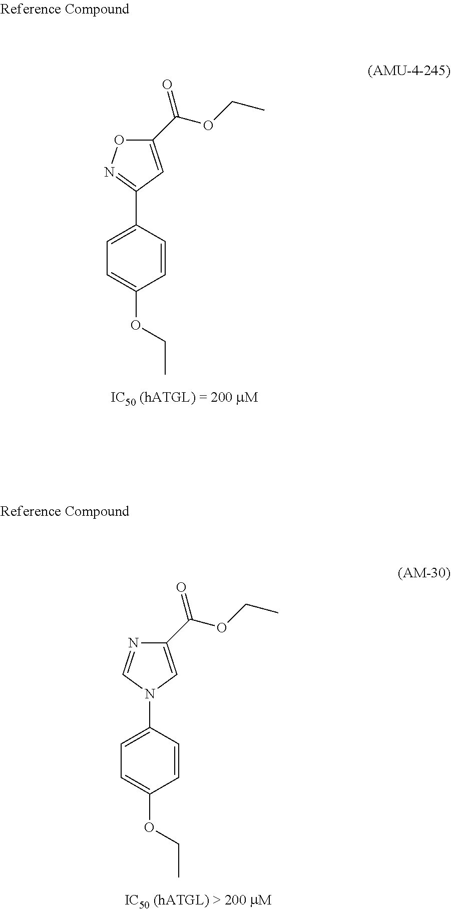 [SMILES: CCOC(=O)c1cc(-c2ccc(OCC)cc2)no1 ; CCOC(=O)c1cn(-c2ccc(OCC)cc2)cn1]

Moreover, it has been found that the presence of an ester group —COO(R¹) in the specific position defined in formula (I) is essential for the potent inhibitory activity of the compounds of formula (I) on human ATGL, as reflected by the following comparative examples:

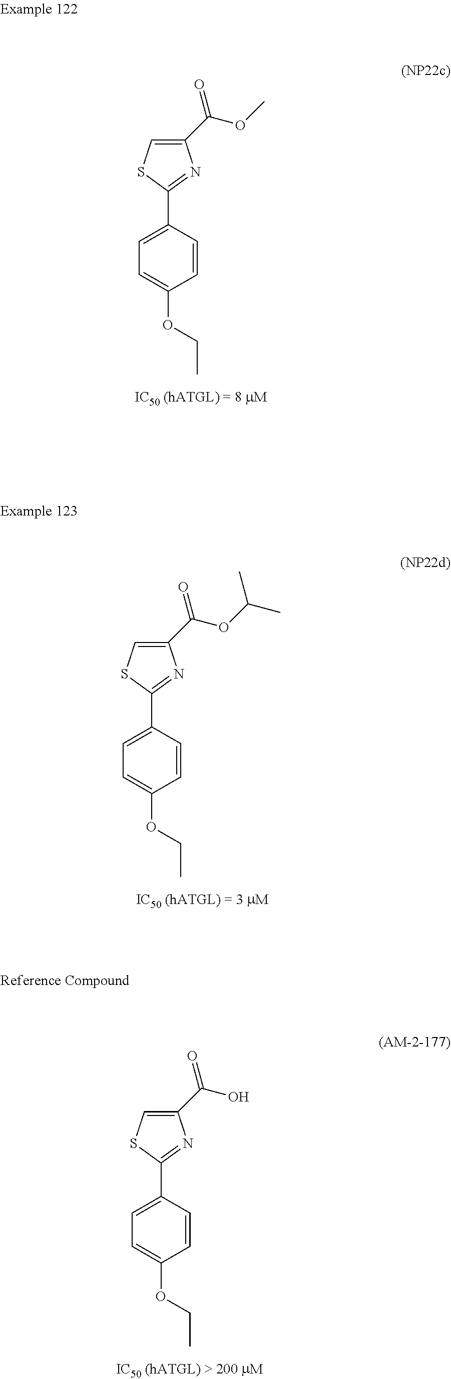 [SMILES: CCOc1ccc(-c2nc(C(=O)OC)cs2)cc1 ; CCOc1ccc(-c2nc(C(=O)OC(C)C)cs2)cc1 ; CCOc1ccc(-c2nc(C(=O)O)cs2)cc1]

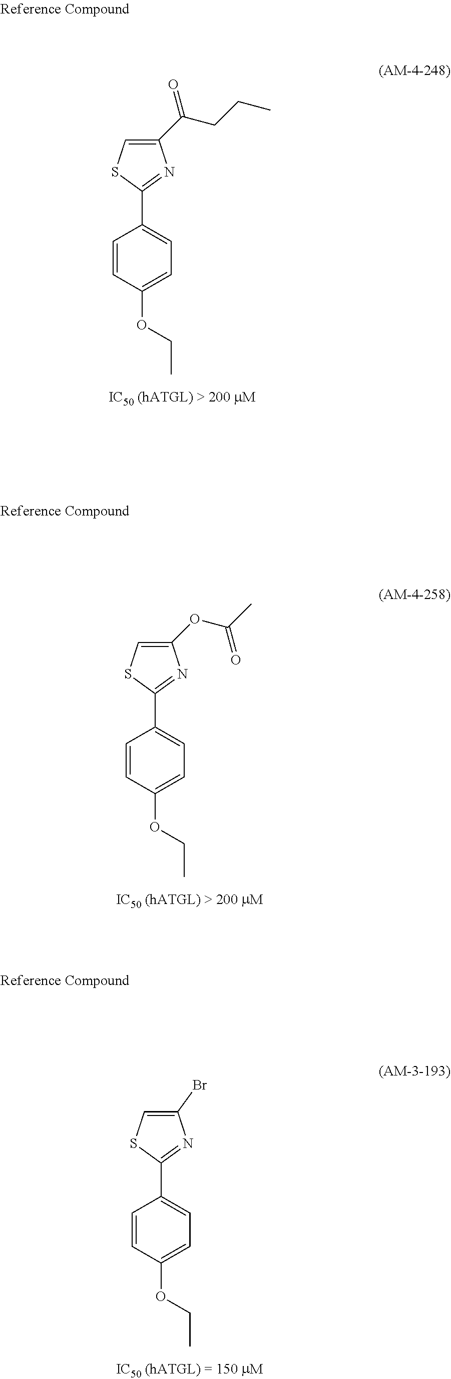 [SMILES: CCCC(=O)c1csc(-c2ccc(OCC)cc2)n1 ; CCOc1ccc(-c2nc(OC(C)=O)cs2)cc1 ; CCOc1ccc(-c2nc(Br)cs2)cc1]

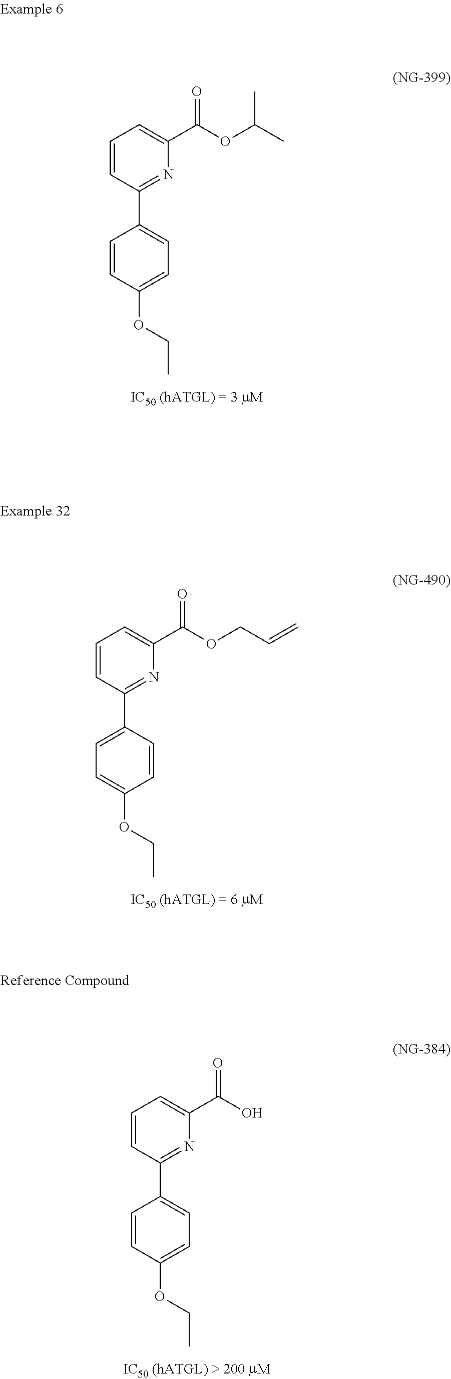 [SMILES: CCOc1ccc(-c2cccc(C(=O)OC(C)C)n2)cc1 ; C=CCOC(=O)c1cccc(-c2ccc(OCC)cc2)n1 ; CCOc1ccc(-c2cccc(C(=O)O)n2)cc1]

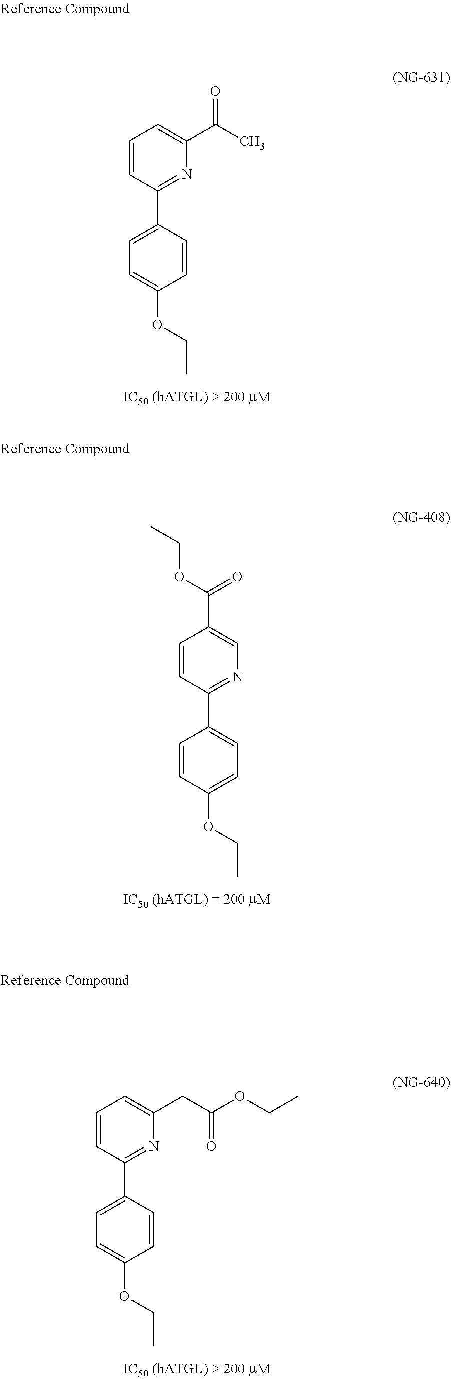 [SMILES: CCOc1ccc(-c2cccc(C(C)=O)n2)cc1 ; CCOC(=O)c1ccc(-c2ccc(OCC)cc2)nc1 ; CCOC(=O)Cc1cccc(-c2ccc(OCC)cc2)n1]

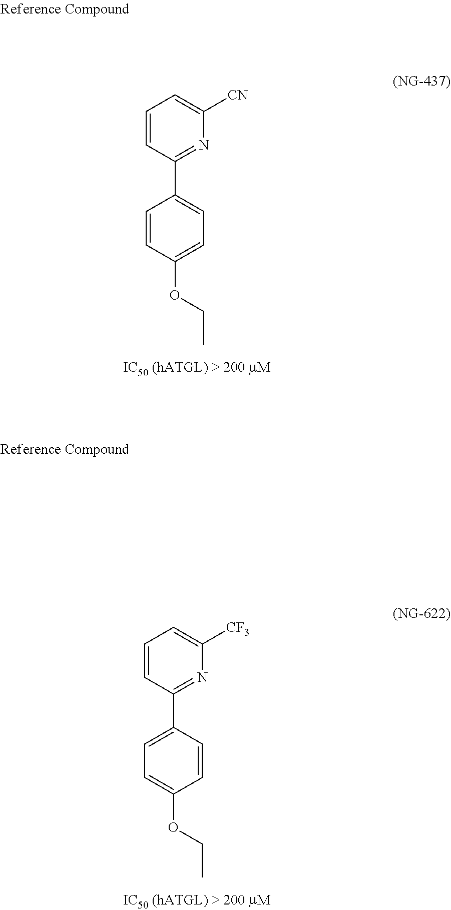 [SMILES: CCOc1ccc(-c2cccc(C#N)n2)cc1 ; CCOc1ccc(-c2cccc(C(F)(F)F)n2)cc1]

The compounds of formula (I) comprise a phenyl ring that carries a substituent R² in para-position (with respect to the pyridine or thiazole ring) but which is unsubstituted in the ortho- and meta-positions. It has been found that compounds of formula (I) having hydrogen atoms in the ortho- and meta-positions of the phenyl ring exhibit a particularly advantageous inhibitory activity on human ATGL, as illustrated by the following examples:

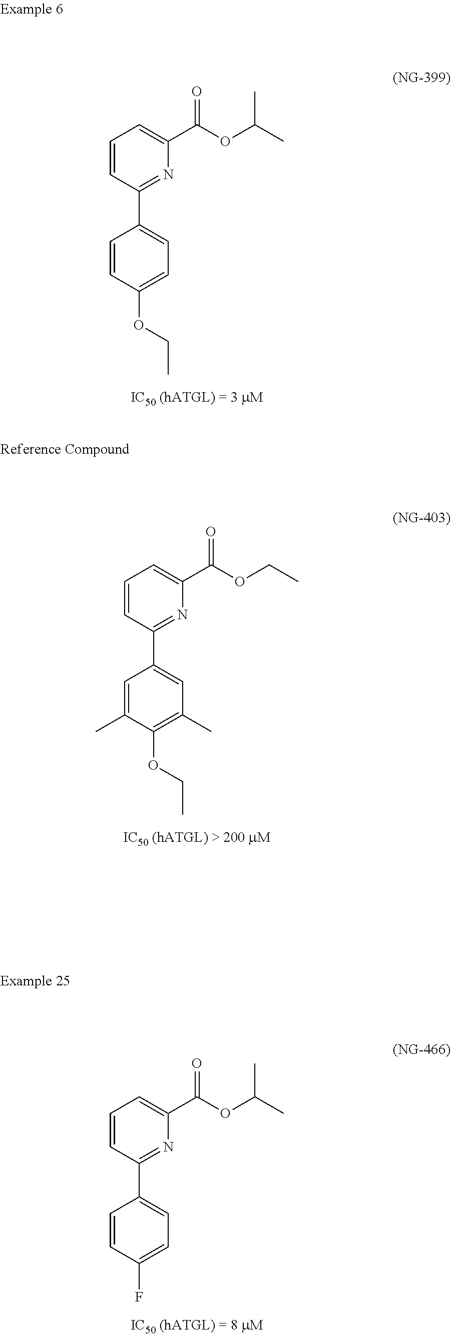

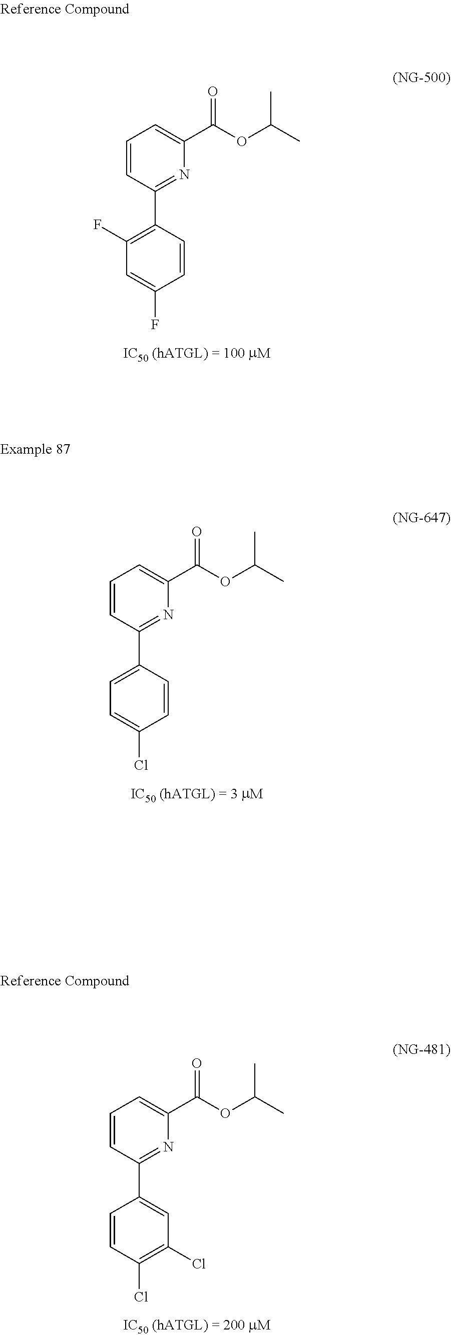 [SMILES: CC(C)OC(=O)c1cccc(-c2ccc(F)cc2F)n1 ; CC(C)OC(=O)c1cccc(-c2ccc(Cl)cc2)n1 ; CC(C)OC(=O)c1cccc(-c2ccc(Cl)c(Cl)c2)n1]

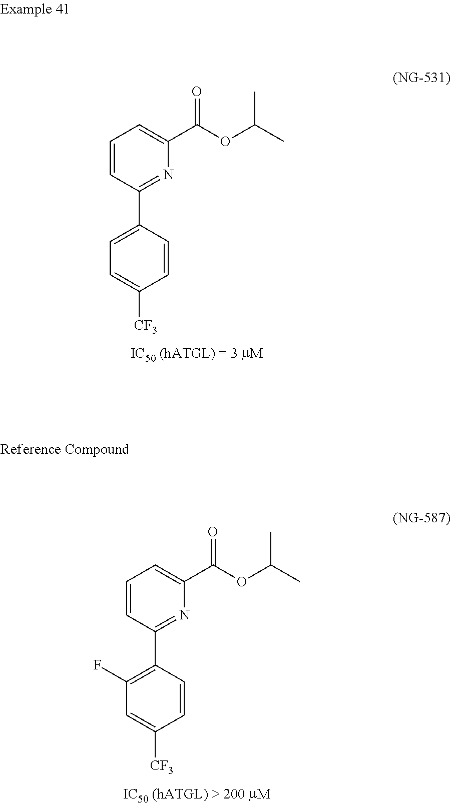 [SMILES: CC(C)OC(=O)c1cccc(-c2ccc(C(F)(F)F)cc2)n1 ; CC(C)OC(=O)c1cccc(-c2ccc(C(F)(F)F)cc2F)n1]

Moreover, it has also been found that the compounds of formula (I) containing a pyridine ring have a particularly advantageous inhibitory activity on human ATGL if the pyridine ring carries hydrogen atoms in positions 3 and 5, as reflected by the following examples:

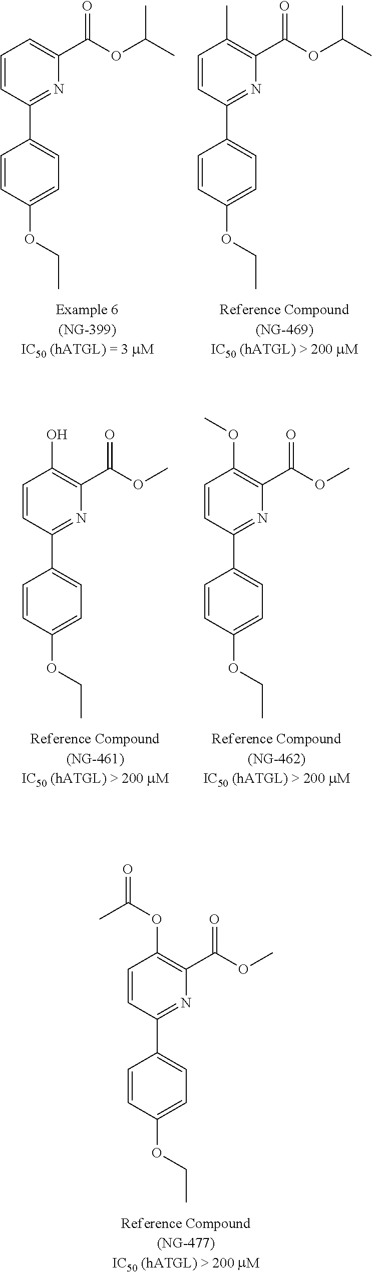

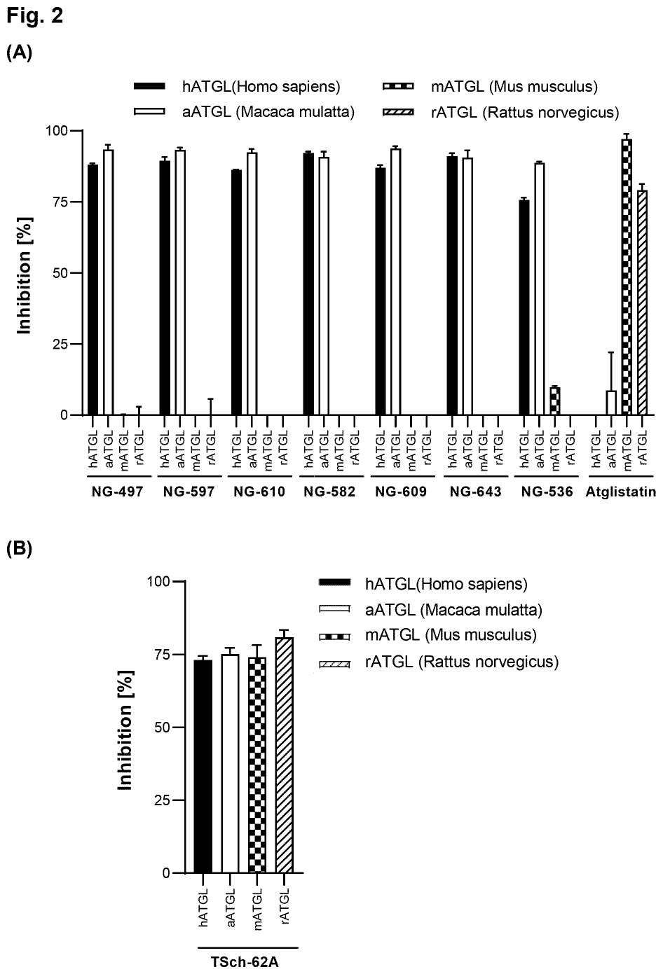

As demonstrated in Example 237 and FIG. 2, the present invention also provides compounds that inhibit both human ATGL and murine ATGL. Such cross-species activity is particularly advantageous for the preclinical development of the corresponding compounds, as their pharmacological and toxicological properties can be readily assessed in mouse models.

As explained above, the present invention provides a compound of formula (I) or a pharmaceutically acceptable salt or solvate thereof:

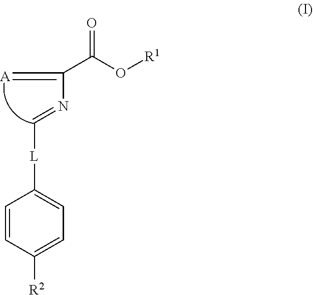

In formula (I), the group A is —CH═C(R^{A1})—CH═ or —S—C(R^{A2})═.

L is selected from a covalent bond, C₁₋₅ alkylene, C₂₋₅ alkenylene, and C₂₋₅ alkynylene, wherein one —CH₂— unit comprised in said C₁₋₅ alkylene, said C₂₋₅ alkenylene or said C₂₋₅ alkynylene is optionally replaced by —O—.

R¹ is selected from C₁₋₁₀ alkyl, C₂₋₁₀ alkenyl, C₂₋₁₀ alkynyl, carbocyclyl, and heterocyclyl, wherein said alkyl, said alkenyl and said alkynyl are each optionally substituted with one or more groups R^{Alk}, and wherein said carbocyclyl and said heterocyclyl are each optionally substituted with one or more groups R^{Cyc}.

R² is selected from hydrogen, C₁₋₁₀ alkyl, C₂₋₁₀ alkenyl, C₂₋₁₀ alkynyl, —(C₀₋₄ alkylene)-OH, —(C₀₋₄ alkylene)-O(C₁₋₁₀ alkyl), —(C₀₋₄ alkylene)-O(C₁₋₁₀ alkylene)-OH, —(C₀₋₄ alkylene)-O(C₁₋₁₀ alkylene)-O(C₁₋₅ alkyl), —(C₀₋₄ alkylene)-O(C₁₋₅ alkylene)-O(C₁₋₅ alkylene)-OH, —(C₀₋₄ alkylene)-O(C₁₋₅ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(C₁₋₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—O—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}-R^{X}, wherein said C₁₋₁₀ alkyl, said C₂₋₁₀ alkenyl, said C₂₋₁₀ alkynyl, each alkyl moiety in any of the aforementioned groups, and each alkylene moiety in any of the aforementioned groups are each optionally substituted with one or more groups R^{Alk}, and wherein the carbocyclyl moiety in said -L^{X}-carbocyclyl and the heterocyclyl moiety in said -L^{X}-heterocyclyl are each optionally substituted with one or more groups R^{Cyc}.

R^{A1} and R^{A2} are each independently selected from hydrogen, C₁₋₅ alkyl, C₂₋₅ alkenyl, C₂₋₅ alkynyl, —(C₀₋₄ alkylene)-OH, —(C₀₋₄ alkylene)-O(C₁₋₅ alkyl), —(C₀₋₄ alkylene)-O(C₁₋₅ alkylene)-OH, —(C₀₋₄ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(C₁₋₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—CO—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups R^{Cyc}.

Each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups R^{Cyc}.

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl).

Each L^{X} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{X} is independently selected from hydrogen, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅alkyl), —SO—(C₁₋₅ alkyl), carbocyclyl, and heterocyclyl, wherein said carbocyclyl and said heterocyclyl are each optionally substituted with one or more groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl).

The present invention also relates to a pharmaceutical composition comprising a compound of formula (I) or a pharmaceutically acceptable salt or solvate thereof, in combination with a pharmaceutically acceptable excipient. Accordingly, the invention relates to a compound of formula (I) or a pharmaceutically acceptable salt or solvate thereof, or a pharmaceutical composition comprising any of the aforementioned entities and a pharmaceutically acceptable excipient, for use as a medicament.

The invention further relates to a compound of formula (I) or a pharmaceutically acceptable salt or solvate thereof, or a pharmaceutical composition comprising any of the aforementioned entities and a pharmaceutically acceptable excipient, for use in the treatment or prevention of a disease/disorder, particularly a disease/disorder mediated by ATGL or a disease/disorder in which ATGL is implicated. The suitability of ATGL inhibitors for the treatment or prevention of such diseases/disorders has been discussed in the literature, including, e.g., in: WO 2014/114649; Mayer N et al., Nat Chem Biol, 2013, 9(12):785-787; Schweiger M et al., Nat Commun, 2017, 8:14859; Schreiber R et al., Proc Natl Acad Sci USA, 2015, 112(45):13850-13855; Zhou H et al., JCI Insight, 2019, 5. pii: 129781; Kozusko K et al, Diabetes, 2015, 64(1):299-310; Parajuli N et al, Am J Physiol Heart Circ Physiol, 2018, 315(4):H879-H884; Salatzki J et al, PLoS Genet, 2018, 14(1):e1007171; and the further references cited in each of the aforementioned documents.

The disease/disorder to be treated or prevented in accordance with the present invention is preferably a lipid metabolism disorder, including, e.g., obesity, non-alcoholic fatty liver disease (NAFLD; including non-alcoholic fatty liver (NAFL) or non-alcoholic steatohepatitis (NASH)), type 2 diabetes, insulin resistance, glucose intolerance, hypertriglyceridemia, metabolic syndrome (combined obesity, high blood pressure, glucose intolerance, and hypertriglyceridemia), cardiac and skeletal muscle steatosis (Mayer N et al., Nat Chem Biol, 2013, 9(12):785-787; Schweiger M et al, Nat Commun, 2017, 8:14859; Schreiber R et al., Proc Natl Acad Sci USA, 2015, 112(45):13850-13855), congenital generalized lipodystrophy (such as Beradinelli-Seip syndrome; Zhou H et al., JCI Insight, 2019, 5. pii: 129781), familial partial lipodystrophy (such as PLIN1 mutations; Kozusko K et al, Diabetes, 2015, 64(1):299-310), acquired lipodystrophy syndrome (generalized or partial), atherosclerosis, or heart failure (Parajuli N et al, Am J Physiol Heart Circ Physiol, 2018, 315(4):H879-H884; Salatzki J et al., PLoS Genet, 2018, 14(1):e1007171). The present invention thus relates to a compound of formula (I) or a pharmaceutically acceptable salt or solvate thereof, or a pharmaceutical composition comprising any of the aforementioned entities and a pharmaceutically acceptable excipient, for use in the treatment or prevention of any one of the aforementioned diseases/disorders (preferably in a human subject/patient).

Moreover, the present invention relates to the use of a compound of formula (I) or a pharmaceutically acceptable salt or solvate thereof in the preparation of a medicament for the treatment or prevention of a disease/disorder (particularly an ATGL-mediated disease/disorder), wherein said disease/disorder is preferably a lipid metabolism disorder, including, e.g., obesity, non-alcoholic fatty liver disease (NAFLD; including non-alcoholic fatty liver (NAFL) or non-alcoholic steatohepatitis (NASH)), type 2 diabetes, insulin resistance, glucose intolerance, hypertriglyceridemia, metabolic syndrome (combined obesity, high blood pressure, glucose intolerance, and hypertriglyceridemia), cardiac and skeletal muscle steatosis, congenital generalized lipodystrophy (such as Beradinelli-Seip syndrome), familial partial lipodystrophy (such as PLIN1 mutations), acquired lipodystrophy syndrome (generalized or partial), atherosclerosis, or heart failure.

The invention likewise relates to a method of treating or preventing a disease/disorder (particularly an ATGL-mediated disease/disorder), the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt or solvate thereof, or of the pharmaceutical composition, is to be administered in accordance with this method. The disease/disorder to be treated or prevented is preferably a lipid metabolism disorder, including, e.g., obesity, non-alcoholic fatty liver disease (NAFLD; including non-alcoholic fatty liver (NAFL) or non-alcoholic steatohepatitis (NASH)), type 2 diabetes, insulin resistance, glucose intolerance, hypertriglyceridemia, metabolic syndrome (combined obesity, high blood pressure, glucose intolerance, and hypertriglyceridemia), cardiac and skeletal muscle steatosis, congenital generalized lipodystrophy (such as Beradinelli-Seip syndrome), familial partial lipodystrophy (such as PLIN1 mutations), acquired lipodystrophy syndrome (generalized or partial), atherosclerosis, or heart failure.

The present invention furthermore relates to the use of a compound of formula (I) or a pharmaceutically acceptable salt or solvate thereof as an ATGL inhibitor in research, i.e., as a research tool compound for inhibiting ATGL, particularly human ATGL. Accordingly, the invention refers to the in vitro use of a compound of formula (I) or a pharmaceutically acceptable salt or solvate thereof as an ATGL inhibitor and, in particular, to the in vitro use of a compound of formula (I) or a pharmaceutically acceptable salt or solvate thereof as an inhibitor of human ATGL. The invention likewise relates to the in vitro use of a compound of formula (I) or a pharmaceutically acceptable salt or solvate thereof as a research tool compound acting as an ATGL inhibitor, particularly as an inhibitor of human ATGL. The invention further relates to a method, particularly an in vitro method, of inhibiting ATGL (particularly human ATGL), the method comprising the application of a compound of formula (I) or a pharmaceutically acceptable salt or solvate thereof. The invention also relates to a method of inhibiting ATGL (particularly human ATGL), the method comprising applying a compound of formula (I) or a pharmaceutically acceptable salt or solvate thereof to a test sample (e.g., a biological sample) or a test animal (i.e., a non-human test animal). The invention further refers to a method, particularly an in vitro method, of inhibiting ATGL (particularly human ATGL) in a sample (e.g., a biological sample), the method comprising applying a compound of formula (I) or a pharmaceutically acceptable salt or solvate thereof to said sample. The present invention likewise provides a method of inhibiting ATGL (particularly human ATGL), the method comprising contacting a test sample (e.g., a biological sample) or a test animal (i.e., a non-human test animal) with a compound of formula (I) or a pharmaceutically acceptable salt or solvate thereof. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compounds of formula (I) according to the present invention, as well as pharmaceutically acceptable salts and solvates thereof, will be described in more detail in the following:

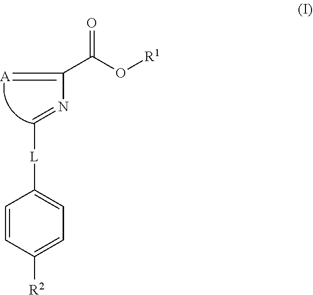

In formula (I), the group -A= is —CH═C(R^{A1})—CH═ or —S—C(R^{A2})═.

If A is —CH═C(R^{A1})—CH═, then the compound of formula (I) has the following structure:

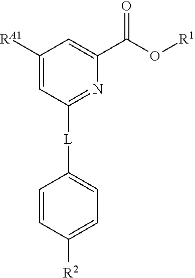

Conversely, if A is —S—C(R^{A2})═, then the compound of formula (I) has the following structure:

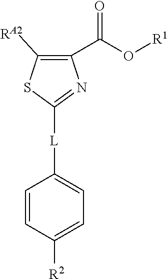

In formula (I), L is selected from a covalent bond, C₁₋₅ alkylene, C₂₋₅ alkenylene, and C₂₋₅ alkynylene, wherein one —CH₂— unit comprised in said C₁₋₅ alkylene, said C₂₋₅ alkenylene or said C₂₋₅ alkynylene is optionally replaced by —O—.

Preferably, L is selected from a covalent bond, C₁₋₅ alkylene (e.g., —CH₂CH₂—), C₂₋₅ alkenylene (e.g., —CH═CH—), and C₂₋₅ alkynylene. More preferably, L is a covalent bond.

R¹ is selected from C₁₋₁₀ alkyl, C₂₋₁₀ alkenyl, C₂₋₁₀ alkynyl, carbocyclyl, and heterocyclyl, wherein said alkyl, said alkenyl and said alkynyl are each optionally substituted with one or more (e.g., one, two or three) groups R^{Alk}, and wherein said carbocyclyl and said heterocyclyl are each optionally substituted with one or more (e.g., one, two or three) groups R^{Cyc}.

Preferably, R¹ is selected from C₁₋₆ alkyl, C₁₋₆ haloalkyl (e.g., —CH₂CF₃ or —CH(—CH₂F)—CH₂F), C₂₋₆ alkenyl, C₂₋₆ alkynyl, cycloalkyl, heterocycloalkyl, —(C₀₋₃ alkylene)-phenyl (e.g., benzyl) and —(C₀₋₃ alkylene)-heteroaryl (e.g., —CH₂-furanyl, —CH₂-thiophenyl, or —CH₂-pyridinyl). More preferably, R¹ is selected from C₁₋₆ alkyl, C₂₋₆ alkenyl, C₂₋₆ alkynyl, cycloalkyl, and heterocycloalkyl. In particular, said alkyl, said alkenyl or said alkynyl may be branched, i.e., R¹ may be a branched C₃₋₆ alkyl (e.g., isopropyl, sec-butyl, or sec-pentyl), a branched C₃₋₆ alkenyl (e.g., —CH(CH₃)—CH═CH₂), or a branched C₃₋₆ alkynyl (e.g., —CH(CH₃)—C≡CH). Even more preferably, R¹ is selected from C₁₋₅ alkyl, C₂₋₅ alkenyl, C₂₋₅ alkynyl, C₃₋₆ cycloalkyl, and a 4- to 6-membered heterocycloalkyl. Yet even more preferably, R¹ is selected from methyl, ethyl, n-propyl, isopropyl, n-butyl, isobutyl, sec-butyl, tert-butyl, sec-pentyl, sec-isopentyl, tert-pentyl, —CH₂—CH═CH₂, —CH₂—C(CH₃)═CH₂, —CH₂—CH═CH₂CH₃, —CH₂—C≡CH, —CH(CH₃)—C≡CH, cyclopropyl, cyclobutyl, cyclopentyl, and tetrahydrofuranyl (e.g., tetrahydrofuran-3-yl). Still more preferably, R¹ is selected from ethyl, isopropyl, —CH₂—CH═CH₂, —CH₂—C(CH₃)═CH₂, —CH(CH₃)—C≡CH, and cyclopropyl. It is particularly preferred that R¹ is ethyl or isopropyl.

R² is selected from hydrogen, C₁₋₁₀ alkyl, C₂₋₁₀ alkenyl, C₂₋₁₀ alkynyl, —(C₀₋₄ alkylene)-OH, —(C₀₋₄ alkylene)-O(C₁₋₁₀ alkyl), —(C₀₋₄ alkylene)-O(C₁₋₁₀ alkylene)-OH, —(C₀₋₄ alkylene)-O(C₁₋₁₀ alkylene)-O(C₁₋₅ alkyl), —(C₀₋₄ alkylene)-O(C₁₋₅ alkylene)-O(C₁₋₅ alkylene)-OH, —(C₀₋₄ alkylene)-(C₁₋₅ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(C₁₋₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—N—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—CO—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}-R^{X}, wherein said C₁₋₁₀ alkyl, said C₂₋₁₀ alkenyl, said C₂₋₁₀ alkynyl, each alkyl moiety in any of the aforementioned groups, and each alkylene moiety in any of the aforementioned groups are each optionally substituted with one or more (e.g., one, two or three) groups R^{Alk}, and wherein the carbocyclyl moiety in said -L^{X}-carbocyclyl and the heterocyclyl moiety in said -L^{X}-heterocyclyl are each optionally substituted with one or more (e.g., one, two or three) groups R^{Cyc}.

Preferably, R² is selected from hydrogen, C₁₋₁₀ alkyl, —(C₀₋₄ alkylene)-O(C₁₋₁₀ alkyl), —(C₀₋₄ alkylene)-O(C₁₋₁₀ alkylene)-O(C₁₋₅ alkyl), —(C₀₋₄ alkylene)-O(C₁₋₅ alkylene)-O(C₁₋₅ alkylene)-O(C₁₋₅ alkyl), —O(C₂₋₄ alkenyl), —(C₀₋₄ alkylene)-S(C₁₋₅ alkyl), —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-SO₂—(C₁₋₅ alkyl), halogen, C₁₋₅ haloalkyl, —(C₀₋₄ alkylene)-O—(C₁₋₅ haloalkyl), -L^{X}-aryl, -L^{X}-cycloalkyl, -L^{X}-heteroaryl, and -L^{X}-heterocycloalkyl, wherein the aryl moiety in said -L^{X}-aryl, the cycloalkyl moiety in said -L^{X}-cycloalkyl, the heteroaryl moiety in said -L^{X}-heteroaryl, and the heterocycloalkyl moiety in said -L^{X}-heterocycloalkyl are each optionally substituted with one or more groups R^{Cyc}. More preferably, R² is selected from C₁₋₁₀ alkyl, —O(C₁₋₁₀ alkyl), —(C₀₋₄ alkylene)-O(C₁₋₁₀ alkyl), —O(C₁₋₁₀ alkylene)-O(C₁₋₅ alkyl), —(C₀₋₄ alkylene)-O(C₁₋₁₀ alkylene)-O(C₁₋₅ alkyl), —O(C₁₋₅ alkylene)-O(C₁₋₅ alkylene)-O(C₁₋₅ alkyl), —(C₀₋₄ alkylene)-O(C₁₋₅ alkylene)-O(C₁₋₅ alkylene)-O(C₁₋₅ alkyl), —O(C₂₋₄ alkenyl), —S(C₁₋₅ alkyl), —COO—(C₁₋₅ alkyl), —CO—N(C₁₋₅ alkyl)(C₁₋₅ alkyl), —CO—N(C₁₋₅ alkyl)-O—(C₁₋₅ alkyl), —SO₂—(C₁₋₅ alkyl), halogen, C₁₋₅ haloalkyl, —O—(C₁₋₅haloalkyl), -L^{X}-aryl, -L^{X}-cycloalkyl (e.g., cycloalkyl or —O-cycloalkyl), -L^{X}-heteroaryl (e.g., heteroaryl, such as pyridinyl), and -L^{X}-heterocycloalkyl (e.g., heterocycloalkyl or —O— heterocycloalkyl, such as —O-(tetrahydropyran-2-yl)), wherein the aryl moiety in said -L^{X}-aryl, the cycloalkyl moiety in said -L^{X}-cycloalkyl, the heteroaryl moiety in said -L^{X}-heteroaryl, and the heterocycloalkyl moiety in said -L^{X}-heterocycloalkyl are each optionally substituted with one or more groups R^{Cyc}. Even more preferably, R² is selected from —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —CH(—CH₃)CH₂CH₃, —O—CH₃, —O—CH₂CH₃, —O—(CH₂)₂CH₃, —O—(CH₂)₃CH₃, —O—(CH₂)₄CH₃, —O—(CH₂)₅CH₃, —O—(CH₂)₆CH₃, —O—(CH₂)₇CH₃, —O—CH(—CH₃)—CH₃, —O—CH(—CH₃)—CH₂CH₃, —O—CH₂CH(—CH₃)—CH₃, —CH₂—O—CH₃, —CH₂CH₂—O—CH₃, —CH(—CH₃)—O—CH₃, —CH₂CH₂—O—CH₂CH₃, —O—CH₂—O—CH₃, —O—CH₂CH₂—O—CH₃, —O—CH₂CH₂—O—CH₂CH₃, —O—CH₂CH₂—O—CH₂CH₂—O—CH₃, —O—CH₂CH₂—O—CH₂CH₂—O—CH₂CH₃, —O—CH₂CH═CH₂, —S—CH₃, —S—CH₂CH₃, —COO—CH₂CH₃, —CO—N(—CH₃)—CH₃, —CO—N(—CH₃)—O—CH₃, —SO₂—CH₂CH₃, halogen (e.g., —F or —Cl), —CF₃, —CH₂CF₃, —O—CF₃, —O—CH₂CF₃, —CH₂CH₂-phenyl, —CH═CH-phenyl, —C≡C-phenyl, pyridin-3-yl, and —O-(tetrahydropyran-2-yl), wherein said pyridin-3-yl, the phenyl moiety in said —CH₂CH₂-phenyl, in said —CH═CH-phenyl and in said —C≡C-phenyl, and the tetrahydropyranyl moiety in said —O-(tetrahydropyran-2-yl) are each optionally substituted with one or more groups R^{Cyc}. Yet even more preferably, R² is selected from —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —O—CH₂CH₃, —O—(CH₂)₂CH₃, —O—(CH₂)₃CH₃, —O—(CH₂)₄CH₃, —O—(CH₂)₅CH₃, —O—(CH₂)₆CH₃, —O—(CH₂)₇CH₃, —O—CH(—CH₃)—CH₃, —CH₂—O—CH₃, —CH₂CH₂—O—CH₃, —CH(—CH₃)—O—CH₃, —O—CH₂CH₂—O—CH₃, —O—CH₂CH₂—O—CH₂CH₃, —S—CH₃, —C, —CF₃, —O—CH₂CF₃, —CH₂CH₂-phenyl, and —C≡C-phenyl. Still more preferably, R² is selected from —CH₂CH₃, —(CH₂)₂CH₃, —(CH₂)₃CH₃, —(CH₂)₄CH₃, —(CH₂)₅CH₃, —(CH₂)₆CH₃, —O—CH₂CH₃, —O—(CH₂)₂CH₃, —O—(CH₂)₃CH₃, —O—(CH₂)₄CH₃, —O—(CH₂)₅CH₃, —O—(CH₂)₆CH₃, —CH₂—O—CH₃, and —CH(—CH₃)—O—CH₃. A particularly preferred example of R² is —O—CH₂CH₃.

R^{A1} and R^{A2} are each independently selected from hydrogen, C₁₋₅ alkyl, C₂₋₅ alkenyl, C₂₋₅ alkynyl, —(C₀₋₄ alkylene)-OH, —(C₀₋₄ alkylene)-O(C₁₋₅ alkyl), —(C₀₋₄ alkylene)-O(C₁₋₅ alkylene)-OH, —(C₀₋₄ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(CO₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—CO—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more (e.g., one, two or three) groups R^{Cyc}.

Preferably, R^{A1} is selected from hydrogen, —CH₃, —OCH₃, —CO—(C₁₋₅ alkyl) (e.g., —CO-methyl, —CO-ethyl, or —CO-isopropyl), halogen (e.g., —I), and piperidinyl (e.g., piperidin-1-yl). More preferably, R^{A1} is selected from hydrogen, —CH₃, —OCH₃, —CO—CH₃, and —I. Even more preferably, R^{A1} is selected from hydrogen, —CH₃, and —OCH₃. It is particularly preferred that R^{A1} is hydrogen.

Preferably, R^{A2} is selected from hydrogen, —CH₃, —OCH₃, —CO—(C₁₋₅ alkyl) (e.g., —CO-methyl, —CO-ethyl, or —CO-isopropyl), halogen (e.g., —I), and piperidinyl (e.g., piperidin-1-yl). More preferably, R^{A2} is selected from hydrogen, —CH₃, —OCH₃, —CO—CH₃, and —I. It is particularly preferred that R^{A2} is hydrogen.

Each R^{Alk} is independently selected from —OH, —O(C₁₋₅ alkyl), —(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more (e.g., one, two or three) groups R^{Cyc}.

Preferably, each R^{Alk} is independently selected from —OH, —O(C₁₋₅ alkyl), —O(C₁₋₅ alkylene)-OH, —(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-CO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl). More preferably, each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), and —CN.

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more (e.g., one, two or three) groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —(C₁₋₅ alkyl)(C₁₋₅ alkyl), halogen, C₁₋₅ haloalkyl, —O(C₁₋₅ haloalkyl), —CN, —CH, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl).

Preferably, each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl). More preferably, each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), and —CN.

Each L^{X} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more (e.g., one, two or three) groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more (e.g., one, two or three) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Preferably, each L^{X} is independently selected from a covalent bond, C₁₋₅ alkylene, C₂₋₅ alkenylene, and C₂₋₅ alkynylene, wherein one or more (e.g., one or two) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—. More preferably, each L^{X} is independently selected from a covalent bond and C₁₋₅ alkylene, wherein one or two —CH₂— units comprised in said alkylene are each optionally replaced by a group independently selected from —O—, —NH—, —N(C₁₋₅ alkyl)-, —CO—, —S—, —SO—, and —SO₂—.

Each R^{X} is independently selected from hydrogen, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), carbocyclyl, and heterocyclyl, wherein said carbocyclyl and said heterocyclyl are each optionally substituted with one or more (e.g., one, two or three) groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(CO₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —S₂—NH₂, —SO₂—NH(C₁₋₅ alkyl), —SO₂—N(C₁₋₅ alkyl)(C₁₋₅ alkyl), —NH—S₂—(C₁₋₅ alkyl), —N(C₁₋₅ alkyl)-SO₂—(C₁₋₅ alkyl), —S₂—(C₁₋₅ alkyl), and —SO—(C₁₋₅ alkyl).

Preferably, each R^{X} is independently selected from hydrogen, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —S₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl). More preferably, each R^{X} is independently selected from hydrogen, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), and —CN.

In a preferred aspect of the present invention, the following compounds are excluded:
ethyl 6-(4-methoxyphenyl)-2-pyridinecarboxylate;
ethyl 6-(4-hydroxyphenyl)-2-pyridinecarboxylate;
ethyl 6-(4-{[(3-fluorophenyl)methyl]oxy}phenyl)-2-pyridinecarboxylate;
methyl 2-phenylthiazole-4-carboxylate;
methyl 2-(4-ethoxyphenyl)thiazole-4-carboxylate;
dimethyl 2,2′-[oxybis(4,1-phenylene)]bis(thiazole-4-carboxylate);
dimethyl 2,2′-(1,4-phenylene)dithiazole-4-carboxylate;
ethyl 2-phenyl-5-chloro-thiazole-4-carboxylate;
ethyl 2-(4-methoxyphenyl)-5-chloro-thiazole-4-carboxylate;
ethyl 2-(phenylethynyl)-5-chloro-thiazole-4-carboxylate;
ethyl 2-phenyl-5-phenyl-thiazole-4-carboxylate;
ethyl 2-phenyl-5-vinyl-thiazole-4-carboxylate;
ethyl 2-phenyl-5-(2-pyridyl)-thiazole-4-carboxylate;
ethyl 2-phenyl-5-(phenylethynyl)-thiazole-4-carboxylate;
ethyl 2-(4-methoxyphenyl)-5-phenyl-thiazole-4-carboxylate;
2-phenyl-4-carbethoxythiazole;
2-(4′-methoxyphenyl)-4-carbethoxythiazole;
2-(4′-methylphenyl)-4-carbethoxythiazole;
2-(4′-carbomethoxyphenyl)-4-carbethoxythiazole;
2-(4′-chlorophenyl)-4-carbethoxythiazole;
2-benzyl-4-carbethoxythiazole; and
2-(2′-phenylethyl)-4-carbethoxythiazole.

It is particularly preferred that the above-mentioned compounds are excluded from formula (I). Accordingly, it is particularly preferred that the compound of formula (I) is not any one of the above-mentioned compounds or a pharmaceutically acceptable salt or solvate thereof.

It is furthermore preferred that the compound methyl 2-(4-cyanophenyl)thiazole-4-carboxylate is excluded. In particular, it is preferred that this compound is excluded from formula (I).

It is particularly preferred that the compound of formula (I) is one of the specific compounds described in the examples section of this specification, including any one of the compounds of Examples 1 to 236 described further below, either in non-salt form or as a pharmaceutically acceptable salt or solvate of the respective compound.

Accordingly, it is preferred that the compound of formula (I) is any one of the following compounds or a pharmaceutically acceptable salt or solvate thereof:

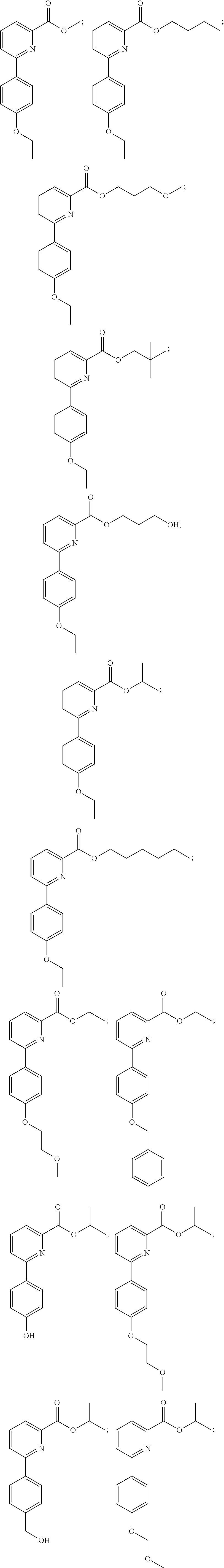

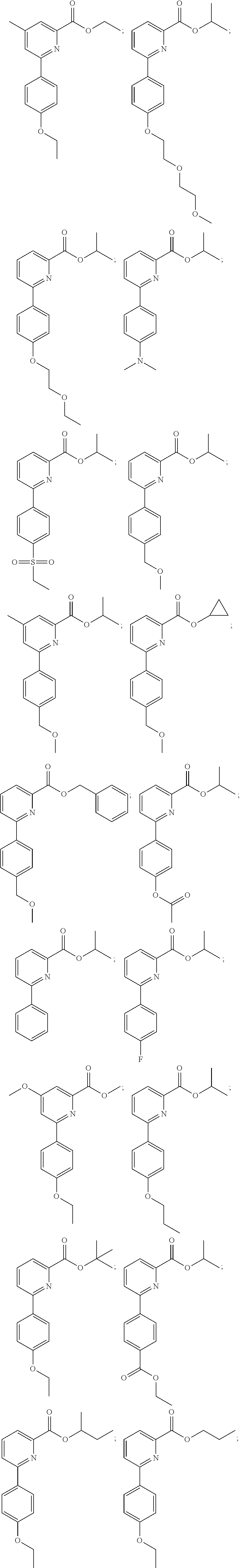

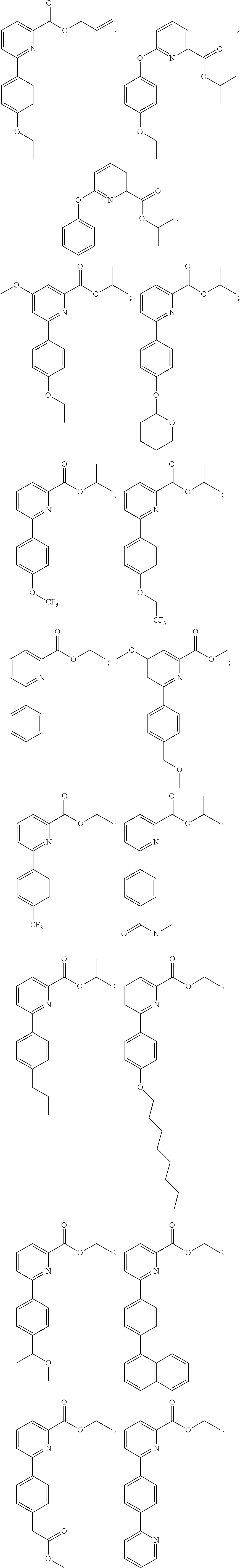

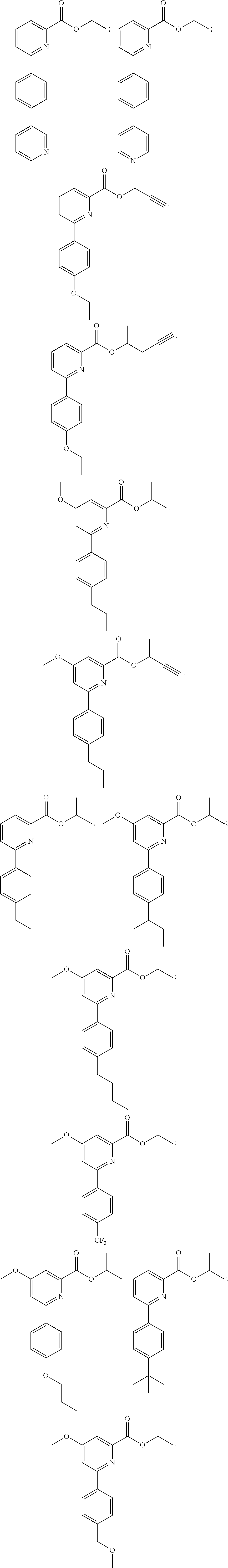

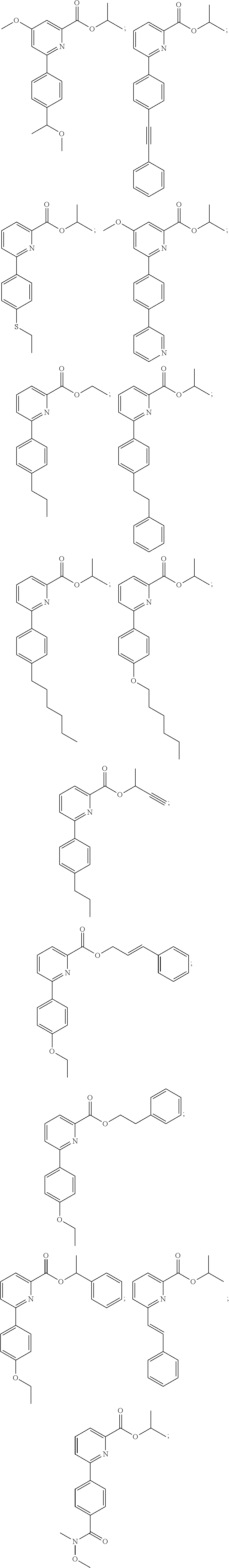

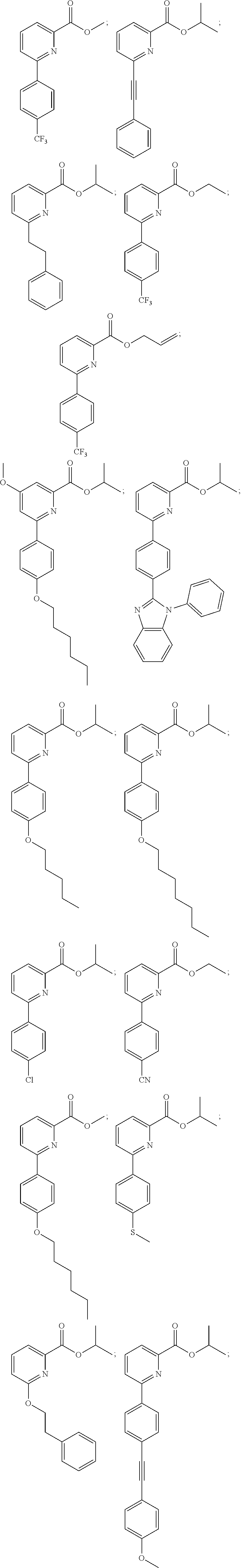

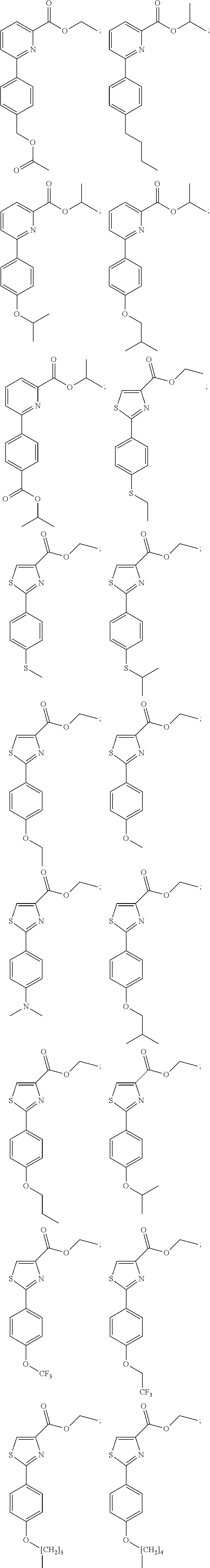

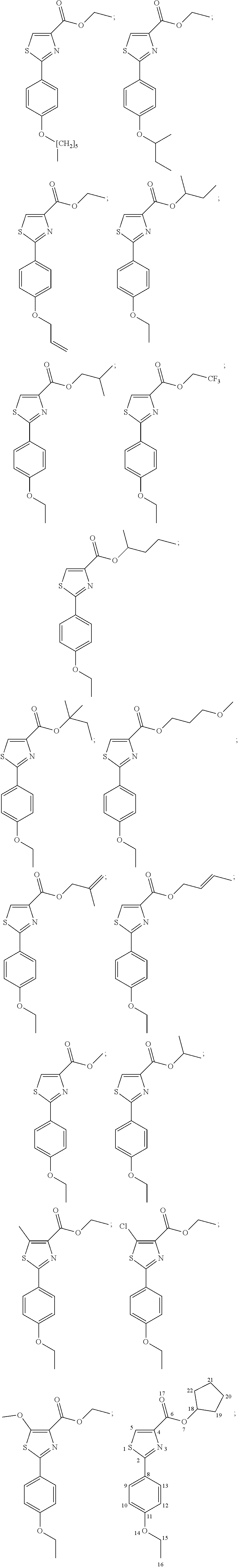

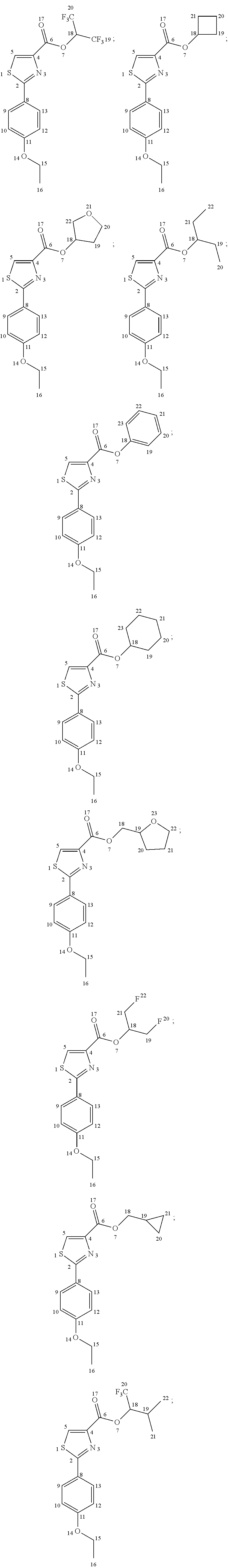

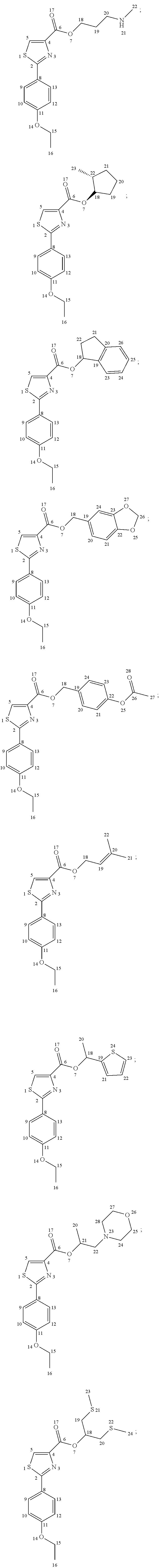

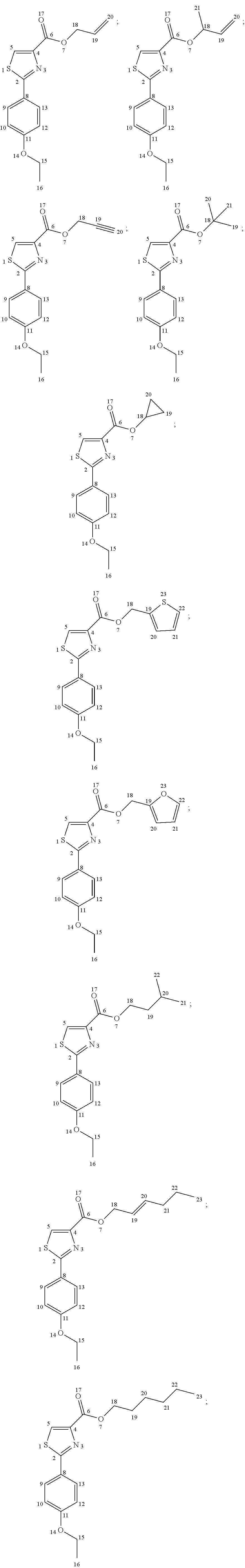

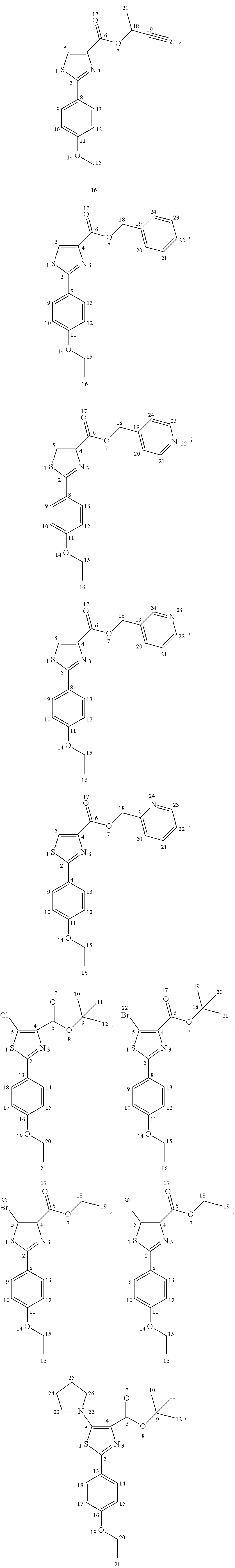

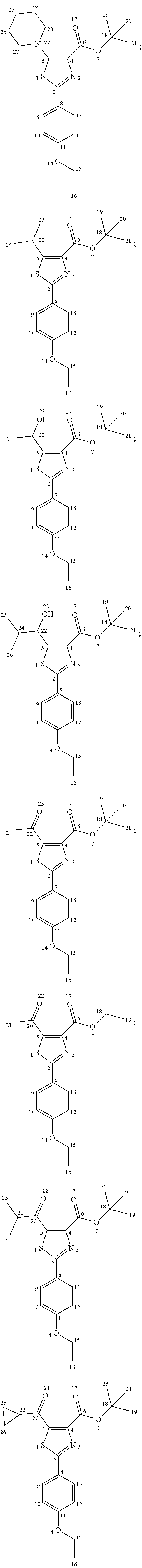

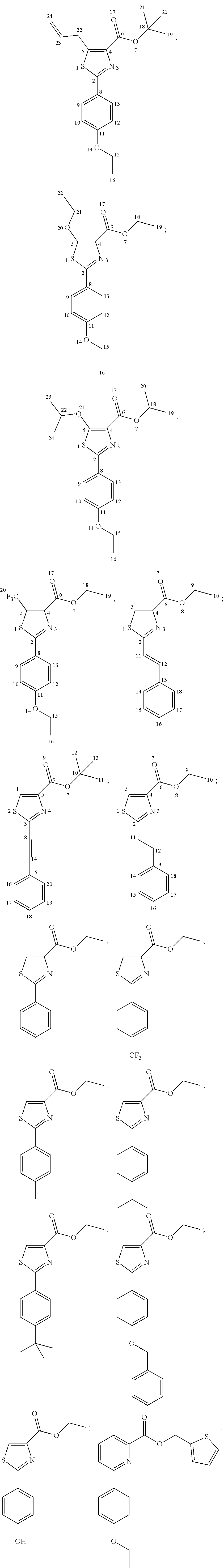

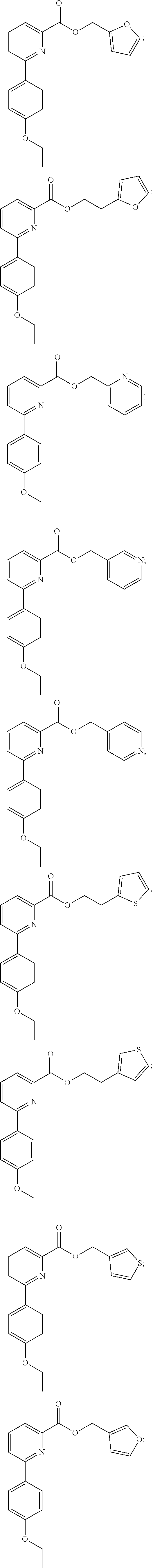

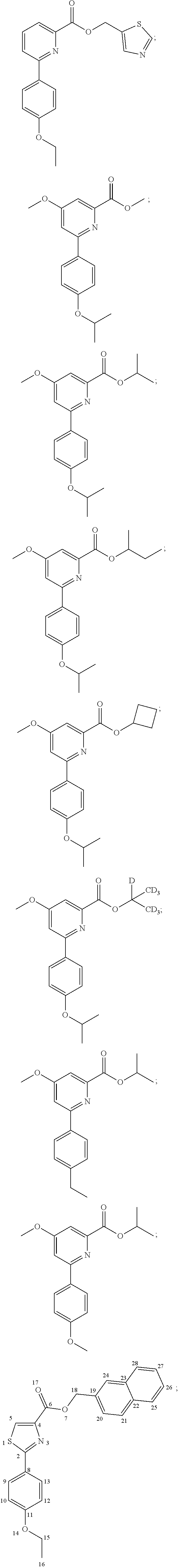

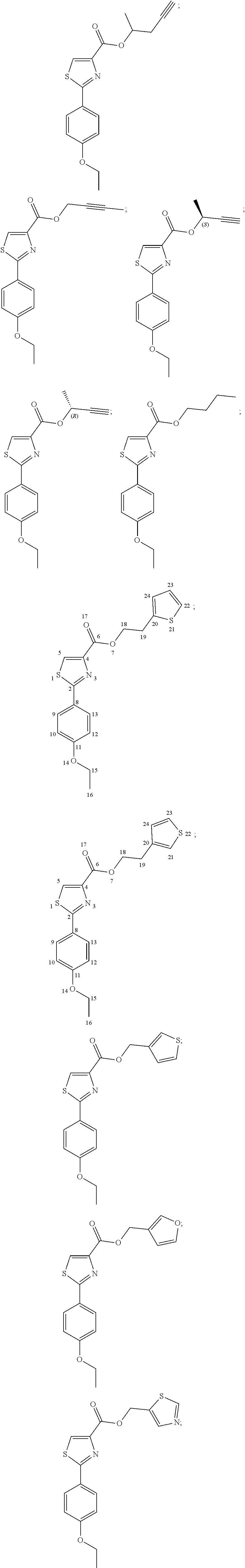

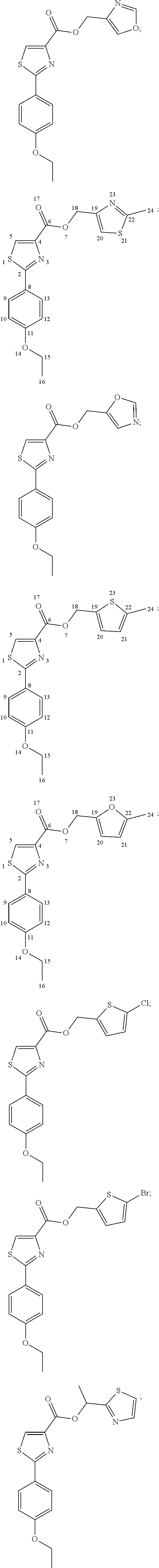

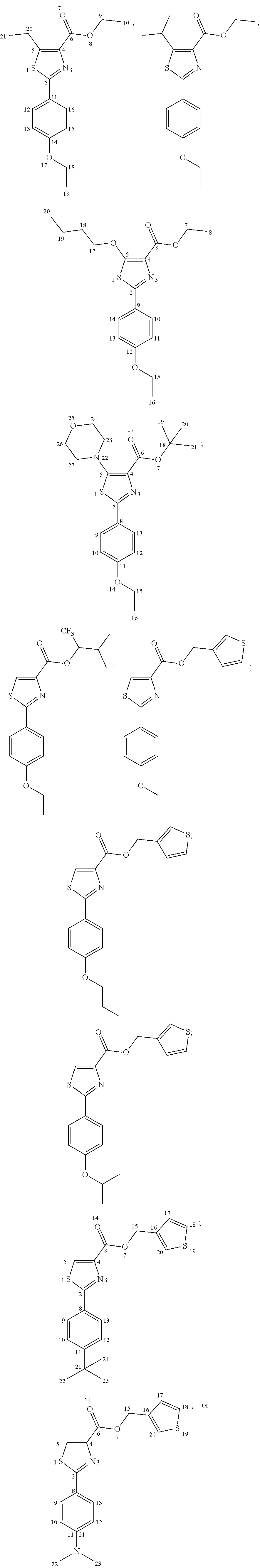

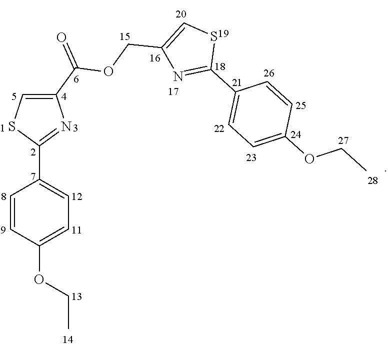 [SMILES: CCOc1ccc(-c2nc(COC(=O)c3csc(-c4ccc(OCC)cc4)n3)cs2)cc1]

Even more preferably, the compound of formula (I) is any one of the following compounds or a pharmaceutically acceptable salt or solvate thereof:

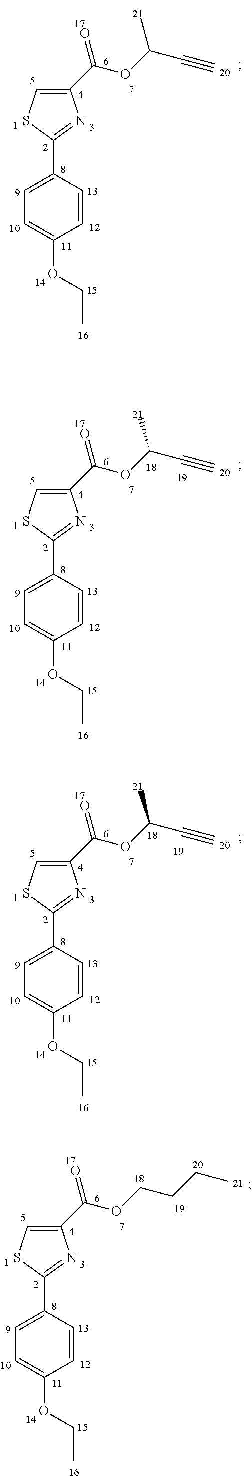 [SMILES: C#CC(C)OC(=O)c1csc(-c2ccc(OCC)cc2)n1 ; C#C[C@@H](C)OC(=O)c1csc(-c2ccc(OCC)cc2)n1 ; C#C[C@H](C)OC(=O)c1csc(-c2ccc(OCC)cc2)n1 ; CCCCOC(=O)c1csc(-c2ccc(OCC)cc2)n1]

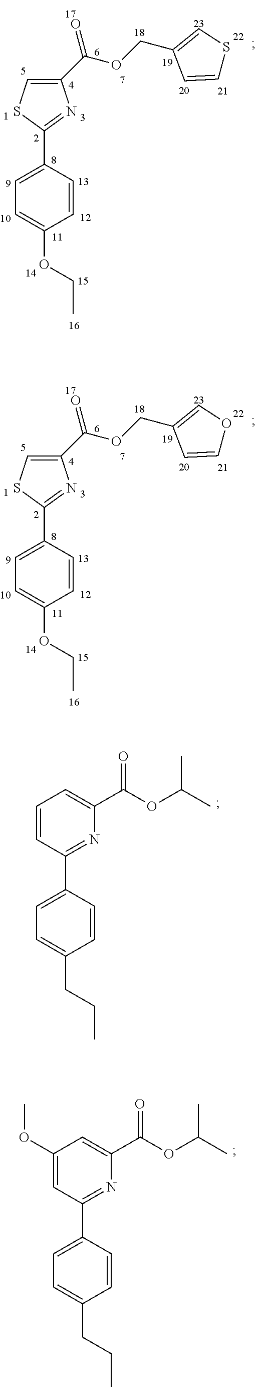 [SMILES: CCOc1ccc(-c2nc(C(=O)OCc3ccsc3)cs2)cc1 ; CCOc1ccc(-c2nc(C(=O)OCc3ccoc3)cs2)cc1 ; CCCc1ccc(-c2cccc(C(=O)OC(C)C)n2)cc1 ; CCCc1ccc(-c2cc(OC)cc(C(=O)OC(C)C)n2)cc1]

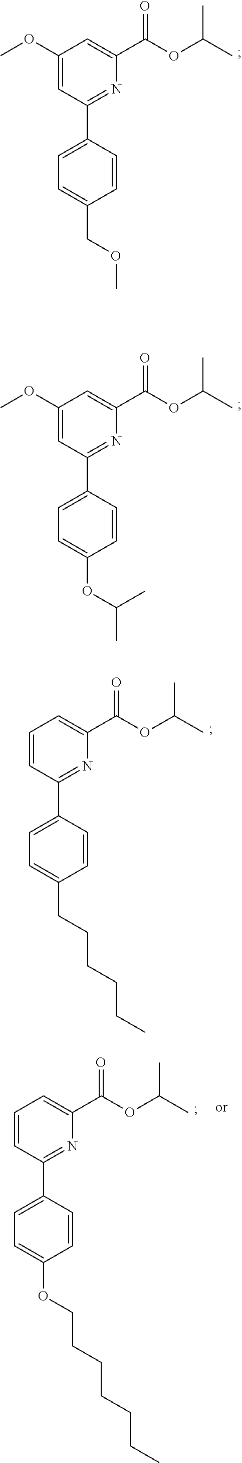

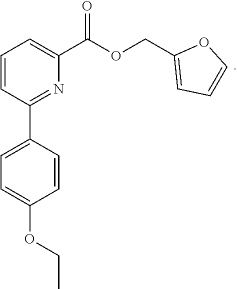 [SMILES: CCOc1ccc(-c2cccc(C(=O)OCc3ccco3)n2)cc1]

A particularly preferred example of the compound of formula (I) is the following compound:

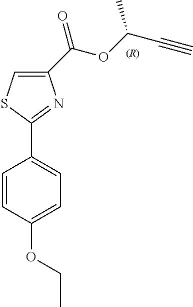

or a pharmaceutically acceptable salt or solvate thereof.

The present invention also relates to each one of the intermediates described in the examples section of this specification, including any one of these intermediates in non-salt form or in the form of a salt or solvate (e.g., a pharmaceutically acceptable salt or solvate) of the respective compound. Such intermediates can be used, in particular, in the synthesis of the compounds of formula (I).

As explained above, the present invention provides novel compounds, which are effective as inhibitors of ATGL and can thus be used, e.g., in the treatment or prevention of a lipid metabolism disorder, obesity, non-alcoholic fatty liver disease, type 2 diabetes, insulin resistance, glucose intolerance, hypertriglyceridemia, metabolic syndrome, cardiac and skeletal muscle steatosis, congenital generalized lipodystrophy, familial partial lipodystrophy, acquired lipodystrophy syndrome, atherosclerosis, or heart failure.

In particular, the present invention provides a compound of formula (I), as described and defined herein, or a pharmaceutically acceptable salt or solvate thereof, wherein -A= is —CH═C(R^{A1})—CH═ and the compound thus has the following formula:

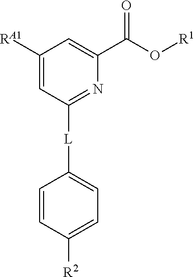

and further wherein the following compounds are excluded:
ethyl 6-(4-methoxyphenyl)-2-pyridinecarboxylate;
ethyl 6-(4-hydroxyphenyl)-2-pyridinecarboxylate; and
ethyl 6-(4-{[(3-fluorophenyl)methyl]oxy}phenyl)-2-pyridinecarboxylate.

The present invention further provides a compound of formula (I), as described and defined herein, or a pharmaceutically acceptable salt or solvate thereof, wherein -A= is —S—C(R^{A2})═ and the compound thus has the following formula:

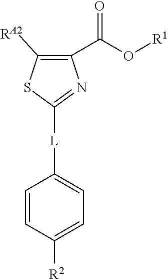

wherein R² is selected from hydrogen, C₁₋₁₀ alkyl, —(C₀₋₄ alkylene)-O(C₁₋₁₀ alkyl), —(C₀₋₄ alkylene)-O(C₁₋₁₀ alkylene)-O(C₁₋₅ alkyl), —(C₀₋₄ alkylene)-O(C₁₋₅ alkylene)-O(C₁₋₅ alkylene)-O(C₁₋₅ alkyl), —O(C₂₋₄ alkenyl), —(C₀₋₄ alkylene)-S(C₁₋₅ alkyl), —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-SO₂—(C₁₋₅ alkyl), halogen, C₁₋₅ haloalkyl, —(C₀₋₄ alkylene)-O—(C₁₋₅ fluoroalkyl), -L^{X}-aryl, -L^{X}-heteroaryl, cycloalkyl, and heterocycloalkyl, wherein the aryl moiety in said -L^{X}-aryl, the heteroaryl moiety in said -L^{X}-heteroaryl, said cycloalkyl, and said heterocycloalkyl are each optionally substituted with one or more groups R^{Cyc};

and further wherein the following compounds are excluded:
methyl 2-phenylthiazole-4-carboxylate;
methyl 2-(4-ethoxyphenyl)thiazole-4-carboxylate;
dimethyl 2,2′-[oxybis(4,1-phenylene)]bis(thiazole-4-carboxylate);
dimethyl 2,2′-(1,4-phenylene)dithiazole-4-carboxylate;
ethyl 2-phenyl-5-chloro-thiazole-4-carboxylate;
ethyl 2-(4-methoxyphenyl)-5-chloro-thiazole-4-carboxylate;
ethyl 2-(phenylethynyl)-5-chloro-thiazole-4-carboxylate;
ethyl 2-phenyl-5-phenyl-thiazole-4-carboxylate;
ethyl 2-phenyl-5-vinyl-thiazole-4-carboxylate;
ethyl 2-phenyl-5-(2-pyridyl)-thiazole-4-carboxylate;
ethyl 2-phenyl-5-(phenylethynyl)-thiazole-4-carboxylate;
ethyl 2-(4-methoxyphenyl)-5-phenyl-thiazole-4-carboxylate;
2-phenyl-4-carbethoxythiazole;
2-(4′-methoxyphenyl)-4-carbethoxythiazole;
2-(4′-methylphenyl)-4-carbethoxythiazole;
(4′-carbomethoxyphenyl)-4-carbethoxythiazole;
2-(4′-chlorophenyl)-4-carbethoxythiazole;
2-benzyl-4-carbethoxythiazole; and
2-(2′-phenylethyl)-4-carbethoxythiazole.

In this compound, it is preferred that R² is selected from C₁₋₁₀ alkyl, —O(C₁₋₁₀ alkyl), —(C₁₋₄ alkylene)-O(C₁₋₁₀ alkyl), —O(C₁₋₁₀ alkylene)-O(C₁₋₅ alkyl), —(C₁₋₄ alkylene)-O(C₁₋₁₀ alkylene)-O(C₁₋₅ alkyl), —O(C₁₋₅ alkylene)-O(C₁₋₅ alkylene)-O(C₁₋₅ alkyl), —(C₁₋₄ alkylene)-O(C₁₋₅ alkylene)-O(C₁₋₅ alkylene)-O(C₁₋₅ alkyl), —O(C₂₋₄ alkenyl), —S(C₁₋₅ alkyl), —COO—(C₁₋₅ alkyl), —CO—N(C₁₋₅ alkyl)(C₁₋₅ alkyl), —CO—N(C₁₋₅ alkyl)-O—(C₁₋₅ alkyl), —SO₂—(C₁₋₅ alkyl), halogen, C₁₋₅ haloalkyl, —O—(C₁₋₅ fluoroalkyl), -L^{X}-aryl, -L^{X}-heteroaryl, cycloalkyl, and heterocycloalkyl, wherein the aryl moiety in said -L^{X}-aryl, the heteroaryl moiety in said -L^{X}-heteroaryl, said cycloalkyl and said heterocycloalkyl are each optionally substituted with one or more groups R^{Cyc}; more preferably, R² is selected from —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —CH(—CH₃)CH₂CH₃, —O—CH₃, —O—CH₂CH₃, —O—(CH₂)₂CH₃, —O—(CH₂)₃CH₃, —(CH₂)₄CH₃, —O—(CH₂)₅CH₃, —O—(CH₂)₆CH₃, —O—(CH₂)₇CH₃, —O—CH(—CH₃)—CH₃, —O—CH(—CH₃)—CH₂CH₃, —O—CH₂CH(—CH₃)—CH₃, —CH₂—O—CH₃, —CH₂CH₂—O—CH₃, —CH(—CH₃)—O—CH₃, —CH₂CH₂—CH₂CH₃, —O—CH₂O—OCH₃, —O—CH₂CH₂—O—CH₃, —O—CH₂CH₂—O—CH₂CH₃, —O—CH₂CH₂—O—CH₂CH₂—O—CH₃, —O—CH₂CH₂—O—CH₂CH₂—O—CH₂CH₃, —O—CH₂CH═CH₂, —S—CH₃, —S—CH₂CH₃, —COO—CH₂CH₃, —CO—N(—CH₃)—CH₃, —CO—N(—CH₃)—O—CH₃, —SO₂—CH₂CH₃, halogen (e.g., —F or —Cl), —CF₃, —CH₂CF₃, —O—CF₃, —O—CH₂CF₃, —CH₂CH₂-phenyl, —CH═CH-phenyl, —C≡C-phenyl, and pyridin-3-yl, wherein said pyridin-3-yl and the phenyl moiety in said —CH₂CH₂-phenyl, in said —CH═CH— phenyl and in said —C≡C-phenyl are each optionally substituted with one or more groups R^{Cyc}; even more preferably, R² is selected from —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —O—CH₂CH₃, —O—(CH₂)₂CH₃, —O—(CH₂)₃CH₃, —O—(CH₂)₄CH₃, —O—(CH₂)₅CH₃, —O—(CH₂)₆CH₃, —O—(CH₂)₇CH₃, —O—CH(—CH₃)—CH₃, —CH₂—O—CH₃, —CH₂CH₂—O—CH₃, —CH(—CH₃)—O—CH₃, —O—CH₂CH₂—O—CH₃, —O—CH₂CH₂—O—CH₂CH₃, —S—CH₃, —Cl, —CF₃, —O—CH₂CF₃, —CH₂CH₂-phenyl, and —C≡C-phenyl; still more preferably, R² is selected from —CH₂CH₃, —(CH₂)₂CH₃, —(CH₂)₃CH₃, —(CH₂)₄CH₃, —(CH₂)₅CH₃, —(CH₂)₆CH₃, —O—CH₂CH₃, —O—(CH₂)₂CH₃, —O—(CH₂)₃CH₃, —O—(CH₂)₄CH₃, —O—(CH₂)₅CH₃, —O—(CH₂)₆CH₃, —CH₂—O—CH₃, and —CH(—CH₃)—O—CH₃; a particularly preferred example of R² is —O—CH₂CH₃.

In a first specific embodiment, the compound of formula (I) is a compound of the following formula (Ia) or a pharmaceutically acceptable salt or solvate thereof:

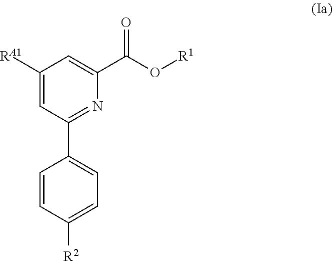

wherein the groups and variables in formula (Ia), including in particular R¹, R² and R^{A1}, have the same meanings, including the same preferred meanings, as described and defined herein above for the compound of formula (I).

In a second specific embodiment, the compound of formula (I) is a compound of formula (Ia), as depicted above, or a pharmaceutically acceptable salt or solvate thereof, wherein R⁴ is selected from ethyl, isopropyl, —CH₂—CH═CH₂, —CH₂—C(CH₃)═CH₂, —CH(CH₃)—C≡CH, and cyclopropyl, and further wherein R² and R^{A1} have the same meanings, including the same preferred meanings, as described and defined herein above for the compound of formula (I).

In a third specific embodiment, the compound of formula (I) is a compound of formula (Ia) or a pharmaceutically acceptable salt or solvate thereof, wherein R¹ is isopropyl, and further wherein R² and R^{A1} have the same meanings, including the same preferred meanings, as described and defined herein above for the compound of formula (I).

In a fourth specific embodiment, the compound of formula (I) is a compound of formula (Ia) or a pharmaceutically acceptable salt or solvate thereof, wherein R¹ is ethyl, and further wherein R² and R^{A1} have the same meanings, including the same preferred meanings, as described and defined herein above for the compound of formula (I).

In a fifth specific embodiment, the compound of formula (I) is a compound of formula (Ia) or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, wherein R^{A1} is selected from hydrogen, —CH₃, —OCH₃, —CO—CH₃, and —I (preferably R^{A1} is selected from hydrogen, —CH₃, and —OCH₃), and further wherein R² has the same meanings, including the same preferred meanings, as described and defined herein above for the compound of formula (I).

In a sixth specific embodiment, the compound of formula (I) is a compound of formula (Ia) or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, wherein R^{A1} is hydrogen, and further wherein R² has the same meanings, including the same preferred meanings, as described and defined herein above for the compound of formula (I).

In a seventh specific embodiment, the compound of formula (I) is a compound of formula (Ia) or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, wherein R^{A1} is —CH₃, and further wherein R² has the same meanings, including the same preferred meanings, as described and defined herein above for the compound of formula (I).

In an eighth specific embodiment, the compound of formula (I) is a compound of formula (Ia) or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, wherein R^{A1} is —OCH₃, and further wherein R² has the same meanings, including the same preferred meanings, as described and defined herein above for the compound of formula (I).

In a ninth specific embodiment, the compound of formula (I) is a compound of formula (Ia) or a pharmaceutically acceptable salt or solvate thereof, wherein R¹ is isopropyl, wherein R^{A1} is hydrogen, and wherein R² has the same meanings, including the same preferred meanings, as described and defined herein above for the compound of formula (I).

In a tenth specific embodiment, the compound of formula (I) is a compound of formula (Ia) or a pharmaceutically acceptable salt or solvate thereof, wherein R¹ is isopropyl, wherein R^{A1} is —CH₃, and wherein R² has the same meanings, including the same preferred meanings, as described and defined herein above for the compound of formula (I).

In an 11^{th} specific embodiment, the compound of formula (I) is a compound of formula (Ia) or a pharmaceutically acceptable salt or solvate thereof, wherein R¹ is isopropyl, wherein R^{A1} is —OCH₃, and wherein R² has the same meanings, including the same preferred meanings, as described and defined herein above for the compound of formula (I).

In a 12^{th} specific embodiment, the compound of formula (I) is a compound of formula (Ia) or a pharmaceutically acceptable salt or solvate thereof, wherein R¹ is ethyl, wherein R^{A1} is hydrogen, and wherein R² has the same meanings, including the same preferred meanings, as described and defined herein above for the compound of formula (I).

In a 13^{th} specific embodiment, the compound of formula (I) is a compound of formula (Ia) or a pharmaceutically acceptable salt or solvate thereof, wherein R¹ is ethyl, wherein R^{A1} is —CH₃, and wherein R² has the same meanings, including the same preferred meanings, as described and defined herein above for the compound of formula (I).

In a 14^{th} specific embodiment, the compound of formula (I) is a compound of formula (Ia) or a pharmaceutically acceptable salt or solvate thereof, wherein R¹ is ethyl, wherein R^{A1} is —OCH₃, and wherein R² has the same meanings, including the same preferred meanings, as described and defined herein above for the compound of formula (I).

In a 15^{th} specific embodiment, the compound of formula (I) is a compound of the following formula (Ib) or a pharmaceutically acceptable salt or solvate thereof:

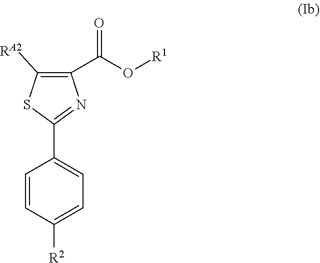

wherein the groups and variables in formula (Ib), including in particular R¹, R² and R^{A2}, have the same meanings, including the same preferred meanings, as described and defined herein above for the compound of formula (I).

In a 16^{th} specific embodiment, the compound of formula (I) is a compound of formula (Ib), as depicted above, or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, and further wherein R² and R^{A2} have the same meanings, including the same preferred meanings, as described and defined herein above for the compound of formula (I).

In a 17^{th} specific embodiment, the compound of formula (I) is a compound of formula (Ib) or a pharmaceutically acceptable salt or solvate thereof, wherein R¹ is isopropyl, and further wherein R² and R^{A2} have the same meanings, including the same preferred meanings, as described and defined herein above for the compound of formula (I).

In an 18^{th} specific embodiment, the compound of formula (I) is a compound of formula (Ib) or a pharmaceutically acceptable salt or solvate thereof, wherein R¹ is ethyl, and further wherein R² and R^{A2} have the same meanings, including the same preferred meanings, as described and defined herein above for the compound of formula (I).

In a 19^{th} specific embodiment, the compound of formula (I) is a compound of formula (Ib) or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, wherein R^{A2} is selected from hydrogen, —CH₃, —OCH₃, —CO—CH₃, and —I, and further wherein R² has the same meanings, including the same preferred meanings, as described and defined herein above for the compound of formula (I).

In a 20^{th} specific embodiment, the compound of formula (I) is a compound of formula (Ib) or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, wherein R^{A2} is hydrogen, and further wherein R² has the same meanings, including the same preferred meanings, as described and defined herein above for the compound of formula (I).

In a 21^{st} specific embodiment, the compound of formula (I) is a compound of formula (Ib) or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, wherein R^{A2} is —CH₃, and further wherein R² has the same meanings, including the same preferred meanings, as described and defined herein above for the compound of formula (I).

In a 22^{nd} specific embodiment, the compound of formula (I) is a compound of formula (Ib) or a pharmaceutically acceptable salt or solvate thereof, wherein R¹ is selected from ethyl, isopropyl, —CH₂—CH═CH₂, —CH₂—C(CH₃)═CH₂, —CH(CH₃)—C≡CH, and cyclopropyl, wherein R^{A2} is —OCH₃, and further wherein R² has the same meanings, including the same preferred meanings, as described and defined herein above for the compound of formula (I).

In a 23^{rd} specific embodiment, the compound of formula (I) is a compound of formula (Ib) or a pharmaceutically acceptable salt or solvate thereof, wherein R¹ is isopropyl, wherein R^{A2} is hydrogen, and wherein R² has the same meanings, including the same preferred meanings, as described and defined herein above for the compound of formula (I).

In a 24^{th} specific embodiment, the compound of formula (I) is a compound of formula (Ib) or a pharmaceutically acceptable salt or solvate thereof, wherein R¹ is isopropyl, wherein R^{A2} is —CH₃, and wherein R² has the same meanings, including the same preferred meanings, as described and defined herein above for the compound of formula (I).

In a 25^{th} specific embodiment, the compound of formula (I) is a compound of formula (Ib) or a pharmaceutically acceptable salt or solvate thereof, wherein R¹ is isopropyl, wherein R^{A2} is —OCH₃, and wherein R² has the same meanings, including the same preferred meanings, as described and defined herein above for the compound of formula (I).

In a 26^{th} specific embodiment, the compound of formula (I) is a compound of formula (Ib) or a pharmaceutically acceptable salt or solvate thereof, wherein R¹ is ethyl, wherein R^{A2} is hydrogen, and wherein R² has the same meanings, including the same preferred meanings, as described and defined herein above for the compound of formula (I).

In a 27^{th} specific embodiment, the compound of formula (I) is a compound of formula (Ib) or a pharmaceutically acceptable salt or solvate thereof, wherein R¹ is ethyl, wherein R^{A2} is —CH₃, and wherein R² has the same meanings, including the same preferred meanings, as described and defined herein above for the compound of formula (I).

In a 28^{th} specific embodiment, the compound of formula (I) is a compound of formula (Ib) or a pharmaceutically acceptable salt or solvate thereof, wherein R¹ is ethyl, wherein R^{A2} is —OCH₃, and wherein 2 has the same meanings, including the same preferred meanings, as described and defined herein above for the compound of formula (I).

For a person skilled in the field of synthetic chemistry, various ways for the preparation of the compounds of formula (I), including also the above-discussed compounds of formulae (Ia) and (Ib), will be readily apparent. For example, the compounds of formula (I) can be prepared as illustrated in the following scheme, and as described in detail herein below:

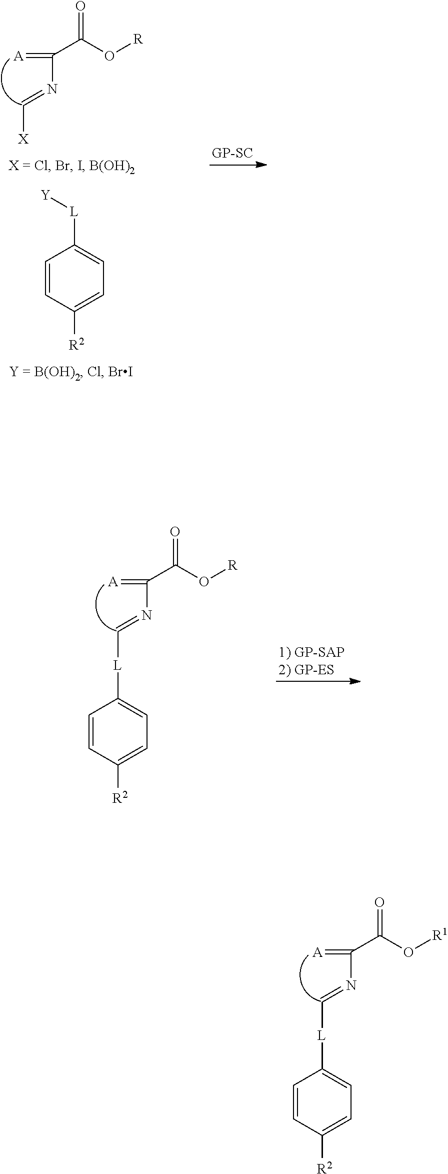

General Procedures for Suzuki Coupling GP-SC: SCA-SCB

SCA: In an inert Schlenk flask equipped with magnetic stirring bar 2-bromopyridine (resp. 2-bromothiazole) building block (1.0 eq), arylboronic acid (0.9 to 1.5 eq) and K₂CO₃ (2.0 eq) were dissolved in degassed abs. toluene (0.1 M). Pd[PPh₃]₄ (3 mol %) was added and the reaction mixture was stirred at 80° C. The reaction progress was monitored via TLC. When full conversion was observed, the reaction mixture was cooled down to RT and filtered through a pad of Celite. The solvent was removed under reduced pressure and the crude product was purified via column chromatography or preparative HPLC, respectively.

SCB: A Schlenk tube was dried under vacuum and charged with 1.0 eq halogenated substrate, 1.1 eq boronic acid, 5 mol % PdCl₂(dppf), 2.1 eq CsF, and anhydrous DME (˜5 mL/100 mg halogenated substrate). The mixture was degassed via three cycles of vacuum/inert gas and was stirred at 80° C. (oil bath) overnight, after which time the reaction mixture was cooled to rt and optionally filtered through a pad of silica gel or cotton. Subsequently, the solvent was removed under reduced pressure and final purification via column chromatography yielded the pure product. Reaction control was performed via TLC analysis and/or GC-MS analysis.

General Procedure Saponification (GP-SAP) SA1

SA1: A Schlenk tube was charged with the ester substrate and ˜10-20 mL MeOH/mmol substrate. Subsequently, 2.0-2.1 eq of a 2 M aqueous NaOH solution were added and the mixture was stirred overnight at 80-100° C. (oil-bath). The solvent was removed under reduced pressure and H₂O was added. The aqueous layer was optionally washed with CH₂Cl₂. Using conc. HCl, the aqueous layer was acidified to pH=1 and extracted exhaustively with EtOAc. Subsequently, the combined organic layers were dried over Na₂SO₄ or MgSO₄, filtered, and the solvent was removed under reduced pressure to give the pure product. Reaction control was performed via TLC analysis.

General Procedure for Esterification (GP-ES): EA-EC

EA (Fischer-Esterification): In a round-bottom flask heterocyclic acid (1.0 eq.) was dissolved in the corresponding alcohol (0.1-0.2 M) and H₂SO₄ (3.0 eq.) was added. The reaction mixture was equipped with an air condenser and stirred under reflux until full conversion was detected via TLC. The reaction mixture was cooled to RT and the solvent was removed under reduced pressure. The residue was taken up in satd. NaHCO₃ and extracted with CH₂Cl₂ (3×15 mL). The combined organic phase was dried over Na₂SO₄, filtered and the solvent was removed under reduced pressure. The crude product was used in the next step without further purification.

EB (DCC-mediated Esterification): In an inert 10 mL Schlenk flask heterocyclic acid (1.0 eq) was dissolved in CH₂Cl₂ abs. (0.1 M). N,N′-Dicyclohexylcarbodiimide (DCC) (1.5 eq.) and DMAP (0.2 eq.) were added successively and the reaction mixture was cooled to 0° C. using an ice bath. The corresponding alcohol (1.5 eq.) was added and the cloudy reaction mixture was stirred at RT until full conversion was observed via TLC. The reaction mixture was filtered through a pad of Celite and the solvent was removed under reduced pressure. The crude product was purified via column chromatography or preparative HPLC, respectively.

EC (EDC-mediated Esterification): A Schlenk tube was dried under vacuum and charged with 1.0 eq of the carboxylic acid substrate, anhydrous THE or CH₂Cl₂ (˜2 mL/100 mg carboxylic acid substrate), and 1.5 eq of the corresponding alcohol. Subsequently, 1.1 eq EDC*HCl (EDC: 1-ethyl-3-(3-dimethylaminopropyl)carbodiimide) and 0.15 eq DMAP were added at 0° C. (ice-bath) and the mixture was stirred at rt overnight. Subsequently, the mixture was filtered when necessary and the solvent was removed under reduced pressure and final purification via column chromatography yielded the pure product. Reaction control was performed via TLC analysis and/or GC-MS analysis.

Moreover, the compounds of formula (I) can also be prepared in accordance with, or in analogy to, the synthetic routes described in the examples section.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₅ alkenylene” denotes an alkenylene group having 2 to 5 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₅ alkynylene” denotes an alkynylene group having 2 to 5 carbon atoms. Unless defined otherwise, the term “alkynylene” preferably refers to C₂₋₄ alkynylene (including, in particular, linear C₂₋₄ alkynylene).

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. A heterocyclyl may be attached, e.g., via a ring carbon atom. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the aryl is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. A heteroaryl may be attached, e.g., via a ring carbon atom. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” ° preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. A heterocycloalkyl may be attached, e.g., via a ring carbon atom. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkeny” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. A heterocycloalkenyl may be attached, e.g., via a ring carbon atom. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. In this latter case, i.e. if all of the one or more halogen atoms are fluoro atoms, the corresponding haloalkyl group can also be referred to as a “fluoroalkyl” group. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint. If the term “about” is used in connection with a parameter that is quantified in integers, such as the number of nucleotides in a given nucleic acid, the numbers corresponding to ±10% or ±5% of the indicated numerical value are to be rounded to the nearest integer (using the tie-breaking rule “round half up”).

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. A preferred pharmaceutically acceptable salt of the compound of formula (I) is a hydrochloride salt.

Moreover, the scope of the invention embraces the compounds of formula (I) in any solvated form, including, e.g., solvates with water (i.e., as a hydrate) or solvates with organic solvents such as, e.g. methanol, ethanol or acetonitrile (i.e., as a methanolate, ethanolate or acetonitrilate). All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al, J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds provided herein may be administered as compounds per se or may be formulated as medicaments (pharmaceutical compositions). The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a no-ionic surfactant, tyloxapol, polysorbate 80, macrogol-15-hydroxystearate (e.g., Kolliphor© HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which is also referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are oral administration or parenteral administration. Even more preferably, the compounds or pharmaceutical compositions provided herein are to be administered orally.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or a pharmaceutical composition comprising the compound of formula (I) can be administered in monotherapy (e.g., without concomitantly administering any further therapeutic agents, or without concomitantly administering any further therapeutic agents against the same disease that is to be treated or prevented with the compound of formula (I)). However, the compound of formula (I) or a pharmaceutical composition comprising the compound of formula (I) can also be administered in combination with one or more further therapeutic agents. If the compound of formula (I) is used in combination with a second therapeutic agent active against the same disease or condition, the dose of each compound may differ from that when the corresponding compound is used alone, in particular, a lower dose of each compound may be used. The combination of the compound of formula (I) with one or more further therapeutic agents may comprise the simultaneous/concomitant administration of the compound of formula (I) and the further therapeutic agent(s) (either in a single pharmaceutical formulation or in separate pharmaceutical formulations), or the sequential/separate administration of the compound of formula (I) and the further therapeutic agent(s). If administration is sequential, either the compound of formula (I) according to the invention or the one or more further therapeutic agents may be administered first. If administration is simultaneous, the one or more further therapeutic agents may be included in the same pharmaceutical formulation as the compound of formula (I), or they may be administered in two or more different (separate) pharmaceutical formulations.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease, as used herein, is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention is also described by the following illustrative figures. The appended figures show:

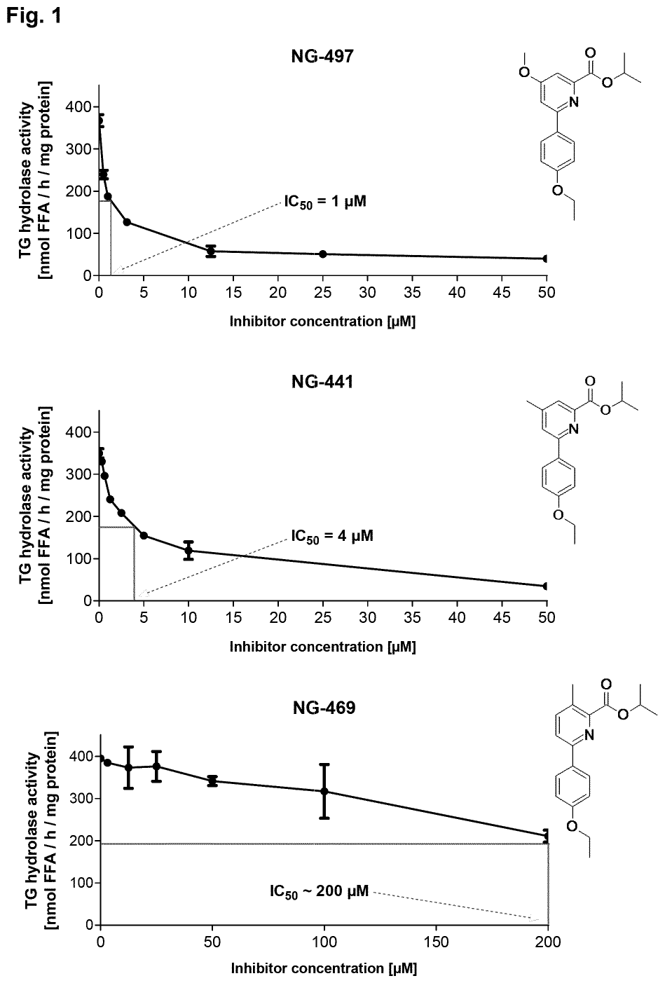

FIG. 1: Illustrative examples for the determination of IC₅₀ values for human ATGL, revealing IC₅₀ values of 1 μM for Example 35/NG-497 (A), 4 μM for Example 20/NG-441 (B), and >200 μM for the reference compound NG-469 (C). See Example 237.

FIG. 2: (A) Cross-species inhibitory activity of various compounds of formula (I) as well as atglistatin (each at 50 μM) on human ATGL, macaque ATGL, murine ATGL, and rat ATGL. (B) Cross-species inhibitory activity of Example 152/Tsch-62A (at 50 μM) on human ATGL, macaque ATGL, murine ATGL, and rat ATGL. See Example 237.

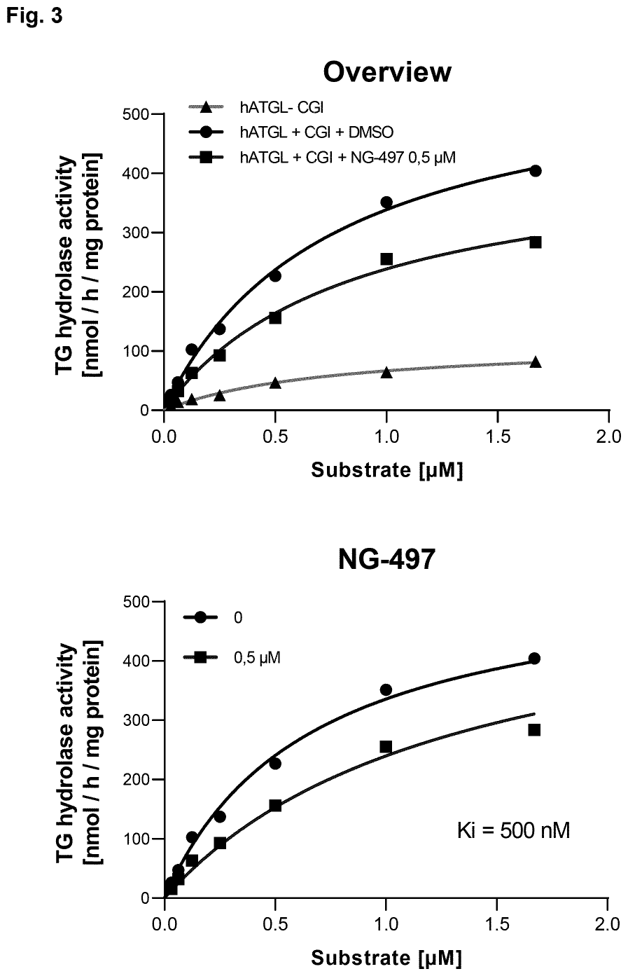

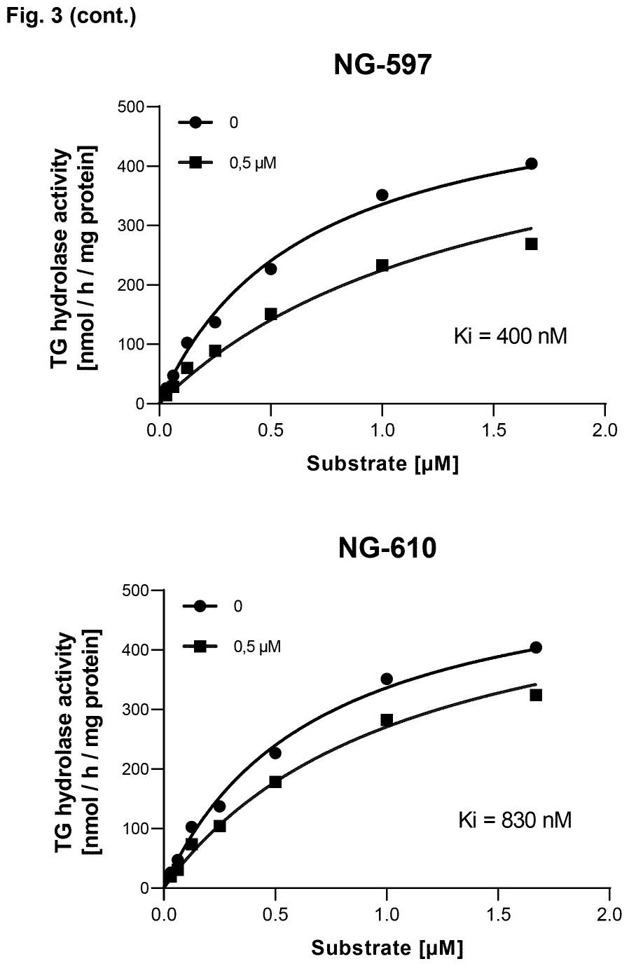

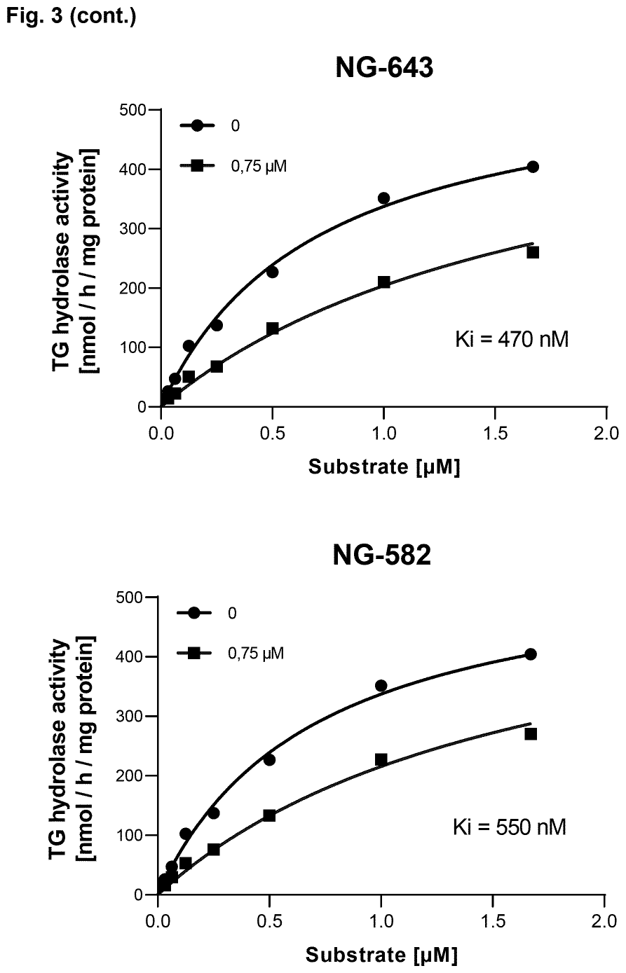

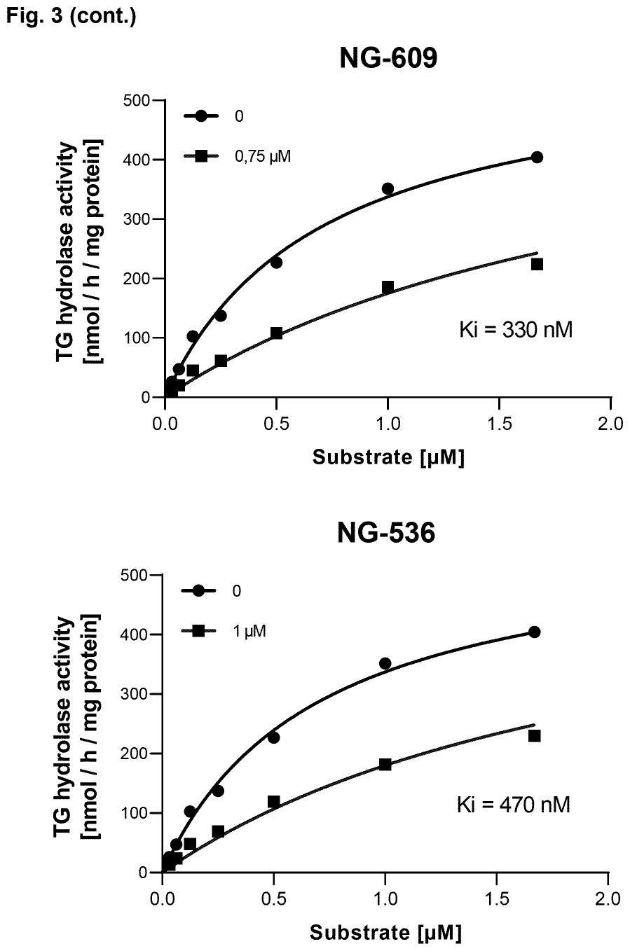

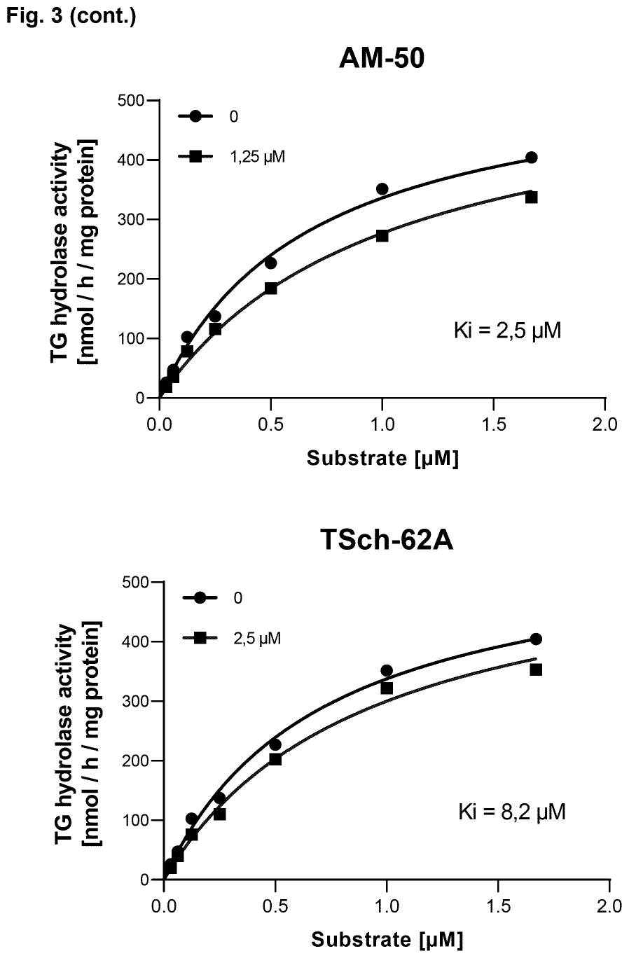

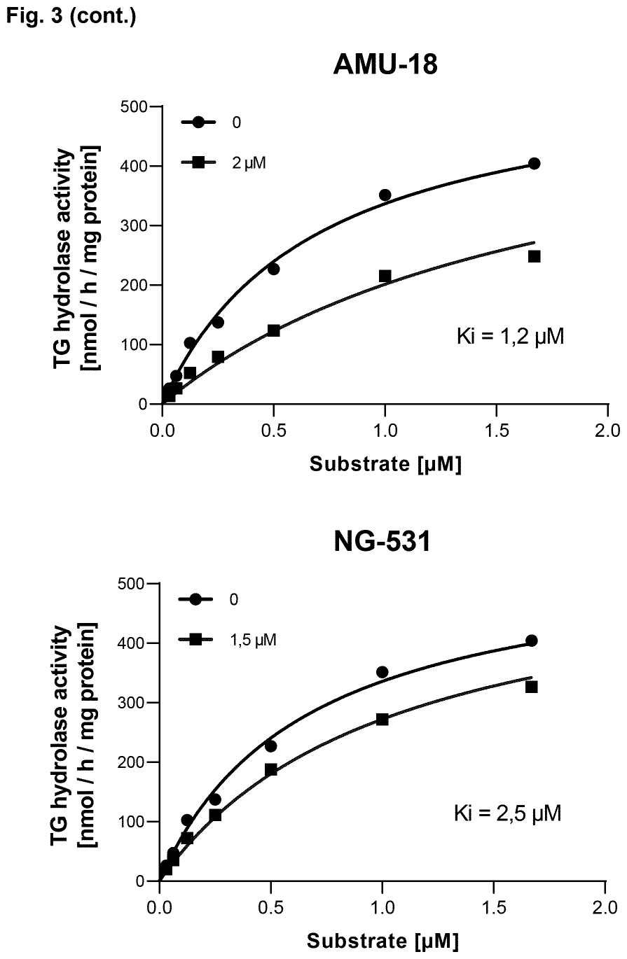

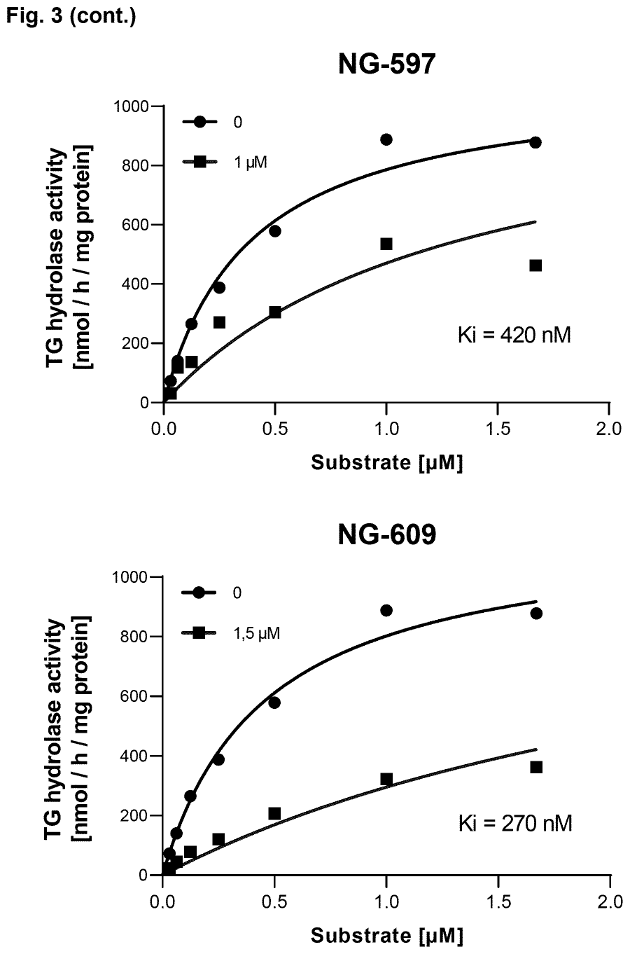

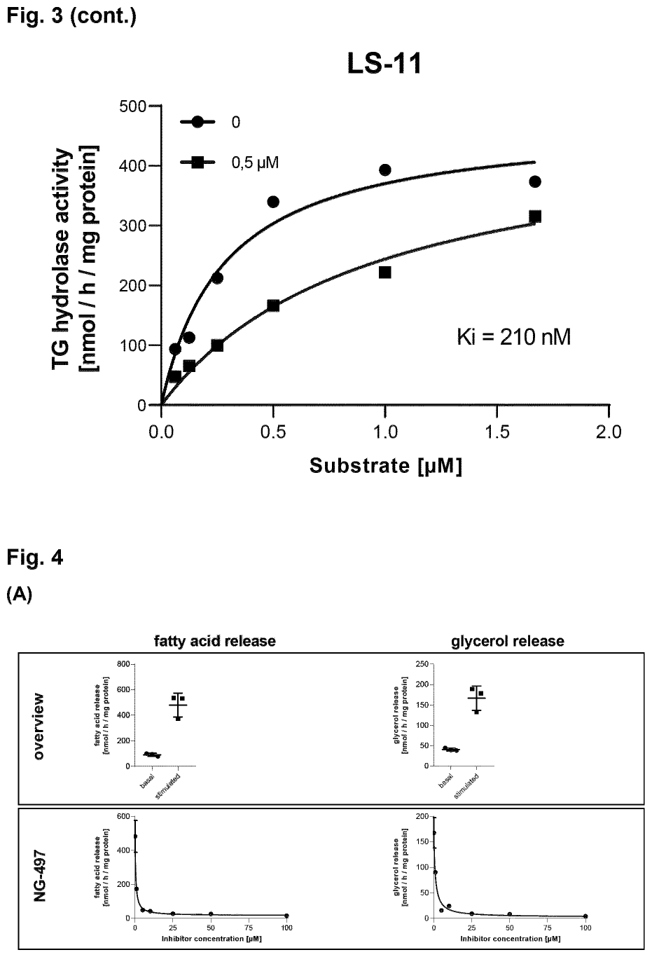

FIG. 3: Determination of Ki values of various compounds of formula (I). See Example 238.

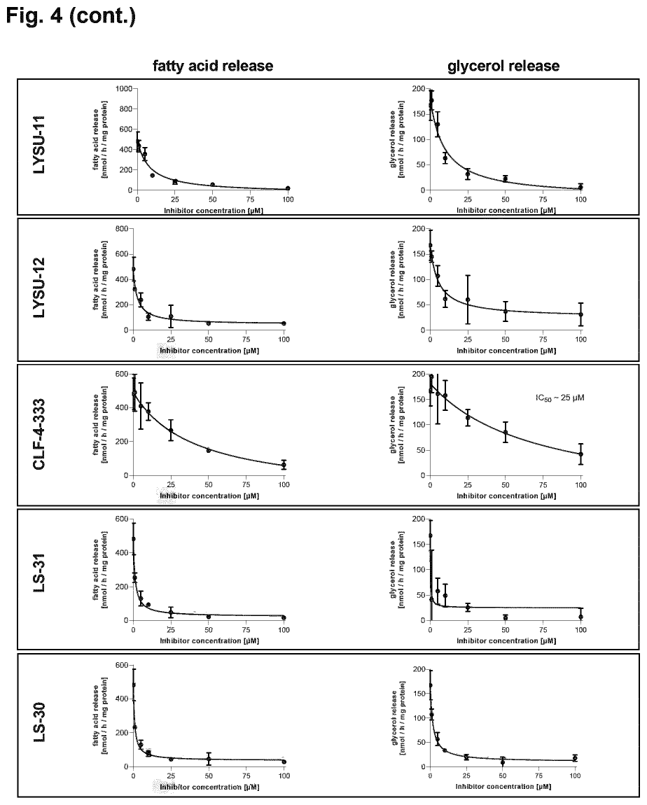

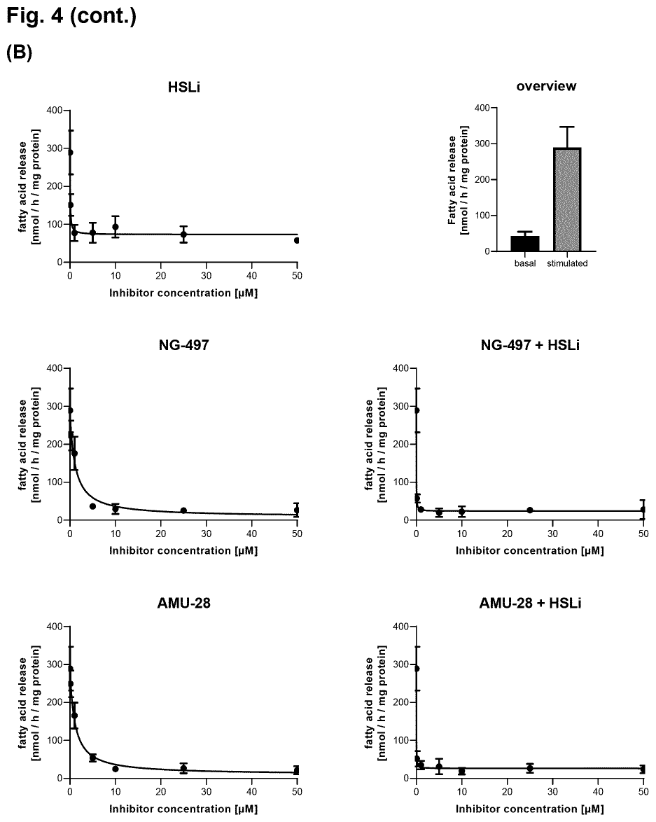

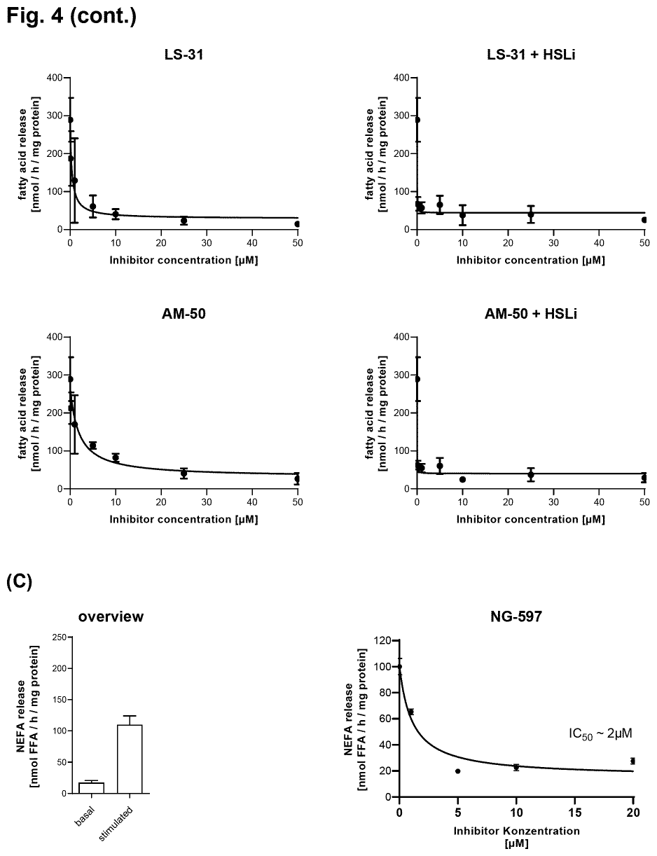

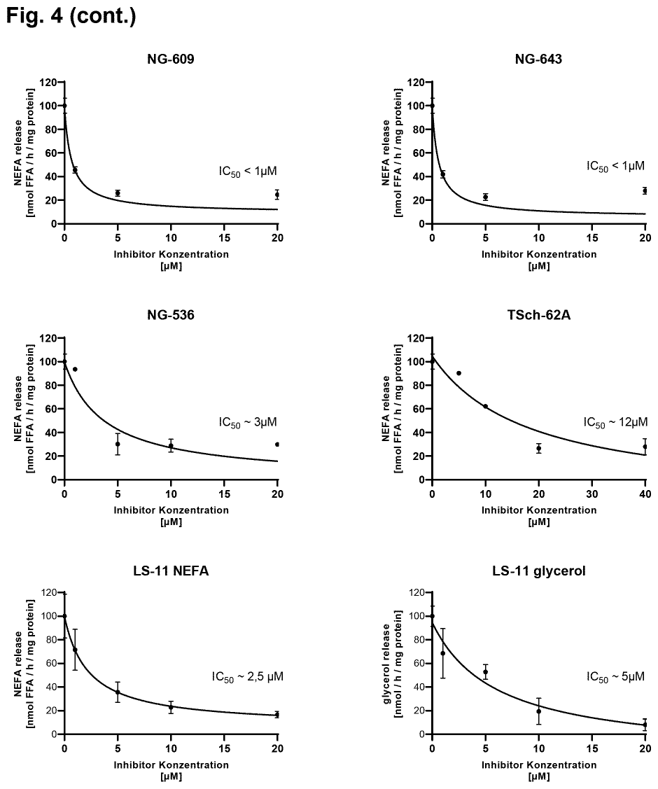

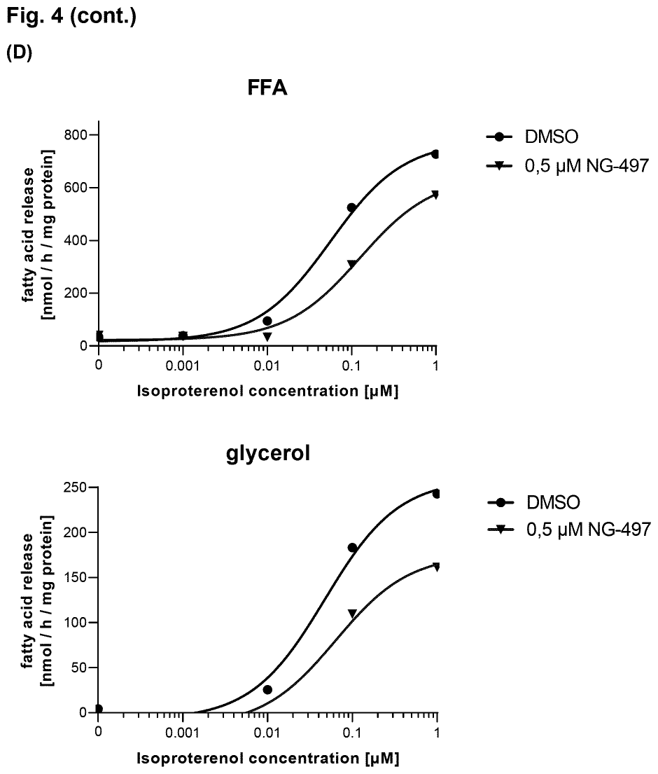

FIG. 4: Inhibition of fatty acid release from human adipocytes. (A) Effects of cross-species ATGL inhibitors on isoproterenol stimulated fatty acid and glycerol release from differentiated human adipocytes (SGBS). (B) Differentiated SGBS adipocytes were preincubated with inhibitors —/+25 μM HSL inhibitor for 2 h and fatty acid release was stimulated by DMEM containing 2% FA-free BSA and 1 μM isoproterenol. FA concentration in the medium was determined after 1 h via Wako Diagnostics NEFA reagent. Samples were measured in triplicates. (C) Effects of ATGL inhibitors on isoproterenol stimulated fatty acid and glycerol release from differentiated human adipocytes (hMADS). (D) Inhibition of ATGL upon submaximal stimulation of lipolysis. Human differentiated SGBS adipocytes were preincubated with NG-497 (0.5 μM) or DMSO for 2 h. Subsequently, lipolysis was stimulated with different concentrations of isoproterenol for 1 h. Fatty acid and glycerol release was determined from media. Data are presented as mean, samples were measured in triplicates. See Example 238.

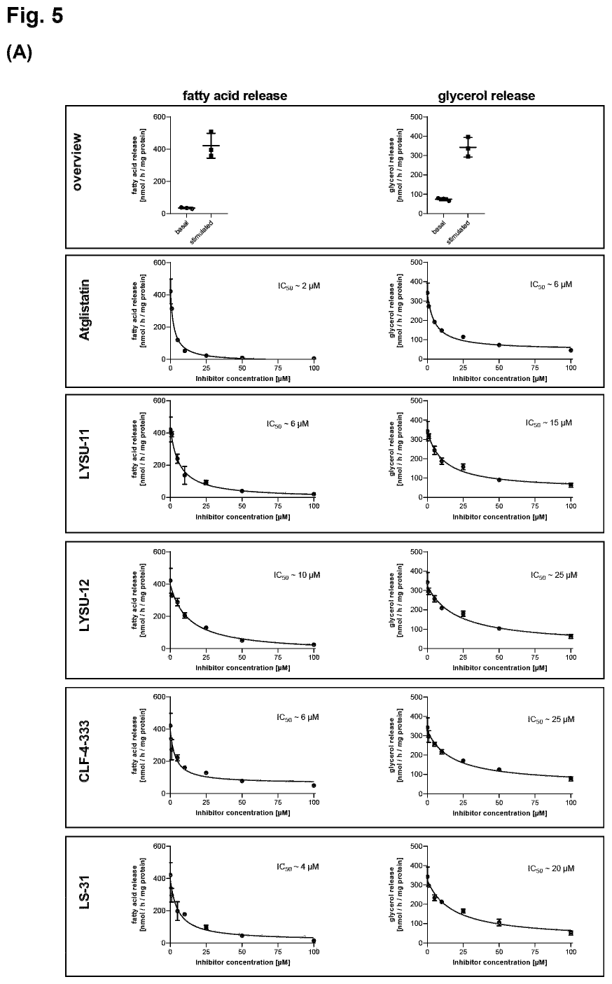

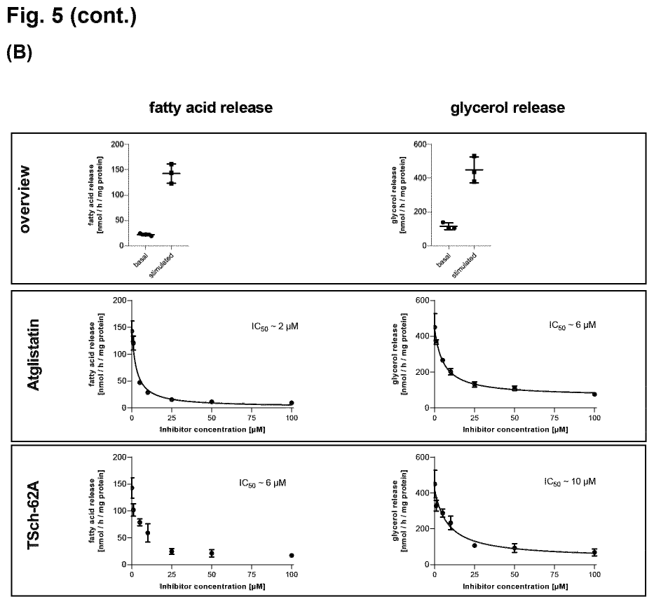

FIG. 5: Inhibition of fatty acid release from murine adipocytes. (A) Effects of cross-species ATGL inhibitors on isoproterenol stimulated fatty acid and glycerol release from differentiated murine adipocytes (3T3-L1). (B) Effects of dual human/murine ATGL inhibitor TSch-62A on isoproterenol stimulated fatty acid and glycerol release from differentiated murine adipocytes (3T3-L1). See Example 238.

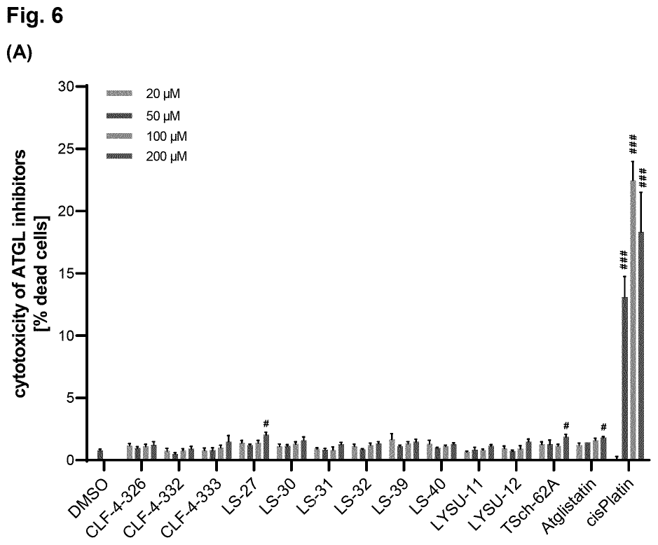

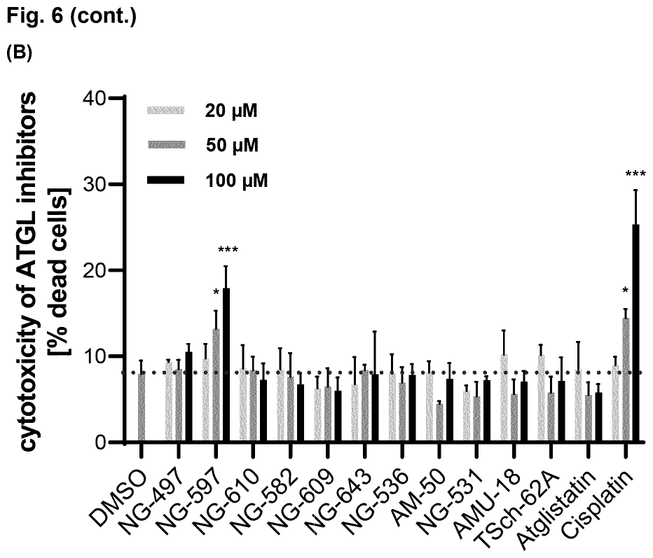

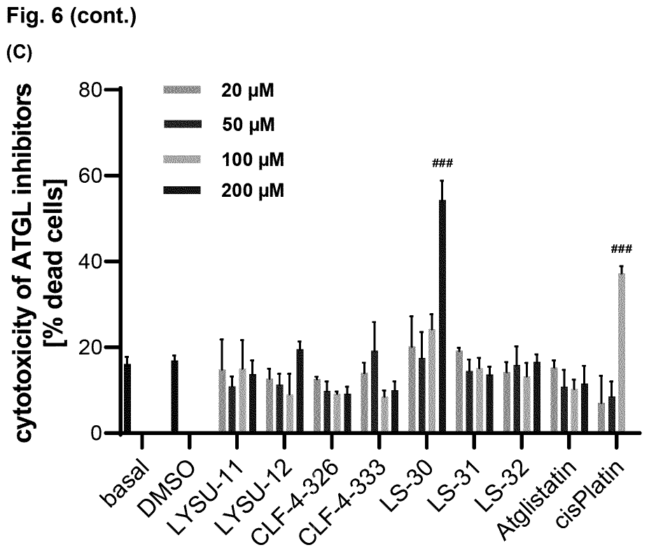

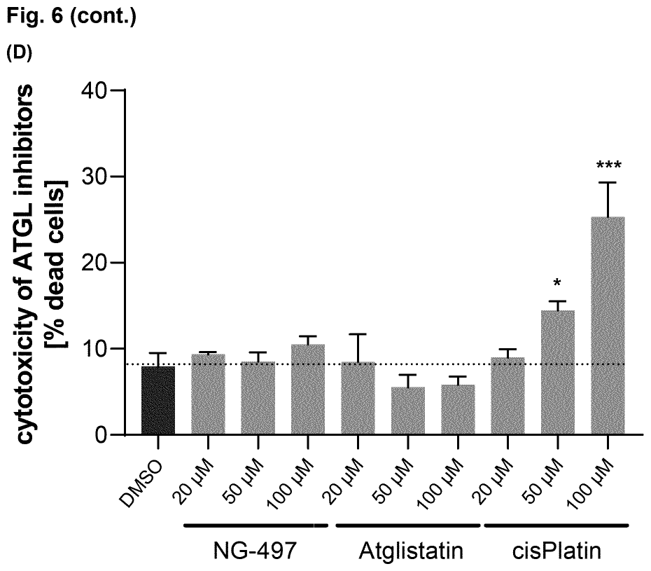

FIG. 6: Toxicity screening (LDH based) in HepG2 cells. (A) Toxicity of cross-species ATGL inhibitors in human liver cells. HepG2 cells were seeded in 96 well plates and at 80% confluency treated with DMSO (0.5% final conc.) or ATGL inhibitors for 24 h in DMEM+P/S+3% heat inactivated FCS (3 h at 62° C.). Subsequently, LDH activity of 50 μl supernatant was determined via the Roche LDH Kit. Samples measured in triplicates and represented as mean+S.D. Statistical significance was determined via 2-way ANOVA and Dunnett's post hoc test. #p<0.05, ##p<0.01, and ###p<0.001 vs. DMSO control. (B) Toxicity of hATGL inhibitors. HepG2 cells were seeded in 96 well plates and at 80% confluency treated with DMSO (0.5% final conc.) or hATGL inhibitors for 24 h in DMEM+P/S+10% heat inactivated FCS (3 h at 62° C.). Subsequently, medium was centrifuged at 300 g for 3 min, and LDH activity of 50 μl supernatant was determined via the Roche LDH Kit. Samples measured in triplicates. Statistical significance was determined via ANOVA and Dunnett's post hoc test. (C) Toxicity of hATGL inhibitors. HepG2 cells were seeded in 96 well plates and at 50% confluency treated with DMSO (0.5% final conc.) or hATGL inhibitors for 24 h in DMEM+P/S+3% heat inactivated FCS (3 h at 62° C.). Subsequently, LDH activity of 50 μl medium was determined via the Roche LDH Kit. Samples measured in triplicates. Statistical significance was determined via ANOVA and Dunnett's post hoc test. ###p<0.001 vs. DMSO controls. (D) Toxicity of human ATGL inhibitor NG-497. HepG2 cells were treated with DMSO (0.5% final conc.) or NG-497 for 24 h in DMEM+P/S+10% heat inactivated FCS. Atglistatin was used as negative and cisPlatin as positive control. Subsequently, medium was centrifuged and LDH activity of the supernatant was determined via the Roche LDH Kit. Samples measured in triplicates. Cytotoxicity was calculated as relative amount of released LDH as compared to fully lysed cells. Samples measured in triplicates. Statistical significance was determined via ANOVA followed by Dunnett's post hoc test in respect to DMSO control. See Example 238.

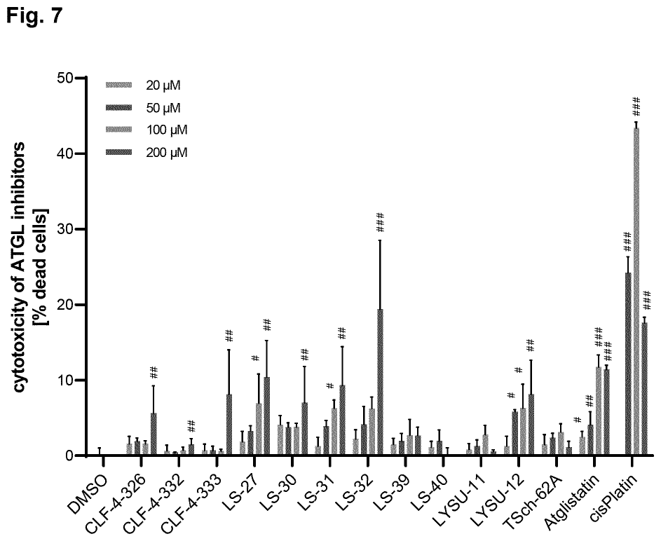

FIG. 7: Toxicity screening (LDH based) in AML-12 cells. Toxicity of cross-species ATGL inhibitors in murine liver cells. AML-12 cells were seeded in 96 well plates and at 80% confluency treated with DMSO (0.5% final conc.) or ATGL inhibitors for 24 h in DMEM+P/S+3% heat inactivated FOS (3 h at 62° C.). Subsequently, medium was centrifuged at 300 g for 3 min, and LDH activity of 50 μl supernatant was determined via the Roche LDH Kit. Samples measured in triplicates. Statistical significance was determined via ANOVA and Dunnett's post hoc test. See Example 238.

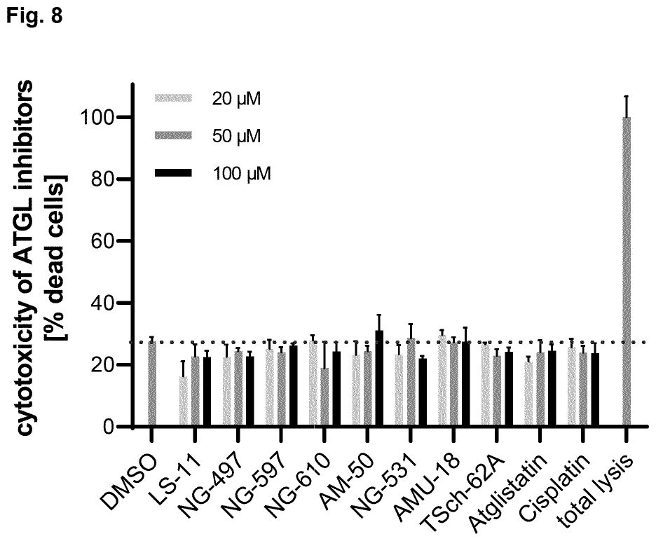

FIG. 8: Toxicity screening (LDH based) in PBMCs. PBMC toxicity of hATGL inhibitors. Human primary macrophages from MUG (Sabine Wagner) were seeded in 96 well plates treated with DMSO (0.25% final conc.) or hATGL inhibitors for 24 h in RPMI+P/S+5% heat inactivated FCS (3 h at 62° C.). Subsequently, LDH activity of 10 μl supernatant was determined via the Roche LDH Kit. Samples measured in triplicates. See Example 238.

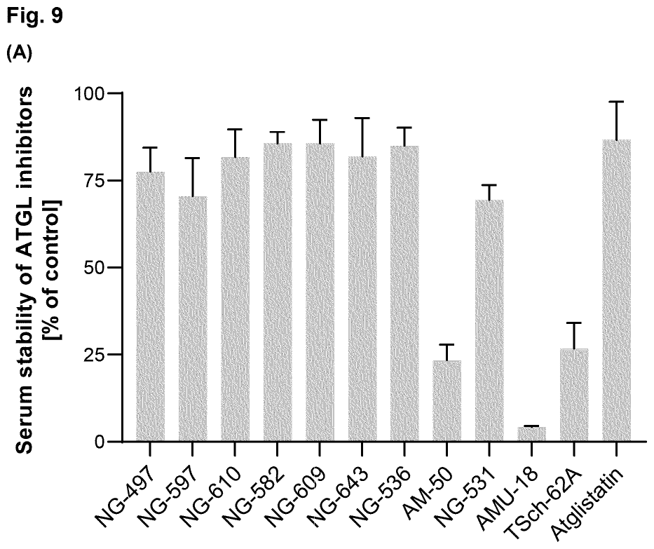

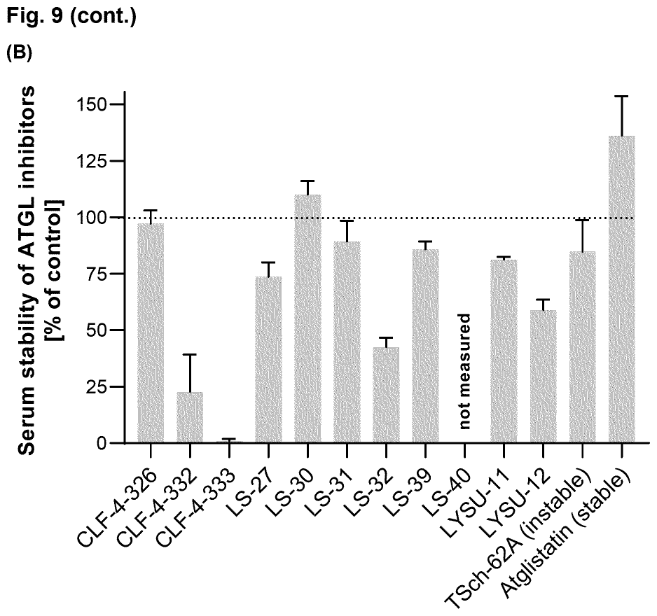

FIG. 9: Stability of hATGL inhibitors in human serum. (A) Inhibitors were incubated in human serum for 0 or 3 h at 37° C. at a concentration of 50 μM, subsequently extracted with the MTBE method and analyzed via HPLC MS. Samples measured in triplicates. (B) Inhibitors were incubated in human serum for 0 or 3 h at 37° C. and subsequently extracted with the MTBE method and analyzed via HPLC MS. Samples measured in triplicates. See Example 238.

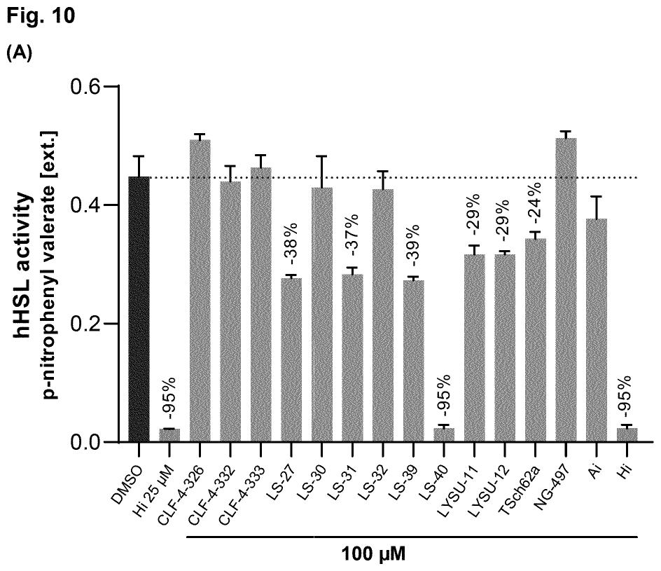

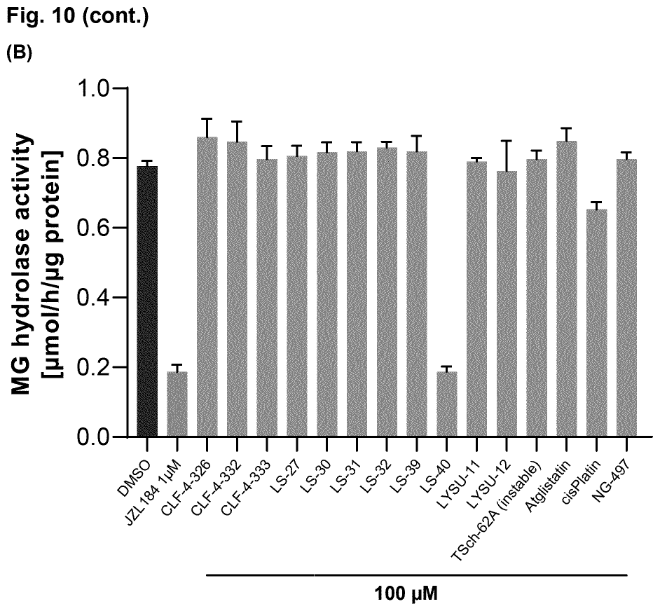

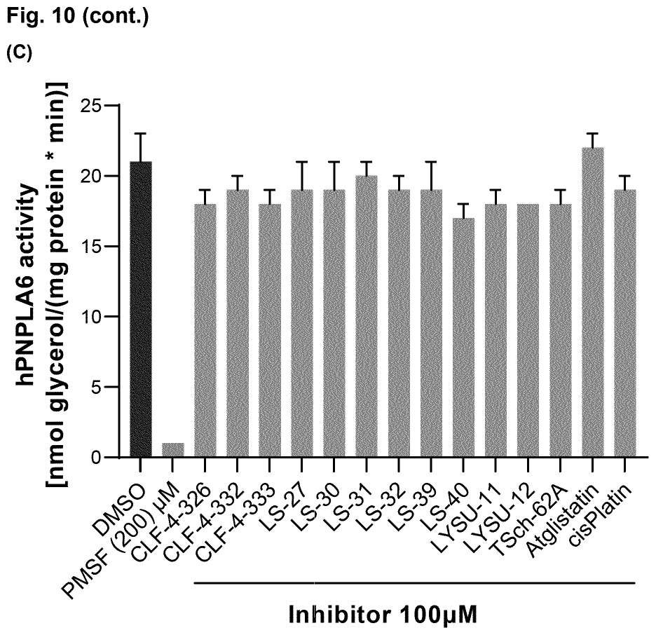

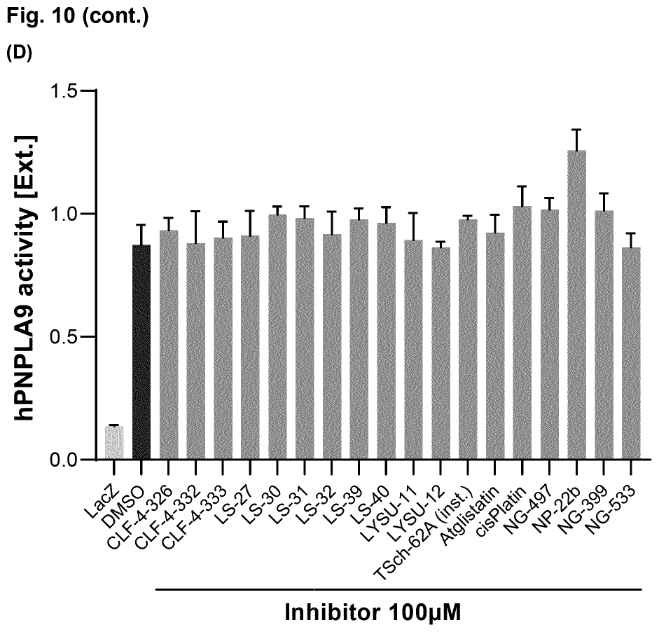

FIG. 10: Off-target inhibition. (A) Effects of pan-species ATGL inhibitors on hHSL activity. Expi lysates expressing hHSL (125 μl) were preincubated with 100 μM inhibitors for 30 min and incubated with 1 mM pNV substrate (100 μl) for 30 in. Final concentration 440 μM pNV. Samples measured in triplicates. (B) Effects of pan-species ATGL inhibitors on MG hydrolysis activity of mMGL. Lysates from E. coli expressing mMGL were treated with 100 μM inhibitors and incubated with 1 mM rac-OG substrate for 10 min. Enzyme activity was measured using the Free Glycerol Reagent. Samples measured in triplicates. (C) Effects of pan-species ATGL inhibitors on hPNPLA6 activity. Expi lysates expressing hPNPLA6 were treated with 100 μM inhibitors and incubated with 1 mM LPC substrate for 30 min. Samples measured in triplicates. (D) Effects of pan-species ATGL inhibitors on hPNPLA9 activity. Expi lysates expressing hPNPLA9 were treated with 100 μM inhibitors. Samples measured in triplicates. See Example 238.

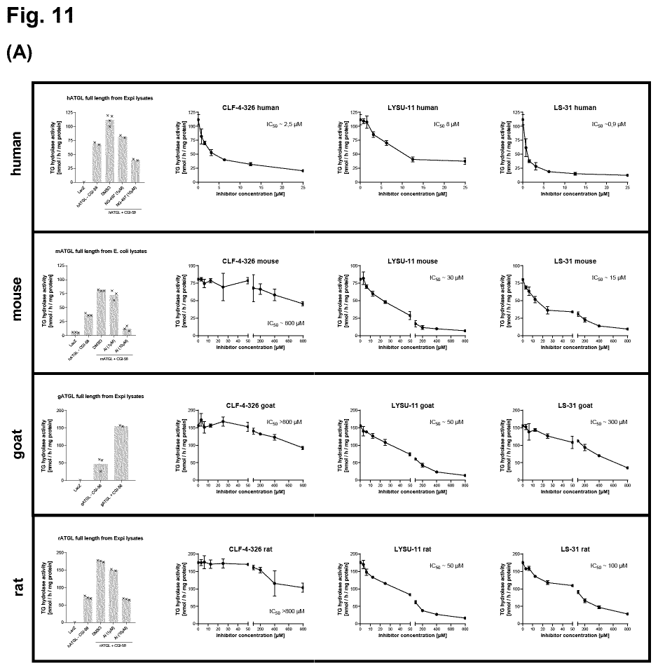

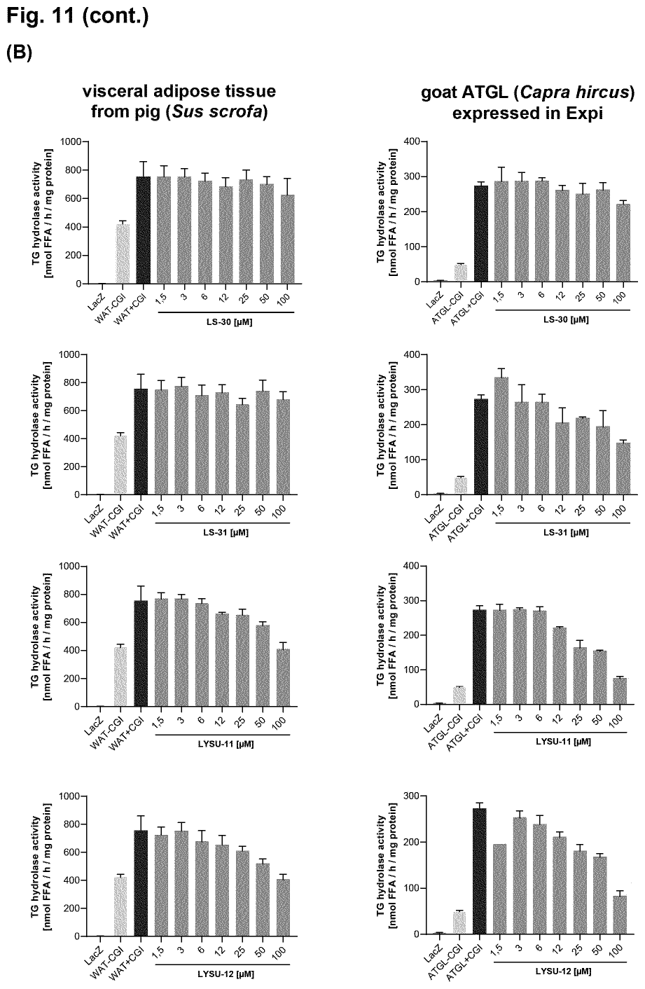

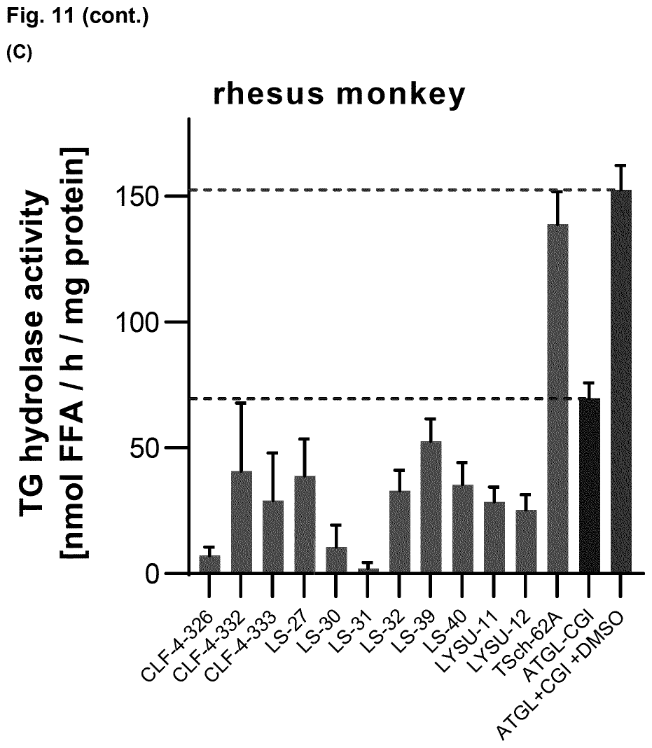

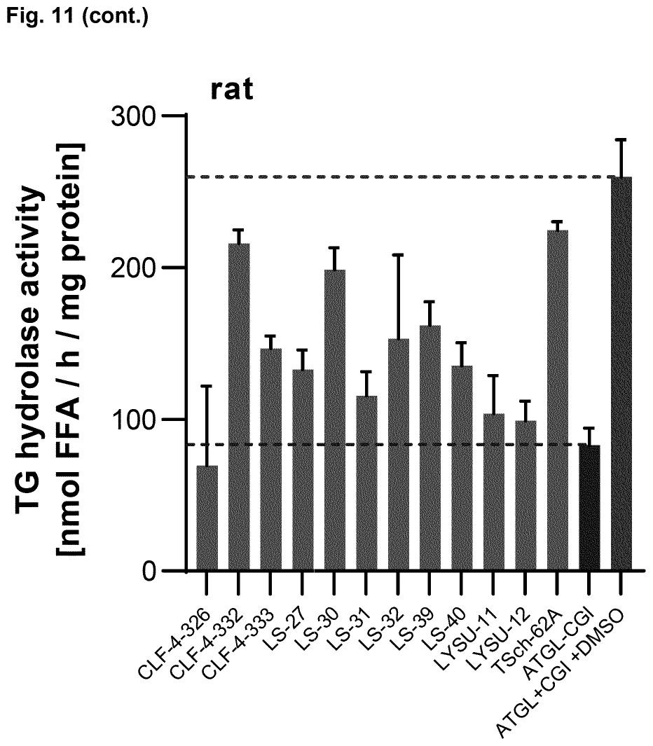

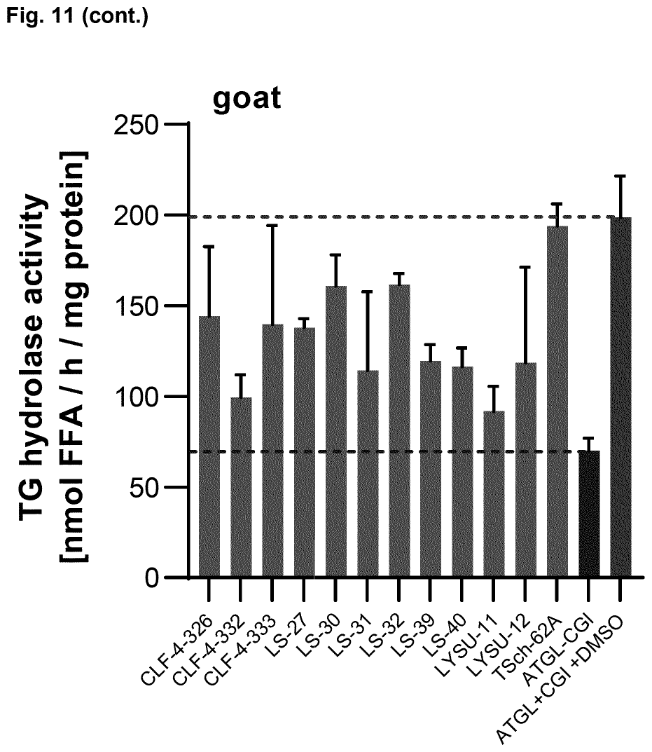

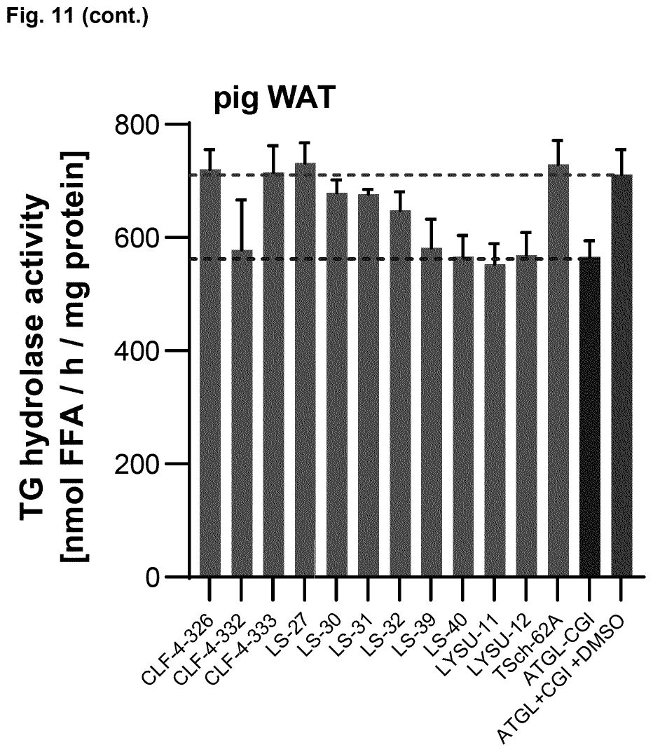

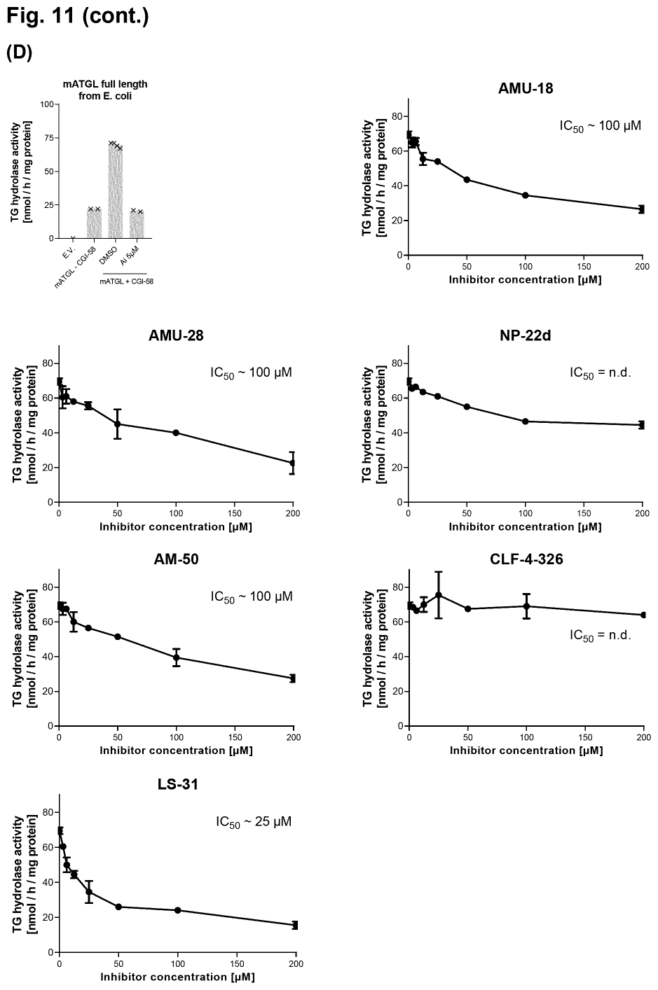

FIG. 11: Cross-species reactivity. (A) Inhibition of in vitro TG hydrolase activity by cross-species ATGL inhibitors. ATGL from different species were expressed in Expi cells, lysates were stimulated with purified CGI-58 and TG hydrolase activity determined via 3H labelled triolein. FA release was determined via liquid scintillation. (B) Effects of cross-species ATGL inhibitors on mCGI-58 stimulated TG hydrolase activity of vWAT from pig and Expi lysates expressing goat ATGL. Samples measured in triplicates. (C) Inhibition of in vitro TG hydrolase activity by cross-species ATGL inhibitors. ATGL from different species were expressed in Expi cells, lysates were stimulated with purified CGI-58 and TG hydrolase activity determined in presence of 50 μM ATGL inhibitors via 3H labelled triolein. FA release was determined via liquid scintillation. (D) Inhibition of mouse ATGL by hATGL inhibitors. ATGL from mouse (Mus musculus) was expressed in Expi cells, lysates were stimulated with purified CGI-58 and TG hydrolase activity determined via 3H labelled triolein. FA release was determined via liquid scintillation. See Example 238.

The invention will now be described by reference to the following examples which are merely illustrative and are not to be construed as a limitation of the scope of the present invention.

### EXAMPLES

Various compounds described in this section are defined by their chemical formulae and their corresponding chemical names. In case of conflict between any chemical formula and the corresponding chemical name indicated herein, the present invention relates to both the compound defined by the chemical formula and the compound defined by the chemical name, and particularly relates to the compound defined by the chemical formula.

### Examples 1 to 97

General Information

Reactions were carried out under air, unless indicated otherwise. For inert reactions, standard Schlenk techniques under an inert atmosphere of N₂ or Ar and anhydrous solvents were used. Specific rotation was measured at 20° C. with a wavelength of 589 nm with a Perkin Elmer Polarimeter 341. The described nuclear resonance spectra were acquired with the following instruments: Bruker AVANCE III with Autosampler: 300.36 MHz ¹H-NMR, 75.53 MHz ¹³C-NMR; Varian Unity Inova: 499.91 MHz ¹H-NMR, 125.69 MHz ¹³C-NMR, 470.35 MHz ¹⁹F-NMR. Chemical shifts δ [ppm] are referenced to residual protonated solvent signals as internal standard: CDCl₃: δ=7.26 ppm (¹H), 77.16 ppm (¹³C), DMSO-d₆: δ=2.50 ppm (¹H), 39.52 ppm (¹³C), and MeOD-d₄: δ=3.31 ppm (¹H), 49.00 ppm (¹³C). Signal multiplicities are abbreviated as bs (broad singlet), d (dublet), dd (doublet of doublet), dq (doublet of quadruplet), dt (doublet of triplet), hept (heptett), m (multiplet), s (singlet), t (triplet), and q (quadruplet). The deuterated solvent, the chemical shifts δ in ppm (parts per million), and the coupling constants J in Hertz (Hz) are given. Deuterated solvents for nuclear resonance spectroscopy were purchased from Euriso Top® (CDCl₃, MeOD-d₄) and Aldrich® (DMSO-d₆). Data analysis was performed using the software “MestreNova”. An automatic phase correction as well as an automated baseline correction (Whittaker Smoother) were performed for several spectra. Analytical thin layer chromatography (TLC) was performed on Merck silica gel 60 F254 plates and spots were visualized by UV-light (A=254 and/or 366 nm), or by treatment with KMnO₄ solution (3.0 g KMnO₄ and 20.0 g K₂CO₃ dissolved in 300 mL of a 5% NaOH solution). Column chromatography was performed using silica gel 60 Å (0.04-0.063 mm particle size) from Macherey-Nagel. High Resolution Mass Spectrometry (HRMS): TOF MS EI was performed on a Waters GCT premier micromass with an Electron Impact Ionization (EI)-source (70 eV) and samples were injected via direct insertion (DI). Melting points were determined on a Mel Temp® melting point apparatus (Electrothermal). Purifications via preparative HPLC were performed on a Dionex UltiMate 3000. The separation was carried out using a C-18 reversed-phase column of the type “Nucleodur®100-5” by Macherey-Nagel at 30° C., and detection was accomplished at a wavelength of A=210 nm. Three different methods were used: “method A”: 0-3 min 98% of a 0.01% aqueous formic acid solution and 2% CH₃CN, 3-15 min linear to 100% CH₃CN, 15-18 min 100% CH₃CN with a flow of 15 mLmin⁻¹; “method B”: 0-3 min 98% of H₂O and 2% CH₃CN, 3-15 min linear to 100% CH₃CN, 15-18 min 100% CH₃CN with a flow of 15 mLmin⁻¹; “method C”: 0-2 min 90% of H₂O and 10% CH₃CN, 2-12 min linear to 100% CH₃CN, 12-14 min 100% CH₃CN with a flow of 15 mLmin¹. High pressure hydrogenation experiments were performed using the H-Cube™ continuous hydrogenation unit (HC-2.SS) from Thales Nanotechnology Inc. running with a Knauer Smartline pump 100 and equipped with a 10 mL ceramic pump head. As hydrogenation catalyst 10% Pd/C catalyst cartridges were used (Thales Nanotechnology inc., THS01111, 10% Pd/C CatCart™). Chemicals were purchased mainly from the companies ABCR, ACROS Organics, Alfa Aesar, Sigma Aldrich or TCI and were used without further purification, unless stated otherwise. For inert reactions, solvents were stored under an argon atmosphere, and stored over molecular sieves (4 Å molecular sieves were used for CH₂Cl₂, 1,4-dioxane, DME, DMF, DMSO, Et₃N, pyridine, and THF. 3 Å molecular sieves were used for ACN and EtOH). The following solvents were additionally dried and distilled under an argon atmosphere: CH₂Cl₂(CaH₂), Et₃N (Na), EtOH (Na), THF (CaH₂). ACN for inert reactions was passed through an aluminium oxide column (solvent purification system: Puresolv™ from Innovative Technology Inc.) under inert conditions.

General Procedures Equivalents of reagents and catalysts may vary by +/−5% or +/−1 mol % compared to the given values.

General Procedure Suzuki Coupling SC1

A Schienk tube was dried under vacuum and charged with 1.0 eq halogenated substrate, 1.1 eq boronic acid, 5 mol % PdCl₂(dppf), 2.1 eq CsF, and anhydrous DME (˜5 mL/100 mg halogenated substrate). The mixture was degassed via three cycles of vacuum/inert gas and was stirred at 80° C. (oil-bath) overnight, after which time the reaction mixture was cooled to rt and optionally filtered through a pad of silica gel or cotton. Subsequently, the solvent was removed under reduced pressure and final purification via column chromatography yielded the pure product. Reaction control was performed via TLC analysis and/or GC-MS analysis.

General Procedure Suzuki Coupling SC2

A Schlenk tube was dried under vacuum and charged with 1.0 eq halogenated substrate, 1.1 eq boronic acid, 5 mol % PdCl₂(dppf), 2.1 eq CsF, and anhydrous DME (˜5 mL/100 mg halogenated substrate). The mixture was degassed by three cycles of vacuum/inert gas and was stirred at 80° C. (oil-bath) overnight, after which time additional 0.3 eq boronic acid and 3 mol % PdCl₂(dppf) were added. Subsequently, the reaction mixture was stirred at 80° C. (oil-bath) overnight and was then cooled to rt and optionally filtered through a pad of silica gel or cotton. After solvent removal under reduced pressure, the crude product was purified via column chromatography. Reaction control was performed via TLC analysis and/or GC-MS analysis.

General Procedure Suzuki Coupling SC3

A Schlenk tube was dried under vacuum and charged with 1.0 eq halogenated substrate, 1.0 eq boronic acid, 5 mol % PdCl₂(dppf), 2.1 eq CsF, and anhydrous DME (˜5 mL/100 mg halogenated substrate). The mixture was degassed via three cycles of vacuum/inert gas and was stirred at 80° C. (oil-bath) overnight, after which time the reaction mixture was cooled to rt and optionally filtered through a pad of silica gel or cotton. Subsequently, the solvent was removed under reduced pressure and final purification via column chromatography yielded the pure product. Reaction control was performed via TLC analysis and/or GC-MS analysis.

General Procedure Esterification ES1

A Schienk tube was dried under vacuum and charged with 1.0 eq of the carboxylic acid substrate, anhydrous THF (˜2 mL/100 mg carboxylic acid substrate), and 1.5 eq of the corresponding alcohol. Subsequently, 1.1 eq EDC*HCl and 0.15 eq DMAP were added at 0° C. (ice-bath) and the mixture was stirred at rt overnight. Subsequently, the mixture was filtered when necessary and the solvent was removed under reduced pressure and final purification via column chromatography yielded the pure product. Reaction control was performed via TLC analysis and/or GC-MS analysis.

General Procedure Esterification ES2

A Schienk tube was dried under vacuum and charged with 1.0 eq of the carboxylic acid substrate, anhydrous CH₂Cl₂, 3.0 eq EDC*HCl, 0.3 eq DMAP, and 3.1 eq of the corresponding alcohol. The mixture was stirred at rt overnight or over the weekend, the solvent was removed under reduced pressure, and final purification via column chromatography yielded the pure product. Reaction control was performed via TLC analysis.

General Procedure Esterification ES3

A Schlenk tube was dried under vacuum and charged with 1.0 eq of the carboxylic acid substrate, anhydrous THF (˜2 mL/100 mg carboxylic acid substrate), and 1.0 eq of the corresponding alcohol. Subsequently, 1.0 eq EDC*HCl and 0.1 eq DMAP were added at 0° C. (ice-bath) and the mixture was stirred at rt overnight. Subsequently, the mixture was filtered when necessary and the solvent was removed under reduced pressure and final purification via column chromatography yielded the pure product. Reaction control was performed via TLC analysis and/or GC-MS analysis.

General Procedure Saponification SA1

A Schlenk tube was charged with the ester substrate and ˜10-20 mL MeOH/mmol substrate. Subsequently, 2.0-2.1 eq of a 2 M aqueous NaOH solution were added and the mixture was stirred overnight at 80-100° C. (oil-bath). The solvent was removed under reduced pressure and H₂O was added. The aqueous layer was optionally washed with CH₂Cl₂. Using 37m % HCl, the aqueous layer was acidified to pH=1 and extracted several times with EtOAc. Subsequently, the combined organic layers were dried over Na₂SO₄ or MgSO₄, filtered, and the solvent was removed under reduced pressure to give the pure product. Reaction control was performed via TLC analysis.

General Procedure Saponification SA2

A Schlenk tube was charged with the ester substrate and ˜7 mL MeOH/mmol substrate. Subsequently, 1.55 eq of a 2 M aqueous NaOH solution were added and the mixture was stirred overnight at 80° C. (oil-bath). The solvent was removed under reduced pressure and H₂O was added. The aqueous layer was acidified to pH=1 with 37 m % HCl and extracted several times with EtOAc. Subsequently, the combined organic layers were dried over Na₂SO₄, filtered, and the solvent was removed under reduced pressure to give the pure product. Reaction control was performed via TLC analysis.

Experimental Procedures

### Example 1: NG-442

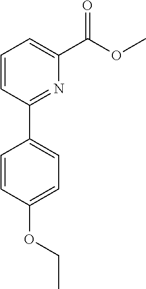 [SMILES: CCOc1ccc(-c2cccc(C(=O)OC)n2)cc1]

The coupling of methyl 6-bromopyridine-2-carboxylate with 4-ethoxyphenylboronic acid was performed following the general procedure SC1 with the modification that 1.0 g of the bromine substrate were dissolved in 25 mL DME.

Yield=1.086 g yellowish solid (4.22 mmol, 91%).

R_{f}=0.34 (cyclohexane/EtOAc=4+1; UV).

¹H-NMR (300 MHz, CDCl₃): δ=8.08-7.90 (m, 3H), 7.89-7.75 (m, 2H), 6.98 (d, J=8.6 Hz, 2H), 4.17-3.90 (m, 5H), 1.52-1.32 (t, J=6.87 Hz, 3H).

¹³C-NMR,APT (76 MHz, CDCl₃): δ=166.2, 160.4, 157.5, 148.0, 137.7, 131.0, 128.6, 123.0, 122.7, 114.9, 63.7, 52.9, 14.9.

HRMS (EI-MS) for C₁₅H₁₅NO₃: calcd=257.1052, found=257.1050, Δm=0.8 ppm.

m.p.=102-104° C.

### Reference Compound NG-482 (Also Referred to as NG-384, NG-444 and TSch-42)

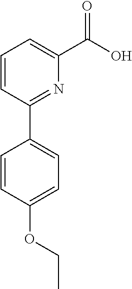 [SMILES: CCOc1ccc(-c2cccc(C(=O)O)n2)cc1]

The saponification of NG-442 was performed following the general procedure SA1.

Yield=470.8 mg slightly yellowish solid (1.935 mmol, 96%)

R_{f}=0.57 (CH₂Cl₂/MeOH=9+1+some drops HOAc; UV).

¹H-NMR (300 MHz, CDCl₃): δ=8.18-7.85 (m, 5H), 7.04 (d, J=8.8 Hz, 2H), 4.12 (q, J=7.0 Hz, 2H), 1.47 (t, J=7.0 Hz, 3H).

### Example 2: NG-385

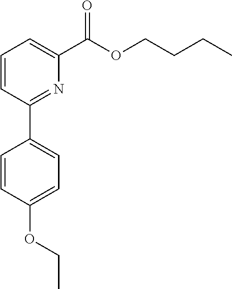 [SMILES: CCCCOC(=O)c1cccc(-c2ccc(OCC)cc2)n1]

The esterification of NG-384 (see NG-482) with 1-butanol was performed following the general procedure ES3.

Yield=81.8 mg colorless solid (0.273 mmol, 65%).

R_{f}=0.40 (cyclohexane/EtOAc=10+1; UV, KMnO₄).

¹H-NMR (300 MHz, CDCl₃): δ=8.14-7.89 (m, 3H), 7.83 (d, J=4.2 Hz, 2H), 6.99 (d, J=8.5 Hz, 2H), 4.42 (t, J=6.7 Hz, 2H), 4.09 (q, J=6.9 Hz, 2H), 1.94-1.69 (m, 2H), 1.60-1.35 (m, 5H), 1.00 (t, J=7.3 Hz, 3H). Minor grease and solvent impurities.

¹³C-NMR,APT (76 MHz, CDCl₃): δ=165.7, 160.4, 157.4, 148.3, 137.7, 131.0, 128.6, 122.7, 122.6, 114.8, 65.7, 63.7, 30.8, 19.4, 14.9, 13.9.

HRMS (EI-MS) for C₁₈H₂₁NO₃: calcd=299.1521, found=299.1521.

m.p.=78-81° C.

### Example 3: NG-386

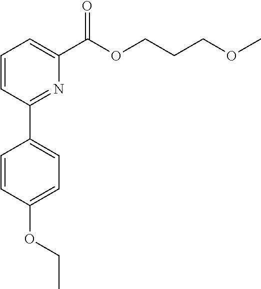 [SMILES: CCOc1ccc(-c2cccc(C(=O)OCCCOC)n2)cc1]

The esterification of NG-384 (see NG-482) with 3-methoxy-1-propanol was performed following the general procedure ES3.

Yield=85.0 mg slightly yellow solid (0.270 mmol, 64%).

R_{f}=0.09 (cyclohexane/EtOAc=10+1; UV, KMnO₄).

¹H-NMR (300 MHz, CDCl₃): δ=8.08-7.91 (m, 3H), 7.84 (d, J=4.2 Hz, 2H), 6.99 (d, J=8.7 Hz, 2H), 4.51 (t, J=6.5 Hz, 2H), 4.09 (q, J=6.9 Hz, 2H), 3.57 (t, J=6.2 Hz, 2H), 3.37 (s, 3H), 2.11 (p, J=6.3 Hz, 2H), 1.44 (t, J=6.9 Hz, 3H).

¹³C-NMR,APT (76 MHz, CDCl₃): δ=165.6, 160.4, 157.4, 148.1, 137.7, 130.9, 128.6, 122.8, 122.7, 114.8, 69.4, 63.7, 63.1, 58.9, 29.2, 14.9.

HRMS (EI-MS) for C₁₈H₂₁NO₄: calcd=315.1471, found=315.1468, Δm=1.0 ppm.

m.p.=36-38° C.

### Example 4: NG-387

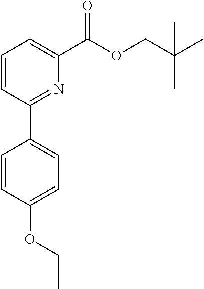 [SMILES: CCOc1ccc(-c2cccc(C(=O)OCC(C)(C)C)n2)cc1]

The esterification of NG-384 (see NG-482) with 2,2-dimethyl-1-propanol was performed following the general procedure ES1 with the modification that 1.4 eq of the alcohol were used Yield=85.4 mg yellow solid (0.272 mmol, 73%).

R_{f}=0.18 (cyclohexane/EtOAc=15+1; KMnO₄).

¹H-NMR (300 MHz, CDCl₃): δ=8.06 (d, J=8.7 Hz, 2H), 8.00-7.91 (m, 1H), 7.89-7.79 (m, 2H), 6.99 (d, J=8.7 Hz, 2H), 4.24-4.02 (m, 4H), 1.44 (t, J=7.0 Hz, 3H), 1.08 (s, 9H). Grease impurities.

¹³C-NMR,APT (76 MHz, CDCl₃): δ=165.5, 160.4, 157.3, 148.3, 137.6, 131.0, 128.6, 122.5, 114.8, 74.9, 63.7, 31.9, 26.7, 14.9. 1 carbon signal is missing maybe due to overlap.

HRMS (EI-MS) for C₁₉H₂₃NO₃: calcd=313.1678, found=313.1671, Δm=2.2 ppm.

m.p.=89-92° C.

### Intermediate NG-388

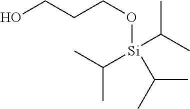

The synthesis is based on the literature (Angew. Chem. Int. Ed. 2011, 50, 3730-3733).

A Schienk tube was dried under vacuum and was charged with 320 mg (8.00 mmol) of a 60 m % NaH dispersion in mineral oil and 15 mL anhydrous THF. Subsequently, 475 μL (6.57 mmol) of 1,3-propanediol were added over the course of 10 min and the mixture is further stirred for 30 min at rt, after which time 1.27 g (6.57 mmol) TIPS-Cl were added. The mixture stirred at rt overnight, after which time 20 mL H₂O were added and the aqueous layer was extracted with EtOAc (3×25 mL). The combined organic layers were washed with brine (1×20 mL), dried over MgSO₄, filtered and the solvent was removed under reduced pressure and 1.57 g (<6.75 mmol, <100%) of NG-388 were isolated as colorless oil (technical purity).

¹H-NMR (300 MHz, CDCl₃): δ=3.93 (t, J=5.5 Hz, 2H), 3.83 (t, J=5.3 Hz, 2H), 2.27 (s, 1.6H), 1.91-1.73 (m, 2H), 1.18-0.95 (m, 28H—should be 21H).

### Example 5: NG-390

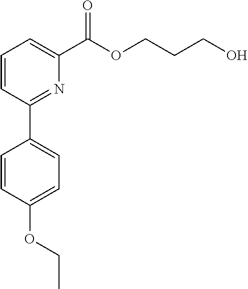 [SMILES: CCOc1ccc(-c2cccc(C(=O)OCCCO)n2)cc1]

A Schlenk tube was dried under vacuum and charged with 101.4 mg (417 μmol) NG-384 (see NG-482), 2 mL anhydrous THF, and 89.8 mg (468 μmol) EDC*HCl. Subsequently, 167.7 mg (721 μmol) NG-388 and 4.6 mg (37.7 μmol) DMAP were added at 0° C. (ice-bath) and the mixture was stirred at rt overnight. Subsequently, the mixture was filtered and the solvent was removed under reduced pressure and purification via column chromatography yielded a colorless oil that was dissolved in 20 mL THF. Subsequently, TBAF*3H₂O were added and the colorless solution was stirred at rt overnight, after which time the solvent was removed under reduced pressure. The crude product was purified via two consecutive column chromatographies (cyclohexane/EtOAc=1+1; cyclohexane/EtOAc=3+2) and 33 mg (0.1095 mmol, 26% over two steps) of NG-390 were isolated as colorless solid.

R_{f}=0.18 (cyclohexane/EtOAc=3+2; KMnO₄).

¹H-NMR (300 MHz, CDCl₃): δ=7.91 (dd, J=41.2 Hz, 5.9 Hz, 5H), 6.99 (d, J=8.2 Hz, 2H), 4.57 (t, J=5.5 Hz, 2H), 4.09 (dd, J=13.5 Hz, 6.6 Hz, 2H), 3.93-3.74 (m, 2H), 2.83 (s, 1H), 2.17-1.96 (m, 2H), 1.43 (t, J=6.8 Hz, 3H). Minor grease impurities.

¹³C-NMR,APT (76 MHz, CDCl₃): δ=165.7, 160.5, 157.5, 147.7, 137.8, 130.7, 128.6, 123.2, 122.7, 114.9, 64.2, 63.7, 60.6, 31.7, 14.9.

HRMS (EI-MS) for C₁₇H₁₉NO₄: calcd=301.1314, found=301.11313, Δm=0.3 ppm.

m.p.=61-63° C.

### Example 6: NG-399

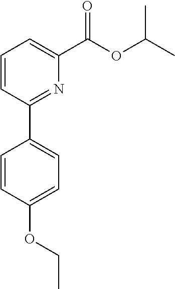 [SMILES: CCOc1ccc(-c2cccc(C(=O)OC(C)C)n2)cc1]

The esterification of NG-384 (see NG-482) with 2-propanol was performed following the general procedure ES1.

Yield=21.3 mg slightly yellow solid (0.0747 mmol, 52%).

R_{f}=0.21 (cyclohexane/EtOAc=10+1; KMnO₄).

¹H-NMR (300 MHz, CDCl₃): δ=8.04 (d, J=8.6 Hz, 2H), 7.98-7.90 (m, 1H), 7.83 (d, J=4.2 Hz, 2H), 6.99 (d, J=8.6 Hz, 1H), 5.44-5.23 (m, 1H), 4.09 (q, J=6.9 Hz, 2H), 1.55-1.30 (m, 9H).

¹³C-NMR,APT (76 MHz, CDCl₃): δ=165.0, 160.4, 157.3, 148.5, 137.7, 130.9, 128.7, 122.7, 122.6, 114.8, 69.5, 63.7, 22.0, 14.9.

HRMS (EI-MS) for C₁₇H₁₉NO₃: calcd=285.1365, found=285.1361, Δm=1.4 ppm.

m.p.=96-98° C.

### Example 7: NG-400

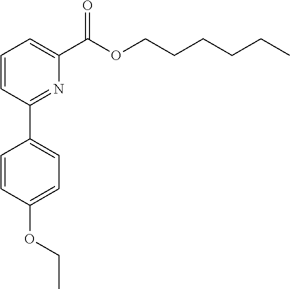 [SMILES: CCCCCCOC(=O)c1cccc(-c2ccc(OCC)cc2)n1]

The esterification of NG-384 (see NG-482) with 1-hexanol was performed following the general procedure ES1 with the modification that 0.1 eq DMAP were used. An additional column chromatography was performed for purification.

Yield=26.0 mg colorless solid (0.0794 mmol, 54%).

R_{f}=0.19 (cyclohexane/EtOAc=15+1; KMnO₄).

¹H NMR (300 MHz, CDCl₃) δ 8.17-7.76 (m, 5H), 6.98 (d, J=8.7 Hz, 2H), 4.40 (t, J=6.8 Hz, 2H), 4.09 (q, J=6.9 Hz, 2H), 1.92-1.72 (m, 2H), 1.55-1.28 (m, 9H), 1.00-0.82 (m, 3H).

¹³C-NMR,APT (76 MHz, CDCl₃): δ 165.7, 160.4, 157.4, 148.3, 137.6, 131.0, 128.6, 122.6 (2×), 114.8, 66.0, 63.7, 31.6, 28.8, 25.8, 22.7, 14.9, 14.1.

HRMS (EI-MS) for C₂₀H₂₅NO₃: calcd=327.1834, found=327.1821, Δm=4.0 ppm.

m.p.=48-51° C.

### Example 8: NG-402

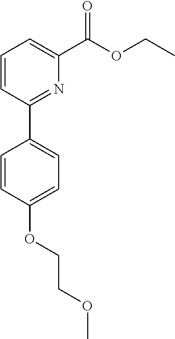 [SMILES: CCOC(=O)c1cccc(-c2ccc(OCCOC)cc2)n1]

The coupling of ethyl-6-bromopicolinate with 4-(2-methoxyethoxy)benzeneboronic acid was performed following the general procedure SC3 with the modification that 2.33 eq CsF were used.

Yield=96.9 mg colorless solid (0.322 mmol, 63%).

R_{f}=0.12 (cyclohexane/EtOAc=5+1; KMnO₄).

¹H-NMR (300 MHz, CDCl₃): δ=8.14-7.92 (m, 3H), 7.83 (d, J=4.6 Hz, 2H), 7.02 (d, J=8.7 Hz, 2H), 4.48 (q, J=7.1 Hz, 2H), 4.28-4.09 (m, 2H), 3.85-3.69 (m, 2H), 3.45 (s, 3H), 1.45 (t, J=7.1 Hz, 3H).

¹³C-NMR,APT (76 MHz, CDCl₃): δ=165.7, 160.2, 157.3, 148.3, 137.67, 131.4. 128.6, 122.8, 122.7, 115.0, 71.1, 67.5, 61.9, 59.4, 14.4.

HRMS (EI-MS) for C₁₇H₁₉NO₄: calcd=301.11314, found=301.1329, Δm=5.0 ppm.

m.p.=89-94° C.

### Reference Compound NG-403

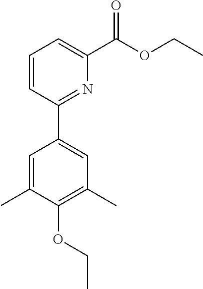 [SMILES: CCOC(=O)c1cccc(-c2cc(C)c(OCC)c(C)c2)n1]

The coupling of ethyl-6-bromopicoinate with 4-ethoxy-3,5-dimethylphenylboronic acid was performed following the general procedure SC3.

Yield=118.8 mg slightly yellow oil (0.397 mmol, 82%).

R_{f}=0.20 (cyclohexane/EtOAc=10+1; KMnO₄).

¹H-NMR (300 MHz, CDCl₃): δ=8.03-7.95 (m, 1H), 7.92-7.78 (m, 1H), 7.70 (s, 1H), 4.49 (q, J=7.1 Hz, 2H), 3.88 (q, J=7.0 Hz, 2H), 2.36 (s, 6H), 1.45 (2×t, J=7.0 Hz, 7.3 Hz, 6H).

¹³C-NMR,APT (76 MHz, CDCl₃): δ=165.5, 157.8, 148.2, 137.7, 133.7, 131.6, 127.9, 123.5, 122.9, 68.1, 62.0, 16.6, 15.9, 14.4. 1 carbon signal is missing maybe due to overlap.

HRMS (EI-MS) for C₁₈H₂₁NO₃: calcd=299.1521, found=299.1535, Δm=4.7 ppm.

### Reference Compound NG-408

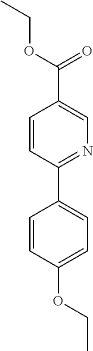 [SMILES: CCOC(=O)c1ccc(-c2ccc(OCC)cc2)nc1]

The coupling of ethyl 2-bromo-5-pyridinecarboxylate with 4-ethoxyphenylboronic acid was performed following the general procedure SC3.

Yield=69.9 mg colorless solid (0.258 mmol, 57%).

R_{f}=0.23 (cyclohexane/EtOAc=10+1; UV, KMnO₄).

¹H-NMR (300 MHz, CDCl₃): δ=9.23 (d, J=1.3 Hz, 1H), 8.30 (dd, J=8.3 Hz, 2.0 Hz, 1H), 8.02 (d, J=8.7 Hz, 2H), 7.73 (d, J=8.3 Hz, 1H), 7.00 (d, J=8.7 Hz, 2H), 4.42 (q, J=7.1 Hz, 2H), 4.10 (q, J=6.9 Hz, 2H), 1.55-1.25 (m, 6H).

¹³C-NMR,APT (76 MHz, CDCl₃): δ=165.6, 160.9, 160.5, 150.9, 137.9, 130.7, 128.9, 123.9, 119.0, 115.0, 63.8, 61.4, 14.9, 14.4.

HRMS (EI-MS) for C₁₆H₁₇NO₃: calcd=271.1208, found=271.1206, Δm=0.7 ppm.

m.p.=106-108° C.

### Example 9: NG-409

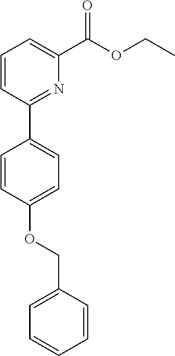 [SMILES: CCOC(=O)c1cccc(-c2ccc(OCc3ccccc3)cc2)n1]

The coupling of ethyl-6-bromopicolinate with 4-(benzyloxy)phenylboronic acid was performed following the general procedure SC1 with the modification that 0.96 eq boronic acid and 1.99 eq CsF were used, degassing was omitted, and that after stirring overnight at 80° C. (oil-bath) additional 0.45 eq boronic acid were added. The mixture was stirred another 3 d at 80° C.

Yield=134.5 mg yellow solid (0.403 mmol, 79%).

R_{f}=0.31 (cyclohexane/EtOAc=5+1; UV, KMnO₄).

¹H-NMR (300 MHz, CDCl₃): δ=8.15-7.29 (m, 10H), 7.08 (d, J=8.7 Hz, 2H), 5.14 (s, 2H), 4.48 (q, J=7.1 Hz, 2H), 1.46 (t, J=7.1 Hz, 3H).

¹³C-NMR,APT (76 MHz, CDCl₃): δ=165.7, 160.2, 157.4, 148.4, 137.6, 136.9, 131.5, 128.8, 128.7, 128.2, 127.6, 122.8, 122.7, 115.3, 70.2, 61.9, 14.5.

HRMS (EI-MS) for C₂₁H₁₉NO₃: calcd=333.1365, found=333.1374, Δm=2.7 ppm.

m.p.=143-145° C.

### Intermediate NG-412

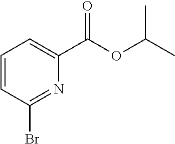

The compound is known in the literature (Synth. Commun. 2014, 44, 2121-2127). The esterification of 6-bromopyridine-2-carboxylic acid with 2-propanol was performed following the general procedure ES1 with the modification that 0.1 eq DMAP and 50 mL anhydrous THF were used for 1.49 g 6-bromopyridine-2-carboxylic acid.

¹H-NMR (300 MHz, CDCl₃): δ=8.03 (dd, J=6.7 Hz, 1.5 Hz, 1H), 7.82-7.59 (m, 2H), 5.37-5.17 (m, 1H), 1.40 (d, J=6.3 Hz, 6H).

¹³C-NMR,APT (76 MHz, CDCl₃): δ=163.4, 149.6, 142.4, 139.1, 131.7, 124.0, 70.2, 21.9.

Yield=840 mg colorless solid (3.44 mmol, 47%).

R_{f}=0.24 (cyclohexane/EtOAc=10+1; KMnO₄).

m.p.=78-80° C.

### Example 10: NG-415

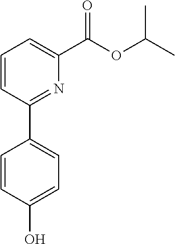 [SMILES: CC(C)OC(=O)c1cccc(-c2ccc(O)cc2)n1]

The coupling of NG-412 (isopropyl 6-bromopicolinate) with 4-hydroxyphenylboronic acid was performed following the general procedure SC2 with the modification that 1.0 eq boronic acid were used.

Yield=294.9 mg yellow solid (1.15 mmol, 56%).

R_{f}=0.26 (cyclohexane/EtOAc=3+1; UV, KMnO₄).

¹H-NMR (300 MHz, CDCl₃): δ=8.12-7.68 (m, 5H), 6.96 (d, J=8.2 Hz, 2H), 5.45-5.25 (m, 1H), 1.42 (d, J=6.1 Hz, 6H).

¹³C-NMR,APT (76 MHz, CDCl₃): δ=165.4, 157.9, 148.1, 137.8, 130.6, 128.9, 123.2, 122.6, 116.1, 69.9, 22.0.

HRMS (EI-MS) for C₁₅H₁₅NO₃: calcd=257.1052, found=257.1062, Δm=3.9 ppm.

m.p.=154-157° C.

### Example 11: NG-416

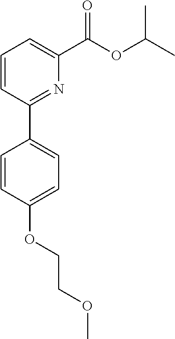 [SMILES: COCCOc1ccc(-c2cccc(C(=O)OC(C)C)n2)cc1]

The coupling of NG-412 (isopropyl 6-bromopicolinate) with [4-(2-methoxyethoxy)phenyl]boronic acid was performed following the general procedure SC3.

Yield=121.8 mg yellowish solid (0.386 mmol, 87%).

R_{f}=0.27 (cyclohexane/EtOAc=3+1; UV, KMnO₄).

¹H-NMR (300 MHz, CDCl₃): δ=8.19-7.71 (m, 5H), 7.02 (d, J=8.7 Hz, 2H), 5.43-5.21 (m, 1H), 4.28-4.05 (m, 2H), 3.88-3.67 (m, 2H), 3.47 (s, 3H), 1.43 (d, J=6.2 Hz, 6H). Minor solvent impurities.

¹³C-NMR,APT (76 MHz, CDCl₃): δ=165.1, 160.2, 157.3, 148.7, 137.6, 131.5, 128.6, 122.6 (2×), 115.0, 71.1, 69.5, 67.5, 59.4, 22.0.

HRMS (EI-MS) for C₁₈H₂₁NO₄: calcd=315.1471, found=315.1490, Δm=6.0 ppm.

m.p.=75-77° C.

### Example 12: NG-417

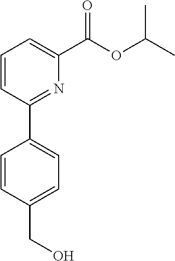 [SMILES: CC(C)OC(=O)c1cccc(-c2ccc(CO)cc2)n1]

The coupling of NG-412 (isopropyl 6-bromopicolinate) with 4-(hydroxymethyl)phenylboronic acid was performed following the general procedure SC1 with the modification that 1.0 eq boronic acid were used and that the reaction mixture was stirred 4 times overnight.

Yield=63.7 mg slightly orange solid (0.235 mmol, 38%).

R_{f}=0.18 (cyclohexane/EtOAc=2+1; UV, KMnO₄).

¹H-NMR (300 MHz, CDCl₃): δ=8.20-7.80 (m, 5H), 7.45 (d, J=6.6 Hz, 2H), 5.44-5.23 (m, 1H), 4.74 (s, 2H), 2.65 (s, 1H), 1.44 (d, J=6.1 Hz, 6H). Minor solvent impurities.

¹³C-NMR,APT (76 MHz, CDCl₃): δ=164.8, 157.3, 148.5, 142.6, 138.1, 137.5, 127.6, 127.4, 123.6, 123.4, 69.7, 65.0, 22.0.

HRMS (EI-MS) for C₁₆H₁₇NO₃: calcd=271.1208, found=271.1215, Δm=2.6 ppm.

m.p.=129-131° C.

### Example 13: NG-418

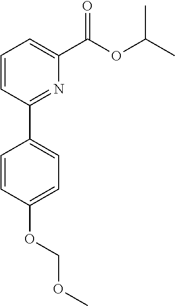 [SMILES: COCOc1ccc(-c2cccc(C(=O)OC(C)C)n2)cc1]

A Schlenk tube was dried under vacuum an was a with 80.0 mg (311 μmol) NG-415, 1 mL anhydrous DMF, and 15.6 mg (390 μmol) of a 60 m % NaH dispersion in mineral oil. The mixture was stirred for 15 min at rt and 30 μL 395 μmol) chloromethyl methyl ether were added. The mixture was stirred 45 min at rt and overnight at 100° C. (oil-bath), until which time TLC indicated all starting material to be consumed. The mixture was poured into 5 mL of a saturated aqueous NH₄Cl solution and the aqueous layer was extracted with EtOAc (3×5 mL). The combined organic layers were dried over Na₂SO₄, filtered and the solvent was removed under reduced pressure. The crude product was purified via column chromatography (cyclohexane/EtOAc=3+1) and 53.1 mg (0.176 mmol, 57%) of NG-418 were isolated as cloudy, colorless oil.

R_{f}=0.31 (cyclohexane/EtOAc=3+1; UV, KMnO₄).

¹H-NMR (300 MHz, CDCl₃): δ=8.16-7.90 (m, 3H), 7.83 (d, J=4.1 Hz, 2H), 7.14 (d, J=8.6 Hz, 2H), 5.40-5.27 (m, 1H), 5.23 (s, 2H), 3.50 (s, 3H), 1.43 (d, J=6.2 Hz, 6H).

¹³C-NMR,APT (76 MHz, CDCl₃): δ=165.0, 158.6, 157.2, 148.6, 137.7, 132.3, 128.7, 122.8 (2× according to HSQC), 116.5, 94.5, 69.5, 56.2, 22.0.

HRMS (EI-MS) for C₁₇H₁₉NO₄: calcd=301.1314, found=301.1319, Δm=1.7 ppm.

### Example 14: NG-423

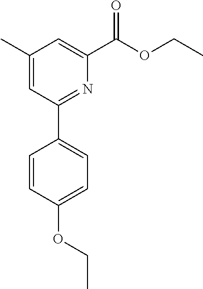 [SMILES: CCOC(=O)c1cc(C)cc(-c2ccc(OCC)cc2)n1]

The coupling of ethyl 6-chloro-4-methylpyridine-2-carboxylate with 4-ethoxyphenylboronic acid was performed following the general procedure SC1 with the modification that 2.2 eq CsF were used and that the reaction mixture was stirred 4 times overnight. An additional crystallization was performed for purification.

Yield=52.7 mg colorless crystals (0.185 mmol, 37%).

R_{f}=0.50 (cyclohexane/EtOAc=3+1; UV, KMnO₄).

¹H-NMR (300 MHz, CDCl₃): δ=8.00 (d, J=8.6 Hz, 2H), 7.82 (s, 1H), 7.65 (s, 1H), 6.98 (d, J=8.6 Hz, 2H), 4.47 (q, J=7.1 Hz, 2H), 4.09 (q, J=6.9 Hz, 2H), 2.46 (s, 3H), 1.45 (2×t, 6H).

¹³C-NMR,APT (76 MHz, CDCl₃): δ=165.8, 160.4, 157.3, 149.1, 148.0, 130.9, 128.7, 123.8, 123.8, 114.8, 63.7, 61.9, 21.4, 14.9, 14.5.

HRMS (EI-MS) for C₁₇H₁₉NO₃: calcd=285.1365, found=285.1375, Δm=3.9 ppm.

m.p.=90-91° C.

### Example 15: NG-427

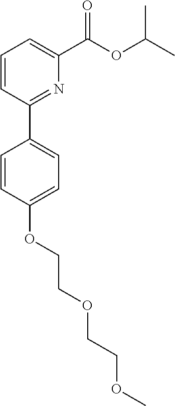 [SMILES: COCCOCCOc1ccc(-c2cccc(C(=O)OC(C)C)n2)cc1]

A Schienk tube was dried under vacuum and was charged with 60.7 mg (236 μmol) NG-415, 1 mL anhydrous DMF, and 14.5 mg (363 μmol) of a 60 m % NaH dispersion in mineral oil. The mixture was stirred for 15 min at rt and 50 μL (371 μmol) 2-(2-methoxyethoxy)ethyl bromide were added. The mixture was stirred 80 min at rt and overnight at 100° C. (oil-bath), until which time TLC indicated all starting material to be consumed. The mixture was poured into 5 mL of a saturated aqueous NH₄Cl solution and the aqueous layer was extracted with EtOAc (3×5 mL). The combined organic layers were dried over Na₂SO₄, filtered and the solvent was removed under reduced pressure. The crude product was purified via column chromatography (cyclohexane/EtOAc=2+1) and 47.2 mg (0.131 mmol, 56%) of NG-427 were isolated as colorless solid.

R_{f}=0.20 (cyclohexane/EtOAc=2+1; KMnO₄).

¹H-NMR (300 MHz, CDCl₃): δ=8.04 (d, J=8.6 Hz, 2H), 7.99-7.89 (m, 1H), 7.82 (d, J=4.1 Hz, 2H), 7.01 (d, J=8.6 Hz, 2H), 5.43-5.23 (m, 1H), 4.29-4.11 (m, 2H), 3.97-3.83 (m, 2H), 3.79-3.68 (m, 2H), 3.65-3.53 (m, 2H), 3.39 (s, 3H), 1.43 (d, J=6.2 Hz, 6H).

¹³C-NMR,APT (76 MHz, CDCl₃): δ=165.0, 160.2, 157.2, 148.6, 137.6, 131.3, 128.6, 122.6, 115.0, 72.1, 70.9, 69.9, 69.5, 67.6, 59.2, 22.0. 1 carbon signal is missing maybe due to overlap.

HRMS (EI-MS) for C₂₀H₂₅NO₅: calcd=359.1733, found=359.1738, Δm=1.4 ppm.

m.p.=45-46° C.

### Example 16: NG-428

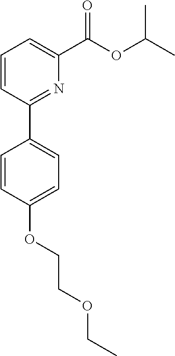 [SMILES: CCOCCOc1ccc(-c2cccc(C(=O)OC(C)C)n2)cc1]

A Schlenk tube was dried under vacuum and was charged with 64.5 mg (261 μmol) NG-415, 1 mL anhydrous DMF, and 17.1 mg (428 μmol) of a 60 m % NaH dispersion in mineral oil. The mixture was stirred for 15 min at rt and 50 μL (444 μmol) 2-bromoethyl ethyl ether were added. The mixture was stirred 80 min at rt and overnight at 100° C. (oil-bath), until which time TLC indicated all starting material to be consumed. The mixture was poured into 5 mL of a saturated aqueous NH₄Cl solution and the aqueous layer was extracted with EtOAc (3×5 mL). The combined organic layers were dried over Na₂SO₄, filtered and the solvent was removed under reduced pressure. The crude product was purified via column chromatography (cyclohexane/EtOAc=4+1) and 43.7 mg (0.133 mmol, 53%) of NG-428 were isolated as colorless solid.

R_{f}=0.26 (cyclohexane/EtOAc=4+1; KMnO₄).

¹H-NMR (300 MHz, CDCl₃): δ=8.04 (d, J=8.7 Hz, 2H), 7.99-7.89 (m, 1H), 7.83 (d, J=4.1 Hz, 2H), 7.02 (d, J=8.7 Hz, 2H), 5.44-5.22 (m, 1H), 4.32-4.09 (m, 2H), 3.90-3.73 (m, 2H), 3.62 (q, J=7.0 Hz, 2H), 1.43 (d, J=6.2 Hz, 6H), 1.26 (t, J=7.0 Hz, 3H).

¹³C-NMR,APT (76 MHz, CDCl₃): δ=165.1, 160.3, 157.3, 148.6, 137.6, 131.3, 128.6, 122.6, 115.0, 69.5, 69.0, 67.7, 67.0, 22.0, 15.3. 1 carbon signal missing maybe due to overlap.

HRMS (EI-MS) for C₁₉H₂₃NO₄: calcd=329.1627, found=329.1659, Δm=9.7 ppm.

m.p.=46-47° C.

### Example 17: NG-432

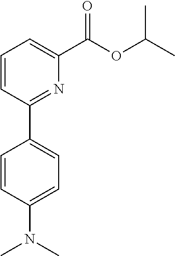 [SMILES: CC(C)OC(=O)c1cccc(-c2ccc(N(C)C)cc2)n1]

The coupling of isopropyl 6-bromopicolinate with 4-(dimethylamino)benzeneboronic acid was performed following the general procedure SC1 with the modification that 2.34 eq CsF were used.

Yield=26.5 mg colorless solid (0.0932 mmol, 32%).

R_{f}=0.31 (cyclohexane/EtOAc=5+1; KMnO₄).

¹H-NMR (300 MHz, CDCl₃): δ=8.15-7.68 (m, 5H), 6.84 (d, J=5.8 Hz, 2H), 5.40-5.22 (m, 1H), 3.03 (s, 6H), 1.43 (d, J=6.2 Hz, 6H).

¹³C-NMR (126 MHz, CDCl₃): δ=165.3, 157.6, 148.5, 137.3, 128.2, 121.9 (2×), 112.8, 69.3, 40.8, 22.1. 2 carbon signals are missing maybe due to overlap.

HRMS (EI-MS) for C₁₇H₂₀N₂O₂: calcd=284.1525, found=284.1526, Δm=0.4 ppm.

m.p.=134-140° C.

### Example 18: NG-433

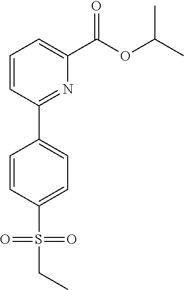 [SMILES: CCS(=O)(=O)c1ccc(-c2cccc(C(=O)OC(C)C)n2)cc1]

The coupling of isopropyl 6-bromopicolinate with 4-(ethanesulfonyl)benzeneboronic acid was performed following the general procedure SC1.

Yield=99.4 mg colorless solid (0.298 mmol, 72%).

R_{f}=0.42 (cyclohexane/EtOAc=1+1; UV, KMnO₄).

¹H-NMR (300 MHz, CDCl₃): δ=8.27 (d, J=8.2 Hz, 2H), 8.16-7.85 (m, 5H), 5.41-5.26 (m, 1H), 3.14 (q, J=7.3 Hz, 2H), 1.44 (d, J=6.2 Hz, 6H), 1.28 (t, J=7.4 Hz, 3H). Minor solvent impurities.

¹³C-NMR,APT (76 MHz, CDCl₃): δ=164.6, 155.6, 149.2, 143.7, 139.0, 138.2, 128.9, 128.2, 124.5, 124.0, 69.9, 50.8, 22.0, 7.6.

HRMS (EI-MS) for C₁₇H₁₉NO₄S: calcd=333.1035, found=333.1042, Δm=2.1 ppm.

m.p.=126-129° C.

### Example 19: NG-434

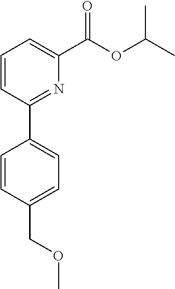 [SMILES: COCc1ccc(-c2cccc(C(=O)OC(C)C)n2)cc1]

The coupling of isopropyl 6-bromopicolinate with 4-(methoxymethyl)benzeneboronic acid was performed following the general procedure SC1.

Yield=93.1 mg colorless solid (0.326 mmol, 79%).

R_{f}=0.34 (cyclohexane/EtOAc=4+1; UV, KMnO₄).

¹H-NMR (300 MHz, CDCl₃): δ=8.19-7.78 (m, 5H), 7.45 (d, J=8.0 Hz, 2H), 5.32 (dd, J=12.4 Hz, 6.2 Hz, 1H), 4.52 (s, 2H), 3.40 (s, 3H), 1.43 (d, J=6.2 Hz, 6H).

¹³C-NMR,APT (76 MHz, CDCl₃): δ=165.0, 157.4, 148.8, 139.7, 138.0, 137.7, 128.2, 127.4, 123.3, 74.4, 69.5, 58.2, 22.0. 1 carbon signal is missing maybe due to overlap.

HRMS (EI-MS) for C₁₇H₁₉NO₃: calcd=285.135, found=285.1375, Δm=3.9 ppm.

m.p.=55-58° C.

### Intermediate NG-435

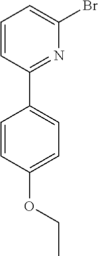 [SMILES: CCOc1ccc(-c2cccc(Br)n2)cc1]

This compound is known in the literature and was prepared analogously (Eur. J. Org. Chem. 2014, 2942-2955).

A Schlenk tube was dried under vacuum and was charged with 1.44 g (6.06 mmol) 2,6-dibromopyridine, 1.01 g (6.06 mmol) 4-ethoxyphenylboronic acid, 5.13 g (48.4 mmol) Na₂CO₃, 224.4 mg (3.01 mmol) KCl, 99.0 mg (0.377 mmol) PPh₃, and 211.7 mg (0.183 mmol) Pd(PPh₃)₄. Subsequently, a previously degassed solution of 40 mL toluene, 10 mL EtOH, and 20 mL H₂O were added and the mixture was stirred at rt for 5 d, after which time 50 mL H₂O were added and the aqueous layer was extracted with EtOAc (1×50 ml+2×30 mL). The combined organic layers were washed with H₂O (1×50 mL) and the aqueous layer was back-extracted with EtOAc (2×50 mL). All organic layers were combined, dried over Na₂SO₄, filtered, and the solvent was removed under reduced pressure. The crude product was purified via column chromatography (cyclohexane/EtOAc=3+1) and 1.39 g of impure NG-435 were isolated as yellow solid.

### Reference Compound NG-437

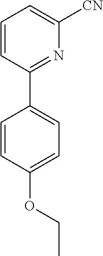 [SMILES: CCOc1ccc(-c2cccc(C#N)n2)cc1]

The procedure is based on the synthesis of a similar substrate (Bioorg. Med. Chem. 2004, 12, 5909-5915).

Caution: CuCN and NaCN are extremely toxic. Do not acidify as this would lead to the formation of toxic gaseous HCN!

A Schlenk tube was dried under vacuum and was charged with 302 mg (1.09 mmol) crude NG-435, 3 mL anhydrous DMF, 81.9 mg (0.914 mmol) CuCN, 49.4 mg (1.01 mmol) NaCN, and the mixture was stirred at 160° C. (oil-bath) overnight. To the mixture were added 8 mL H₂O and the aqueous layer was extracted with EtOAc (1×15 mL, 2×8 mL). The combined organic layers were dried over Na₂SO₄, filtered, and the solvent was removed under reduced pressure. The crude product was purified via column chromatography (cyclohexane/EtOAc=4+1) and 161 mg (0.719 mmol, 55% calc. over 2 steps) of NG-437 were isolated as colorless powder.

R_{f}=0.31 (cyclohexane/EtOAc=4+1; UV, KMnO₄).

¹H-NMR (300 MHz, CDCl₃): δ=8.13-7.72 (m, 4H), 7.54 (d, J=6.8 Hz, 1H), 6.99 (d, J=8.5 Hz, 2H), 4.10 (q, J=6.7 Hz, 2H), 1.45 (t, J=6.8 Hz, 3H).

¹³C-NMR,APT (76 MHz, CDCl₃): δ=160.9, 158.7, 137.6, 133.8, 129.7, 128.6, 125.9, 122.7, 117.7, 115.0, 63.8, 14.9.

HRMS (EI-MS) for C₁₄H₁₂N₂O: calcd=224.0950, found=224.0949, Δm=0.5 ppm.

m.p.=109-111° C.

### Example 20: NG-441

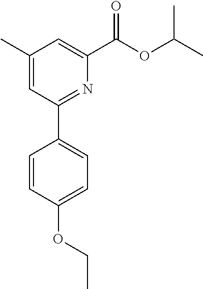 [SMILES: CCOc1ccc(-c2cc(C)cc(C(=O)OC(C)C)n2)cc1]

The saponification of NG-423 was performed following the general procedure SA1. The esterification with 2-propanol was performed following the general procedure ES2 with the modification that 0.27 eq DMAP were used.

Yield=15.0 mg colorless solid (0.0501 mmol, 47% calc. over 2 steps).

R_{f}=0.38 (cyclohexane/EtOAc=5+1; KMnO₄).

¹H-NMR (300 MHz, CDCl₃): δ=8.02 (d, J=8.7 Hz, 2H), 7.78 (s, 1H), 7.64 (s, 1H), 6.97 (d, J=8.7 Hz, 2H), 5.45-5.20 (m, 1H), 4.09 (q, J=6.9 Hz, 2H), 2.46 (s, 3H), 1.50-1.32 (m, 9H).

¹³C-NMR,APT (76 MHz, CDCl₃): δ=165.3, 160.3, 157.3, 148.8, 148.5, 131.2, 128.6, 123.7, 123.4, 114.7, 69.4, 63.7, 22.1, 21.4, 14.9.

HRMS (EI-MS) for C₁₈H₂₁NO₃: calcd=299.1521, found=299.1521.

m.p.=85-88° C.

### Example 21: NG-445

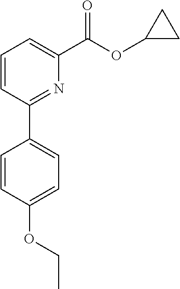 [SMILES: CCOc1ccc(-c2cccc(C(=O)OC3CC3)n2)cc1]

The esterification of NG-444 (see NG-482) with cyclopropanol was performed following the general procedure ES2.

Yield=28.5 mg colorless powder (0.101 mmol, 24%).

R_{f}=0.34 (cyclohexane/EtOAc=3+1; UV).

¹H-NMR (300 MHz, CDCl₃): δ=8.16-7.76 (m, 5H), 6.98 (d, J=8.6 Hz, 2H), 4.48-4.36 (m, 1H), 4.09 (q, J=6.9 Hz, 2H), 1.44 (t, J=6.9 Hz, 3H), 1.02-0.75 (m, 5H).

¹³C-NMR,APT (76 MHz, CDCl₃): δ=166.6, 160.4, 157.4, 147.9, 137.6, 131.0, 128.6, 122.8, 122.7, 114.8, 63.7, 50.3, 14.9, 5.5.

HRMS (EI-MS) for C₁₇H₁₇NO₃: calcd=283.1208, found=283.1218, Δm=3.5 ppm.

m.p.=114-115° C.

### Example 22: NG-447

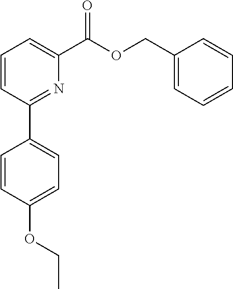 [SMILES: CCOc1ccc(-c2cccc(C(=O)OCc3ccccc3)n2)cc1]

A Schienk tube was dried under vacuum and was charged with 111.0 mg (0.456 mmol) NG-444 (see NG-482), 2 mL anhydrous DMSO, 94.5 mg (0.684 mmol) K₂CO₃, and 71 μL (0.598 mmol) benzyl bromide. The mixture was stirred at rt for 2 h, until which time TLC indicated all starting material to be consumed. Subsequently, 5 mL H₂O were added to the mixture and the aqueous layer was extracted with EtOAc (3×5 mL). The combined organic layers were dried over Na₂SO₄, filtered and the solvent was removed under reduced pressure.

The crude product was purified via column chromatography (cyclohexane/EtOAc=5+1) and 115.7 mg (0.347 mmol, 76%) of NG-447 were isolated as colorless solid.

R_{f}=0.33 (cyclohexane/EtOAc=5+1; UV, KMnO₄).

¹H-NMR (300 MHz, CDCl₃): δ 8.16-7.71 (m, 5H), 7.64-7.28 (m, 5H), 6.99 (d, J=8.7 Hz, 2H), 5.47 (s, 2H), 4.10 (q, J=6.9 Hz, 2H), 1.44 (t, J=6.9 Hz, 3H).

¹³C-NMR,APT (76 MHz, CDCl₃): δ 165.4, 160.4, 157.5, 148.0, 137.6, 136.1, 131.0, 128.7, 128.6, 128.4 (2×), 122.9, 122.8, 114.9, 67.4, 63.7, 14.9.

HRMS (EI-MS) for C₂₁H₁₉NO₃: calcd=333.1365, found=333.1377, Δm=3.6 ppm.

m.p.=80-81° C.

### Example 23: NG-451

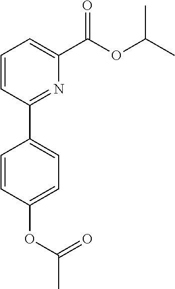 [SMILES: CC(=O)Oc1ccc(-c2cccc(C(=O)OC(C)C)n2)cc1]

A screw-cap vial was charged with 54.3 mg (0.211 mmol) NG-415, 2 mL CH₂Cl₂, 20 μL (0.253 mmol) pyridine, and 24 μL (0.253 mmol) acetic anhydride. The mixture was stirred at rt for 260 min, until which time TLC indicated all starting material to be consumed. Subsequently, 2 mL H₂ were added to the mixture and the organic layer was separated after extraction. The aqueous layer was extracted with CH₂Cl₂ (3×2 mL). The combined organic layers were dried over Na₂SO₄, filtered and the solvent was removed under reduced pressure. The crude product was purified via column chromatography (cyclohexane/EtOAc=4+1) and 43.8 mg (0.146 mmol, 69%) of NG-451 were isolated as slightly yellow solid.

R_{f}=0.29 (cyclohexane/EtOAc=4+1; UV, KMnO₄).

¹H-NMR (300 MHz, CDCl₃): δ=8.24-7.76 (m, 5H), 7.20 (d, J=8.3 Hz, 2H), 5.44-5.19 (m, 1H), 2.29 (s, 3H), 1.42 (d, J=6.2 Hz, 6H).

¹³C-NMR,APT (76 MHz, CDCl₃): δ=169.3, 164.9, 156.7, 151.9, 148.8, 137.7, 136.3, 128.5, 123.3, 123.1, 122.0, 69.5, 22.0, 21.2.

HRMS (EI-MS) for C₁₇H₁₇NO₄: calcd=299.1158, found=299.1163, Δm=1.7 ppm.

m.p.=71-75° C.

### Example 24: NG-460

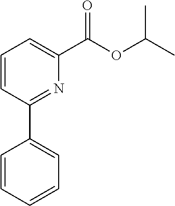 [SMILES: CC(C)OC(=O)c1cccc(-c2ccccc2)n1]

The coupling of isopropyl 6-bromopicolinate with phenylboronic acid was performed following the general procedure SC1 with the modification that following stirring at 80° C. (oil-bath) overnight, additional 1.45 eq boronic acid and 4.9 mol % PdCl₂(dppf) were added and the mixture was stirred overnight at 80° C.

Yield=65.0 mg slightly yellow, cloudy oil (0.269 mmol, 63%).

R_{f}=0.54 (cyclohexane/EtOAc=4+1; UV, KMnO₄).

¹H-NMR (300 MHz, CDCl₃): δ=8.16-7.83 (m, 5H), 7.62-7.34 (m, 4H), 5.42-5.25 (m, 1H), 1.44 (d, J=6.2 Hz, 6H).

¹³C-NMR (76 MHz, CDCl₃): δ=165.0, 157.6, 148.8, 138.6, 137.8, 129.6, 128.9, 127.4, 123.4, 123.3, 69.6, 22.0.

HRMS (EI-MS) for C₁₅H₁₅NO₂: calcd=241.1103, found=241.1104, Δm=0.4 ppm.

### Reference Compound NG-461

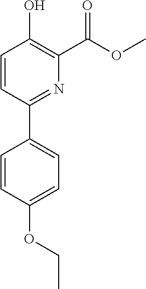 [SMILES: CCOc1ccc(-c2ccc(O)c(C(=O)OC)n2)cc1]

The coupling of methyl 6-bromo-3-hydroxypicolinate with 4-ethoxyphenylboronic acid was performed following the general procedure SC1.

Yield=65.2 mg colorless solid (0.239 mmol, 54%).

R_{f}=0.39 (cyclohexane/EtOAc=5+1; UV, KMnO₄).

¹H-NMR (300 MHz, CDCl₃): δ=7.84 (dd, J=19.0 Hz, 8.8 Hz, 3H), 7.44 (d, J=8.8 Hz, 1H), 6.97 (d, J=8.7 Hz, 2H), 4.19-3.95 (m, 6H), 1.44 (t, J=7.0 Hz, 3H).

¹³C-NMR,APT (76 MHz, CDCl₃): δ=170.1, 159.9, 157.6, 149.5, 130.7, 129.0, 128.1, 127.5, 126.6, 114.9, 63.7, 53.2, 14.9.

HRMS (EI-MS) for C₁₅H₁₅NO₄: calcd=273.1001, found=273.1010, Δm=3.3 ppm.

m.p.=114-116° C.

### Reference Compound NG-462

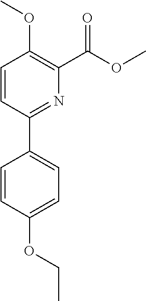 [SMILES: CCOc1ccc(-c2ccc(OC)c(C(=O)OC)n2)cc1]

The coupling of methyl 6-bromo-4-methoxypicolinate with 4-ethoxyphenylboronic acid was performed following the general procedure SC1 with the modification that 1.37 eq boronic acid and 2.62 eq CsF were used.

Yield=96.2 mg colorless solid (0.335 mmol, 80%).

R_{f}=0.22 (cyclohexane/EtOAc=3+1; UV, KMnO₄).

¹H NMR (300 MHz, CDCl₃) δ 7.88 (d, J=8.6 Hz, 2H), 7.74 (d, J=8.8 Hz, 1H), 7.38 (d, J=8.8 Hz, 1H), 7.38 (d, J=8.8 Hz, 1H), 6.95 (d, J=8.6 Hz, 2H), 4.15-3.85 (m, 8H), 1.43 (t, J=6.9 Hz, 3H).

¹³C-NMR,APT (76 MHz, CDCl₃): δ=165.9, 159.9, 153.7, 149.2, 138.9, 130.7, 128.2, 123.1, 121.2, 114.9, 63.8, 56.5, 52.8, 15.0.

HRMS (EI-MS) for C₁₆H₁₇NO₄: calcd=287.1158, found=287.1159, Δm=0.3 ppm.

m.p.=114-116° C.

### Intermediate NG-465

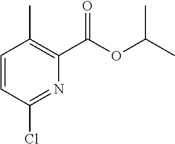

The esterification of 6-chloro-3-methylpicolinic acid with 2-propanol was performed following the general procedure ES2.

Yield=333.8 mg slightly yellow solid (1.56 mmol, 53%).

R_{f}=0.27 (cyclohexane/EtOAc=10+1; KMnO₄).

¹H-NMR (300 MHz, CDCl₃): δ=7.53 (d, J=8.1 Hz, 1H), 7.32 (d, J=8.1 Hz, 1H), 5.38-5.18 (m, 1H), 2.48 (s, 3H), 1.40 (d, J=6.3 Hz, 6H).

¹³C-NMR,APT (76 MHz, CDCl₃): δ=165.1, 148.9, 148.5, 142.3, 132.7, 126.3, 70.0, 21.9, 19.0.

HRMS (EI-MS) for C₁₀H₁₂ClNO₂: calcd=213.0557, found=213.0556, Δm=0.5 ppm.

m.p.=31-33° C.

### Example 25: NG-466

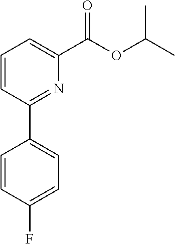 [SMILES: CC(C)OC(=O)c1cccc(-c2ccc(F)cc2)n1]

The coupling of isopropyl 6-bromopicolinate with 4-fluorophenylboronic acid was performed following the general procedure SC1.

Yield=103.1 mg cloudy, colorless oil (0.398 mmol, 97%).

R_{f}=0.40 (cyclohexane/EtOAc=5+1; UV).

¹H-NMR (300 MHz, CDCl₃): δ=8.19-7.77 (m, 5H), 7.16 (t, J=8.6 Hz, 2H), 5.42-5.23 (m, 1H), 1.44 (d, J=6.2 Hz, 6H).

¹³C-NMR,APT (76 MHz, CDCl₃): δ=165.6, 164.9, 162.3, 156.6, 148.8, 137.8, 134.8, 129.3, 129.2, 123.2, 123.0, 116.0, 115.7, 69.6, 22.0.

HRMS (EI-MS) for C₁₅H₁₄FNO₂: calcd=259.1009, found=259.1005, Δm=1.5 ppm.

### Reference Compound NG-469

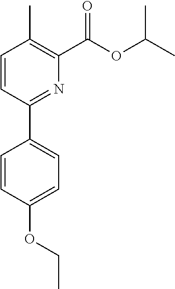 [SMILES: CCOc1ccc(-c2ccc(C)c(C(=O)OC(C)C)n2)cc1]

The coupling of NG-465 with 4-ethoxyphenylboronic acid was performed following the general procedure SC1 with the modification that after stirring overnight at 80° C. (oil-bath), additional 0.52 eq boronic acid and 1 mol % PdCl₂(dppf) were added and the mixture was stirred at 80° C. for additional 66 h.

Yield=93.7 mg clear, slightly yellowish oil (0.313 mmol, 63%).

R_{f}=0.50 (cyclohexane/EtOAc=3+1; UV, KMnO₄).

¹H-NMR (300 MHz, CDCl₃): δ=8.00 (d, J=8.7 Hz, 2H), 7.67 (q, J=8.2 Hz, 2H), 6.98 (d, J=8.6 Hz, 2H), 5.45-5.24 (m, 1H), 4.09 (q, J=6.9 Hz, 2H), 2.50 (s, 3H), 1.52-1.31 (m, 9H).

¹³C-NMR,APT (76 MHz, CDCl₃): δ=165.9, 160.5, 154.1, 148.2, 141.1, 131.1, 129.8, 128.7, 121.8, 114.9, 69.8, 63.7, 22.1, 18.9, 14.9.

HRMS (EI-MS) for C₁₈H₂₁NO₃: calcd=299.1521, found=299.1518, Δm=1.0 ppm.

### Example 26: NG-470

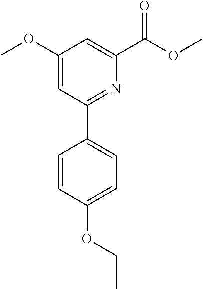

The coupling of methyl 6-chloro-4-methoxypicolinate with 4-ethoxyphenylboronic acid was performed following the general procedure SC1.

Yield=91.0 mg colorless solid (0.317 mmol, 64%).

R_{f}=0.36 (cyclohexane/EtOAc=3+1; UV, KMnO₄).

¹H-NMR (300 MHz, CDCl₃): δ=7.96 (d, J=8.7 Hz, 2H), 7.56 (d, J=2.1 Hz, 1H), 7.31 (d, J=2.1 Hz, 1H), 6.97 (d, J=8.7 Hz, 2H), 4.16-3.86 (m, 8H), 1.44 (t, J=7.0 Hz, 3H).

¹³C-NMR,APT (76 MHz, CDCl₃): δ=167.6, 165.9, 160.6, 159.0, 149.3, 130.6, 128.9, 114.8, 109.2 (2×), 63.7, 56.0, 53.1, 14.9.

HRMS (EI-MS) for C₁₆H₁₇NO₄: calcd=287.1158, found=287.1162, Δm=1.4 ppm.

m.p.=94-103° C.

### Intermediate NG-473

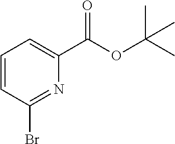

This compound is known in the literature known and was prepared analogously (By Collantes, Elizabeth Martha and Schwarz, Jacob Bradley From U.S. Pat. Appl. Publ., 20090197859, 6 Aug. 2009).

A round-bottom flask was charged with 1.098 g (5.44 mmol) 6-bromopyridine-2-carboxylic acid, 27.5 mL t-BuOH, and 3.8 mL pyridine. Subsequently, 2.11 g (11.1 mmol) TsCl were added at 0° C. (ice-bath) and the mixture was stirred overnight at rt, until which time TLC indicated all starting material to be consumed. To the mixture were poured 40 mL of a saturated aqueous NaHCO₃ solution and the mixture was stirred for 30 min at rt, after which time ˜½ solvent was removed under reduced pressure and the mixture was filtered. The filter residue was washed with H₂O, dried at 60° C. (oil-bath) under oil-pump vacuum, and 1.19 g (4.61 mmol, 85%) of NG-473 were isolated as colorless powder.

R_{f}=0.55 (CH₂Cl₂/MeOH=9+2+drops HOAc; UV).

¹H NMR (300 MHz, CDCl₃) δ 7.97 (dd, J=6.7 Hz, 1.3 Hz, 1H), 7.70-7.51 (m, 2H), 1.61 (s, 9H).

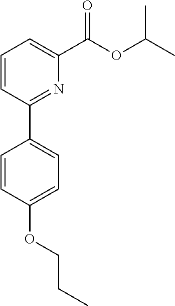 [SMILES: CCCOc1ccc(-c2cccc(C(=O)OC(C)C)n2)cc1]

### Example 27: NG-474

The coupling of isopropyl 6-bromopicolinate with 4-propoxyphenylboronic acid was performed following the general procedure SC1 with the modification that 8 mol % PdCl₂(dppf) were used.

Yield=93.0 mg colorless solid (0.311 mmol, 70%).

R_{f}=0.43 (cyclohexane/EtOAc=5+1; UV, KMnO₄)

¹H NMR (300 MHz, CDCl₃) δ 8.15-7.75 (m, 5H), 7.00 (d, J=8.7 Hz, 2H), 5.42-5.22 (m, 1H), 3.98 (t, J=6.6 Hz, 2H), 1.84 (dd, J=14.0 Hz, 7.0 Hz, 2H), 1.43 (t, J=7.7 Hz, 6H), 1.06 (t, J=7.4 Hz, 3H).

¹³C-NMR,APT (76 MHz, CDCl₃): δ=164.7, 160.8, 157.2, 148.3, 138.0, 130.4, 128.8, 122.9, 122.6, 114.9, 69.8, 69.7, 22.7, 22.0, 10.6.

HRMS (EI-MS) for C₁₈H₂₁NO₃: calcd=299.1521, found=299.1526, Δm=1.7 ppm.

m.p.=90-93° C.

### Reference Compound NG-477

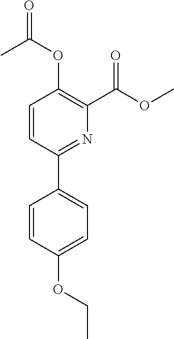 [SMILES: CCOc1ccc(-c2ccc(OC(C)=O)c(C(=O)OC)n2)cc1]

A screw-cap vial was charged with 46.1 mg (0.169 mmol) NG-461, 1 mL CH₂Cl₂, 16 μL (0.158 mmol) pyridine, and 19 μL (0.201 mmol) acetic anhydride and the mixture was stirred at rt. The mixture was stirred at rt for 100 min and additional 5 μL (0.0493 mmol) pyridine were added. After additional 95 min of stirring at rt, additional 10 μL (0.0986 mmol) pyridine and 10 μL (0.106 mmol) acetic acid were added. The mixture was stirred overnight, 2 mL H₂O were added to the mixture and the organic layer was separated after extraction. The aqueous layer was extracted with CH₂Cl₂ (2×2 mL). The combined organic layers were dried over MgSO₄, filtered and the solvent was removed under reduced pressure. The crude product was purified via column chromatography (cyclohexane/EtOAc=3+1) and 37.2 mg (0.118 mmol, 70%) of NG-477 were isolated as colorless solid.

R_{f}=0.28 (cyclohexane/EtOAc=3+1; UV, KMnO₄)

¹H NMR (300 MHz, CDCl₃) δ 7.94 (d, J=8.1 Hz, 2H), 7.84 (d, J=8.6 Hz, 1H), 7.53 (d, J=8.5 Hz, 1H), 6.98 (d, J=8.6 Hz, 2H), 4.09 (q, J=6.9 Hz, 2H), 3.97 (s, 3H), 2.38 (s, 3H), 1.44 (t, J=6.9 Hz, 3H).

¹³C-NMR,APT (76 MHz, CDCl₃): δ=169.4, 164.5, 160.4, 154.8, 146.1, 140.6, 133.1, 130.3, 128.7, 123.7, 114.9, 63.7, 52.9, 21.0, 14.9.

HRMS (EI-MS) for C₁₇H₁₇NO₅: calcd=315.1107, found=315.1120, Δm=4.1 ppm.

m.p.=120-123° C.

### Example 28: NG-480

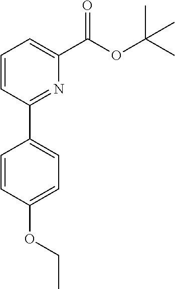 [SMILES: CCOc1ccc(-c2cccc(C(=O)OC(C)(C)C)n2)cc1]

The coupling of NG-473 with 4-ethoxyphenylboronic acid was performed following the general procedure SC1.

Yield=89.1 mg colorless solid (0.298 mmol, 75%).

R_{f}=0.33 (cyclohexane/EtOAc=7+1; UV).

¹H-NMR (300 MHz, CDCl₃): δ=8.20-7.76 (m, 5H), 6.98 (d, J=8.6 Hz, 2H), 4.09 (q, J=6.9 Hz, 2H), 1.64 (s, 9H), 1.44 (t, J=6.9 Hz, 3H).

¹³C-NMR,APT (76 MHz, CDCl₃): δ=164.2, 160.5, 157.1, 149.0, 137.8, 130.7, 128.7, 122.5 (2×), 114.8, 82.3, 63.7, 28.3, 14.9.

HRMS (EI-MS) for C₁₈H₂₁NO₃: calcd=299.1521, found=299.1532, Δm=3.7 ppm.

m.p.=69-73° C.

### Reference Compound NG-481

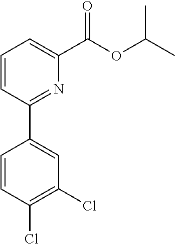 [SMILES: CC(C)OC(=O)c1cccc(-c2ccc(Cl)c(Cl)c2)n1]

The coupling of isopropyl 6-bromopicolinate was performed following the general procedure SC1.

Yield=115.0 mg colorless solid (0.371 mmol, 88%).

R_{f}=0.30 (cyclohexane/EtOAc=7+1; UV).

¹H-NMR (300 MHz, CDCl₃): δ=8.20 (d, J=1.8 Hz, 1H), 8.11-7.79 (m, 4H), 7.55 (d, J=8.4 Hz, 1H), 5.34 (dt, J=12.4 Hz, 6.2 Hz, 1H), 1.44 (d, J=6.2 Hz, 6H). Minor solvent impurities.

¹³C-NMR,APT (76 MHz, CDCl₃): δ=164.7, 155.2, 149.1, 138.5, 138.1, 133.8, 133.3, 130.9, 129.2, 126.4, 124.0, 123.2, 69.8, 22.0.

HRMS (EI-MS) for C₁₅H₁₃Cl₂NO₂: calcd=309.0323, found=309.0326, Δm=1.0 ppm.

m.p.=74-76° C.

### Example 29: NG-487

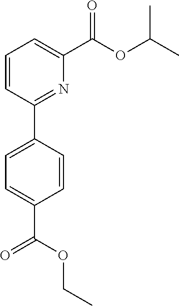 [SMILES: CCOC(=O)c1ccc(-c2cccc(C(=O)OC(C)C)n2)cc1]

The coupling of isopropyl 6-bromopicolinate with 4-ethoxyphenylboronic acid was performed following the general procedure SC1 with the modification that 2.22 eq CsF were used.

Yield=103.9 mg colorless oil (0.332 mmol, 80%).

R_{f}=0.22 (cyclohexane/EtOAc=7+1; UV, KMnO₄).

¹H-NMR (300 MHz, CDCl₃): δ=8.28-7.82 (m, 7H), 5.46-5.21 (m, 1H), 4.41 (q, J=7.1 Hz, 2H), 1.43 (dd, J=9.6 Hz, 6.8 Hz, 9H).

¹³C-NMR,APT (76 MHz, CDCl₃): δ=166.5, 164.8, 156.5, 149.1, 142.6, 137.9, 131.3, 130.2, 127.2, 124.0, 123.8, 69.7, 61.3, 22.0, 14.5.

HRMS (EI-MS) for C₁₈H₁₉NO₄: calcd=313.1314, found=313.1324, Δm=1.4 ppm.

m.p.=56-62° C.

### Example 30: NG-488

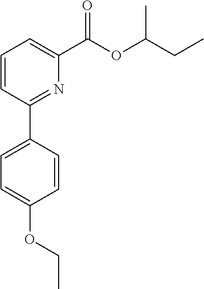 [SMILES: CCOc1ccc(-c2cccc(C(=O)OC(C)CC)n2)cc1]

The esterification of NG-482 with butan-2-ol was performed following the general procedure ES2.

Yield=14.6 mg colorless solid (0.0488 mmol, 12%).

R_{f}=0.28 (cyclohexane/EtOAc=7+1; UV).

¹H-NMR (300 MHz, CDCl₃): δ=8.05 (d, J=8.7 Hz, 2H), 7.93 (dd, J=8.8 Hz, 4.1 Hz, 1H), 7.82 (d, J=3.7 Hz, 2H), 6.99 (d, J=8.7 Hz, 2H), 5.26-5.08 (m, 1H), 4.10 (q, J=6.9 Hz, 2H), 1.94-1.63 (m, 2H), 1.52-1.32 (m, 6H), 1.02 (t, J=7.4 Hz, 3H).

¹³C-NMR,APT (76 MHz, CDCl₃): δ=165.2, 160.4, 157.3, 148.7, 137.5, 131.1, 128.6, 122.5 (2 carbon atoms according to HSQC), 114.8, 63.7, 29.1, 19.6, 14.9, 9.9.

HRMS (EI-MS) for C₁₈H₂₁NO₃: calcd=299.1521, found=299.1529, Δm=2.7 ppm.

m.p.=49-51° C.

### Example 31: NG-489

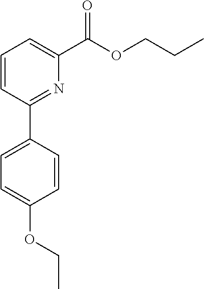 [SMILES: CCCOC(=O)c1cccc(-c2ccc(OCC)cc2)n1]

The esterification of NG-482 with n-propanol was performed following the general procedure ES2.

Yield=16.6 mg colorless solid (0.0582 mmol, 13%).

R_{f}=0.25 (cyclohexane/EtOAc=7+1; UV).

¹H-NMR (300 MHz, CDCl₃): δ=8.03 (d, J=8.7 Hz, 2H), 7.97 (t, J=4.3 Hz, 1H), 7.83 (d, J=4.3 Hz, 2H), 6.99 (d, J=8.7 Hz, 2H), 4.38 (t, J=6.8 Hz, 2H), 4.10 (q, J=6.9 Hz, 2H), 1.85 (dt, J=14.3 Hz, 7.1 Hz, 2H), 1.44 (t, J=6.9 Hz, 3H), 1.06 (t, J=7.4 Hz, 3H).

¹³C-NMR,APT (76 MHz, CDCl₃): δ=165.7, 160.4, 157.3, 148.3, 137.6, 131.0, 122.7, 122.6, 114.9, 67.4, 63.7, 22.2, 14.9, 10.6.

HRMS (EI-MS) for C₁₇H₁₉NO₃: calcd 285.1365, found=285.1377, Δm=4.2 ppm.

m.p.=80-84° C.

### Example 32: NG-490

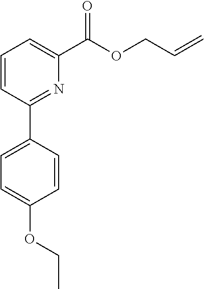 [SMILES: C=CCOC(=O)c1cccc(-c2ccc(OCC)cc2)n1]

The procedure is based on the synthesis of a similar substrate (Angew. Chem. Int. Ed. 2014, 53, 10536-10540)

A Schienk tube was dried under vacuum and was charged with 98.7 mg (0.406 mmol) NG-482, 2 mL anhydrous DMF, 55.2 mg (0.657 mmol) NaHCO₃, and 53 μL (0.613 mmol) allyl bromide. The mixture was stirred at 50° C. (oil-bath) overnight, until which time TLC indicated all starting material to be consumed. To the mixture were added 10 mL H₂O and the mixture was extracted with CH₂Cl₂ (4×10 mL. The combined organic layers were dried over MgSO₄, filtered and the solvent was removed under reduced pressure. The crude product was purified via column chromatography (cyclohexane/EtOAc=7+1) and 102.9 mg (0.363 mmol, 90%) of NG-490 were isolated as colorless solid.

R_{f}=0.27 (cyclohexane/EtOAc=7+1; UV, KMnO₄).

¹H-NMR (300 MHz, CDCl₃): δ=8.19-7.91 (m, 3H), 7.84 (d, J=4.2 Hz, 2H), 6.99 (d, J=8.7 Hz, 2H), 6.21-5.98 (m, 1H), 5.48 (dd, J=1.2 Hz, J=17.2 Hz, 1H), 5.33 (dd, J=1.0 Hz, J=10.4 Hz, 1H), 4.92 (d, J=5.6 Hz, 2H), 4.10 (q, J=6.9 Hz, 2H).

¹³C-NMR,APT (76 MHz, CDCl₃): δ=165.3, 160.45, 157.5, 148.0, 137.7, 132.2, 130.9, 128.7, 122.9, 122.8, 118.8, 114.9, 66.4, 63.7, 14.9.

HRMS (EI-MS) for C₁₇H₁₇NO₃: calcd=283.1208, found=283.1197, Δm=3.9 ppm.

m.p.=65-70° C.

### Example 33: NG-494

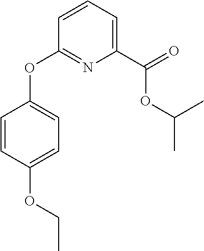 [SMILES: CCOc1ccc(Oc2cccc(C(=O)OC(C)C)n2)cc1]

A Schienk tube was dried under vacuum and was charged with 52.8 mg (0.382 mmol) 4-ethoxyphenol, 116.0 mg (0.475 mmol) isopropyl 6-bromopicolinate, 7.0 mg (36.8 μmol) CuI, 9.8 mg (79.6 μmol) picolinic acid, 166.9 mg (0.786 mmol) K₃PO₄, and 1.0 mL anhydrous DMSO. The mixture was stirred at 90° C. (oil-bath) overnight. Subsequently, 4 mL H₂O were added to the mixture and the mixture was extracted with EtOAc (4×4 mL). The combined organic layers were dried over MgSO₄, filtered and the solvent was removed under reduced pressure. The crude product was purified via preparative-HPLC (method A) and 38.6 mg (0.128 mmol, 34%) of NG-494 were isolated as colorless oil.

R_{f}=0.45 (cyclohexane/EtOAc=5+1; UV).

¹H-NMR (300 MHz, CDCl₃): δ=7.88-7.63 (m, 2H), 7.11 (d, J=8.9 Hz, 2H), 6.96-6.82 (m, 3H), 5.36-5.16 (m, 1H), 4.03 (q, J=6.9 Hz, 2H), 1.50-1.25 (m, 10H).

¹³C-NMR,APT (76 MHz, CDCl₃): δ=164.4, 164.1, 156.3, 147.3, 140.1, 122.2, 119.7, 115.6, 113.7, 69.6, 64.0, 22.0, 15.0. 1 carbon signal is missing maybe due to overlap.

HRMS (EI-MS) for C₁₇H₁₉NO₄: calcd=301.1314, found=301.1327, Δm=4.3 ppm.

### Example 34: NG-495

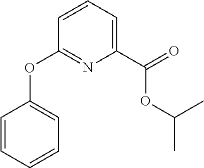 [SMILES: CC(C)OC(=O)c1cccc(Oc2ccccc2)n1]

A Schienk tube was dried under vacuum and was charged with 52.1 mg (0.554 mmol) phenol, 160.8 mg (0.659 mmol) isopropyl 6-bromopicolinate, 10.8 mg (56.7 μmol) CuI, 16.9 mg (137 μmol) picolinic acid, 236.4 mg (1.11 mmol) K₃PO₄, and 1 mL anhydrous DMSO. The mixture was stirred at 90° C. (oil-bath) overnight. Subsequently, 4 mL H₂O were added to the mixture and the mixture was extracted with EtOAc (4×4 mL). The combined organic layers were dried over MgSO₄, filtered and the solvent was removed under reduced pressure. The crude product was purified via preparative-HPLC (method A) and 43.0 mg (0.167 mmol, 30%) of NG-495 were isolated as colorless oil.

R_{f}=0.55 (cyclohexane/EtOAc=5+1; UV).

¹H-NMR (300 MHz, CDCl₃): δ=7.89-7.70 (m, 2H), 7.40 (t, J=7.8 Hz, 2H), 7.25-7.10 (m, 3H), 6.93 (d, J=7.9 Hz, 1H), 5.36-5.15 (m, 1H), 1.38 (d, J=6.2 Hz, 6H).

¹³C-NMR,APT (76 MHz, CDCl₃): δ=164.3, 163.5, 154.1, 147.3, 140.2, 129.9, 125.0, 121.0, 120.0, 114.4, 69.6, 21.9.

HRMS (EI-MS) for C₁₅H₁₅NO₃: calcd=257.1052, found=257.1059, Δm=2.7 ppm.

### Example 35: NG-497

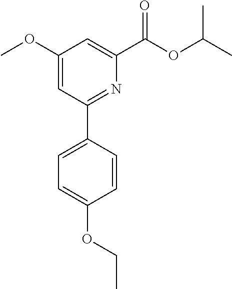 [SMILES: CCOc1ccc(-c2cc(OC)cc(C(=O)OC(C)C)n2)cc1]

A Schlenk tube was charged with 75.0 mg (0.261 mmol) NG-470 and 1.5 mL MeOH. Subsequently, 183 μL of a 2 M aqueous NaOH (0.366 mmol) were added and the mixture was stirred overnight at 80° C. (oil-bath), after which time TLC analysis indicated all starting material to be consumed. The solvent was removed under reduced pressure and 5 mL H₂O were added. Using 37m % HCl, the aqueous layer was acidified to pH=1 and was extracted with EtOAc (5×5 mL). Subsequently, the combined organic layers were dried over MgSO₄, filtered, and the solvent was removed under reduced pressure and 45.8 mg of a gum-like substance were isolated.

In a round-bottom flask, equipped with an Ar-inlet were placed 36.5 mg of the crude gum-like substance, 1 mL anhydrous CH₂Cl₂, 76.8 mg (0.401 mmol) EDC*HCl, 5.1 mg (41.7 μmol) DMAP, and 32.9 μL (0.427 mmol) i-PrOH. The mixture was stirred at rt overnight, until which time TLC indicated all starting material to be consumed. The solvent was removed under reduced pressure and the crude product was purified via column chromatography (cyclohexane/EtOAc=5+1) and 22.3 mg (0.0707 mmol, 34% calc. over 2 steps) of NG-497 were isolated as colorless solid.

R_{f}=0.29 (cyclohexane/EtOAc=5+1; UV).

¹H-NMR (300 MHz, CDCl₃): δ=8.00 (d, J=8.6 Hz, 2H), 7.58-7.45 (m, 1H), 7.35-7.25 (m, 1H), 6.97 (d, J=8.6 Hz, 2H), 5.42-5.20 (m, 1H), 4.09 (q, J=6.9 Hz, 2H), 3.96 (s, 3H), 1.54-1.28 (m, 9H).

¹³C-NMR,APT (76 MHz, CDCl₃): δ=167.4, 164.8, 160.5, 158.8, 149.9, 130.7, 128.8, 114.7, 109.1, 108.5, 69.8, 63.7, 55.8, 22.0, 14.9.

HRMS (EI-MS) for C₁₈H₂₁NO₄: calcd=315.1471, found=315.1475, Δm=1.3 ppm.

m.p.=79-81° C.

### Reference Compound NG-500

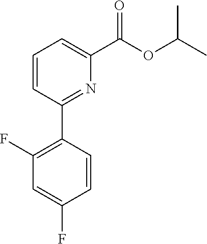 [SMILES: CC(C)OC(=O)c1cccc(-c2ccc(F)cc2F)n1]

The coupling of isopropyl 6-bromopicolinate was performed following the general procedure SC1.

Yield=102.1 mg brown oil (0.368 mmol, 94%).

R_{f}=0.52 (cyclohexane/EtOAc=5+1; UV, KMnO₄).

¹H-NMR (300 MHz, CDCl₃): δ=8.32-7.54 (m, 4H), 7.13-6.78 (m, 2H), 5.45-5.16 (m, 1H), 1.38 (m, 6H).

¹³C-NMR,APT (76 MHz, CDCl₃) δ 165.6-165.1 (m), 164.7, 152.7, 148.9, 139.1, 137.6, 132.9 (dd, J=9.7 Hz, 4.3 Hz), 131.6, 127.2 (d, J=10.5 Hz), 124.0, 123.6, 112.4 (d, J=3.6 Hz), 112.1 (d, J=3.6 Hz), 104.8, 104.4), 104.1, 70.2, 69.7, 22.0, 21.9.

HRMS (EI-MS) for C₁₅H₁₃F2NO₂: calcd=277.0914, found=277.0905, Δm=3.2 ppm.

### Example 36: NG-510

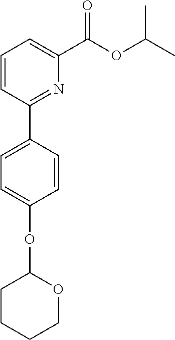 [SMILES: CC(C)OC(=O)c1cccc(-c2ccc(OC3CCCCO3)cc2)n1]

The coupling of isopropyl 6-bromopicolinate with 4-(2-tetrahydropyranyloxy)benzeneboronic acid was performed following the general procedure SC1.

Yield=150.5 mg colorless solid (0.441 mmol, 91%).

R_{f}=0.24 (cyclohexane/EtOAc=7+1; UV).

¹H-NMR (300 MHz, CDCl₃): δ=8.11-7.73 (m, 5H), 7.14 (d, J=8.7 Hz, 1.1H), 6.96 (d, J=8.5 Hz, 0.8H), 5.55-5.45 (m, 0.6H), 5.40-5.25 (m, 1H), 5.00-4.80 (m, 0.3H), 4.10-3.80 (m, 1H), 3.69-3.36 (m, 1H), 2.11-1.50 (m, 6H), 1.43 (d, J=6.2 Hz, 6H).

¹³C-NMR,APT (76 MHz, CDCl₃, major conformer): 6=165.2, 158.1, 157.4, 148.6, 137.6, 132.0, 128.6, 122.8, 122.7, 116.7, 96.4, 69.5, 62.2, 30.4, 25.3, 22.0, 18.8. The occurrence of “double-peaks” hints at the occurrence of both diastereomers. According to ¹H-NMR conformer ratio is 0.58:0.42.

¹³C-NMR,APT (76 MHz, CDCl₃, minor conformer): 6=165.0, 158.4, 157.6, 148.0, 138.0, 130.3, 128.9, 123.1, 122.6, 116.1, 94.8, 69.9, 63.1, 30.8, 25.6, 22.0, 19.9.

HRMS (EI-MS) for C₂₀H₂₃NO₄: calcd=341.1627, found=341.1628, Δm=0.3 ppm.

m.p.=105-107° C.

### Example 37: NG-512

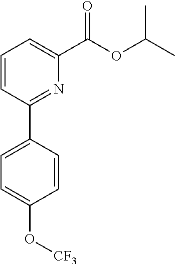 [SMILES: CC(C)OC(=O)c1cccc(-c2ccc(OC(F)(F)F)cc2)n1]

The coupling of isopropyl 6-bromopicolinate with 4-(trifluoromethoxy)phenylboronic acid was performed following the general procedure SC1.

Yield=126 mg colorless solid (0.387 mmol, 93%)

R_{f}=0.34 (cyclohexane/EtOAc=7+1; UV).

¹H-NMR (500 MHz, CDCl₃): δ=8.17-8.08 (m, 2H), 8.03 (dd, J=7.1 Hz, 1.3 Hz, 1H), 7.93-7.84 (m, 2H), 7.32 (d, J=8.3 Hz, 2H), 5.34 (hept, J=6.3 Hz, 1H), 1.44 (d, J=6.3 Hz, 6H).

¹³C-NMR,APT (126 MHz, CDCl₃): δ=164.9, 156.3, 150.4, 149.0, 137.9, 137.3, 128.9, 123.6, 123.2, 122.3, 120.7 (q, J=256.7 Hz), 121.2, 69.7, 22.0.

HRMS (EI-MS) for C₁₆H₁₄F3NO₃: calcd=325.0926, found=325.0936, Δm=3.1 ppm.

m.p.=43-47° C.

### Example 38: NG-513

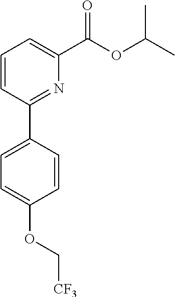 [SMILES: CC(C)OC(=O)c1cccc(-c2ccc(OCC(F)(F)F)cc2)n1]

The coupling of isopropyl 6-bromopicolinate with 4-(2,2,2-trifluoroethoxy)benzeneboronic acid was performed following the general procedure SC1 with the modification that 2.23 eq CsF were used.

Yield=116.3 mg slightly yellow solid (0.343 mmol, 84%)

R_{f}=0.34 (cyclohexane/EtOAc=5+1; UV).

¹H-NMR (300 MHz, CDCl₃): δ=8.08 (d, J=8.6 Hz, 2H), 8.04-7.95 (m, 1H), 7.91-7-79 (m, 2H), 7.04 (d, J=8.6 Hz, 2H), 5.45-5.22 (m, 1H), 4.41 (q, J=8.0 Hz, 2H), 1.44 (d, J=6.2 Hz, 6H).

¹³C-NMR,APT (76 MHz, CDCl₃): δ=165.0, 158.6, 156.8, 148.7, 137.8, 133.1, 128.9, 125.3, 123.0, 122.8, 121.6, 115.2, 69.6, 66.0 (q, J=35.7 Hz), 22.0.

HRMS (EI-MS) for C₁₇H₁₆F₃NO₃: calcd=339.1082, found=339.1089, Δm=2.1 ppm.

m.p.=70-75° C.

### Example 39: NG-527

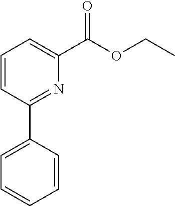 [SMILES: CCOC(=O)c1cccc(-c2ccccc2)n1]

The coupling of ethyl 6-bromopyridine-2-carboxylate with phenylboronic acid was performed following the general procedure SC1.

Yield=88.4 mg slightly yellowish solid (0.818 mmol, 93%).

R_{f}=0.44 (cyclohexane/EtOAc=5+1; UV).

¹H NMR (300 MHz, CDCl₃) δ 8.15-7.95 (m, 3H), 7.95-7.80 (m, 2H), 7.59-7.35 (m, 3H), 4.49 (q, J=7.1 Hz, 2H), 1.46 (t, J=7.1 Hz, 3H).

¹³C-NMR,APT (76 MHz, CDCl₃): δ=165.6, 157.7, 148.5, 138.6, 137.7, 129.5, 128.9, 127.3, 123.6, 123.4, 61.9, 14.4.

HRMS (EI-MS) for C₁₄H₁₃NO₂: calcd=227.0946, found=227.0935, Δm=4.8 ppm.

m.p.=42-46° C.

### Example 40: NG-530

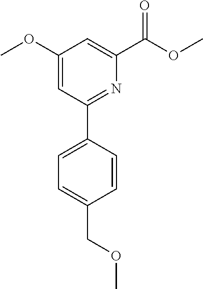

The coupling of methyl 6-chloro-4-methoxypicolinate with 4-(methoxymethyl)benzeneboronic acid was performed following the general procedure SC2 with the modification that 2.22 eq of CsF were used.

Yield=90.3 mg colorless solid (0.314 mmol, 63%).

R_{f}=0.29 (cyclohexane/EtOAc=3+1; UV).

¹H-NMR (300 MHz, CDCl₃): δ=7.99 (d, J=8.1 Hz, 2H), 7.61 (d, J=2.1 Hz, 1H), 7.43 (d, J=8.0 Hz, 2H), 7.36 (d, J=2.1 Hz, 1H), 4.51 (s, 2H), 4.06-3.91 (2×s, 6H), 3.39 (s, 3H).

¹³C-NMR,APT (76 MHz, CDCl₃): δ=167.4, 166.2, 159.3, 149.8, 139.7, 138.1, 128.1, 127.4, 110.0, 109.5, 74.4, 58.2, 55.7, 53.0.

HRMS (EI-MS) for C₁₅H₁₇NO₄: calcd=287.1158, found=287.1149, Δm=3.1 ppm.

m.p.=65-68° C.

### Example 41: NG-531

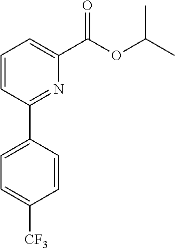 [SMILES: CC(C)OC(=O)c1cccc(-c2ccc(C(F)(F)F)cc2)n1]

The coupling of isopropyl 6-bromopicolinate with 4-trifluoromethylphenylboronic acid was performed following the general procedure SC1.

Yield=75.7 mg colorless crystals (0.245 mmol, 57%).

R_{f}=0.39 (cyclohexane/EtOAc=5+1; UV).

¹H-NMR (500 MHz, CDCl₃): δ=8.21 (d, J=8.2 Hz, 2H), 8.07 (dd, J=8.7 Hz, 4.4 Hz, 1H), 7.95-7.91 (m, 2H), 7.74 (d, J=8.2 Hz, 2H), 5.35 (hept, J=6.3 Hz, 1H), 1.45 (d, J=6.3 Hz, 6H).

¹³C-NMR (126 MHz, CDCl₃): δ=164.8, 156.1, 149.2, 141.9, 138.0, 131.4 (q, J=32.4 Hz), 127.6, 125.9 (q, J=3.8 Hz), 124.3 (q, J=272.2 Hz, only 2 signal visible), 124.1, 123.6, 69.7, 22.0.

HRMS (EI-MS) for C₁₆H₁₄F₃NO₂: calcd=309.0977, found=309.0981, Δm=1.3 ppm.

m.p.=98-99° C.

### Example 42: NG-534

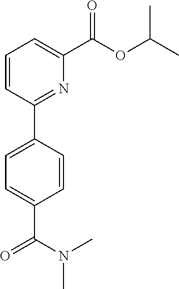 [SMILES: CC(C)OC(=O)c1cccc(-c2ccc(C(=O)N(C)C)cc2)n1]

The coupling of isopropyl 6-bromopicolinate with 4-(dimethylcarbamoyl)phenylboronic acid was performed following the general procedure SC1.

Yield=118.7 mg brown, cloudy oil (0.380 mmol, 90%).

R_{f}=0.39 (cyclohexane/EtOAc=1+2; UV).

¹H-NMR (300 MHz, CDCl₃): δ=8.05-7.91 (m, 3H), 7.90-7.79 (m, 2H), 7.29 (d, J=8.0 Hz, 2H), 5.42-5.24 (m, 1H), 2.64 (t, J=7.5 Hz, 2H), 1.75-1.60 (m, 2H), 1.44 (d, J=6.2 Hz, 6H), 0.95 (t, J=7.3 Hz, 3H).

¹³C-NMR,APT (76 MHz, CDCl₃): δ=165.1, 157.8, 148.7, 144.4, 137.6, 136.2, 129.1, 127.2, 123.1, 123.0, 69.5, 37.9, 24.6, 22.0, 13.9.

HRMS (EI-MS) for C₁₈H₂₀N₂O₃: calcd=312.1474, found=312.1470, Δm=1.3 ppm.

### Example 43: NG-536

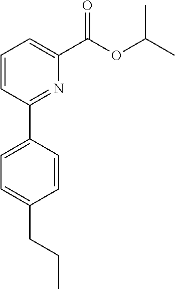 [SMILES: CCCc1ccc(-c2cccc(C(=O)OC(C)C)n2)cc1]

The coupling of isopropyl 6-bromopicolinate with 4-propylphenylboronic acid was performed following the general procedure SC1.

Yield=112.8 mg colorless solid (0.398 mmol, 97%).

R_{f}=0.38 (cyclohexane/EtOAc=10+1; UV).

¹H-NMR (300 MHz, CDCl₃): δ=8.08-7.92 (m, 3H), 7.92-7.79 (m, J=6.3 Hz, 2H), 7.28 (d, J=8.0 Hz, 2H), 5.42-5.25 (m, 1H), 2.64 (t, J=7.5 Hz, 2H), 1.68 (dd, J=14.8 Hz, 7.6 Hz, 2H), 1.44 (d, J=6.2 Hz, 6H), 0.95 (t, J=7.3 Hz, 3H).

¹³C-NMR,APT (76 MHz, CDCl₃): δ=165.1, 157.8, 148.7, 144.4, 137.6, 136.2, 129.1, 127.2, 123.1, 123.0, 69.5, 37.9, 24.6, 22.0, 13.9.

HRMS (EI-MS) for C₁₈H₂₁NO₂: calcd=283.1572, found=283.1572, Δm=0 ppm.

m.p.=104-107° C.

### Example 4: NG-545

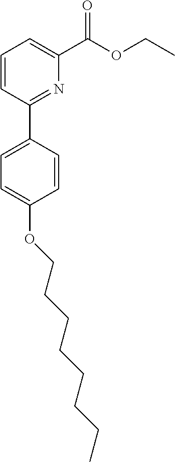 [SMILES: CCCCCCCCOc1ccc(-c2cccc(C(=O)OCC)n2)cc1]

The coupling of ethyl-6-bromopicolinate with 4-octoxyphenylboronic acid was performed following the general procedure SC2 with the modification that KF was used instead of CsF.

Yield=95.0 mg slightly yellow solid (0.267 mmol, 58%).

R_{f}=0.37 (cyclohexane/EtOAc=7+1; UV).

¹H-NMR (300 MHz, CDCl₃): δ=8.12-7.91 (m, 3H), 7.83 (d, J=4.3 Hz, 2H), 6.99 (d, J=8.7 Hz, 2H), 4.48 (q, J=7.1 Hz, 2H), 4.01 (t, J=6.5 Hz, 2H), 1.89-1.71 (m, 2H), 1.57-1.20 (m, 14H), 0.98-0.80 (m, 3H).

¹³C-NMR,APT (76 MHz, CDCl₃): δ=165.7, 160.6, 157.5, 148.3, 137.6, 131.0, 128.6, 122.8, 122.6, 114.9, 68.3, 61.9, 31.9, 29.5, 29.4, 26.2, 22.8, 14.5, 14.2.

HRMS (EI-MS) for C₂₂H₂₉NO₃: calcd=355.2148, found=355.2144, Δm=1.2 ppm.

m.p.=84-86° C.

### Example 45: NG-550

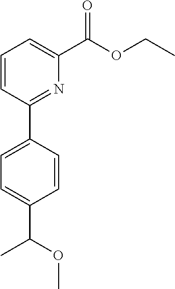 [SMILES: CCOC(=O)c1cccc(-c2ccc(C(C)OC)cc2)n1]

The coupling of ethyl-6-bromopicolinate with [4-(1-methoxyethyl)phenyl]boronic acid was performed following the general procedure SC1 with the modification that 2.29 eq CsF were used.

Yield=108.9 mg clear, slightly brown oil (0.382 mmol, 88%).

R_{f}=0.31 (cyclohexane/EtOAc=5+1; UV).

¹H NMR (300 MHz, CDCl₃) δ 8.13-7.95 (m, 3H), 7.88 (d, J=3.9 Hz, 2H), 7.42 (d, J=8.2 Hz, 2H), 4.49 (q, J=7.1 Hz, 2H), 4.36 (q, J=6.4 Hz, 1H), 3.24 (s, 3H), 1.54-1.36 (m, 6H).

¹³C-NMR,APT (76 MHz, CDCl₃): δ=165.6, 157.7, 148.5, 138.0, 137.8, 127.6, 126.8, 123.6, 123.3, 79.5, 62.0, 56.6, 24.0, 14.4. The occurrence of “double-peaks” hints at the occurrence of both diastereomers.

HRMS (EI-MS) for C₁₇H₁₉NO₃: calcd=285.1365, found=285.1355, Δm=3.5 ppm.

### Example: NG-556

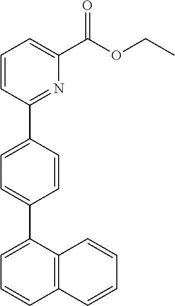 [SMILES: CCOC(=O)c1cccc(-c2ccc(-c3cccc4ccccc34)cc2)n1]

The coupling of ethyl-6-bromopicolinate with 4-(1-naphthyl)phenylboronic acid was performed following the general procedure SC1.

Yield=137.9 mg colorless sticky gum (0.390 mmol, 89%).

R_{f}=0.33 (cyclohexane/EtOAc=7+1; UV).

¹H-NMR (300 MHz, CDCl₃): δ=8.21 (d, J=8.1 Hz, 2H), 8.09 (d, J=7.3 Hz, 1H), 8.04-7.82 (m, 5H), 7.64 (d, J=8.1 Hz, 2H), 7.59-7.40 (m, 4H), 4.52 (q, J=7.1 Hz, 2H), 1.49 (t, J=7.1 Hz, 3H). Minor solvent impurities.

¹³C-NMR,APT (76 MHz, CDCl₃): δ=165.6, 157.6, 148.6, 142.1, 139.8, 137.8, 137.7, 134.0, 131.7, 130.7, 128.5, 128.0, 127.3, 127.0, 126.3, 126.1, 126.0, 125.5, 123.6, 123.5, 62.0, 14.5.

HRMS (EI-MS) for C₂₄H₁₉NO₂: calcd=353.1416, found=353.1420, Δm=1.1 ppm.

### Example 47: NG-560

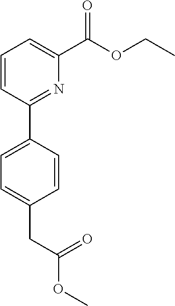 [SMILES: CCOC(=O)c1cccc(-c2ccc(CC(=O)OC)cc2)n1]

The coupling of ethyl-6-bromopicolinate with (4-methoxycarbonylmethyl)phenylboronic acid was performed following the general procedure SC1 with the modification that 2.23 eq CsF were used.

Yield=97.9 mg brown oil (0.327 mmol, 73%).

R_{f}=0.26 (cyclohexane/EtOAc=3+1; UV).

¹H-NMR (300 MHz, CDCl₃): δ=8.18-7.95 (m, 3H), 7.88 (d, J=4.4 Hz, 2H), 7.40 (d, J=8.1 Hz, 2H), 4.49 (q, J=7.1 Hz, 2H), 3.70, 3.69 (2×s, 5H), 1.46 (t, J=7.1 Hz, 3H).

¹³C-NMR,APT (76 MHz, CDCl₃): δ=171.9, 165.6, 157.4, 148.5, 137.8, 137.6, 135.5, 129.9, 127.6, 123.5, 123.4, 62.0, 52.3, 41.1, 14.5.

HRMS (EI-MS) for C₁₇H₁₇NO₄: calcd=299.1158, found=299.1149, Δm=3.0 ppm.

### Example 48: NG-561

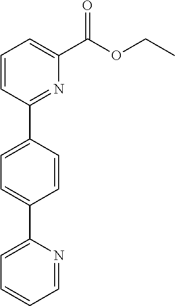 [SMILES: CCOC(=O)c1cccc(-c2ccc(-c3ccccn3)cc2)n1]

The coupling of ethyl-6-bromopicolinate with 4-(2-pyridiyl)phenylboronic acid was performed following the general procedure SC1.

Yield=46.4 mg slightly yellow solid (0.153 mmol, 34%).

R_{f}=0.17 (cyclohexane/EtOAc=3+1; UV).

¹H-NMR (300 MHz, CDCl₃): δ=8.64 (d, J=4.4 Hz, 1H), 8.21-7.62 (m, 9H), 7.17 (dd, J=8.0 Hz, 3.3 Hz, 1H), 4.42 (q, J=7.1 Hz, 2H), 1.39 (t, J=7.1 Hz, 3H). No referencing could be performed due to overlap.

¹³C-NMR,APT (76 MHz, CDCl₃): δ=165.6, 157.2, 156.9, 149.9, 148.5, 140.3, 139.0, 137.8, 136.9, 127.7, 127.4, 123.6, 123.5. 122.5, 120.8, 62.0, 14.4.

HRMS (EI-MS) for C₁₉H₁₆N₂O₂: calcd=304.1212, found=304.1206, Δm=2.0 ppm.

m.p.=130-132° C.

### Example 49: NG-562

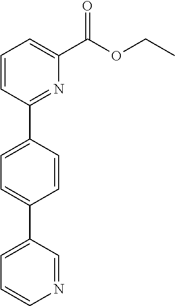 [SMILES: CCOC(=O)c1cccc(-c2ccc(-c3cccnc3)cc2)n1]

The coupling of ethyl-6-bromopicolinate with 4-(3-pyridyl)phenylboronic acid was performed following the general procedure SC1.

Yield=121.5 mg grey solid (0.412 mmol, 93%).

R_{f}=0.33 (cyclohexane/EtOAc=2+3; UV).

¹H-NMR (300 MHz, CDCl₃): δ=8.91 (s, 1H), 8.61 (d, J=3.6 Hz, 1H), 8.20 (d, J=8.2 Hz, 2H), 8.07 (dd, J=6.8 Hz, 1.3 Hz, 1H), 8.00-7.86 (m, 3H), 7.71 (d, J=8.2 Hz, 2H), 7.39 (dd, J=7.8 Hz, 4.8 Hz, 1H), 4.50 (q, J=7.1 Hz, 2H), 1.47 (t, J=7.1 Hz, 3H).

¹³C-NMR,APT (76 MHz, CDCl₃): δ=165.5, 157.0, 148.9, 148.6, 148.4, 138.9, 138.4, 137.9, 136.1, 134.4, 128.0. 127.6, 123.7, 123.6. 123.5, 62.0, 14.5.

HRMS (EI-MS) for C₁₉H₁₆N₂O₂: calcd=304.1212, found=304.1204, Δm=2.6 ppm.

m.p.=84-104° C.

### Example 50: NG-563

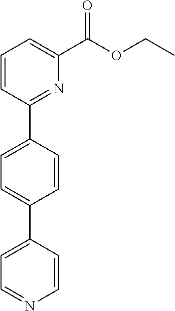 [SMILES: CCOC(=O)c1cccc(-c2ccc(-c3ccncc3)cc2)n1]

The coupling of ethyl-6-bromopicolinate with 4-(4-pyridyl)phenylboronic acid was performed following the general procedure SC1.

Yield=43.0 mg slightly yellow solid (0.327 mmol, 32%).

R_{f}=0.24 (cyclohexane/EtOAc=2+3; UV).

¹H-NMR (300 MHz, CDCl₃): δ=8.75-8.61 (m, 2H), 8.20 (d, J=8.3 Hz, 2H), 8.07 (dd, J=6.8 Hz, 1.7 Hz, 1H), 8.00-7.85 (m, 2H), 7.76 (d, J=8.3 Hz, 2H), 7.63-7.47 (m, 2H), 4.50 (q, J=7.1 Hz, 2H), 1.47 (t, J=7.1 Hz, 3H).

¹³C-NMR,APT (76 MHz, CDCl₃): δ=165.5, 156.8, 150.4, 148.6, 147.8, 139.2, 139.1, 137.9, 128.0, 127.5, 123.7, 123.6, 121.7, 62.0, 14.4.

HRMS (EI-MS) for C₁₉H₁₆N₂O₂: calcd=304.1212, found=304.1210, Δm=0.7 ppm.

m.p.=127-131° C.

### Example 51: NG-576

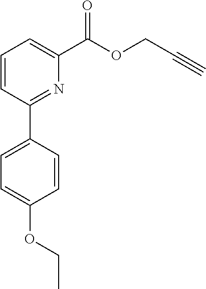 [SMILES: C#CCOC(=O)c1cccc(-c2ccc(OCC)cc2)n1]

The esterification of 6-bromopyridine-2-carboxylic acid (see also: NG-444/NG-482) with propargyl alcohol was performed following the general procedure ES1.

Yield=63.4 mg colorless solid (0.225 mmol, 68%).

R_{f}=0.31 (cyclohexane/EtOAc=5+1; UV).

¹H-NMR (300 MHz, CDCl₃): δ=8.15-7.92 (m, 3H), 7.91-7.76 (m, 2H), 6.97 (d, J=8.7 Hz, 2H), 5.00 (d, J=2.2 Hz, 2H), 4.07 (q, J=6.9 Hz, 2H), 2.54 (t, J=2.2 Hz, 1H), 1.42 (t, J=6.9 Hz, 3H).

¹³C-NMR,APT (76 MHz, CDCl₃): δ=164.8, 160.4, 157.5, 147.3, 137.6, 130.8, 128.6, 123.1, 123.0, 114.8, 77.6, 75.4, 63.6, 53.1, 14.9.

HRMS (EI-MS) for C₁₇H₁₅NO₃: calcd=281.1052, found=281.1039, Δm=4.6 ppm.

m.p.=76-78° C.

### Example 52: NG-577

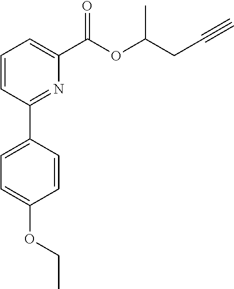 [SMILES: C#CCC(C)OC(=O)c1cccc(-c2ccc(OCC)cc2)n1]

The esterification of 6-bromopyridine-2-carboxylic acid (see also: NG-444/NG-482) with 4-pentyn-2-ol was performed following the general procedure ES1.

Yield=54.3 mg colorless oil (0.176 mmol, 53%).

R_{f}=0.34 (cyclohexane/EtOAc=5+1; UV).

¹H-NMR (300 MHz, CDCl₃): δ=8.04 (d, J=8.7 Hz, 2H), 7.95 (dd, J=5.2 Hz, 3.4 Hz, 1H), 7.89-7.80 (m, 2H), 6.99 (d, J=8.7 Hz, 2H), 5.42-5.25 (m, 1H), 4.10 (q, J=6.9 Hz, 2H), 2.81-2.54 (m, 2H), 2.05 (t, J=2.5 Hz, 1H), 1.53 (d, J=6.3 Hz, 3H), 1.44 (t, J=6.9 Hz, 3H).

¹³C-NMR,APT (76 MHz, CDCl₃): δ=164.8, 160.4, 157.5, 148.1, 137.6, 131.0, 128.6, 122.7 (2×), 114.8, 79.8, 70.8, 70.2, 63.7, 25.8, 19.3, 14.9.

HRMS (EI-MS) for C₁₉H₁₉NO₃: calcd=309.1365, found=309.1358, Δm=2.3 ppm.

### Intermediate NG-581 (Also Referred to as TSch-39)

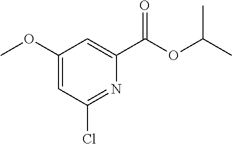

The esterification of 6-chloro-4-methoxypyridine-2-carboxylic acid with isopropanol was performed following the general procedure ES1.

Yield=555.5 mg colorless solid (2.42 mmol, 60%).

R_{f}=0.32 (cyclohexane/EtOAc=5+1; UV).

¹H-NMR (300 MHz, CDCl₃): δ=7.54 (d, J=1.7 Hz, 1H), 6.96 (d, J=1.8 Hz, 1H), 5.45-5.10 (m, 1H), 3.91 (s, 3H), 1.40, 1.38 (2 s, 6H).

¹³C-NMR,APT (76 MHz, CDCl₃): δ=168.0, 163.6, 152.8, 149.9, 112.4, 111.5, 70.3, 56.2, 21.9.

HRMS (EI-MS) for C₁₀H₁₂ClNO₃: calcd=229.0506, found=229.0500, Δm=2.6 ppm.

m.p.=62-64° C.

### Example 53: NG-582

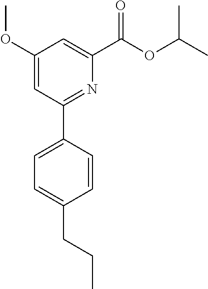 [SMILES: CCCc1ccc(-c2cc(OC)cc(C(=O)OC(C)C)n2)cc1]

The coupling of NG-581 with 4-propylphenylboronic acid was performed following the general procedure SC1.

Yield=369.7 mg slightly yellowish oil (1.18 mmol, 72%).

R_{f}=0.33 (cyclohexane/EtOAc=5+1; UV).

¹H-NMR (300 MHz, CDCl₃): δ=7.99 (d, J=8.1 Hz, 2H), 7.57 (d, J=2.1 Hz, 1H), 7.38 (d, J=2.1 Hz, 1H), 7.30 (d, J=7.7 Hz, 2H), 5.47-5.25 (m, 1H), 3.99 (s, 3H), 2.67 (t, J=7.5 Hz, 2H), 1.70 (dd, J=14.9 Hz, 7.3 Hz, 2H), 1.47 (d, J=6.2 Hz, 6H), 0.98 (t, J=7.3 Hz, 3H).

¹³C-NMR,APT (76 MHz, CDCl₃): δ=167.2, 165.1, 159.4, 150.4, 144.4, 136.3, 129.0, 127.2, 109.3, 109.0, 69.6, 55.6, 37.9, 24.6, 22.0, 13.9.

HRMS (EI-MS) for C₁₉H₂₃NO₃: calcd=313.1678, found=313.1673, Δm=1.6 ppm.

### Example 54: NG-584

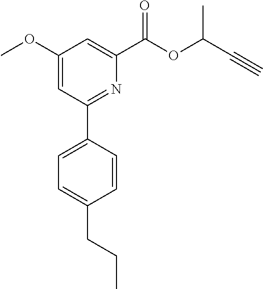 [SMILES: C#CC(C)OC(=O)c1cc(OC)cc(-c2ccc(CCC)cc2)n1]

A Schlenk tube was charged with 328.6 mg (1.05 mmol) NG-582 and 10 mL MeOH. Subsequently, 1.1 mL of a 2 M aqueous NaOH (2.2 mmol) were added and the mixture was stirred overnight at 80° C. (oil-bath), after which time TLC analysis indicated all starting material to be consumed. The solvent was removed under reduced pressure and 25 mL H₂O were added. Using 37m % HCl, the aqueous layer was acidified to pH=1 and was extracted with EtOAc (5×25 mL). Subsequently, the combined organic layers were dried over Na₂SO₄, filtered, and the solvent was removed under reduced pressure and 109.0 mg of a yellow, sticky oil were isolated. The esterification 39.0 mg of the crude material with 3-butyn-2-ol was performed following the general procedure ES1 with the modification that 1.2 mL THF were used and additional column chromatographies and ACN/hexane extractions were performed for purification.

Yield=15.9 mg yellow oil (47.2 μmol, 13% over 2 steps).

R_{f}=0.21 (cyclohexane/EtOAc=10+1; UV).

¹H-NMR (300 MHz, CDCl₃): δ=7.96 (d, J=8.1 Hz, 2H), 7.58 (d, J=2.1 Hz, 1H), 7.36 (d, J=2.1 Hz, 1H), 7.32-7.18 (m, 2H), 5.82-5.61 (m, 1H), 3.96 (s, 3H), 2.64 (t, J=7.5 Hz, 2H), 2.51 (d, J=2.0 Hz, 1H), 1.77-1.59 (m, 5H), 0.95 (t, J=7.3 Hz, 3H).

¹³C-NMR,APT (76 MHz, CDCl₃): δ=167.3, 164.4, 159.5, 149.3, 144.6, 136.0, 129.0, 127.3, 109.7, 109.5, 82.1, 73.6, 61.7, 55.8, 37.9, 24.6, 21.4, 13.9.

HRMS (EI-MS) for C₂₀H₂₁NO₃: calcd=323.1521, found=323.1503, Δm=5.6 ppm.

### Reference Compound NG-587

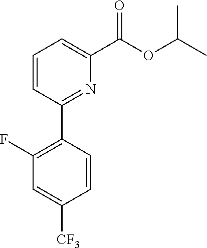 [SMILES: CC(C)OC(=O)c1cccc(-c2ccc(C(F)(F)F)cc2F)n1]

The coupling of isopropyl 6-bromopicolinate with 2-fluoro-4-(trifluoromethyl)phenylboronic acid was performed following the general procedure SC1.

Yield=111.2 mg colorless solid (0.340 mmol, 82%).

R_{f}=0.28 (cyclohexane/EtOAc=10+1; UV).

¹H-NMR (500 MHz, CDCl₃): δ=8.31 (t, J=7.9 Hz, 1H), 8.09 (d, J=7.7 Hz, 1H), 8.00 (d, J=7.9 Hz, 1H), 7.92 (t, J=7.8 Hz, 1H), 7.56 (d, J=8.2 Hz, 1H), 7.44 (d, J=11.0 Hz, 1H), 5.35 (hept, J=6.3 Hz, 1H), 1.44 (d, J=6.3 Hz, 6H).

¹³C-NMR (126 MHz, CDCl₃): δ=164.4, 161.2, 159.2, 151.9 (d, J=2.5 Hz), 149.1, 137.5, 132.8 (dq, J=8.3 Hz, J=33.4 Hz), 132.4 (d, J=3.3 Hz) 130.0 (d, J=11.4 Hz), 127.4 (d, J=10.4 Hz), 124.2, 123.2 (dq, J=2.5 Hz, J=272.8 Hz), 121.5 (q, J=3.8 Hz), 113.7 (dq, J=26.5 Hz, 3.8 Hz), 69.6, 21.9

HRMS (EI-MS) for C₁₆H₁₃F₄NO₂: calcd=327.0883, found=327.0883, Δm=0 ppm.

m.p.=96-97° C.

### Example 55: NG-590

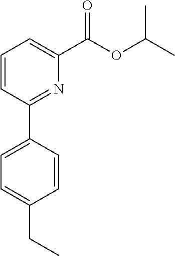 [SMILES: CCc1ccc(-c2cccc(C(=O)OC(C)C)n2)cc1]

The coupling of isopropyl 6-bromopicolinate with 4-ethylphenylboronic acid was performed following the general procedure SC1.

Yield=99.4 mg colorless solid (0.369 mmol, 89%).

R_{f}=0.32 (cyclohexane/EtOAc=10+1; UV).

¹H-NMR (300 MHz, CDCl₃): δ=8.09-7.92 (m, 3H), 7.91-7.79 (m, 2H), 7.31 (d, J=8.0 Hz, 2H), 5.47-5.22 (m, 1H), 2.71 (q, J=7.5 Hz, 20H), 1.44 (d, J=6.2 Hz, 6H), 1.27 (t, J=7.6 Hz, 3H).

¹³C-NMR,APT (76 MHz, CDCl₃): δ=165.1, 157.7, 148.7, 145.9, 137.6, 136.1, 128.4, 127.3, 123.1, 123.1, 69.5, 28.8, 22.0, 15.6.

HRMS (EI-MS) for C₁₇H₁₉NO₂: calcd=269.1416, found=269.1412, Δm=1.5 ppm.

m.p.=82-84° C.

### Example 56: NG-592

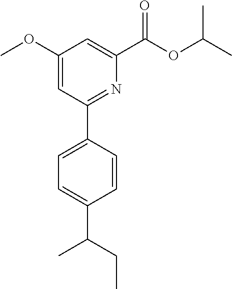 [SMILES: CCC(C)c1ccc(-c2cc(OC)cc(C(=O)OC(C)C)n2)cc1]

The coupling of NG-581 with 4-(sec-butyl)benzeneboronic acid was performed following the general procedure SC1.

Yield=75.0 mg slightly brownish oil (0.229 mmol, 79%).

R_{f}=0.36 (cyclohexane/EtOAc=7+1; UV).

¹H-NMR (300 MHz, CDCl₃): δ=7.96 (d, J=8.1 Hz, 2H), 7.54 (d, J=2.1 Hz, 1H), 7.34 (d, J=2.1 Hz, 1H), 7.28 (d, J=8.8 Hz, 2H), 5.42-5.22 (m, 1H), 3.96 (s, 3H), 2.75-2.55 (m, 1H), 1.7-1.54 (m, 2H), 1.43 (d, J=6.2 Hz, 6H), 1.26 (t, J=7.0 Hz, 3H), 0.83 (t, J=7.4 Hz, 3H).

¹³C-NMR,APT (76 MHz, CDCl₃): δ=167.2, 165.2, 159.5, 150.4, 149.3, 136.6, 127.6, 127.3, 109.3, 109.1, 69.6, 55.7, 41.7, 31.2, 22.0, 22.0, 12.3.

HRMS (EI-MS) for C₂₀H₂₅NO₃: calcd=327.1834, found=327.1832, Δm=0.6 ppm.

### Example 57: NG-593

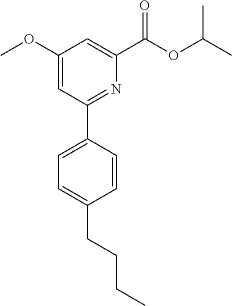 [SMILES: CCCCc1ccc(-c2cc(OC)cc(C(=O)OC(C)C)n2)cc1]

The coupling of NG-581 with 4-butylphenylboronic acid was performed following the general procedure SC1. An additional preparative-HPLC (method B) was performed for purification.

Yield=30.8 mg colorless oil (0.0941 mmol, 42%).

R_{f}=0.36 (cyclohexane/EtOAc=7+1; UV).

¹H-NMR (300 MHz, CDCl₃): δ=7.95 (d, J=8.0 Hz, 2H), 7.54 (d, J=2.0 Hz, 1H), 7.34 (d, J=2.0 Hz, 1H), 7.27 (d, J=7.9 Hz, 2H), 5.42-5.21 (m, 1H), 3.95 (s, 3H), 2.66 (t, J=7.6 Hz, 2H), 1.70-1.52 (m, 2H), 1.52-1.27 (m, 8H), 0.93 (t, J=7.3 Hz, 3H).

¹³C-NMR,APT (76 MHz, CDCl₃): δ=167.2, 165.1, 159.4, 150.4, 144.6, 136.3, 128.9, 127.2, 109.3, 109.0, 69.6, 55.6, 35.5, 33.6, 22.4, 22.0, 14.1.

HRMS (EI-MS) for C₂₀H₂₅NO₃: calcd=327.1834, found=327.1830, Δm=1.2 ppm.

### Example 58: NG-594

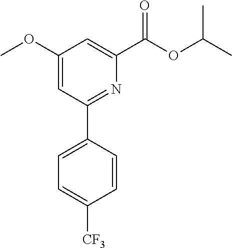 [SMILES: COc1cc(C(=O)OC(C)C)nc(-c2ccc(C(F)(F)F)cc2)c1]

The coupling of isopropyl 6-chloro-4-methoxypicolinate with 4-trifluoromethylphenylboronic acid was performed following the general procedure SC1.

Yield=48.5 mg colorless solid (0.143 mmol, 53%).

R_{f}=0.54 (cyclohexane/EtOAc=3+1; UV, KMnO₄).

¹H-NMR (500 MHz, CDCl₃): δ=8.16 (d, J=8.1 Hz, 2H), 7.72 (d, J=8.1 Hz, 2H), 7.61 (d, J=1.9 Hz, 1H), 7.39 (d, J=1.9 Hz, 1H), 5.33 (hept, J=6.2 Hz, 1H), 3.98 (s, 3H), 1.44 (d, J=6.3 Hz, 6H).

¹³C-NMR (126 MHz, CDCl₃): δ=167.5, 164.8, 157.7, 150.8, 142.1, 131.4 (q, J=32.5 Hz), 127.7, 125.4 (q, J=3.8 Hz), 124.3 (q, J=272.1), 110.0, 110.0, 69.9, 55.8, 22.0.

HRMS (EI-MS) for C₁₇H₁₆F3NO₃: calcd=339.1082, found=339.1090, Δm=2.4 ppm.

m.p.=55-56° C.

### Example 59: NG-595

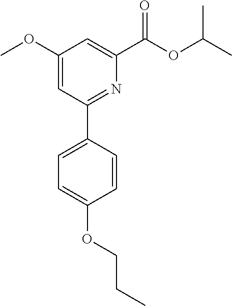 [SMILES: CCCOc1ccc(-c2cc(OC)cc(C(=O)OC(C)C)n2)cc1]

The coupling of TSch39 (see NG-581) with 4-propoxyphenylboronic acid was performed following the general procedure SC1. An additional preparative-HPLC (method B) was performed for purification.

Yield=37.2 mg colorless solid (0.113 mmol, 46%).

R_{f}=0.33 (cyclohexane/EtOAc=5+1; UV).

¹H-NMR (300 MHz, CDCl₃): δ=8.00 (d, J=8.7 Hz, 2H), 7.50 (d, J=2.0 Hz, 1H), 7.30 (d, J=2.0 Hz, 1H), 6.97 (d, J=8.7 Hz, 2H), 5.45-5.20 (m, 1H), 4.10-3.83 (m, 5H), 1.93-1.73 (m, 2H), 1.43 (d, J=6.2 Hz, 6H), 1.05 (t, J=7.4 Hz, 3H).

¹³C-NMR,APT (76 MHz, CDCl₃): δ=167.2, 165.2, 160.5, 159.0, 150.3, 131.2, 128.6, 114.7, 108.9, 108.4, 69.7, 69.6, 55.6, 22.7, 22.0, 10.6.

HRMS (EI-MS) for C₁₉H₂₃NO₄: calcd=329.1627, found=329.1627, Δm=0 ppm.

m.p.=79-81° C.

### Example 60: NG-596

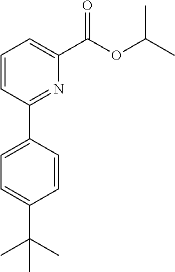 [SMILES: CC(C)OC(=O)c1cccc(-c2ccc(C(C)(C)C)cc2)n1]

The coupling of isopropyl 6-bromopicolinate with 4-tert-butylphenylboronic acid was performed following the general procedure SC1.

Yield=109.6 mg colorless solid (0.369 mmol, 87%).

R_{f}=0.44 (cyclohexane/EtOAc=7+1; UV).

¹H-NMR (300 MHz, CDCl₃): δ=8.13-7.92 (m, 3H), 7.92-7.80 (m, 2H), 7.51 (d, J=8.4 Hz, 2H), 5.44-5.24 (m, 1H), 1.44 (d, J=6.2 Hz, 6H), 1.36 (s, 9H).

¹³C-NMR,APT (76 MHz, CDCl₃): δ=165.1, 157.7, 152.8, 148.8, 137.5, 135.9, 127.1, 125.9, 123.1, 123.0, 69.4, 34.9, 31.4, 22.0.

HRMS (EI-MS) for C₁₉H₂₃NO₂: calcd=297.1729, found=297.1730, Δm=0.3 ppm.

m.p.=104-106° C.

### Example 61: NG-597

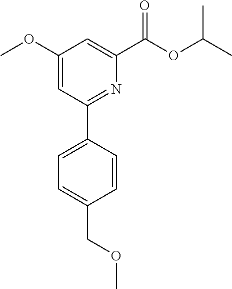 [SMILES: COCc1ccc(-c2cc(OC)cc(C(=O)OC(C)C)n2)cc1]

The coupling of isopropyl 6-chloro-4-methoxypicolinate with with 4-(methoxymethyl)benzeneboronic acid was performed following the general procedure SC2. An additional preparative-HPLC (method B) was performed for purification.

Yield=56.1 mg colorless oil (0.178 mmol, 55%).

R_{f}=0.25 (cyclohexane/EtOAc=4+1; UV).

¹H-NMR (300 MHz, CDCl₃): δ=8.03 (d, J=8.1 Hz, 2H), 7.55 (d, J=2.1 Hz, 1H), 7.43 (d, J=8.0 Hz, 2H), 7.36 (d, J=2.1 Hz, 1H), 5.41-5.22 (m, 6.2 Hz, 1H), 4.51 (s, 2H), 3.95 (s, 3H), 3.39 (s, 3H), 1.43 (d, J=6.2 Hz, 6H).

¹³C-NMR,APT (76 MHz, CDCl₃): δ=167.3, 165.0, 159.0, 139.7, 138.1, 128.0, 127.4, 109.5, 109.3, 74.4, 69.6, 58.2, 55.7, 22.0.

HRMS (EI-MS) for C₁₈H₂₁NO₄: calcd=315.1471, found=315.1495, Δm=7.6 ppm.

### Example 62: NG-598

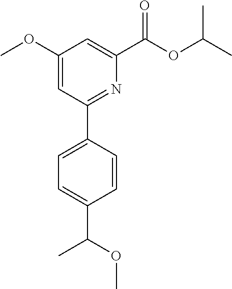 [SMILES: COc1cc(C(=O)OC(C)C)nc(-c2ccc(C(C)OC)cc2)c1]

The coupling of isopropyl 6-chloro-4-methoxypicolinate with [4-(1-methoxyethyl)phenyl]boronic acid was performed following the general procedure SC2. An additional preparative-HPLC (method B) was performed for purification.

Yield=67.0 mg colorless oil (0.203 mmol, 60%).

R_{f}=0.31 (cyclohexane/EtOAc=4+1; UV).

¹H-NMR (300 MHz, CDCl₃): δ=8.02 (d, J=8.1 Hz, 2H), 7.56 (d, J=2.1 Hz, 1H), 7.48-7.30 (m, 3H), 5.42-5.23 (m, 1H), 4.35 (q, J=6.4 Hz, 1H), 3.96 (s, 3H), 3.24 (s, 3H), 1.55-1.35 (m, 9H).

¹³C-NMR,APT (76 MHz, CDCl₃): δ=167.3, 165.1, 159.2, 150.5, 145.1, 138.2, 127.6, 126.7, 109.5, 109.4, 79.5, 69.7, 56.6, 55.7, 24.0, 22.0.

HRMS (EI-MS) for C₁₉H₂₃NO₄: calcd=329.1627, found=329.1623, Δm=1.2 ppm.

### Example 63: NG-599

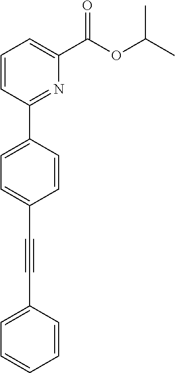 [SMILES: CC(C)OC(=O)c1cccc(-c2ccc(C#Cc3ccccc3)cc2)n1]

The coupling of isopropyl 6-bromopicolinate with 4-(phenylethynyl)phenylboronic acid pinacol ester was performed following the general procedure SC2. An additional crystallization was performed for purification.

Yield=102.9 mg colorless crystals (0.301 mmol, 49%, 2 fractions).

R_{f}=0.43 (cyclohexane/EtOAc=5+1; UV).

¹H-NMR (300 MHz, CDCl₃): δ=8.11 (d, J=8.3 Hz, 2H), 8.02 (dd, J=6.5 Hz, 1.9 Hz, 1H), 7.96-7.84 (m, J=6.8 Hz, 2H), 7.65 (d, J=8.3 Hz, 2H), 7.60-7.50 (m, J=6.2 Hz, 2.8 Hz, 2H), 7.44-7.29 (m, 3H), 5.48-5.23 (m, 1H), 1.45 (d, J=6.2 Hz, 6H).

¹³C-NMR,APT (76 MHz, CDCl₃): δ=164.9, 156.7, 148.9, 138.2, 137.8, 132.2, 131.8, 128.6, 128.5, 127.2, 124.5, 123.5, 123.3, 91.0, 89.4, 69.6, 22.0. 1 carbon signal is missing maybe due to overlap.

HRMS (EI-MS) for C₂₃H₁₉NO₂: calcd=341.1416, found=341.1439, Δm=6.7 ppm.

m.p.=136-143° C.

### Example 64: NG-601

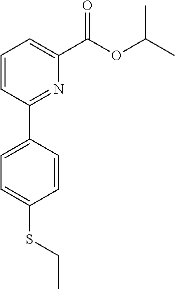 [SMILES: CCSc1ccc(-c2cccc(C(=O)OC(C)C)n2)cc1]

The coupling of isopropyl 6-bromopicolinate with 4-ethylthiobenzeneboronic acid was performed following the general procedure SC1.

Yield=107.2 mg slightly yellowish solid (0.356 mmol, 89%).

R_{f}=0.43 (cyclohexane/EtOAc=5+1; UV).

¹H-NMR (300 MHz, CDCl₃): δ=8.13-7.92 (m, 3H), 7.85 (d, J=3.7 Hz, 2H), 7.40 (d, J=8.3 Hz, 2H), 5.44-5.23 (m, 1H), 3.00 (q, J=7.3 Hz, 2H), 1.55-1.28 (m, 9H).

¹³C-NMR,APT (76 MHz, CDCl₃): δ=165.0, 157.0, 148.8, 138.9, 137.7, 135.9, 128.6, 127.6, 123.2, 122.9, 69.6, 27.3, 22.0, 14.4.

HRMS (EI-MS) for C₁₇H₁₉NO₂S: calcd=301.1136, found=301.1133, Δm=1.0 ppm.

m.p.=93-96° C.

### Example 65: NG-602

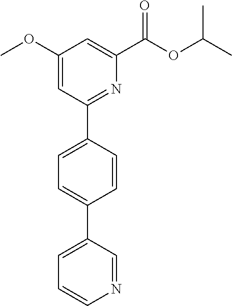 [SMILES: COc1cc(C(=O)OC(C)C)nc(-c2ccc(-c3cccnc3)cc2)c1]

The coupling of isopropyl 6-chloro-4-methoxypicolinate was performed following the general procedure SC2. An additional crystallization was performed for purification.

Yield=32.6 mg colorless solid (0.0936 mmol, 34%).

R_{f}=0.33 (cyclohexane/EtOAc=2+3; UV).

¹H-NMR (300 MHz, CDCl₃): δ=8.91 (s, 1H), 8.61 (d, J=3.4 Hz, 1H), 8.17 (d, J=8.2 Hz, 2H), 7.93 (d, J=7.8 Hz, 1H), 7.69 (d, J=8.2 Hz, 2H), 7.58 (d, J=2.0 Hz, 1H), 7.49-7.31 (m, 2H), 5.46-5.20 (m, J=12.4 Hz, 6.2 Hz, 1H), 3.98 (s, 3H), 1.44 (d, J=6.2 Hz, 6H).

¹³C-NMR,APT (76 MHz, CDCl₃): δ=167.4, 165.0, 158.5, 150.6, 148.8, 148.4, 138.8, 138.5, 136.2, 134.4, 128.0, 127.5, 123.7, 109.6, 109.4, 69.7, 55.7, 22.0.

HRMS (EI-MS) for C₂₁H₂₀N₂O₃: calcd=348.1474, found=348.1463, Δm=3.2 ppm.

m.p.=118-121° C.

### Example 66: NG-605

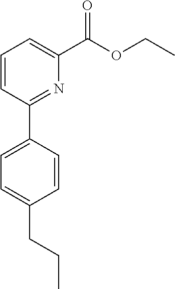 [SMILES: CCCc1ccc(-c2cccc(C(=O)OCC)n2)cc1]

The coupling of ethyl 6-bromopicolinate with 4-propylphenylboronic acid was performed following the general procedure SC1. An additional crystallization was performed for purification.

Yield=218.2 mg colorless solid (0.958 mmol, 68%, 2 fractions).

R_{f}=0.26 (cyclohexane/EtOAc=10+1; UV).

¹H-NMR (300 MHz, CDCl₃): δ=8.10-7.92 (m, 3H), 7.92-7.78 (m, 2H), 7.29 (d, J=8.0 Hz, 2H), 4.48 (q, J=7.1 Hz, 2H), 2.64 (t, J=7.5 Hz, 2H), 1.80-1.58 (m, 2H), 1.46 (t, J=7.1 Hz, 3H), 0.95 (t, J=7.3 Hz, 3H).

¹³C-NMR,APT (76 MHz, CDCl₃): δ=165.7, 157.9, 148.4, 144.4, 137.6, 136.2, 129.1, 127.2, 123.3, 123.1, 61.9, 37.9, 24.6, 14.5, 13.9.

HRMS (EI-MS) for C₁₇H₁₉NO₂: calcd=269.1416, found=269.1412, Δm=1.5 ppm.

m.p.=68-69° C.

### Intermediate NG-607

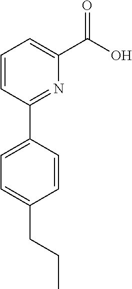 [SMILES: CCCc1ccc(-c2cccc(C(=O)O)n2)cc1]

The saponification of NG-605 was performed following the general procedure SA2.

Yield=189.3 mg colorless solid (0.785 mmol, 98%).

R_{f}=0.59 (CH₂Cl₂/MeOH/HOAc=90+10+1; UV).

¹H-NMR (300 MHz, CDCl₃): δ=8.23-8.08 (m, 1H), 8.06-7.82 (m, 4H), 7.34 (d, J=7.9 Hz, 2H), 2.67 (t, J=7.5 Hz, 2H), 1.86-1.57 (m, 2H), 0.98 (t, J=7.3 Hz, 3H).

¹³C-NMR,APT (76 MHz, CDCl₃): δ=164.3, 156.7, 145.9, 145.4, 139.4, 134.6, 129.4, 127.0, 124.7, 121.7, 37.9, 24.5, 13.9.

HRMS (EI-MS) for C₁₅H₁₅NO₂: calcd=241.1103, found=241.1103, Δm=0.0 ppm.

m.p.=119-121° C.

### Example 67: NG-608

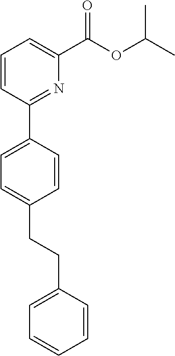 [SMILES: CC(C)OC(=O)c1cccc(-c2ccc(CCc3ccccc3)cc2)n1]

70.4 mg of NG-599 are dissolved in 4.2 mL MeOH to give a 0.05 M solution. Hydrogenation was performed for 3 h using the H-Cube® (10% Pd/C, full H₂ mode, 0.5 mL/min, closed loop).

An column chromatography was performed for purification.

Yield=62.2 mg blue solid (0.180 mmol, 87%).

R_{f}=0.26 (cyclohexane/EtOAc=7+1; UV).

¹H-NMR (300 MHz, CDCl₃): δ=8.17-8.01 (m, 3H), 8.01-7.87 (m, 2H), 7.45-7.32 (m, 4H), 7.32-7.21 (m, 3H), 5.58-5.29 (m, 1H), 3.06 (s, 4H), 1.53 (d, J=6.2 Hz, 6H).

¹³C-NMR,APT (76 MHz, CDCl₃): δ=165.1, 157.6, 148.8, 143.4, 141.7, 137.6, 136.4, 129.1, 128.6, 128.5, 127.3, 126.1, 123.1, 123.1, 69.5, 37.9, 37.8, 22.1.

HRMS (EI-MS) for C₂₃H₂₃NO₂: calcd=345.1729, found=345.1724, Δm=1.4 ppm.

m.p.=88-89° C.

### Example 68: NG-609

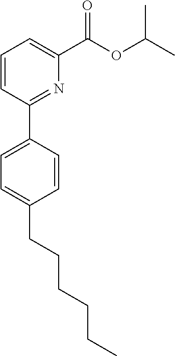 [SMILES: CCCCCCc1ccc(-c2cccc(C(=O)OC(C)C)n2)cc1]

The coupling of isopropyl 6-bromopicolinate with 4-hexylphenylboronic acid was performed following the general procedure SC1. An additional preparative-HPLC (method C) was performed for purification.

Yield=100.4 mg colorless solid (0.308 mmol, 73%).

R_{f}=0.26 (cyclohexane/EtOAc=15+1; UV).

¹H-NMR (300 MHz, CDCl₃): δ=8.13-7.92 (m, 3H), 7.92-7.73 (m, 2H), 7.29 (d, J=8.0 Hz, 2H), 5.52-5.19 (m, 1H), 2.66 (t, J=7.6 Hz, 2H), 1.76-1.54 (m, 2H), 1.44 (d, J=6.2 Hz, 6H), 1.37-1.20 (m, 6H), 0.89 (t, J=6.2 Hz, 3H).

¹³C-NMR,APT (76 MHz, CDCl₃): δ=165.1, 157.8, 148.8, 144.7, 137.6, 136.1, 129.0, 127.2, 123.1, 123.0, 69.5, 35.9, 31.9, 31.5, 29.1, 22.8, 22.1, 14.2.

HRMS (EI-MS) for C₂₁H₂₇NO₂: calcd=325.2042, found=325.2036, Δm=1.8 ppm.

m.p.=34-35° C.

### Example 69: NG-610

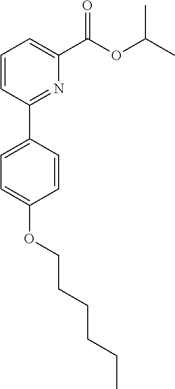 [SMILES: CCCCCCOc1ccc(-c2cccc(C(=O)OC(C)C)n2)cc1]

The coupling of isopropyl 6-bromopicolinate with 4-hexoxyphenylboronic acid was performed following the general procedure SC2.

Yield=100.3 mg colorless solid (0.294 mmol, 69%).

R_{f}=0.28 (cyclohexane/EtOAc=10+1; UV).

¹H-NMR (300 MHz, CDCl₃): δ=8.04 (d, J=8.7 Hz, 2H), 7.98-7.88 (m, 1H), 7.82 (d, J=3.5 Hz, 2H), 6.99 (d, J=8.7 Hz, 2H), 5.49-5.17 (m, 1H), 4.01 (t, J=6.5 Hz, 2H), 1.89-1.74 (m, 2H), 1.57-1.23 (m, 12H), 0.92 (t, J=6.5 Hz, 3H).

¹³C-NMR,APT (76 MHz, CDCl₃): δ=165.1, 160.6, 157.4, 148.6, 137.5, 131.0, 128.6, 122.5, 114.8, 69.4, 68.2, 31.7, 29.3, 25.8, 22.7, 22.0, 14.2. 1 carbon signal is missing maybe due to overlap.

HRMS (EI-MS) for C₂₁H₂₇NO₃: calcd=341.1991, found=341.1988, Δm=0.9 ppm.

m.p.=68-70° C.

### Example 70: NG-613

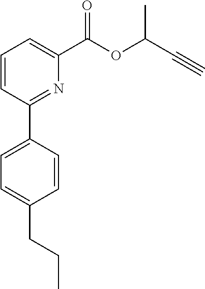 [SMILES: C#CC(C)OC(=O)c1cccc(-c2ccc(CCC)cc2)n1]

The esterification of NG-607 with 3-butyn-2-ol was performed following the general procedure ES1 with the modification that 0.17 eq DMAP were used and the mixture was stirred two times overnight, after which time addition 0.5 eq alcohol, 0.3 eq EDC*HCl, and 0.15 eq DMAP were added and the mixture was then stirred overnight again. For further purification, the product was dissolved in ACN and was washed five times with n-hexane.

Yield=39.1 mg colorless solid (0.33 mmol, 64%).

R_{f}=0.28 (cyclohexane/EtOAc=10+1; UV).

¹H-NMR (300 MHz, CDCl₃): δ=8.11-7.95 (m, 3H), 7.95-7.82 (m, 2H), 7.30 (d, J=8.0 Hz, 2H), 5.92-5.63 (m, 1H), 2.65 (t, J=7.3 Hz, 2H), 2.52 (d, J=2.0 Hz, 1H), 1.78-1.60 (m, 5H), 0.95 (t, J=7.3 Hz, 3H).

¹³C-NMR,APT (76 MHz, CDCl₃): δ=164.4, 157.9, 147.8, 144.5, 137.7, 136.0, 129.1, 127.2, 123.5, 123.4, 82.1, 73.5, 61.6, 38.0, 24.6, 21.4, 13.9.

HRMS (EI-MS) for C₁₉H₁₉NO₂: calcd=293.1416, found=293.1397, Δm=6.5 ppm.

m.p.=90-92° C.

### Example 71: NG-614

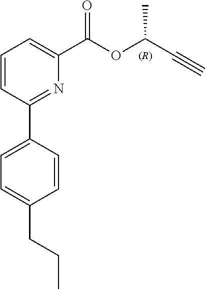

The esterification of NG-607 with (R)-(+)-3-butyn-2-ol was performed following the general procedure ES1 with the modification that the mixture was stirred two times overnight, after which time addition 0.5 eq alcohol, 0.3 eq EDC*HCl, and 0.15 eq DMAP were added and the mixture was then stirred overnight again.

Yield=47.6 mg colorless solid (0.162 mmol, 78%).

R_{f}=0.30 (cyclohexane/EtOAc=10+1; UV).

¹H-NMR (300 MHz, CDCl₃): δ=8.11-7.95 (m, 3H), 7.94-7.81 (m, 2H), 7.29 (d, J=8.1 Hz, 2H), 5.92-5.62 (m, 1H), 2.65 (t, J=7.8 Hz, 2H), 2.52 (d, J=1.5 Hz, 1H), 1.80-1.55 (m, 5H+H₂O peak), 0.96 (t, J=7.3 Hz, 3H).

¹³C-NMR,APT (76 MHz, CDCl₃): δ=164.4, 157.9, 147.8, 144.5, 137.7, 136.0, 129.1, 127.2, 123.5, 123.4, 82.1, 73.5, 61.6, 38.0, 24.6, 21.4, 13.9.

HRMS (EI-MS) for C₁₉H₁₉NO₂: calcd=293.1416, found=293.1404, Δm=4.1 ppm.

m.p.=112-113° C.

[α]²⁰₅₈₉=+8.1 (ρ=0.95; CHCl₃)

### Example 72: NG-615

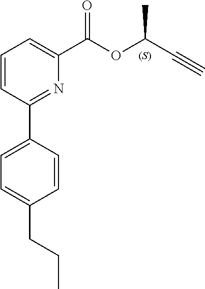

The esterification of NG-607 with (S(−)-3-butyn-2-ol was performed following the general procedure ES1 with the modification that the mixture was stirred two times overnight, after which time addition 0.5 eq alcohol, 0.3 eq EDC*HCl, and 0.15 eq DMAP were added and the mixture was then stirred overnight again. For further purification, the product was dissolved in ACN and was washed five times with n-hexane.

Yield=35.9 mg colorless solid (0.122 mmol, 59%).

R_{f}=0.28 (cyclohexane/EtOAc=10+1; UV).

¹H-NMR (300 MHz, CDCl₃): δ=8.11-7.94 (m, 3H), 7.94-7.81 (m, 2H), 7.29 (d, J=8.1 Hz, 2H), 5.87-5.65 (m, 1H), 2.65 (t, J=7.4 Hz, 2H), 2.52 (d, J=1.9 Hz, 1H), 1.80-1.58 (m, 5H), 0.96 (t, J=7.3 Hz, 3H).

¹³C-NMR,APT (76 MHz, CDCl₃): δ=164.4, 157.9, 147.8, 144.5, 137.7, 136.0, 129.1, 127.2, 123.5, 123.4, 82.1, 73.5, 61.6, 37.9, 24.6, 21.4, 13.9.

HRMS (EI-MS) for C₁₉H₁₉NO₂: calcd=293.1416, found=293.1407, Δm=3.1 ppm.

m.p.=112-113° C.

[α]²⁰₅₈₉=+9.6 (ρ=0.63; CHCl₃)

### Example 73: NG-616

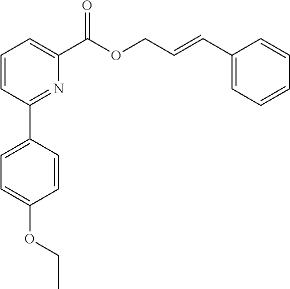 [SMILES: CCOc1ccc(-c2cccc(C(=O)OC/C=C/c3ccccc3)n2)cc1]

The esterification of TSch-42 (see NG-482) with cinnamyl alcohol was performed following the general procedure ES1.

Yield=117.8 mg colorless solid (0.327 mmol, 78%).

R_{f}=0.24 (cyclohexane/EtOAc=7+1; UV).

¹H-NMR (300 MHz, CDCl₃): δ=8.15-7.95 (m, 3H), 7.85 (d, J=4.1 Hz, 2H), 7.44 (d, J=7.0 Hz, 2H), 7.39-7.21 (m, 3H), 7.00 (d, J=8.7 Hz, 2H), 6.81 (d, J=15.9 Hz, 1H), 6.57-6.40 (dt, J=6.4, 18.8 Hz, 1H), 5.09 (d, J=6.2 Hz, 2H), 4.10 (q, J=6.9 Hz, 2H), 1.45 (t, J=7.0 Hz, 3H).

¹³C-NMR,APT (76 MHz, CDCl₃): δ=165.5, 160.4, 157.5, 148.1, 137.6, 136.4, 134.8, 131.0, 128.7, 128.6, 128.2, 126.8, 123.2, 122.9, 122.8, 114.8, 66.4, 63.7, 14.9.

HRMS (EI-MS) for C₂₃H₂₁NO₃: calcd=359.1521, found=359.1512, Δm=2.5 ppm.

m.p.=110° C.

### Example 74: NG-617

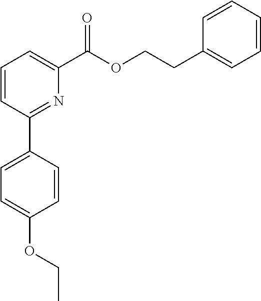 [SMILES: CCOc1ccc(-c2cccc(C(=O)OCCc3ccccc3)n2)cc1]

The esterification of TSch-42 (see NG-482) with 2-phenylethanol was performed following the general procedure ES1. Additional ACN/n-hexane extractions were performed for purification.

Yield=103.8 mg colorless solid (0.299 mmol, 74%).

R_{f}=0.34 (cyclohexane/EtOAc=5+1; UV).

¹H-NMR (300 MHz, CDCl₃): δ=8.04 (d, J=8.8 Hz, 2H), 7.93 (dd, J=5.6 Hz, 3.0 Hz, 1H), 7.88-7.77 (m, 2H), 7.44-7.29 (m, 4H), 7.29-7.20 (m, 1H), 4.62 (t, J=7.1 Hz, 2H), 4.11 (q, J=6.9 Hz, 2H), 3.15 (t, J=7.1 Hz, 2H), 1.45 (t, J=7.0 Hz, 3H).

¹³C-NMR,APT (76 MHz, CDCl₃): δ=165.6, 160.4, 157.4, 148.1, 138.0, 137.6, 129.3, 128.7, 128.6, 126.8, 122.8, 122.7, 114.8, 66.3, 63.7, 35.3, 14.9.

HRMS (EI-MS) for C₂₂H₂₁NO₃: calcd=347.1521, found=347.1517, Δm=1.2 ppm.

m.p.=80-82° C.

### Example 75: NG-618

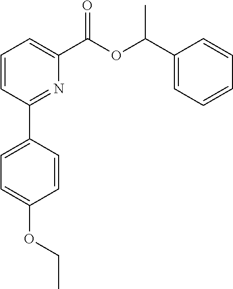 [SMILES: CCOc1ccc(-c2cccc(C(=O)OC(C)c3ccccc3)n2)cc1]

The esterification of TSch-42 (see NG-482) with 1-phenylethanol was performed following the general procedure ES1 with the modification that the mixture was stirred for 91 h, after which time 0.5 eq 1-phenylethanol, 0.3 eq EDC*HCl, and 0.15 eq DMAP were added and the mixture was stirred overnight.

Yield=87.0 mg colorless oil (0.250 mmol, 61%).

R_{f}=0.33 (cyclohexane/EtOAc=5+1; UV).

¹H-NMR (300 MHz, CDCl₃): δ=8.07 (d, J=8.8 Hz, 2H), 7.97 (dd, J=5.9 Hz, 2.6 Hz, 1H), 7.90-7.76 (m, 2H), 7.52 (d, J=7.1 Hz, 2H), 7.45-7.27 (m, 3H), 7.00 (d, J=8.8 Hz, 2H), 6.30-6.14 (m, 1H), 4.10 (q, J=7.0 Hz, 2H), 1.74 (d, J=6.6 Hz, 3H), 1.45 (t, J=7.0 Hz, 3H).

¹³C-NMR,APT (76 MHz, CDCl₃): δ=164.8, 160.4, 157.4, 148.3, 141.8, 137.5, 131.0, 128.7, 128.6, 128.0, 126.3, 127.7, 126.6, 114.8, 73.8, 63.7, 22.6, 14.9.

HRMS (EI-MS) for C₂₂H₂₁NO₃: calcd=347.1521, found=347.1509, Δm=3.5 ppm.

### Example 76: NG-619

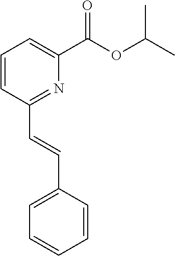 [SMILES: CC(C)OC(=O)c1cccc(/C=C/c2ccccc2)n1]

The coupling of isopropyl 6-bromopicolinate with trans-2-phenylvinylboronic acid was performed following the general procedure SC1.

Yield=210.5 mg slightly yellowish solid (0.787 mmol, 89%).

R_{f}=0.38 (cyclohexane/EtOAc=5+1; UV, KMnO₄).

¹H-NMR (300 MHz, CDCl₃): δ=7.85 (d, J=7.6 Hz, 1H), 7.71 (t, J=7.8 Hz, 1H), 7.64-7.46 (m, 4H), 7.40-7.14 (m, 4H), 5.39-5.15 (m, 1H), 1.36 (d, J=6.3 Hz, 6H).

¹³C-NMR,APT (76 MHz, CDCl₃): δ=164.9, 156.4, 148.7, 137.4, 136.5, 134.1, 128.9, 128.7, 127.9, 127.4, 124.2, 123.2, 69.6, 22.0.

HRMS (EI-MS) for C₁₇H₁₇NO₂: calcd=267.1259, found=267.1248, Δm=4.1 ppm.

m.p.=66-71° C.

### Example 77: NG-620

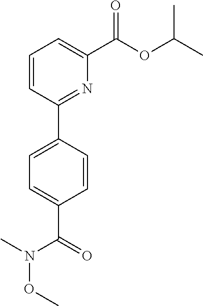 [SMILES: CON(C)C(=O)c1ccc(-c2cccc(C(=O)OC(C)C)n2)cc1]

The coupling of isopropyl 6-bromopicolinate with 4-(N,O-dimethylhydroxylaminocarbonyl)phenylboronic acid was performed following the general procedure SC2.

Yield=99.1 mg yellow oil (0.302 mmol, 69%).

R_{f}=0.54 (cyclohexane/EtOAc=1+2; UV).

¹H NMR (300 MHz, CDCl₃) δ 8.12 (d, J=8.3 Hz, 2H), 8.04 (dd, J=6.2 Hz, 2.4 Hz, 1H), 7.99-7.86 (m, 2H), 7.80 (d, J=8.2 Hz, 2H), 5.45-5.22 (m, 1H), 3.55 (s, 3H), 3.38 (s, 3H), 1.44 (d, J=6.2 Hz, 6H).

¹³C-NMR,APT (76 MHz, CDCl₃): δ=169.6, 164.8, 156.8, 149.0, 140.6, 137.9, 135.0, 128.9, 127.0, 123.8, 123.6, 69.7, 61.3, 33.8, 22.0.

HRMS (EI-MS) for C₁₅H₂₀N₂O₄: calcd=328.1423, found=328.1423, Δm=0 ppm.

### Reference Compound NG-622

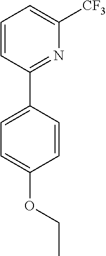 [SMILES: CCOc1ccc(-c2cccc(C(F)(F)F)n2)cc1]

The coupling of 2-bromo-6-(trifluoromethyl)pyridine with 4-ethoxyphenylboronic acid was performed following the general procedure SC1.

Yield=102.4 mg colorless solid (0.383 mmol, 87%).

R_{f}=0.46 (cyclohexane/EtOAc=5+1; UV, KMnO₄).

¹H-NMR (300 MHz, CDCl₃): δ=8.02 (d, J=8.8 Hz, 2H), 7.93-7.78 (m, 2H), 7.53 (dd, J=6.1 Hz, 2.2 Hz, 1H), 6.99 (d, J=8.8 Hz, 2H), 4.10 (q, J=7.0 Hz, 2H), 1.45 (t, J=7.0 Hz, 3H).

¹³C-NMR (126 MHz, CDCl₃): δ=160.7, 157.7, 148.2 (q, J=34.3 Hz), 138.0, 130.4, 128.6, 122.0, 121.8 (q, J=273.7 Hz), 117.8 (q, J=2.8 Hz), 115.0, 63.8, 14.9.

HRMS (EI-MS) for C₁₄H₁₂F₃NO: calcd=267.0871, found=267.0877, Δm=2.2 ppm.

m.p.=100-101° C.

### Example 78: NG-629

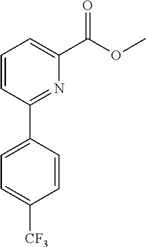 [SMILES: COC(=O)c1cccc(-c2ccc(C(F)(F)F)cc2)n1]

The coupling of methyl-6-bromopicolinate with 4-trifluoromethylphenylboronic acid was performed following the general procedure SC1.

Yield=541.0 mg colorless solid (1.92 mmol, 84%).

R_{f}=0.38 (cyclohexane/EtOAc=3+1; UV, KMnO₄).

¹H-NMR (300 MHz, CDCl₃): δ=8.30-8.06 (m, 3H), 8.04-7.86 (m, 2H), 7.74 (d, J=8.2 Hz, 2H), 4.03 (s, 3H).

¹³C-NMR,APT (76 MHz, CDCl₃): δ=165.9, 156.3, 148.5, 141.9, 138.2, 131.7+131.2 (most probably as part of a quartet), 127.7, 125.9 (q, J=3.7 Hz), 124.3, 124.1, 122.4, 53.1.

HRMS (EI-MS) for C₁₄H₁₀F₃NO₂: calcd=281.0664, found=281.0660, Δm=1.4 ppm.

m.p.=95-96° C.

### Reference Compound NG-631

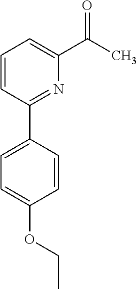 [SMILES: CCOc1ccc(-c2cccc(C(C)=O)n2)cc1]

The coupling of 2-acetyl-6-bromopyridine with 4-ethoxyphenylboronic acid was performed following the general procedure SC1.

Yield=108.0 mg beige solid (0.448 mmol, 85%).

R_{f}=0.55 (cyclohexane/EtOAc=3+1; UV, KMnO₄).

¹H-NMR (300 MHz, CDCl₃): δ=8.05 (d, J=8.7 Hz, 2H), 7.97-7.73 (m, 3H), 7.01 (d, J=8.7 Hz, 2H), 4.11 (q, J=6.9 Hz, 2H), 2.81 (s, 3H), 1.46 (t, J=7.0 Hz, 3H).

¹³C-NMR,APT (76 MHz, CDCl₃): δ=160.4, 156.3, 153.4, 137.6, 131.0, 128.3, 122.7, 119.1, 114.9, 63.7, 25.9, 14.9.

HRMS (EI-MS) for C₁₅H₁₅NO₂: calcd=241.1103, found=241.1095, Δm=3.3 ppm.

m.p.=80-86° C.

### Example 79: NG-633

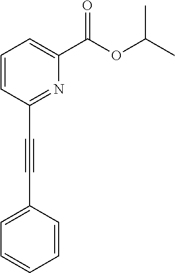 [SMILES: CC(C)OC(=O)c1cccc(C#Cc2ccccc2)n1]

This compound was synthesized using a method described in the literature (J. Org. Chem. 2011, 76, 5320-5334.)

A Schienk tube was dried under vacuum and charged with 151.3 mg (0.620 mmol) isopropyl 6-bromopicolinate, 74.9 μL (6.3 μmol, 1.1 eq) phenylacetylene, 4.4 mg PdCl₂(PPh₃)₂ (6.3 μmol, 1 mol %), 3.5 mg CuI (3.5 mg, 18.4 μmol, 3 mol %), 3.4 mL anhydrous ACN, and 129 μL Et₃N (0.931 mmol, 1.5 eq). The mixture was stirred at 60° C. (oil-bath) overnight, after which time the reaction mixture was cooled to rt and 5 mL H₂O were added. The mixture was extracted with EtOAc (3×5 mL EtOAc) and the combined organic layers were washed with brine (1×5 mL), dried over Na₂SO₄, filtered, and the solvent was removed in vacuo. Final purification via column chromatography yielded the pure product.

Yield=153.7 mg brown solid (0.579 mmol, 93%).

R_{f}=0.52 (cyclohexane/EtOAc=3+1; UV, KMnO₄).

¹H-NMR (300 MHz, CDCl₃): δ=8.02 (d, J=7.7 Hz, 1H), 7.80 (t, J=7.8 Hz, 1H), 7.73-7.50 (m, 3H), 7.47-7.27 (m, 3H), 5.54-5.12 (m, 1H), 1.42 (d, J=6.3 Hz, 6H).

¹³C-NMR,APT (76 MHz, CDCl₃): δ=164.3, 149.3, 143.9, 137.1, 132.2, 130.2, 129.3, 128.5, 123.9, 122.2, 90.4, 88.4, 70.0, 21.9.

HRMS (EI-MS) for C₁₇H₁₅NO₂: calcd=265.1103, found=265.1100, Δm=1.1 ppm.

m.p.=85-86° C.

### Example 80: NG-634

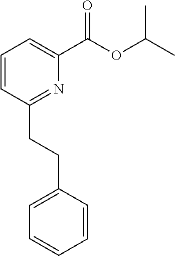 [SMILES: CC(C)OC(=O)c1cccc(CCc2ccccc2)n1]

148.2 mg of NG-619 were dissolved in 11 mL MeOH to give a 0.05 M solution. Hydrogenation was performed for 3.5 h using the H-Cube® (10% Pd/C, full H₂ mode, 0.5 mL/min, closed loop).

Yield=146.0 mg slightly yellow oil (0.542 mmol, 98%).

R_{f}=0.51 (cyclohexane/EtOAc=3+1; UV).

¹H-NMR (300 MHz, CDCl₃): δ=7.92 (d, J=7.6 Hz, 1H), 7.70 (t, J=7.7 Hz, 1H), 7.39-7.09 (m, 6H), 5.45-5.23 (m, 1H), 3.37-3.18 (m, 2H), 3.18-3.00 (m, 2H), 1.44 (d, J=6.2 Hz, 6H).

¹³C-NMR,APT (76 MHz, CDCl₃): δ=164.8, 162.0, 148.2, 141.4, 137.2, 128.7, 128.5, 126.3, 126.1, 122.7, 69.6, 39.9, 35.9, 22.0.

HRMS (EI-MS) for C₁₇H₁₉NO₂: calcd=269.1416, found=269.1402, Δm=5.2 ppm.

### Example 81: NG-635

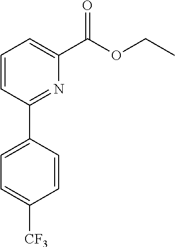 [SMILES: CCOC(=O)c1cccc(-c2ccc(C(F)(F)F)cc2)n1]

The coupling of ethyl 6-bromopicolinate with 4-trifluoromethylphenylboronic acid was performed following the general procedure SC1. Additional ACN/n-hexane extractions were performed for purification.

Yield=84.8 mg colorless solid (0.287 mmol, 65%).

R_{f}=0.51 (cyclohexane/EtOAc=3+1; UV, KMnO₄).

¹H-NMR (500 MHz, CDCl₃): δ=8.19 (d, J=8.2 Hz, 2H), 8.10 (dd, J=5.7 Hz, 3.0 Hz, 1H), 7.97-7.91 (m, 2H), 7.74 (d, J=8.2 Hz, 2H), 4.50 (q, J=7.1 Hz, 2H), 1.47 (t, J=7.1 Hz, 3H).

¹³C-NMR (126 MHz, CDCl₃): δ=165.3, 156.2, 148.8, 142.0, 138.1, 131.5 (q, J=32.9 Hz), 127.7, 125.9 (q, J=3.7 Hz), 124.3 (q, J=272.3 Hz, only 2 signals visible), 124.2, 123.8, 62.1, 14.5.

m.p.=110-112° C.

HRMS (EI-MS) for C₁₅H₁₂F₃NO₂: calcd=295.0820, found=295.0829, Δm=3.1 ppm.

### Example 82: NG-636

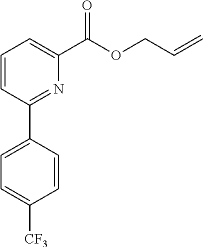 [SMILES: C=CCOC(=O)c1cccc(-c2ccc(C(F)(F)F)cc2)n1]

The procedure is based on the synthesis of a similar substrate (Angew. Chem. Int. Ed. 2014, 53, 10536-10540)

A Schlenk tube was dried under vacuum and was charged with 84.0 mg (0.314 mmol) NG-632, 1.7 mL anhydrous DMF, 42.4 mg (0.505 mmol) NaHCO₃, and 40.8 μL (0.472 mmol) allyl bromide. The mixture was stirred at 50° C. (oil-bath) overnight, until which time TLC indicated all starting material to be consumed. To the mixture were added 5 mL H₂O and the mixture was extracted with CH₂Cl₂ (4×5 mL). The combined organic layers were washed with brine (1×5 mL), dried over Na₂SO₄, filtered and the solvent was removed under reduced pressure. The crude product was purified via column chromatography (cyclohexane/EtOAc=3+1) and 68.8 mg (0.224 mmol, 71%) of NG-636 were isolated as colorless solid. Additional ACN/n-hexane extractions were performed for purification.

R_{f}=0.50 (cyclohexane/EtOAc=3+1; UV, KMnO₄).

¹H-NMR (500 MHz, CDCl₃): δ=8.19 (d, J=8.1 Hz, 2H), 8.14-8.09 (m, 1H), 7.96-7.92 (m, 2H), 7.74 (d, J=8.2 Hz, 2H), 6.10 (ddt, J=17.1 Hz, 10.5 Hz, 5.7 Hz, 1H), 5.49 (ddd, J=17.2 Hz, 2.9 Hz, 1.5 Hz, 1H), 5.34 (ddd, J=10.4 Hz, 2.5 Hz, 1.2 Hz, 1H), 4.94 (dt, J=5.8 Hz, 1.3 Hz, 2H).

¹³C-NMR (126 MHz, CDCl₃): δ=165.0, 156.2, 148.6, 141.9, 138.1, 132.0, 131.4 (q, J=32.5 Hz), 127.7, 125.9 (q, J=3.8 Hz), 124.3 (q, J=272.1 Hz, only 2 signals visible), 124.3, 123.9, 66.6.

HRMS (EI-MS) for C₁₈H₁₂F₃NO₂: calcd=307.0820, found=307.0811, Δm=3.1 ppm.

m.p.=79-80° C.

### Example 83: NG-637

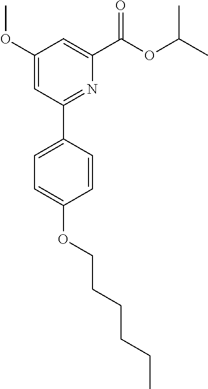 [SMILES: CCCCCCOc1ccc(-c2cc(OC)cc(C(=O)OC(C)C)n2)cc1]

The coupling of isopropyl 6-chloro-4-methoxypicolinate with 4-hexoxyphenylboronic acid was performed following the general procedure SC2. An additional column chromatography and preparative-HPLC (method C) was performed for purification.

Yield=87.9 mg colorless solid (0.237 mmol, 55%).

R_{f}=0.57 (cyclohexane/EtOAc=3+1; UV).

¹H-NMR (300 MHz, CDCl₃): δ=8.00 (d, J=8.8 Hz, 2H), 7.50 (d, J=2.1 Hz, 1H), 7.30 (d, J=2.1 Hz, 1H), 6.97 (d, J=8.8 Hz, 2H), 5.59-5.10 (m, 1H), 4.01 (t, J=6.6 Hz, 2H), 3.95 (s, 3H), 1.91-1.71 (m, 2H), 1.62-1.23 (m, 12H), 0.91 (t, J=6.4 Hz, 3H).

¹³C-NMR,APT (76 MHz, CDCl₃): δ=167.2, 165.2, 160.6, 159.0, 150.3, 131.2, 128.6, 114.7, 108.9, 108.4, 69.6, 68.3, 55.6, 31.7, 29.4, 25.9, 22.7, 22.0, 14.2.

HRMS (EI-MS) for C₂₂H₂₉NO₄: calcd=371.2097, found=371.2088, Δm=2.4 ppm.

m.p.=53-55° C.

### Example 84: NG-639

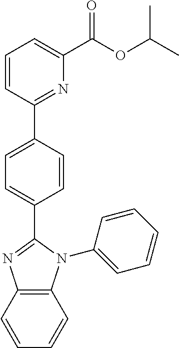 [SMILES: CC(C)OC(=O)c1cccc(-c2ccc(-c3nc4ccccc4n3-c3ccccc3)cc2)n1]

The coupling of isopropyl 6-bromopicolinate with 4-(1-phenyl-1H-benzimidazol-2-yl)phenylboronic acid was performed following the general procedure SC1.

Yield=146.8 mg slightly yellow solid (0.339 mmol, 79%).

R_{f}=0.44 (cyclohexane/EtOAc=1+1; UV, KMnO₄).

¹H-NMR (300 MHz, CDCl₃): δ=8.10-7.96 (m, 3H), 7.96-7.79 (m, 3H), 7.69 (d, J=8.3 Hz, 2H), 7.61-7.42 (m, 3H), 7.42-7.21 (m, 5H), 5.51-5.18 (m, 1H), 1.42 (d, J=6.3 Hz, 6H).

¹³C-NMR,APT (76 MHz, CDCl₃): δ=164.8, 156.7, 152.0, 148.9, 143.2, 139.3, 137.8, 137.5, 137.1, 130.1, 130.0, 128.8, 127.6, 127.2, 123.6, 123.2, 120.1, 110.6, 69.6, 22.0. 1 carbon missing maybe due to overlap.

HRMS (EI-MS) for C₂₈H₂₃N₃O₂: calcd=433.1790, found=433.1794, Δm=0.9 ppm.

m.p.=172-176° C.

### Reference Compound NG-640

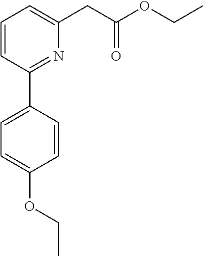 [SMILES: CCOC(=O)Cc1cccc(-c2ccc(OCC)cc2)n1]

The coupling of ethyl 2-(6-bromopyridin-2-yl)acetate with 4-ethoxyphenylboronic acid was performed following the general procedure SC1. An additional crystallization was performed for purification.

Yield=135.2 mg colorless solid (0.474 mmol, 49%).

R_{f}=0.52 (cyclohexane/EtOAc=3+1; UV, KMnO₄).

¹H-NMR (300 MHz, CDCl₃): δ=7.95 (d, J=8.7 Hz, 2H), 7.67 (t, J=7.7 Hz, 1H), 7.55 (d, J=7.8 Hz, 1H), 7.17 (d, J=7.5 Hz, 1H), 6.97 (d, J=8.7 Hz, 2H), 4.21 (q, J=7.1 Hz, 2H), 4.09 (q, J=6.9 Hz, 2H), 3.90 (s, 2H), 1.44 (t, J=7.0 Hz, 3H), 1.28 (t, J=7.1 Hz, 3H).

¹³C-NMR,APT (76 MHz, CDCl₃): δ=171.1, 160.0, 156.9, 154.4, 137.2, 131.9, 128.4, 121.3, 118.1, 114.7, 63.7, 61.0, 44.4, 15.0, 14.4.

HRMS (EI-MS) for C₁₇H₁₉NO₃: calcd=285.1365, found=285.1360, Δm=1.8 ppm.

m.p.=90-91° C.

### Example 85: NG-642

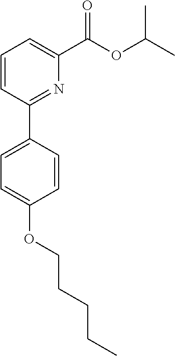 [SMILES: CCCCCOc1ccc(-c2cccc(C(=O)OC(C)C)n2)cc1]

The coupling of isopropyl 6-bromopicolinate with 4-pentyloxyphenylboronic acid was performed following the general procedure SC1.

Yield=130.7 mg slightly yellow solid (0.399 mmol, 96%).

R_{f}=0.61 (cyclohexane/EtOAc=3+1; UV).

¹H-NMR (300 MHz, CDCl₃): δ=8.04 (d, J=8.7 Hz, 2H), 7.98-7.88 (m, 4.0 Hz, 1H), 7.88-7.71 (m, 2H), 6.99 (d, J=8.7 Hz, 2H), 5.56-5.12 (m, 1H), 4.01 (t, J=6.6 Hz, 2H), 1.92-1.71 (m, 2H), 1.44 (d, J=6.2 Hz, 10H), 0.94 (t, J=6.9 Hz, 3H).

¹³C-NMR,APT (76 MHz, CDCl₃): δ=165.1, 160.6, 157.4, 148.6, 137.5, 131.0, 128.6, 122.5, 114.8, 69.4, 68.2, 29.1, 28.3, 22.6, 22.0, 14.2. 1 carbon missing maybe due to overlap.

HRMS (EI-MS) for C₂₀H₂₅NO₃: calcd=327.1834, found=327.1824, Δm=3.1 ppm.

m.p.=63-65° C.

### Example 86: NG-643

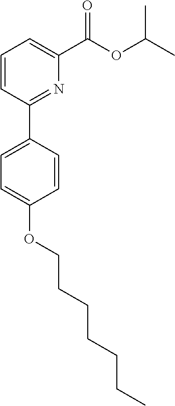 [SMILES: CCCCCCCOc1ccc(-c2cccc(C(=O)OC(C)C)n2)cc1]

The coupling of isopropyl 6-bromopicolinate with heptoxyphenylboronic acid was performed following the general procedure SC1.

Yield=153.6 mg slightly yellow solid (0.432 mmol, 99%).

R_{f}=0.59 (cyclohexane/EtOAc=3+1; UV).

¹H-NMR (300 MHz, CDCl₃): δ=8.04 (d, J=8.7 Hz, 2H), 7.98-7.89 (m, 1H), 7.88-7.75 (m, 2H), 6.99 (d, J=8.7 Hz, 2H), 5.51-5.17 (m, 1H), 4.01 (t, J=6.5 Hz, 2H), 1.91-1.69 (m, 2H), 1.56-1.19 (m, 14H), 0.89 (d, J=6.5 Hz, 3H).

¹³C-NMR,APT (76 MHz, CDCl₃): δ=165.1, 160.6, 157.4, 148.6, 137.5, 131.0, 128.6, 122.5, 114.8, 69.4, 68.3, 31.9, 29.4, 29.2, 26.1, 22.7, 22.1, 14.2. 1 carbon missing maybe due to overlap.

HRMS (EI-MS) for C₂₂H₂₉NO₃: calcd=355.2148, found=355.2164, Δm=4.5 ppm.

m.p.=64-66° C.

### Example 87: NG-647

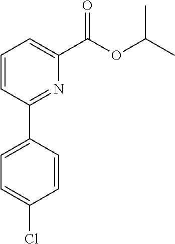 [SMILES: CC(C)OC(=O)c1cccc(-c2ccc(Cl)cc2)n1]

The coupling of isopropyl 6-bromopicolinate with 4-chlorophenylboronic acid was performed following the general procedure SC1. An additional preparative HPLC (method C) was performed for purification.

Yield=74.2 mg colorless solid (0.269 mmol, 65%).

R_{f}=0.44 (cyclohexane/EtOAc=5+1; UV, KMnO₄).

¹H-NMR (300 MHz, CDCl₃): δ=8.12-7.96 (m, 3H), 7.95-7.81 (m, 2H), 7.45 (d, J=8.4 Hz, 2H), 5.34 (hept, J=6.2 Hz, 1H), 1.44 (d, J=6.2 Hz, 6H).

¹³C-NMR,APT (76 MHz, CDCl₃): δ=164.9, 156.4, 148.9, 137.9, 137.1, 135.8, 129.1, 128.6, 123.5, 123.1, 69.6, 22.0.

HRMS (EI-MS) for C₁₅H₁₄ClNO₂: calcd=275.0713, found=275.0722, Δm=3.3 ppm.

m.p.=94-95° C.

### Example 88: NG-648

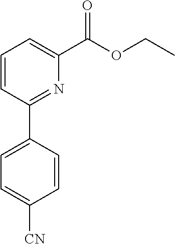 [SMILES: CCOC(=O)c1cccc(-c2ccc(C#N)cc2)n1]

The coupling of ethyl-6-bromopicolinate with 4-cyonophenylboronic acid was performed following the general procedure SC1. An additional preparative HPLC (method C) was performed for purification.

Yield=64.2 mg colorless solid (0.255 mmol, 56%).

R_{f}=0.30 (cyclohexane/EtOAc=3+1; UV).

¹H-NMR (300 MHz, CDCl₃): δ=8.19 (d, J=8.3 Hz, 2H), 8.12 (dd, J=6.6 Hz, 1.9 Hz, 1H), 8.04-7.87 (m, 2H), 7.78 (d, J=8.3 Hz, 2H), 4.50 (q, J=7.1 Hz, 2H), 1.46 (t, J=7.1 Hz, 3H).

¹³C-NMR,APT (76 MHz, CDCl₃): δ=165.2, 155.5, 148.9, 142.7, 138.2, 132.8, 127.9, 124.5, 123.9, 118.8, 113.1, 62.2, 14.4.

HRMS (EI-MS) for C₁₅H₁₂N₂O₂: calcd=252.0899, found=252.0900, Δm=0.4 ppm.

m.p.=108-109° C.

### Example 89: NG-652

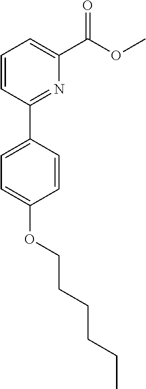 [SMILES: CCCCCCOc1ccc(-c2cccc(C(=O)OC)n2)cc1]

The coupling of methyl 6-bromopicolinate with 4-hexyloxyphenylboronic acid was performed following the general procedure SC1.

Yield=315.2 mg colorless solid (1.01 mmol, 87%).

R_{f}=0.56 (cyclohexane/EtOAc=5+1; UV).

¹H-NMR (300 MHz, CDCl₃): δ=8.13-7.91 (m, 3H), 7.85 (d, J=4.5 Hz, 2H), 6.99 (d, J=8.7 Hz, 2H), 4.01 (t, J=5.5 Hz, 5H), 1.92-1.69 (m, 2H), 1.60-1.19 (m, 6H), 0.92 (t, J=6.6 Hz, 3H).

¹³C-NMR,APT (76 MHz, CDCl₃): δ=166.3, 160.6, 157.6, 148.0, 137.7, 131.0, 128.6, 123.0, 122.7, 114.9, 68.3, 52.9, 31.7, 29.3, 25.8, 22.7, 14.2.

HRMS (EI-MS) for C₁₉H₂₃NO₃: calcd=313.1678, found=313.1672, Δm=1.9 ppm.

m.p.=68-69° C.

### Example 90: NG-658

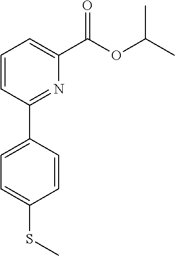 [SMILES: CSc1ccc(-c2cccc(C(=O)OC(C)C)n2)cc1]

The coupling of isopropyl 6-bromopicolinate with 4-(methylthio)benzeneboronic acid was performed following the general procedure SC2. An additional preparative HPLC (method C) was performed for purification.

Yield=83.4 mg colorless solid (0.290 mmol, 69%).

R_{f}=0.57 (cyclohexane/EtOAc=3+1; UV, KMnO₄).

¹H-NMR (300 MHz, CDCl₃): δ=8.14-7.92 (m, 3H), 7.92-7.78 (m, 2H), 7.34 (d, J=8.4 Hz, 2H), 5.33 (hept, J=6.3 Hz, 1H), 2.53 (s, 3H), 1.43 (d, J=6.2 Hz, 6H).

¹³C-NMR,APT (76 MHz, CDCl₃): δ=165.0, 157.0, 148.8, 140.6, 137.7, 135.3, 127.6, 126.5, 123.1, 122.8, 69.5, 22.0, 15.6.

HRMS (EI-MS) for C₁₆H₁₇NO₂S: calcd=287.0980, found=handed in, Δm=xx ppm.

m.p.=60-61° C.

### Example 91: NG-662

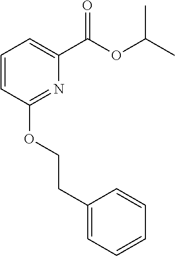 [SMILES: CC(C)OC(=O)c1cccc(OCCc2ccccc2)n1]

The procedure is based on the literature and was modified (Angew. Chem. Int. Ed. 2012, 51, 9071-9074).

A Schienk tube was charged with 101.5 mg (0.416 mmol) isopropyl 6-bromopicolinate, 23.2 mg (0.0418 mmol, 10 mol %) Josiphos SL-J009-1, 19.2 mg (0.0186 mmol, 4.5 mol %) Pd₂(dba)-CHCl₃, and 27.1 mg (0.0832 mmol, 20 mol %->mistake: 2 eq should be used) Cs₂CO₃. The mixture was degassed via three cycles of vacuum/argon, after which time 2 mL anhydrous toluene, and 49.8 μL (0.416 mmol, 1.0 eq) 2-phenylethanol were added. The mixture was stirred at 80° C. (oil-bath) for 7 d, after which time the mixture was poured into 5 mL H₂O and 5 mL EtOAc and the aqueous layer was extracted with EtOAc (2×5 mL). The combined organic layers were washed with brine (1×5 mL), dried over Na₂SO₄, filtered, and the solvent was removed under reduced pressure. The crude product was purified via column chromatography (cyclohexane/EtOAc=3+1) and an additional preparative-HPLC (method C) was performed for purification. 19.4 mg (0.0680 μmol, 16%) of NG-662 were isolated as clear, yellow oil.

R_{f}=0.71 (cyclohexane/EtOAc=3+1; UV, KMnO₄).

¹H-NMR (300 MHz, CDCl₃): δ=7.74-7.59 (m, 2H), 7.42-7.14 (m, 5H), 6.89 (dd, J=6.0 Hz, 3.0 Hz, 1H), 5.28 (hept, J=6.2 Hz, 1H), 4.63 (t, J=7.1 Hz, 2H), 3.11 (t, J=7.1 Hz, 2H), 1.40 (d, J=6.2 Hz, 6H).

¹³C-NMR,APT (76 MHz, CDCl₃): δ=164.8, 163.6, 146.2, 139.0, 138.6, 129.3, 128.5, 126.5, 118.5, 115.3, 69.2, 66.9, 35.5, 22.0.

HRMS (EI-MS) for C₁₇H₁₉NO₃: calcd=285.1365, found=285.1364, Δm=0.4 ppm.

### Example 92: NG-666

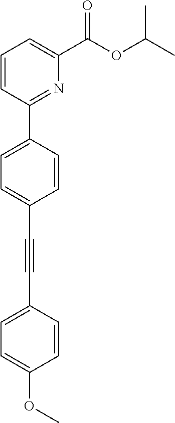 [SMILES: COc1ccc(C#Cc2ccc(-c3cccc(C(=O)OC(C)C)n3)cc2)cc1]

The coupling of isopropyl 6-bromopicolinate with 4-(4-methoxyphenylethynyl)benzeneboronic acid pinacol ester was performed following the general procedure SC1.

Yield=45.2 mg colorless solid (0.127 mmol, 30%).

R_{f}=0.49 (cyclohexane/EtOAc=3+1; UV, KMnO₄).

¹H-NMR (300 MHz, CDCl₃): δ=8.09 (d, J=8.3 Hz, 2H), 8.01 (dd, J=6.6, 1.8 Hz, 1H), 7.88 (q, J=7.6 Hz, 2H), 7.62 (d, J=8.3 Hz, 2H), 7.50 (d, J=8.7 Hz, 2H), 6.89 (d, J=8.7 Hz, 2H), 5.34 (hept, J=6.2 Hz, 1H), 3.83 (s, 3H), 1.44 (d, J=6.2 Hz, 6H).

¹³C-NMR,APT (76 MHz, CDCl₃): δ=164.9, 159.9, 156.8, 148.9, 137.9, 137.8, 133.3, 132.0, 127.1, 124.9, 123.4, 123.2, 115.4, 114.2, 91.1, 88.2, 69.6, 55.5, 22.0.

HRMS (EI-MS) for C₂₄H₂₁NO₃: calcd=371.1521, found=handed in, Δm=xx ppm.

m.p.=152-155° C.

### Example 93: STS-9

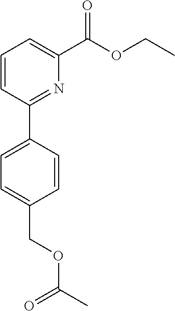 [SMILES: CCOC(=O)c1cccc(-c2ccc(COC(C)=O)cc2)n1]

The coupling of ethyl 6-bromopicolinate with 4-(acetoxymethyl)benzeneboronic acid was performed following the general procedure SC1.

Yield=93.0 mg slightly blue oil (0.311 mmol, 70%).

R_{f}=26 (cyclohexane/EtOAc=4+1; UV).

¹H-NMR (300 MHz, CDCl₃): δ=8.15-7.99 (m, 3H), 7.89 (d, J=4.2 Hz, 2H), 7.47 (d, J=8.1 Hz, 2H), 5.16 (s, 2H), 4.49 (q, J=7.1 Hz, 2H), 2.12 (s, 3H), 1.45 (t, J=7.1 Hz, 3H).

¹³C-NMR,APT (76 MHz, CDCl₃): δ=171.0, 165.6, 157.2, 148.6, 138.6, 137.8, 137.4, 128.7, 127.5, 123.6, 66.0, 62.0, 21.1, 14.4.

HRMS (EI-MS) for C₁₇H₁₇NO₄: calcd=299.1158, found=299.1141, Δm=5.7 ppm.

### Example 94: STS-15

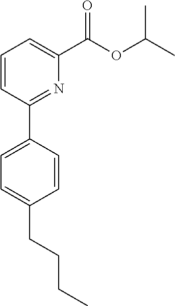 [SMILES: CCCCc1ccc(-c2cccc(C(=O)OC(C)C)n2)cc1]

The coupling of isopropyl 6-bromopicolinate with 4-butylphenylboronic acid was performed following the general procedure SC1.

Yield=98.9 mg slightly yellow solid (0.333 mmol, 80%).

R_{f}=0.29 (cyclohexane/EtOAc=10+1; UV).

¹H NMR (300 MHz, CDCl₃) δ 8.10-7.91 (m, 3H), 7.91-7.79 (m, 2H), 7.29 (d, J=8.1 Hz, 2H), 5.45-5.22 (m, 1H), 2.67 (t, J=7.6 Hz, 2H), 1.62 (dd, J=15.8 Hz, 8.2 Hz, 2H), 1.50-1.26 (m, 8H), 0.94 (t, J=7.3 Hz, 3H).

¹³C-NMR,APT (76 MHz, CDCl₃): δ=165.1, 157.8, 148.7, 144.6, 137.6, 136.1, 129.0, 127.2, 123.1, 123.0, 69.5, 35.6, 33.6, 22.4, 22.0, 14.1.

HRMS (EI-MS) for C₁₉H₂₃NO₂: calcd=297.1729, found=297.1730, Δm=0.3 ppm.

m.p.=51-55° C.

### Example 95: STS-18

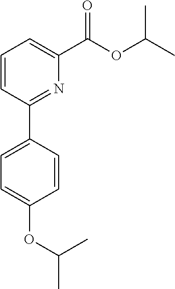 [SMILES: CC(C)OC(=O)c1cccc(-c2ccc(OC(C)C)cc2)n1]

The coupling of isopropyl 6-bromopicolinate with 4-isopropylphenylboronic acid was performed following the general procedure SC1. An additional preparative-HPLC (method B) was performed for purification.

Yield=20.2 mg slightly yellow oil (0.0675 mmol, 16%).

R_{f}=0.29 (cyclohexane/EtOAc=7+1; UV).

¹H-NMR (300 MHz, CDCl₃): δ=8.03 (d, J=8.7 Hz, 2H), 7.93 (dd, J=8.5 Hz, 4.1 Hz, 1H), 7.81 (d, J=3.9 Hz, 2H), 6.97 (d, J=8.7 Hz, 2H), 5.55-5.14 (m, J=12.4 Hz, 6.2 Hz, 1H), 4.82-4.43 (m, J=12.0 Hz, 6.0 Hz, 1H), 1.40 (dd, J=20.4 Hz, 6.1 Hz, 12H).

¹³C-NMR,APT (76 MHz, CDCl₃): δ=165.1, 159.3, 157.4, 148.6, 137.5, 131.0, 128.6, 122.5, 116.1, 70.1, 69.4, 22.1, 22.0.

HRMS (EI-MS) for C₁₈H₂₁NO₃: calcd=299.1521, found=299.1517, Δm=1.3 ppm.

### Example 96: STS-19

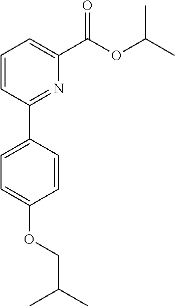 [SMILES: CC(C)COc1ccc(-c2cccc(C(=O)OC(C)C)n2)cc1]

The coupling of isopropyl 6-bromopicolinate with 4-Isobutoxyphenylboronic acid was performed following the general procedure SC1. An additional crystallization was performed for purification.

Yield=64.1 mg colorless needles (0.205 mmol, 50%).

R_{f}=0.25 (cyclohexane/EtOAc=7+1; UV).

¹H-NMR (300 MHz, CDCl₃): δ=8.04 (d, J=8.7 Hz, 2H), 7.98-7.89 (m, 1H), 7.89-7.75 (m, 2H), 6.99 (d, J=8.8 Hz, 2H), 5.45-5.22 (m, 1H), 3.78 (d, J=6.5 Hz, 2H), 2.23-2.02 (m, 1H), 1.43 (d, J=6.2 Hz, 6H), 1.04 (d, J=6.7 Hz, 6H).

¹³C-NMR,APT (76 MHz, CDCl₃): δ=165.1, 160.7, 157.4, 148.6, 137.5, 131.0, 128.6, 122.5, 114.9, 69.4, 28.4, 22.0, 19.4.

HRMS (EI-MS) for C₁₉H₂₃NO₃: calcd=301.1136, found=313.1673, Δm=1.6 ppm.

m.p.=103-107° C.

### Example 97: STS-25

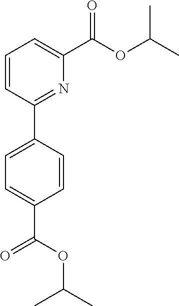 [SMILES: CC(C)OC(=O)c1ccc(-c2cccc(C(=O)OC(C)C)n2)cc1]

The coupling of isopropyl 6-bromopicolinate with 4-(isopropoxycarbonyl)phenylboronic acid was performed following the general procedure SC1. An additional column chromatography was performed for purification.

Yield=91.0 mg colorless oil (0.278 mmol, 60%).

R_{f}=0.54 (cyclohexane/EtOAc=3+1; UV, KMnO₄).

¹H-NMR (300 MHz, CDCl₃): δ=8.26-7.99 (m, 5H), 7.99-7.84 (m, 2H), 5.44-5.20 (m, 2H), 1.42 (2×d).

¹³C-NMR,APT (76 MHz, CDCl₃): δ=166.0, 164.8, 156.5, 149.0, 142.5, 137.9, 131.7, 130.1, 127.2, 123.9, 123.7, 69.7, 68.7, 22.1, 22.0.

HRMS (EI-MS) for C₁₆H₁₆FNO₃: calcd=289.1114, found=327.1459, Δm=3.7 ppm.

### Examples 98 to 100

General Procedures

Suzuki Coupling Procedure A

A 20 mL Schlenk tube with magnetic stirring bar was charged with 1.0 eq boronic acid, 2.0 eq potassium carbonate and 5.0 mol-% Pd(PPh₃)₄ in a 10:1 (v/v) mixture of toluene/H₂O. The reaction mixture was degassed by evacuation and purging with Ar (repeated three times) and subsequently 1.0 eq ethyl 5-bromo-2-furoate was added via Eppendorf® pipette in an Ar counterstream. The reaction vessel was placed in an oil bath and stirred at 80° C. After 24 h reaction time 3.0 mol-% Pd(PPh₃)₄ were added additionally and the temperature was set to 90° C. and 100° C. after 30 h and 46 h, respectively. After 52 h the reaction mixture was cooled to RT and filtered through a pad of Celite®. The filter cake was washed with EtOAc (3×6 mL), the volatiles were removed under reduced pressure and the crude residue was died in oil-pump vacuum. Pure product was obtained via flash column chromatography (SiO₂, cyclohexane/EtOAc 15:1).

Suzuki Coupling Procedure B

A predried 20 mL Schlenk tube with magnetic stirring bar was charged with 5 mol-% PdCl₂(dppf), 1.0 eq aryl halide, 1.0 eq boronic acid and 2.1 eq cesium fluoride in anhydrous DME. The reaction mixture was degassed by evacuation and purging with Ar (repeated three times) and placed in an oil bath at 80° C. After stirring for 24-96 h (reaction control via TLC and GC/MS) the reaction mixture was cooled to RT and filtered through a pad of Celite®. The filter cake was washed with an appropriate amount of EtOAc, the volatiles were removed under reduced pressure and the crude residue was died in oil-pump vacuum. Pure product was obtained via flash column chromatography (SiO₂, cyclohexane/EtOAc).

Suzuki Coupling Procedure C

A 15-50 mL Schlenk-tube with magnetic stirring bar was evacuated and purged with Ar (repeated three times). The Schlenk-tube was subsequently charged with 5 mol-% Pd(OAc)₂, 10 mol-% SPhos, 1.2 eq boronic acid, 1.0 eq aryl halide (if solid) and 5.0 mL 1,4-dioxane. At this point 1.0 eq aryl halide (if liquid) and 1.0 mL of a 3.4 M K₃PO₄ solution (degassed) was added via Eppendorf® pipette in an Ar counterstream. The reaction mixture was degassed by evacuation and purging with Ar (repeated three times) and placed in an oil bath at 60° C. After stirring for 18-22 h (reaction control via TLC and GC/MS) the reaction mixture was cooled to RT, the phases separated and the organic phase filtered through a pad of Celite®. The filter cake was washed with an appropriate amount of EtOAc, the volatiles were removed under reduced pressure and the crude residue was died in oil-pump vacuum. Pure product was obtained via flash column chromatography (SiO₂, cyclohexane/EtOAc).

### Example 98: Ethyl 2-(4-(ethylthio)phenyl)thiazole-4-carboxylate (CLF-3-205)

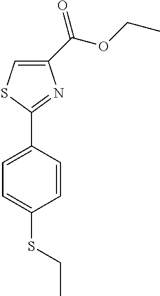 [SMILES: CCOC(=O)c1csc(-c2ccc(SCC)cc2)n1]

4-(Ethylthio)benzeneboronic acid (92.5 mg, 0.508 mmol, 1.2 eq), ethyl 2-bromothiazole-4-carboxylate (100.0 mg, 0.424 mmol, 1.0 eq) and potassium phosphate (726.5 mg, 3.423 mmol, 8.1 eq) in 6.0 mL 1,4-dioxane/H₂O 5:1 (v/v) were reacted in presence of Pd(OAc)₂ (4.8 mg, 0.021 mmol, 5 mol-%) and SPhos (17.4 mg, 0.042 mmol, 10 mol-%) for 18 h at 60° C. according to general procedure C. The crude product was purified via flash column chromatography (16 g SiO₂, cyclohexane/EtOAc 10:1 (v/v), column size 13×2 cm) to obtain the pure product as pale yellow solid.

Yield: 69.9 mg (0.238 mmol, 56%), pale yellow solid.

C₁₄H₁₅NO₂S₂[293.40 g/mol].

R_{f}=0.38 (cyclohexane/EtOAc=5:1 (v/v); staining: KMnO₄).

¹H NMR (300 MHz, CDCl₃): δ=8.12 (s, 1H, Ar—H), 7.91 (d, J=8.4 Hz, 2H, Ar—H), 7.33 (d, J=8.3 Hz, 2H, Ar—H), 4.44 (q, J=7.1 Hz, 2H, CH₂), 3.01 (q, J=7.3 Hz, 2H, CH₂′), 1.52-1.26 (m, 6H, 2×CH₃).

¹³C NMR (75 MHz, CDCl₃): δ=168.5, 161.6, 148.2, 141.0, 130.0, 127.9, 127.4, 126.8, 61.6, 26.9, 14.5, 14.2.

HRMS (DI-EI): Calcd. for C₁₄H₁₅NO₂S₂: 293.0544; found: 293.0546.

### Example 99: Ethyl 2-(4-(methylthio)phenyl)thiazole-4-carboxylate (CLF-3-206)

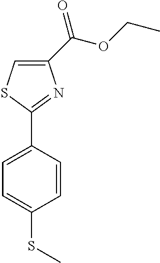 [SMILES: CCOC(=O)c1csc(-c2ccc(SC)cc2)n1]

4-(Methylthio)benzeneboronic acid (85.4 mg, 0.508 mmol, 1.2 eq), ethyl 2-bromothiazole-4-carboxylate (100.0 mg, 0.424 mmol, 1.0 eq) and potassium phosphate (726.5 mg, 3.423 mmol, 8.1 eq) in 6.0 mL 1,4-dioxane/H₂O 5:1 (v/v) were reacted in presence of Pd(OAc)₂ (4.8 mg, 0.021 mmol, 5 mol-%) and SPhos (17.4 mg, 0.042 mmol, 10 mol-%) for 18 h at 60° C. according to general procedure C. The crude product was purified via flash column chromatography (18 g SiO₂, cyclohexane/EtOAc 10:1 (v/v), column size 13×2 cm) to obtain the pure product as pale yellow solid.

Yield: 59.0 mg (0.211 mmol, 50%), pale yellow solid.

C₁₃H₁₃NO₂S₂[279.37 g/mol].

R_{f}=0.34 (cyclohexane/EtOAc=5:1 (v/v); staining: KMnO₄).

¹H NMR (300 MHz, CDCl₃): δ=8.12 (s, 1H, Ar—H), 7.92 (d, J=8.4 Hz, 2H, Ar—H), 7.28 (d, J=8.5 Hz, 2H, Ar—H), 4.44 (q, J=7.1 Hz, 2H, CH₂), 2.52 (s, 3H, CH₃), 1.42 (t, J=7.1 Hz, 3H, CH₃′).

¹³C NMR (75 MHz, CDCl₃): δ=168.6, 161.6, 148.2, 145.5, 129.5, 127.4, 126.7, 126.1, 61.6, 15.3, 14.5.

HRMS (DI-EI): Calcd. for C₁₃H₁₃NO₂S₂: 279.0388; found: 279.0394.

### Example 100: Ethyl 2-(4-isopropylthio)phenyl)thiazole-4-carboxylate (CLF-3-213)

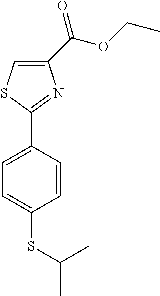 [SMILES: CCOC(=O)c1csc(-c2ccc(SC(C)C)cc2)n1]

4-(Isopropylthio)benzeneboronic acid (99.7 mg, 0.508 mmol, 1.2 eq), ethyl 2-bromothiazole-4-carboxylate (100.0 mg, 0.424 mmol, 1.0 eq) and potassium phosphate (726.5 mg, 3.423 mmol, 8.1 eq) in 6.0 mL 1,4-dioxane/H₂O 5:1 (v/v) were reacted in presence of Pd(OAc)₂ (4.8 mg, 0.021 mmol, 5 mol-%) and SPhos (17.4 mg, 0.042 mmol, 10 mol-%) for 20 h at 60° C. according to general procedure C. The crude product was purified via flash column chromatography (22 g SiO₂, cyclohexane/EtOAc 15:1 (v/v), column size 14×2 cm) to obtain the pure product as pale yellow wax.

Yield: 77.9 mg (0.253 mmol, 60%), pale yellow wax.

C₁₅H₁₇NO₂S₂[307.43 g/mol].

R_{f}=0.15 (cyclohexane/EtOAc=15:1 (v/v); staining: CAM).

¹H NMR (300 MHz, CDCl₃): δ=8.13 (s, 1H, Ar—H), 7.91 (d, J=8.3 Hz, 2H, Ar—H), 7.39 (d, J=8.3 Hz, 2H, Ar—H), 4.44 (q, J=7.1 Hz, 2H, CH₂), 3.60-3.38 (m, 1H, CH̅(CH₃)₂), 1.42 (t, J=7.1 Hz, 3H, CH₂CH₃), 1.34 (d, J=6.6 Hz, 6H, CH(CH₃)₂).

¹³C NMR (75 MHz, CDCl₃): δ=168.5, 161.6, 148.2, 140.0, 130.7, 130.5, 127.4, 127.0, 61.6, 37.6, 23.1, 14.5.

HRMS (DI-EI): Calcd. for C₁₅H₁₇NO₂S₂: 307.0701; found: 307.0710.

### Examples 101 to 181

General Procedure A: Suzuki Coupling

In an inert Schlenk flask equipped with magnetic stirring bar heterocyclic bromide (1.0 eq), boronic acid (0.9 to 1.5 eq) and K₂CO₃ (2.0 eq) were dissolved in degassed toluene abs. (0.1 M). Pd[PPh₃]₄ (3 mol %) was added and the reaction mixture was stirred at 80° C. The reaction was monitored via TLC. When full conversion was observed, the reaction mixture was cooled down to RT and filtered through a pad of Celite. The solvent was removed under reduced pressure and the crude product was purified via column chromatography or preparative HPLC, respectively.

General Procedure B: Fischer-Esterification

In a round-bottom flask heterocyclic acid (1.0 eq.) was dissolved in the corresponding alcohol (0.1-0.2 M) and H₂SO₄ (3.0 eq.) was added. The reaction mixture was equipped with an air condenser and stirred at reflux until full conversion was detected via TLC. The reaction mixture was cooled to RT and the solvent was removed under reduced pressure. The residue was taken up in NaHCO₃ sat and extracted with DCM (3×15 mL). The combined organic phase was dried over Na₂SO₄, filtered and the solvent was removed under reduced pressure. The crude product was used in the next step without further purification.

General Procedure C: DCC-Mediated Esterification

In an inert 10 mL Schienk flask 2-(4-ethoxyphenyl)thiazole acid (1.0 eq) was dissolved in DCM abs. (0.1 M). DCC (1.5 eq.) and DMAP (0.2 eq.) were added successively and the reaction mixture was cooled to 0° C. using an ice bath. The corresponding alcohol (1.5 eq.) was added and the cloudy reaction mixture was stirred at RT until full conversion was observed via TLC. The reaction mixture was filtered through a pad of Celite and the solvent was removed under reduced pressure. The crude product was purified via column chromatography or preparative HPLC, respectively.

General Procedure D: Ullman-Type Coupling

In an inert 10 mL Schlenk flask tert-butyl 5-chloro-2-(4-ethoxyphenyl)thiazole-4-carboxylate (1.0 eq), CuI (0.1 eq) and K₂CO₃ (2.0 eq) were dissolved in anhydrous DMF (0.1 M). The corresponding amine (2.1 eq) was added and the reaction mixture was stirred at 100° C. until full conversion was observed via TLC. The reaction mixture was quenched via the addition of NH4Cl and extracted with EA (3×). The combined organic phase was dried over Na₂SO₄, filtered and the solvent was removed under reduced pressure. The crude product was purified via column chromatography.

Preparative HPLC was performed on a Thermo Scientific UltiMate 3000 system. Detection was accomplished with a Dionex UltiMate DAD. The separations were carried out on a Macherey-Nagel 125/21 Nucleodur 100-5C18EC (125×21 mm, 5.0 μm) column. As eluents MeCN and water with 0.05% HCOOH were used. Following methods were used:

Method A 0.0 min-8.0 min linear increase 40 to 100% MeCN, 8.0-10.0 min 100% MeCN isocratic, 10.0-12.0 min linear decrease 100 to 40% MeCN, 12.0-14.0 min 40% MeCN isocratic, 12 mL/min, 30° C.

Method B 0.0 min-8.0 min linear increase 60 to 100% MeCN, 8.0-10.0 min 100% MeCN isocratic, 10.0-12.0 min linear decrease 100 to 60% MeCN, 12.0-14.0 min 40% MeCN isocratic, 12 mL/min, 30° C.

Method C 0.0 min-6.0 min linear increase 40 to 80% MeCN, 6.0-7.0 min linear increase 80 to 100% MeCN, 7.0-9.0 min 100% MeCN isocratic, 9.0-12.0 min linear decrease 100 to 40% MeCN, 12.0-14.0 min 40% MeCN isocratic, 12 mL/min, 30° C.

Method D 0.0 min-14.0 min 50% MeCN isocratic, 14.0-16.0 min 50 to 100% MeCN 100% linear increase, 16.0-18.0 min 100% MeCN isocratic, 18.0-20.0 min linear decrease 100 to 50% MeCN, 20.0-22.0 min 50% MeCN isocratic, 12 mL/min, 30° C.

Method E 0.0 min-17.0 min linear increase 2 to 100% MeCN, 17.0-19.0 min 100% MeCN isocratic, 19.0-22.0 min linear decrease 100 to 2% MeCN, 22.0-24.0 min 2% MeCN isocratic, 12 mL/min, 30° C.

Method F 0.0 min-2.0 min linear increase 3 to 10% MeCN, 2.0 to 4.0 min 10% MeCN isocratic, 4.0-16.0 min linear increase to 95% MeCN, 17.0-21.0 min 100% MeCN isocratic, 21.0-23.0 min 3% MeCN isocratic, 14 mL/min, 30° C.

Method G 0.0 min-3.0 min 80% MeCN isocratic, 3.0 to 6.0 min linear increase to 100% MeCN, 6.0-8.0 min 100% MeCN isocratic, 8.0-10.0 min linear decrease to 80% MeCN, 10.0-14.0 min 80% MeCN isocratic, 12 mL/min, 30° C.

### Example 101: Ethyl 2-(4-ethoxyphenyl)thiazole-4-carboxylate (AM-50)

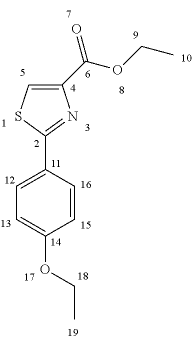 [SMILES: CCOC(=O)c1csc(-c2ccc(OCC)cc2)n1]

According to General Procedure A, ethyl 2-bromothiazole-4-carboxylate (1.0 eq, 805 μmol, 200 mg) was coupled with 4-ethoxyphenylboronic acid (0.9 eq, 724 μmol, 120 mg). The crude product was purified via column chromatography (50 mL SiO₂, eluent CH/EA 8:1).

Yield: 143 mg (516 μmol, 73%) light yellow solid

C₁₄H₁₇NO₃S [277.34]

m.p.: 90° C.

R_{f}: 0.36 (CH/EA 4:1)

HR-MS [EI, M⁺]: calcd. 277.0773, found 277.0771.

¹H-NMR (300 MHz, CDCl₃): δ (ppm) 8.0 (s, 1H, H5), 7.93 (d, J=8.8 Hz, 2H, H12/16), 6.94 (d, J=8.7 Hz, 2H, H13/15), 4.44 (q, J=7.1 Hz, 2H, H9), 4.08 (q, J=6.9 Hz, 2H, H18), 1.43 (m, 6H, H10/19).

¹³C-NMR (75.5 MHz, CDCl₃): δ (ppm) 169.0 (C_{q}, C2), 161.7 (C_{q}, C6), 161.2 (C_{q}, C14), 148.0 (C_{q}, C4), 128.7 (2 CH, C12/16), 126.3 (CH, C5), 125.7 (C_{q}, C11), 114.9 (2 CH, C13/15), 63.8 (CH₂, C9), 61.6 (CH₂, C18), 14.9 (CH₃, C10), 14.5 (CH₃, C19).

### Example 102: Ethyl 2-(4-methoxyphenyl)thiazole-4-carboxylate (AM-1-71)

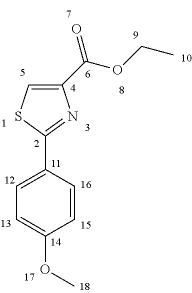 [SMILES: CCOC(=O)c1csc(-c2ccc(OC)cc2)n1]

According to General Procedure A, ethyl 2-bromothiazole-4-carboxylate (1.0 eq, 805 μmol, 200 mg) was coupled with 4-methoxyphenylboronic acid (0.9 eq, 724 μmol, 110 mg). The crude product was purified via column chromatography (50 mL SiO₂, eluent CH/EA 5:1).

Yield: 127 mg (516 μmol, 66%) light yellow solid

C₁₃H₁₃NO₃S [263.31]

m.p.: 100° C.

R_{f}: 0.32 (CH/EA 4:1)

¹H-NMR (300 MHz, CDCl₃): δ (ppm) 8.08 (s, 1H, H5), 7.95 (d, J=8.8 Hz, 2H, H12/16), 6.95 (d, J=8.8 Hz, 2H, H13/15), 4.44 (q, J=7.1 Hz, 2H, H9), 3.86 (s, 3H, H18), 1.42 (t, J=7.1 Hz, 3H, H10)

¹³C-NMR (75.5 MHz, CDCl₃): δ (ppm) 168.9 (C_{q}, C2), 161.8 (C_{q}, C6), 161.7 (C_{q}, C14), 148.0 (C_{q}, C4), 128.7 (2C, CH, C12/16), 126.4 (CH, C6), 125.9 (C_{q}, C11), 114.4 (2C, CH, C13/15), 61.6 (CH₂, C9), 55.6 (CH₃, C18), 14.5 (CH₃, C10).

### Example 103: Ethyl 2-(4-(dimethylamino)phenyl)thiazole-4-carboxylate (AM-1-74)

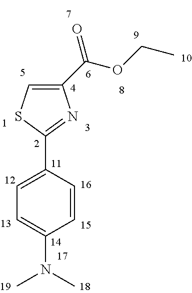 [SMILES: CCOC(=O)c1csc(-c2ccc(N(C)C)cc2)n1]

According to General Procedure A, ethyl 2-bromothiazole-4-carboxylate (1.0 eq, 805 μmol, 200 mg) was coupled with 4-dimethylaminophenylboronic acid (0.9 eq, 724 μmol, 120 mg). The crude product was purified via column chromatography (50 mL SiO₂, eluent CH/EA 4:1) and preparative HPLC (method A).

Yield: 72 mg (516 μmol, 36%) colorless crystals

C14H16N2O2S [276.35]

m.p.: 126° C.

R_{f}: 0.30 (CH/EA 4:1)

HR-MS [EI, M⁺]: calcd. 276.0932, found 276.0929.

¹H-NMR (300 MHz, CDCl₃): δ (ppm) 8.00 (s, 1H, H5), 7.87 (d, J=8.7 Hz, 2H, H12/16), 6.71 (d, J=8.6 Hz, 2H, H13/15), 4.43 (q, J=7.0 Hz, 2H, H9), 3.03 (s, 6H, H18/19), 1.42 (t, J=7.1 Hz, 3H, H10).

¹³C-NMR (75.5 MHz, CDCl₃): δ (ppm) 169.8 (C_{q}, C2), 161.9 (C_{q}, C6), 152.0 (C_{q}, C14), 147.7 (C_{q}, C4), 128.36 (2C, CH, C12/16) 125.3 (CH, C5), 121.2 (C_{q}, C11), 111.8 (2C, CH, C13/15), 61.4 (CH₂, C9), 40.4 (2C, CH₃, C18/19), 14.5 (CH₃, C10).

### Example 10: Ethyl 2-(4-isobutoxyphenyl)thiazole-4-carboxylate (AM-1-75)

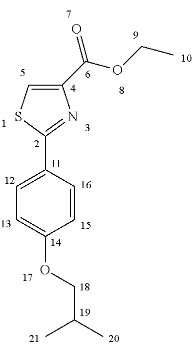 [SMILES: CCOC(=O)c1csc(-c2ccc(OCC(C)C)cc2)n1]

According to General Procedure A, ethyl 2-bromothiazole-4-carboxylate (1.0 eq, 805 μmol, 200 mg) was coupled with 4-isobutoxyphenylboronic acid (0.9 eq, 724 μmol, 140 mg). The crude product was purified via column chromatography (50 mL SiO₂, eluent CH/EA 9:1 to 4:1).

Yield: 172 mg (563 μmol, 78%) light yellow solid

C₁₆H₁₉NO₃S [305.39]

m.p.: 91° C.

R_{f}: 0.47 (CH/EA 4:1)

HR-MS [EI, M⁺]: calcd. 305.1086, found 305.1084.

¹H-NMR (300 MHz, CDCl₃): δ (ppm) 8.08 (s, 1H, H5), 7.93 (d, J=8.8 Hz, 2H, H12/16), 6.95 (d, J=8.8 Hz, 2H, C13/15), 4.44 (q, J=7.1 Hz, 2H, H9), 3.77 (d, J=6.5 Hz, 2H, H18), 2.11 (hept, J=6.6 Hz, 1H, H19), 1.43 (t, J=7.1 Hz, 3H, H10), 1.03 (d, J=8.9 Hz, 6H, H20/21).

¹³C-NMR (75.5 MHz, CDCl₃): δ (ppm) 169.0 (C_{q}, C2), 161.7 (C_{q}, C6), 161.5 (C_{q}, C14), 148.0 (C_{q}, C4), 128.64 (2C, CH, C12/16), 126.3 (CH, C5), 125.6 (C_{q}, 11), 114.9 (2C, CH, C13/15), 74.7 (CH₂, C18), 61.6 (CH₂, C9), 28.4 (CH, C19), 19.4 (2C, CH₃, C20/21), 14.5 (CH₃, C10).

### Example 105: Ethyl 2-(4-propoxyphenyl)thiazole-4-carboxylate (AM-1-82)

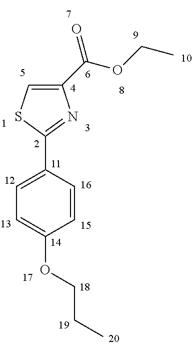 [SMILES: CCCOc1ccc(-c2nc(C(=O)OCC)cs2)cc1]

According to General Procedure A, ethyl 2-bromothiazole-4-carboxylate (1.0 eq, 678 μmol, 160 mg) was coupled with 4-propoxyyphenylboronic acid (1.05 eq, 724 μmol, 137 mg). The crude product was purified via preparative HPLC (method A).

Yield: 109 mg (374 μmol, 55%) colorless crystals

C₁₅H₁₇NO₃S [291.37]

m.p.: 97° C.

R_{f}: 0.36 (CH/EA 4:1)

HR-MS [EI, M⁺]: calcd. 291.0929, found 291.0933.

¹H-NMR (300 MHz, CDCl₃): δ (ppm) 8.08 (s, 1H, H5), 7.93 (d, J=8.8 Hz, 2H, H12/16), 6.94 (d, J=8.8 Hz, 2H, H13/15), 4.44 (q, J=7.1 Hz, 2H, H9), 3.97 (t, J=6.5 Hz, 2H, H18), 1.90-1.76 (m, 2H, H19), 1.42 (t, J=7.1 Hz, 3H, H10), 1.05 (t, J=7.4 Hz, 3H, H20).

¹³C-NMR (75.5 MHz, CDCl₃): δ (ppm) 169.0 (C_{q}, C2), 161.7 (C_{q}, C6), 161.4 (C_{q}, C14), 148.0 (C_{q}, C4), 128.7 (2C, CH, C12/16), 126.3 (CH, C5), 125.7 (C_{q}, C11), 114.9 (2C, CH, C13/15), 69.8 (CH₂, C18), 61.6 (CH₂, C9), 22.7 (CH₂, C19), 14.5 (CH₃, C10), 10.6 (CH₃, C20).

### Example 106: Ethyl 2-(4-isopropoxyphenyl)thiazole-4-carboxylate (AM-1-93a)

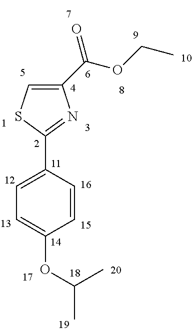 [SMILES: CCOC(=O)c1csc(-c2ccc(OC(C)C)cc2)n1]

According to General Procedure A, ethyl 2-bromothiazole-4-carboxylate (1.0 eq, 847 μmol, 200 mg) was coupled with 4-isopropoxyphenylboronic acid (0.9 eq, 770 μmol, 139 mg). The crude product was purified column chromatography (65 mL SiO₂, eluent CH/EA/DCM 10:1:1)

Yield: 164 mg (563 μmol, 73%) light yellow solid

C₁₅H₁₇NO₃S [291.37]

m.p.: 58° C.

R_{f}: 0.36 (CH/EA 4:1)

HR-MS [EI, M⁺]: calcd. 291.0929, found 291.0928.

¹H-NMR (300 MHz, CDCl₃): δ (ppm) 8.08 (s, 1H, H5), 7.93 (d, J=8.7 Hz, 2H, H12/16), 6.93 (d, J=8.6 Hz, 2H, H13/15), 4.62 (m, 1H, H18), 4.44 (q, J=7.1 Hz, 2H, C9), 1.42 (t, J=7.1 Hz, 3H, C10), 1.36 (d, J=6.0 Hz, 6H, C19/20).

¹³C-NMR (75.5 MHz, CDCl₃): δ (ppm) 169.0 (C_{q}, C2), 161.7 (C_{q}, C6), 160.2 (C_{q}, C14), 148.0 (C_{q}, C4), 128.7 (2C, C12/16), 126.3 (CH, C5), 125.5 (C_{q}, C11), 116.0 (2C, CH, C13/15), 70.2 (CH, C18), 61.6 (CH₂, C9), 22.1 (2C, CH₃, C19/20), 14.5 (CH₃, C10).

### Example 107: Ethyl 2-(4-(trifluoromethoxy)phenyl)thiazole-4-carboxylate (AM-1-93b)

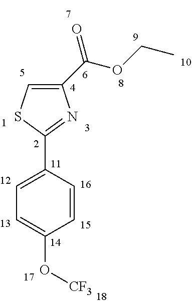 [SMILES: CCOC(=O)c1csc(-c2ccc(OC(F)(F)F)cc2)n1]

According to General Procedure A, ethyl 2-bromothiazole-4-carboxylate (1.0 eq, 847 μmol, 200 mg) was coupled with 4-trifluoromethoxyphenylboronic acid (0.9 eq, 770 μmol, 160 mg). The crude product was purified column chromatography (65 mL SiO₂, eluent CH/EA/DCM 10:1:1) and preparative HPLC (method A)

Yield: 56 mg (177 μmol, 23%) colorless solid

C₁₃H₁₀F₃NO₃S [317.28]

m.p.: 92° C.

R_{f}: 0.42 (CH/EA 4:1)

HR-MS [EI, M⁺]: calcd. 291.0929, found 291.0928.

¹H-NMR (300 MHz, CDCl₃): δ (ppm) 8.17 (s, 1H, H5), 8.05 (d, J=8.7 Hz, 2H, H12/16), 7.30 (d, J=8.2 Hz, 2H, H13/15), 4.45 (q, J=7.1 Hz, 2H; H9), 1.43 (t, J=7.1 Hz, 3H, H10).

¹³C-NMR (75.5 MHz, CDCl₃): δ (ppm) 167.3 (C_{q}, C2), 161.5 (C_{q}, C6), 151.0 (C_{q}, C14), 148.5 (C_{q}, C4), 131.5 (C_{q}, C11), 128.7 (2C, CH, C12/16), 127.5 (CH, C5), 122.2, 118.8 (d, J=258, CF₃), 121.4 (2C, CH, C13/15), 61.7 (CH₂, C9), 14.5 (CH₃, C10).

### Example 108: Ethyl 2-(4-(2,2,2-trifluoroethoxy)phenyl)thiazole-4-carboxylate (AM-1-98)

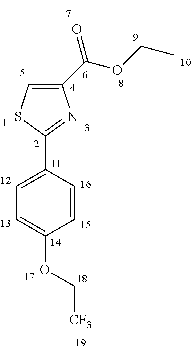 [SMILES: CCOC(=O)c1csc(-c2ccc(OCC(F)(F)F)cc2)n1]

According to General Procedure A, ethyl 2-bromothiazole-4-carboxylate (1.0 eq, 225 μmol, 53 mg) was coupled with (4-(2,2,2-trifluoroethoxy)phenyl)boronic acid (1.0 eq, 225 μmol, 50 mg). The crude product was purified column chromatography (25 mL SiO₂, eluent CH/EA/DCM 12:1:1 to 8:1:1).

Yield: 50 mg (151 μmol, 67%) off-white solid

C₁₄H₁₂F₃NO₃S [331.31]

m.p.: 119° C.

R_{f}: 0.15 (CH/EA 4:1)

HR-MS [EI, M⁺]: calcd. 331.0490, found 331.0491.

¹H-NMR (300 MHz, CDCl₃): δ (ppm) 8.11 (s, 1H, H5), 7.98 (d, J=8.8 Hz, 2H, H12/16), 7.01 (d, J=8.7 Hz, 2H, H13/15), 4.49-4.35 (m, 4H, H9/18), 1.43 (t, J=7.1 Hz, 3H, H10).

¹³C-NMR (75.5 MHz, CDCl₃): δ (ppm) 168.2 (C_{q}, C2), 161.6 (C_{q}, C6), 159.2 (C_{q}, C14), 148.2 (C_{q}, C4), 128.9 (2C, CH, C12/16), 127.7 (C_{q}, C11), 126.8 (CH, C5), 115.3 (2C, CH, C13/15), 66.10, 65.63 (d, J=36, CH₂, C18), 61.65 (CH₂, C9), 14.50 (CH₃, C10).

### Example 109: Ethyl 2-(4-butoxyphenyl)thiazole-4-carboxylate (AM-2-102a)

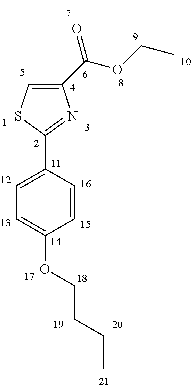 [SMILES: CCCCOc1ccc(-c2nc(C(=O)OCC)cs2)cc1]

According to General Procedure A, ethyl 2-bromothiazole-4-carboxylate (1.0 eq, 847 μmol, 200 mg) was coupled with 4-butoxyphenylboronic acid (1.0 eq, 847 μmol, 164 mg). The crude product was purified column chromatography (60 mL SiO₂, eluent CH/EA/DCM 18:1:1 to 12:1:1).

Yield: 95 mg (311 μmol, 77%) yellow solid

C₁₆H₁₉NO₃S [305.39]

m.p.: 64° C.

R_{f}: 0.37 (CH/EA 4:1)

HR-MS [EI, M⁺]: calcd. 305.1086, found 305.1087.

¹H-NMR (300 MHz, CDCl₃): δ (ppm) 8.08 (s, 1H, H5), 7.93 (d, J=8.7 Hz, 2H, H12/16), 6.94 (d, J=8.8 Hz, 2H, H13/15), 4.44 (q, J=7.1 Hz, 2H, H9), 4.01 (t, J=6.5 Hz, 2H, H18), 1.85-1.71 (m, 2H, H19), 1.52 (m, 2H, H20), 1.42 (t, J=7.1 Hz, 3H, H10), 0.98 (t, J=7.3 Hz, 3H, H21).

¹³C-NMR (75.5 MHz, CDCl₃): δ (ppm) 169.0 (C_{q}, C2), 161.7 (C_{q}, C6), 161.4 (C_{q}, C14), 148.0 (C_{q}, C4), 128.7 (2C, CH, C12/16), 126.3 (CH, C5), 125.7 (C_{q}, C11), 114.9 (2C, CH, C13/15), 68.0 (CH₂, C18), 61.6 (CH₂, C9), 31.4 (CH₂, C19), 19.4 (CH₂, C20), 14.5 (CH₃, C10), 14.0 (CH₃, C21).

### Example 110: Ethyl 2-(4-(pentyloxy)phenyl)thiazole-4-carboxylate (AM-2-102b)

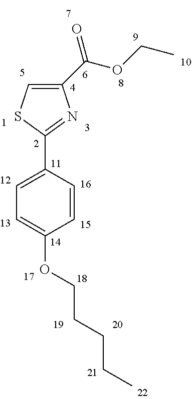 [SMILES: CCCCCOc1ccc(-c2nc(C(=O)OCC)cs2)cc1]

According to General Procedure A, ethyl 2-bromothiazole-4-carboxylate (1.0 eq, 847 μmol, 200 mg) was coupled with 4-pentyloxyphenylboronic acid (1.0 eq, 847 μmol, 176 mg). The crude product was purified column chromatography (60 mL SiO₂, eluent CH/EA/DCM 16:1:1 to 10:1:1).

Yield: 183 mg (311 μmol, 69%) yellow solid

C₁₇H₂₁NO₃S [319.42]

m.p.: 70° C.

R_{f}: 0.42 (CH/EA 4:1)

HR-MS [EI, M⁺]: calcd. 319.1242, found 319.1243.

¹H-NMR (300 MHz, CDCl₃): δ (ppm) 8.07 (s, 1H, H5), 7.93 (d, J=8.8 Hz, 2H, H12/16), 6.94 (d, J=8.8 Hz, 2H, H13/15), 4.44 (q, J=7.1 Hz, 2H, H9), 4.00 (t, J=6.5 Hz, 2H, H18), 1.81 (m, 2H, H19), 1.42 (m, 7H, H10/20/21), 0.94 (t, J=6.9 Hz, 3H, H22).

¹³C-NMR (75.5 MHz, CDCl₃): δ (ppm) 169.0 (C_{q}, C5), 161.7 (C_{q}, C6), 161.4 (C_{q}, C14), 148.0 (C_{q}, C4), 128.7 (2C, C12/16) 126.3 (CH, C5), 125.7 (C_{q}, C11), 114.9 (2C, CH, C13/15), 68.4 (CH₂, C18), 61.6 (CH₂, C9), 29.0 (CH₂, C19), 28.3 (CH₂, C20), 22.6 (CH₂, C21), 14.5 (CH₃, C10), 14.13 (CH₃, C22).

### Example 111: Ethyl 2-(4-(hexyloxy)phenyl)thiazole-4-carboxylate (AM-2-102c)

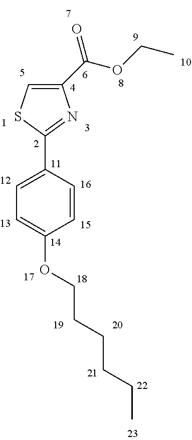 [SMILES: CCCCCCOc1ccc(-c2nc(C(=O)OCC)cs2)cc1]

According to General Procedure A, ethyl 2-bromothiazole-4-carboxylate (1.0 eq, 847 μmol, 200 mg) was coupled with 4-hexyloxyphenylboronic acid (1.0 eq, 847 μmol, 188 mg). The crude product was purified column chromatography (60 mL SiO₂, eluent CH/EA/DCM 18:1:1 to 12:1:1).

Yield: 192 mg (311 μmol, 68%) yellow solid

C₁₈H₂₃NO₃S [333.45]

m.p.: 70° C.

R_{f}: 0.40 (CH/EA 4:1)

HR-MS [EI, M⁺]: calcd. 333.1399, found 333.1396.

¹H-NMR (300 MHz, CDCl₃): δ (ppm) 8.07 (s, 1H, H5), 7.93 (d, J=8.7 Hz, 2H, H12/16), 6.94 (d, J=8.7 Hz, 2H, H13/15), 4.44 (q, J=7.1 Hz, 2H, H9), 4.00 (t, J=6.5 Hz, 2H, H18), 1.85-1.74 (m, 2H, H19), 1.55-1.45 (m, 2H, H20), 1.42 (t, J=7.1 Hz, 3H, H10), 1.39-1.30 (m, 4H, H21/22), 0.92 (t, J=6.6 Hz, 3H, H23).

¹³C-NMR (75.5 MHz, CDCl₃): δ (ppm) 169.0 (C_{q}, C2), 161.7 (C_{q}, C6), 161.4 (C_{q}, C14), 148.0 (C_{q}, C4), 128.7, (2C, CH, C12/16), 126.3 (CH, C5), 125.7 (C_{q}, C11), 114.9 (2C, CH, C13/15), 68.4 (CH₂, C18), 61.6 (CH₂, C9), 31.7 (CH₂, C21), 29.3 (CH₂, C19), 25.8 (CH₂, C20), 22.7 (CH₂, C22), 14.5 (CH₃, C10), 14.2 (CH₃, C23).

### Example 112: Ethyl 2-(4-(sec-butoxy)phenyl)thiazole-4-carboxylate (AM-1-103)

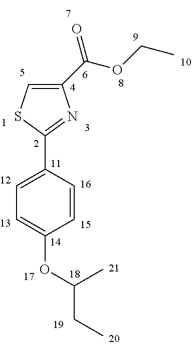 [SMILES: CCOC(=O)c1csc(-c2ccc(OC(C)CC)cc2)n1]

According to General Procedure A, ethyl 2-bromothiazole-4-carboxylate (1.0 eq, 773 μmol, 182 mg) was coupled with 4-(sec-butoxy)phenylboronic acid (1.0 eq., 773 μmol, 150 mg). The crude product was purified column chromatography (60 mL SiO₂, eluent CH/EA/DCM 12:1:1)

Yield: 90 mg (295 μmol, 38%) yellowish oil

C₁₆H₁₉NO₃S [305.39]

R_{f}: 0.42 (CH/EA 4:1)

HR-MS [EI, M⁺]: calcd. 305.1086, found 305.1089.

¹H-NMR (300 MHz, DMSO-d₆): δ (ppm) 8.46 (s, 1H, H5), 7.88 (d, J=8.5 Hz, 2H, H12/16), 7.04 (d, J=8.5 Hz, 2H, H13/15), 4.48 (td, J=11.4, 5.5 Hz, 1H, H18), 4.32 (q, J=7.0 Hz, 2H, H9), 1.72-1.56 (m, 2H, H19), 1.32 (t, J=7.1 Hz, 3H, H10), 1.25 (d, J=5.9 Hz, 3H, H21), 0.92 (t, J=7.3 Hz, 2H, H20).

¹³C-NMR (75.5 MHz, DMSO-d₆): δ (ppm) 167.7 (C_{q}, C2), 160.8 (C_{q}, C6), 159.9 (C_{q}, C14), 146.7 (C_{q}, C4), 128.2 (CH, C5), 128.1 (2C, CH, C12/16), 124.8 (C_{q}, C11), 116.0 (2C, CH, C13/15) 74.4 (CH, C18), 60.8 (CH₂, C9), 28.5 (CH₂, C19), 18.9 (CH₃, C21), 14.2 (CH₃, C10), 9.4 (CH₃, C20).

### Example 113: Ethyl 2-(4-(allyloxy)phenyl)thiazole-4-carboxylate (AM-1-109)

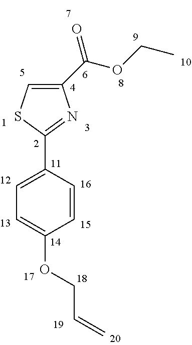 [SMILES: C=CCOc1ccc(-c2nc(C(=O)OCC)cs2)cc1]

According to General Procedure A, ethyl 2-bromothiazole-4-carboxylate (1.0 eq, 899 μmol, 212) was coupled with (4-(allyloxy)phenyl)boronic acid (1.0 eq., 899 μmol, 160 mg). The crude product was purified column chromatography (75 mL SiO₂, eluent CH/EA/DCM 12:1:1)

Yield: 90 mg (411 μmol, 46%) yellowish solid

C₁₅H₁₅NO₃S [289.3490]

### R_{f}: 0.38 (CH/EA 4:1)

m.p.: 58° C.

HR-MS [EI, M⁺]: calcd. 289.0773, found 289.0776.

¹H-NMR (300 MHz, DMSO-d₆): δ (ppm) 8.08 (s, 1H, H5), 7.94 (d, J=8.8 Hz, 2H, H12/16), 6.97 (d, J=8.8 Hz, 2H, H13/15), 6.06 (ddd, J=22.4, 10.5, 5.2 Hz, 1H, H19), 5.50-5.23 (m, 2H, H20), 4.59 (d, J=5.2 Hz, 2H, H18), 4.44 (q, J=7.1 Hz, 2H, H9), 1.42 (t, J=7.1 Hz, 3H, H10).

¹³C-NMR (75.5 MHz, DMSO-d): δ (ppm) 168.9 (C_{q}, C2), 161.7 (C_{q}, C6), 160.8 (C_{q}, C14), 148.0 (C_{q}, C4), 132.9 (CH, C19), 128.7 (2C, CH, C12/16), 126.4 (CH, C5), 126.0 (C_{q}, C11), 118.2 (CH₂, C20), 115.2 (2C, CH, C13/15), 69.0 (CH₂, C18), 61.6 (CH₂, C9), 14.5 (CH₃, C10).

### Intermediate: sec-Butyl 2-bromothiazole-4-carboxylate (AM-2-175a)

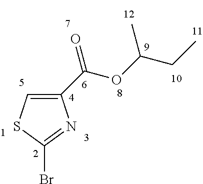

According to General Procedure B, 2-Bromothiazole-4-carboxylic acid (1.0 eq, 481 μmol, 100 mg) was esterified with 2-butanol.

Yield: 122 mg (462 μmol, 96%) off-white solid

C₈H₁₀BrNO₂S [264.14]

m.p.: 64° C.

R_{f}: 0.44 (CH/EA 4:1)

¹H-NMR (300 MHz, CDCl₃): δ (ppm) 8.08 (s, J=12.1 Hz, 1H, H5), 5.18-5.05 (m, 1H, H9), 1.83-1.60 (m, 2H, H10), 1.34 (d, J=6.3 Hz, 3H, H12), 0.95 (t, J=7.4 Hz, 3H, H11).

¹³C-NMR (75.5 MHz, CDCl₃): δ (ppm) 160.0 (C_{q}, C6), 147.9 (C_{q}, C2), 136.8 (C_{q}, C4), 130.6 (CH, C5), 74.3 (CH, C9), 29.9 (CH₂, C10), 19.6 (CH₃, C12), 9.9 (CH₃, C11).

### Intermediate: iso-Butyl 2-bromothiazole-4-carboxylate (AM-2-176)

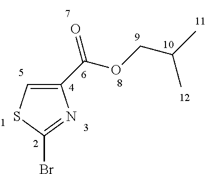

According to General Procedure B, 2-bromothiazole-4-carboxylic acid (1.0 eq, 913 μmol, 200 mg) was esterified with isobutanol.

Yield: 230 mg (871 μmol, 95%) beige solid

C₈H₁₀BrNO₂S [264.14]

R_{f}: 0.56 (CH/EA 2:1)

¹H-NMR (300 MHz, CDCl₃): δ (ppm) 8.09 (s, J=12.4 Hz, 1H, H5), 4.12 (d, J=6.8 Hz, 2H, H9), 2.20-2.00 (m, 1H, H10), 0.98 (d, J=6.7 Hz, 6H, H11/12).

¹³C-NMR (75.5 MHz, CDCl₃): δ (ppm) 160.2 (C_{q}, C6), 147.4 (C_{q}, C2), 136.9 (C_{q}, C4), 130.7 (CH, C5), 71.8 (CH₂, C9), 27.9 (CH, C10), 19.2 (2C, CH₃, C11/12).

### Example 114: sec-Butyl 2-(4-ethoxyphenyl)thiazole-4-carboxylate (AM-2-178a)

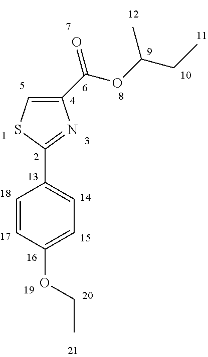 [SMILES: CCOc1ccc(-c2nc(C(=O)OC(C)CC)cs2)cc1]

According to general procedure A, AM-2-175a (1.0 eq., 340 μmol, 90 mg) was coupled with 4-ethoxyphenylboronic acid (1.74 eq, 590 μmol, 98 mg). The crude product was purified via column chromatography (40 mL SiO₂, eluent CH/EA 10:1).

Yield: 68 mg (223 μmol, 66%) orange solid

C₁₆H₁₉NO₃S [305.39]

m.p.: 54° C.

R_{f}: 0.49 (CH/EA 4:1)

¹H-NMR (300 MHz, CDCl₃): δ (ppm) 8.03 (s, 1H, H5), 7.94 (d, J=8.7 Hz, 2H, H14/18), 6.94 (d, J=8.7 Hz, 2H, H15/17), 5.13 (dd, J=12.5, 6.2 Hz, 1H, H9), 4.08 (q, J=6.9 Hz, 2H, H20), 1.73 (dtd, J=21.0, 13.9, 6.9 Hz, 2H, H10), 1.44 (t, J=6.9 Hz, 3H, H21), 1.36 (d, J=6.2 Hz, 3H, H12), 0.98 (t, J=7.4 Hz, 3H, H11).

¹³C-NMR (75.5 MHz, CDCl₃): δ (ppm) 168.8 (C_{q}, C2), 161.3 (C_{q}, C6), 161.2 (C_{q}, C16), 148.4 (C_{q}, C4), 128.7 (2C, CH, C14/18), 125.9 (CH, C5), 125.8 (C_{q}, C13) 114.9 (2C, CH, C15/17), 73.6 (CH, C9), 63.8 (CH₂, C20), 29.0 (CH₂, C10), 19.7 (CH₃, C12), 14.9 (CH₃, C21), 9.9 (CH₃, C11).

### Example 115: sec-Butyl 2-(4-ethoxyphenyl)thiazole-4-carboxylate (AM-2-178b)

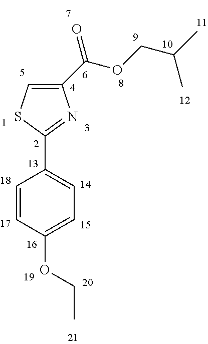 [SMILES: CCOc1ccc(-c2nc(C(=O)OCC(C)C)cs2)cc1]

According to general procedure A, AM-2-176 (1.0 eq., 379 μmol, 100 mg) was coupled with 4-ethoxyphenylboronic acid (1.5 eq, 568 μmol, 95 mg). The crude product was purified via column chromatography (50 mL SiO₂, eluent CH/EA 10:1).

Yield: 77 mg (252 μmol, 66%) orange solid

C₁₆H₁₉NO₃S [305.39]

m.p.: 100° C.

R_{f}: 0.49 (CH/EA 4:1)

¹H-NMR (300 MHz, CDCl₃): δ (ppm) 8.05 (s, 1H, H5), 7.94 (d, J=8.7 Hz, 2H, H14/18), 6.94 (d, J=8.7 Hz, 2H, H15/17), 4.15 (d, J=6.8 Hz, 2H, H9), 4.09 (q, J=7.0 Hz, 2H, H20), 2.11 (tt, J=15.6, 7.9 Hz, 1H, H10), 1.44 (t, J=7.0 Hz, 3H, H21), 1.02 (d, J=6.7 Hz, 6H, H11/12).

¹³C-NMR (75.5 MHz, CDCl₃): δ (ppm) 168.9 (C_{q}, C2), 161.6 (C_{q}, C6), 161.2 (C_{q}, C16), 148.0 (C_{q}, C4), 128.7 (2C, CH, C14/18), 126.1 (CH, C5), 125.8 (C_{q}, C13), 114.9 (2C, CH, C15/17), 71.4 (CH₂, C9), 63.8 (CH₂, C20), 28.0 (CH, C10), 19.3 (2C, CH₃, C11/12), 14.9 (CH₃, C21).

### Reference Compound: 2-(4-Ethoxyphenyl)thiazole-4-carboxylic acid (AM-2-177)

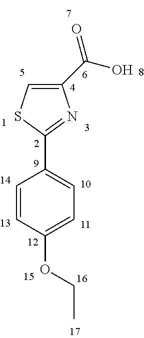 [SMILES: CCOc1ccc(-c2nc(C(=O)O)cs2)cc1]

In a 250 mL round bottom flask ethyl-(4-ethoxyphenyl)thiazole-2-carboxylate (1.0 eq., 2.16 mmol, 600 mg) was dissolved in 50 mL THF/MeOH/H₂O (5:4:1) and LiOH (5.0 eq, 10.8 mmol, 253 mg) was added. The reaction mixture was stirred at RT for 16 h. When full conversion was observed via TLC, the solvent was removed under reduced pressure. The residue was taken up in 50 mL H₂O, acidified to pH 2 and extracted with DCM (2×100 mL). The combined organic phases was washed with Brine, dried over Na₂SO₄, filtered and evaporated to dryness. The crude product was used in the next step without further purification.

Yield: 531 mg (2.13 μmol, 99%) off-white solid

C₁₂H₁₁NO₃S [249.28]

m.p.: 158° C.

R_{f}: 0.06 (CH/EA 2:1)

¹H-NMR (300 MHz, CDCl₃): δ (ppm) 9.81 (bs, 1H, H8), 8.19 (d, J=14.7 Hz, 1H, H5), 7.92 (d, J=8.7 Hz, 2H, H10/14), 6.96 (d, J=8.7 Hz, 2H, H11/13), 4.10 (q, J=6.9 Hz, 2H, H16), 1.45 (t, J=6.9 Hz, 3H, H17).

¹³C-NMR (75.5 MHz, CDCl₃): δ (ppm) 169.3 (C_{q}, C2), 163.8 (C_{q}, C6), 161.5 (C_{q}, C12), 146.7 (C_{q}, C4), 128.6 (2C, CH, C10/14), 127.4 (CH, C5), 125.2 (C_{q}, C9), 115.1 (2C, CH, C11/13), 63.9 (CH₂, C16), 14.9 (CH₃, C17).

### Example 116: 2,2,2-Trifluoroethyl 2-(4-ethoxyphenyl)thiazole-4-carboxylate (AM-2-180b)

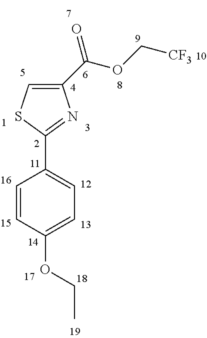 [SMILES: CCOc1ccc(-c2nc(C(=O)OCC(F)(F)F)cs2)cc1]

According to general procedure C, 2-(4-ethoxyphenyl)thiazole-4-carboxylic acid (1.0 eq., 301 μmol, 75 mg) was esterified with 2,2,2,-trifluoroethanol (1.5 eq, 451 μmol, 32 μL). The crude product was purified via column chromatography (40 mL SiO₂, eluent CH/EA 10:1 to 8:1) and preparative HPLC (method B).

Yield: 37 mg (112 μmol, 37%) colorless crystals

C₁₄H₁₂F₃NO₃S [331.31]

m.p.: 136° C.

R_{f}: 0.83 (CH/EA 1:1)

¹H-NMR (300 MHz, CDCl₃): δ (ppm) 8.18 (s, 1H, H5), 7.94 (d, J=8.7 Hz, 2H, H12/16), 6.95 (d, J=8.7 Hz, 2H, H13/15), 4.75 (q, J=8.3 Hz, 2H, H9), 4.09 (q, J=6.9 Hz, 2H, H18), 1.45 (t, J=6.9 Hz, 3H, H19).

¹³C-NMR (75.5 MHz, CDCl₃): δ (ppm) 169.5 (C_{q}, C2), 161.4 (C_{q}, C6), 159.7 (C_{q}, C14), 145.7 (C_{q}, C4), 128.7 (2C, CH, C12/16), 128.2 (CH, C5), 125.4 (C_{q}, C11), 125.0, 121.3 (d, J=123 Hz, CF₃), 115.0 (2C CH, C13/15), 63.87 (CH₂, C18), 61.2, 60.7 (d, J=61 Hz, CH₂, C9), 14.9 (CH₃, C19).

### Example 117: Pentan-2-yl 2-(4-ethoxyphenyl)thiazole-4-carboxylate (AM-3-183b)

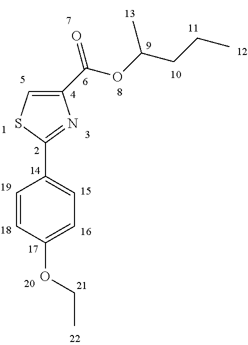 [SMILES: CCCC(C)OC(=O)c1csc(-c2ccc(OCC)cc2)n1]

According to general procedure C, 2-(4-ethoxyphenyl)thiazole-4-carboxylic acid (1.0 eq., 301 μmol, 75 mg) was esterified with 2-pentanol (1.5 eq, 451 μmol, 49 μL). The crude product was purified via column chromatography (45 mL SiO₂, eluent CH/EA/DCM 14:1:1).

Yield: 81 mg (254 μmol, 84%) colorless liquid

C₁₇H₂₁NO₃S [319.42]

m.p.: 136° C.

R_{f}: 0.86 (CH/EA 1:1)

¹H-NMR (300 MHz, CDCl₃): δ (ppm) 8.02 (s, 1H, H5), 7.94 (d, J=8.7 Hz, 2H, H15/19), 6.94 (d, J=8.7 Hz, 2H, H16/18), 5.21 (dd, J=12.6, 6.2 Hz, 1H, H9), 4.09 (q, J=6.9 Hz, 2H, H21), 1.76-1.54 (m, 2H, H10), 1.44 (t, J=6.9 Hz, 5H, H11/21), 1.36 (d, J=6.2 Hz, 3H, H13), 0.95 (t, J=7.2 Hz, 3H, H12).

¹³C-NMR (75.5 MHz, CDCl₃): δ (ppm) 168.8 (C_{q}, C2), 161.3 (C_{q}, C6), 161.2 (C_{q}, C17), 148.4 (C_{q}, C4), 128.7 (2C, CH, C15/19), 125.9 (CH, C5), 125.8 (C_{q}, C14), 114.9 (2C, CH, C16/18), 72.2 (CH, C9), 63.8 (CH₂, C21), 38.3 (CH₂, C10), 20.2 (CH₃, C13), 18.9 (CH₂, C11), 14.8 (CH₃, C22), 14.1 (CH₃, C12).

### Example 118: tert-Pentyl 2-(4-ethoxyphenyl)thiazole-4-carboxylate (AM-3-190a)

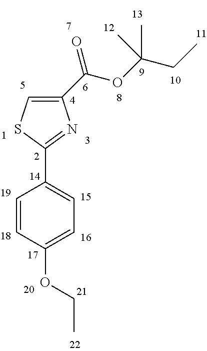 [SMILES: CCOc1ccc(-c2nc(C(=O)OC(C)(C)CC)cs2)cc1]

According to general procedure C, 2-(4-ethoxyphenyl)thiazole-4-carboxylic acid (1.0 eq., 301 μmol, 75 mg) was esterified with tert-pentanol (1.5 eq, 451 μmol, 49 μL). The crude product was purified via column chromatography (35 mL SiO₂, eluent CH/EA 10:1) and preparative HPLC (method B).

Yield: 67 mg (210 μmol, 70%) colorless solid

C₁₇H₂₁NO₃S [319.42]

m.p.: 75° C.

R_{f}: 0.84 (CH/EA 1:1)

¹H-NMR (300 MHz, CDCl₃): δ (ppm) 7.94 (m, 3H, H5/15/19), 6.93 (d, J=8.7 Hz, 2H, H16/18), 4.08 (q, J=6.9 Hz, 2H, H21), 1.94 (q, J=7.4 Hz, 2H, H10), 1.59 (s, 6H, H12/13), 1.43 (t, J=6.9 Hz, 3H, H22), 0.98 (t, J=7.4 Hz, 3H, H11).

¹³C-NMR (75.5 MHz, CDCl₃): δ (ppm) 168.5 (C_{q}, C2), 161.1 (C_{q}, 06), 160.6 (C_{q}, C17), 149.3 (C_{q}, C4), 128.6 (2C, CH, C15/19), 125.9 (C_{q}, C14), 125.3 (CH, C5), 114.8 (2C, CH, C16/18), 84.5 (C_{q}, C9), 63.8 (CH₂, C21), 33.9 (CH₂, C10), 25.8 (2C, CH₃, C12/13), 14.9 (CH₃, C22), 8.5 (CH₃, C11).

### Example 119: 3-Methoxypropyl 2-(4-ethoxyphenyl)thiazole-4-carboxylate (AM-3-213b)

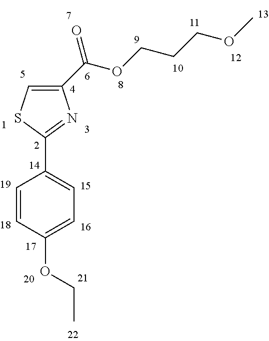 [SMILES: CCOc1ccc(-c2nc(C(=O)OCCCOC)cs2)cc1]

According to general procedure C, 2-(4-ethoxyphenyl)thiazole-4-carboxylic acid (1.0 eq., 301 μmol, 75 mg) was esterified with 3-methoxypropanol (1.5 eq, 451 μmol, 43 μL). The crude product was purified via column chromatography (35 mL SiO₂, eluent CH/EA 10:1) and preparative HPLC (method B).

Yield: 67 mg (210 μmol, 70%) colorless solid

C₁₅H₁₉NO₄S [321.39]

m.p.: 58° C.

R_{f}: 0.60 (CH/EA 1:1)

¹H-NMR (300 MHz, CDCl₃): δ (ppm) 8.07 (s, 1H, H5), 7.93 (d, J=8.8 Hz, 2H, H15/19), 6.94 (d, J=8.8 Hz, 2H, H16/18), 4.46 (t, J=6.5 Hz, 2H, H9), 4.09 (q, J=7.0 Hz, 2H, H21), 3.54 (t, J=6.2 Hz, 2H, H11), 3.36 (s, 3H, H13), 2.12-1.98 (m, 2H, H10), 1.44 (t, J=7.0 Hz, 3H, H22).

¹³C-NMR (75.5 MHz, CDCl₃): δ (ppm) 169.0 (C_{q}, C2), 161.6 (C_{q}, C6), 161.2 (C_{q}, C17), 147.8 (C_{q}, C4), 128.7 (2C, CH, C15/19), 126.4 (CH, C5), 125.7 (C_{q}, C14), 114.9 (2C, CH, C16/18), 69.3 (CH₂, C11), 63.8 (CH₂, C21), 62.7 (CH₂, C9), 58.9 (CH₃, C13), 29.2 (CH₂, C10), 14.9 (CH₃, C22).

### Example 120: 2-Methylallyl 2-(4-ethoxyphenyl)thiazole-4-carboxylate (AMU-18)

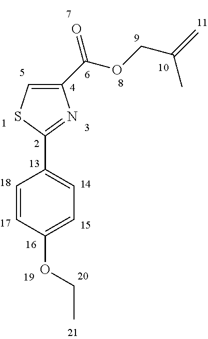 [SMILES: C=C(C)COC(=O)c1csc(-c2ccc(OCC)cc2)n1]

According to general procedure C, 2-(4-ethoxyphenyl)thiazole-4-carboxylic acid (1.0 eq., 401 μmol, 100 mg) was esterified with R-methylallyl alcohol (1.5 eq, 602 μmol, 50 μL). The crude product was purified via column chromatography (90 mL SiO₂, eluent CH/EA 15:1).

Yield: 55 mg (181 μmol, 45%) colorless solid

C₁₆H₁₇NO₃S [303.38]

m.p.: 88° C.

R_{f}: 0.20 (CH/EA 15:1)

¹H-NMR (300 MHz, CDCl₃): δ (ppm) 8.09 (s, 1H, H5), 7.94 (d, J=8.7 Hz, 2H, H14/18), 6.94 (d, J=8.7 Hz, 2H, H15/17), 5.04 (d, J=30.6 Hz, 2H, H, H11), 4.80 (s, 2H, H9), 4.09 (q, J=6.9 Hz, 2H, H20), 1.84 (s, 3H, H12), 1.44 (t, J=6.9 Hz, 3H, H21).

¹³C-NMR (75.5 MHz, CDCl₃): δ (ppm) 169.0 (C_{q}, C2), 161.2 (C_{q}, C16), 147.6 (C_{q}, C6), 139.86, 128.67, 126.5 (CH, C5), 125.7 (C_{q}, C13), 114.9 (2C, CH₂, C15/17), 113.4 (CH₂, C11), 68.4 (CH₂, C9), 63.8 (CH₂, C20), 19.7 (CH₃, C12), 14.9 (CH₃, C21).

### Example 121: (E)-but-2-en-1-yl 2-(4-ethoxyphenyl)thiazole-4-carboxylate (AMU-19)

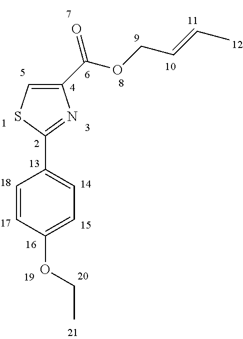 [SMILES: C/C=C/COC(=O)c1csc(-c2ccc(OCC)cc2)n1]

According to general procedure C, 2-(4-ethoxyphenyl)thiazole-4-carboxylic acid (1.0 eq., 401 μmol, 100 mg) was esterified with crotyl alcohol (1.5 eq, 602 μmol, 51 μL). The crude product was purified via column chromatography (90 mL SiO₂, eluent CH/EA 15:1).

Yield: 53 mg (170 μmol, 41%) colorless solid

C₁₆H₁₇NO₃S [303.38]

m.p.: 73° C.

R_{f}: 0.20 (CH/EA 15:1)

¹H-NMR (300 MHz, CDCl₃): δ (ppm) 8.08 (s, 1H, H5), 7.93 (d, J=8.8 Hz, 2H, H14/18), 6.94 (d, J=8.7 Hz, 2H, H15/17), 5.90 (dt, J=21.3, 6.4 Hz, 1H, H10), 5.82-5.63 (m, 1H, H11), 4.80 (d, J=6.4 Hz, 2H, H9), 4.08 (q, J=6.9 Hz, 2H, H20), 1.75 (d, J=6.1 Hz, 3H, H12), 1.44 (t, J=7.0 Hz, 3H, H21).

¹³C-NMR (75.5 MHz, CDCl₃): δ (ppm) 169.0 (C_{q}, C2), 161.5 (C_{q}, C6), 161.2 (C_{q}, C16), 147.9 (C_{q}, C4), 132.2 (CH, C10), 128.7 (2C, CH, C14/18), 126.5 (CH, C5), 125.7 (C_{q}, C13), 125.1 (CH, C11), 114.9 (2C, CH, C15/17), 66.2 (CH₂, C9), 63.8 (CH₂, C20), 18.0 (CH₃, C12), 14.9 (CH₃, C21).

### Example 122: Methyl 2-(4-ethoxyphenyl) thiazole-4-carboxylate (NP22c)

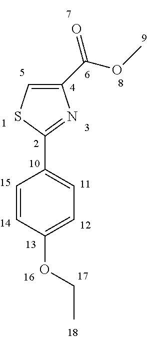 [SMILES: CCOc1ccc(-c2nc(C(=O)OC)cs2)cc1]

According to general procedure A, methyl 2-bromothiazole-4-carboxylate (1.0 eq., 0.45 mmol, 101 mg) was coupled with 4-ethoxyphenylboronic acid (0.9 eq, 0.47 μmol, 67 mg). The crude product was purified via column chromatography (32 mL SiO₂, eluent CH/EA/DMC 8:1:1).

Yield: 60 mg (228 μmol, 50%) beige solid

C₁₃H₁₃NO₃S [263.31]

m.p.: 70° C.

R_{f}: 0.33 (CH/EA 4:1)

¹H-NMR (300 MHz, CDCl₃): δ (ppm) 8.10 (s, 1H, C5), 7.93 (d, J=8.7 Hz, 2H, C11/15), 6.94 (d, J=8.7 Hz, 2H, C12/14), 4.09 (q, J=6.9 Hz, 2H, C17), 3.97 (s, 3H, C9), 1.44 (t, J=7.0 Hz, 3H, C18).

¹³C-NMR (75.5 MHz, CDCl₃): δ (ppm) 169.1 (C_{q}, C2), 162.2 (C_{q}, C6), 161.3 (C_{q}, C13), 147.6 (C_{q}, C4), 128.7 (C_{q}, C10), 126.6 (2C, CH, C11/15), 125.6 (CH, C5), 114.9 (2C, CH, C12/14), 63.8 (CH₂, C17), 52.6 (CH₃, C9), 14.9 (CH₃, C18).

### Example 123: Isopropyl 2-(4-ethoxyphenyl) thiazole-4-carboxylate (NP22d)

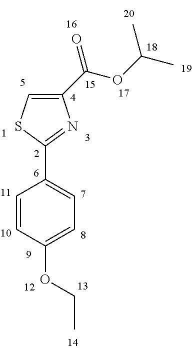 [SMILES: CCOc1ccc(-c2nc(C(=O)OC(C)C)cs2)cc1]

According to general procedure A, isopropyl 2-bromothiazole-4-carboxylate (1.0 eq., 0.40 mmol, 100 mg) was coupled with 4-ethoxyphenylboronic acid (0.9 eq, 0.36 mmol, 60 mg). The crude product was purified via column chromatography (26 mL SiO₂, eluent CH/EA/DMC 10:1:1).

Yield: 50 mg (172 μmol, 43%) yellowish solid

C₁₅H₁₇NO₃S [291.37]

m.p.: 95° C.

R_{f}: 0.38 (CH/EA 4:1)

¹H-NMR (300 MHz, CDCl₃): δ (ppm) 8.04 (s, 1H, C5), 7.94 (d, J=8.7 Hz, 2H, C7/11), 6.94 (d, J=8.7 Hz, 2H, C-8/10), 5.38-5.20 (m, 1H, C18), 4.09 (q, J=6.9 Hz, 2H, C-13), 1.42 (m, 9H, C14/19/20).

¹³C-NMR (75.5 MHz, CDCl₃): δ 168.8 (C_{q}, C2), 161.2 (C_{q}, C15), 161.2 (C_{q}, C9), 148.4 (C_{q}, C4), 128.7 (2C, CH, C7/11), 126.0 (CH, C5), 125.8 (C_{q}, C6), 114.9 (2C, CH, C8/10), 69.1 (CH, C18), 63.8 (CH₂, C13), 22.1 (2C, CH₃, C19/20), 14.9 (CH₃, C14) ppm.

### Intermediate: Ethyl 2-diazo-3-oxobutanoate (AM-59)

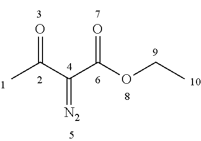

In an inert 100 mL round bottom flask equipped with Schlenk adapter and magnetic stirring bar 4-acetamidobenzenesulfonylazide (1.1 eq, 6.34 mmol, 1.57 g) was dissolved in 40 mL MeCN abs and cooled to 0° C. Ethyl acetoacetate (1.0 eq., 5.76 mmol, 740 μL) was added via syringe. Et₃N (3.0 eq, 17.3 mmol, 2.4 mL) was slowly added via syringe over 5 minutes. After further 10 minutes, the cooling bath was removed and the reaction mixture was stirred at RT overnight. The reaction mixture was filtered and the solvent removed under reduced pressure. The solid residue was taken up in Et₂O and triturated. It was filtered and the solvent was removed under reduced pressure. The crude product was used in the next step without further purification. It contained 20% sulfonamide.

Yield: 680 mg (impure, 3.49 mmol, 61%) yellow liquid

C₆H₈N₂O₃[156.05]

R_{f}: 0.39 (CH/EA 4:1)

¹H-NMR (300 MHz, CDCl₃): δ 4.30 (q, J=7.1 Hz, 2H, H9), 2.48 (s, 3H, H1), 1.33 (t, J=7.1 Hz, 3H, H10).

¹³C-NMR (75.5 MHz, CDCl₃): δ 190.4 (C_{q}, C2), 161.6 (C_{q}, C6), 61.6 (CH₂, C9), 28.4 (CH₃, C1), 14.5 (CH₃, C10).

### Intermediate: Ethyl 2-(4-ethoxy (AM-61)

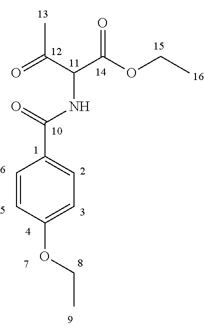 [SMILES: CCOC(=O)C(NC(=O)c1ccc(OCC)cc1)C(C)=O]

In an inert 25 ml two-neck round-bottom-flask equipped with air condenser with gas inlet and bubbler and magnetic stirring bar 4-ethoxybenzamide (1.0 eq, 2.42 mmol, 400 mg) and Rh₂(OAc)₄ (2.5 mol %, 58 μmol, 26 mg) were dissolved in 5 mL DCE and heated to reflux. A solution of AM-59 (1.44 eq, 3.48 mmol, 680 mg) in 2.5 mL DCE was added to the reaction mixture over a septum via syringe pump ((187.5 μL/h) under a flow of Ar. When the addition was completed, the reaction mixture was further stirred at reflux for 3 h. When full conversion was observed via TLC, the reaction mixture was cooled down to RT and the solvent was removed under reduced pressure. The crude product was purified via column chromatography (100 mL SiO2, eluent CH/EA 4:1 to 3:1 to 2:1).

Yield: 443 mg (1.51 mmol, 62%) yellow oil

C₁₅H₁₉NO₅ [293.13]

R_{f}: 0.57 (CH/EA 1:1)

¹H-NMR (300 MHz, CDCl₃): δ (ppm) 7.80 (d, J=8.7 Hz, 2H, H2/6), 7.20 (d, J=5.2 Hz, 1H, NH), 6.92 (d, J=8.7 Hz, 2H, H3/5), 5.41 (d, J=6.2 Hz, 1H, H11), 4.30 (q, J=7.1 Hz, 2H, H15), 4.08 (q, J=6.9 Hz, 2H, H8), 2.44 (s, 3H, H13), 1.43 (t, J=7.0 Hz, 3H, H9), 1.32 (t, J=7.1 Hz, 3H, H16).

¹³C-NMR (75.5 MHz, CDCl₃): δ (ppm) 199.0 (C_{q}, C12), 166.5 (C_{q}, C14), 166.4 (C_{q}, C10), 162.3 (C_{q}, C4), 129.3 (2C, CH, C2/6), 125.2 (C_{q}, C1), 114.5 (2C, CH, C3/5), 63.8 (CH₂, C8), 63.7 (CH, C11), 62.8 (CH₂, C15), 28.3 (CH₃, C13), 14.8 (CH₃, C9), 14.2 (CH₃, C16).

### Example 124: Ethyl 2-(4-ethoxyphenyl)-5-methylthiazole-4-carboxylate (AM-62)

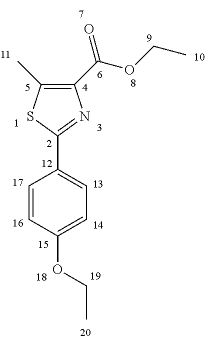 [SMILES: CCOC(=O)c1nc(-c2ccc(OCC)cc2)sc1C]

In an inert 100 mL Schlenk flask Lawesson's reagent (2.0 eq, 2.72 mmol, 1.1 g) and AM-61 (1.0 eq, 1.36 mmol, 400 mg) were dissolved in 10 mL THF abs and heated to reflux. When full conversion was observed via TLC after 3 h, the reaction mixture was cooled to RT and the solvent was removed under reduced pressure. The crude product was purified via column chromatography (100 mL SiO₂, eluent CH/EA 9:1).

Yield: 232 mg (797 μmol, 58%) yellowish solid

C₁₅H₁₇NO₃S [291.09]

m.p. 78° C.

R_{f}: 0.50 (CH/EA 4:1)

HR-MS [EI, M⁺]: calcd. 291.0929, found 291.0920.

¹H-NMR (300 MHz, CDCl₃): δ 7.85 (d, J=8.8 Hz, 2H, H13/17), 6.92 (d, J=8.8 Hz, 2H, H14/16), 4.43 (q, J=7.1 Hz, 2H, H9), 4.08 (q, J=6.9 Hz, 2H, H19), 2.78 (s, 3H, H11), 1.43 (t, J=7.0 Hz, 6H, H10/20).

¹³C-NMR (75.5 MHz, CDCl₃): δ (ppm) 163.9 (C_{q}, C2), 162.9 (C_{q}, C6), 160.9 (C_{q}, C15), 143.7 (C_{q}, C4), 142.1 (C_{q}, C5), 128.3 (C_{q}, C12), 125.9 (2C, CH, C13/17), 114.8 (2C, CH, C14/16), 63.8 (CH₂, C19), 61.2 (CH₂, C9), 14.9 (CH₃, C20), 14.5 (CH₃, C10), 13.4 (CH₃, C11).

### Intermediate: Ethyl 2-bromo-5-chlorothiazole-4-carboxylate (AM-2-135)

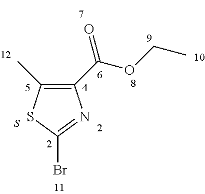

In an inert 100 mL Schlenk flask CuBr₂ (1.2 eq., 11.85 mmol, 2.65 g) was dried in vacuum for 1 h. AM-1-133 (1.0 eq, 9.87 mmol, 2.4 g) was added and dissolved in MeCN abs (70 mL). The reaction mixture was heated to 60° C. ^{t}BuONO (1.5 eq., 14.81 mmol, 2.0 mL) was added dropwise over septum via syringe, upon addition gas evolution was observed. Full conversion was observed via TLC and GC-MS after 30 min and the reaction mixture was cooled down to RT. It was diluted with 100 mL DCM and quenched via addition of 100 mL HCl 1 M. The phases were separated and the aqueous phase was extracted with DCM (2×100 mL). The combined organic phases were washed with Brine (1×100 mL), dried over Na₂SO₄, filtered and the solvent was removed under reduced pressure. The crude product was purified via filtration over a pad of silica (eluent DCM/EA 19:1).

Yield: 2.23 g (8.24 mmol, 84%) brownish solid

C₆H₅BrClNO₂S [270.53]

m.p.: 63° C.

R_{f}: 0.62 (CH/EA 4:1)

¹H-NMR (300 MHz, CDCl₃): δ (ppm) 4.43 (q, J=7.1 Hz, 2H, H9), 1.40 (t, J=7.1 Hz, 3H, H10).

¹³C-NMR (75.5 MHz, CDCl₃): δ (ppm) 159.5 (C_{q}, C6), 141.6 (C_{q}, C2), 132.7 (C_{q}, C5), 62.2 (CH₂, C9), 14.4 (CH₃, C10).

### Example 125: Ethyl 5-chloro-2-(4-ethoxyphenyl)thiazole-4-carboxylate (AM-2-137)

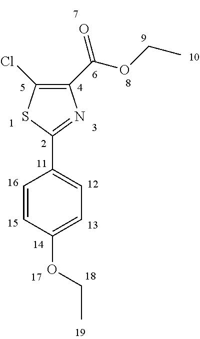 [SMILES: CCOC(=O)c1nc(-c2ccc(OCC)cc2)sc1Cl]

According to General Procedure A, AM-2-135 (1.0 eq, 1.85 mmol, 500 mg) was coupled with 4-ethoxyphenylboronic acid (1.0 eq., 1.85 mmol, 307 mg). The crude product was purified column chromatography (120 mL SiO₂, eluent CH/EA 12:1).

Yield: 287 mg (0.92 mmol, 50%) colorless solid

C₁₄H₁₄ClNO₃S [311.78]

m.p. 91° C.

R_{f}: 0.50 (CH/EA 4:1)

HR-MS [EI, M⁺]: calcd. 311.0383, found 311.0380.

¹H-NMR (300 MHz, CDCl₃): δ (ppm) 7.81 (d, J=8.7 Hz, 2H, H12/16), 6.93 (d, J=8.7 Hz, 2H, H13/15), 4.45 (q, J=7.1 Hz, 2H, H9), 4.08 (q, J=6.9 Hz, 2H, H18), 1.44 (t, J=7.0 Hz, 6H, H10/19).

¹³C-NMR (75.5 MHz, CDCl₃): δ (ppm) 164.7 (C_{q}, C2), 161.5 (C_{q}, C6), 161.1 (C_{q}, C14), 141.8 (C_{q}, C4), 133.0 (C_{q}, C5), 128.3 (2C, CH, C12/16), 125.2 (C_{q}, C11), 115.0 (2C, CH, C13/15), 63.9 (CH₂, C18), 61.8 (CH₂, C9), 14.8 (CH₃, C19), 14.4 (CH₃, C10).

### Example 126: Ethyl 2-(4-ethoxyphenyl)-5-methoxythiazole-4-carboxylate (AM-2-139)

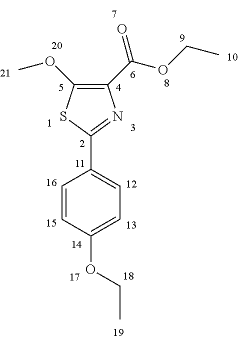 [SMILES: CCOC(=O)c1nc(-c2ccc(OCC)cc2)sc1OC]

In an inert Schienk flask Na (4.3 eq., 2.74 mmol, 63 mg) was dissolved in 7 mL MeOH abs. The solution was cooled down to 0° C. and AM-2-137 (1.0 eq, 641 μmol, 200 mg) was added. The reaction was stirred at RT, and after no conversion was observed after 1 h, it was further stirred at 55° C. After 21 h the reaction was quenched via the addition of water. The aqueous phase was neutralized and extracted with DCM (2×15 mL). The combined organic phase was washed with Brine (1×20 mL), dried over Na₂SO₄, filtered and the solvent was removed under reduced pressure. The flask containing the intermediate product was equipped with Schienk adapter and stirring bar and evacuated and backfilled with N₂ for three times. The intermediate was dissolved in 7 mL EtOH abs and Ti(OⁱPr)₄ was added via syringe (0.1 eq., 64 μmol, 20 μL). The reaction mixture was stirred at 90° C. overnight. When full conversion was observed via TLC, the reaction was cooled down to RT and quenched via the addition of NH₄Cl sat. It was extracted with EA (3×15 mL). The combined organic phases were washed with NaHCO₃ sat (1×15 mL) and Brine (1×15 mL), dried over Na₂SO₄, filtered and the solvent was removed under reduced pressure. The crude product was purified via preparative HPLC (method D).

Yield: 59 mg (192 μmol, 30%) colorless solid

C₁₅H₁₇NO₄S [307.09]

m.p. 120° C.

R_{f}: 0.36 (CH/EA 4:1)

¹H-NMR (300 MHz, CDCl₃): δ (ppm) 7.77 (d, J=8.8 Hz, 2H, H12/16), 6.91 (d, J=8.7 Hz, 2H, H13/15), 4.41 (q, J=7.1 Hz, 2H, H9), 4.13 (s, 3H, H21), 4.07 (q, J=7.0 Hz, 2H, H18), 1.42 (m, 6H, H10/19).

¹³C-NMR (75.5 MHz, CDCl₃): δ (ppm) 169.2 (C_{q}, C2), 161.9 (C_{q}, C6), 160.7 (C_{q}, C14), 152.8 (C_{q}, C4), 127.7 (2C, CH, C12/16), 126.7 (C_{q}, C5), 126.1 (C_{q}, C11), 114.8 (2C, CH, C13/15), 64.7 (CH₃, C21), 63.8 (CH₂, C18), 61.0 (CH₂, C9), 14.9 (CH₃, C19), 14.6 (CH₃, C10).

### Example 127: Cyclopentyl 2-(4-ethoxyphenyl)thiazole-4-carboxylate (AM-2-179)

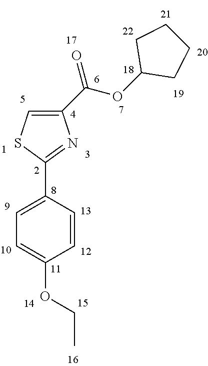 [SMILES: CCOc1ccc(-c2nc(C(=O)OC3CCCC3)cs2)cc1]

According to General Procedure C, 2-(4-ethoxyphenyl)thiazole-4-carboxylic acid (1.0 eq., 401 μmol, 100 mg) was esterified with cyclopentanol (1.5 eq, 602 μmol, 50 μL). The crude product was purified via column chromatography (50 mL SiO₂, eluent CH/EA/DCM 15:1:1).

Yield: 49 mg (154 μmol, 39%) colorless crystals

C₁₇H₁₉NO₃S [317.40]

R_{f}: 0.82 (CH/EA 1:1)

¹H-NMR (300 MHz, CDCl₃): δ 8.00 (s, 1H, H5), 7.94 (d, J=8.7 Hz, 2H, H9/13), 6.94 (d, J=8.7 Hz, 2H, H10/12), 5.52-5.33 (m, 1H, H18), 4.09 (q, J=6.9 Hz, 2H, H15), 2.08-1.65 (m, 8H, H19/20/21/22), 1.44 (t, J=6.9 Hz, 3H, H16).

¹³C-NMR (75.5 MHz, CDCl₃): δ 168.8 (C_{q}, C2), 161.4 (C_{q}, C6), 161.2 (C_{q}, C11), 148.4 (C_{q}, C4), 128.7 (2C, CH, C9/13), 125.9 (CH, C5), 125.8 (C_{q}, C8), 114.9 (2C, CH, C10/12), 78.3 (CH, C18), 63.8 (CH₂, C15), 32.9 (2C, CH₂, C19/22), 24.0 (2C, CH₂, C20/21), 14.9 (CH₃, C16).

### Example 128: 1,1,1,3,3,3-Hexafluoropropan-2-yl 2-(4-ethoxyphenyl)thiazole-4-carboxylate (AM-2-180a)

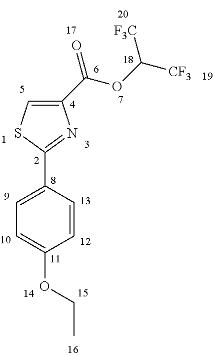 [SMILES: CCOc1ccc(-c2nc(C(=O)OC(C(F)(F)F)C(F)(F)F)cs2)cc1]

According to General Procedure C, 2-(4-ethoxyphenyl)thiazole-4-carboxylic acid (1.0 eq., 301 μmol, 75 mg) was esterified with 1,1,1,3,3,3-hexafluoropropan-2-ol (1.5 eq, 451 μmol, 47 μL). The crude product was purified via column chromatography (50 mL SiO₂, eluent CH/EA/DCM 18:1:1) and preparative HPLC (method E).

Yield: 43 mg (154 μmol, 36%) colorless powder

C₁₅H₁₁F₆NO₃S [399.31]

m.p.: 113° C.

R_{f}: 0.88 (CH/EA 1:1)

¹H-NMR (300 MHz, CDCl₃): δ 8.28 (s, 1H, H5), 7.95 (d, J=8.7 Hz, 2H, H9/13), 6.96 (d, J=8.6 Hz, 2H, H10/12), 6.04 (q, J=5.9 Hz, 1H, H18), 4.10 (q, J=6.9 Hz, 2H, H15), 1.45 (t, J=6.9 Hz, 3H, H16).

¹³C-NMR (75.5 MHz, CDCl₃): δ 170.0 (C_{q}, C2), 161.6 (C_{q}, C11), 157.7 (C_{q}, C6), 144.1 (C_{q}, C4), 129.8 (CH, C5), 128.8 (2C, CH, C9/13), 125.2 (C_{q}, C8), 115.1 (2C, CH, C10/12), 67.1 (CH, C18), 63.9 (CH₂, C15), 14.9 (CH₃, C16).

### Example 129: Cyclobutyl 2-(4-ethoxyphenyl)thiazole-4-carboxylate (AM-3-183a)

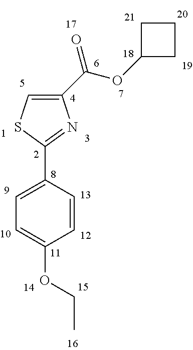 [SMILES: CCOc1ccc(-c2nc(C(=O)OC3CCC3)cs2)cc1]

According to general procedure C, 2-(4-ethoxyphenyl)thiazole-4-carboxylic acid (1.0 eq., 301 μmol, 75 mg) was esterified with cyclobutanol (1.5 eq, 451 μmol, 35 μL). The crude product was purified via column chromatography (40 mL SiO₂, eluent CH/EA/DCM 10:1:1) and preparative HPLC (method B).

Yield: 63 mg (208 μmol, 69%) off-white solid

C₁₆H₁₇NO₃S [303.38]

m.p.: 115° C.

R_{f}: 0.83 (CH/EA 1:1)

¹H-NMR (300 MHz, CDCl₃): δ 8.07 (s, 1H, H5), 7.93 (d, J=8.7 Hz, 2H, H9/13), 6.94 (d, J=8.7 Hz, 2H, H10/12), 5.33-5.15 (m, 1H, H18), 4.08 (q, J=6.9 Hz, 2H, H15), 2.56-2.37 (m, 2H, H19/21), 2.36-2.18 (m, 2H, H19/21), 1.86 (dd, J=20.1, 10.0 Hz, 1H, H20), 1.70 (m, 1H, H20), 1.44 (t, J=6.9 Hz, 3H, H16).

¹³C-NMR (75.5 MHz, CDCl₃): δ 168.9 (C_{q}, C2), 161.2 (C_{q}, C11), 161.0 (C_{q}, C6), 147.9 (C_{q}, C4), 128.7 (2C, CH, C9/13), 126.3 (CH, C5), 125.7 (C_{q}, C8), 114.9 (2C, CH, C10/12), 70.0 (CH, C18), 63.8 (CH₂, C15), 30.6 (2C, CH₂, C19/21), 14.9 (CH₂, C16), 13.7 (CH₂, C20).

### Example 130: Tetrahydrofuran-3-yl 2-(4-ethoxyphenyl)thiazole-4-carboxylate (AM-3-187a)

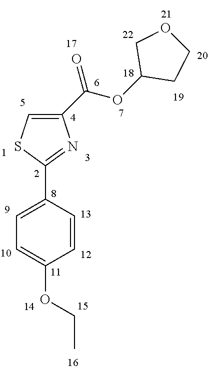 [SMILES: CCOc1ccc(-c2nc(C(=O)OC3CCOC3)cs2)cc1]

According to General Procedure C, 2-(4-ethoxyphenyl)thiazole-4-carboxylic acid (1.0 eq., 301 μmol, 75 mg) was esterified with 3-tetrahydrofuranol (1.5 eq, 451 μmol, 36 μL). The crude product was purified via column chromatography (30 mL SiO₂, eluent CH/EA/2.5:1).

Yield: 74 mg (221 μmol, 77%) colorless solid

C₁₆H₁₇NO₄S [319.38]

m.p.: 130° C.

R_{f}: 0.51 (CH/EA 1:1)

¹H-NMR (300 MHz, CDCl₃): δ 8.06 (s, 1H, H5), 7.93 (d, J=8.7 Hz, 2H, H9/13), 6.94 (d, J=8.7 Hz, 2H, H10/12), 5.57 (d, J=2.3 Hz, 1H, H18), 4.08 (q, J=6.9 Hz, 2H, H15), 4.05-3.97 (m, 3H, H20/22), 3.91 (m, 1H, H20), 2.41-2.15 (m, 2H, H19), 1.44 (t, J=6.9 Hz, 3H, H16).

¹³C-NMR (75.5 MHz, CDCl₃): δ 169.1 (C_{q}, C2), 161.3 (2C, C_{q}, C6/11), 147.5 (C_{q}, C4), 128.7 (2C, CH, C9/13), 126.7 (CH, C5), 125.6 (C_{q}, C8), 114.9 (2C, CH, C10/12), 76.0 (CH, C18), 73.2 (CH₂, C22), 67.3 (CH₂, C20), 63.8 (CH₂, C15), 33.0 (CH₂, C19), 14.9 (CH₃, C16).

### Example 131: Pentan-3-yl 2-(4-ethoxyphenyl)thiazole-4-carboxylate (AM-3-187b)

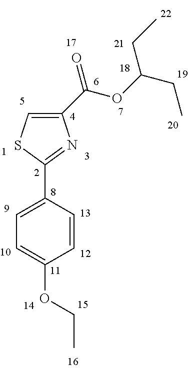 [SMILES: CCOc1ccc(-c2nc(C(=O)OC(CC)CC)cs2)cc1]

According to General Procedure C, 2-(4-ethoxyphenyl)thiazole-4-carboxylic acid (1.0 eq., 301 μmol, 75 mg) was esterified with 3-pentanol (1.5 eq, 451 μmol, 49 μL). The crude product was purified via column chromatography (30 mL SiO₂, eluent CH/EA/10:1).

Yield: 83 mg (260 μmol, 86%) colorless solid

C₁₇H₂₁NO₃S [319.42]

m.p.: 55° C.

R_{f}: 0.84 (CH/EA 1:1)

¹H-NMR (300 MHz, CDCl₃): δ 8.03 (s, 1H, H5), 7.95 (d, J=8.7 Hz, 2H, H9/13), 6.94 (d, J=8.7 Hz, 2H, H10/12), 5.10-4.99 (m, 1H, H18), 4.08 (q, J=6.9 Hz, 2H, H15), 1.79-1.65 (m, 4H, H19/21), 1.44 (t, J=6.9 Hz, 3H, H16), 0.96 (t, J=7.4 Hz, 6H, H20/21).

¹³C-NMR (75.5 MHz, CDCl₃): δ 168.8 (C_{q}, C2), 161.5 (C_{q}, C11), 161.2 (C_{q}, C6), 148.3 (C_{q}, C4), 128.7 (2C, CH, C9/13), 125.8 (C_{q}, C8), 125.8 (CH, C5), 114.9 (2C, CH, C10/12), 78.0 (CH, 18), 63.8 (CH₂, C15), 26.7 (2C, CH₂, C19/21), 14.9 (CH₃, C16), 9.9 (2C, CH₃, C20/22).

### Example 132: Phenyl 2-(4-ethoxyphenyl)thiazole-4-carboxylate (AM-3-188a)

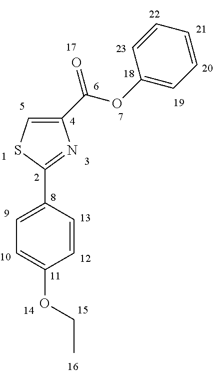 [SMILES: CCOc1ccc(-c2nc(C(=O)Oc3ccccc3)cs2)cc1]

According to General Procedure C, 2-(4-ethoxyphenyl)thiazole-4-carboxylic acid (1.0 eq., 301 μmol, 75 mg) was esterified with phenol (1.5 eq, 451 μmol, 42 mg). The crude product was purified via column chromatography (50 mL SiO₂, eluent CH/EA/10:1) and preparative HPLC (method B).

Yield: 66 mg (203 μmol, 67%) colorless solid

C₁₈H₁₅NO₃S [325.38]

m.p.: 100° C.

R_{f}: 0.32 (CH/EA 4:1)

¹H-NMR (300 MHz, DMSO-d₆): δ 8.75 (s, 1H, H5), 7.93 (d, J=8.6 Hz, 2H, H9/13), 7.47 (t, J=7.7 Hz, 2H, H20/22), 7.30 (t, J=9.1 Hz, 3H, H19/21/23), 7.05 (d, J=8.7 Hz, 2H, H10/12), 4.09 (q, J=6.9 Hz, 2H, H15), 1.33 (t, J=6.9 Hz, 3H, H16).

¹³C-NMR (75.5 MHz, DMSO-d₆): δ 168.0 (C_{q}, C2), 160.7 (C_{q}, C11), 159.2 (C_{q}, C6), 150.3 (C_{q}, C18), 145.7 (C_{q}, C4), 130.2 (CH, C5), 129.6 (2C, CH, C20/22), 128.2 (2C, CH, C9/13), 126.1 (CH, C21), 124.9 (C_{q}, C8), 121.9 (2C, CH, C19/23), 115.1 (2C, CH, C10/12), 63.5 (CH₂, C15), 14.5 (CH₃, C16).

### Example 133: Cyclohexyl 2-(4-ethoxyphenyl)thiazole-4-carboxylate (AM-3-188b)

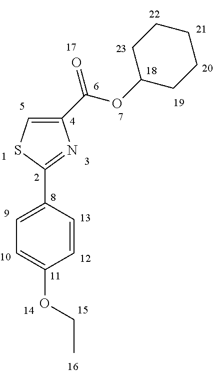 [SMILES: CCOc1ccc(-c2nc(C(=O)OC3CCCCC3)cs2)cc1]

According to General Procedure C, 2-(4-ethoxyphenyl)thiazole-4-carboxylic acid (1.0 eq., 301 μmol, 75 mg) was esterified with phenol (1.5 eq, 451 μmol, 48 μL). The crude product was purified via column chromatography (35 mL SiO₂, eluent CH/EA/10:1).

Yield: 89 mg (269 μmol, 89%) colorless solid

C₁₈H₂₁NO₃S [331.43]

m.p.: 128° C.

R_{f}: 0.38 (CH/EA 4:1)

¹H-NMR (300 MHz, CDCl₃): δ 8.03 (s, 1H, H5), 7.94 (d, J=8.7 Hz, 2H, H9/13), 6.94 (d, J=8.7 Hz, 2H, H10/12), 5.12-4.97 (m, 1H, H18), 4.08 (q, J=6.9 Hz, 2H, H15), 1.98 (s, 2H, H19/23), 1.78 (s, 2H, H20/22), 1.60 (m, 3H, H19/21/23), 1.45 (m, 5H, H18/20/22), 1.34-1.22 (m, 1H, H21).

¹³C-NMR (75.5 MHz, CDCl₃): δ 168.8 (C_{q}, C2), 161.2 (C_{q}, C11), 161.0 (C_{q}, C6), 148.4 (C_{q}, C4), 128.7 (2C, CH, C9/13), 125.9 (CH, C5), 125.8 (C_{q}, C8), 114.9 (2C, CH, C10/12), 73.9 (CH, C18), 63.8 (CH₂, C15), 31.8 (2C, CH₂, C19/23), 25.6 (CH₂, C21), 24.0 (2C, CH₂, C20/22), 14.9 (CH₃, C16).

### Example 134: (Tetrahydrofuran-2-yl)methyl 2-(4-ethoxyphenyl)thiazole-4-carboxylate (AM-3-190b)

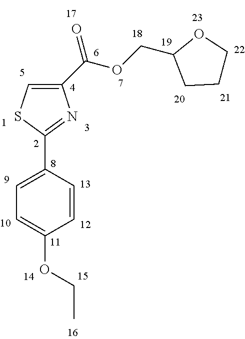 [SMILES: CCOc1ccc(-c2nc(C(=O)OCC3CCCO3)cs2)cc1]

According to general procedure C, 2-(4-ethoxyphenyl)thiazole-4-carboxylic acid (1.0 eq., 301 μmol, 75 mg) was esterified with tetrahydrofurfurylalcohol (1.5 eq, 451 μmol, 43 μL). The crude product was purified via column chromatography (35 mL SiO₂, eluent CH/EA 2.4:1).

Yield: 65 mg (195 μmol, 68%) colorless solid

C₁₇H₁₉NO₄S [333.40]

m.p.: 75° C.

R_{f}: 0.56 (CH/EA 1:1)

¹H-NMR (300 MHz, CDCl₃): δ 8.10 (s, 1H, H5), 7.93 (d, J=8.6 Hz, 2H, H9/13), 6.93 (d, J=8.6 Hz, 2H, H10/12), 4.41 (t, J=7.0 Hz, 1H, H18), 4.36-4.23 (m, 2H, H18/19), 4.08 (q, J=6.9 Hz, 2H, H15)), 3.94 (dd, J=14.6, 6.9 Hz, 1H, H22), 3.83 (dd, J=14.1, 7.3 Hz, 1H, H22), 2.14-1.86 (m, 3H, H10/21), 1.79-1.65 (m, J=10.5, 6.9 Hz, 1H, H20), 1.43 (t, J=6.9 Hz, 3H, H16).

¹³C-NMR (75.5 MHz, CDCl₃): δ 168.9 (C_{q}, C2), 161.5 (C_{q}, C11), 161.2 (C_{q}, C6), 147.5 (C_{q}, C4), 128.7 (2C, CH, C9/13), 126.7 (CH, C5), 125.7 (C_{q}, C8), 114.9 (2C, CH, C10/12), 76.6 (CH₂, C22), 68.6 (CH₂, C18), 67.3 (CH₂, C15), 63.8 (CH₂, C15), 28.3 (CH₂, C20), 25.8 (CH₂, C21), 14.9 (CH₃, C16).

### Example 135: 1,3-Difluoropropan-2-yl 2-(4-ethoxyphenyl)thiazole-4-carboxylate (AM-3-210)

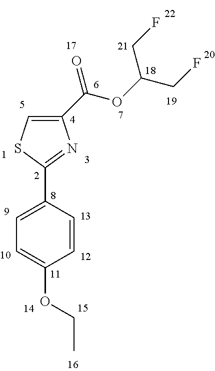 [SMILES: CCOc1ccc(-c2nc(C(=O)OC(CF)CF)cs2)cc1]

According to general procedure C, 2-(4-ethoxyphenyl)thiazole-4-carboxylic acid (1.0 eq., 297 μmol, 74 mg) was esterified with 1,3-difluoropropan-2-ol (1.5 eq, 445 μmol, 35 μL). The crude product was purified via column chromatography (30 mL SiO₂, eluent CH/EA 6:1).

Yield: 88 mg (269 μmol, 90%) colorless solid

C₁₅H₁₅F₂NO₃S [327.35]

m.p.: 120° C.

R_{f}: 0.68 (CH/EA 1:1)

¹H-NMR (300 MHz, CDCl₃): δ 8.15 (s, 1H, H5), 7.94 (d, J=8.7 Hz, 2H, H9/13), 6.95 (d, J=8.7 Hz, 2H, H10/12), 5.61-5.35 (m, 1H, H18), 4.83 (d, J=4.4 Hz, 2H, H19), 4.67 (d, J=3.7 Hz, 2H, H21), 4.09 (q, J=6.9 Hz, 2H, H15), 1.44 (t, J=7.0 Hz, 3H, H16).

¹³C-NMR (75.5 MHz, CDCl₃): δ 169.3 (C_{q}, C2), 161.3 (C_{q}, C11), 160.5 (C_{q}, C6), 146.6 (C_{q}, C4), 128.7 (2C, CH, C9/13), 127.6 (CH, C5), 125.5 (C_{q}, C8), 115.0 (2C, CH, C10/12), 81.5 (d, CH₂, C21), 79.2 (d, CH₂, C19), 71.3 (t, CH, C18), 63.9 (CH₂, C15), 14.9 (CH₃, C16).

### Example 13 Cyclopropylmethyl 2-(4-ethoxyphenyl)thiazole-4-carboxylate (AM-3-213a)

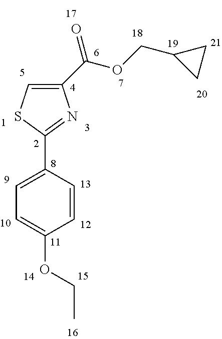 [SMILES: CCOc1ccc(-c2nc(C(=O)OCC3CC3)cs2)cc1]

According to general procedure C, 2-(4-ethoxyphenyl)thiazole-4-carboxylic acid (1.0 eq., 301 μmol, 75 mg) was esterified with 1,3-difluoropropan-2-ol (1.5 eq, 451 μmol, 37 μL). The crude product was purified via column chromatography (40 mL SiO₂, eluent CH/EA 12:1).

Yield: 65 mg (214 μmol, 71%) colorless solid

C₁₆H₁₇NO₃S [303.38]

m.p.: 110° C.

R_{f}: 0.81 (CH/EA 1:1)

¹H-NMR (300 MHz, CDCl₃): δ 8.10 (s, 1H, H5), 7.94 (d, J=8.8 Hz, 2H, H9/13), 6.94 (d, J=8.8 Hz, 2H, H10/12), 4.21 (d, J=7.3 Hz, 2H, H18), 4.09 (q, J=6.9 Hz, 2H, H15), 1.44 (t, J=7.0 Hz, 3H, H16), 1.35-1.20 (m, 1H, H19), 0.70-0.52 (m, 2H, H20/21), 0.52-0.29 (m, 2H, H20/21).

¹³C-NMR (75.5 MHz, CDCl₃): δ 169.0 (C_{q}, C2), 161.8 (C_{q}, C11), 161.2 (C_{q}, C6), 148.0 (C_{q}, C4), 128.7 (2C, CH, C9/13), 126.4 (CH, C5), 125.8 (C_{q}, C8), 114.9 (2C, CH, C10/12), 70.3 (CH₂, C18), 63.8 (CH₂, C15), 14.9 (CH₃, C16), 10.1 (CH, C19), 3.6 (2C, CH₂, C20/21).

### Example 137: 1,1,1-Trifluoro-3-methylbutan-2-yl 2-(4-ethoxyphenyl)thiazole-4-carboxylate (AM-3-220a)

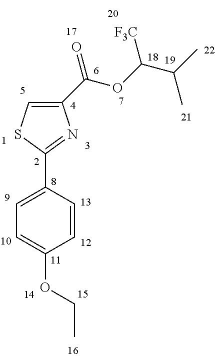 [SMILES: CCOc1ccc(-c2nc(C(=O)OC(C(C)C)C(F)(F)F)cs2)cc1]

According to general procedure C, 2-(4-ethoxyphenyl)thiazole-4-carboxylic acid (1.0 eq., 401 μmol, 100 mg) was esterified with 1,1,1,-trifluoro-3-methylbutan-2-ol (1.5 eq, 602 μmol, 75 μL). The crude product was purified via column chromatography (50 mL SiO₂, eluent CH/EA 12:1).

Yield: 93 mg (249 μmol, 62%) colorless solid

C₁₇H₁₈F₃NO₃S [373.39]

R_{f}: 0.47 (CH/EA 4:1)

¹H-NMR (300 MHz, CDCl₃): δ 8.15 (s, 1H, H5), 7.95 (d, J=8.7 Hz, 2H, H9/13), 6.95 (d, J=8.7 Hz, 2H, H10/12), 5.42 (dt, J=14.1, 7.2 Hz, 1H, H18), 4.09 (q, J=6.9 Hz, 2H, H15), 2.31 (td, J=13.1, 6.5 Hz, 1H, H19), 1.44 (t, J=6.9 Hz, 3H, H16), 1.10 (d, J=6.5 Hz, 6H, H21/22).

¹³C-NMR (75.5 MHz, CDCl₃): δ 169.4 (C_{q}, C2), 161.4 (C_{q}, C11), 159.8 (C_{q}, C6), 146.2 (C_{q}, C4), 128.7 (2C, CH, C9/13), 127.6 (CH, C5), 125.5 (C_{q}, C8), 115.0 (2C, CH, C10/12), 74.1 (q, CH, C18), 63.9 (CH₂, C15), 28.2 (CH, C19), 19.2 (CH₃, C21), 17.6 (CH₃, C22), 14.9 (CH₃, C16).

### Example 138: 3-(Methylamino)propyl 2-(4-ethoxyphenyl)thiazole-4-carboxylate hydrochloride (AM-3-238)

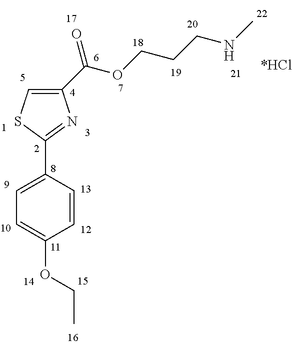 [SMILES: CCOc1ccc(-c2nc(C(=O)OCCCNC)cs2)cc1]

According to general procedure C, 2-(4-ethoxyphenyl)thiazole-4-carboxylic acid (1.0 eq., 482 μmol, 120 mg) was esterified with tert-butyl (3-hydroxypropylxmethyl)carbamate (1.5 eq, 722 μmol, 102 μL). The crude product was purified via column chromatography (60 mL SiO₂, eluent CH/EA 3:1). An inert Schienk flask was charged with the Boc-protected product and 1.0 mL HCl 4 M in dioxane was added. The reaction was stirred at RT overnight, when a colorless precipitate formed. The precipitate was collected by filtration and washed with EA. No further purification was necessary.

Yield: 101 mg (283 μmol, 59%) colorless solid

C₁₈H₂₀N₂O₃S*HCl [356.87]

¹H-NMR (300 MHz, DMSO-d₆): δ 8.94 (bs, 2H, NH₂), 8.55 (s, 1H, H5), 7.88 (d, J=8.6 Hz, 2H, H9/13), 7.04 (d, J=8.7 Hz, 2H, H10/12), 4.33 (t, J=5.9 Hz, 2H, H18), 4.08 (t, J=6.8 Hz, 2H, H15), 3.01 (s, 2H, H, H20), 2.53 (s, 3H, H22), 2.13-1.98 (m, 2H, H19), 1.32 (t, J=6.9 Hz, 3H, H16).

¹³C-NMR (75.5 MHz, DMSO-d₆): δ 167.7 (C_{q}, C2), 160.6 (C_{q}, C11), 160.5 (C_{q}, C6), 146.5 (C_{q}, C4), 128.6 (CH, C5), 128.1 (2C, CH, C9/13), 124.9 (C_{q}, C8), 115.1 (2C, CH, C10/12), 63.4 (CH₂, C15), 62.0 (CH₂, C18), 45.5 (CH₂, C20), 32.4 (CH₃, C22), 24.9 (CH₂, C19), 14.5 (CH₃, C16).

### Example 139: (1S,2R)-2-Methylcyclopentyl 2-(4-ethoxyphenyl)thiazole-4-carboxylate (AM-3-239e)

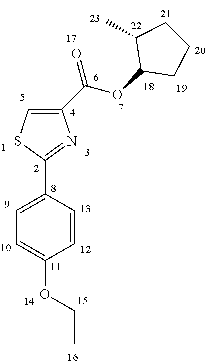

According to general procedure C, 2-(4-ethoxyphenyl)thiazole-4-carboxylic acid (1.0 eq., 301 μmol, 75 mg) was esterified with trans-2-methylcyclopentan-2-ol (1.5 eq, 451 μmol, 49 μL). The crude product was purified via column chromatography (50 mL SiO₂, eluent CH/EA 7.5:1) and preparative HPLC (method E).

Yield: 55 mg (166 μmol, 55%) colorless solid

C₁₈H₂₁NO₃S [331.43]

m.p.: 50° C.

R_{f}: 0.84 (CH/EA 1:1)

¹H-NMR (300 MHz, CDCl₃): δ 8.01 (s, 1H, H5), 7.93 (d, J=8.7 Hz, 2H, H9/13), 6.93 (d, J=8.8 Hz, 2H, H10/12), 5.06-4.87 (m, 1H, H18), 4.07 (q, J=6.9 Hz, 2H, H15), 2.29-2.05 (m, 2H, H22, H21), 2.05-1.91 (m, 1H, H19), 1.85-1.64 (m, 3H, H20/21), 1.43 (t, J=7.0 Hz, 3H, H16), 1.27 (dt, J=12.5, 7.6 Hz, 1H, H19), 1.07 (d, J=6.9 Hz, 3H, H23).

¹³C-NMR (75.5 MHz, CDCl₃): δ 168.8 (C_{q}, C2), 161.6 (C_{q}, C11), 161.1 (C_{q}, C6), 148.3 (C_{q}, C4), 128.6 (2C, CH, C9/13), 125.9 (CH, C5), 125.8 (C_{q}, C8), 114.8 (2C, CH, C10/12), 83.8 (CH, C18), 63.8 (CH₂, C15), 40.2 (CH, C11), 32.1 (CH₂, C19), 31.6 (CH₂, C13), 22.7 (CH₂, C20), 18.4 (CH₃, C23), 14.8 (CH₃, C16).

### Example 140: 2,3-Dihydro-1H-Inden-1-yl 2-(4-ethoxyphenyl)thiazole-4-carboxylate (AM-3-240b)

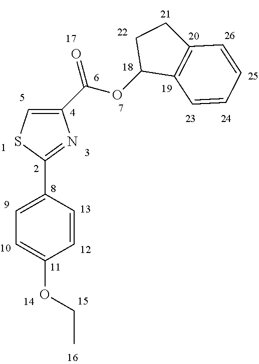 [SMILES: CCOc1ccc(-c2nc(C(=O)OC3CCc4ccccc43)cs2)cc1]

According to general procedure C, 2-(4-ethoxyphenyl)thiazole-4-carboxylic acid (1.0 eq., 301 μmol, 75 mg) was esterified with 2,3-dihydro-1H-inden-1-ol (1.5 eq, 451 μmol, 61 mg). The crude product was purified via column chromatography (50 mL SiO₂, eluent CH/EA 9:1).

Yield: 95 mg (266 μmol, 86%) colorless solid

C₂₁H₁₉NO₃S [365.45]

m.p.: 90° C.

R_{f}: 0.38 (CH/EA 4:1)

¹H-NMR (300 MHz, CD₃CN): δ 8.12 (s, 1H, H5), 7.84 (d, J=8.8 Hz, 2H, H9/13), 7.47 (d, J=7.3 Hz, 1H, H23), 7.38-7.16 (m, 3H, H24/25/26), 6.96 (d, J=8.8 Hz, 2H, H10/12), 6.39 (dd, J=6.8, 3.6 Hz, 1H, H18), 4.06 (q, J=6.9 Hz, 2H, H15), 3.21-3.05 (m, 1H, H22), 2.98-2.85 (m, 1H, H22), 2.64-2.48 (m, 1H, H21), 2.28-2.14 (m, 1H, H21), 1.35 (t, J=7.0 Hz, 3H, H16).

¹³C-NMR (75.5 MHz, CD₃CN): δ 169.3 (C_{q}, C2), 162.1 (C_{q}, C11), 162.1 (C_{q}, C6), 148.6 (C_{q}, C4), 145.8 (C_{q}, C20), 142.0 (C_{q}, C19), 130.1 (CH, C26), 129.1 (2C, CH, C9/13), 128.2 (CH, C25), 127.67, 126.6 (CH, C23), 126.5 (C_{q}, C8), 125.9 (CH, C24), 115.9 (2C, CH, C10/12), 80.1 (CH, C18), 64.7 (CH₂, C15), 33.0 (CH₂, C21), 30.8 (CH₂, C22), 15.0 (CH₃, C16).

### Example 141: Benzo[d][1,3]dixol-5-ylmethyl 2-(4-ethoxyphenyl)thiazole-4-carboxylate (AM-3-241a)

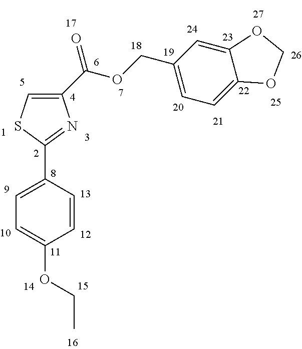 [SMILES: CCOc1ccc(-c2nc(C(=O)OCc3ccc4c(c3)OCO4)cs2)cc1]

According to general procedure C, 2-(4-ethoxyphenyl)thiazole-4-carboxylic acid (1.0 eq., 321 μmol, 80 mg) was esterified benzo[d][1,3]dioxol-5-ylmethanol (1.5 eq, 481 μmol, 73 mg). The crude product was purified via column chromatography (50 mL SiO₂, eluent CH/EA 6:1).

Yield: 78 mg (203 μmol, 63%) colorless solid

C₂₀H₁₇NO₅S [383.42]

m.p.: 105° C.

R_{f}: 0.78 (CH/EA 1:1)

¹H-NMR (300 MHz, CDCl₃): δ 8.08 (s, 1H, H5), 7.93 (d, J=8.7 Hz, 2H, H9/13), 7.04-6.87 (m, 4H, H10/12/21/24), 6.80 (d, J=7.8 Hz, 1H, H20), 5.97 (s, 2H, H26), 5.31 (s, 2H, H18), 4.08 (q, J=6.9 Hz, 2H, H15), 1.44 (t, J=6.9 Hz, 3H, H16).

¹³C-NMR (75.5 MHz, CDCl₃): δ 169.0 (C_{q}, C2), 161.5 (C_{q}, C11), 161.2 (C_{q}, C6), 148.0 (C_{q}, C23), 147.9 (C_{q}, C4), 147.6 (C_{q}, C22), 129.7 (C_{q}, C19), 128.7 (2C, CH, C9/13), 126.7 (CH, C5), 125.7 (C_{q}, C8), 122.8 (CH, C24), 114.9 (2C, CH, C10/12), 109.5 (CH, C21), 108.4 (CH, C20), 101.3 (CH₂, C26), 67.1 (CH₂, C18), 63.8 (CH₂, C15), 14.9 (CH₃, C16).

### Example 142: 4-Acetoxybenzyl 2-(4-ethoxyphenyl)thiazole-4-carboxylate (AM-3-241b)

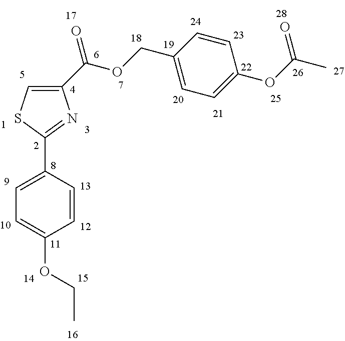 [SMILES: CCOc1ccc(-c2nc(C(=O)OCc3ccc(OC(C)=O)cc3)cs2)cc1]

According to general procedure C, 2-(4-ethoxyphenyl)thiazole-4-carboxylic acid (1.0 eq., 321 μmol, 80 mg) was esterified with 4-(hydroxymethyl)phenyl acetate (1.5 eq, 481 μmol, 80 mg). The crude product was purified three times via column chromatography (50 mL SiO₂, eluent CH/EA 5:1, 25 mL SiO₂, eluent toluene/EA 8:1, 20 mL SiO₂, eluent toluene/EA 10:1).

Yield: 46 mg (116 μmol, 36%) colorless solid

C₂₁H₁₉NO₅S [397.45]

m.p.: 102° C.

R_{f}: 0.71 (CH/EA 1:1)

¹H-NMR (300 MHz, CDCl₃): δ 8.09 (s, 1H, H5), 7.93 (d, J=8.7 Hz, 2H, H9/13), 7.50 (d, J=8.4 Hz, 2H, H21/23), 7.11 (d, J=8.4 Hz, 2H, H20/24), 6.94 (d, J=8.7 Hz, 2H, H10/12), 5.39 (s, 2H, H18), 4.09 (q, J=6.9 Hz, 2H, H15), 2.30 (s, J=14.6 Hz, 3H, H27), 1.44 (t, J=6.9 Hz, 3H, H16).

¹³C-NMR (75.5 MHz, CDCl₃): δ 169.5 (C_{q}, C2), 169.1 (C_{q}, C26), 161.4 (C_{q}, C11), 161.2 (C_{q}, C6), 150.8 (C_{q}, C22), 147.5 (C_{q}, C4), 133.5 (C_{q}, C19), 130.0 (2C, CH, C21/23), 128.7 (2C, CH, C9/13), 126.8 (CH, C5), 125.7 (C_{q}, C8), 121.9 (2C, CH, C20/24), 114.9 (2C, CH, C10/12), 66.4 CH₂, C18), 63.8 (CH₂, C15), 21.3 (CH₃, C27), 14.9 (CH₃, C16).

### Example 143: 3-Methylbut-2-en-1-yl 2-(4-ethoxyphenyl)thiazole-4-carboxylate (AM-4-254)

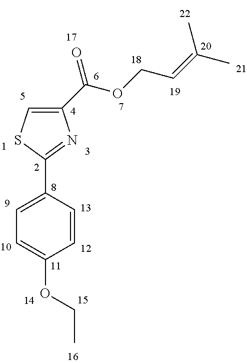 [SMILES: CCOc1ccc(-c2nc(C(=O)OCC=C(C)C)cs2)cc1]

According to general procedure C, 2-(4-ethoxyphenyl)thiazole-4-carboxylic acid (1.0 eq., 389 μmol, 97 mg) was esterified with prenol (1.5 eq, 584 μmol, 59 μL). The crude product was purified via column chromatography (50 mL SiO₂, eluent CH/EA 6:1).

Yield: 109 mg (343 μmol, 88%) colorless solid

C₁₇H₁₉NO₃S [317.40]

m.p.: 56° C.

R_{f}: 0.39 (CH/EA 4:1)

¹H-NMR (300 MHz, CDCl₃): δ 8.07 (s, 1H), 7.93 (d, J=8.8 Hz, 2H), 6.93 (d, J=8.8 Hz, 2H), 5.49 (t, J=7.1 Hz, 1H), 4.87 (d, J=7.1 Hz, 2H), 4.08 (q, J=6.9 Hz, 2H), 1.78 (s, J=31.7 Hz, 6H), 1.44 (t, J=7.0 Hz, 3H).

¹³C-NMR (75.5 MHz, CDCl₃): δ 168.9, 161.7, 161.2, 147.99, 139.5, 128.7, 126.3, 125.8, 118.7, 114.9, 63.8, 62.4, 26.0, 18.3, 14.9.

### Example 144: 1-(Thiophen-2-yl)ethyl 2-(4-ethoxyphenyl)thiazole-4-carboxylate (AM-4-287)

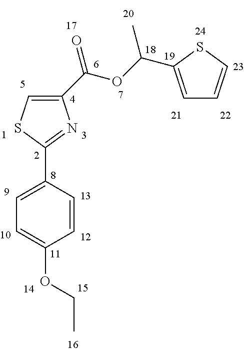 [SMILES: CCOc1ccc(-c2nc(C(=O)OC(C)c3cccs3)cs2)cc1]

According to general procedure C, 2-(4-ethoxyphenyl)thiazole-4-carboxylic acid (1.0 eq., 401 μmol, 100 mg) was esterified with 1-(thiophen-2-yl)ethan-1-ol (1.5 eq, 602 μmol, 124 mg). The crude product was purified via column chromatography (50 mL SiO₂, eluent CH/EA 9:1). The product was dissolved in acetonitrile, washed with pentane and evaporated to dryness.

Yield: 45 mg (125 μmol, 31%) colorless solid

C₁₈H₁₇NO₃S₂[359.46]

R_{f}: 0.36 (CH/EA 4:1)

¹H-NMR (300 MHz, CDCl₃): δ 8.03 (d, J=16.2 Hz, 1H), 7.93 (d, J=8.7 Hz, 2H), 7.29 (d, J=4.9 Hz, 1H), 7.16 (d, J=2.7 Hz, 1H), 7.08-6.82 (m, 3H), 6.46 (q, J=6.4 Hz, 1H), 4.08 (q, J=6.9 Hz, 2H), 1.81 (d, J=6.5 Hz, 3H), 1.44 (t, J=6.9 Hz, 3H).

¹³C-NMR (75.5 MHz, CDCl₃): δ 168.9, 161.2, 160.6, 147.7, 144.2, 128.6, 126.7, 126.6, 125.8, 125.7, 125.5, 114.84, 68.7, 63.8, 22.2, 14.8.

### Example 145: 1-Morpholinopropan-2-yl 2-(4-ethoxyphenyl)thiazole-4-carboxylate (AMU-10)

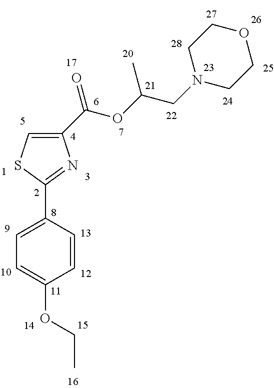 [SMILES: CCOc1ccc(-c2nc(C(=O)OC(C)CN3CCOCC3)cs2)cc1]

According to General Procedure C, 2-(4-ethoxyphenyl)thiazole-4-carboxylic acid (1.0 eq., 401 μmol, 100 mg) was esterified with 1-(4-morpholinyl)-2-propanol (1.5 eq, 602 μmol, 86 μL). The crude product was purified via column chromatography (110 mL SiO₂, eluent CH/EA 1:1).

Yield: 98 mg (261 μmol, 65%) brownish oil

C₁₉H₂₄N₂O₄S [376.15]

R_{f}: 0.30 (CH/EA 1:1)

¹H-NMR (300 MHz, CDCl₃): δ 8.04 (s, 1H), 7.93 (d, J=8.7 Hz, 2H), 6.94 (d, J=8.7 Hz, 2H), 5.47-5.31 (m, 1H), 4.09 (q, J=6.9 Hz, 2H), 3.67 (t, J=4.5 Hz, 4H), 2.71 (dd, J=13.1, 7.4 Hz, 1H), 2.64-2.42 (m, 5H), 1.49-1.34 (m, 6H).

¹³C-NMR (75.5 MHz, CDCl₃): δ 168.9, 161.2, 161.1, 148.2, 128.6, 126.1, 125.8, 114.90, 69.3, 67.2, 63.8, 63.5, 54.3, 18.7, 14.9.

### Example 146: 1,3-Bis(methylthio)propan-2-yl-2-(4-ethoxyphenyl)thiazole-4-carboxylate (AMU-14)

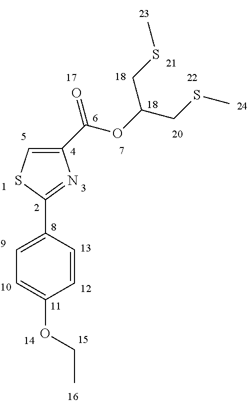 [SMILES: CCOc1ccc(-c2nc(C(=O)OC(CSC)CSC)cs2)cc1]

According to General Procedure C, 2-(4-ethoxyphenyl)thiazole-4-carboxylic acid (1.0 eq., 401 μmol, 100 mg) was esterified with 1,3-bis(methylthio)-2-propanol (1.5 eq, 602 μmol, 96 mg). The crude product was purified via column chromatography (55 mL SiO₂, eluent toluene/EA 30:1).

Yield: 90 mg (235 μmol, 59%) colorless solid

C₁₇H₂₁NO₃S₃ [383.07]

m.p.: 63-65° C.

R_{f}: 0.28 (toluene/EA 30:1)

¹H-NMR (300 MHz, CDCl₃): δ 8.08 (s, 1H), 7.93 (d, J=8.7 Hz, 2H), 6.94 (d, J=8.7 Hz, 2H), 5.58-5.05 (m, 1H), 4.09 (q, J=6.9 Hz, 2H), 3.10-2.77 (m, 4H), 2.23 (s, 6H), 1.44 (t, J=6.9 Hz, 3H).

¹³C-NMR (75.5 MHz, CDCl₃): δ 169.0, 161.3, 161.0, 147.4, 128.6, 126.8, 125.7, 114.93, 73.2, 63.8, 36.9, 16.6, 14.9.

### Example 147: Allyl 2-(4-ethoxyphenyl)thiazole-4-carboxylate (AMU-21)

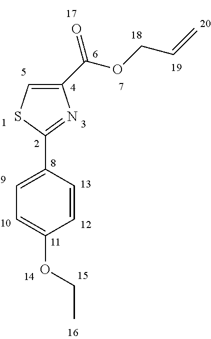 [SMILES: C=CCOC(=O)c1csc(-c2ccc(OCC)cc2)n1]

According to General Procedure C, 2-(4-ethoxyphenyl)thiazole-4-carboxylic acid (1.0 eq., 401 μmol, 100 mg) was esterified with allylalcohol (1.5 eq, 602 μmol, 41 μL). The crude product was purified via column chromatography (65 mL SiO₂, eluent CH/EA 15:1).

Yield: 31 mg (107 μmol, 26%) colorless solid

C₁₅H₁₅NO₃S [289.35]

m.p.: 102° C.

R_{f}: 0.21 (CH/EA 15:1)

¹H-NMR (300 MHz, CDCl₃): δ 8.10 (s, 1H), 7.94 (d, J=8.7 Hz, 2H), 6.94 (d, J=8.7 Hz, 2H), 6.13-6.00 (m, J=22.8, 11.0, 5.8 Hz, 1H), 5.37 (dd, J=37.5, 13.7 Hz, 2H), 4.87 (d, J=5.6 Hz, 2H), 4.09 (q, J=6.9 Hz, 2H), 1.44 (t, J=6.9 Hz, 3H).

¹³C-NMR (75.5 MHz, CDCl₃): 169.0, 161.3, 161.2, 147.6, 132.1, 128.7, 126.6, 125.7, 119.0, 114.9, 66.1, 63.8, 14.9.

### Example 148: But-3-en-2-yl 2-(4-ethoxyphenyl)thiazole-4-carboxylate (AMU-28)

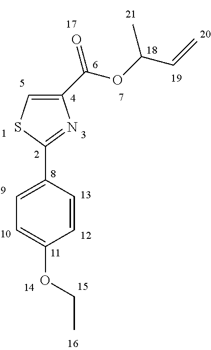 [SMILES: C=CC(C)OC(=O)c1csc(-c2ccc(OCC)cc2)n1]

According to General Procedure C, 2-(4-ethoxyphenyl)thiazole-4-carboxylic acid (1.0 eq., 401 μmol, 100 mg) was esterified with 2-buten-2-ol (1.5 eq, 602 μmol, 50 μL). The crude product was purified via column chromatography (60 mL SiO₂, eluent CH/EA 10:1).

Yield: 56 mg (185 μmol, 45%) colorless solid

C₁₆H₁₇NO₃S [303.38]

m.p.: 54° C.

R_{f}: 0.19 (CH/EA 10:1)

¹H-NMR (300 MHz, CDCl₃): δ 8.03 (d, J=20.0 Hz, 1H), 7.94 (d, J=8.7 Hz, 2H), 6.94 (d, J=8.7 Hz, 2H), 6.10-5.90 (m, J=16.7, 10.5, 5.9 Hz, 1H), 5.71-5.56 (m, 1H), 5.40-5.11 (m, 2H), 4.09 (q, J=6.9 Hz, 2H), 1.52-1.39 (m, J=15.4, 6.8 Hz, 6H).

¹³C-NMR (75.5 MHz, CDCl₃): δ 168.9, 161.2, 160.9, 148.1, 137.6, 128.7, 126.2, 125.8, 116.5, 114.9, 72.3, 63.8, 20.1, 14.9.

### Example 149: Prop-2-yn-1-yl 2-(4-ethoxyphenyl)thiazole-4-carboxylate (AMU-29)

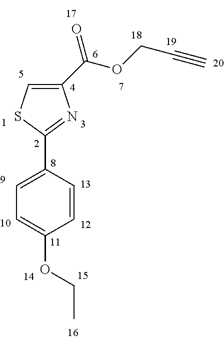 [SMILES: C#CCOC(=O)c1csc(-c2ccc(OCC)cc2)n1]

According to General Procedure C, 2-(4-ethoxyphenyl)thiazole-4-carboxylic acid (1.0 eq., 401 μmol, 100 mg) was esterified with propargyl alcohol (1.5 eq, 602 μmol, 40 μL). The crude product was purified via column chromatography (90 mL SiO₂, eluent CH/EA 10:1).

Yield: 42 mg (146 μmol, 31%) colorless solid

C₁₅H₁₃NO₃S [287.33]

m.p.: 83° C.

R_{f}: 0.16 (CH/EA 10:1)

¹H-NMR (300 MHz, Acetone-d₆): δ 8.41 (s, 1H), 7.97 (d, J=8.8 Hz, 2H), 7.07 (d, J=8.8 Hz, 2H), 4.99 (d, J=2.4 Hz, 2H), 4.23-4.08 (m, 2H), 3.13 (t, J=2.4 Hz, 1H), 1.41 (t, J=7.0 Hz, 3H).

¹³C-NMR (75.5 MHz, Acetone-d₆): δ 169.2, 162.2, 161.0, 147.7, 129.1, 128.69, 126.5, 115.9, 78.8, 76.7, 64.4, 52.9, 15.0.

### Example 150: tert-Butyl 2-(4-ethoxyphenyl)thiazole-4-carboxylate (TSch-61a)

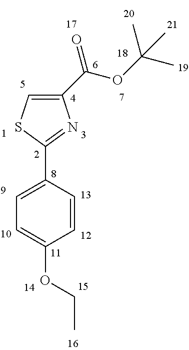 [SMILES: CCOc1ccc(-c2nc(C(=O)OC(C)(C)C)cs2)cc1]

According to General Procedure C, 2-(4-ethoxyphenyl)thiazole-4-carboxylic acid (1.0 eq., 401 μmol, 100 mg) was esterified with tert-butanol (1.5 eq, 602 μmol, 56 μL). The crude product was purified via column chromatography (50 mL SiO₂, eluent CH/EA 20:1).

Yield: 81 mg (265 μmol, 67%) colorless solid

C₁₆H₁₉NO₃S [305.39]

m.p.: 108-111° C.

R_{f}: 0.40 (CH/EA 5:1)

¹H-NMR (300 MHz, CDCl₃): δ (ppm) 7.95-7.93 (m, 3H), 6.95-6.92 (d, J=8.8 Hz, 2H), 4.14-4.05 (q, J=7.0 Hz, 2H), 1.62 (s, 9H), 1.46-1.42 (t, J=7.0 Hz, 3H).

¹³C-NMR (75.5 MHz, CDCl₃): δ (ppm) 168.6, 161.1, 160.8, 149.3, 128.6, 125.9, 125.4, 114.9, 82.1, 63.8, 28.4, 14.9.

### Example 151: Cyclopropyl 2-(4-ethoxyphenyl)thiazole-4-carboxylate (TSch-61d)

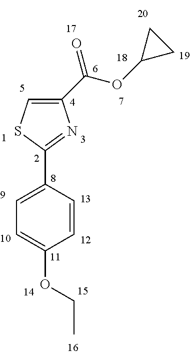 [SMILES: CCOc1ccc(-c2nc(C(=O)OC3CC3)cs2)cc1]

According to General Procedure C, 2-(4-ethoxyphenyl)thiazole-4-carboxylic acid (1.0 eq., 401 μmol, 100 mg) was esterified with cyclopropanol (1.5 eq, 602 μmol, 38 μL). The crude product was purified via column chromatography (40 mL SiO₂, eluent CH/EA 8:1).

Yield: 31 mg (107 μmol, 27%) colorless solid

C₁₅H₁₅NO₃S [289.35]

m.p.: 89-92° C.

R_{f}: 0.28 (CH/EA 5:1)

¹H-NMR (300 MHz, CDCl₃): δ (ppm) 8.04 (s, 1H), 7.93-7.90 (d, J=8.7 Hz, 2H), 6.94-6.91 (d, J=8.7 Hz, 2H), 4.39-4.34 (m, 1H), 4.11-4.04 (q, J=6.9 Hz, 2H), 1.45-1.40 (t, J=6.9 Hz, 3H), 0.88-0.79 (m, 4H).

¹³C-NMR (75.5 MHz, CDCl₃): δ (ppm) 169.0, 162.5, 161.2, 147.5, 128.6, 126.5, 125.6, 114.9, 63.8, 50.0, 14.8, 5.5.

### Example 152: Thiophen-2-ylmethyl 2-(4-ethoxyphenyl)thiazole-4-carboxylate (TSch-62a)

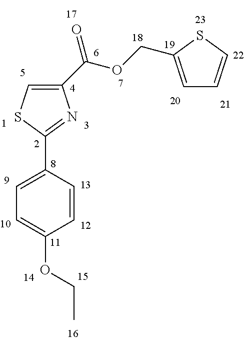 [SMILES: CCOc1ccc(-c2nc(C(=O)OCc3cccs3)cs2)cc1]

According to General Procedure C, 2-(4-ethoxyphenyl)thiazol 4-carboxylic acid (1.0 eq., 401 μmol, 100 mg) was esterified with 2-thiophenemethanol (1.5 eq, 602 μmol, 57 μL). The crude product was purified via column chromatography (50 mL SiO₂, eluent CH/EA 8:1).

Yield: 90 mg (260 μmol, 65%) yellowish solid

C₁₇H₁₅NO₃S₂ [345.43]

m.p.: 101-103° C.

R_{f}: 0.20 (CH/EA 8:1)

¹H-NMR (300 MHz, CDCl₃): δ (ppm) 8.09 (s, 1H), 7.94-7.91 (d, J=8.7 Hz, 2H), 7.35-7.33 (d, J=5.0 Hz, 1H), 7.21-7.20 (d, J=2.9 Hz, 1H), 7.02-7.00 (dd, ³J (H,H)=4.9 Hz, 1H), 6.95-6.92 (d, J=8.7 Hz, 2H), 5.56 (s, 2H), 4.11-4.05 (q, J=6.9 Hz, 2H), 1.46-1.41 (t, J=7.0 Hz, 3H).

¹³C-NMR (75.5 MHz, CDCl₃): δ (ppm) 169.0, 161.2, 161.2, 147.4, 137.7, 128.9, 128.7, 127.2, 127.0, 127.0, 125.6, 114.9, 63.8, 61.3, 14.9.

### Example 153: Furan-2-ylmethyl 2-(4-ethoxyphenyl)thiazole-4-carboxylate (TSch-62b)

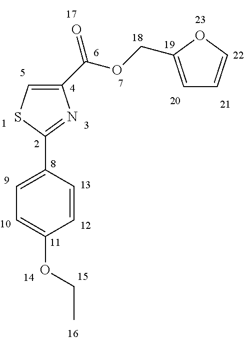 [SMILES: CCOc1ccc(-c2nc(C(=O)OCc3ccco3)cs2)cc1]

According to General Procedure C, 2-(4-ethoxyphenyl)thiazole-4-carboxylic acid (1.0 eq., 401 μmol, 100 mg) was esterified with furfuryl alcohol (1.5 eq, 602 μmol, 52 μL). The crude product was purified via column chromatography (50 mL SiO₂, eluent CH/EA 8:1).

Yield: 98 mg (298 μmol, 75%) yellowish solid

C₁₇H₁₅NO₄S [329.37]

m.p.: 72-74° C.

R_{f}: 0.46 (CH/EA 4:1)

¹H-NMR (300 MHz, CDCl₃): δ (ppm) 8.09 (s, 1H), 7.94-7.91 (d, J=8.8 Hz, 2H), 7.44 (d, J=0.9 Hz, 1H), 6.94-6.91 (d, J=8.8 Hz, 2H), 6.52-6.51 (d, J=3.0 Hz, 1H), 6.39-6.38 (dd, J=4.8 Hz, 1H), 5.35 (s, 2H), 4.11-4.04 (q, J=6.9 Hz, 2H), 1.46-1.41 (t, J=7.0 Hz, 3H).

¹³C-NMR (75.5 MHz, CDCl₃): δ (ppm) 169.0, 161.2, 161.2, 149.4, 147.3, 143.5, 128.7, 127.0, 125.6, 114.9, 111.4, 110.8, 63.8, 58.8, 14.9.

### Example 154: Isopentyl 2-(4-ethoxyphenyl)thiazole-4-carboxylate (TSch-62d)

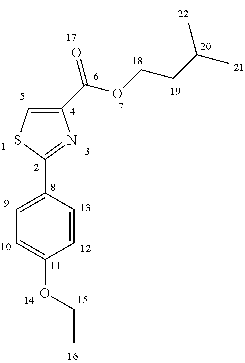 [SMILES: CCOc1ccc(-c2nc(C(=O)OCCC(C)C)cs2)cc1]

According to General Procedure C, 2-(4-ethoxyphenyl)thiazole-4-carboxylic acid (1.0 eq., 401 μmol, 100 mg) was esterified with isopentyl alcohol (1.5 eq, 602 μmol, 65 μL). The crude product was purified via column chromatography (50 mL SiO₂, eluent CH/EA 8:1).

Yield: 112 mg (351 μmol, 88%) colorless solid

C₁₇H₂₁NO₃S [319.42]

m.p.: 49-52° C.

R_{f}: 0.58 (CH/EA 4:1)

¹H-NMR (300 MHz, CDCl₃): δ (ppm) 8.05 (s, 1H), 7.95-7.92 (d, J=8.7 Hz, 2H), 6.95-6.92 (d, J=8.7 Hz, 2H), 4.42-4.38 (t, J=6.8 Hz, 2H), 4.13-4.05 (q, J=6.9 Hz, 2H), 1.81-1.74 (m, 1H), 1.72-1.68 (t, J=6.7 Hz, 2H), 1.46-1.42 (t, J=9.3 Hz, 3H), 0.98-0.96 (d, J=6.4 Hz, 6H).

¹³C-NMR (75.5 MHz, CDCl₃): δ (ppm) 168.9, 161.7, 161.2, 148.0, 128.7, 126.2, 125.7, 114.9, 64.2, 63.8, 37.5, 25.3, 22.7, 14.9.

### Example 155: (E)-Hex-2-en-1-yl 2-(4-ethoxyphenyl)thiazole-4-carboxylate (LS-8)

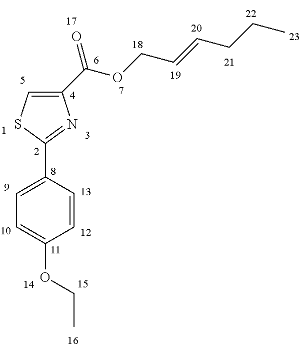 [SMILES: CCC/C=C/COC(=O)c1csc(-c2ccc(OCC)cc2)n1]

According to General Procedure C, 2-(4-ethoxyphenyl)thiazole-4-carboxylic acid (1.0 eq., 401 μmol, 100 mg) was esterified with trans-2-hexen-1-ol (1.5 eq, 602 μmol, 107 μL). The crude product was purified twice via column chromatography (60 mL SiO₂, eluent CH/EA 8:1 and 15 mL SiO₂, eluent toluene/EA 30:1).

Yield: 35 mg (106 μmol, 26%) colorless solid

C₁₈H₂₁NO₃S [331.43]

m.p.: 46-48° C.

R_{f}: 0.26 (toluene/EA 30:1)

¹H-NMR (300 MHz, CDCl₃): δ (ppm) 8.08 (s, 1H), 7.93 (d, J=8.8 Hz, 2H), 6.94 (d, J=8.8 Hz, 2H), 5.97-5.59 (m, 2H), 5.79-5.62 (m, 1H), 4.82 (d, J=6.4 Hz, 2H), 4.09 (q, J=6.9 Hz, 2H), 2.06 (dd, J=14.0, 6.8 Hz, 2H), 1.50-1.34 (m, 5H), 0.91 (t, J=7.3 Hz, 3H).

¹³C-NMR (75.5 MHz, CDCl₃): δ (ppm) 169.0, 161.5, 161.2, 147.9, 137.2, 128.7, 126.4, 125.7, 123.9, 114.89, 66.3, 63.8, 34.5, 22.2, 14.9, 13.8.

### Example 156: Hexyl 2-(4-ethoxyphenyl)thiazole-4-carboxylate (LS-9)

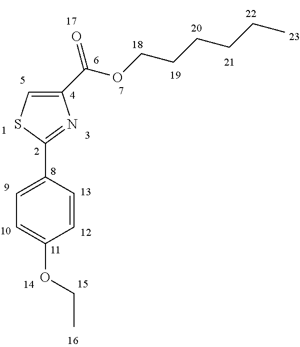 [SMILES: CCCCCCOC(=O)c1csc(-c2ccc(OCC)cc2)n1]

According to General Procedure C, 2-(4-ethoxyphenyl)thiazole-4-carboxylic acid (1.0 eq., 609 μmol, 150 mg) was esterified with 1-hexanol (1.5 eq, 903 μmol, 123 μL). The crude product was purified twice via column chromatography (70 mL SiO₂, eluent CH/EA 8:1 and 30 mL SiO₂, eluent toluene/EA 30:1).

Yield: 52 mg (156 μmol, 26%) colorless solid

C₁₈H₂₃NO₃S [333.45]

m.p.: 58-61° C.

R_{f}: 0.26 (toluene/EA 30:1)

¹H-NMR (300 MHz, CDCl₃): δ (ppm) 7.99 (s, J=18.6 Hz, 1H), 7.87 (d, J=8.8 Hz, 2H), 6.87 (d, J=8.8 Hz, 2H), 4.29 (t, J=6.8 Hz, 2H), 4.02 (q, J=6.9 Hz, 2H), 1.81-1.64 (m, 2H), 1.44-1.21 (m, 9H), 0.92-0.74 (m, J=6.7 Hz, 3H).

¹³C-NMR (75.5 MHz, CDCl₃): δ (ppm) 168.9, 161.7, 161.2, 148.0, 128.7, 126.2, 125.8, 114.9, 65.7, 63.8, 31.6, 28.8, 25.8, 22.7, 14.9, 14.1.

### Example 157: But-3-yn-2-yl 2-(4-ethoxyphenyl)thiazole-4-carboxylate (LS-11)

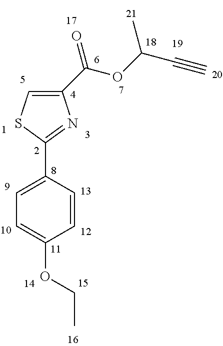 [SMILES: C#CC(C)OC(=O)c1csc(-c2ccc(OCC)cc2)n1]

According to General Procedure C, 2-(4-ethoxyphenyl)thiazole-4-carboxylic acid (1.0 eq., 401 μmol, 100 mg) was esterified with 3-butyn-2-ol (1.5 eq, 602 μmol, 47 μL). The crude product was purified via column chromatography (100 mL SiO₂, eluent CH/EA 8:1).

Yield: 113 mg (375 μmol, 94%) colorless solid

C₁₆H₁₅NO₃S [301.36]

m.p.: 86-88° C.

R_{f}: 0.41 (CH/EA 4:1)

¹H-NMR (300 MHz, CDCl₃): δ (ppm) 8.11 (s, 1H), 7.93 (d, J=8.8 Hz, 2H), 6.93 (d, J=8.7 Hz, 2H), 5.78-5.65 (m, 1H), 4.07 (q, J=6.9 Hz, 2H), 2.51 (d, J=2.0 Hz, 1H), 1.66 (d, J=6.7 Hz, 3H), 1.42 (t, J=7.0 Hz, 3H).

¹³C-NMR (75.5 MHz, CDCl₃): δ (ppm) 169.0, 161.21, 160.3, 147.2, 128.7, 127.0, 125.6, 114.9, 82.0, 73.6, 63.8, 61.2, 21.5, 14.8.

### Example 158: Benzyl 2-(4-ethoxyphenyl)thiazole-4-carboxylate (LS-12)

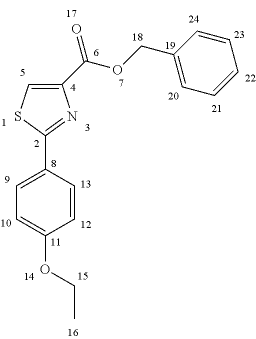 [SMILES: CCOc1ccc(-c2nc(C(=O)OCc3ccccc3)cs2)cc1]

According to General Procedure C, 2-(4-ethoxyphenyl)thiazole-4-carboxylic acid (1.0 eq., 401 μmol, 100 mg) was esterified with benzyl alcohol (1.5 eq, 602 μmol, 62 μL). The crude product was purified via column chromatography (100 mL SiO₂, eluent CH/EA 8:1).

Yield: 53 mg (156 μmol, 39%) colorless solid

C₁₉H₁₇NO₃S [339.41]

m.p.: 69-71° C.

R_{f}: 0.41 (CH/EA 4:1)

¹H-NMR (300 MHz, CDCl₃): δ (ppm) 8.09 (s, 1H), 7.94 (d, J=8.8 Hz, 2H), 7.41 (dt, J=14.9, 7.0 Hz, 5H), 6.94 (d, J=8.8 Hz, 2H), 5.42 (s, 2H), 4.09 (q, J=6.9 Hz, 2H), 1.44 (t, J=7.0 Hz, 3H).

¹³C-NMR (75.5 MHz, CDCl₃): δ (ppm) 169.0, 161.4, 161.2, 147.6, 136.0, 128.7, 128.7, 128.6, 128.5, 126.7, 125.7, 114.9, 67.1, 63.8, 14.9.

### Example 159: Pyridin-4-ylmethyl 2-(4-ethoxyphenyl)thiazole-4-carboxylate (LS-17)

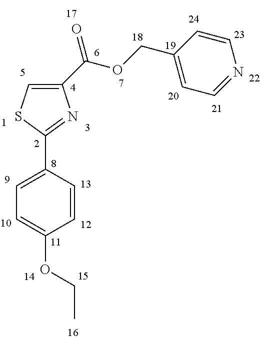 [SMILES: CCOc1ccc(-c2nc(C(=O)OCc3ccncc3)cs2)cc1]

According to General Procedure C, 2-(4-ethoxyphenyl)thiazole-4-carboxylic acid (1.0 eq., 401 μmol, 100 mg) was esterified with 4-pyridinmethanol (1.5 eq, 602 μmol, 66 mg). The crude product was purified via column chromatography (75 mL SiO₂, eluent CH/EA 1:1.5).

Yield: 88 mg (259 μmol, 65%) colorless solid

C₁₈H₁₆N₂O₃S [340.40]

m.p.: 124-128° C.

R_{f}: 0.16 (CH/EA 1:1)

¹H-NMR (300 MHz, CDCl₃): δ (ppm) 8.63 (d, J=5.5 Hz, 1H), 8.16 (s, 1H), 7.94 (d, J=8.8 Hz, 1H), 7.37 (d, J=5.0 Hz, 1H), 6.95 (d, J=8.7 Hz, 1H), 5.43 (s, 1H), 4.10 (q, J=6.9 Hz, 1H), 1.45 (t, J=7.0 Hz, 2H).

¹³C-NMR (75.5 MHz, CDCl₃): δ (ppm) 169.3, 161.4, 161.1, 150.2, 147.0, 144.9, 128.7, 127.3, 125.5, 122.2, 115.0, 65.0, 63.9, 14.9.

### Example 160: Pyridin-3-ylmethyl 2-(4-ethoxyphenyl)thiazole-4-carboxylate (LS-18)

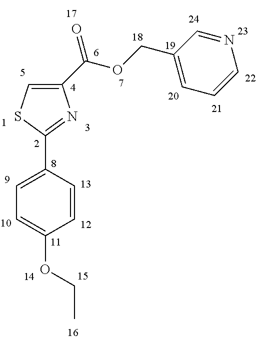 [SMILES: CCOc1ccc(-c2nc(C(=O)OCc3cccnc3)cs2)cc1]

According to General Procedure C, 2-(4-ethoxyphenyl)thiazole-4-carboxylic acid (1.0 eq., 401 μmol, 100 mg) was esterified with 3-pyridinmethanol (1.5 eq, 602 μmol, 58 μL). The crude product was purified via column chromatography (75 mL SiO₂, eluent CH/EA 1:1.5).

Yield: 101 mg (297 μmol, 74%) colorless solid

C₁₈H₁₆N₂O₃S [340.40]

m.p.: 100-103° C.

R_{f}: 0.16 (CH/EA 1:1)

¹H-NMR (300 MHz, CDCl₃): δ (ppm) 8.75 (s, 1H), 8.60 (d, J=3.5 Hz, 1H), 8.10 (s, 1H), 7.92 (d, J=8.8 Hz, 2H), 7.83 (d, J=7.8 Hz, 1H), 7.32 (dt, J=13.6, 6.9 Hz, 1H), 6.94 (d, J=8.8 Hz, 2H), 5.43 (s, 2H), 4.08 (q, J=6.9 Hz, 2H), 1.44 (t, J=7.0 Hz, 3H).

¹³C-NMR (75.5 MHz, CDCl₃): δ (ppm) 169.2, 161.3, 150.1, 149.9, 147.2, 136.5, 131.6, 128.7, 127.7, 125.6, 123.7, 115.0, 64.5, 63.8, 14.7.

### Example 161: Pyridin-2-ylmethyl 2(4-ethoxyphenyl)thiazole-4-carboxylate (LS-21)

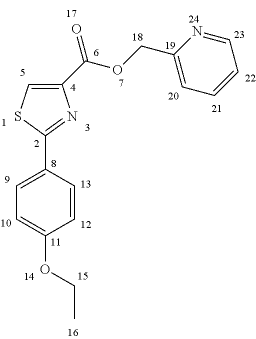 [SMILES: CCOc1ccc(-c2nc(C(=O)OCc3ccccn3)cs2)cc1]

According to General Procedure C, 2-(4-ethoxyphenyl)thiazole-4-carboxylic acid (1.0 eq., 401 μmol, 100 mg) was esterified with 2-pyridinmethanol (1.5 eq, 602 μmol, 58 μL). The crude product was purified via column chromatography (75 mL SiO₂, eluent CH/EA 1:1).

Yield: 69 mg (203 μmol, 51%) colorless solid

C₁₈H₁₆N₂O₃S [340.40]

m.p.: 123-127° C.

R_{f}: 0.32 (CH/EA 1:1)

¹H-NMR (300 MHz, CDCl₃): δ (ppm) 8.61 (d, J=3.9 Hz, 1H), 8.16 (s, 1H), 7.94 (d, J=8.8 Hz, 2H), 7.72 (td, J=7.7, 1.6 Hz, 1H), 7.48 (d, J=7.8 Hz, 1H), 7.31-7.17 (m, 1H), 6.94 (d, J=8.8 Hz, 2H), 5.53 (s, 2H), 4.08 (q, J=7.0 Hz, 2H), 1.44 (t, J=7.0 Hz, 3H).

¹³C-NMR (75.5 MHz, CDCl₃): δ (ppm) 169.1, 161.3, 155.9, 149.6, 147.3, 137.0, 128.7, 127.1, 125.6, 123.1, 122.1, 114.9, 67.5, 63.8, 14.9.

### Example 162: tert-Butyl 5-chloro-2(4-ethoxyphenyl)thiazole-4-carboxylate (AL-6)

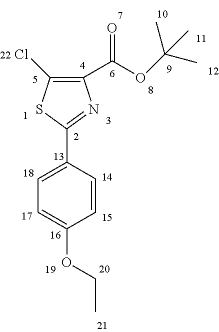 [SMILES: CCOc1ccc(-c2nc(C(=O)OC(C)(C)C)c(Cl)s2)cc1]

In a 50 mL round-bottom flask equipped with a magnetic stirring bar 700 mg tert-butyl 2-(4-ethoxyphenyl)thiazole-4-carboxylate (2.29 mmol, 1.0 eq.) and 368 mg N-chlorosuccinimide (2.75 mmol, 1.2 eq.) were dissolved in 20 mL MeCN (HPLC-grade). The flask was equipped with a reflux condenser and heated to 80° C. The reaction was monitored via TLC (CH/EA 4:1). After 3 h the conversion was incomplete and did not change during the subsequent 3 h. The reaction was cooled to RT and the solvent was evaporated in vacuo. The crude product was purified via column chromatography (CH/EA 10:1, 110 g SiO₂).

Yield: 364 mg (1.03 mmol, 47%, white solid)

C₁₆H₁₉NO₃S [339.83]

m.p.: 97-98° C.

R_{f}: 0.26 (CH/EA 12:1)

¹H-NMR (300 MHz, CDCl₃): δ (ppm) 7.83 (d, J=9.01 Hz, 2H, H-14/18), 6.94 (d, J=9.01 Hz, 2H, H-15/17), 4.07 (q, 2H, H-20), 1.63 (s, 9H, H-10/11/12), 1.43 (t, 3H, H-21)

¹³C-NMR (76 MHz, CDCl₃): δ (ppm) 164.30 (C_{q}, C-2), 161.39 (C_{q}, C-6), 160.12 (C_{q}, C-16), 143.24 (C_{q}, C-4), 131.67 (C_{q}, C-13), 128.25 (CH, C-14/18), 125.32 (C_{q}, C-5), 114.95 (CH, C-15/17), 82.91 (C_{q}, C-9), 63.86 (CH₂, C-20), 28.35 (CH₃, C-10/11/12), 14.85 (CH₃, C-21)

### Example 163: tert-Butyl 5-bromo-2-(4-ethoxyphenyl)thiazole-4-carboxylate (AM-4-272=RE-3)

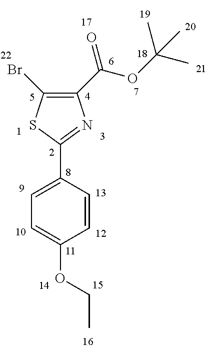 [SMILES: CCOc1ccc(-c2nc(C(=O)OC(C)(C)C)c(Br)s2)cc1]

In a 250 mL round-bottom flask equipped with a magnetic stirring bar 1.53 g tert-butyl 2-(4-ethoxyphenyl)thiazole-4-carboxylate (5.0 mmol, 1.0 eq.) and 1.079 g N-bromosuccinimide (6.06 mmol, 1.2 eq.) were dissolved in 35 mL MeCN (HPLC-grade). The flask was equipped with a reflux condenser and heated to 80° C. When full conversion was observed via TLC (CH/EA 4:1), the reaction was cooled down to RT and the solvent was evaporated in vacuo. The crude product was purified via column chromatography (500 mL SiO₂, eluent CH/EA 8:1).

Yield: 1.64 g (4.28 mmol, 86%, off-white solid)

C₁₆H₁₈BrNO₃S [384.29]

m.p.: 112-116° C.

R_{f}: 0.58 (CH/EA 4:1)

¹H-NMR (300 MHz, CDCl₃): δ (ppm) 7.83 (d, J=8.7 Hz, 2H), 6.92 (d, J=8.7 Hz, 2H), 4.08 (q, J=6.9 Hz, 2H), 1.64 (s, 9H), 1.43 (t, J=6.9 Hz, 3H).

¹³C-NMR (76 MHz, CDCl₃): δ (ppm) 167.6, 161.4, 160.4, 145.7, 128.3, 125.3, 115.0, 113.8, 83.0, 63.9, 28.4, 14.9.

### Example 164: Ethyl 5-bromo-2-(4-ethoxyphenyl)thiazole-4-carboxylate (OKO-06)

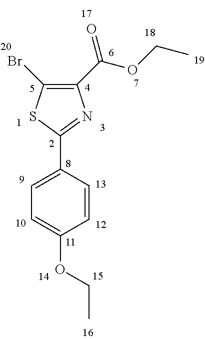 [SMILES: CCOC(=O)c1nc(-c2ccc(OCC)cc2)sc1Br]

In a 100 mL round-bottom flask equipped with a magnetic stirring bar ethyl 2-(4-ethoxyphenyl)thiazole-4-carboxylate (1.0 eq., 2.0 mmol, 556 mg) and N-bromosuccinimide (1.2 eq., 2.4 mmol, 425 mg) were dissolved in 10 mL MeCN (HPLC-grade). The flask was equipped with a reflux condenser and heated to 80° C. When full conversion was observed via TLC (CH/EA 4:1), the reaction was cooled down to RT and the solvent was evaporated in vacuo. The crude product was purified via column chromatography (500 mL SiO₂, eluent CH/EA 10:1).

Yield: 178 g (500 μmol, 25%) off-white solid

C₁₄H₁₄BrNO₃S [356.23]

R_{f}: 0.46 (CH/EA 4:1)

¹H-NMR (300 MHz, CDCl₃): δ (ppm) 7.82 (d, J=8.8 Hz, 2H), 6.93 (d, J=8.8 Hz, 2H), 4.45 (q, J=7.1 Hz, 2H), 4.08 (q, J=7.0 Hz, 2H), 1.52-1.36 (m, J=7.0, 2.3 Hz, 6H).

¹³C-NMR (76 MHz, CDCl₃): δ (ppm) 168.0, 161.5, 161.4, 144.3, 128.4, 125.2, 115.3, 115.0, 63.98, 61.9, 14.8, 14.4.

### Example 165: Ethyl 2-(4-ethoxyphenyl)-5-iodothiazole-4-carboxylate (OKO-31)

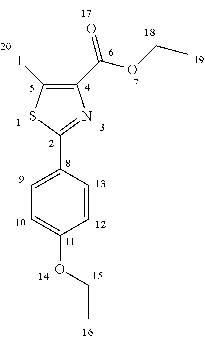 [SMILES: CCOC(=O)c1nc(-c2ccc(OCC)cc2)sc1I]

In a 100 mL round-bottom flask equipped with a magnetic stirring bar ethyl 2-(4-ethoxyphenyl)thiazole-4-carboxylate (1.0 eq., 1.81 mmol, 505 mg) and N-iodosuccinimide (2.1 eq., 3.83 mmol, 808 mg) were dissolved in 20 mL MeCN (HPLC-grade). The flask was equipped with a reflux condenser and heated to 80° C. After 17 h the reaction was cooled down to RT and the solvent was evaporated in vacuo. The crude product was purified via column chromatography (300 mL SiO₂, eluent CH/EA 8:1).

Yield: 410 g (1.02 mmol, 25%, with impurities) off-white solid

C₁₄H₁₄INO₃S [403.23]

m.p.: 88° C.

R_{f}: 0.46 (CH/EA 4:1)

¹H-NMR (300 MHz, CDCl₃): δ (ppm) 7.84 (d, J=8.7 Hz, 2H), 6.93 (d, J=8.7 Hz, 2H), 4.47 (q, J=7.1 Hz, 2H), 4.08 (q, J=6.9 Hz, 2H), 1.57-1.33 (m, J=14.8, 7.3 Hz, 6H).

¹³C-NMR (76 MHz, CDCl₃): δ (ppm) 173.1, 161.7, 161.4, 148.6, 128.5, 125.2, 115.0, 77.4, 63.8, 61.9, 14.8, 14.4.

### Example 166: tert-Butyl 2-(4-ethoxyphenyl)-5-(pyrrolidin-1-yl)thiazole-4-carboxylate (AL-10)

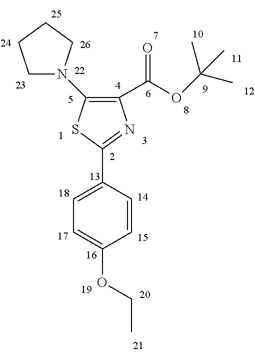 [SMILES: CCOc1ccc(-c2nc(C(=O)OC(C)(C)C)c(N3CCCC3)s2)cc1]

According to General Procedure D, 5-chloro-2-(4-ethoxyphenyl)thiazole-4-carboxylate (1.0 eq, 294 μmol, 100 mg) was coupled with pyrrolidine (2.1 eq, 609 μmol, 43 mg). The crude product was purified via column chromatography (20 g SiO₂, CH/EA 6:1).

Yield: 91 mg (243 μmol, 83%, white solid)

C₂₀H₂₆N₂O₃S [374.50]

m.p.: 149-150° C.

R_{f}: 0.61 (CH/EA 2:1)

¹H-NMR (300 MHz, CDCl₃): δ (ppm) 7.72 (d, J=9.01 Hz, 2H, H-14/18), 6.89 (d, J=9.01 Hz, 2H, H-15/17), 4.04 (q, 2H, H-20), 3.42 (t, 4H, H-23/26), 1.99 (t, 4H, H-24/25), 1.62 (s, 9H, H-10/11/12), 1.42 (t, 3H, H-21)

¹³C-NMR (76 MHz, CDCl₃): δ (ppm) 162.63 (C_{q}, C-2), 159.81 (C_{q}, C-6), 159.10 (C_{q}, C-16), 149.27 (C_{q}, C-4), 127.24 (C_{q}, C-13), 126.94 (CH, C-14/18), 124.39 (C_{q}, C-5), 114.65 (CH, C-15/17), 80.70 (C_{q}, C-9), 63.67 (CH₂, C-20), 55.40 (CH₂, C-23/26), 28.58 (CH₃, C-10/11/12), 26.37 (CH₂, C-23/26), 14.91 (CH₃, C-21)

### Example 167: tert-Butyl 2-(4-ethoxyphenyl)-5-(piperidin-1-yl)thiazole-4-carboxylate (LS-7)

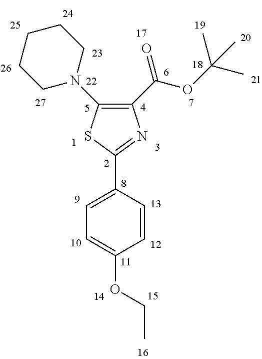 [SMILES: CCOc1ccc(-c2nc(C(=O)OC(C)(C)C)c(N3CCCCC3)s2)cc1]

According to General Procedure D, 5-chloro-2-(4-ethoxyphenyl)thiazole-4-carboxylate (1.0 eq, 294 μmol, 100 mg) was coupled with piperidine (2.1 eq, 609 μmol, 61 μL). The crude product was purified via column chromatography (100 mL SiO₂, CH/EA 8:1).

Yield: 27 mg (70 μmol, 24%, white solid)

C₂₁H₂₈N₂O₃S [388.53]

m.p.: 105-106° C.

R_{f}: 0.80 (CH/EA 2:1)

¹H-NMR (300 MHz, CDCl₃): δ (ppm) 7.78 (d, J=8.8 Hz, 2H), 6.88 (d, J=8.8 Hz, 2H), 4.05 (q, J=6.9 Hz, 2H), 3.21-3.03 (m, 4H), 1.81-1.71 (m, J=4.6 Hz, 4H), 1.63 (s, J=14.6 Hz, 11H), 1.41 (t, J=7.0 Hz, 3H).

¹³C-NMR (76 MHz, CDCl₃): δ (ppm) 162.4, 161.91, 160.2, 154.7, 131.0, 127.6, 126.7, 114.7, 81.2, 63.7, 56.1, 28.6, 25.7, 23.8, 14.9.

### Example 168: tert-Butyl 5-(dimethylamino)-2-(4-ethoxyphenyl)thiazole-4-carboxylate (LS-14)

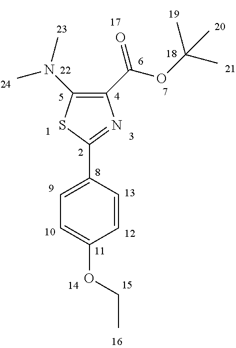 [SMILES: CCOc1ccc(-c2nc(C(=O)OC(C)(C)C)c(N(C)C)s2)cc1]

According to General Procedure D, 5-chloro-2-(4-ethoxyphenyl)thiazole-4-carboxylate (1.0 eq, 221 μmol, 75 mg) was coupled with dimethylamine (2.1 eq, 2.0 M in THF, 232 μL). The crude product was purified via column chromatography (30 ml SiO₂, CH/EA 8:1).

Yield: 50 mg (144 μmol, 65%, white solid)

C₁₈H₂₄N₂O₃S [348.46]

m.p.: 93-96° C.

R_{f}: 0.29 (CH/EA 8:1)

¹H-NMR (300 MHz, CDCl₃): δ (ppm) 7.77 (d, J=8.8 Hz, 2H), 6.89 (d, J=8.7 Hz, 2H), 4.06 (dd, J=13.9, 6.9 Hz, 2H), 3.01 (s, 6H), 1.63 (s, 9H), 1.42 (t, J=7.0 Hz, 3H).

¹³C-NMR (76 MHz, CDCl₃): δ (ppm) 163.1, 162.0, 160.2, 152.8, 128.7, 127.5, 126.7, 114.7, 81.2, 63.7, 46.8, 28.5, 14.9.

### Example 169: tert-Butyl 2-(4-ethoxyphenyl)-5-(1-hydroxyethyl)thiazole-4-carboxylate (RE-09)

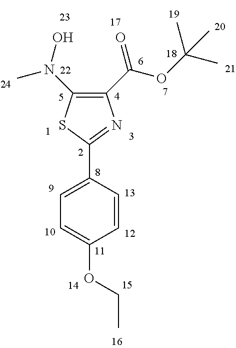 [SMILES: CCOc1ccc(-c2nc(C(=O)OC(C)(C)C)c(N(C)O)s2)cc1]

An inert 10 mL Schienk flask equipped with magnetic stirring bar was charged with tert-butyl 5-bromo-2-(4-ethoxyphenyl)thiazole-4-carboxylate (1.0 eq, 520 μmol, 200 mg) and cooled down to −15° C. using an ice/NaCl bath. iPrMgCl*LiCl (1.3 M in THF, 1.1 eq. 420 μL) was added and the reaction mixture turned dark red immediately. Acetaldehyde (1 M in THF, 1.2 eq., 624 μL) and CuCN*2LiCl (1 M in THF, 1 drop) was added. The reaction was allowed to warm to RT. After 2 h full conversion was observed via TLC (CH/EA 4:1). The reaction was quenched via the addition of HCl (1 M, 4 ml) and extracted with DCM (3×10 mL). The combined organic phase was dried over Na₂SO₄, filtered and the solvent was removed under reduced pressure. The crude product was purified via column chromatography (50 mL SiO₂, eluent CH/EA 4:1).

Yield: 132 mg (378 μmol, 73%) white solid

C₁₈H₂₃NO₄S [349.45]

m.p.: 155-158° C.

R_{f}: 0.24 (CH/EA 4:1)

¹H-NMR (300 MHz, CDCl₃): δ (ppm) 7.77 (d, J=8.8 Hz, 2H), 6.89 (d, J=8.7 Hz, 2H), 4.06 (q, J=6.9 Hz, 2H), 3.01 (s, 6H), 1.63 (s, 9H), 1.42 (t, J=7.0 Hz, 3H).

¹³C-NMR (75.5 MHz, CDCl₃): δ (ppm) 164.6, 162.4, 160.9, 152.8, 142.5, 128.2, 125.8, 114.7, 82.9, 63.8, 63.7, 28.2, 24.2, 14.7.

### Example 170: tert-Butyl 2-(4-ethoxyphenyl)-5-(1-hydroxy-2-methylpropyl)thiazole-4-carboxylate (RE-13a)

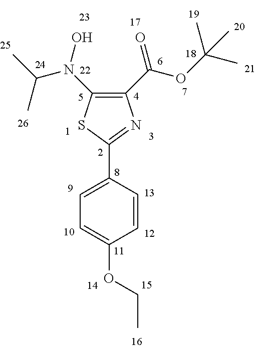 [SMILES: CCOc1ccc(-c2nc(C(=O)OC(C)(C)C)c(N(O)C(C)C)s2)cc1]

According to the synthesis of RE-09, tert-butyl 5-bromo-2-(4-ethoxyphenyl)thiazole-4-carboxylate (1.0 eq, 520 μmol, 200 mg) was converted to the corresponding Grignard reagent and subsequently treated with isobutyraldehyde (1 M in THF, 1.1 eq., 574 μL). The crude product was purified via column chromatography (50 mL SiO₂, eluent CH/EA 4:1).

Yield: 154 mg (408 μmol, 79%) orange solid

C₂₀H₂₇NO₄S [377.50]

m.p.: 116-120° C.

R_{f}: 0.20 (CH/EA 4:1)

¹H-NMR (300 MHz, CDCl₃): δ (ppm) 7.80 (d, J=8.6 Hz, 2H), 6.85 (d, J=8.6 Hz, 2H), 5.11 (d, J=6.7 Hz, 1H), 4.01 (dd, J=13.9, 6.9 Hz, 2H), 2.01 (td, J=13.3, 6.7 Hz, 1H), 1.57 (s, 9H), 1.36 (t, J=6.9 Hz, 3H), 1.00 (d, J=6.6 Hz, 3H), 0.88 (d, J=6.7 Hz, 3H).

¹³C-NMR (75.5 MHz, CDCl₃): δ (ppm) 165.2, 162.4, 161.0, 150.8, 142.8, 128.3, 125.9, 114.8, 82.8, 73.21, 63.4, 35.7, 28.4, 19.7, 18.1, 14.9.

### Example 171: tert-Butyl 5-acetyl-2-(4-ethoxyphenyl)thiazole-4-carboxylate (RE-10)

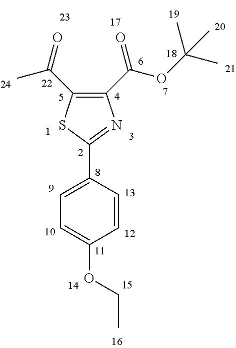 [SMILES: CCOc1ccc(-c2nc(C(=O)OC(C)(C)C)c(C(C)=O)s2)cc1]

An inert 25 mL round bottom flask equipped with magnetic stirring bar and Schienk adapter was charged with RE-09 (1.0 eq, 213 μM, 75 mg) and dissolved in anhydrous DCM. The reaction mixture was cooled down to 0° C. using and ice bath and Dess Martin-Periodinan (DMP) (1.5 eq. 320 μmol, 136 mg) was added. When full conversion was observed via TLC (CH/EA 4:1), the reaction was quenched via the addition of Na₂S₂O₃ (1 M. 5 mL) and sat. NaHCO₃ (5 mL). The aqueous phase was extracted with DCM (3×10 mL) and the combined organic phase was dried over Na₂SO₄, filtered and evaporated to dryness. The crude product was purified via column chromatography (25 mL SiO₂, eluent CH/EA 5:1).

Yield: 70 mg (202 μmol, 95%) orange solid

C₁₈H₂₁NO₄S [347.43]

m.p.: 89-95° C.

R_{f}: 0.38 (CH/EA 4:1)

¹H-NMR (300 MHz, CDCl₃): δ (ppm) 7.85 (d, J=8.8 Hz, 2H), 6.87 (d, J=8.8 Hz, 2H), 4.02 (q, J=6.9 Hz, 2H), 2.53 (s, 3H), 1.58 (s, J=8.8 Hz, 9H), 1.37 (t, J=7.0 Hz, 3H).

¹³C-NMR (75.5 MHz, CDCl₃): δ (ppm) 190.7, 171.1, 162.8, 162.0, 149.8, 137.1, 129.0, 125.2, 115.0, 84.1, 63.9, 29.9, 28.2, 14.8.

### Example 172: Ethyl 5-acetyl-2-(4-ethoxyphenyl)thiazole-4-carboxylate (RE-18)

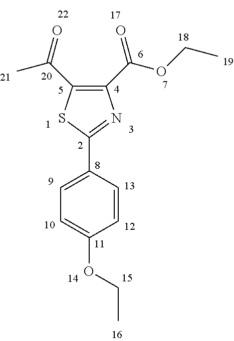 [SMILES: CCOC(=O)c1nc(-c2ccc(OCC)cc2)sc1C(C)=O]

An inert 50 mL round bottom flask equipped with magnetic stirring bar and Schlenk adapter was charged with RE-10 (1.0 eq., 164 μmol, 57 mg) and dissolved in 5.0 mL dry DCM. Et₃SiH (2.5 eq., 410 μmol, 65 μL) and TFA (12 eq., 1.1 mmol, 160 μL) were added successively. The suspension was stirred at 40° C. When full conversion was observed via TLC (CH/EA 1:2) the reaction mixture was cooled down to RT and the solvent was removed under reduced pressure. The solid residue was in 5 mL EtOH and 5 drops H₂SO₄ were added. The reaction was stirred at reflux until full conversion was observed via TLC (CH/EA 3:4). The solvent was again removed under reduced pressure and the residue was taken up in EA (20 mL) and washed with NaHCO₃ sat. (2×20 mL). The organic layer was dried over Na₂SO₄, filtered and the solvent was removed under reduced pressure. The crude product was purified via column chromatography (100 mL SiO₂, eluent CH/EA 5:4).

Yield: 20 mg (63 μmol, 38%) off-white solid

C₁₆H₁₇NO₄S [319.38]

m.p.: 78° C.

R_{f}: 0.76 (CH/EA 4:3)

¹H-NMR (300 MHz, CDCl₃): δ (ppm) 7.85 (d, J=8.8 Hz, 2H), 6.87 (d, J=8.8 Hz, 2H), 4.02 (q, J=6.9 Hz, 2H), 2.53 (s, 3H), 1.58 (s, J=8.8 Hz, 9H), 1.37 (t, J=7.0 Hz, 3H).

¹³C-NMR (75.5 MHz, CDCl₃): δ (ppm) 190.7, 171.1, 162.8, 162.0, 149.8, 137.1, 129.0, 125.2, 115.0, 84.1, 63.9, 29.9, 28.2, 14.8.

### Example 173: tert-Butyl 2-(4-ethoxyphenyl)-5-isobutyrylthiazole-4-carboxylate (RE-16a)

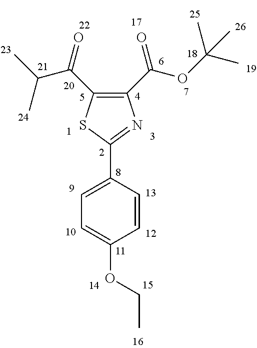 [SMILES: CCOc1ccc(-c2nc(C(=O)OC(C)(C)C)c(C(=O)C(C)C)s2)cc1]

According to the synthesis of RE-10, RE-13a (1.0 eq., 179 μmol, 68 mg) was oxidized using DMP. The crude product was purified via column chromatography (30 mL SiO₂, eluent CH/EA 6:1).

Yield: 43 mg (115 μmol, 64%) orange solid

C₂₀H₂₅NO₄S [375.48]

m.p.: 74° C.

R_{f}: 0.43 (CH/EA 4:1)

¹H-NMR (300 MHz, CDCl₃): δ 7.91 (d, J=8.7 Hz, 2H), 6.93 (d, J=8.7 Hz, 2H), 4.08 (q, J=6.9 Hz, 2H), 3.19 (dt, J=13.6, 6.8 Hz, 1H), 1.62 (s, 9H), 1.43 (t, J=6.9 Hz, 3H), 1.23 (d, J=6.8 Hz, 6H).

¹³C-NMR (75.5 MHz, CDCl₃): δ (ppm) 198.0, 169.9, 162.4, 161.8, 149.3, 136.2, 128.9, 125.1, 115.0, 83.6, 63.9, 40.9, 28.1, 18.9, 14.8.

### Example 174: tert-Butyl 5-(cyclopropanecarbonyl)-2-(4-ethoxyphenyl)thiazole-4-carboxylate (RE-16b)

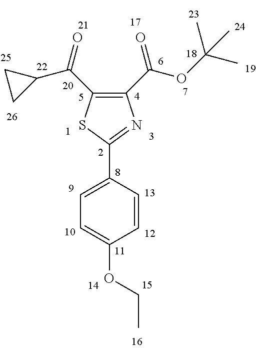 [SMILES: CCOc1ccc(-c2nc(C(=O)OC(C)(C)C)c(C(=O)C3CC3)s2)cc1]

According to the synthesis of RE-10, tert-butyl 5-(cyclopropyl(hydroxy)methyl)-2-(4-ethoxyphenyl)thiazole-4-carboxylate (1.0 eq., 202 μmol, 76 mg) was oxidized using DMP. The crude product was purified via column chromatography (40 mL SiO₂, eluent CH/EA 8:1).

Yield: 64 mg (171 μmol, 84%) orange solid

C₂₀H₂₃NO₄S [373.47]

m.p.: 119-122° C.

R_{f}: 0.35 (CH/EA 4:1)

¹H-NMR (300 MHz, CDCl₃): δ 7.85 (d, J=8.8 Hz, 2H), 6.86 (d, J=8.8 Hz, 2H), 4.02 (q, J=6.9 Hz, 2H), 2.42-2.26 (m, J=12.2, 7.8, 4.5 Hz, 1H), 1.57 (s, 9H), 1.37 (t, J=7.0 Hz, 3H), 1.28-1.18 (m, 2H), 1.06-0.95 (m, J=11.3, 3.5 Hz, 2H).

¹³C-NMR (75.5 MHz, CDCl₃): δ (ppm) 193.7, 170.1, 162.5, 161.8, 148.9, 137.7, 128.9, 125.2, 115.0, 83.6, 63.9, 28.1, 21.8, 14.8, 13.0.

### Example 175: tert-Butyl 5-allyl-2-(4-ethoxyphenyl)thiazole-4-carboxylate (AM-4-274)

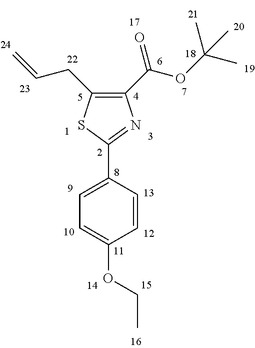 [SMILES: C=CCc1sc(-c2ccc(OCC)cc2)nc1C(=O)OC(C)(C)C]

According to the synthesis of RE-10, tert-butyl 5-bromo-2-(4-ethoxyphenyl)thiazole-4-carboxylate (1.0 eq, 312 μmol, 120 mg) was converted to the corresponding Grignard reagent and treated with allyl bromide (1.1 eq., 343 μmol, 30 μL). The crude product was purified via column chromatography (25 mL SiO₂, eluent CH/EA 9:1).

Yield: 90 mg (261 μmol, 83%) light yellow solid

C₁₉H₂₃NO₃S [345.46]

m.p.: 95° C.

R_{f}: 0.43 (CH/EA 6:1)

¹H-NMR (300 MHz, CDCl₃): δ (ppm) 7.90 (d, J=8.8 Hz, 2H), 6.94 (d, J=8.8 Hz, 2H), 6.05 (ddt, J=13.0, 10.0, 6.5 Hz, 1H), 5.32-5.03 (m, 2H), 4.10 (q, J=6.9 Hz, 2H), 3.96 (d, J=6.4 Hz, 2H), 1.66 (s, 9H), 1.46 (t, J=7.0 Hz, 3H).

¹³C-NMR (75.5 MHz, CDCl₃): δ (ppm) 164.4, 161.8, 160.8, 145.2, 143.2, 135.6, 128.3, 126.0, 117.3, 114.8, 82.1, 63.8, 32.2, 28.4, 14.9.

### Example 176: Ethyl 5-ethoxy-2-(4-ethoxyphenyl)thiazole-4-carboxylate (OKO-10)

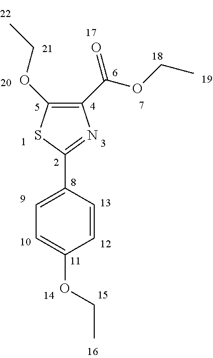 [SMILES: CCOC(=O)c1nc(-c2ccc(OCC)cc2)sc1OCC]

In an inert Schlenk flask equipped with magnetic stirring bar Na (3.3 eq., 1.87 mmol, 43 mg) was fully dissolved in 7 mL anhydrous EtOH. The solution was cooled down to 0° C. using an ice bath and ethyl 5-bromo-2-(4-ethoxyphenyl)thiazole-4-carboxylate (1.0 eq., 560 μmol, 200 mg) was added. The reaction was allowed to warm to RT. As conversion was not complete after 1 h, the reaction mixture was heated to 70° C. and stirred overnight. When full conversion could be observed via TLC, the reaction was quenched by the addition of ice and extracted with DCM (2×15 mL). The organic phase was washed with Brine (1×30 mL), dried over Na₂SO₄, filtered and the solvent was removed under reduced pressure. The crude product was purified via column chromatography (200 mL SiO₂, eluent CH/EA 4:1).

Yield: 45 mg (140 μmol, 25%) light yellow solid

C₁₆H₁₉NO₄S [321.39]

m.p.: 81° C.

R_{f}: 0.24 (CH/EA 4:1)

¹H-NMR (300 MHz, CDCl₃): δ (ppm) 7.78 (d, J=8.7 Hz, 2H), 6.91 (d, J=8.7 Hz, 2H), 4.41 (q, J=7.1 Hz, 2H), 4.30 (q, J=7.0 Hz, 2H), 4.07 (q, J=6.9 Hz, 2H), 1.55 (t, J=7.0 Hz, 3H), 1.47-1.36 (m, J=12.3, 6.1 Hz, 6H).

¹³C-NMR (75.5 MHz, CDCl₃): δ (ppm) 167.9, 161.9, 160.7, 153.1, 127.8, 127.4, 126.2, 114.8, 74.5. 63.78, 60.9, 15.0, 14.9, 14.6.

### Example 177: Isopropyl 2-(4-ethoxyphenyl)-5-(isopropoxythiazole-4-carboxylate (OKO-13)

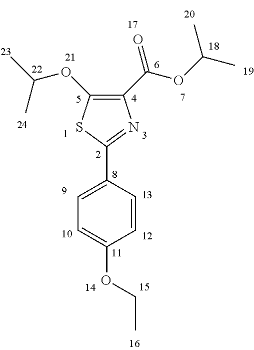 [SMILES: CCOc1ccc(-c2nc(C(=O)OC(C)C)c(OC(C)C)s2)cc1]

In an inert Schlenk flask equipped with magnetic stirring bar Na (3.6 eq., 2.04 mmol, 47 mg) was fully dissolved in 10 mL anhydrous isopropanol. Ethyl 5-chloro-2-(4-ethoxyphenyl)thiazole-4-carboxylate (1.0 eq., 560 μmol, 175 mg) was added and the reaction mixture was heated to 80° C. and stirred overnight. When full conversion could be observed via TLC, the reaction was quenched by the addition of ice and extracted with DCM (2×15 mL). The organic phase was washed with Brine (1×30 mL), dried over Na₂SO₄, filtered and the solvent was removed under reduced pressure. The crude product was purified via column chromatography (50 mL SiO₂, eluent CH/EA 8:1).

Yield: 52 mg (149 μmol, 27%) colorless solid

C₁₈H₂₃NO₄S [349.45]

R_{f}: 0.28 (CH/EA 4:1)

¹H-NMR (300 MHz, CDCl₃): δ (ppm) 7.78 (d, J=8.8 Hz, 2H), 6.90 (d, J=8.8 Hz, 2H), 5.39-5.10 (m, J=12.5, 6.2 Hz, 1H), 4.49-4.29 (m, J=12.1, 6.1 Hz, 1H), 4.06 (q, J=6.9 Hz, 2H), 1.56-1.30 (m, 15H).

¹³C-NMR (75.5 MHz, CDCl₃): δ (ppm) 166.1, 161.6, 160.0, 154.2, 129.8, 127.7, 126.3, 114.3, 82.7, 68.3, 63.7, 22.2. 22.1, 14.8.

### Intermediate Ethyl 2-chloro-5-iodothiazole-4-carboxylate (OKO-04)

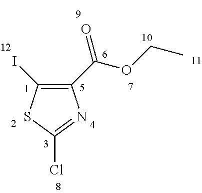

In an inert Schlenk flask equipped with magnetic stirring bar ethyl 2-amino-5-iodothiazole-4-carboxylate (1.0 eq., 1.69 mmol, 505 mg) and CuCl₂ (1.2 eq., 2.03 mmol, 271 mg) were dissolved in 8 mL MeCN anhydrous. The reaction mixture was heated to 80° C. and ^{t}BuONO (1.2 eq. 2.03 mmol, 270 μL) were added dropwise over 5 min. The reaction was stirred for another 15 min. When full conversion could be observed via TLC, the solvent was removed under reduced pressure. The residue was taken up in DCM (50 ml) and washed with water (5×200 H2O). The organic phase was dried over Na₂SO₄, filtered and the solvent was removed under reduced pressure. The crude product was purified via column chromatography (100 mL SiO₂, eluent CH/EA 12:1).

Yield: 132 mg (416 μmol, 25%) light yellow solid

C₆H₅ClINO₂S [317.53]

m.p.: 81° C.

R_{f}: 0.88 (DCM/MeOH 30:1)

¹H-NMR (300 MHz, CDCl₃): δ (ppm) 4.44 (q, J=7.1 Hz, 2H), 1.43 (t, J=7.1 Hz, 3H).

¹³C-NMR (75.5 MHz, CDCl₃): δ (ppm) 160.5, 156.8, 146.6, 80.1, 62.3, 14.4.

### Intermediate Ethyl 2-chloro-5-(trifluoromethyl)thiazole-4-carboxylate (OKO-07)

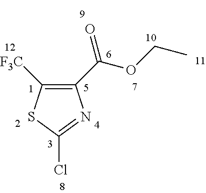

In a 25 mL round bottom flask equipped with magnetic stirring bar and Schienk adapter OKO-04 (1.0 eq., 334 μmol, 506 mg) and CuI (2.0 eq., 672 μmol, 128 mg) were dissolved in 2 mL DMF anhydrous. Difluorosulfonyl acetate (1.4 eq., 470 μmol, 140 μL) were added and the reaction mixture was stirred at 75° C. When full conversion could be observed via TLC, the reaction was quenched via the addition of water and extracted with EA (3×10 mL). The organic phase was dried over Na₂SO₄, filtered and the solvent was removed under reduced pressure. The crude product was purified via column chromatography (100 mL SiO₂, eluent CH/EA 20:1).

Yield: 46 mg (177 μmol, 53%) off-white solid

C₇H₅ClF₃NO₂S [259.63]

R_{f}: 0.63 (CH/EA 4:1)

¹H-NMR (300 MHz, CDCl₃): δ (ppm) 4.44 (q, J=7.1 Hz, 2H), 1.40 (t, J=7.1 Hz, 3H).

¹³C-NMR (75.5 MHz, CDCl₃): δ (ppm) 158.9, 153.6, 144.6, 144.6, 133.7, 133.2, 122.0, 118.4, 62.9, 14.0.

### Example 178: Ethyl 2-(4-ethoxyphenyl)-5-(trifluoromethyl)thiazole-4-carboxylate (OKO-08)

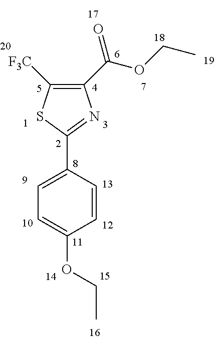 [SMILES: CCOC(=O)c1nc(-c2ccc(OCC)cc2)sc1C(F)(F)F]

In an inert Schienk flask equipped with magnetic stirring bar OKO-07 (1.0 eq., 177 μmol, 46 mg), 4-ethoxyphenylboronic acid (1.26 eq., 222 μmol, 37 mg), K₃PO₄ (2.0 eq., 354 μmol, 75 mg) and Pd[PPh₃]₄ (0.06 eq., 10 μmol, 12 mg) were dissolved in 10 mL anhydrous dioxane and stirred at 100° C. When full conversion could be observed via TLC after 2 h, the reaction mixture was cooled down to RT and filtered over a pad of Celite. The solvent was removed under reduced pressure and the crude product was purified via column chromatography (20 mL SiO₂, eluent CH/EA 10:1).

Yield: 32 mg (93 μmol, 52%) off-white solid

C₁₅H₁₄F₃NO₃S [345.34]

R_{f}: 0.48 (CH/EA 4:1)

¹H-NMR (300 MHz, CDCl₃): δ (ppm) 7.91 (d, J=8.8 Hz, 2H), 6.95 (d, J=8.8 Hz, 2H), 4.47 (q, J=7.1 Hz, 2H), 4.09 (q, J=6.9 Hz, 2H), 1.50-1.32 (m, J=12.3, 7.0 Hz, 6H).

¹³C-NMR (75.5 MHz, CDCl₃): δ (ppm) 169.0, 162.1, 160.5, 146.5, 130.0, 129.4, 128.9, 124.4, 123.2, 119.6, 115.1, 64.0, 62.4, 14.8, 14.1.

### Example 179: Ethyl (E)-2-styrylthiazole-4-carboxylate (AM-4-252)

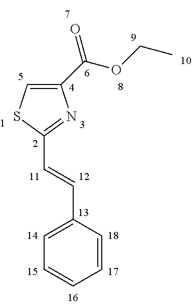 [SMILES: CCOC(=O)c1csc(/C=C/c2ccccc2)n1]

According to General Procedure A, ethyl 2-bromothiazole-4-carboxylate (1.0 eq. 847 μmol, 200 mg) was coupled with (E)-styrylboronic acid (1.2 eq. 1.02 mmol, 150 mg). The crude product was purified via column chromatography (50 ml SiO₂, eluent CH/EA 4:1) and preparative HPLC (method E).

Yield: 52 mg (200 μmol, 24%) colorless solid

C₁₄H₁₃NO₂S [259.32]

m.p.: 82° C.

R_{f}: 0.33 (CH/EA 4:1)

¹H-NMR (300 MHz, CDCl₃): δ (ppm) δ 8.08 (s, 1H), 7.60-7.29 (m, 7H), 4.45 (q, J=7.1 Hz, 2H), 1.42 (t, J=7.1 Hz, 3H).

¹³C-NMR (75.5 MHz, CDCl₃): δ (ppm) 167.8, 161.5, 147.9, 136.4, 135.4, 129.5, 129.1, 127.4, 126.5, 121.3, 61.7, 14.5.

### Example 180: tert-Butyl 2-(phenylethynyl)thiazole-4-carboxylate (AM-4-251)

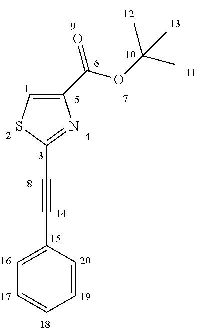 [SMILES: CC(C)(C)OC(=O)c1csc(C#Cc2ccccc2)n1]

In an inert Schienk flask equipped with magnetic stirring bar ethyl 2-bromo-4-thiazolecarboxylate (1.0 eq., 2.12 mmol, 500 mg), CuI (0.03 eq., 64 μmol, 12 mg), and PdCl₂(PPh3)₂ (0.03 eq., 64 μmol, 45 mg) were dissolved in 5 mL anhydrous DCM. Et₃N (7.0 eq., 14.8 mmol, 2.1 mL) and phenylacetylene (1.5 eq., 3.18 mmol, 350 μL) were added successively. The reaction mixture was heated to reflux and stirred overnight. When full conversion could be observed via GC-MS, the reaction mixture was cooled down to RT, diluted with 10 mL DCM and filtered over a pad of Celite. The solvent was removed under reduced pressure. It was tried to purify the crude product via column chromatography for three times, however major impurities could not be separated. Still the product was used for the next step.

Yield: 323 mg (1.13 mmol, 53%, with major impurities) light yellow solid

C₁₆H₁₅NO₂S [285.36]

m.p.: 136° C.

R_{f}: 0.49 (CH/EA 4:1)

¹H-NMR (300 MHz, CDCl₃): δ (ppm) 8.19 (s, 1H), 7.69-7.50 (m, 2H), 7.40 (dd, J=8.7, 4.4 Hz, 3H), 4.45 (q, J=7.1 Hz, 2H), 1.42 (t, J=7.1 Hz, 3H).

¹³C-NMR (75.5 MHz, CDCl₃): δ (ppm) 161.1, 149.5, 147.9, 132.2, 130.0, 128.7, 128.6, 121.1, 95.1, 81.91, 61.9, 14.5.

### Example 181: Ethyl 2-phenethylthiazole-4-carboxylate (AM-4-257)

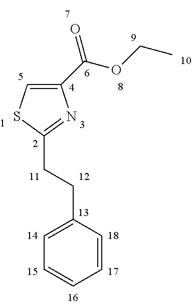 [SMILES: CCOC(=O)c1csc(CCc2ccccc2)n1]

An inert 50 mL round bottom flask equipped with magnetic stirring bar and Schlenk adapter was charged with AM-4-251 (50% pure, 1.0 eq., 538 0 μmol, 277 mg). The starting material was dissolved in 15 mL anhydrous EtOH and the solution was degassed. Pd/C 10% (0.1 eq., 54 μmol, 57 mg) was added and the reaction mixture was purged with H₂ and stirred vigorously overnight. When full conversion was observed via GC-MS, the reaction mixture was filtered over a pad of Celite and the solvent was removed under reduced pressure. The crude product was purified via column chromatography (50 mL SiO₂, eluent toluene/EA 15:1).

Yield: 123 mg (470 μmol, 87%) colorless oil

C₁₄H₁₅NO₂S [261.34]

R_{f}: 0.23 (toluene/EA 15:1)

¹H-NMR (300 MHz, CDCl₃): δ (ppm) 8.03 (s, 1H), 7.27 (ddd, J=19.9, 19.2, 13.6 Hz, 7H), 4.43 (q, J=7.1 Hz, 2H), 3.44-3.31 (m, 2H), 3.17-3.03 (m, 2H), 1.41 (t, J=7.1 Hz, 3H).

¹³C-NMR (75.5 MHz, CDCl₃): δ (ppm) 171.0, 161.6, 147.0, 140.0, 129.1, 128.7, 128.6, 127.1, 126.6, 61.6, 36.1, 35.4, 14.5.

### Examples 182 to 187

General Procedures

Suzuki Coupling Procedure A

A predried 20 mL Schlenk tube with magnetic stirring bar was charged with 5 mol-% PdCl₂(dppf), 1.0 eq aryl halide, 1.0-1.3 eq boronic acid and 2.1 eq cesium fluoride in anhydrous DME. The reaction mixture was degassed by evacuation and purging with Ar (repeated three times) and placed in an oil bath at 80° C. After stirring for 21-96 h (reaction control via TLC and GC/MS) the reaction mixture was cooled to RT and filtered through a pad of Celite®. The filter cake was washed with an appropriate amount of EtOAc, the volatiles were removed under reduced pressure and the crude residue was died in oil-pump vacuum. Pure product was obtained via flash column chromatography (SiO₂, cyclohexane/EtOAc or toluene/EtOAc), unless otherwise stated.

Suzuki Coupling Procedure B

A 15-50 mL Schlenk-tube with magnetic stirring bar was evacuated and purged with Ar (repeated three times). The Schlenk-tube was subsequently charged with 5 mol-% Pd(OAc)₂, 10 mol-% SPhos, 1.2 eq boronic acid, 1.0 eq aryl halide (if solid) and 1,4-dioxane. At this point 1.0 eq aryl halide (if liquid) and a 3.4 M K₃PO₄ solution (degassed) was added via Eppendorf® pipette in an Ar counterstream. The reaction mixture was degassed by evacuation and purging with Ar (repeated three times) and placed in an oil bath at 60° C. After stirring for 8-18 h (reaction control via TLC and GC/MS) the reaction mixture was cooled to RT, the phases separated and the organic phase filtered through a pad of Celite®. The filter cake was washed with an appropriate amount of EtOAc, the volatiles were removed under reduced pressure and the crude residue was died in oil-pump vacuum. Pure product was obtained via flash column chromatography (SiO₂, cyclohexane/EtOAc).

(Additional) Preparative HPLC Methods

Method A: 0-2 min 50% of H₂O and 50% CH₃CN, 2-17 min linear to 100% CH₃CN, 17-22 min 100% CH₃CN with a flow of 15 mLmin⁻¹.

Method B: 0-3 min 60% of H₂O and 40% CH₃CN, 3-37 min linear to 95% CH₃CN, 37-42 min 95% CH₃CN with a flow of 15 mLmin⁻¹.

### Example 182: Ethyl 2-phenylthiazole-4-carboxylate (CLF-3-197)

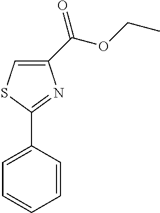 [SMILES: CCOC(=O)c1csc(-c2ccccc2)n1]

Phenylboronic acid (62.0 mg, 0.508 mmol, 1.2 eq), ethyl 2-bromothiazole-4-carboxylate (100.0 mg, 0.424 mmol, 1.0 eq) and potassium phosphate (726.5 mg, 3.423 mmol, 8.1 eq) in 6.0 mL 1,4-dioxane/H₂O 5:1 (v/v) were reacted in presence of Pd(OAc)₂ (4.8 mg, 0.021 mmol, 5 mol-%) and SPhos (17.4 mg, 0.042 mmol, 10 mol-%) for 18 h at 60° C. according to general procedure B. The crude product was purified via flash column chromatography (15 g SiO₂, cyclohexane/EtOAc 15:1 (v/v), column size 13×2 cm) to obtain the pure product as pale yellow wax-like compound.

Yield: 60.8 mg (0.261 mmol, 61%), pale yellow wax-like compound.

C₁₂H₁₁NO₂S [233.29 g/mol].

R_{f}=0.40 (cyclohexane/EtOAc=5:1 (v/v); staining: KMnO₄).

¹H NMR (300 MHz, CDCl₃): δ=8.15 (s, 1H, Ar—H), 8.06-7.94 (m, 2H, Ar—H), 7.54-7.36 (m, 3H, Ar—H), 4.45 (q, ³J=7.1 Hz, 2H, CH₂), 1.43 (t, ³J=7.1 Hz, 3H, CH₃).

¹³C NMR (76 MHz, CDCl₃): δ=169.0, 161.6, 148.2, 133.0, 130.8, 129.1, 127.1, 61.6, 14.5.

HRMS (DI-EI): Calcd. for C₁₂H₁₁NO₂S: 233.0511; found: 233.0511.

### Example 183: Ethyl 2-(4-(trifluoromethyl)phenyl)thiazole-4-carboxylate (CM-02)

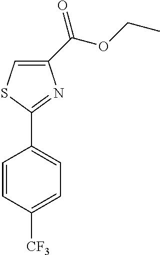 [SMILES: CCOC(=O)c1csc(-c2ccc(C(F)(F)F)cc2)n1]

4-(Trifluoromethyl)phenylboronic acid (95.4 mg, 0.502 mmol, 1.1 eq), ethyl 2-bromothiazole-4-carboxylate (107.9 mg, 0.457 mmol, 1.0 eq) and cesium fluoride (145.6 mg, 0.959 mmol, 2.1 eq) in 2.3 mL anhydrous DME were reacted in presence of PdCl₂(dppf) (16.7 mg, 0.023 mmol, 5 mol-%) for 21 h at 80° C. according to general procedure A. After 21 h the temperature was increased to 100° C. and the reaction mixture was stirred for another 22 h. The crude product was purified via flash column chromatography (15 g SiO₂, toluene/EtOAc 40:1 (v/v), column size 13×2 cm), followed by preparative RP-HPLC (method A).

Yield: 21.1 mg (0.070 mmol, 16%), colorless solid.

C₁₃H₁₀F₃NO₂S [301.28 g/mol].

mp=100° C.

R_{f}=0.27 (toluene/EtOAc=40:1 (v/v); staining: KMnO₄).

¹H NMR (300 MHz, CDCl₃): δ=8.22 (s, 1H, Ar—H), 8.13 (d, ³J=8.1 Hz, 2H, Ar—H), 7.71 (d, ³J=8.2 Hz, 2H, Ar—H), 4.46 (q, ³J=7.1 Hz, 2H, CH₂), 1.44 (t, ³J=7.1 Hz, 3H, CH₃).

¹³C NMR (76 MHz, CDCl₃): δ=167.1, 161.4, 148.7, 136.0, 132.5 (q, ²J_{CF}=32.8 Hz), 128.0, 127.4, 126.2 (q, ³J_{CF}=3.8 Hz), 123.9 (q, ¹J_{CF}=272.3 Hz), 61.8, 14.5.

HRMS (DI-EI): Calcd. for C₁₃H₁₀F₃NO₂S: 301.0384; found: 301.0389.

### Example 184: Ethyl 2-(p-tolyl)thiazole-4-carboxylate (DA-04)

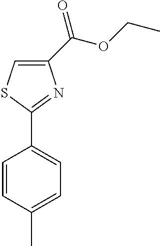 [SMILES: CCOC(=O)c1csc(-c2ccc(C)cc2)n1]

p-Tolylboronic acid (62.2 mg, 0.451 mmol, 1.1 eq), ethyl 2-bromothiazole-4-carboxylate (100.2 mg, 0.424 mmol, 1.0 eq) and cesium fluoride (136.8 mg, 0.900 mmol, 2.1 eq) in 2.0 mL anhydrous DME were reacted in presence of PdCl₂(dppf) (16.0 mg, 0.022 mmol, 5 mol-%) for 92 h at 80° C. according to general procedure A. The crude product was purified via flash column chromatography (20 g SiO₂, cyclohexane/EtOAc 10:1 (v/v), column size 13×2 cm), followed by preparative RP-HPLC (method B).

Yield: 58.1 mg (0.235 mmol, 55%), pale yellow solid.

C₁₃H₁₃NO₂S [247.31 g/mol].

mp=39-41° C.

R_{f}=0.16 (cyclohexane/EtOAc=10:1 (v/v); staining: KMnO₄).

¹H NMR (300 MHz, CDCl₃): δ=8.11 (s, 1H, Ar—H), 7.89 (d, ³J=8.0 Hz, 2H, Ar—H), 7.25 (d, 3J=8.0 Hz, 2H, Ar—H), 4.44 (q, ³J=7.1 Hz, 2H, CH₂), 2.39 (s, 3H, CH₃), 1.42 (t, ³J=7.1 Hz, 3H, CH₂CH₃).

¹³C NMR (76 MHz, CDCl₃): δ=169.2, 161.6, 148.1, 141.2, 130.3, 129.8, 127.0, 126.8, 61.6, 21.6, 14.5.

HRMS (DI-EI): Calcd. for C₁₃H₁₃NO₂S: 247.0667; found: 291.0655.

### Example 185: Ethyl 2-(4-isopropylphenyl)thiazole-4-carboxylate (DA-06)

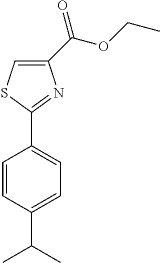 [SMILES: CCOC(=O)c1csc(-c2ccc(C(C)C)cc2)n1]

4-Isopropylphenylboronic acid (73.2 mg, 0.446 mmol, 1.05 eq), ethyl 2-bromothiazole-4-carboxylate (100.6 mg, 0.424 mmol, 1.0 eq) and cesium fluoride (137.7 mg, 0.906 mmol, 2.1 eq) in 2.0 mL anhydrous DME were reacted in presence of PdCl₂(dppf) (17.2 mg, 0.023 mmol, 5 mol-%) for 50 h at 80° C. according to general procedure A. The crude product was purified via flash column chromatography (17 g SiO₂, cyclohexane/EtOAc 15:1 (v/v), column size 12×2 cm).

Yield: 23.5 mg (0.085 mmol, 20%), brownish oil.

C₁₅H₁₇NO₂S [275.37 g/mol].

R_{f}=0.35 (cyclohexane/EtOAc=5:1 (v/v); staining: KMnO₄).

¹H NMR (300 MHz, CDCl₃): δ=8.11 (s, 1H, Ar—H), 7.93 (d, ³J=8.3 Hz, 2H, Ar—H), 7.30 (d, ³J=8.0 Hz, 2H, Ar—H), 4.45 (q, ³J=7.1 Hz, 2H, CH₂), 3.03-2.87 (m, 1H, CH(CH₃)₂), 1.43 (t, ³J=7.1 Hz, 3H, CH₂CH₃), 1.28 (d, ³J=6.9 Hz, 6H, CH(CH₃)₂).

¹³C NMR (76 MHz, CDCl₃): δ=169.2, 161.7, 152.1, 148.1, 130.7, 127.2, 126.8, 61.6, 34.2, 23.9, 14.5.

HRMS (DI-EI): Calcd. for C₁₅H₁₇NO₂S: 275.0980; found: 275.0978.

### Example 186: Ethyl 2-(4-(tert-butyl)phenyl)thiazole-4-carboxylate (DA-08)

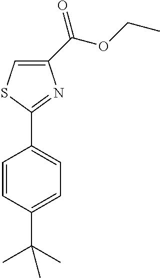 [SMILES: CCOC(=O)c1csc(-c2ccc(C(C)(C)C)cc2)n1]

4-tert-Butylphenylboronic acid (101.5 mg, 0.553 mmol, 1.3 eq), ethyl 2-bromothiazole-4-carboxylate (100.5 mg, 0.426 mmol, 1.0 eq) and cesium fluoride (139.1 mg, 0.915 mmol, 2.1 eq) in 2.0 mL anhydrous DME were reacted in presence of PdCl₂(dppf) (16.1 mg, 0.022 mmol, 5 mol-%) for 71 h at 80° C. according to general procedure A. The crude product was purified 5 via flash column chromatography (17 g SiO₂, cyclohexane/EtOAc 15:1 (v/v), column size 11×2 cm).

Yield: 60.5 mg (0.209 mmol, 49%), colorless solid.

C₁₆H₁₉NO₂S [289.39 g/mol].

mp=50-52° C.

R_{f}=0.44 (cyclohexane/EtOAc=5:1 (v/v); staining: KMnO₄).

¹H NMR (300 MHz, CDCl₃): δ=8.12 (s, 1H, Ar—H), 7.93 (d, ³J=8.3 Hz, 2H, Ar—H), 7.46 (d, ³J=8.3 Hz, 2H, Ar—H), 4.44 (q, ³J=7.1 Hz, 2H, CH₂), 1.42 (t, ³J=7.1 Hz, 3H, CH₃), 1.34 (s, 9H, (CH₃)₃).

¹³C NMR (76 MHz, CDCl₃): δ=169.0, 161.5, 154.2, 148.0, 130.2, 126.8, 126.7, 125.9, 61.4, 35.0, 31.2, 14.4.

HRMS (DI-EI): Calcd. for C₁₆H₁₉NO₂S: 289.1136; found: 289.1130.

### Example 187: Ethyl 2-(4-(benzyloxy)phenyl)thiazole-4-carboxylate (BB-3-106)

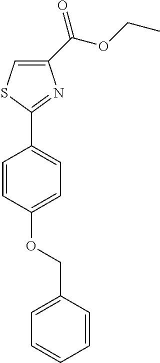 [SMILES: CCOC(=O)c1csc(-c2ccc(OCc3ccccc3)cc2)n1]

4-Benzyloxyphenylboronic acid (272.0 mg, 1.193 mmol, 1.2 eq), ethyl 2-bromothiazole-4-carboxylate (234.6 mg, 0.994 mmol, 1.0 eq) and potassium phosphate (1.59 g, 7.50 mmol, 7.5 eq) in 17 mL 1,4-dioxane/H₂O 5:1 (v/v) were reacted in presence of Pd(OAc)₂ (11.2 mg, 0.050 mmol, 5 mol-%) and SPhos (40.6 mg, 0.099 mmol, 10 mol-%) for 8 h at 60° C. according to general procedure B. The crude product was purified via flash column chromatography (46 g SiO₂, cyclohexane/EtOAc 6:1 (v/v), column size 8×3.5 cm) to obtain the pure product as pale pale brown solid.

Yield: 212.0 mg (0.625 mmol, 63%), pale brown solid.

C₁₉H₁₇NO₃S [339.41 g/mol].

mp=112-115° C.

R_{f}=0.30 (cyclohexane/EtOAc=4:1 (v/v); staining: KMnO₄).

¹H NMR (300 MHz, CDCl₃): δ=8.09 (s, 1H, Ar—H), 7.95 (d, ³J=8.6 Hz, 2H, Ar—H), 7.54-7.29 (m, 5H, Ar—H), 7.03 (d, ³J=8.6 Hz, 2H, Ar—H), 5.12 (s, 2H, CH₂), 4.44 (q, ³J=7.1 Hz, 2H, CH₂CH₃), 1.43 (t, ³J=7.1 Hz, 3H, CH₂CH₃).

¹³C NMR (76 MHz, CDCl₃): δ=13C NMR (76 MHz, CDCl₃) δ 168.8, 161.7, 160.9, 148.0, 136.5, 128.8, 128.7, 128.3, 127.6, 126.4, 126.1, 115.3, 70.3, 61.6, 14.5.

HRMS (DI-EI): Calcd. for C₁₂H₁₁NO₂S: 339.0929; found: 339.0916.

### Examples 188 to 206

General Procedures

General Procedure A (Suzuki Coupling)

A 50 mL Schlenk-tube with magnetic stirring bar was evacuated and purged with Ar (repeated three times). The Schlenk-tube was subsequently charged with 5 mol-% XPhos Pd G4, 1.5 eq boronic acid (or its derivative), 2.0 eq potassium carbonate, 1.0 eq aryl halide (if solid) and 1,4-dioxane/H₂O 5:1 (v/v). At this point 1.0 eq aryl halide (if liquid) was added via Eppendorf® pipette in an Ar counterstream. The reaction mixture was degassed by evacuation and purging with Ar (repeated three times) and placed in an oil bath at 80° C. After stirring for 22-120 h (reaction control via TLC and HPLC/MS) the reaction mixture was cooled to RT and the filtered through a pad of Celite®. The filter cake was washed with an appropriate amount of EtOAc and the volatiles were removed under reduced pressure. Pure product was obtained via flash column chromatography (SiO₂, cyclohexane/EtOAc), followed by RP preparative HPLC.

General Procedure B (Suzuki Coupling)

A 15 mL Schlenk tube with magnetic stirring bar was dried with a heatgun, evacuated and purged with Ar. The Schlenk tube was charged with 1.0 eq isopropyl 6-chloro-4-methoxypicolinate (MC-22) 1.2 eq of the corresponding boronic acid, 2.1 eq cesium fluoride and 5.0 mol-% PdCl₂(dppf) in DME abs. The red suspension was degassed (3×vacuum/Ar cycles) and heated to 80° C. in a pre-heated oil bath. After 5 d the mixture was cooled to RT, filtered through a pad of Celite® and the volatiles were removed under reduced pressure. The crude product was purified via flash column chromatography (SiO₂, cyclohexane/EtOAc), followed by RP preparative HPLC.

General Procedure C (DCC-Mediated Esterification)

A 15 mL Schlenk-tube with magnetic stirring bar was evacuated and purged with Ar (repeated three times). The Schlenk tube was charged with a mixture of 71.7 mg (0.295 mmol, 1.0 eq) 6-(4-ethoxyphenyl)picolinic acid (see NG-384) and 1.5-3.0 eq of the corresponding alcohol in 1.5 mL CH₂Cl₂ abs. The suspension was cooled to 0° C. and DMAP (3.6 mg, 29 μmol, 0.1 eq), followed by 84.8 mg (0.442 mmol, 1.5 eq) of EDC hydrochloride were added. The ice bath was removed and the pale-yellow solution was left to spontaneous warmup overnight (reaction control via TLC). The reaction mixture was diluted with 4.5 mL CH₂Cl₂, washed with 1 M HCl (2×3 mL), satd. NaHCO₃ (3 mL) and H₂O (3 mL). The phases were separated and the org. phase was dried over Na₂SO₄, filtered, and the solvent was removed under reduced pressure. The crude product was purified via flash column chromatography (SiO2, cyclohexane/EtOAc).

General Procedure D (DCC-Mediated Esterification)

A 15 mL Schlenk-tube with magnetic stirring bar was evacuated and purged with Ar (repeated three times). The Schienk tube was charged with a mixture of 71.7 mg (0.295 mmol, 1.0 eq) 6-(4-ethoxyphenyl)picolinic acid (see NG-384) and 3.0 eq of the corresponding alcohol in 1.5 mL CH₂Cl₂ abs. The suspension was cooled to 0° C. and DMAP (3.6 mg, 29 μmol, 0.1 eq), followed by 84.8 mg (0.442 mmol, 1.5 eq) of EDC hydrochloride were added. The ice bath was removed and the pale-yellow solution was left to spontaneous warmup overnight (reaction control via TLC). The reaction mixture was diluted with 4.5 mL CH₂Cl₂, and washed with H₂O (3×3 mL). The phases were separated and the org. phase was dried over Na₂SO₄, filtered, and the solvent was removed under reduced pressure. The crude product was purified via flash column chromatography (SiO2, cyclohexane/EtOAc).

General Procedure E (DCC-Mediated Esterification)

A 15 mL Schlenk tube with magnetic stirring bar was dried with a heatgun, evacuated and purged with Ar. The Schlenk tube was charged with 40.0 mg (0.139 mmol, 1.0 eq) 6-(4-isopropoxyphenyl)-4-methoxypicolinic acid (see CLF-5-397) and 0.7 mL CH₂Cl₂ abs. The suspension was cooled to 0° C. (ice bath) and 3.0 eq of the corresponding alcohol, 1.7 mg (14 μmol, 0.1 eq) DMAP and 40.0 mg (0.209 mmol, 1.5 eq) EDC.HCl were added. The reaction mixture was stirred and left to spontaneous warm up to RT. After 3-16 h the yellow suspension was diluted with 2.2 mL CH₂Cl₂, washed with H₂O (3×1.5 mL) and the phases separated. The organic phase was dried over Na₂SO₄, filtered, and the solvent was removed under reduced pressure. The crude product was purified via flash column chromatography (SiO₂, cyclohexane/EtOAc).

(Additional) Preparative HPLC Methods

Method A: 26° C., flow rate 15 mL/min; 0.0-2.0 min MeCN/H₂O=50:50 (v/v), 2.0-17.0 min linear increase to MeCN/H₂O=100:0 (v/v), 17.0-22.0 min hold MeCN/H₂O=100:0 (v/v), 22.0-23.0 min return to initial conditions.

Method B: 26° C., flow rate 15 mL/min; 0.0-2.0 min MeCN/H₂O=10:90 (v/v), 2.0-17.0 min linear increase to MeCN/H₂O=100:0 (v/v), 17.0-27.0 min hold MeCN/H₂O=100:0 (v/v), 27.0-28.0 min return to initial conditions.

Method C: 26° C., flow rate 15 mL/min; 0.0-5.0 min MeCN/H₂O=30:70 (v/v), 5.0-20.0 min linear increase to MeCN/H₂O=100:0 (v/v), 20.0-30.0 min hold MeCN/H₂O=100:0 (v/v), 30.0-31.0 min return to initial conditions.

### Example 188: Ethyl 2-(4-hydroxyphenyl)thiazole-4-carboxylate

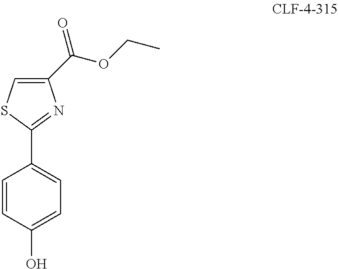 [SMILES: CCOC(=O)c1csc(-c2ccc(O)cc2)n1]

4-Hydroxyphenylboronic acid (87.6 mg, 0.635 mmol, 1.5 eq), ethyl 2-bromothiazole-4-carboxylate (100 mg, 0.424 mmol, 1.0 eq) and potassium carbonate (117 mg, 0.847 mmol, 2.0 eq) in 6.0 mL 1,4-dioxane/H₂O 5:1 (v/v) were reacted in presence of XPhos Pd G4 (18.2 mg, 21.2 μmol, 5 mol-%) for 22 h at 80° C. according to general procedure A. The crude product was purified via flash column chromatography (16 g SiO₂, cyclohexane/EtOAc 5:1 (v/v), column size 11×2 cm), followed by preparative RP-HPLC (method A).

Yield: 35.2 mg (0.141 mmol, 33%), colorless solid.

mp=171° C.

C₁₂H₁₁NO₃S [249.28 g/mol].

R_{f}=0.22 (cyclohexane/EtOAc=5:1 (v/v); staining: CAM).

¹H NMR (300 MHz, MeOD-d₄): δ=8.25 (s, 1H, Ar—H), 7.85 (d, ³J=8.7 Hz 1H, Ar—H), 6.88 (d, ³J=8.7 Hz, 1H, Ar—H), 4.40 (q, ³J=7.1 Hz, 2H, CH₂), 1.40 (t, ³J=7.1 Hz, 3H, CH₃). ¹³C NMR (76 MHz, MeOD-d₄): δ=171.1, 162.9, 161.6, 148.4, 129.6 (2C), 127.8, 125.6, 116.9 (2C), 62.5, 14.6.

HRMS (DI-EI): Calcd. m/z for C₁₂H₁₁NO₃S: 249.0460; found: 249.0461.

### Example 189: Thiophen-2-ylmethyl 6-(4-ethoxyphenyl)picolinate

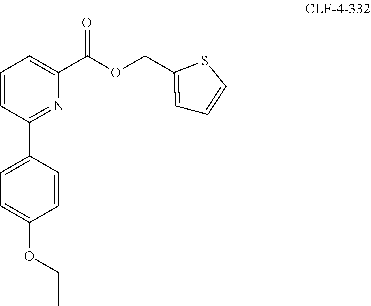 [SMILES: CCOc1ccc(-c2cccc(C(=O)OCc3cccs3)n2)cc1]

According to general procedure C; 101 mg (0.884 mmol, 3.0 eq) 2-thiophenemethanol, 18 h reaction time. Purification was performed via flash column chromatography (13 g SiO₂, cyclohexane/EtOAc 5:1 (v/v), column size 10×2 cm).

Yield: 67.9 mg (0.200 mmol, 68%), colorless solid.

C₁₉H₁₇NO₃S [339.41 g/mol].

mp=93-94° C.

R_{f}=0.38 (cyclohexane/EtOAc=5:1 (v/v); staining: CAM).

¹H NMR (300 MHz, CDCl₃): δ=8.14-7.91 (m, 3H, Ar—H), 7.89-7.74 (m, 2H, Ar—H), 7.38-7.30 (m, 1H, Ar—H), 7.23 (d, ³J=2.6 Hz, 1H, Ar—H), 7.09-6.91 (m, 3H, Ar—H), 5.61 (s, 2H, CH₂), 4.09 (q, ³J=6.9 Hz, 2H, CH₂), 1.44 (t, ³J=6.9 Hz, 3H, CH₃).

¹³C NMR (76 MHz, CDCl₃): δ=165.3, 160.4, 157.5, 147.8, 137.8, 137.6, 130.9, 128.7, 128.6 (2C), 127.1, 126.9, 122.9, 122.9′, 114.8 (2C), 63.7, 61.7, 14.9.

HRMS (DI-EI): Calcd. m/z for C₁₉H₁₇NO₃S: 339.0929; found: 339.0929.

### Example 190: Furan-2-ylmethyl 6-(4-ethoxyphenyl)picolinate

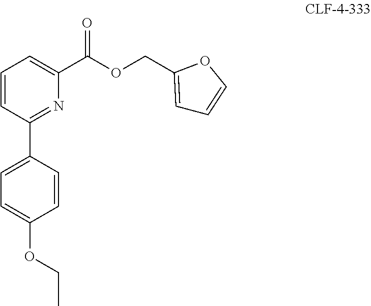 [SMILES: CCOc1ccc(-c2cccc(C(=O)OCc3ccco3)n2)cc1]

According to general procedure C; 86.7 mg (0.884 mmol, 3.0 eq) furfuryl alcohol, 18 h reaction time. Purification was performed via flash column chromatography (14 g SiO₂, cyclohexane/EtOAc 5:1 (v/v), column size 7×2.5 cm).

Yield: 64.5 mg (0.199 mmol, 68%), off-white solid.

C₁₉H₁₇NO₄ [323.35 g/mol].

mp=76-80° C.

R_{f}=0.30 (cyclohexane/EtOAc=5:1 (v/v); staining: CAM).

¹H NMR (300 MHz, CDCl₃): δ=8.10-7.90 (m, 3H, Ar—H), 7.89-7.74 (m, 2H, Ar—H), 7.55-7.37 (m, 1H, Ar—H), 6.98 (d, ³J=8.7 Hz, 2H, Ar—H), 6.54 (d, ³J=2.8 Hz, 2H, Ar—H), 6.45-6.32 (m, 1H, Ar—H), 5.40 (s, 2H, CH₂) 4.09 (q, ³J=6.9 Hz, 2H, CH₂), 1.44 (t, ³J=7.0 Hz, 3H, CH₃). ¹³C NMR (76 MHz, CDCl₃): δ=165.2, 160.4, 157.5, 149.48, 147.8, 143.4, 137.6, 131.0, 128.6 (2C), 122.9 (2C), 114.8 (2C), 111.3, 110.8, 63.7, 59.2, 14.9.

HRMS (DI-EI): Calcd. m/z for C₁₉H₁₇NO₄: 323.1158; found: 323.1155.

### Example 191: 2-(Furan-2-yl)ethyl 6-(4-ethoxyphenyl)picolinate

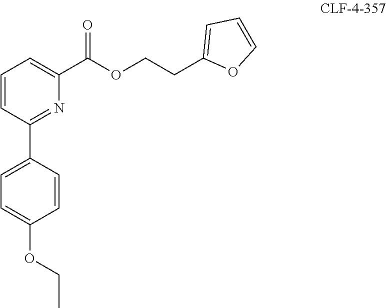 [SMILES: CCOc1ccc(-c2cccc(C(=O)OCCc3ccco3)n2)cc1]

According to general procedure C; 49.6 mg (0.442 mmol, 1.5 eq) 2-furanethanol, 20 h reaction time. Purification was performed via flash column chromatography (9 g SiO₂, cyclohexane/EtOAc 5:1 (v/v), column size 7×2 cm).

Yield: 60.8 mg (0.180 mmol, 61%), colorless solid.

C₂₀H₁₉NO₄ [337.38 g/mol].

mp=82-84° C.

R_{f}=0.31 (cyclohexane/EtOAc=5:1 (v/v); staining: CAM).

¹H NMR (300 MHz, CDCl₃): δ=8.10-7.75 (m, 5H, Ar—H), 7.42-7.30 (m, 1H, Ar—H), 6.99 (d, ³J=8.8 Hz, 2H, Ar—H), 6.39-6.26 (m, 1H, Ar—H), 6.25-6.13 (m, 1H, Ar—H), 4.65 (t, ³J=6.9 Hz, 2H, CH₂CH₂), 4.09 (q, ³J=6.9 Hz, 2H, CH₂CH₃), 3.19 (t, ³J=6.8 Hz, 2H, CH₂CH₂), 1.44 (t, ³J=7.0 Hz, 3H, CH₂CH₃).

¹³C NMR (76 MHz, CDCl₃): δ=165.4, 160.4, 157.4, 151.9, 148.0, 141.6, 137.6, 131.0, 128.6 (2C), 122.8, 122.7, 114.8 (2C), 110.4, 106.7, 63.7, 63.6, 27.9, 14.9.

HRMS (DI-EI): Calcd. m/z for C₂₀H₁₉NO₄: 337.1314; found: 337.1330.

### Example 192: Pyridin-2-ylmethyl 6-(4-ethoxyphenyl)picolinate

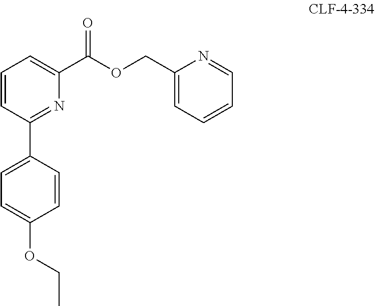 [SMILES: CCOc1ccc(-c2cccc(C(=O)OCc3ccccn3)n2)cc1]

According to general procedure C; 96.5 mg (0.884 mmol, 3.0 eq) 2-pyridinemethanol, 19 h reaction time. Purification was performed via flash column chromatography (13 g SiO₂, cyclohexane/EtOAc 5:1 (v/v), column size 7×2 cm).

Yield: 56.7 mg (0.170 mmol, 57%), colorless solid.

C₂₀H₁₈N₂O₃ [334.38 g/mol].

mp=124-127° C.

R_{f}=0.37 (cyclohexane/EtOAc=1:1 (v/v); staining: CAM).

¹H NMR (300 MHz, CDCl₃): δ=8.62 (d, J=4.0 Hz, 1H, Ar—H), 8.15-7.94 (m, 3H, Ar—H), 7.92-7.78 (m, 2H, Ar—H), 7.77-7.66 (m, 1H, Ar—H), 7.55 (d, ³J=7.7 Hz, 1H, Ar—H), 7.32-7.16 (m, 1H, Ar—H), 6.99 (d, ³J=8.8 Hz, 2H, Ar—H), 5.58 (s, 2H, CH₂), 4.09 (q, ³J=6.9 Hz, 2H, CH₂), 1.44 (t, ³J=7.0 Hz, 3H, CH₃).

¹³C NMR (76 MHz, CDCl₃): δ=165.3, 160.4, 157.5, 156.01, 149.5, 147.8, 137.6, 137.0, 130.9, 128.6 (2C), 123.0 (3C), 121.9, 114.8 (2C), 67.8, 63.7, 14.9.

HRMS (DI-EI): Calcd. m/z for C₂₀H₁₈N₂O₃: 334.1317; found: 334.1313.

### Example 193: Pyridin-3-ylmethyl 6-(4-ethoxyphenyl)picolinate

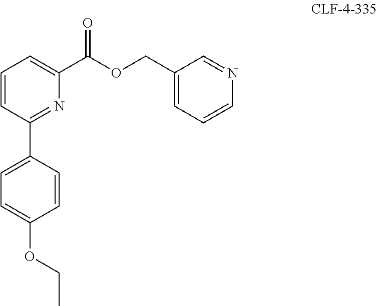 [SMILES: CCOc1ccc(-c2cccc(C(=O)OCc3cccnc3)n2)cc1]

According to general procedure D; 96.5 mg (0.884 mmol, 3.0 eq) 3-pyridinemethanol, 17 h reaction time. Purification was performed via flash column chromatography (10 g SiO₂, cyclohexane/EtOAc 1:1 (v/v), column size 8×2 cm).

Yield: 71.3 mg (0.213 mmol, 72%), colorless solid.

C₂₀H₁₈N₂O₃ [334.38 g/mol].

mp=107-110° C.

R_{f}=0.23 (cyclohexane/EtOAc=1:1 (v/v); staining: CAM).

¹H NMR (300 MHz, CDCl₃): δ=8.78 (s, 1H, Ar—H), 8.68-8.52 (m, 1H, Ar—H), 8.15-7.92 (m, 3H, Ar—H), 7.91-7.72 (m, 3H, Ar—H), 7.42-7.28 (m, 1H, Ar—H), 6.98 (d, ³J=8.8 Hz, 2H, Ar—H), 5.47 (s, 2H, CH₂), 4.08 (q, ³J=6.9 Hz, 2H, CH₂), 1.43 (t, ³J=7.0 Hz, 3H, CH₃).

¹³C NMR (76 MHz, CDCl₃): δ=165.4, 160.4, 157.6, 149.9, 149.8, 147.6, 137.7, 136.3, 131.7, 130.8, 128.6 (2C), 123.6, 123.1, 122.8, 114.9 (2C), 64.8, 63.7, 14.9.

HRMS (DI-EI): Calcd. m/z for C₂₀H₁₈N₂O₃: 334.1317; found: 334.1312.

### Example 194: Pyridin-4-ylmethyl 6-(4-ethoxyphenyl)picolinate

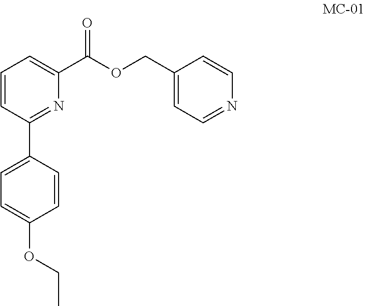 [SMILES: CCOc1ccc(-c2cccc(C(=O)OCc3ccncc3)n2)cc1]

According to general procedure D; 96.5 mg (0.884 mmol, 3.0 eq) 4-pyridinemethanol, 17 h reaction time. Purification was performed via flash column chromatography (10 g SiO₂, cyclohexane/EtOAc 1:1 (v/v), column size 7×2 cm).

Yield: 61.8 mg (0.185 mmol, 63%), pale yellow solid.

C₂₀H₁₈N₂O₃[334.38 g/mol].

mp=101-106° C.

R_{f}=0.78 (CH₂Cl₂/MeOH=15:1 (v/v), staining: CAM).

¹H NMR (300 MHz, CDCl₃): δ=8.63 (d, ³J=4.7 Hz, 2H, NCH), 8.03-7.98 (m, 3H, 2×Ph-H and 1×Py-H), 7.93-7.78 (m, 2H, Py-H), 7.40 (d, ³J=4.8 Hz, 2H, NCHCH), 6.99 (d, ³J=8.8 Hz, 2H, Ph-H), 5.46 (s, 2H, CO₂CH₂), 4.09 (q, ³J=7.0 Hz, 2H, CH₂CH₃), 1.44 (t, ³J=7.0 Hz, 3H, CH₃).

¹³C NMR (76 MHz, CDCl₃): δ=165.2, 160.5, 157.6, 150.2 (2C), 147.5, 145.1, 137.7, 130.8, 128.6 (2C), 123.2, 122.9, 122.0 (2C), 114.9 (2C), 65.3, 63.7, 14.9.

HRMS (DI-EI): Calcd. m/z for C₂₀H₁₈N₂O₃: 334.1317; found: 334.1319.

### Example 195: 2-(Thiophen-2-yl)ethyl 6-(4-ethoxyphenyl)picolinate

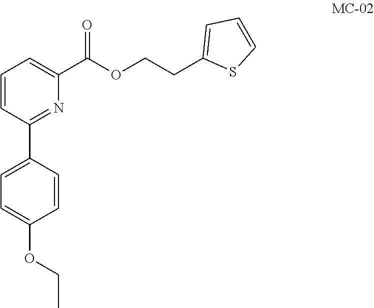 [SMILES: CCOc1ccc(-c2cccc(C(=O)OCCc3cccs3)n2)cc1]

According to general procedure D; 271 μL (0.884 mmol, 3.0 eq) 2-thiopheneethanol, 15 h reaction time. Purification was performed via flash column chromatography (30 g SiO₂, cyclohexane/EtOAc 10:1 (v/v), column size 10×3 cm).

Yield: 53.4 mg (0.151 mmol, 51%), beige solid.

C₂₀H₁₉NO₃S [353.44 g/mol].

mp=73-78° C.

R_{f}=0.94 (CH₂Cl₂/MeOH=15:1, staining: CAM).

¹H NMR (300 MHz, CDCl₃): δ=8.05 (d, ³J=8.8 Hz, 2H, Ph-H), 8.00-7.95 (m, 1H, Py-H), 7.88-7.81 (m, 2H, Py-H), 7.23-7.13 (m, 1H, SCH), 7.08-6.91 (m, 4H, 2×Ph-H and 2×thiophene), 4.63 (t, ³J=6.8, 2H, OCH₂CH₂), 4.10 (q, 3J=6.9 Hz, 2H, CH₂CH₃), 3.37 (t, ³J=6.7 Hz, 2H, OCH₂CH₂) 1.45 (t, ³J=7.0, 3H, CH₃).

¹³C NMR (76 MHz, CDCl₃): δ=165.5, 160.4, 157.4, 147.9, 139.9, 137.6, 131.0, 128.6 (2C), 127.1, 125.9, 124.2, 122.8 (2C), 114.8 (2C), 65.9, 63.7, 29.5, 14.9.

HRMS (DI-EI): Calcd. m/z for C₂₀H₁₉NO₃S: 353.1086; found: 353.1093.

### Example 196: 2-(Thiophen-3-yl)ethyl 6-(4-ethoxyphenyl)picolinate

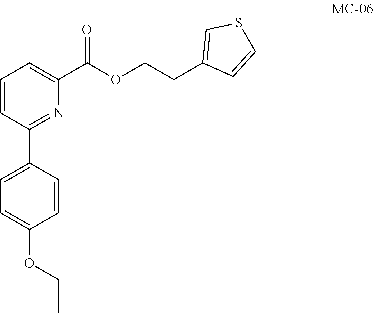 [SMILES: CCOc1ccc(-c2cccc(C(=O)OCCc3ccsc3)n2)cc1]

According to general procedure D; 99.1 μL (0.884 mmol, 3.0 eq) 3-thiopheneethanol, 16 h reaction time. Purification was performed via flash column chromatography (20 g SiO₂, cyclohexane/EtOAc 10:1 (v/v), column size 10×3 cm).

Yield: 61.5 mg (0.174 mmol, 59%), colorless solid.

C₂₀H₁₉NO₃S [353.44 g/mol].

mp=73-76° C.

R_{f}=0.96 (CH₂Cl₂/MeOH=15:1 (v/v), staining: CAM).

¹H NMR (300 MHz, CDCl₃): δ=8.04 (d, ³J=8.8 Hz, 2H, Ph-H), 7.98-7.89 (m, 1H, Py-H), 7.88-7.80 (m, 2H, Py-H), 7.33-7.24 (m, 1H, SCHCH), 7.21-7.09 (m, 2H, Ar—H from thiophene), 7.00 (d, ³J=8.8 Hz, 2H, Ph-H), 4.61 (t, ³J=6.9 Hz, 2H, OCH₂CH₂), 4.11 (q, ³J=6.9 Hz, 2H, CH₂CH₃), 3.18 (t, ³J=6.8 Hz, 2H, OCH₂CH₂), 1.45 (t, ³J=7.0 Hz, 3H, CH₃).

¹³C NMR (76 MHz, CDCl₃): δ=165.6, 160.4, 157.4, 148.1, 138.2, 137.6, 131.0, 128.7, 128.6 (2C), 125.7, 122.8, 122.6, 122.0, 114.9 (2C), 65.6, 63.7, 29.8, 14.92.

HRMS (DI-EI): Calcd. m/z for C₂₀H₁₉NO₃S: 353.1085; found: 353.1089.

### Example 197: Thiophen-3-ylmethyl 6-(4-ethoxyphenyl)picolinate

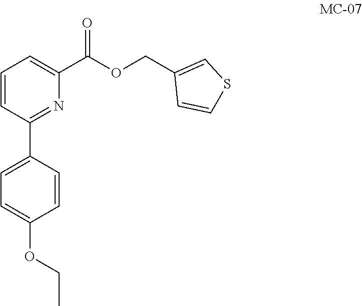 [SMILES: CCOc1ccc(-c2cccc(C(=O)OCc3ccsc3)n2)cc1]

According to general procedure D; 83.4 μL (0.884 mmol, 3.0 eq) 3-thiophenemethanol, 19 h reaction time. Purification was performed via flash column chromatography (15 g SiO₂, cyclohexane/EtOAc 10:1 (v/v), column size 10×2 cm).

Yield: 61.1 mg (0.181 mmol, 62%), beige solid.

C₁₉H₁₇NO₃S [339.41 g/mol].

mp=90-93° C.

R_{f}=0.98 (CH₂Cl₂/MeOH=15:1 (v/v), staining: CAM).

¹H NMR (300 MHz, CDCl₃): δ=8.09-7.92 (m, 3H, 2×Ph-H and 1×Py-H), 7.87-7.77 (m, 2H, Py-H), 7.44 (s, 1H, SCHC), 7.38-7.28 (m, 1H, SCHCH), 7.27-7.16 (m, 1H, SCHCH), 6.99 (d, ³J=8.8 Hz, 2H, Ph-H), 5.46 (s, 2H, CO₂CH₂), 4.09 (q, ³J=6.9 Hz, 2H, CH₂, CH₃), 1.44 (t, ³J=7.0 Hz, 3H, CH₃).

¹³C NMR (76 MHz, CDCl₃): δ=165.4, 160.4, 157.5, 148.0, 137.6, 136.8, 131.0, 128.6 (2C), 127.9, 126.3, 124.8, 122.9, 122.8, 114.8 (2C), 63.7, 62.5, 14.9.

HRMS (DI-EI): Calcd. m/z for C₁₉H₁₇NO₃S: 339.0929; found: 339.0925.

### Example 198: Furan-3-ylmethyl 6-(4-ethoxyphenyl)picolinate

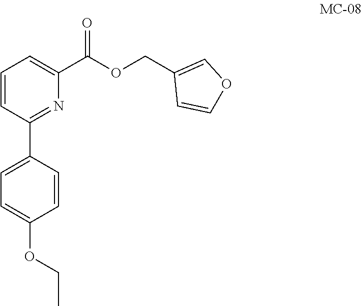 [SMILES: CCOc1ccc(-c2cccc(C(=O)OCc3ccoc3)n2)cc1]

According to general procedure D; 76.2 μL (0.884 mmol, 3.0 eq) 3-furanmethanol, 18 h reaction time. Purification was performed via flash column chromatography (15 g SiO₂, cyclohexane/EtOAc 10:1 (v/v), column size 10×2 cm).

Yield: 52.6 mg (0.163 mmol, 55%), pale yellow solid.

C₁₉H₁₇NO₄ [323.35 g/mol].

mp=97-101° C.

R_{f}=0.98 (CH₂Cl₂/MeOH=15:1 (v/v), staining: CAM).

¹H NMR (300 MHz, CDCl₃): δ=8.07-7.91 (m, 3H, 2×Ph-H and 1×Py-H), 7.86-7.77 (m, 2H, Py-H), 7.60 (s, 1H, OCHC), 7.42 (s, 1H, OCHCH), 6.98 (d, ³J=8.5 Hz, 2H, Ph-H), 6.56 (s, 1H, OCHCH), 5.32 (s, 2H, CO₂CH₂), 4.09 (q, ³J=6.8 Hz, 2H, CH₂CH₃), 1.44 (t, ³J=6.8 Hz, 3H, CH₃).

¹³C NMR (76 MHz, CDCl₃): δ=165.5, 160.4, 157.5, 148.0, 143.5, 142.1, 137.6, 131.0, 128.6 (2C), 122.9, 122.8, 120.4, 114.8 (2C), 111.0, 63.7, 59.0, 14.9.

HRMS (DI-EI): Calcd. m/z for C₁₉H₁₇NO₄: 323.1158; found: 323.1157.

### Example 199: Thiazol-5-ylmethyl 6-(4-ethoxyphenyl)picolinate

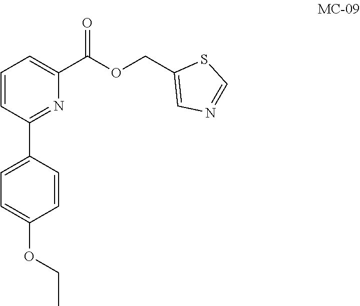 [SMILES: CCOc1ccc(-c2cccc(C(=O)OCc3cncs3)n2)cc1]

According to general procedure D; 77.1 μL (0.884 mmol, 3.0 eq) 5-thiazolemethanol, 16 h reaction time. Purification was performed via flash column chromatography (10 g SiO₂, cyclohexane/EtOAc 10:1→1:1 (v/v), column size 7×2 cm), followed by preparative RP-HPLC (method B).

Yield: 44.7 mg (0.131 mmol, 45%), pale yellow solid.

C₁₈H₁₆N₂O₃S [340.40 g/mol].

mp=75-80° C.

R_{f}=0.98 (CH₂Cl₂/MeOH=15:1 (v/v), staining: CAM).

¹H NMR (300 MHz, CDCl₃): δ=8.84 (s, 1H, SCHN), 8.08-7.91 (m, 4H, 2×Ph-H, 1×Ar—H from thiazole, 1×Py-H), 7.88-7.77 (m, 2H, Py-H), 6.98 (d, 3J=8.5 Hz, 2H, Ph-H), 5.65 (s, 2H, CO₂CH₂), 4.08 (q, ³J=6.8 Hz, 2H, CH₂CH₃), 1.43 (t, ³J=6.8 Hz, 3H, CH₃).

¹³C NMR (76 MHz, CDCl₃): δ=165.3, 160.5, 157.6, 155.0, 147.4, 144.3, 137.7, 132.6, 130.8, 128.6 (2C), 123.2, 122.9, 114.9 (2C), 63.7, 58.9, 14.9.

HRMS (DI-EI): Calcd. m/z for C₁₈H₁₆N₂O₃S: 340.0882; found: 340.0885.

### Example 200: Methyl 6-(4-isopropoxyphenyl)-4-methoxypicolinate

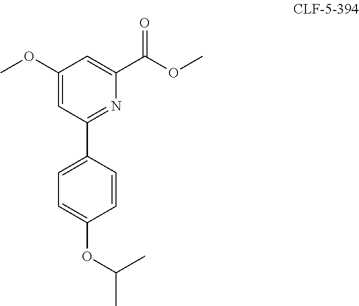

An inert 500 mL round bottom flask with Schienk adapter and a magnetic stirring bar was charged with a solution of 3.00 g (14.9 mmol, 1.0 eq) methyl 6-chloro-4-methoxypicolinate in 175 mL degassed DME abs. 4-Isopropoxyphenylboronic acid (3.48 g, 19.3 mmol, 1.3 eq), cesium fluoride (4.75 g, 31.2 mmol, 2.1 eq) and 540 mg (0.744 mmol, 5.0 mol-%) PdCl₂(dppf) were added subsequently and the red suspension was heated to 80° C. (oil bath). After 21 h the reaction mixture was cooled to RT, filtered through a pad of Celite® and the filter cake was washed with EtOAc (4×50 mL). The solvent was removed under reduced pressure and the crude product was dried in oil-pump vacuum. Purification via flash column chromatography (570 g SiO₂, cyclohexane/EtOAc 4:1 (v/v), column size 26×7.5 cm) yielded the product as a pale-yellow solid.

Yield: 3.93 g (13.0 mmol, 88%), colorless solid.

mp=87-90° C.

C₁₇H₁₉NO₄ [301.34 g/mol].

R_{f}=0.28 (cyclohexane/EtOAc=4:1 (v/v); staining: KMnO₄).

¹H NMR (300 MHz, CDCl₃): δ=7.94 (d, ³J=8.7 Hz, 2H, Ph-H), 7.54 (d, 4J=2.1 Hz, 1H, Py-H), 7.29 (d, 4J=2.1 Hz, 1H, Py-H), 6.95 (d, ³J=8.7 Hz, 2H, Ph-H), 4.62 (hept, ³J=6.0 Hz, 1H, CH(CH₃)₂), 4.00 (s, 3H, CO₂CH₃), 3.94 (s, 3H, OCH₃), 1.34 (d, ³J=6.0 Hz, 6H, CH(CH₃)₂).

¹³C NMR (76 MHz, CDCl₃): δ=167.3, 166.3, 159.3, 159.2, 149.6, 131.1, 128.7 (2C), 116.1 (2C), 109.1, 109.0, 70.1, 55.7, 53.0, 22.1.

### Intermediate 6-(4-isopropoxyphenyl)-4-methoxypicolinic acid

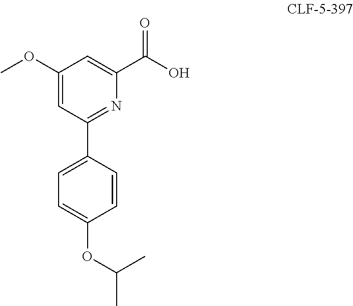 [SMILES: COc1cc(C(=O)O)nc(-c2ccc(OC(C)C)cc2)c1]

To the vigorously stirred solution of CLF-5-394 (3.93 g, 13.0 mmol, 1.0 eq) in 43 mL THF a solution of 2.19 g (52.2 mmol, 4.0 eq) LiOH×H₂O in 65 mL H₂O was added at RT. After 20 min the pale-yellow solution was acidified to pH=2 with 6 M HCl. The mixture was transferred into a separation funnel and the product extracted with EtOAc (3×110 mL). The phases were separated, the combined organic phases dried over Na₂SO₄, filtered, and the solvent was removed under reduced pressure. The crude product was dried in oil-pump vacuum and directly used without further purification.

Yield: 3.75 g (13.0 mmol, quant.), colorless solid.

C₁₆H₁₇NO₄ [287.32 g/mol].

### Example 201: Isopropyl 6-(4-isopropoxyphenyl)-4-methoxypicolinate

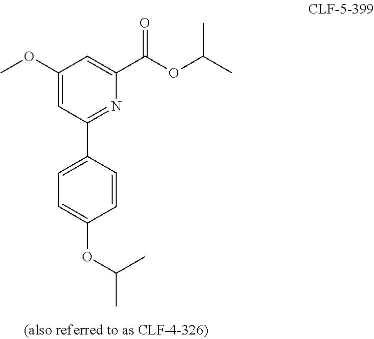 [SMILES: COc1cc(C(=O)OC(C)C)nc(-c2ccc(OC(C)C)cc2)c1]

An inert 250 mL round bottom flask with a Schlenk adapter and a magnetic stirring bar was charged with a solution of CLF-5-397 (3.75 g, 31.0 mmol, 1.0 eq) and 5.0 mL (65 mmol, 5.0 eq) 2-propanol in 70 mL CH₂Cl₂ abs. The solution was cooled to 0° C. and DMAP (159 mg, 1.30 mmol, 0.1 eq), followed by 3.75 g (19.6 mmol, 1.5 eq) of EDC hydrochloride were added. The ice bath was removed and the pale-yellow solution was left to spontaneous warmup overnight. After 15 h reaction time the mixture was diluted with 70 mL CH₂Cl₂, transferred into a separation funnel and washed with H₂O (2×70 mL). The phases were separated and the org. phase was evaporated. The crude product was dried in oil-pump vacuum and purified via flash column chromatography (550 g SiO₂, cyclohexane/EtOAc 4:1 (v/v), column size 24×7.5 cm).

Yield: 3.89 g (11.8 mmol, 91%), colorless solid.

mp=70-71° C.

C₁₉H₂₃NO₄ [329.40 g/mol].

R_{f}=0.40 (cyclohexane/EtOAc=4:1 (v/v); staining: KMnO₄).

¹H NMR (300 MHz, CDCl₃): δ=7.99 (d, ³J=8.8 Hz, 2H, Ph-H), 7.51 (d, ⁴J=2.1 Hz, 1H, Py-H), 7.30 (d, ⁴J=2.1 Hz, 1H, Py-H), 6.96 (d, ³J=8.7 Hz, 2H, Ph-H), 5.31 (hept, ³J=6.2 Hz, 1H, CO₂CH(CH₃)₂), 4.62 (hept, ³J=5.6 Hz, 1H, OCH(CH₃)₂), 3.95 (s, 3H, CO₂CH₃), 3.94 (s, 3H, OCH₃), 1.43 (d, ³J=6.2 Hz, 6H, CO₂CH(CH₃), 1.36 (d, ³J=6.0 Hz, 6H, OCH(CH₃)₂).

¹³C NMR (76 MHz, CDCl₃): δ=167.2, 165.2, 159.3, 159.0, 150.3, 131.2, 128.7 (2C), 116.0 (2C), 108.95, 109.0, 70.1, 69.6, 55.6, 22.1, 22.0.

HRMS (DI-EI): Calcd. m/z for C₁₉H₂₃NO₄: 329.1627; found: 329.1628.

### Example 202: sec-Butyl 6-(4-Isopropoxyphenyl)-4-methoxypicolinate

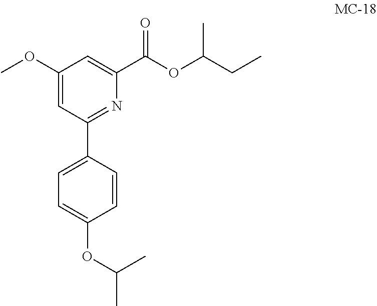 [SMILES: CCC(C)OC(=O)c1cc(OC)cc(-c2ccc(OC(C)C)cc2)n1]

According to general procedure E; 38.2 μL (0.418 mmol, 3.0 eq) butan-2-ol, 16 h reaction time. Purification was performed via flash column chromatography (8 g SiO₂, cyclohexane/EtOAc 8:1 (v/v), column size 10×1.5 cm).

Yield: 25.0 mg (72.8 μmol, 53%), beige solid.

C₂₀H₂₅NO₄ [343.42 g/mol].

mp=68° C.

R_{f}=0.93 (CH₂Cl₂/MeOH=9:1 (v/v)+AcOH, staining: KMnO₄).

¹H NMR (300 MHz, CDCl₃): δ=7.99 (d, ³J=8.7 Hz, 2H, Ph-H), 7.50 (d, ⁴J=2.0 Hz, 1H, Py-H), 7.30 (d, ⁴J=2.0 Hz, 1H, Py-H), 6.96 (d, ³J=8.7 Hz, 2H, Ph-H), 5.22-5.06 (m, 1H, CHCH₂), 4.62 (hept, 3J=6.0 Hz, 1H, CH(CH₃)₂), 3.94 (s, 3H, OCH₃), 1.91-1.62 (m, 2H, CH₂), 1.43-1.30 (m, 9H, 3×CH₃), 1.01 (t, ³J=7.4 Hz, 3H, CH₂CH₃).

¹³C NMR (76 MHz, CDCl₃): δ=167.2, 165.3, 159.3, 159.0, 150.3, 131.2, 128.7 (2C), 116.0 (2C), 109.0, 108.2, 74.0, 70.1, 55.6, 29.0, 22.1 (2C), 19.6, 9.9.

HRMS (DI-EI): Calcd. m/z for C₂₀H₂₅NO₄: 343.1783; found: 343.1781.

### Example 203: Cyclobutyl 6-(4-isopropoxyphenyl)-4-methoxypicolinate

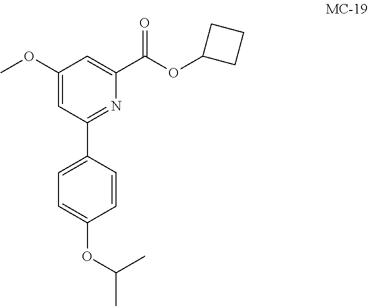 [SMILES: COc1cc(C(=O)OC2CCC2)nc(-c2ccc(OC(C)C)cc2)c1]

According to general procedure E; 32.7 μL (0.418 mmol, 3.0 eq) cyclobutanol, 3 h reaction time. Purification was performed via flash column chromatography (6 g SiO₂, cyclohexane/EtOAc 8:1 (v/v), column size 22×1 cm), followed by preparative RP-HPLC (method C).

Yield: 29.8 mg (87.3 μmol, 63%), colorless solid.

C₂₀H₂₃NO₄ [341.41 g/mol].

mp=116-118° C.

R_{f}=0.96 (CH₂Cl₂/MeOH=9:1 (v/v)+AcOH, staining: KMnO₄).

¹H NMR (300 MHz, CDCl₃): δ=7.98 (d, ³J=8.8 Hz, 2H, Ph-H), 7.53 (d, ⁴J=2.1 Hz, 1H, Py-H), 7.30 (d, ⁴J=2.1 Hz, 1H, Py-H), 6.96 (d, ³J=8.7 Hz, 2H, Ph-H), 5.32-5.17 (m, 1H, CH), 4.62 (hept, ³J=6.0 Hz, 1H, CH(CH₃)₂), 3.94 (s, 3H, OCH₃), 2.58-2.22 (m, 4H, CH₂), 1.96-1.62 (m, 2H, CH₂), 1.36 (d, ³J=6.0 Hz, 6H, CH(CH₃)₂).

¹³C NMR (76 MHz, CDCl₃): δ=167.3, 165.1, 159.3, 159.0, 149.8, 131.1, 128.7 (2C), 116.0 (2C), 108.9, 108.7, 70.4, 70.1, 55.6, 30.5 (2C), 22.1 (2C), 13.8.

HRMS (DI-EI): Calcd. m/z for C₂₀H₂₃NO₄: 341.1627; found: 341.1633.

### Example 204: Propan-2-yl-d₇ 6-(4-isopropoxyphenyl)-4-methoxypicolinate

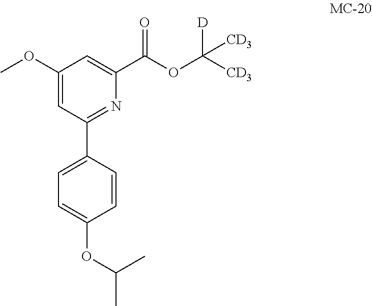 [SMILES: [2H]C([2H])([2H])C([2H])(OC(=O)c1cc(OC)cc(-c2ccc(OC(C)C)cc2)n1)C([2H])([2H])[2H]]

According to general procedure E; 32.0 μL (0.418 mmol, 3.0 eq) 2-propanol-d₈, 3 h reaction time. Purification was performed via flash column chromatography (6 g SiO₂, cyclohexane/EtOAc 5:1 (v/v), column size 22×1 cm), followed by preparative RP-HPLC (method C).

Yield: 32.1 mg (95.4 μmol, 69%), colorless solid.

C₁₉H₁₆D₇NO₄ [336.44 g/mol].

mp=70-72° C.

R_{f}=0.85 (CH₂Cl₂/MeOH=9:1 (v/v)+AcOH, staining: KMnO₄).

¹H NMR (300 MHz, CDCl₃): δ=7.99 (d, ³J=8.7 Hz, 2H, Ph-H), 7.50 (d, ³J=2.0 Hz, 1H, Py-H), 7.29 (d, ⁴J=2.0 Hz, 1H, Py-H), 6.96 (d, ³J=8.7 Hz, 2H, Ph-H), 4.62 (hept, ³J=6.0 Hz, 1H, CH(CH₃)₂), 3.94 (s, 3H, OCH₃), 1.36 (d, ³J=6.0 Hz, 6H, CH(CH₃)₂).

¹³C NMR (76 MHz, CDCl₃): δ=167.2, 165.2, 159.3, 159.0, 150.3, 131.2, 128.7 (2C), 116.0 (2C), 108.9, 108.4, 70.1, 55.6, 22.1 (2C), CO₂CD and 2×CD₃ not visible.

HRMS (DI-EI): Calcd. m/z for C₁₉H₁₆D₇NO₄: 336.2066; found: 336.2079.

### Intermediate 6-Chloro-4-methoxypicolinic acid

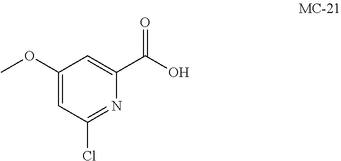

250 mg (1.24 mmol, 1.0 eq) methyl 6-chloro-4-methoxypicolinate were dissolved in 4.1 mL THF in a 25 mL round bottom flask with magnetic stirring bar and a solution of 208 mg (4.96 mmol, 4.0 eq) LiOH×H₂O in 6.2 mL H₂O was added under vigorous stirring at RT. After 30 min the pale yellow solution was acidified to pH=2 with 4 M HCl. The product was extracted with EtOAc (3×13 mL). The organic phase was dried over Na₂SO₄, filtered, and the volatiles were removed under reduced pressure. The colorless solid was dried in oil-pump vacuum and used without further purification.

Yield: 228 mg (1.22 mmol, 98%), colorless solid.

C₇H₆ClNO₃ [187.58 g/mol].

### Intermediate Isopropyl 6-chloro-4-methoxypicolinate

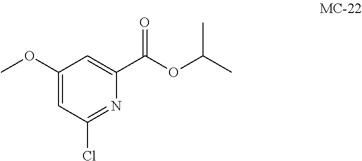

A 30 mL Schlenk tube with magnetic stirring bar was dried with a heatgun, evacuated and purged with Ar. The Schlenk tube was charged with 228 mg (1.22 mmol, 1.0 eq) 6-chloro-4-methoxypicolinic acid (MC-21) and 6.0 mL CH₂Cl₂ abs. The suspension was cooled to 0° C. (ice bath) and subsequently 0.932 μL (12.2 mmol, 10.0 eq) propan-2-ol, 14.9 mg (0.122 mmol, 0.1 eq) DMAP and 350 mg (1.83 mmol, 1.5 eq) EDC.HCl were added. The reaction mixture was stirred and left to spontaneous warm up to RT. After 3 h the pale yellow suspension was diluted with 18.0 mL CH₂Cl₂, washed with H₂O (3×12 mL) and the phases separated. The organic phase was dried over Na₂SO₄, filtered, and the solvent was removed under reduced pressure. The brown oil was purified via flash column chromatography (25 g SiO₂, column size 7×3 cm, cyclohexane/EtOAc=5:1 (v/v)).

Yield: 216 mg (0.941 mmol, 77%) beige solid.

C₁₀H₁₂ClNO₃ [229.66 g/mol].

mp=60-62° C.

R_{f}=0.33 (cyclohexane/EtOAc=5:1 (v/v), staining: KMnO₄).

¹H NMR (300 MHz, CDCl₃): δ=7.51 (d, ⁴J=2.0 Hz, 1H, Ar—H), 6.93 (d, ⁴J=2.0 Hz, 1H, Ar—H), 5.24 (hept, ³J=6.3 Hz, 1H, CH(CH₃)₂), 3.88 (s, 3H, OCH₃), 1.36 (d, ³J=6.3 Hz, 6H, CH(CH₃)₂).

¹³C NMR (76 MHz, CDCl₃): δ=167.9, 163.5, 152.7, 149.8, 112.3, 111.5, 70.1, 56.1, 21.8 (2C).

### Example 205: Isopropyl 6-(4-ethylphenyl)-4-methoxypicolinate

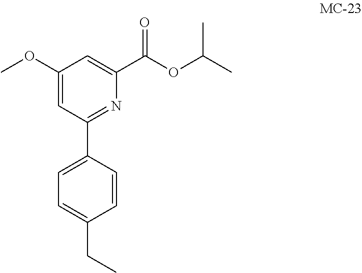 [SMILES: CCc1ccc(-c2cc(OC)cc(C(=O)OC(C)C)n2)cc1]

Isopropyl 6-chloro-4-methoxypicolinate (MC-22) (72.8 mg, 0.317 mmol, 1.0 eq), 4-ethylphenylboronic acid (54.9 mg, 0.366 mmol, 1.2 eq) and cesium fluoride (97.2 mg, 64.0 μmol, 2.1 eq) in 2.7 mL DME abs. were reacted in presence of PdCl₂(dppf) (11.2 mg, 15.3 μmol, 5.0 mol-%) for 5 d at 80° C. according to general procedure B. The crude product was purified via flash column chromatography (18 g SiO₂, cyclohexane/EtOAc=5:1 (v/v), column size column size 12×2 cm), followed by preparative RP-HPLC (method C).

Yield: 39.3 mg (0.131 mmol, 41%), colorless solid.

C₁₈H₂₁NO₃ [299.37 g/mol].

mp=51° C.

R_{f}=0.42 (cyclohexane/EtOAc=5:1 (v/v), staining: KMnO₄).

¹H NMR (300 MHz, CDCl₃): δ=7.97 (d, ³J=8.1 Hz, 2H, Ph-H), 7.54 (d, 4J=2.1 Hz, 1H, Py-H), 7.37-7.23 (m, 3H, 1×Py-H and 2×Ph-H), 5.31 (hept, ³J=6.3 Hz, 1H, CH(CH₃)₂), 3.94 (s, 3H, OCH₃), 2.70 (q, ³J=7.5 Hz, 2H, CH₂), 1.43 (d, ³J=6.2 Hz, 6H, CH(CH₃)₂), 1.26 (t, ³J=7.6 Hz, 3H, CH₂CH₃).

¹³C NMR (76 MHz, CDCl₃): δ=167.2, 165.1, 159.3, 150.4, 145.9, 136.3, 128.3 (2C), 127.3 (2C), 109.3, 109.0, 69.6, 55.6, 28.8, 22.0 (2C), 15.6.

HRMS (DI-EI): Calcd. m/z for C₁₅H₂₁NO₃: 299.1521; found: 299.1528.

### Example 206: Isopropyl 4-methoxy-6-(4-methoxyphenyl)picolinate

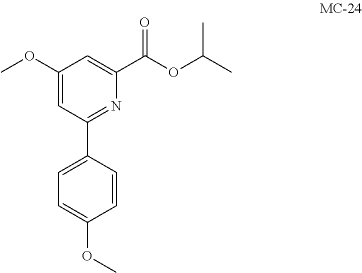 [SMILES: COc1ccc(-c2cc(OC)cc(C(=O)OC(C)C)n2)cc1]

Isopropyl 6-chloro-4-methoxypicolinate (MC-22) (69.6 mg, 0.303 mmol, 1.0 eq), 4-methoxyphenylboronic acid (55.6 mg, 0.366 mmol, 1.2 eq) and cesium fluoride (97.2 mg, 64.0 μmol, 2.1 en) in 2.8 mL DME abs. were reacted in presence of PdCl₂ddppf) 11.2 mg. 15.3 μmol, 5.0 mol-%) for 5 d at 80° C. according to general procedure B. The crude product was purified via flash column chromatography (13 g SiO₂, cyclohexane/EtOAc=5:1 (v/v), column size column size 7×2.3 cm), followed by preparative RP-HPLC (method C).

Yield: 36.6 mg (0.121 mmol, 40%) colorless solid.

C₁₇H₁₉NO₄ [301.34 g/mol].

mp=59-61° C.

R_{f}=0.24 (cyclohexane/EtOAc=5:1 (v/v), staining: KMnO₄).

¹H NMR (300 MHz, CDCl₃): δ=7.94 (d, ³J=8.8 Hz, 2H, Ph-H), 7.43 (d, ⁴J=2.1 Hz, 1H, Py-H), 7.22 (d, ⁴J=2.1 Hz, 1H, Py-H), 5.23 (hept, ³J=6.2 Hz, 1H, CH(CH₃)₂), 3.86 (s, 3H, Py-OCH₃), 3.77 (s, 3H, Ph-OCH₃), 1.35 (d, ³J=6.3 Hz, 6H, CH(CH₃)₂).

¹³C NMR (76 MHz, CDCl₃): δ=167.2, 165.1, 160.9, 158.9, 150.3, 131.4, 128.6 (2C), 114.1 (2C), 109.0, 108.4, 69.5, 55.6, 55.4, 22.0 (2C).

HRMS (DI-EI): Calcd. m/z for C₁₇H₁₉NO₄: 301.1314; found: 301.1320.

### Examples 207 to 236

General Procedures

Reversed Phase Preparative HPLC (Prep HPLC)

Reversed phase preparative HPLC purifications were performed on a Thermo Scientific UltiMate 3000 system. Detection was accomplished with a Dionex UltiMate Diode Array detector. The separations were carried out on a Macherey-Nagel 125/21 Nucleodur® 100-5 C18EC (125×21 mm, 5.0 μm) column. As eluents MeCN and water with 0.05% CF₃COOH as additive was used. Following methods were applied:
Method E 0.0 min-17.0 min linear increase 2 to 100% MeCN, 17.0-19.0 min 100% MeCN isocratic, 19.0-22.0 min linear decrease 100 to 2% MeCN, 22.0-24.0 min 2% MeCN isocratic, 12 mL/min, 30° C.

General Procedure C: Suzuki Coupling

In an inert Schienk flask equipped with magnetic stirring bar heterocyclic bromide (1.0 eq.), boronic acid (0.9 to 1.5 eq.) and K₂CO₃ (2.0 eq.) were dissolved in degassed abs. toluene (0.1 M). Pd[PPh₃]₄ (4 mol %) was added and the reaction mixture was stirred at 80° C. The reaction was monitored via TLC. When full conversion was observed, the reaction mixture was cooled down to RT and filtered through a pad of Celite. The solvent was removed under reduced pressure and the crude product was purified via column chromatography or preparative HPLC, respectively.

General Procedure C: DCC-Mediated Esterification

In an inert 10 mL Schienk flask 2-(4-ethoxyphenyl)thiazole acid (1.0 eq.) was dissolved in abs. DCM (0.1 M). DMAP (0.2 eq.) and the corresponding alcohol (1.5 eq.) were added successively and the reaction mixture was cooled to 0° C. using an ice bath. DCC (1.5 eq.) was added and the cloudy reaction mixture was stirred at RT until full conversion was observed via TLC. The reaction mixture was filtered through a pad of Celite and the solvent was removed under reduced pressure. The crude product was purified via column chromatography or preparative HPLC, respectively.

General Procedure D: Ullmann-Type Coupling

In an inert 10 mL Schlenk flask tert-butyl 5-chloro-2-(4-ethoxyphenyl)thiazole-4-carboxylate (1.0 eq.), CuI (0.1 eq.) and K₂C03 (2.0 eq.) were dissolved in abs. DMF (0.1 M). The corresponding amine (2.1 eq.) was added and the reaction mixture was stirred at 100° C. until full conversion was observed via TLC. The reaction mixture was quenched via the addition of sat. NH₄Cl and extracted with EA. The combined organic layers were dried over Na₂SO₄, filtered and the solvent was removed under reduced pressure. The crude product was purified via column chromatography or preparative HPLC, respectively.

### Example 207: Naphthalen-2-ylmethyl 2-(4-ethoxyphenyl)thiazole-4-carboxylate (LS-13/19)

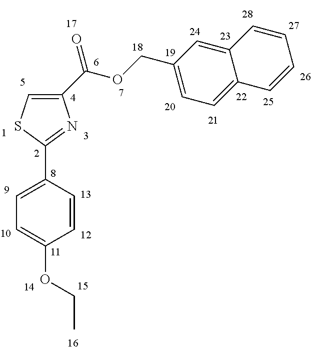 [SMILES: CCOc1ccc(-c2nc(C(=O)OCc3ccc4ccccc4c3)cs2)cc1]

According to general procedure C, 2-(4-ethoxyphenyl)thiazole-4-carboxylic acid (1.0 eq., 401 μmol, 100 mg) was esterified with 2-naphthalenemethanol (1.5 eq., 602 μmol, 95 mg). The crude product was purified via column chromatography (38 g SiO₂, eluent CH/EA 4.5:1).

Yield: 30 mg (78 μmol, 19%) colorless solid

C₂₃H₁₉NO₃S [389.47]

m.p.: 135-138° C.

R_{f}: 0.37 (CH/EA 4:1)

HR-MS [EI, M⁺]: calcd. 389.1086, found 389.1089.

¹H-NMR (300 MHz, CDCl₃): δ (ppm) 8.11 (s, 1H, H5), 8.04-7.73 (m, 6H, H9/13/21/24/25/28), 7.65-7.42 (m, 3H, H20/26/27), 6.94 (d, J=8.6 Hz, 2H, H10/12), 5.58 (s, 2H, H18), 4.09 (q, J=13.8, 6.9 Hz, 2H, H15), 1.44 (t, J=6.9 Hz, 3H, H16).

¹³C-NMR (75.5 MHz, CDCl₃): δ (ppm) 169.1 (C_{q}, C2), 161.5 (C_{q}, C6), 161.3 (C_{q}, C6), 147.6 (C_{q}, C4), 133.4 (2C, C_{q}, C19, C23), 128.7 (2C, CH, C9/13), 128.6, 128.2, 127.9, 127.9 (4 CH, C21/24/25/28), 126.8 (CH, C5), 126.5 (2C, CH, C26/27), 126.3 (CH, C20), 125.7 (C_{q}, C22), 114.9 (2C, CH, C10/12), 67.3 (CH₂, C18), 63.9 (CH₂, C15), 14.9 (CH₃, C16).

### Example 208: Pont-4-yn-2-yl 2-(4-ethoxyphenyl)thiazole-4-carboxylate (LS-20/26)

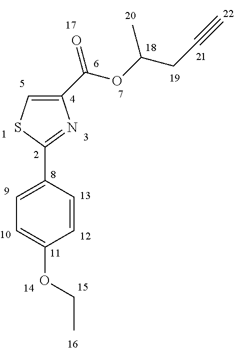 [SMILES: C#CCC(C)OC(=O)c1csc(-c2ccc(OCC)cc2)n1]

According to general procedure C, 2-(4-ethoxyphenyl)thiazole-4-carboxylic acid (1.0 eq., 802 μmol, 200 mg) was esterified with 4-pentyn-2-ol (1.5 eq., 1.20 mmol, 114 μL). The crude product was purified via column chromatography (30 g SiO₂, eluent CH/EA 7:1) and preparative HPLC (method E).

Yield: 121 mg (384 μmol, 48%) colorless oil

C₁₇H₁₇NO₃S [315.39]

R_{f}: 0.23 (CH/EA 5:1)

HR-MS [EI, M⁺]: calcd. 315.0929, found 315.0926.

¹H-NMR (300 MHz, CDCl₃): δ (ppm) 8.07 (s, 1H, H5), 7.93 (d, J=8.8 Hz, 2H, H9/13), 6.94 (d, J=8.7 Hz, 2H, H10/12), 5.29 (q, J=12.1, 6.2 Hz, 1H, H18), 4.09 (q, J=7.0 Hz, 2H, H15), 2.67-2.51 (m, 2H, H19), 2.04 (s, 1H, H22), 1.50 (d, J=6.3 Hz, 3H, H20), 1.44 (t, J=7.0 Hz, 3H, H16).

¹³C-NMR (75.5 MHz, CDCl₃): δ (ppm) 169.1 (C_{q}, C2), 161.3 (C_{q}, C11), 160.8 (C_{q}, C6), 147.7 (C_{q}, C4), 128.7 (2C, CH, C9/13), 126.6 (CH, C5), 125.6 (C_{q}, C8), 114.9 (2C, CH, C10/12), 79.8 (C_{q}, C21), 70.8 (CH, C22), 69.9 (CH, C18), 63.8 (CH₂, C15), 25.8 (CH₂, C19), 19.3 (CH₃, C20), 14.9 (CH₃, C16).

### Example 209: But-2-yn-1-yl 2-(4-ethoxyphenyl)thiazole-4-carboxylate (LS-27)

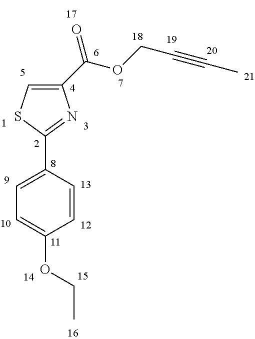 [SMILES: CC#CCOC(=O)c1csc(-c2ccc(OCC)cc2)n1]

According to general procedure C, 2-(4-ethoxyphenyl)thiazole-4-carboxylic acid (1.0 eq., 401 μmol, 100 mg) was esterified with 2-butyn-1-ol (1.5 eq., 602 μmol, 45 μL). The crude product was purified via column chromatography (25 g SiO₂, eluent CH/EA 7:1) and preparative HPLC (method E).

Yield: 33 mg (109 μmol, 27%) colorless solid

C₁₆H₁₅NO₃S [301.36]

R_{f}: 0.21 (CH/EA 7:1)

m.p.: 110-114° C.

HR-MS [EI, M⁺]: calcd. 301.0773, found 301.0770.

¹H-NMR (300 MHz, CDCl₃): δ (ppm) 8.17 (s, 1H, H5), 7.97 (d, J=8.7 Hz, 2H, H9/13), 6.97 (d, J=8.7 Hz, 2H, H10/12), 4.96 (d, J=2.3 Hz, 2H, H18), 4.12 (q, J=7.0 Hz, 2H, H15), 1.91 (t, J=2.2 Hz, 3H, H21), 1.47 (t, J=7.0 Hz, 3H, H16).

¹³C-NMR (75.5 MHz, CDCl₃): δ (ppm) 169.1 (C_{q}, C2), 161.3 (C_{q}, C11), 161.0 (C_{q}, C6), 147.2 (C_{q}, C4), 128.7 (2C, CH, C9/13), 127.1 (CH, C5), 125.6 (C_{q}, C8), 114.9 (2C, CH, C10/12), 83.8 (C_{q}, C19), 73.2 (C_{q}, C20), 63.8 (CH₂, C15), 53.8 (CH₂, C18), 14.9 (CH₃, C16), 3.9 (CH₃, C21).

### Example 210: (S)-But-3-yn-2-yl 2-(4-ethoxyphenyl)thiazole-4-carboxylate (LS-30)

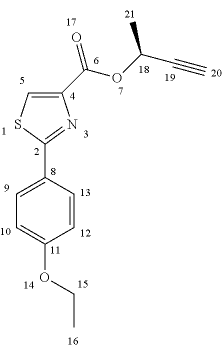 [SMILES: C#C[C@H](C)OC(=O)c1csc(-c2ccc(OCC)cc2)n1]

According to general procedure C, 2-(4-ethoxyphenyl)thiazole-4-carboxylic acid (1.0 eq., 401 μmol, 100 mg) was esterified with (S)-but-3-yn-2-ol (1.5 eq., 602 μmol, 50 μL). The crude product was purified via column chromatography (38 g SiO₂, eluent CH/EA 8:1).

Yield: 86 mg (285 μmol, 70%) colorless solid

C₁₆H₁₅NO₃S [301.36]

R_{f}: 0.25 (CH/EA 8:1)

m.p.: 112-117° C.

HR-MS [EI, M⁺]: calcd. 301.0773, found 301.0769.

¹H-NMR (300 MHz, CDCl₃): δ (ppm) 8.12 (s, 1H, H5), 7.94 (d, J=8.8 Hz, 2H, H9/13), 6.94 (d, J=8.8 Hz, 2H, H10/12), 5.79-5.63 (m, 1H, H18), 4.09 (q, J=6.9 Hz, 2H, H15), 2.51 (d, J=2.1 Hz, 1H, H20), 1.67 (d, J=6.7 Hz, 3H, H21), 1.44 (t, J=7.0 Hz, 3H, H16).

¹³C-NMR (75.5 MHz, CDCl₃): δ (ppm) 169.1 (C_{q}, C2), 161.3 (C11), 160.4 (C_{q}, C2), 147.2 (C_{q}, C4), 128.7 (2C, CH, C9/13), 127.0 (CH, C5), 125.7 (C_{q}, C8), 114.9 (2C, CH, C10/12), 82.0 (C_{q}, C19), 73.6 (CH, C20), 63.8 (CH₂, C15), 61.2 (CH, C18), 21.5 (CH₃, C21), 14.9 (CH₃, C16).

### Example 211: (R)-But-3-yn-2-yl 2-(4-ethoxyphenyl)thiazole-4-carboxylate (LS-31)

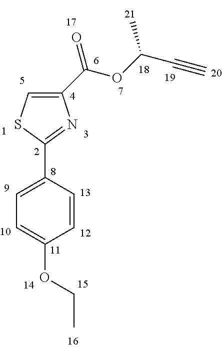 [SMILES: C#C[C@@H](C)OC(=O)c1csc(-c2ccc(OCC)cc2)n1]

According to general procedure C, 2-(4-ethoxyphenyl)thiazole-4-carboxylic acid (1.0 eq., 401 μmol, 100 mg) was esterified with (R)-but-3-yn-2-ol (1.5 eq., 602 μmol, 50 μL). The crude product was purified via column chromatography (38 g SiO₂, eluent CH/EA 8:1).

Yield: 62 mg (206 μmol, 51%) colorless solid

C₁₆H₁₅NO₃S [301.36]

R_{f}: 0.25 (CH/EA 8:1)

m.p.: 120-117° C.

HR-MS [EI, M⁺]: calcd. 301.0773, found 301.0769.

¹H-NMR (300 MHz, CDCl₃): δ (ppm) 8.12 (s, 1H, H5), 7.94 (d, J=8.8 Hz, 2H, H9/13), 6.94 (d, J=8.8 Hz, 2H, H10/12), 5.79-5.63 (m, 1H, H18), 4.09 (q, J=6.9 Hz, 2H, H15), 2.51 (d, J=2.1 Hz, 1H, H20), 1.67 (d, J=6.7 Hz, 3H, H21), 1.44 (t, J=7.0 Hz, 3H, H16).

¹³C-NMR (75.5 MHz, CDCl₃): δ (ppm) 169.1 (C_{q}, C2), 161.3 (C11), 160.4 (C_{q}, C2), 147.2 (C_{q}, C4), 128.7 (2C, CH, C9/13), 127.0 (CH, C5), 125.7 (C_{q}, C8), 114.9 (2C, CH, C10/12), 82.0 (C_{q}, C19), 73.6 (CH, C20), 63.8 (CH₂, C15), 61.2 (CH, C18), 21.5 (CH₃, C21), 14.9 (CH₃, C16).

### Example 212: Butyl 2-(4-ethoxyphenyl)thiazole-4-carboxylate (LS-32)

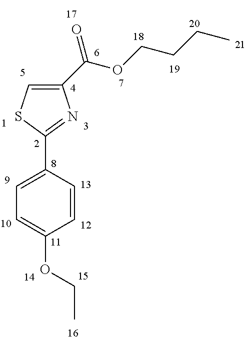 [SMILES: CCCCOC(=O)c1csc(-c2ccc(OCC)cc2)n1]

According to general procedure C, 2-(4-ethoxyphenyl)thiazole-4-carboxylic acid (1.0 eq., 401 μmol, 100 mg) was esterified with 1-butanol (1.5 eq., 602 μmol, 55 μL). The crude product was purified via column chromatography (25 g SiO₂, eluent CH/EA 7:1).

Yield: 103 mg (337 μmol, 84%) colorless solid

C₁₆H₁₉NO₃S [305.39]

R_{f}: 0.42 (CH/EA 4:1)

m.p.: 61-65° C.

HR-MS [EI, M⁺]: calcd. 305.1086, found 305.1082.

¹H-NMR (300 MHz, CDCl₃): δ (ppm) 8.06 (s, 1H, H5), 7.94 (d, J=8.8 Hz, 2H, H9/13), 6.94 (d, J=8.8 Hz, 2H, H10/12), 4.38 (t, J=6.7 Hz, 2H, H18), 4.09 (q, J=6.9 Hz, 2H, H15), 1.85-1.73 (m, 2H, H19), 1.53-1.38 (m, 5H, H16/20), 0.98 (t, J=7.3 Hz, 3H, H16).

¹³C-NMR (75.5 MHz, CDCl₃): δ (ppm) 168.9 (C_{q}, C2), 161.8 (C_{q}, C6), 161.2 (C_{q}, C11), 148.0 (C_{q}, C4), 128.7 (2C, CH, C9/13), 126.2 (CH, C5), 125.8 (C_{q}, C8), 114.9 (2C, CH, C10/12), 65.4 (CH₂, C18), 63.8 (CH₂, C15), 30.9 (CH₂, C19), 19.4 (CH₂, C20), 14.9 (CH₂, C16), 13.9 (CH₃, C21).

### Example 213: 2-(Thiophen-2-yl)ethyl 2-(4-ethoxyphenyl)thiazole-4-carboxylate (LYSU-07)

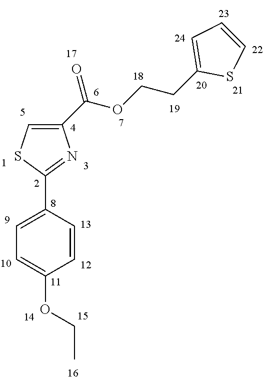 [SMILES: CCOc1ccc(-c2nc(C(=O)OCCc3cccs3)cs2)cc1]

According to general procedure C, 2-(4-ethoxyphenyl)thiazole-4-carboxylic acid (1.0 eq., 401 μmol, 100 mg) was esterified with 2-thiophenethanol (1.5 eq., 602 μmol, 68 μL). The crude product was purified via column chromatography (50 g SiO₂, eluent CH/EA 19:1 to 12:1).

Yield: 140 mg (389 μmol, 97%) colorless solid

C₁₈H₁₇NO₃S₂[359.46]

m.p.: 76-78° C.

R_{f}: 0.52 (CH/EA 4:1)

HR-MS [EI, M⁺]: calcd. 359.0650, found 359.0652.

¹H-NMR (300 MHz, CDCl₃): δ (ppm) 8.09 (s, 1H, H5), 7.94 (d, J=8.7 Hz, 2H, H9/13), 7.18 (d, J=4.9 Hz, 1H, H22), 7.08-6.81 (m, J=5.7, 3.1 Hz, 4H, H10/12/23/24), 4.58 (t, J=6.8 Hz, 2H, H18), 4.09 (q, J=6.9 Hz, 2H, H15), 3.33 (t, J=6.7 Hz, 2H, H19), 1.44 (t, J=7.0 Hz, 3H, H16).

¹³C-NMR (75.5 MHz, CDCl₃): δ (ppm) 169.0 (C_{q}, C2), 161.4 (C_{q}, C6), 161.2 (C_{q}, C11), 147.6 (C_{q}, C4), 139.8 (C_{q}, C20), 128.7 (2C, CH, C9/13), 127.1 (CH; C24), 126.7 (CH, C5), 125.9 (CH, C23), 125.7 (C_{q}, C8), 124.3 (CH, C22), 114.9 (2C, CH, C10/12), 65.6 (CH₂, C18), 63.8 (CH₂, C15), 29.5 (CH₂, C19), 14.9 (CH₃, C16).

### Example 214: 2-(Thiophen-3-yl)ethyl 2-(4-ethoxyphenyl)thiazole-4-carboxylate (LYSU-08)

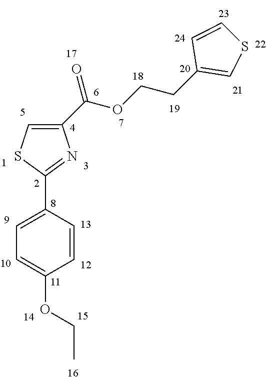 [SMILES: CCOc1ccc(-c2nc(C(=O)OCCc3ccsc3)cs2)cc1]

According to general procedure C, 2-(4-ethoxyphenyl)thiazole-4-carboxylic acid (1.0 eq., 401 μmol, 100 mg) was esterified with 3-thiophenethanol (1.5 eq., 602 μmol, 69 μL). The crude product was purified via column chromatography (50 g SiO₂, eluent CH/EA 19:1 to 12:1 to 9:1).

Yield: 134 mg (373 μmol, 91%) colorless solid

C₁₈H₁₇NO₃S₂[359.46]

m.p.: 76-78° C.

R_{f}: 0.50 (CH/EA 4:1)

HR-MS [EI, M⁺]: calcd. 359.0650, found 359.0653.

¹H-NMR (300 MHz, CDCl₃): δ (ppm) 8.05 (s, 1H, H5), 7.94 (d, J=8.7 Hz, 2H, H9/13), 7.34-7.27 (m, 1H, H24), 7.12 (s, 1H, H21), 7.06 (d, J=4.8 Hz, 1H, H23), 6.95 (d, J=8.7 Hz, 2H, H10/12), 4.57 (t, J=7.0 Hz, 2H, H18), 4.09 (q, J=6.9 Hz, 2H, H15), 3.15 (t, J=7.0 Hz, 2H, H19), 1.44 (t, J=7.0 Hz, 3H, H16).

¹³C-NMR (75.5 MHz, CDCl₃): δ (ppm) 169.0 (C_{q}, C2), 161.5 (C_{q}, C6), 161.2 (C_{q}, C11), 147.7 (C_{q}, C4), 138.0 (C_{q}, C20), 128.7 (2C, CH, C9/13), 128.5 (CH, C23), 126.5 (CH, C5), 125.8 (CH, C24), 125.7 (C_{q}, C8), 121.9 (CH, C21), 114.9 (2C, CH, C10/12), 65.3 (CH₂, C18), 63.8 (CH₂, C15), 29.8 (CH₂, C19), 14.9 (CH₃, C16).

### Example 215: Thiophen-3-ylmethyl 2-(4-ethoxyphenyl)thiazole-4-carboxylate (LYSU-11)

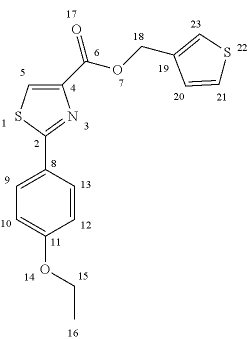 [SMILES: CCOc1ccc(-c2nc(C(=O)OCc3ccsc3)cs2)cc1]

According to general procedure C, 2-(4-ethoxyphenyl)thiazole-4-carboxylic acid (1.0 eq., 401 μmol, 100 mg) was esterified with 3-thiophenemethanol (1.5 eq., 602 μmol, 58 μL). The crude product was purified via column chromatography (50 g SiO₂, eluent CH/EA 19:1 to 17:1 to 15:1 to 13:1).

Yield: 136 mg (394 μmol, 98%) colorless solid

C₁₇H₁₅NO₃S₂ [345.43]

m.p.: 79-81° C.

R_{f}: 0.50 (CH/EA 4:1)

HR-MS [EI, M⁺]: calcd. 345.0493, found 345.0495

¹H-NMR (300 MHz, CDCl₃): δ (ppm) 7.89 (s, 1H, H5), 7.73 (d, J=8.7 Hz, 2H, H9/13), 7.22 (s, J=14.5 Hz, 1H, H23), 7.17-7.08 (m, 1H, H21), 7.01 (d, J=4.8 Hz, 1H, H20), 6.74 (d, J=8.7 Hz, 2H, H10/12), 5.22 (s, 2H, H18), 3.89 (q, J=6.9 Hz, 2H, H15), 1.24 (t, J=6.9 Hz, 3H, H16).

¹³C-NMR (75.5 MHz, CDCl₃): δ (ppm) 169.0 (C_{q}, C2), 161.4 (C_{q}, C6), 161.2 (C_{q}, C11), 147.6 (C_{q}, C4), 136.7 (C_{q}, C19), 128.7 (2C, CH, C9/13), 128.0 (CH, C20), 126.8 (CH, C5), 126.3 (CH, C21), 125.7 (C_{q}, C8), 125.1 (CH, C23), 114.9 (2C, CH, C10/12), 63.8 (CH₂, C15), 62.1 (CH₂, C18), 14.9 (CH₃, C16).

### Example 216: Furan-3-ylmethyl 2-(4-ethoxyphenyl)thiazole-4-carboxylate (LYSU-12)

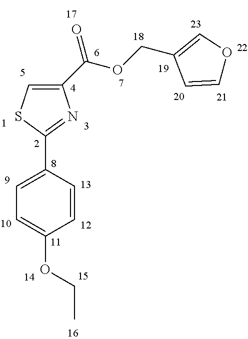 [SMILES: CCOc1ccc(-c2nc(C(=O)OCc3ccoc3)cs2)cc1]

According to general procedure C, 2-(4-ethoxyphenyl)thiazole-4-carboxylic acid (1.0 eq., 401 μmol, 100 mg) was esterified with 3-furanmethanol (1.5 eq., 602 μmol, 54 μL). The crude product was purified via column chromatography (50 g SiO₂, eluent CH/EA 19:1 to 17:1 to 15:1 to 13:1).

Yield: 120 mg (364 μmol, 91%) colorless solid

C₁₇H₁₅NO₄S [329.37]

m.p.: 82-84° C.

R_{f}: 0.45 (CH/EA 4:1)

HR-MS [EI, M⁺]: calcd. 329.0722, found 329.0719.

¹H-NMR (300 MHz, CDCl₃): δ (ppm) 8.01 (s, 1H, H5), 7.86 (d, J=8.7 Hz, 2H, H9/13), 7.51 (s, 1H, H23), 7.34 (s, 1H, H21), 6.87 (d, J=8.7 Hz, 2H, H10/12), 6.47 (s, 1H, H, H20), 5.21 (s, 2H, H18), 4.02 (q, J=6.9 Hz, 2H, H15), 1.37 (t, J=6.9 Hz, 3H, H16).

¹³C-NMR (75.5 MHz, CDCl₃): δ (ppm) 169.1 (C_{q}, C2), 161.5 (C_{q}, C6), 161.3 (C_{q}, C11), 147.6 (C_{q}, C4), 143.5 (CH, C21), 142.3 (CH, C23), 128.7 (2C, CH, C9/13), 126.7 (CH, C5), 125.7 (C_{q}, C8), 120.4 (C_{q}, C19), 114.9 (2C, CH, C10/12), 111.1 (CH, C20), 63.8 (CH₂, C15), 58.6 (CH₂, C18), 14.9 (CH₃, C16).

### Example 217: Thiazol-5-ylmethyl-2-(4-ethoxyphenyl)thiazole-4-carboxylate (LYSU-16)

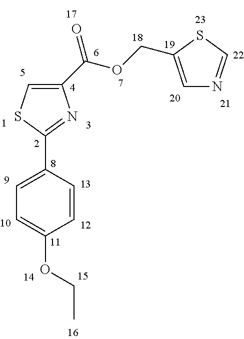 [SMILES: CCOc1ccc(-c2nc(C(=O)OCc3cncs3)cs2)cc1]

According to general procedure C, 2-(4-ethoxyphenyl)thiazole-4-carboxylic acid (1.0 eq., 401 μmol, 100 mg) was esterified with thiazol-5-ylmethanol (1.5 eq., 602 μmol, 55 μL). The crude product was purified via column chromatography (50 g SiO₂, eluent CH/EA 11:1 to 9:1 to 4:1).

Yield: 133 mg (384 μmol, 98%) colorless solid

C₁₆H₁₄N₂O₃S₂ [346.42]

m.p.: 100-102° C.

R_{f}: 0.60 (CH/EA 1:1)

HR-MS [EI, M⁺]: calcd. 346.0446, found 346.0443.

¹H-NMR (300 MHz, CDCl₃): δ (ppm) 8.84 (s, 1H, H22), 8.11 (s, 1H, H5), 8.00 (s, 1H, H20), 7.92 (d, J=8.8 Hz, 2H, H9/13), 6.94 (d, J=8.8 Hz, 2H, H10/12), 5.61 (s, 2H, H18), 4.08 (q, J=6.9 Hz, 2H, H15), 1.44 (t, J=7.0 Hz, 3H, H16).

¹³C-NMR (75.5 MHz, CDCl₃): δ (ppm) 169.3 (C_{q}, C2), 161.3 (C_{q}, C11), 161.2 (C_{q}, C6), 155.1 (CH, C22), 146.9 (C_{q}, C4), 144.5 (CH, C20), 128.7 (2C, CH, C9/13), 127.4 (CH, C5), 125.5 (C_{q}, C8), 114.9 (2C, CH, C10/12), 63.8 (CH₂, C15), 58.5 (CH₂, C18), 14.8 (CH₃, C16).

### Example 218: Oxazol-4-ylmethyl 2-(4-ethoxyphenyl)thiazole-4-carboxylate (AM-5-305)

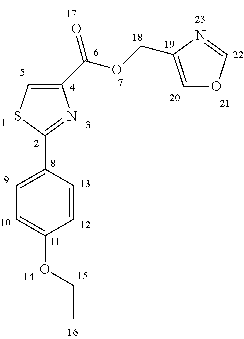 [SMILES: CCOc1ccc(-c2nc(C(=O)OCc3cocn3)cs2)cc1]

According to general procedure C, 2-(4-ethoxyphenyl)thiazole-4-carboxylic acid (1.1 eq., 377 μmol, 94 mg) was esterified with oxazol-4-ylmethanol (1.0 eq., 343 μmol, 34 mg). The crude product was purified via column chromatography (25 g SiO₂, eluent CH/EA 2.5:1) and preparative HPLC (method E).

Yield: 35 mg (106 μmol, 31%) colorless solid

C₁₆H₁₄N₂O₄S [330.36]

m.p.: 121° C.

R_{f}: 0.36 (CH/EA 4:1)

HR-MS [EI, M⁺]: calcd. 330.0674, found 330.0671.

¹H-NMR (300 MHz, CDCl₃): δ (ppm) 8.11 (s, 1H, H5), 8.02-7.87 (m, 3H, H9/13/22), 7.82 (s, 1H, H20), 6.93 (d, J=8.8 Hz, 2H, H10/12), 5.36 (s, 2H, H18), 4.08 (q, J=6.9 Hz, 2H, H15), 1.43 (t, J=7.0 Hz, 3H, H16).

¹³C-NMR (75.5 MHz, CDCl₃): δ (ppm) 169.1 (C_{q}, C2), 161.3 (2C, C_{q}, C6/11), 151.4 (CH, C22), 147.1 (C_{q}, C4), 138.3 (CH, C20), 135.5 (C_{q}, C19), 128.7 (2C, CH, C9/13), 127.1 (CH, C5), 125.6 (C_{q}, C8), 114.0 (2C, CH, C10/12), 63.8 (CH₂, C15), 58.6 (CH₂, C18), 14.9 (CH₃, C16).

### Example 219: (2-Methylthiazol-4-yl)methyl 2-(4-ethoxyphenyl)thiazole-4-carboxylate (AM-5-306a)

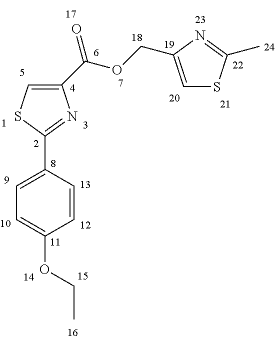 [SMILES: CCOc1ccc(-c2nc(C(=O)OCc3csc(C)n3)cs2)cc1]

According to general procedure C, 2-(4-ethoxyphenyl)thiazole-4-carboxylic acid (1.0 eq., 301 μmol, 75 mg) was esterified with (2-methylthiazol-4-yl)methanol (1.5 eq., 451 μmol, 58 mg). The crude product was purified via column chromatography (20 g SiO₂, eluent CH/EA 2.5:1).

Yield: 89 mg (247 μmol, 82%) colorless solid

C₁₇H₁₆N₂O₃S₂ [360.45]

m.p.: 100° C.

R_{f}:0.50 (CH/EA 1:1)

HR-MS [EI, M⁺]: calcd. 360.0602, found 360.0601.

¹H-NMR (300 MHz, CDCl₃): δ (ppm) 8.12 (s, 1H, H5), 7.93 (d, J=8.8 Hz, 2H, H9/13), 7.26 (s, 1H, H20), 6.93 (d, J=8.8 Hz, 2H, H10/12), 5.46 (s, 2H, H18), 4.08 (q, J=6.9 Hz, 2H, H15), 2.73 (s, 3H, H24), 1.43 (t, J=7.0 Hz, 3H, H16).

¹³C-NMR (75.5 MHz, CDCl₃): δ (ppm) 169.0 (C_{q}, C2), 166.7 (C_{q}, C22), 161.2 (2 C_{q}, C6/11), 150.6 (C_{q}, C19), 147.3 (C_{q}, C4), 128.7 (2C, CH, C9/13), 127.0 (CH, C5), 125.7 (C_{q}, C8), 118.1 (CH, C20), 114.9 (2C, CH, C10/12), 63.8 (CH₂, C15), 62.4 (CH₂, C18), 19.3 (CH₃, C24), 14.9 (CH₃, C16).

### Example 220: Oxazol-5-yl 2-(4-ethoxyphenyl)thiazole-4-carboxylate (AM-5-306b)

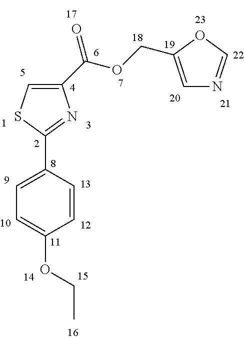 [SMILES: CCOc1ccc(-c2nc(C(=O)OCc3cnco3)cs2)cc1]

According to general procedure C, 2-(4-ethoxyphenyl)thiazole-4-carboxylic acid (1.0 eq., 301 μmol, 75 mg) was esterified with oxazol-5-ylmethanol (1.5 eq., 451 μmol, 48 mg). The crude product was purified via column chromatography (20 g SiO₂, eluent CH/EA 2.5:1).

Yield: 53 mg (160 μmol, 40%) colorless solid

C₁₆H₁₄N₂O₄S [330.36]

m.p.: 116° C.

R_{f}: 0.42 (CH/EA 1:1)

HR-MS [EI, M⁺]: calcd. 330.0674, found 330.0669.

¹H-NMR (300 MHz, CDCl₃): δ (ppm) 8.11 (s, 1H, H5), 7.99-7.78 (m, 3H, H9/13/22), 7.25 (s, J=2.9 Hz, 1H, H20), 6.94 (d, J=8.8 Hz, 2H, H10/12), 5.42 (s, 2H, H18), 4.08 (q, J=7.0 Hz, 2H, H15), 1.43 (t, J=7.0 Hz, 3H, H16).

¹³C-NMR (75.5 MHz, CDCl₃): δ (ppm) 169.3 (C_{q}, C2), 161.3 (C_{q}, C11), 161.0 (C_{q}, C6), 151.9 (C_{q}, C19), 151.9 (CH, C22), 146.8 (C_{q}, C4), 128.7 (2C, CH, C9/13), 127.6 (CH, C20), 127.4 (CH; C5), 125.5 (C_{q}, C8), 114.9 (2C, CH, C10/12), 63.8 (CH₂, C16), 56.1 (CH₂, C18), 14.9 (CH₃, C16).

### Example 221: (5-Methylthiophen-2-yl)methyl 2-(4-ethoxyphenyl)thiazole-4-carboxylate (LS-25)

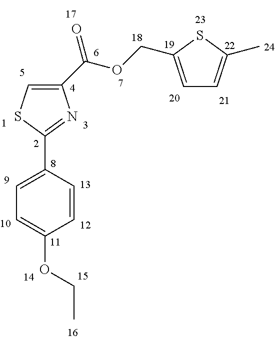 [SMILES: CCOc1ccc(-c2nc(C(=O)OCc3ccc(C)s3)cs2)cc1]

According to general procedure C, 2-(4-ethoxyphenyl)thiazole-4-carboxylic acid (1.0 eq., 401 μmol, 100 mg) was esterified with (5-methylthiopen-2-yl)methanol (1.5 eq., 602 μmol, 77 mg). The crude product was purified via column chromatography (38 g SiO₂, eluent CH/EA 9:1).

Yield: 43 mg (120 μmol, 30%) off-white solid

C₁₈H₁₇NO₃S₂[359.46]

m.p.: 55-58° C.

R_{f}: 0.27 (CH/EA 9:1)

HR-MS [EI, M⁺]: calcd. 359.0650, found 359.0648.

¹H-NMR (300 MHz, CDCl₃): δ (ppm) 8.08 (s, 1H, H5), 7.92 (d, J=8.5 Hz, 2H, H9/13), 7.08-6.82 (m, 3H, H10/12/20), 6.64 (s, 1H, H21), 5.46 (s, 2H, H18), 4.08 (q, J=6.4 Hz, 2H, H15), 2.47 (s, 3H, H24), 1.44 (t, J=6.7 Hz, 3H, H16).

¹³C-NMR (75.5 MHz, CDCl₃): δ (ppm) 169.0 (C_{q}, C2), 161.3 (C_{q}, C6), 161.2 (C_{q}, C11), 147.5 (CH, C4), 142.1 (C_{q}, C19), 135.3 (C_{q}, C22), 129.1 (CH, C20), 128.7 (2C, CH, C9/13), 126.9 (CH, C5), 125.7 (C_{q}, C8), 125.1 (CH, C21), 114.9 (2C, CH, C10/12), 63.8 (CH₂, C15), 61.6 (CH₂, C18), 15.5 (CH₃, C24), 14.9 (CH₃, C16).

### Example 222: (5-Methlyfuran-2-yl)methyl 2-(4-ethoxyphenyl)thiazole-4-carboxylate (LS-34)

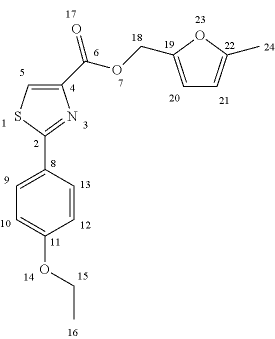 [SMILES: CCOc1ccc(-c2nc(C(=O)OCc3ccc(C)o3)cs2)cc1]

According to general procedure C, 2-(4-ethoxyphenyl)thiazole-4-carboxylic acid (1.0 eq., 401 μmol, 100 mg) was esterified with (5-methylfuran-2-yl)methanol (1.5 eq., 602 μmol, 68 mg). The crude product was purified via column chromatography (25 g SiO₂, eluent CH/EA 7:1).

Yield: 122 mg (356 μmol, 89%) yellowish solid

C₁₈H₁₇NO₃S₂ [343.40]

m.p.: 73-79° C.

R_{f}: 0.33 (CH/EA 4:1)

HR-MS [EI, M⁺]: calcd. 343.0878, found 343.0876.

¹H-NMR (300 MHz, CDCl₃): δ (ppm) 8.09 (s, 1H, H5), 7.93 (d, J=8.6 Hz, 2H, H9/13), 6.93 (d, J=8.6 Hz, 2H, H10/12), 6.39 (d, J=2.5 Hz, 1H, H20), 5.96 (s, 1H, H21), 5.29 (s, 2H, H18), 4.08 (d, J=6.9 Hz, 2H, H15), 2.31 (s, 3H, H24), 1.44 (t, J=6.9 Hz, 3H, H16).

¹³C-NMR (75.5 MHz, CDCl₃): δ (ppm) 169.0 (C_{q}, C2), 161.3 (C_{q}, C11), 161.2 (C_{q}, C6), 153.4 (C_{q}, C19), 147.6 (C_{q}, C4), 147.5 (C_{q}, C22), 128.7 (2C, CH, C9/13), 126.9 (CH, C5), 125.7 (C_{q}, C8), 114.9 (2C, CH, C10/12), 112.5 (CH, C20), 106.8 (CH, C21), 63.8 (CH₂, C15), 59.1 (CH₂, C18), 14.9 (CH₃, C16), 13.8 (CH₃, C24).

### Example 223: (5-Chlorothiophen-2-yl)methyl 2-(4-ethoxyphenyl)thiazole-4-carboxylate (LS-39)

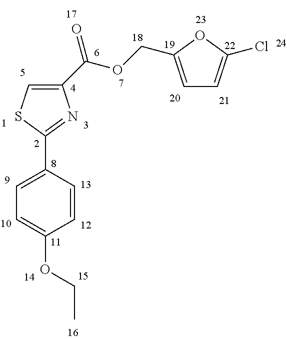 [SMILES: CCOc1ccc(-c2nc(C(=O)OCc3ccc(Cl)o3)cs2)cc1]

According to general procedure C, 2-(4-ethoxyphenyl)thiazole-4-carboxylic acid (1.0 eq., 281 μmol, 70 mg) was esterified with (5-chlorothiophen-2-yl)methanol (1.25 eq., 350 μmol, 52 mg). The crude product was purified via column chromatography (25 g SiO₂, eluent CH/EA 4:1).

Yield: 51 mg (134 μmol, 47%) yellowish solid

C₁₇H₁₄ClNO₃S₂[379.87]

m.p.: 77-81° C.

R_{f}: 0.38 (CH/EA 4:1)

HR-MS [EI, M⁺]: calcd. 379.0104, found 379.0104

¹H-NMR (300 MHz, CDCl₃): δ (ppm) 8.10 (s, 1H, H5), 7.92 (d, J=8.8 Hz, 2H, H9/13), 7.07-6.89 (m, 3H, H10/12/20), 6.81 (d, J=3.7 Hz, 1H, H21), 5.43 (s, 2H, H18), 4.09 (q, J=6.9 Hz, 2H, H15), 1.44 (t, J=6.9 Hz, 3H, H16).

¹³C-NMR (75.5 MHz, CDCl₃): δ (ppm) 169.2 (C_{q}, C2), 161.3 (C_{q}, C11), 161.3 (C_{q}, C6), 147.2 (C_{q}, C4), 136.5 (C_{q}, C19), 131.9 (C_{q}, C22), 128.7 (2C, CH; C9/13), 128.4 (CH, C20), 127.2 (CH, C5), 125.9 (CH, C21), 125.6 (C_{q}, C8), 114.9 (2C, CH; C10/12), 63.8 (CH₂, C15), 61.4 (CH₂, C18), 14.9 (CH₃, C16).

### Example 224: (5-Bromothiophen-2-yl)methyl 2-(4-ethoxyphenyl)thiazole-4-carboxylate (LS-40)

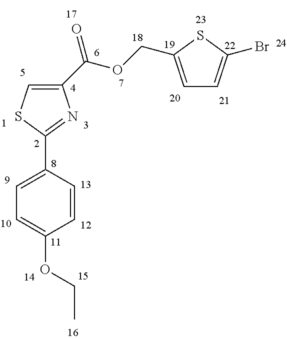 [SMILES: CCOc1ccc(-c2nc(C(=O)OCc3ccc(Br)s3)cs2)cc1]

According to general procedure C, 2-(4-ethoxyphenyl)thiazole-4-carboxylic acid (1.0 eq., 401 μmol, 100 mg) was esterified with (5-bromothiophen-2-yl)methanol (1.25 eq., 500 μmol, 96 mg). The crude product was purified via column chromatography (25 g SiO₂, eluent CH/EA 6:1).

Yield: 148 mg (349 μmol, 87%) yellowish solid

C₁₇H₁₄BrNO₃S₂ [424.33]

m.p.: 73-77° C.

R_{f}: 0.23 (CH/EA 4:1)

HR-MS [EI, M⁺]: calcd. 424.9578, found 424.9576.

¹H-NMR (300 MHz, CDCl₃): δ (ppm) 8.10 (s, 1H, H5), 7.92 (d, J=8.6 Hz, 2H, H9/13), 6.93 (d, J=8.6 Hz, 4H, H10/12/20/21), 5.45 (s, 2H, H18), 4.08 (q, J=6.8 Hz, 2H, H15), 1.43 (t, J=6.9 Hz, 3H, H16).

¹³C-NMR (75.5 MHz, CDCl₃): δ (ppm) 169.2 (C_{q}, C2), 161.4 (C_{q}, C11), 161.2 (C_{q}, C6), 147.1 (C_{q}, C4), 139.4 (C_{q}, C19), 129.7 (CH, C21), 129.3 (CH, C20), 128.7 (2C, CH, C9113), 127.2 (CH, C5), 125.6 (C_{q}, C8), 114.9 (2C, CH, C10/12), 114.2 (C_{q}, C22), 63.8 (CH₂, C15), 61.2 (CH₂, C18), 14.9 (CH₃, C16).

### Example 22: 1-(Thiazol-2-yl)ethyl 2-(4-ethoxyphenyl)thiazole-4-carboxylate (LYSU-15)

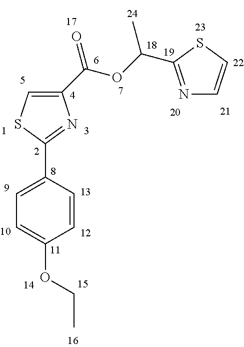 [SMILES: CCOc1ccc(-c2nc(C(=O)OC(C)c3nccs3)cs2)cc1]

According to general procedure C, 2-(4-ethoxyphenyl)thiazole-4-carboxylic acid (1.0 eq., 401 μmol, 100 mg) was esterified with 1-(thiazol-2-yl)ethan-1-ol (1.5 eq., 602 μmol, 79 mg).

The crude product was purified via column chromatography (50 g SiO₂, eluent CH/EA 19:1 to 17:1 to 9:1).

Yield: 144 mg (400 μmol, quant.) colorless solid

C₁₇H₁₆N₂O₃S₂ [360.45]

m.p.: 64-66°

R_{f}: 0.73 (CH/EA 1:1)

HR-MS [EI, M⁺]: calcd. 360.0602, found 360.0605.

¹H-NMR (300 MHz, CDCl₃): δ (ppm) 8.15 (s, 1H, H5), 7.94 (d, J=8.7 Hz, 2H, H9/13), 7.79 (d, J=3.2 Hz, 1H, H21), 7.34 (d, J=3.2 Hz, 1H, H22), 6.94 (d, J=8.8 Hz, 2H, H10/12), 6.46 (q, J=6.5 Hz, 1H, H18), 4.08 (q, J=6.9 Hz, 2H, H15), 1.87 (d, J=6.6 Hz, 3H, H24), 1.44 (t, J=7.0 Hz, 3H, H16).

¹³C-NMR (75.5 MHz, CDCl₃): δ (ppm) 170.1 (C_{q}, C19), 169.2 (C_{q}, C2), 161.3 (C_{q}, C11), 160.3 (C_{q}, C6), 147.1 (C_{q}, C4), 142.9 (CH, C5), 128.7 (2C, CH, C9/13), 127.2 (CH, C21), 125.6 (C_{q}, C8), 119.6 (CH, C22), 114.9 (2C, CH, C10/12), 70.7 (CH, C18), 63.8 (CH₂, C15), 20.9 (CH₃, C24), 14.9 (CH₃, C16).

### Intermediate: Ethyl 2-diazo-3-oxopentanoate (S1a)

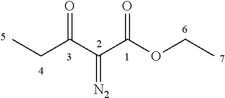

In a 250 mL Schlenk flask, 4-acetamidobenzenesulfonyl azide (1.1 eq., 6.24 mmol, 1.51 g) was dissolved in 40 mL abs. MeCN and cooled down to 0° C. Ethyl 3-oxovalerate (1.0 eq., 5.68 mmol, 810 μL) and Et₃N (3.0 eq., 17 mmol, 2.36 mL) were added dropwise. After 15 min, the cooling bath was removed and the reaction mixture was stirred at RT overnight. When full conversion was observed via TLC, the colorless precipitate was removed by filtration and rinsed with 20 mL Et₂O/pentane (1:1). The solvent was removed under reduced pressure. The yellow oil was diluted with 10 mL Et₂O/pentane (1:1) and the colorless precipitate was again removed by filtration. The solvent was removed on a rotary evaporator, yielding a yellow oil, which was used in the next step without further purification.

Yield: 982 mg (5.77 mmol, 98%) yellow oil

C₇H₁₀N₂O₃[170.17]

R_{f}: 0.50 (CH/EA 4:1)

¹H-NMR (300 MHz, CDCl₃): δ (ppm) 4.23 (q, J=7.1 Hz, 2H, H6), 2.79 (q, J=7.3 Hz, 2H, H4), 1.26 (t, J=7.1 Hz, 3H, H7), 1.07 (t, J=7.3 Hz, 3H, H5).

¹³C-NMR (75.5 MHz, CDCl₃): δ (ppm) 193.7 (C_{q}, C3), 161.6 (C_{q}, C1), 61.5 (CH₂, C6), 33.9 (CH₂, C4), 14.5 (CH₃, C7), 8.4 (CH₃, C5).

### Intermediate: Ethyl 2-diazo-4-methyl-3-oxopentanoate (S1b)

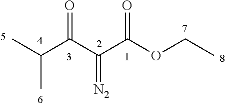

According to the synthesis of ethyl 2-diazo-3-oxopentanoate (S1a), ethyl isoburyrylacetate (1.0 eq., 5.68 mmol, 920 μL) was converted to the diazo-derivative.

Yield: 1.04 g (5.64 mmol, 98%) yellow oil

C₈H₁₂N₂O₃ [184.20]

R_{f}: 0.60 (CH/EA 4:1)

¹H-NMR (300 MHz, CDCl₃): δ (ppm) 4.29 (q, J=7.1 Hz, 2H, H7), 3.66-3.46 (m, J=13.6, 6.8 Hz, 1H, H4), 1.33 (t, J=7.1 Hz, 3H, H8), 1.13 (d, J=6.8 Hz, 6H, H5/6).

¹³C-NMR (75.5 MHz, CDCl₃): δ (ppm) 197.2 (C_{q}, C3), 161.3 (C_{q}, C1), 61.5 (CH₂, C7), 36.9 (CH, C4), 18.7 (2C, CH₃, C5/6), 14.5 (CH₃, C8).

### Intermediate: Ethyl 2-(4-ethoxybenzamido)-3-oxopentanoate (S2a)

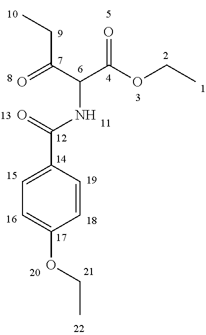 [SMILES: CCOC(=O)C(NC(=O)c1ccc(OCC)cc1)C(=O)CC]

A 50 mL three-necked flask with magnetic stirring bar was equipped with a stopper, a closed air condenser and a stopcock connected to the Schlenk line. The flask was charged with 4-ethoxybenzamide (1.0 eq., 2.42 mmol, 401 mg), Rh₂(OAc)₄ (3 mol %, 28.2 mg) and 5 mL abs. DCE. In a 50 ml round-bottom flask with magnetic stirring bar and Schlenk adapter, 5 mL of a 1.1 M solution of S1a in abs. DCE were prepared. Using a syringe pump, 3.00 mL (1.4 eq., 3.39 mmol) of the 1.1 M solution of S1a were added through a septum within 16 h (rate: 187 μL/h). A stopcock connected to the Schienk line and a bubbler was fitted on top of the air condenser to maintain a continuous argon flow. The valve on the bottom neck was closed as soon as the addition of S1a started. The reaction was heated to 90° C. and stirred overnight. TLC analysis indicated full conversion of the starting material. The brown suspension was transferred into a round bottom flask and the solvent was removed under reduced pressure. The crude product was purified via column chromatography (75 g SiO₂, CH/EA 5:2).

Yield: 539 mg (1.75 mmol, 72%) yellowish solid

C₁₆H₂₁NO₅ [307.35]

m.p.: 68° C.

R_{f}: 0.55 (CH/EA 1:1)

HR-MS [EI, M⁺]: calcd. 307.1420, found 307.1423.

¹H-NMR (300 MHz, CDCl₃): δ (ppm) 7.73 (d, J=8.8 Hz, 2H, H15/19), 7.14 (d, 1H, H11) 6.86 (d, J=8.8 Hz, 2H, H16/18), 5.35 (d, J=6.4 Hz, 1H, H6), 4.23 (q, J=7.1 Hz, 2H, H2), 4.01 (q, J=6.9 Hz, 2H, H21), 2.75 (m, 2H, H9), 1.37 (t, J=6.9 Hz, 3H, H22), 1.25 (t, J=7.1 Hz, 3H, H1), 1.07 (t, J=7.2 Hz, 3H, H10).

¹³C-NMR (75.5 MHz, CDCl₃): δ (ppm) 202.1 (C_{q}, C7), 166.6 (C_{q}, C4), 166.4 (C_{q}, C12), 162.1 (C_{q}, C17), 129.2 (2 CH, C15/19), 125.1 (C_{q}, C14), 114.3 (_{C}_{_{q}}, C16/18), 63.7 (CH₂, C21), 62.7 (CH, C6), 62.6 (CH₂, C2), 34.4 (CH₂, C9), 14.7 (CH₃, C22), 14.1 (CH₃, C1), 7.5 (CH₃, C10)

### Intermediate: Ethyl 2-(4-ethoxybenzamido)-4-methyl-3-oxopentanoate (S2b)

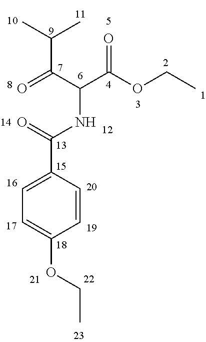 [SMILES: CCOC(=O)C(NC(=O)c1ccc(OCC)cc1)C(=O)C(C)C]

According to the synthesis of S2a, S2b was obtained from ethyl 2-diazo-4-methyl-3-oxopentanoate (S1b) (1.1 M in DCE, 1.4 eq., 3.39 mmol, 3 mL) and 4-ethoxybenzamide (1.0 eq., 2.49 mmol, 412 mg). The product was purified via preparative HPLC (method E).

Yield: 524 mg (1.63 mmol, 65%) yellowish oil

C₁₇H₂₃NO₅ [321.37

R_{f}: 0.45 (CH/EA 1:1)

HR-MS [EI, M⁺]: calcd. 321.1576, found 321.1577.

¹H-NMR (300 MHz, CDCl₃): δ (ppm) 7.73 (d, J=8.8 Hz, 2H, H-16/20), 7.16 (d, J=10.4 Hz, 1H, H-12), 6.85 (d, J=8.8 Hz, 2H, H-17/19), 5.51 (d, J=6.6 Hz, 1H, H-6), 4.27 (q, J=7.1 Hz, 2H, H-2), 4.01 (q, J=5.6 Hz, 2H, H-22), 3.14-2.98 (m, 1H, H-9), 1.36 (m, J=21.4 Hz, 3H, H-23), 1.24 (t, J=7.1 Hz, 3H, H-1), 1.18 (d, J=7.0 Hz, 3H, H-10), 1.08 (d, J=6.7 Hz, 3H, H-11).

¹³C-NMR (75.5 MHz, CDCl₃): δ (ppm) 205.6 (C_{q}, C7), 166.7 (C_{q}, C4), 166.7 (C_{q}, C13), 162.3 (C_{q}, C18), 129.3 (2 CH, C16/20), 125.2 (C_{q}, C15), 114.5 (2 CH, C16/18), 63.9 (CH₂, C22), 62.8 (CH, C6), 61.4 (CH₂, C2), 39.0 (CH, C9), 19.0-17.8 (CH₃, C10/11), 14.8 (CH₃, C23), 14.2 (CH₃, C1).

### Example 226: Ethyl 2-(4-ethoxyphenyl)-5-ethylthiazole-4-carboxylate (RE-26a)

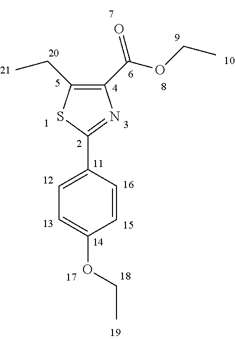 [SMILES: CCOC(=O)c1nc(-c2ccc(OCC)cc2)sc1CC]

In a 50 mL round bottom flask equipped with magnetic stirring bar and Schienk adapter, ethyl 2-(4-ethoxybenzamido)-3-oxopentanoate (S2a) (1.0 eq., 1.56 mmol, 480 mg) was dissolved in 15 mL abs. THF. Lawesson's reagent (2.0 eq., 3.13 mmol, 1.27 g) was added and the reaction was stirred at 70° C. overnight. When full conversion was observed via TLC, the reaction mixture was cooled down to RT and the solvent was removed under reduced pressure. The crude product was purified via column chromatography (50 g SiO₂, CH/EA 6:1).

Yield: 135 mg (442 μmol, 28%) yellowish solid

C₁₈H₁₉NO₃S [305.39]

m.p.: 74-78° C.

R_{f}: 0.31 (CH/EA 1:1)

HR-MS [EI, M⁺]: calcd. 305.1086, found 305.1091.

¹H-NMR (300 MHz, CDCl₃): δ (ppm) 7.86 (d, J=8.8 Hz, 2H, H12/18), 6.92 (d, J=8.8 Hz, 2H, H13/15), 4.43 (d, J=7.1 Hz, 2H, H9), 4.08 (q, J=6.9 Hz, 2H, H18), 3.26 (q, J=7.4 Hz, 2H, H20), 1.43 (t, J=7.0 Hz, 6H, H10/19), 1.36 (t, J=7.5 Hz, 3H, H21).

¹³C-NMR (75.5 MHz, CDCl₃): δ (ppm) 164.0 (Cq, C2), 162.8 (Cq, C6), 160.8 (Cq, C14), 151.6 (Cq, C5), 141.2 (Cq, C4), 128.3 (2C, CH, C12/16), 126.0 (Cq, C11), 114.8 (2C, CH, C13/15), 63.8 (CH2, C18), 61.2 (CH2, C9), 21.5 (CH2, C20), 16.2 (CH3, C21), 14.9 (CH3, C19), 14.5 (CH3, C10).

### Example 227: Ethyl 2-(4-ethoxyphenyl)-5-isopropylthiazole-4-carboxylate (RE-26b)

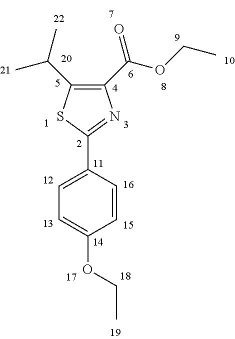 [SMILES: CCOC(=O)c1nc(-c2ccc(OCC)cc2)sc1C(C)C]

In a 50 mL round bottom flask equipped with magnetic stirring bar and Schlenk adapter, ethyl 2-(4-ethoxybenzamido)-4-methyl-3-oxopentanoate (S2b) (1.0 eq., 1.40 mmol, 450 mg) was dissolved in 15 mL abs. THF. Lawesson's reagent (2.0 eq., 2.80 mmol, 1.13 g) was added and the reaction was stirred at 70° C. overnight. When full conversion was observed via TLC, the reaction mixture was cooled down to RT and the solvent was removed under reduced pressure. The crude product was purified via column chromatography (75 g SiO₂, CH/EA 6:1).

Yield: 124 mg (388 μmol, 28%) yellow solid

C₁₇H₂₁NO₃S [319.39]

m.p.: 78-84° C.

R_{f}: 0.31 (CH/EA 1:1)

HR-MS [EI, M⁺]: calcd. 319.1242, found 319.1244.

¹H-NMR (300 MHz, CDCl₃): δ (ppm) 7.86 (d, J=8.8 Hz, 2H, H12/16), 6.92 (d, J=8.7 Hz, 2H, H13/15), 4.43 (d, J=7.1 Hz, 2H, H9), 4.15-3.99 (m, 3H, H18/20), 1.44 (t, J=7.1 Hz, 6H, H10/19), 1.37 (d, J=6.8 Hz, 6H, H21/22).

¹³C-NMR (75.5 MHz, CDCl₃): δ (ppm) 163.9 (Cq, C2), 162.8 (Cq, C6), 160.8 (Cq, C14), 157.9 (Cq, C4), 140.5 (Cq C5), 128.3 (2C, CH, C12/16), 126.1 (Cq, C11), 114.8 (2C, CH, C13/15), 63.8 (CH2, C18), 61.3 (CH2, C9), 28.2 (CH, C20), 25.3 (2C, CH3, C21/22), 14.9 (CH3, C19), 14.5 (CH3, C10).

### Example 228: Ethyl 5-butoxy-2-(4-ethoxyphenyl)thiazole-4-carboxylate (AM-4-292)

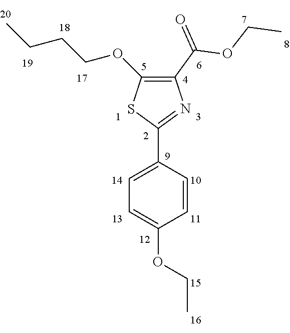 [SMILES: CCCCOc1sc(-c2ccc(OCC)cc2)nc1C(=O)OCC]

In an inert Schlenk flask, NaH (60% in mineral oil, 3.1 eq., 1.45 mmol, 58 mg) was suspended in 5 mL abs. dioxane. Ethyl 5-chloro-2-(4-ethoxyphenyl)thiazole-4-carboxylate (1.0 eq., 0.48 mmol, 150 mg) and n-butanol (3.0 eq., 1.44 mmol, 132 μL) were added. The flask was equipped with a bubbler and the reaction was stirred at 80° C. for 2 d. After full conversion was observed via TLC, the reaction was quenched via addition of 5 mL H₂O. The reaction mixture was acidified to pH=2 and extracted with 2×10 mL EA. The solvent was removed under reduced pressure and the residue was taken up in 15 mL EtOH. H₂SO₄ (80 μL) was added and the mixture was stirred at 80° C. overnight. The solvent was again removed under reduced pressure, the residue was taken up in EA and washed with 1×10 mL sat. NaHCO₃. The organic layer was dried over Na₂SO₄, filtered and the solvent was removed under reduced pressure. The crude product was purified via column chromatography (13 g SiO₂, eluent 8:1 to 6:1) and preparative HPLC (method E).

Yield: 36 mg (140 μmol, 22%) colorless solid

C₁₈H₂₃NO₄S [349.45]

m.p. 62° C.

R_{f}: 0.35 (CH/EA 4:1)

HR-MS [EI, M⁺]: calcd. 349.1348, found 349.1346.

¹H-NMR (300 MHz, CDCl₃): δ (ppm) 7.70 (d, J=8.7 Hz, 2H; H10/14), 6.83 (d, J=8.7 Hz, 2H, H11/13), 4.33 (q, J=7.1 Hz, 2H; H7), 4.15 (t, J=6.4 Hz, 2H, H17), 3.99 (q, J=6.9 Hz, 2H, H15), 1.89-1.71 (m, 2H, H18), 1.56-1.41 (m, 2H, H19), 1.34 (q, J=6.9 Hz, 6H, H8/16), 0.92 (t, J=7.4 Hz, 3H, H20).

¹³C-NMR (75.5 MHz, CDCl₃): δ (ppm) 168.1 (C_{q}, C2), 161.9 (C_{q}, C11), 160.6 (C_{q}, C6), 152.9 (C_{q}, C4), 127.7 (2C, CH, C10/14), 127.2 (C_{q}, C9), 126.2 (C_{q}, C5), 114.8 (2C, CH, C11/13), 78.5 (CH₂, C17), 63.7 (CH₂, C15), 60.8 (CH₂, C7), 31.4 (CH₂, C18), 19.1 (CH₂, C19), 14.9 (CH₃, C16), 14.5 (CH₃, C8), 13.8 (CH₃, C20).

### Example 229: tert-Butyl 2-(4-ethoxyphenyl)-5-morpholinothiazole-4-carboxylate (AM-4-293)

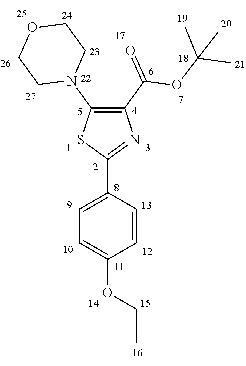 [SMILES: CCOc1ccc(-c2nc(C(=O)OC(C)(C)C)c(N3CCOCC3)s2)cc1]

According to general procedure D, tert-butyl 5-chloro-2-(4-ethoxyphenyl)thiazole-4-carboxylate (1.0 eq., 88 μL, 30 mg) was coupled with morpholine (2.1 eq., 185 μmol, 15 μL). As no conversion could be observed after 20 h, additional 10 mg CuI (0.6 eq.,) and 30 μL morpholine (4.2 eq.) were added and the temperature was raised to 110° C. The reaction was stirred for further 17 h. The crude product was purified via column chromatography (8 g SiO₂, eluent CH/EA 5:1).

Yield: 20 mg (51 μmol, 58%) yellowish solid

C₂₀H₂₆N₂O₄S [390.50]

m.p.: 127° C.

R_{f}: 0.35 (CH/EA 4:1)

HR-MS [EI, M⁺]: calcd. 390.1613, found 390.1616.

¹H-NMR (300 MHz, CDCl₃): δ (ppm) 7.80 (d, J=8.6 Hz, 2H, H9/13), 6.90 (d, J=8.6 Hz, 2H, H10/12), 4.06 (q, J=13.9, 6.9 Hz, 2H, H15), 3.88 (d, J=4.4 Hz, 4H, 24/26), 3.21 (d, J=4.3 Hz, 4H, H23/27), 1.63 (s, 9H, H19/20/21), 1.43 (t, J=6.9 Hz, 3H, H16).

¹³C-NMR (75.5 MHz, CDCl₃): (ppm) δ 161.6 (C_{q}, C2), 161.1 (C_{q}, C11), 160.5 (C_{q}, C6), 155.9 (C_{q}, C5), 132.0 (C_{q}, C5), 127.8 (2C, CH, C9/13), 126.4 (C_{q}, C8), 114.8 (2C, CH, C10/12), 81.6 (C_{q}, C18), 66.6 (2C, CH₂, C24/26), 63.7 (CH₂, C15), 54.5 (2C, CH₂, C23127), 28.6 (3C, CH₃, C19/20/21), 14.9 (CH₃, C16).

### Example 230: 1,1,1-Trifluoro-3-methylbutan-2-yl 2-(4-ethoxyphenyl)thiazole-4-carboxylate (AM-3-220a)

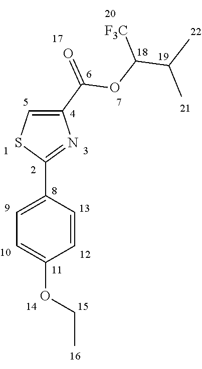 [SMILES: CCOc1ccc(-c2nc(C(=O)OC(C(C)C)C(F)(F)F)cs2)cc1]

According to general procedure C, 2-(4-ethoxyphenyl)thiazole-4-carboxylic acid (1.0 eq., 401 μmol, 100 mg) was esterified with 1,1,1,-trifluoro-3-methylbutan-2-ol (1.5 eq., 602 μmol, 75 μL). The crude product was purified via column chromatography (25 g SiO₂, eluent CH/EA 12:1).

Yield: 93 mg (249 μmol, 62%) colorless solid

C₁₇H₁₈F₃NO₃S [373.39]

m.p.: 96° C.

R_{f}: 0.47 (CH/EA 4:1)

HR-MS [EI, M⁺]: calcd. 373.0959, found 373.0963.

¹H-NMR (300 MHz, CDCl₃): δ (ppm) 8.15 (s, 1H, H5), 7.95 (d, J=8.7 Hz, 2H, H9/13), 6.95 (d, J=8.7 Hz, 2H, H10/12), 5.42 (dt, J=14.1, 7.2 Hz, 1H, H18), 4.09 (q, J=6.9 Hz, 2H, H15), 2.31 (td, J=13.1, 6.5 Hz, 1H, H19), 1.44 (t, J=6.9 Hz, 3H, H16), 1.10 (d, J=6.5 Hz, 6H, H21/22).

¹³C-NMR (75.5 MHz, CDCl₃): δ (ppm) 169.4 (C_{q}, C2), 161.4 (C_{q}, C11), 159.8 (C_{q}, C6), 146.2 (C_{q}, C4), 128.7 (2C, CH, C9/13), 127.6 (CH, C5), 125.5 (C_{q}, C8), 115.0 (2C, CH, C10/12), 74.1 (q, CH, C18), 63.9 (CH₂, C15), 28.2 (CH, C19), 19.2 (CH₃, C21), 17.6 (CH₃, C22), 14.9 (CH₃, C16).

### Intermediate: Thiophen-3-ylmethyl 2-bromothiazole-4-carboxylate (S4)

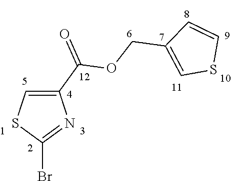 [SMILES: O=C(OCc1ccsc1)c1csc(Br)n1]

According to general procedure C, 2-bromothiazole-4-carboxylic acid (1.0 eq., 4.81 mmol, 1.0 g) was esterified with 3-thiophenemethanol (1.5 eq., 7.21 mmol, 680 μL). The crude product was purified via column chromatography (125 g SiO₂, eluent CH/EA 8:1).

Yield: 1.22 g (4.01 mmol, 84%) colorless solid

C₉H₆BrNO₂S₂ [304.18]

m.p.: 107° C.

R_{f}: 0.53 (CH/EA 4:1)

¹H-NMR (300 MHz, CDCl₃): δ (ppm) 8.12 (s, 1H, H5), 7.40 (s, 1H, H11), 7.31 (dd, J=4.6, 2.7 Hz, 1H, H9), 7.17 (d, J=3.9 Hz, 1H, H8), 5.39 (s, 2H, H6).

¹³C-NMR (75.5 MHz, CDCl₃): δ (ppm) 160.0 (C_{q}, C12), 147.1 (C_{q}, C4), 137.1 (C_{q}, C7), 136.3 (C_{q}, C2), 131.3 (CH, C5), 128.1 (CH, C8), 126.4 (CH, C9), 125.5 (CH, C11), 62.4 CH₂, C6).

### Example 231: Thiophen-3-ylmethyl 2-(4-methoxyphenyl)thiazole-4-carboxylate (AM-5-309a)

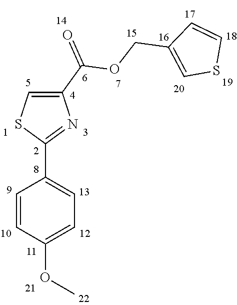 [SMILES: COc1ccc(-c2nc(C(=O)OCc3ccsc3)cs2)cc1]

According to general procedure A, thiophen-3-ylmethyl 2-bromothiazole-4-carboxylate (S4) (1.0 eq., 395 μmol, 120 mg) was coupled with 4-methoxyphenylboronic acid (1.2 eq., 473 μmol, 72 mg). The crude product was purified twice via column chromatography (25 g SiO₂, eluent CH/EA 6:1 and 13 g SiO₂, eluent toluene/EA 20:1).

Yield: 62 mg (187 μmol, 47%) colorless solid

C₁₆H₁₃NO₃S₂[331.40]

m.p.: 91° C.

R_{f}: 0.39 (CH/EA 4:1)

¹H-NMR (300 MHz, CDCl₃): δ (ppm) 8.09 (s, 1H, H5), 7.94 (d, J=8.8 Hz, 2H, H9/13), 7.42 (d, J=1.8 Hz, 1H, H20), 7.32 (dd, J=4.9, 3.0 Hz, 1H, H18), 7.20 (dd, J=4.8, 0.9 Hz, 1H, H17), 6.95 (d, J=8.8 Hz, 2H, H10/12), 5.41 (s, 2H, H15), 3.86 (s, 3H, H22).

¹³C-NMR (75.5 MHz, CDCl₃): δ (ppm) 169.0 (C_{q}, C2), 161.8 (C_{q}, C11), 161.4 (C_{q}, C6), 147.6 (C_{q}, C4), 136.7 (C_{q}, C16), 128.7 (2 CH, C9/13), 128.0 (CH, C17), 126.8 (CH, C5), 126.3 (CH, C18), 125.9 (C_{q}, C8), 125.1 (CH, C20), 114.4 (2 CH, C10/12), 62.9 (CH₂, C15), 55.6 (CH₃, C22).

### Example 232: Thiophen-3-ylmethyl 2-(4-propoxyphenyl)thiazole-4-carboxylate (AM-5-309b)

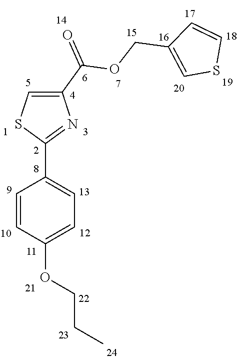 [SMILES: CCCOc1ccc(-c2nc(C(=O)OCc3ccsc3)cs2)cc1]

According to general procedure A, thiophen-3-ylmethyl 2-bromothiazole-4-carboxylate (1.0 eq., 395 μmol, 120 mg) was coupled with 4-propoxyphenylboronic acid (1.2 eq., 473 μmol, 85 mg). The crude product was purified twice via column chromatography (38 g SiO₂, eluent CH/EA 4:1 and 13 g SiO₂, eluent toluene/EA 20:1).

Yield: 74 mg (206 μmol, 52%) colorless solid

C₁₈H₁₇NO₃S₂ [359.46]

m.p.: 89° C.

R_{f}: 0.49 (CH/EA 4:1)

¹H-NMR (300 MHz, CDCl₃): δ (ppm) 8.08 (s, 1H), 7.93 (d, J=8.8 Hz, 2H), 7.41 (s, 1H), 7.32 (dd, J=4.9, 3.0 Hz, 1H), 7.20 (d, J=3.9 Hz, 1H), 6.94 (d, J=8.8 Hz, 2H), 5.41 (s, 2H), 3.97 (t, J=6.5 Hz, 2H), 1.93-1.75 (m, 2H), 1.05 (t, J=7.4 Hz, 3H).

¹³C-NMR (75.5 MHz, CDCl₃): δ (ppm) 169.1, 161.5, 161.4, 147.6, 136.7, 128.7, 128.0, 126.7, 126.3, 125.6, 125.1, 114.9, 69.8, 62.1, 22.7, 10.6.

### Example 233: Thiophen-3-ylmethyl 2-(4-Isopropoxyphenyl)thiazole-4-carboxylate (AM-5-309c)

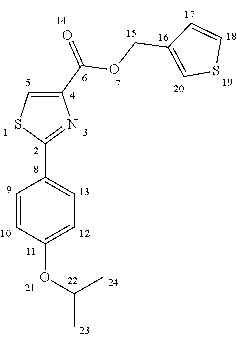 [SMILES: CC(C)Oc1ccc(-c2nc(C(=O)OCc3ccsc3)cs2)cc1]

According to general procedure A, thiophen-3-ylmethyl 2-bromothiazole-4-carboxylate (1.5 eq., 395 μmol, 120 mg) was coupled with 4-isopropoxyphenylboronic acid (1.0 eq., 256 μmol, 46 mg). The crude product was purified via column chromatography (25 g SiO₂, eluent CH/EA 8:1).

Yield: 75 mg (209 μmol, 81%) colorless solid

C₁₈H₁₇NO₃S₂ [359.46]

m.p.: 93° C.

R_{f}: 0.49 (CH/EA 4:1)

¹H-NMR (300 MHz, CDCl₃): δ (ppm) 8.08 (s, 1H), 7.92 (d, J=8.8 Hz, 2H), 7.41 (s, 1H), 7.32 (dd, J=4.8, 3.0 Hz, 1H), 7.20 (d, J=3.9 Hz, 1H), 6.92 (d, J=8.8 Hz, 2H), 5.41 (s, 2H), 4.68-4.55 (m, J=12.0, 6.0 Hz, 1H), 1.36 (d, J=6.0 Hz, 6H).

¹³C-NMR (75.5 MHz, CDCl₃): δ (ppm) 169.1, 161.4, 160.3, 147.6, 136.7, 128.7, 128.0, 126.7, 126.3, 125.5, 125.1, 116.0, 70.2, 62.1, 22.1.

### Example 234: Thiophen-3-ylmethyl 2-(4-(tert-butyl)phenyl)thiazole-4-carboxylate (AM-5-309d)

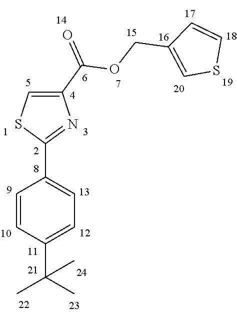 [SMILES: CC(C)(C)c1ccc(-c2nc(C(=O)OCc3ccsc3)cs2)cc1]

According to general procedure A, thiophen-3-ylmethyl 2-bromothiazole-4-carboxylate (1.0 eq., 395 μmol, 120 mg) was coupled with 4-tert-butylphenylboronic acid (1.2 eq., 473 μmol, 84 mg). The crude product was purified via column chromatography (25 g SiO₂, eluent CH/EA 12:1).

Yield: 66 mg (185 μmol, 47%) colorless solid

C₁₉H₁₉NO₂S₂ [357.49]

m.p.: 131° C.

R_{f}: 0.59 (CH/EA 4:1)

¹H-NMR (300 MHz, CDCl₃): δ (ppm) 8.13 (s, 1H), 7.93 (d, J=8.5 Hz, 2H), 7.46 (d, J=8.5 Hz, 2H), 7.42 (s, 1H), 7.33 (dd, J=4.8, 3.0 Hz, 1H), 7.21 (d, J=3.9 Hz, 1H), 5.42 (s, 2H), 1.35 (s, 9H).

¹³C-NMR (75.5 MHz, CDCl₃): δ (ppm) 169.2, 161.4, 154.4, 147.7, 136.7, 130.2, 128.0, 127.2, 126.9, 126.3, 126.1, 125.1, 62.1, 35.1, 31.3.

### Example 235: Thiophen-3-ylmethyl 2-(4-(dimethylamino)phenyl)thiazole-4-carboxylate (AM-5-309e)

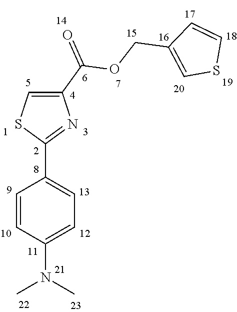 [SMILES: CN(C)c1ccc(-c2nc(C(=O)OCc3ccsc3)cs2)cc1]

According to general procedure A, thiophen-3-ylmethyl 2-bromothiazole-4-carboxylate (1.0 eq., 395 μmol, 120 mg) was coupled with 4-dimethylaminophenylboronic acid (1.2 eq., 473 μmol, 78 mg). The crude product was purified via column chromatography (25 g SiO₂, eluent CH/EA 6:1).

Yield: 77 mg (224 μmol, 57%) colorless solid

C₁₇H₁₆N₂O₂S₂ [344.45]

m.p.: 159° C.

R_{f}: 0.49 (CH/EA 4:1)

¹H-NMR (300 MHz, CDCl₃): δ (ppm) 8.01 (s, 1H), 7.87 (d, J=8.9 Hz, 2H), 7.41 (s, 1H), 7.32 (dd, J=4.9, 3.0 Hz, 1H), 7.20 (d, J=3.9 Hz, 1H), 6.70 (d, J=8.9 Hz, 2H), 5.41 (s, 2H), 3.03 (s, 6H).

¹³C-NMR (75.5 MHz, CDCl₃): δ (ppm) 169.9, 161.6, 152.1, 147.3, 136.8, 128.4, 128.0, 126.3, 125.7, 125.0, 121.1, 111.8, 62.0, 40.3.

### Example 236: (2-(4-Ethoxyphenyl)thiazol-4-yl)methyl 2-(4-ethoxyphenyl)thiazole-4-carboxylate (AM-4-302)

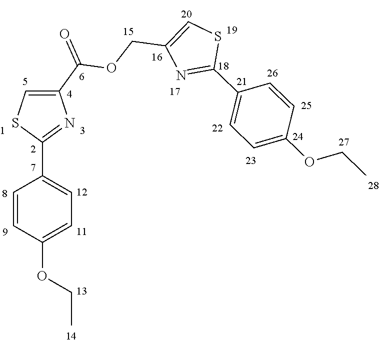 [SMILES: CCOc1ccc(-c2nc(COC(=O)c3csc(-c4ccc(OCC)cc4)n3)cs2)cc1]

According to general procedure C, 2-(4-ethoxyphenyl)thiazole-4-carboxylic acid (1.0 eq., 225 μmol, 56 mg) was esterified with (2-(4-ethoxyphenyl)thiazol-4-yl)methanol (1.3 eq., 292 μmol, 69 mg). The crude product was purified via column chromatography (25 g SiO₂, eluent CH/EA 4:1).

Yield: 93 mg (249 μmol, 62%) colorless solid

C₁₇H₁₈F₃NO₃S [373.39]

R_{f}: 0.76 (CH/EA 1:1)

¹H-NMR (300 MHz, CDCl₃): δ (ppm) 8.14 (s, 1H), 7.91 (dd, J=16.1, 8.8 Hz, 4H), 7.33 (s, 1H), 6.93 (d, J=8.7 Hz, 4H), 5.54 (s, 2H), 4.08 (q, J=6.9 Hz, 4H), 1.43 (t, J=6.9 Hz, 6H).

¹³C-NMR (75.5 MHz, CDCl₃): δ (ppm) 169.0, 168.8, 161.3, 161.2, 160.8, 151.7, 147.4, 128.7, 128.3, 127.0, 126.3, 125.7, 117.2, 114.9, 63.8, 63.8, 62.7, 14.8.

### Example 237: Biological Experiments (I)

Determination of IC₅₀ Values of Human ATGL Inhibitors

Triglyceride (TG) hydrolase activity was measured as previously described (Schweiger M et al., Methods in Enzymology, 2014, 538:171-93). In brief, TG hydrolase activity was determined by adding recombinant purified CGI-58 to Expi cell lysates expressing human ATGL. For determination of cross-species reactivity Expi lysates expressing ATGL from different model organisms (e.g. mouse, rat, rhesus monkey, goat, etc.) were used. Samples were incubated with DMSO (carrier) or increasing concentrations of ATGL inhibitors dissolved in DMSO. TG substrate was prepared by emulsifying 330 μM radiolabelled triolein (40,000 c.p.m./nmol) and 45 μM phosphatidylcholine/phosphatidylinositol (3:1) in 100 mM potassium phosphate buffer (pH 7.0) by sonication and adjusting to 5% essentially FA-free BSA. TG substrate was added to the reaction mixture and incubated for 1 h at 37° C. in the water bath. The reaction was terminated by adding methanol/chloroform/heptane (10:9:7) and 0.1 M potassium carbonate with 0.1 M boric acid (pH 10.5). After centrifugation at 800 g for 15 min the radioactivity in the upper phase was determined by liquid scintillation counting and the rate of FA hydrolysis was calculated. IC₅₀ values were determined as inhibitor concentration capable of inhibiting TG hydrolase activity by 50% as compared to DMSO control. Samples were measured as duplicates. The determination of IC₅₀ values for human ATGL is illustrated in FIG. 1.

The IC₅₀ values of a range of exemplary compounds of formula (I) against human ATGL as well as their inhibitory effect on murine ATGL (in % inhibition at 50 μM compound) are summarized in the following table:

| | | Inhibition | Inhibition of |
|---|---|---|---|
| | | of human | murine ATGL |
| Compound | | ATGL − IC₅₀ [μM] | at 50 μM − mean [%] |
| Example 2 | NG-385 | 20 μM | 5.9% |
| Example 6 | NG-399 | 3 μM | 0.0% |
| Example 8 | NG-402 | 10 μM | 9.1% |
| Example 11 | NG-416 | 5 μM | 1.8% |
| Example 12 | NG-417 | 25 μM | 0.0% |
| Example 13 | NG-418 | 8 μM | |
| Example 14 | NG-423 | 10 μM | 6.1% |
| Example 15 | NG-427 | 6 μM | 0.0% |
| Example 16 | NG-428 | 3 μM | 18.1% |
| Example 18 | NG-433 | 10 μM | 0.0% |
| Example 19 | NG-434 | 3 μM | 3.9% |
| Example 20 | NG-441 | 4 μM | |
| Example 21 | NG-445 | 5 μM | 0.0% |
| Example 23 | NG-451 | 15 μM | 16.0% |
| Example 24 | NG-460 | 25 μM | 38.0% |
| Example 25 | NG-466 | 8 μM | 10.9% |
| Example 26 | NG-470 | 12 μM | 23.1% |
| Example 27 | NG-474 | 3 μM | 0.0% |
| Example 28 | NG-480 | 12 μM | 0.0% |
| Example 29 | NG-487 | 6 μM | 0.0% |
| Example 30 | NG-488 | 8 μM | 0.0% |
| Example 31 | NG-489 | 12 μM | 6.0% |
| Example 32 | NG-490 | 6 μM | 18.6% |
| Example 35 | NG-497 | 1 μM | 0.0% |
| Example 36 | NG-510 | 8 μM | 9.6% |
| Example 37 | NG-512 | 10 μM | 22.8% |
| Example 38 | NG-513 | 5 μM | 5.7% |
| Example 40 | NG-530 | 10 μM | |
| Example 41 | NG-531 | 3 μM | 28.2% |
| Example 42 | NG-534 | 10 μM | 6.2% |
| Example 43 | NG-536 | 2 μM | 9.8% |
| Example 45 | NG-550 | 10 μM | |
| Example 48 | NG-561 | 25 μM | |
| Example 49 | NG-562 | 20 μM | |
| Example 51 | NG-576 | 15 μM | 31.8% |
| Example 53 | NG-582 | 1.5 μM | 0.0% |
| Example 54 | NG-584 | 20 μM | 0.0% |
| Example 55 | NG-590 | 3 μM | 1.4% |
| Example 56 | NG-592 | 7 μM | 0.0% |
| Example 57 | NG-593 | 2 μM | |
| Example 58 | NG-594 | 7 μM | 0.0% |
| Example 59 | NG-595 | 3 μM | |
| Example 61 | NG-597 | 1 μM | |
| Example 62 | NG-598 | 2 μM | |
| Example 63 | NG-599 | 6 μM | |
| Example 64 | NG-601 | 6 μM | |
| Example 65 | NG-602 | 6 μM | |
| Example 66 | NG-605 | 10 μM | |
| Example 67 | NG-608 | 2 μM | |
| Example 68 | NG-609 | 1.5 μM | 0.0% |
| Example 69 | NG-610 | 1 μM | |
| Example 70 | NG-613 | 3 μM | 0.0% |
| Example 71 | NG-614 | 2 μM | |
| Example 73 | NG-616 | 3 μM | |
| Example 76 | NG-619 | 10 μM | 5.1% |
| Example 77 | NG-620 | 5 μM | 0.0% |
| Example 80 | NG-634 | 50 μM | 3.4% |
| Example 83 | NG-637 | 2.5 μM | 0.1% |
| Example 84 | NG-639 | 10 μM | 0.0% |
| Example 85 | NG-642 | 3 μM | 0.0% |
| Example 86 | NG-643 | 1.5 μM | 0.0% |
| Example 87 | NG-647 | 3 μM | 0.0% |
| Example 88 | NG-648 | 25 μM | 0.0% |
| Example 89 | NG-652 | 25 μM | 0.0% |
| Example 90 | NG-658 | 3 μM | |
| Example 91 | NG-662 | 50 μM | |
| Example 92 | NG-666 | 3 μM | |
| Example 93 | STS-9 | 20 μM | |
| Example 94 | STS-15 | 2 μM | 0.0% |
| Example 95 | STS-18 | 3 μM | 13.1% |
| Example 96 | STS-19 | 10 μM | |
| Example 97 | STS-25 | 20 μM | |
| Example 98 | CLF-3-205 | 12 μM | 0.0% |
| Example 99 | CLF-3-206 | 12 μM | 37.3% |
| Example 100 | CLF-3-213 | 20 μM | 0.0% |
| Example 101 | AM-50 | 2.5 μM | 23.6% |
| Example 102 | AM-1-71 | 6 μM | 21.5% |
| Example 103 | AM-1-74 | 25 μM | 33.9% |
| Example 104 | AM-1-75 | 12 μM | 0.0% |
| Example 105 | AM-1-82 | 3 μM | 12.2% |
| Example 106 | AM-1-93a | 5 μM | 21.6% |
| Example 108 | AM-1-98 | 5 μM | 8.0% |
| Example 109 | AM-2-102a | 10 μM | 0.0% |
| Example 110 | AM-2-102b | 25 μM | 0.0% |
| Example 111 | AM-2-102c | 25 μM | 0.0% |
| Example 112 | AM-1-103 | 10 μM | 1.3% |
| Example 113 | AM-1-109 | 6 μM | 24.6% |
| Example 114 | AM-2-178a | 3 μM | 7.0% |
| Example 116 | AM-2-180b | 12 μM | 0.0% |
| Example 117 | AM-3-183b | 3 μM | 7.4% |
| Example 118 | AM-3-190a | 25 μM | 0.0% |
| Example 120 | AMU-18 | 4 μM | 40.5% |
| Example 121 | AMU-19 | 12 μM | 41.2% |
| Example 122 | NP22c | 8 μM | 2.1% |
| Example 123 | NP22d | 3 μM | 10.6% |
| Example 124 | AM-62 | 17 μM | 33.7% |
| Example 125 | AM-2-137 | 25 μM | 11.1% |
| Example 126 | AM-2-139 | 5 μM | 2.2% |
| Example 127 | AM-2-179 | 5 μM | 0.0% |
| Example 129 | AM-3-183a | 6 μM | 5.1% |
| Example 130 | AM-3-187a | 3 μM | 13.9% |
| Example 133 | AM-3-188b | 25 μM | 0.0% |
| Example 135 | AM-3-210 | 3 μM | 5.8% |
| Example 136 | AM-3-213a | 20 μM | 9.8% |
| Example 139 | AM-3-239e | 15 μM | 0.0% |
| Example 143 | AM-4-254 | 20 μM | 16.8% |
| Example 147 | AMU-21 | 3 μM | 30.0% |
| Example 148 | AMU-28 | 2 μM | |
| Example 149 | AMU-29 | 3 μM | |
| Example 150 | TSch-61a | 5 μM | 6.3% |
| Example 151 | TSch-61d | 2.5 μM | 12.1% |
| Example 152 | TSch-62a | 10 μM | 74.0% |
| Example 153 | TSch-62b | 5 μM | 42.2% |
| Example 155 | LS-8 | 15 μM | |
| Example 156 | LS-9 | 15 μM | |
| Example 157 | LS-11 | 0.9 μM | |
| Example 158 | LS-12 | 15 μM | |
| Example 159 | LS-17 | 20 μM | |
| Example 160 | LS-18 | 15 μM | |
| Example 164 | OKO-06 | 25 μM | |
| Example 165 | OKO-31 | 5 μM | |
| Example 167 | LS-7 | 10 μM | |
| Example 171 | RE-10 | 5 μM | |
| Example 172 | RE-18 | 20 μM | |
| Example 173 | RE-16a | 12 μM | |
| Example 174 | RE-16b | 25 μM | |
| Example 177 | OKO-13 | 25 μM | 0.0% |
| Example 187 | BB-3-106 | 15 μM | 10.2% |

It has thus been demonstrated that the compounds of formula (I) exhibit a remarkably potent inhibitory activity against human ATGL, which renders these compounds highly advantageous for human medicinal use.

Moreover, various exemplary compounds have additionally been found to exhibit cross-species reactivity, inhibiting not only human ATGL but also murine ATGL. This property makes the corresponding compounds particularly suitable for preclinical development, as they can readily be tested in mouse models. For instance, the compound of Example 152 (Tsch-62A) exhibits a particularly advantageous cross-species activity, as illustrated in FIG. 2B.

### Example 238: Biological Experiments (II)

Methods

Ki Values [nM]

For determination of Ki values, lysates from Expi cells overexpressing human ATGL were incubated with recombinant purified ATGL co-activator CGI-58 and ATGL inhibitors at a single concentration dependent on the previously determined IC₅₀. Increasing concentrations of substrate containing radiolabeled triolein were added and liberated FA were extracted and quantified by liquid scintillation. Inhibitors were dissolved in DMSO which was used as control. Ki was determined via non-linear regression analysis. Samples were measured in triplicates.

IC₅₀ SGBS FA Release [μM]

Human differentiated SGBS adipocytes were pretreated with DMSO or ATGL inhibitors for 2 h. Subsequently, lipolysis was stimulated with 1 μM Isoproterenol for 1 h and fatty acid release was determined from media via NEFA Reagent (Wako Diagnostics). Samples were measured in triplicates.

IC₅₀ SGBS Glycerol Release [μM]

Human differentiated SGBS adipocytes were pretreated with DMSO or ATGL inhibitors for 2 h. Subsequently, lipolysis was stimulated with 1 μM Isoproterenol for 1 h and glycerol release was determined from media via Free glycerol reagent (Sigma Aldrich). Samples were measured in triplicates.

IC₅₀ hMADS FA Release [μM]

Human differentiated hMADS adipocytes were pretreated with DMSO or ATGL inhibitors for 2 h. Subsequently, lipolysis was stimulated with 1 μM Isoproterenol for 1 h and fatty acid release was determined from media via NEFA Reagent (Wako Diagnostics). Samples were measured in triplicates.

IC₅₀ hMADS Glycerol Release [μM]

Human differentiated hMADS adipocytes were pretreated with DMSO or ATGL inhibitors for 2 h. Subsequently, lipolysis was stimulated with 1 μM Isoproterenol for 1 h and glycerol release was determined from media via Free glycerol reagent (Sigma Aldrich). Samples were measured in triplicates.

IC₅₀ 3 T3 FA Release [μM]

Mouse differentiated 3T3-L1 adipocytes were pretreated with DMSO or ATGL inhibitors for 2 h. Subsequently, lipolysis was stimulated with 1 μM Isoproterenol for 1 h and fatty acid release was determined from media via NEFA Reagent (Wako Diagnostics). Samples were measured in triplicates.

IC₅₀ 3T3 Glycerol Release [μM]

Mouse differentiated 3T3-L1 adipocytes were pretreated with DMSO or ATGL inhibitors for 2 h. Subsequently, lipolysis was stimulated with 1 μM Isoproterenol for 111 and glycerol release was determined from media via Free glycerol reagent (Sigma Aldrich). Samples were measured in triplicates.

Mouse ATGL Inhibition [% Inhibition at 50 μM]

Lysates from Expi cells overexpressing ATGL from mouse (Mus musculus) were incubated with recombinant purified ATGL co-activator CGI-58 and ATGL inhibitors at 50 μM. Subsequently, substrate containing radiolabeled triolein was added and liberated FA were extracted and quantified by liquid scintillation. Inhibitors were dissolved in DMSO which was used as control. Samples were measured in triplicates.

Rat ATGL Inhibition [% Inhibition at 50 μM]

Lysates from Expi cells overexpressing ATGL from rat (Rattus norvegicus) were incubated with recombinant purified ATGL co-activator CGI-58 and ATGL inhibitors at 50 μM. Subsequently, substrate containing radiolabeled triolein was added and liberated FA were extracted and quantified by liquid scintillation. Inhibitors were dissolved in DMSO which was used as control. Samples were measured in triplicates.

Rhesus Monkey ATGL inhibition [% Inhibition at 50 μM]

Lysates from Expi cells overexpressing ATGL from rhesus monkeys (Macaca mulatta) were incubated with recombinant purified ATGL co-activator CGI-58 and ATGL inhibitors at 50 μM. Subsequently, substrate containing radiolabeled triolein was added and liberated FA were extracted and quantified by liquid scintillation. Inhibitors were dissolved in DMSO which was used as control. Samples were measured in triplicates.

Pig vWAT Inhibition [% Inhibition at 50 μM]

Lysates from visceral adipose tissue of pigs (Sus scrofa) were incubated with recombinant purified ATGL co-activator CGI-58 and ATGL inhibitors at 50 μM. Subsequently, substrate containing radiolabeled triolein was added and liberated FA were extracted and quantified by liquid scintillation. Inhibitors were dissolved in DMSO which was used as control. Samples were measured in triplicates.

Goat ATGL inhibition [% Inhibition at 50 μM]

Lysates from Expi cells overexpressing ATGL from goat (Capra hircus) were incubated with recombinant purified ATGL co-activator CGI-58 and ATGL inhibitors at 50 μM. Subsequently, substrate containing radiolabeled triolein was added and liberated FA were extracted and quantified by liquid scintillation. Inhibitors were dissolved in DMSO which was used as control. Samples were measured in triplicates.

hPNPLA9 Off-Target Inhibition [% Inhibition at 100 μM]

Expi lysates expressing hPNPLA9 were treated with 100 μM Inhibitors and incubated with 1 mM C8 monoacylglycerol substrate (containing 5 mM CHAPS, 50 mM NaPh buffer ph7.4, 0.15 M NaCl) for 40 min. Glycerol release was determined via Free glycerol reagent (Sigma Aldrich). Samples measured in triplicates,

hPNPLA6 Off-Target Inhibition [% Inhibition at 100 μM]

Expi lysates expressing hPNPLA6 were treated with 100 μM Inhibitors and incubated with 1 mM C8 monoacylglycerol substrate (containing 5 mM CHAPS, 50 mM NaPh buffer ph7.4, 0.15 M NaCl) for 40 min, Glycerol release was determined via Free glycerol reagent (Sigma Aldrich). Samples measured in triplicates.

mMGL Off-Target Inhibition [% Inhibition at 100 μM]

Lysates from E. coli expressing mMGL were treated with 100 μM Inhibitors and incubated with 1 mM rac-OG substrate for 10 min. Enzyme activity was measured using the Free Glycerol Reagent. Samples measured in triplicates.

hHSL Off-Target Inhibition [% Inhibition at 100 μM]

Expi lysates expressing hHSL were preincubated with 100 μM Inhibitors for 30 min and incubated with 1 mM pNV substrate for 30 min. Samples measured in triplicates.

Tox HepG2 (LDH) Seen from [μM]

HepG2 cells were seeded in 96 well plates and at 50% confluency treated with DMSO (0.5% final conc.) or ATGL Inhibitors for 24 h in DMEM+P/S+3% heat inactivated FCS (3 h at 62° C.). Subsequently, LDH activity of 50 μl medium was determined via the Roche LDH Kit. Samples measured in triplicates.

Tox AML-12 (LDH) seen from [μM]

AML-12 cells were seeded in 96 well plates and at 80% confluency treated with DMSO (0.5% final conc.) or ATGL Inhibitors for 24 h in DMEM+P/S+3% heat inactivated FCS (3 h at 62° C.). Subsequently, medium was centrifuged at 300 g for 3 min, and LDH activity of 50 μl supernatant was determined via the Roche LDH Kit. Samples measured in triplicates.

Tox PBMC (LDH) Seen from [μM]

Human primary peripheral blood mononuclear cell from MUG were seeded in 96 well plates treated with DMSO (0.25% final conc.) or ATGL Inhibitors for 24 h in RPMI+P/S+5% heat inactivated FCS (3 h at 62° C.). Subsequently, LDH activity of 10 μl supernatant was determined via the Roche LDH Kit. Samples measured in triplicates.

Stability in Human Serum [% Rest after 3 h Incubation]

Inhibitors were incubated in human serum for 0 or 3 h at 37° C. and subsequently extracted with the MTBE method and analyzed via HPLC MS. Samples measured in triplicates.

The results obtained in these experiments are summarized in the following tables as well as FIGS. 3 to 11:

| | | Inhibition of | | |
|---|---|---|---|---|
| | | human ATGL | % Inhibition of murine ATGL | |
| Compound | | IC₅₀ [μM] | at 50 μM | at 25 μM |
| Example 202 | MC-18 | 3 μM | | |
| Example 203 | MC-19 | 10 μM | | |
| Example 204 | MC-20 | 1 μM | | |
| Example 205 | MC-23 | 3 μM | | |
| Example 206 | MC-24 | 5 μM | | |
| Example 208 | LS-20/26 | 10 μM | 59% | |
| Example 209 | LS-27 | 10 μM | 76% | |
| Example 210 | LS-30 | <3 μM | 60% | 47% |
| Example 211 | LS-31 | <3 μM | 82% | 73% |
| Example 223 | LS-39 | 12 μM | 71% | |
| Example 224 | LS-40 | 12 μM | 68% | |
| Example 227 | RE-26b | 50 μM | 44% | |
| Example 212 | LS-32 | 7 μM | 56% | 47% |
| Example 215 | LYSU-1 1 | <3 μM | 81% | 71% |
| Example 216 | LYSU-12 | <3 μM | 81% | 70% |
| Example 225 | LYSU-15 | 50 μM | 68% | |
| Example 217 | LYSU-16 | 20 μM | 77% | |
| Example 230 | AM-3-220a | 50 μM | | |
| Example 218 | AM-5-305 | 20 μM | | |
| Example 220 | AM-5-306b | 30 μM | | |
| Example 231 | AM-5-309a | 30 μM | | |
| Example 232 | AM-5-309b | 12 μM | | |
| Example 233 | AM-5-309C | 10 μM | | |
| Example 201 | CLF-4-326 | <3 μM | 45% | |
| Example 189 | CLF-4-332 | 25 μM | 87% | |
| Example 190 | CLF-4-333 | 15 μM | 80% | |
| Example 197 | MC-07 | 30 μM | 75% | |
| Example 198 | MC-08 | 20 μM | 82% | |

| | Efficacy | | | | | | | | | | | | Off-targets | | | | | | | |
|---|---|---|---|---|---|---|---|---|---|---|---|---|---|---|---|---|---|---|---|---|
| | | | | | | | | mouse | rat | rhesus | pig | goat | hPNPLA9 | hPNPLA6 | mMGL | hHSL | Toxicity | | | Stability |
| | | | IC50 | | | IC50 | IC50 | ATGL | ATGL | monkey | vWAT | ATGL | off-target | off-target | off-target | off-target | Tox | Tox | Tox | Stability |
| | | IC50 | SGBS | IC50 | IC50 | 3T3 | 3T3 | inhibi - | inhibi - | ATGL | inhibi - | inhibi - | inhibi - | inhibi - | inhibi - | inhibi - | HepG2 | AML-12 | PBMC | in human |
| | | SGBS | glyc- | hMADS | hMADS | FA | glyc- | tion [% | tion [% | inhibition | tion [% | tion [% | tion [% | tion [% | tion [% | tion [% | (LDH) | (LDH) | (LDH) | serum |
| | Ki | FA | erol | FA | glycerol | re- | erol | inhibi- | inhibi- | [% | inhibi- | inhibi- | inhibi- | inhibi- | inhibi- | inhibi- | seen | seen | seen | [% rest |
| | values | release | release | release | release | lease | release | tion at | tion at | inhibition | tion at | tion at | tion at | tion at | tion at | tion at | from | from | from | after 3 h |
| | [nM] | [μM) | [μM) | [μM) | [μM) | [μM) | [μM) | 50 μM] | 50 μM] | at 50 μM] | 50 μM | 50 μM | 100 μM] | 100 μM] | 100 μM] | 100 μM] | [μM] | [μM] | [μM] | incubation] |
| Example 105 | | 3 | 3 | | | | | | | | | | | | | | | | | |
| (AM-1-82) | | | | | | | | | | | | | | | | | | | | |
| Example 101 | 2500 | 3 | 3 | | | | | 30 | | | | | | | | | — | | — | 25 |
| (AM-50) | | | | | | | | | | | | | | | | | | | | |
| Example 120 | 1200 | | | | | | | 40 | | | | | | | | | — | | | |
| (AMU-18) | | | | | | | | | | | | | | | | | | | — | 5 |
| Example 148 | | 2 | 3 | | | | | 30 | | | | | | | | | | | | |
| (AMU-28) | | | | | | | | | | | | | | | | | | | | |
| Example 201 | | | | | | | | 0 | 0 | 95 | 0 | 0 | 0 | 0 | 0 | 0 | — | 200 | | 100 |
| (CLF-4-326) | | | | | | | | | | | | | | | | | | | | |
| Example 189 | | | | | | | | | 10 | 70 | 15 | 50 | 0 | 0 | 0 | 0 | | 200 | | 25 |
| (CLF-4-332) | | | | | | | | | | | | | | | | | | | | |
| Example 190 | | 12 | 25 | | | 6 | 25 | | 40 | 70 | 0 | 25 | 0 | 0 | 0 | 0 | — | 200 | | 0 |
| (CLF-4-333) | | | | | | | | | | | | | | | | | | | | |
| Example 157 | 210 | | | 2.5 | 5 | | | | | | | | | | | | | | — | |
| (LS-11) | | | | | | | | | | | | | | | | | | | | |
| Example 209 | | | | | | | | | 40 | 70 | 0 | 25 | 0 | 0 | 0 | 40 | 200 | 100 | | 75 |
| (LS-27) | | | | | | | | | | | | | | | | | | | | |
| Example 210 | | 0.6 | 1 | | | | | | 15 | 90 | 5 | 20 | 0 | 0 | 0 | 0 | — | 200 | | 100 |
| (LS-30) | | | | | | | | | | | | | | | | | | | | |
| Example 211 | | 0.9 | 0.9 | | | 4 | 20 | 60 | 50 | 100 | 5 | 30 | 0 | 0 | 0 | 40 | — | 100 | | 90 |
| (LS-31) | | | | | | | | | | | | | | | | | | | | |
| Example 212 | | | | | | | | | 40 | 70 | 5 | 20 | 0 | 0 | 0 | 0 | — | 200 | | 50 |
| (LS-32) | | | | | | | | | | | | | | | | | | | | |
| Example 223 | | | | | | | | | 40 | 65 | 15 | 40 | 0 | 0 | 0 | 40 | — | — | | 80 |
| (LS-39) | | | | | | | | | | | | | | | | | | | | |
| Example 224 | | | | | | | | | 40 | 70 | 15 | 40 | 0 | 0 | 70 | 95 | — | — | | |
| (LS-40) | | | | | | | | | | | | | | | | | | | | |
| Example 215 | | 3 | 4 | | | 6 | 15 | 60 | 50 | 70 | 15 | 50 | 0 | 0 | 0 | 30 | — | — | | 80 |
| (LYSU-11) | | | | | | | | | | | | | | | | | | | | |
| Example 216 | | 0.8 | | | | 10 | 25 | | 50 | 70 | 15 | 50 | 0 | 0 | 0 | 30 | — | 50 | | 60 |
| (LYSU-12) | | | 1 | | | | | | | | | | | | | | | | | |
| Example 6 | | 2 | | | | | | | | | | | 0 | | | | | | | |
| (NG-399) | | | | | | | | | | | | | | | | | | | | |
| Example 35 | 500 | 1 | 2 | | | | | | | | | | 0 | | 0 | 0 | — | | — | 75 |
| (NG-497) | | | | | | | | | | | | | | | | | | | | |
| Example 41 | 2500 | | | | | | | | | | | | | | | | — | | — | 75 |
| (NG-531) | | | | | | | | | | | | | | | | | | | | |
| Example 43 | 470 | | | 3 | | | | | | | | | | | | | — | | | 80 |
| (NG-536) | | | | | | | | | | | | | | | | | | | | |
| Example 53 | 550 | | | | | | | | | | | | | | | | — | | | 80 |
| (NG 582) | | | | | 80 | | | | | | | | | | | | | | | |
| Example 61 | 420 | | | 2 | | | | | | | | | | | | | 50 | | — | 75 |
| (NG-597) | | | | | | | | | | | | | | | | | | | | |
| Example 68 | 270 | | | 1 | | | | | | | | | | | | | — | | | 80 |
| (NG 609) | | | | | | | | | | | | | | | | | | | | |
| Example 69 | 830 | | | | | | | | | | | | | | | | — | | — | 80 |
| (NG 610) | | | | | 80 | | | | | | | | | | | | | | | |
| Example 86 | 470 | | | 1 | | | | | | | | | | | | | — | | | 80 |
| (NG-643) | | | | | | | | | | | | | | | | | | | | |
| Example 123 | | | | | | | | 20 | | | | | | | | | | | | |
| (NP 22D) | | | | | | | | | | | | | | | | | | | | |
| Example 152 | 8200 | | | 12 | 10 | | | | 10 | 10 | 0 | 0 | 0 | 0 | | 25 | 200 | — | — | 80 |
| (TSch-62A) | | | | | | | | | | | | | | | | | | | | |

These results further confirm that the compounds of formula (I) are highly potent inhibitors of human ATGL, and that they exhibit advantageous properties in terms of efficacy, off-target effects, toxicity and stability.

1. A compound of the following formula (I) 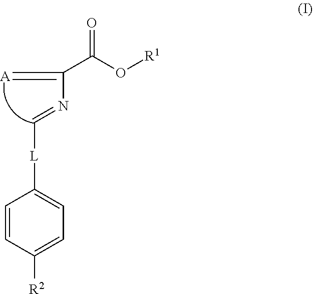
or a pharmaceutically acceptable salt or solvate thereof,
for use in treating or preventing a disease or disorder selected from a lipid metabolism disorder, obesity, non-alcoholic fatty liver disease, type 2 diabetes, insulin resistance, glucose intolerance, hypertriglyceridemia, metabolic syndrome, cardiac and skeletal muscle steatosis, congenital generalized lipodystrophy, familial partial lipodystrophy, acquired lipodystrophy syndrome, atherosclerosis, and heart failure;
wherein:
A is —CH═C(R^{A1})—CH═ or —S—C(R^{A2})═;
L is selected from a covalent bond, C₁₋₅ alkylene, C₂₋₅ alkenylene, and C₂₋₅ alkynylene, wherein one —CH₂— unit comprised in said C₁₋₅ alkylene, said C₂₋₅ alkenylene or said C₂₋₅ alkynylene is optionally replaced by —O—;
R¹ is selected from C₁₋₁₀ alkyl, C₂₋₁₀ alkenyl, C₂₋₁₀ alkynyl, carbocyclyl, and heterocyclyl, wherein said alkyl, said alkenyl and said alkynyl are each optionally substituted with one or more groups R^{Alk}, and wherein said carbocyclyl and said heterocyclyl are each optionally substituted with one or more groups R^{Cyc};
R² is selected from hydrogen, C₁₋₁₀ alkyl, C₂₋₁₀ alkenyl, C₂₋₁₀ alkynyl, —(C₀₋₄ alkylene)-OH, —(C₀₋₄ alkylene)-O(C₁₋₁₀ alkyl), —(C₀₋₄ alkylene)-O(C₁₋₁₀ alkylene)-OH, —(C₀₋₄ alkylene)-O(C₁₋₁₀ alkylene)-O(C₁₋₅ alkyl), —(C₀₋₄ alkylene)-O(C₁₋₅ alkylene)-O(C₁₋₅ alkylene)-OH, —(C₀₋₄ alkylene)-O(C₁₋₅ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(C₁₋₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—CO—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}-R^{X}, wherein said C₁₋₁₀ alkyl, said C₂₋₁₀ alkenyl, said C₂₋₁₀ alkynyl, each alkyl moiety in any of the aforementioned groups, and each alkylene moiety in any of the aforementioned groups are each optionally substituted with one or more groups R^{Alk}, and wherein the carbocyclyl moiety in said -L^{X}-carbocyclyl and the heterocyclyl moiety in said -L^{X}-heterocyclyl are each optionally substituted with one or more groups R^{Cyc};
R^{A1} and R^{A2} are each independently selected from hydrogen, C₁₋₅ alkyl, C₂₋₅ alkenyl, C₂₋₅ alkynyl, —(C₀₋₄ alkylene)-OH, —(C₀₋₄ alkylene)-O(C₁₋₅ alkyl), —(C₀₋₄ alkylene)-O(C₁₋₅ alkylene)-OH, —(C₀₋₄ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(C₁₋₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—CO—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups R^{Cyc};
each R^{Alk} is independently selected from —OH, —O(C₁₋₅ alkyl), —O(C₁₋₅ alkylene)-O, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁, haloalkyl, —O(C₁₋₅ haloalkyl), —CN, —CHO, —CO(C₁₋₅ alkyl), —COOH, —CO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups R^{Cyc};
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—CO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —CO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl);
each L^{X} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{X} is independently selected from hydrogen, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), carbocyclyl, and heterocyclyl, wherein said carbocyclyl and said heterocyclyl are each optionally substituted with one or more groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl).

2. The compound for use according to claim 1, wherein A is —CH═C(R^{A1})—C═, and said compound has the following structure:

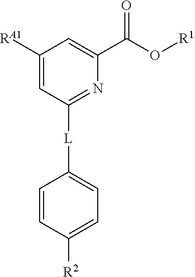

3. The compound for use according to claim 1, wherein A is —S—C(R^{A2})═, and said compound has the following structure:

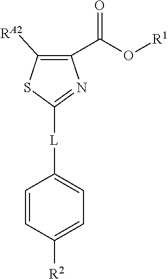

4. The compound for use according to any one of claims 1 to 3, wherein L is a covalent bond.

5. The compound for use according to any one of claims 1 to 4, wherein R¹ is selected from C₁₋₆ alkyl, C₂₋₆ alkenyl, C₂₋₆ alkynyl, cycloalkyl, and heterocycloalkyl.

6. The compound for use according to any one of claims 1 to 5, wherein R¹ is selected from ethyl, isopropyl, —CH₂—CH═CH₂, —CH₂—C(CH₃)═CH₂, —CH(CH₃)—C≡CH, and cyclopropyl.

7. The compound for use according to any one of claims 1 to 6, wherein R¹ is ethyl or isopropyl.

8. The compound for use according to any one of claims 1 to 7, wherein R² is selected from C₁₋₁₀ alkyl, —O(C₁₋₁₀ alkyl), —(C₁₋₄ alkylene)-O(C₁₋₁₀ alkyl), —O(C₁₋₁₀ alkylene)-O(C₁₋₅ alkyl), —(C₁₋₄ alkylene)-O(C₁₋₁₀ alkylene)-O(C₁₋₅ alkyl), —O(C₁₋₅ alkylene)-O(C₁₋₅ alkylene)-O(C₁₋₅ alkyl), —(C₁₋₄ alkylene)-O(C₁₋₅ alkylene)-O(C₁₋₅ alkylene)-O(C₁₋₅ alkyl), —O(C₂₋₄ alkenyl), —S(C₁₋₅ alkyl), —COO—(C₁₋₅ alkyl), —CO—N(C₁₋₅ alkyl)(C₁₋₅ alkyl), —CO—N(C₁₋₅ alkyl)-O—(C₁₋₅ alkyl), —SO₂—(C₁₋₅ alkyl), halogen, C₁₋₅ haloalkyl, —O—(C₁₋₅ haloalkyl), -L^{X}-aryl, -L^{X}-cycloalkyl, -L^{X}-heteroaryl, and -L^{X}-heterocycloalkyl, wherein the aryl moiety in said -L^{X}-aryl, the cycloalkyl moiety in said -L^{X}-cycloalkyl, the heteroaryl moiety in said -L^{X}-heteroaryl, and the heterocycloalkyl moiety in said -L^{X}-heterocycloalkyl are each optionally substituted with one or more groups R^{Cyc}.

9. The compound for use according to any one of claims 1 to 8, wherein R² is selected from —CH₂CH₃, —(CH₂)₂CH₃, —(CH₂)₃CH₃, —(CH₂)₄CH₃, —(CH₂)₅CH₃, —(CH₂)₆CH₃, —O—CH₂CH₃, —O—(CH₂)₂CH₃, —O—(CH₂)₃CH₃, —O—(CH₂)₄CH₃, —O—(CH₂)₅CH₃, —O—(CH₂)₆CH₃, —CH₂—O—CH₃, and —CH(—CH₃)—O—CH₃, preferably wherein R² is —O—CH₂CH₃.

10. The compound for use according to any one of claims 1 to 9, wherein R^{A1} and R^{A2} are each independently selected from hydrogen, —CH₃, —OCH₃, —CO—CH₃, and —I.

11. The compound for use according to any one of claims 1 to 10, wherein R^{A1} and R^{A2} are each hydrogen.

12. The compound for use according to claim 1, wherein said compound is any one of the following compounds, or a pharmaceutically acceptable salt or solvate thereof:

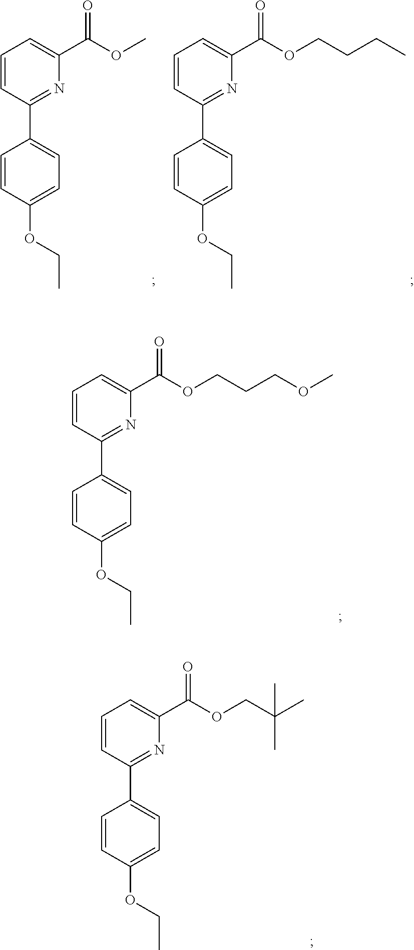

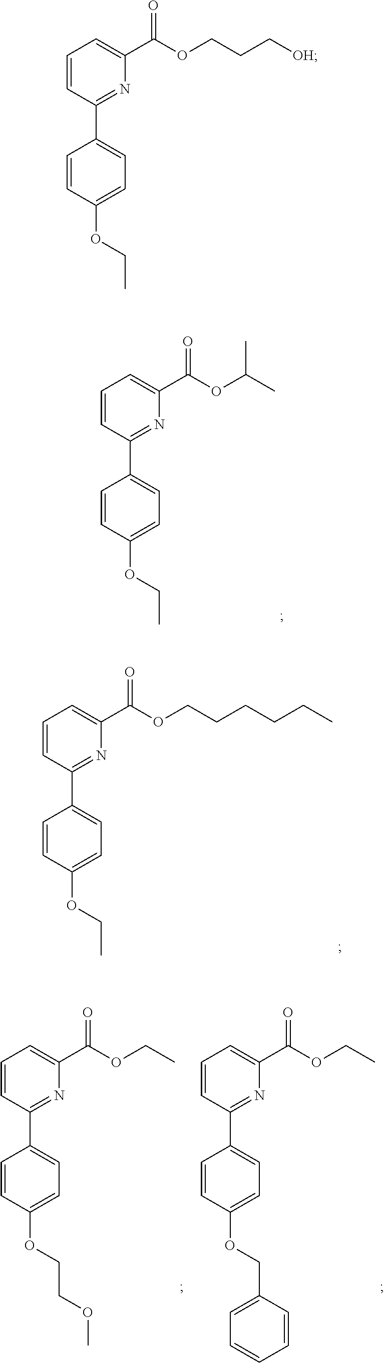

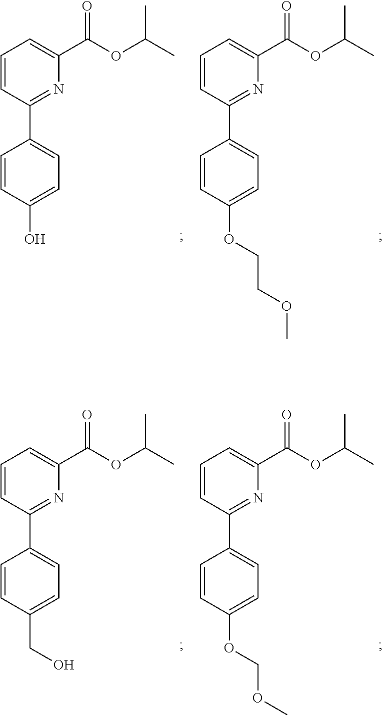

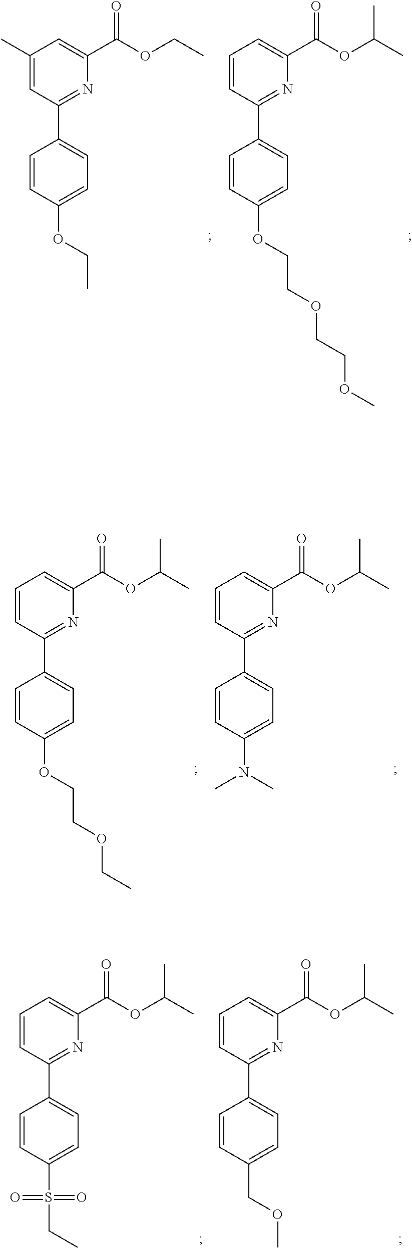

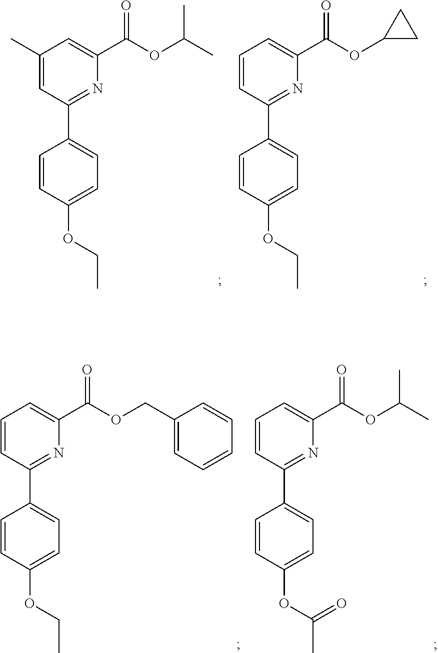

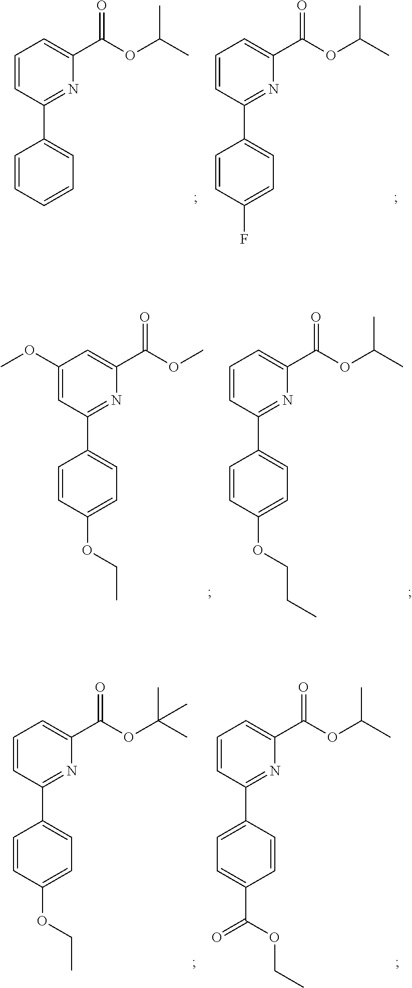

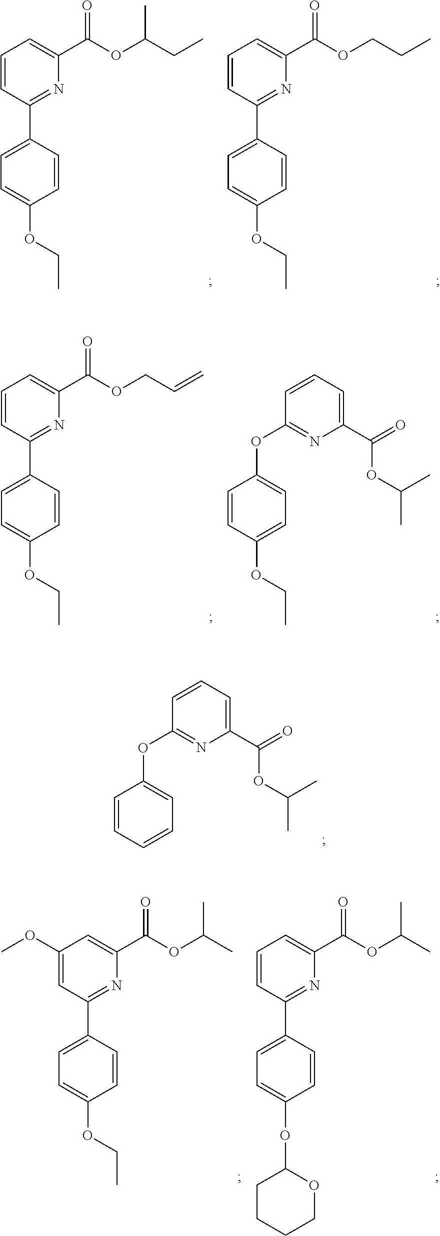

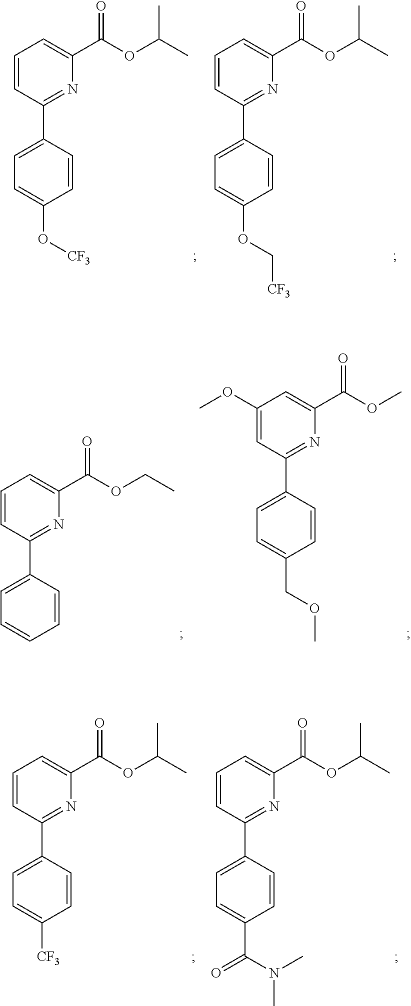

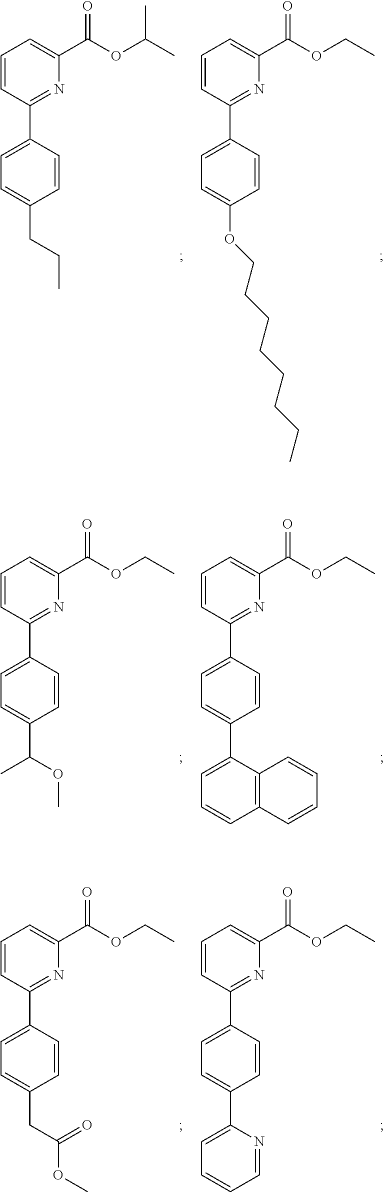

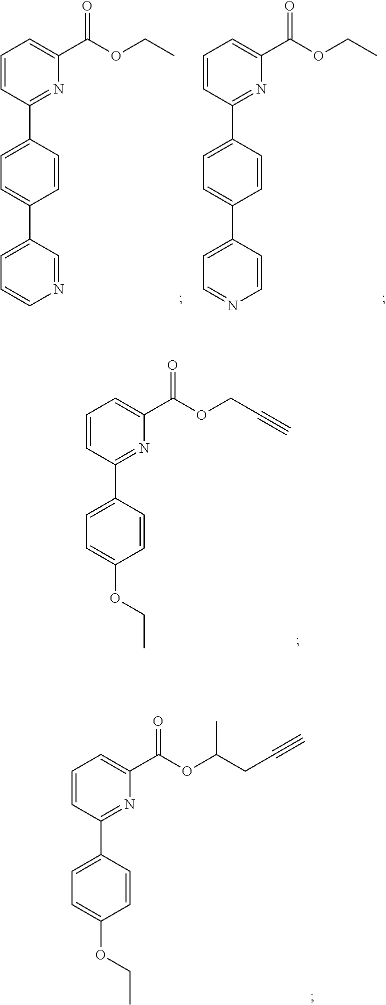

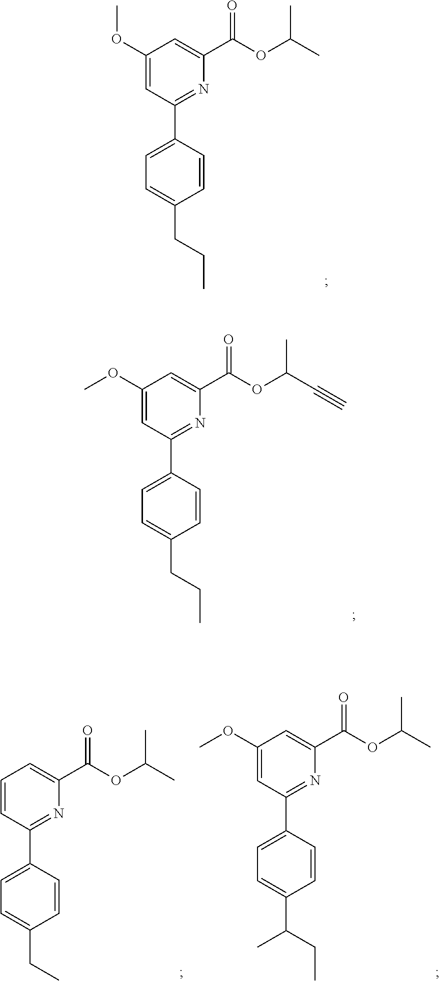

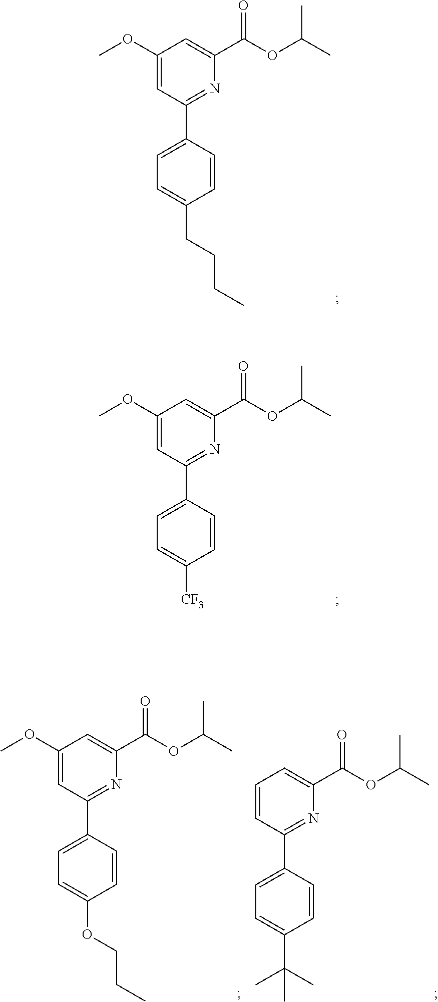

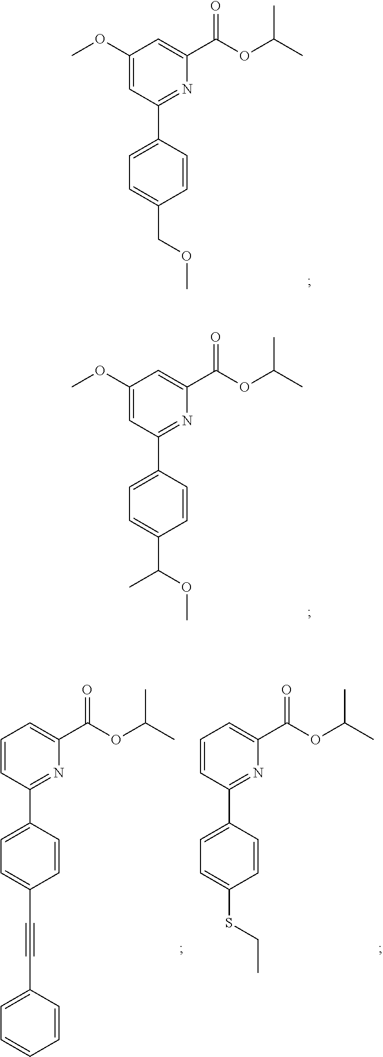

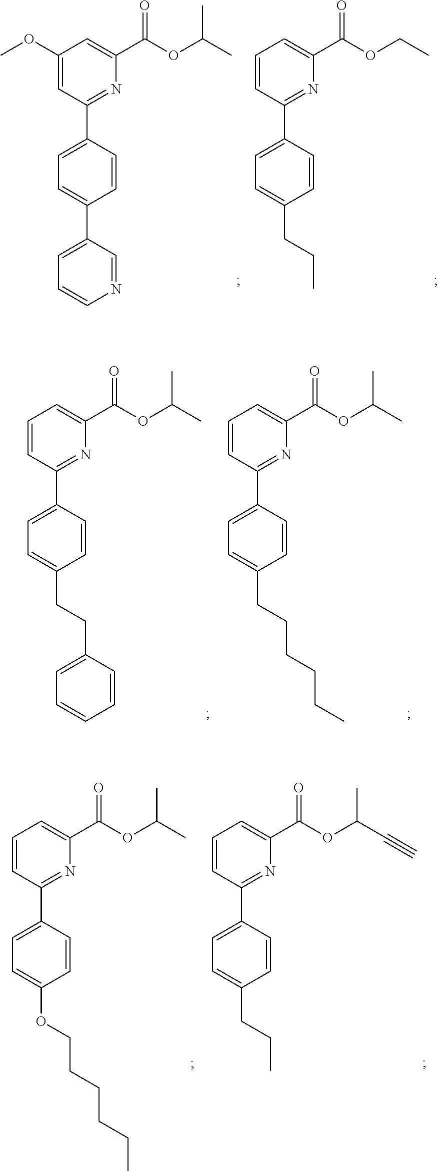

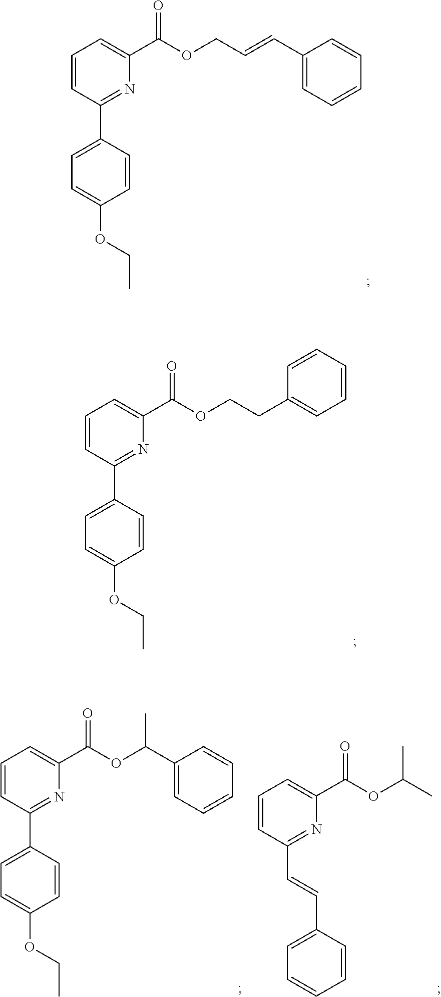

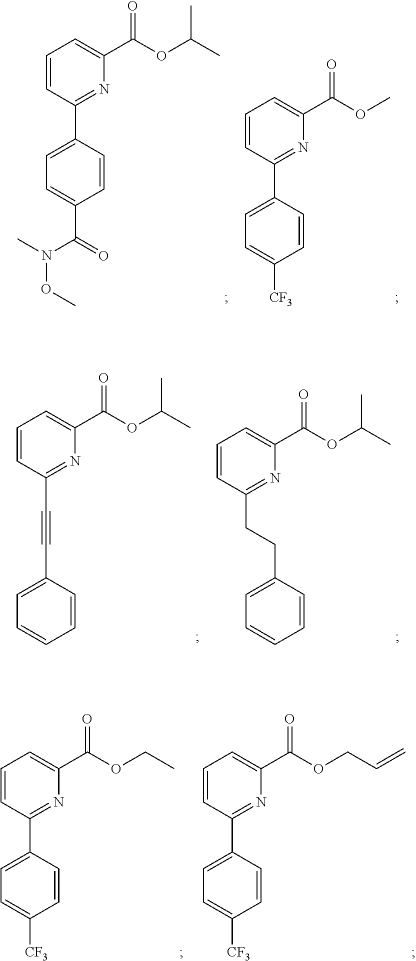

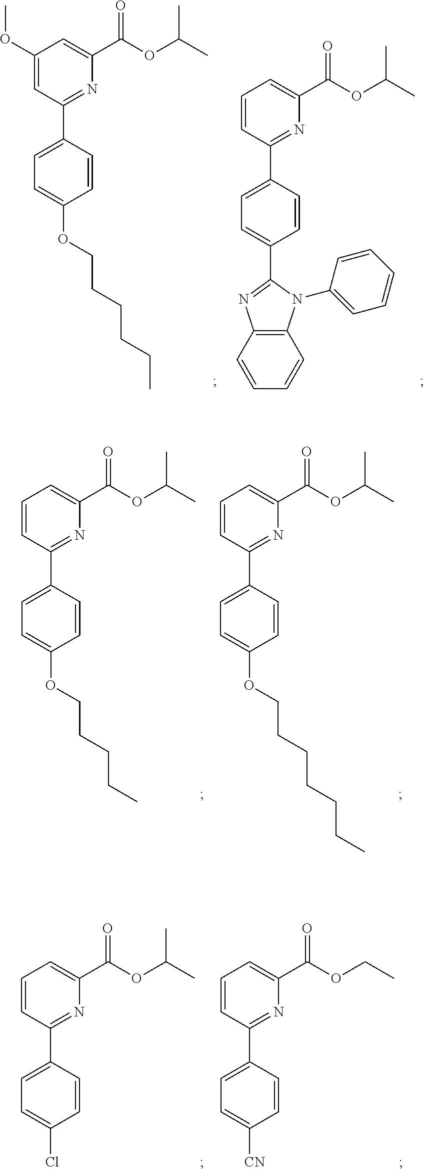

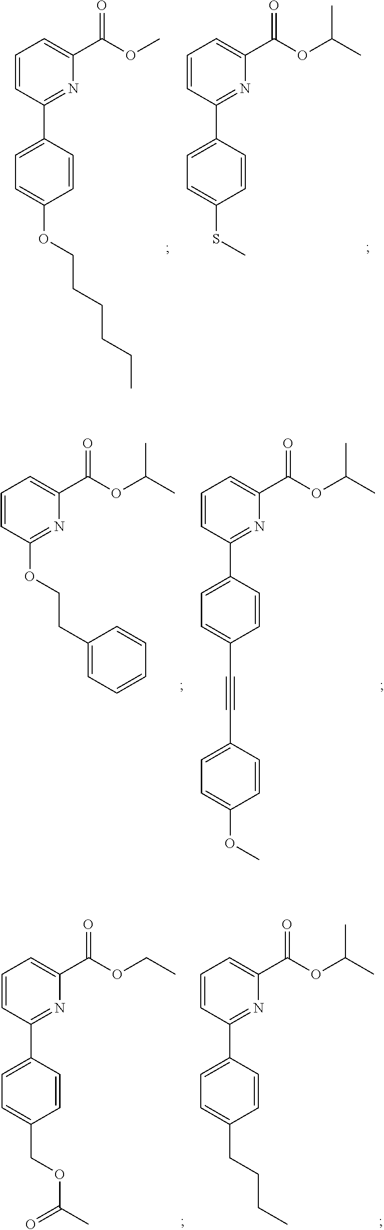

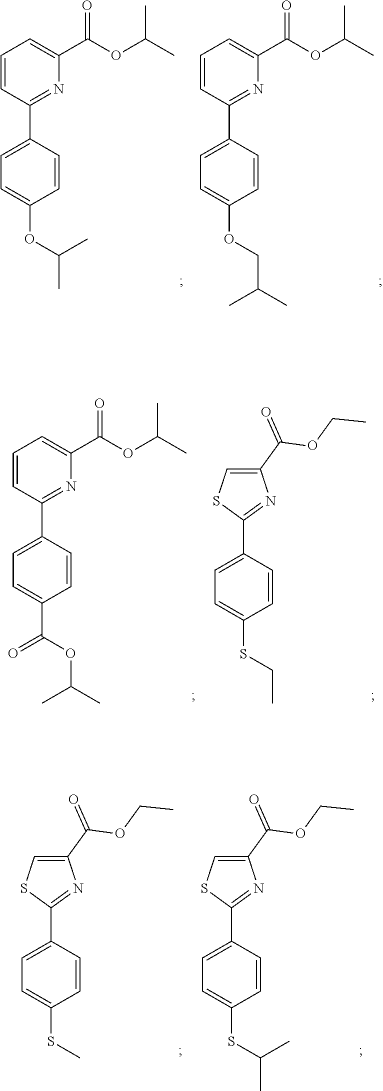

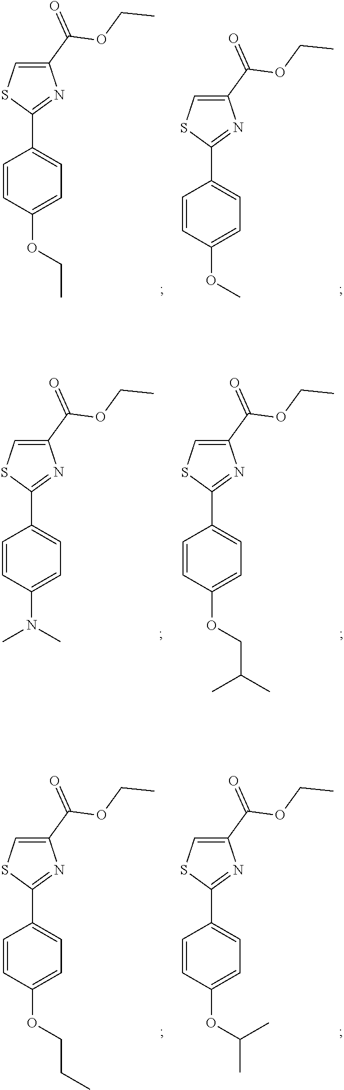 [SMILES: CCOC(=O)c1csc(-c2ccc(OCC)cc2)n1 ; CCOC(=O)c1csc(-c2ccc(OC)cc2)n1 ; CCOC(=O)c1csc(-c2ccc(N(C)C)cc2)n1 ; CCOC(=O)c1csc(-c2ccc(OCC(C)C)cc2)n1 ; CCCOc1ccc(-c2nc(C(=O)OCC)cs2)cc1 ; CCOC(=O)c1csc(-c2ccc(OC(C)C)cc2)n1]

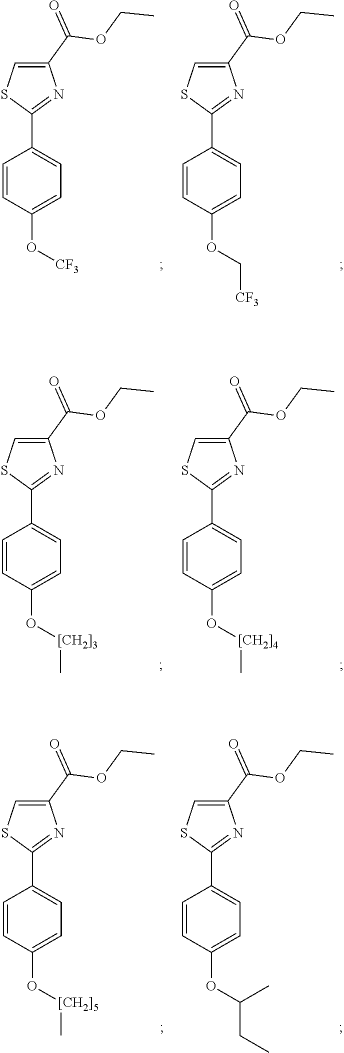

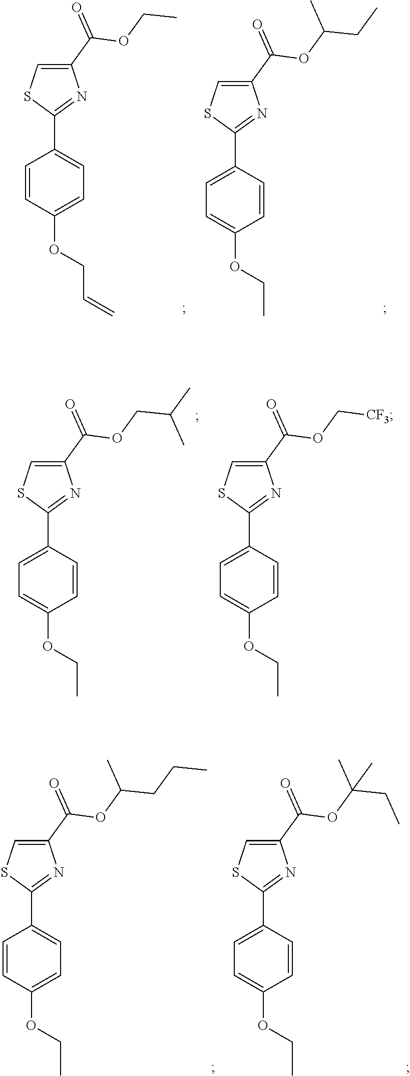 [SMILES: C=CCOc1ccc(-c2nc(C(=O)OCC)cs2)cc1 ; CCOc1ccc(-c2nc(C(=O)OC(C)CC)cs2)cc1 ; CCOc1ccc(-c2nc(C(=O)OCC(C)C)cs2)cc1 ; CCOc1ccc(-c2nc(C(=O)OCC(F)(F)F)cs2)cc1 ; CCCC(C)OC(=O)c1csc(-c2ccc(OCC)cc2)n1 ; CCOc1ccc(-c2nc(C(=O)OC(C)(C)CC)cs2)cc1]

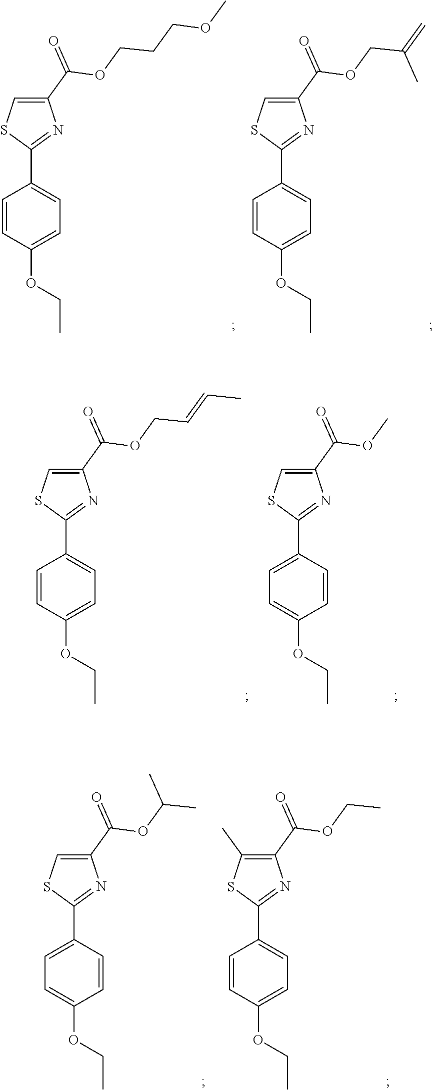

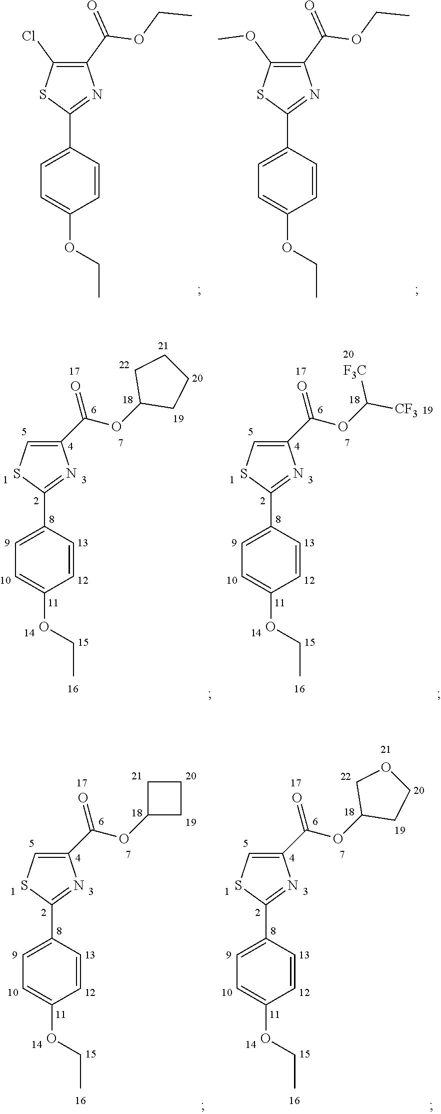

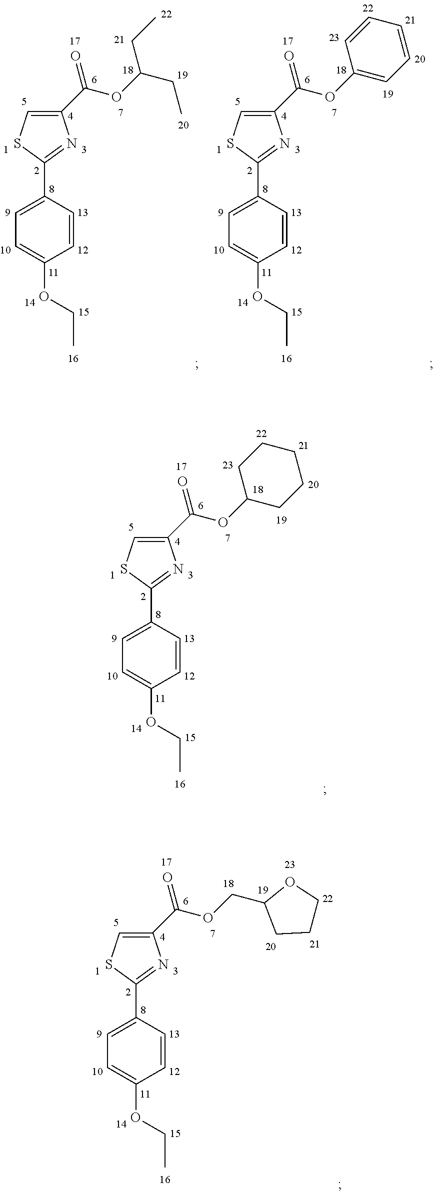 [SMILES: CCOc1ccc(-c2nc(C(=O)OC(CC)CC)cs2)cc1 ; CCOc1ccc(-c2nc(C(=O)Oc3ccccc3)cs2)cc1 ; CCOc1ccc(-c2nc(C(=O)OC3CCCCC3)cs2)cc1 ; CCOc1ccc(-c2nc(C(=O)OCC3CCCO3)cs2)cc1]

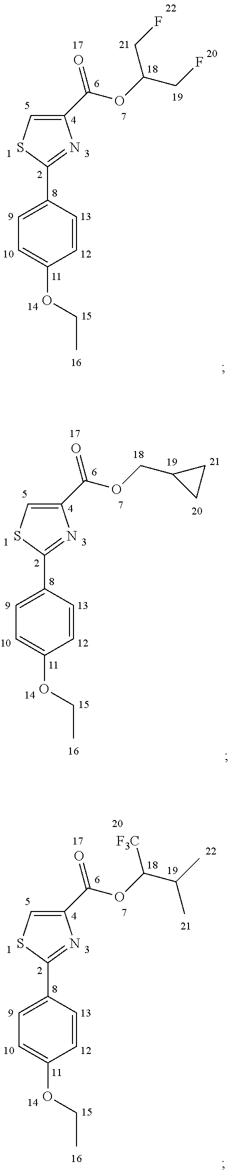 [SMILES: CCOc1ccc(-c2nc(C(=O)OC(CF)CF)cs2)cc1 ; CCOc1ccc(-c2nc(C(=O)OCC3CC3)cs2)cc1 ; CCOc1ccc(-c2nc(C(=O)OC(C(C)C)C(F)(F)F)cs2)cc1]

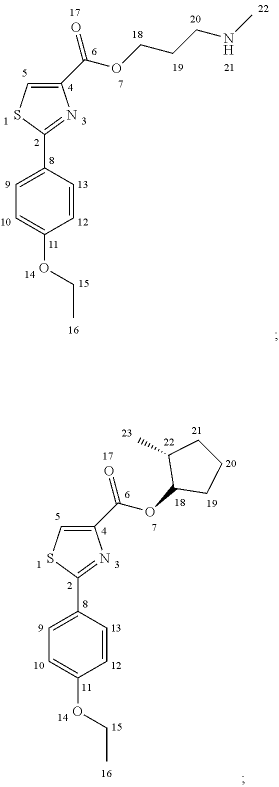

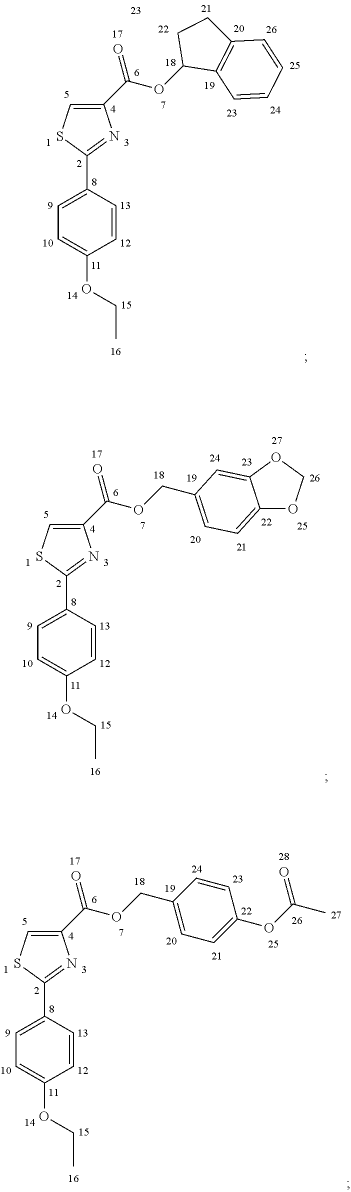 [SMILES: CCOc1ccc(-c2nc(C(=O)OC3CCc4ccccc43)cs2)cc1 ; CCOc1ccc(-c2nc(C(=O)OCc3ccc4c(c3)OCO4)cs2)cc1 ; CCOc1ccc(-c2nc(C(=O)OCc3ccc(OC(C)=O)cc3)cs2)cc1]

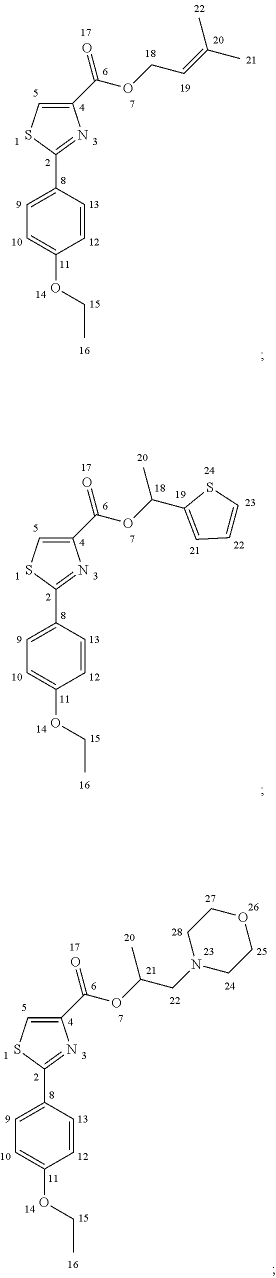 [SMILES: CCOc1ccc(-c2nc(C(=O)OCC=C(C)C)cs2)cc1 ; CCOc1ccc(-c2nc(C(=O)OC(C)c3cccs3)cs2)cc1 ; CCOc1ccc(-c2nc(C(=O)OC(C)CN3CCOCC3)cs2)cc1]

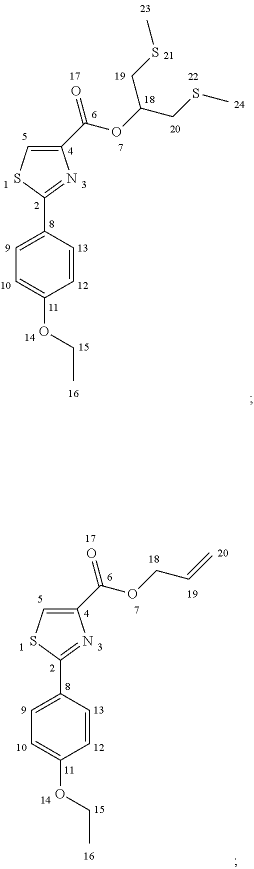 [SMILES: CCOc1ccc(-c2nc(C(=O)OC(CSC)CSC)cs2)cc1 ; C=CCOC(=O)c1csc(-c2ccc(OCC)cc2)n1]

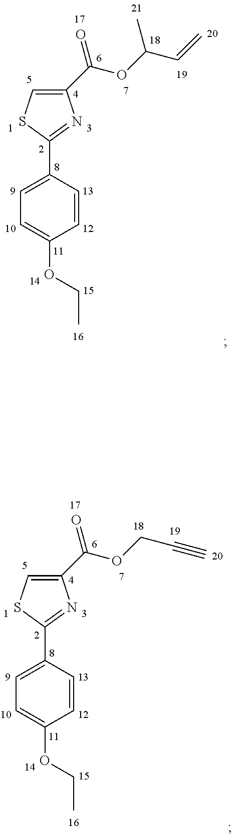 [SMILES: C=CC(C)OC(=O)c1csc(-c2ccc(OCC)cc2)n1 ; C#CCOC(=O)c1csc(-c2ccc(OCC)cc2)n1]

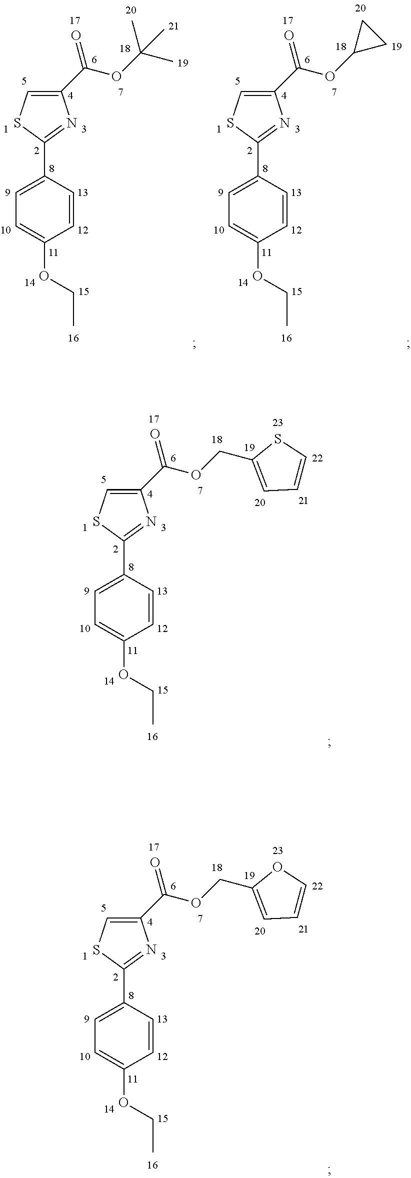 [SMILES: CCOc1ccc(-c2nc(C(=O)OC(C)(C)C)cs2)cc1 ; CCOc1ccc(-c2nc(C(=O)OC3CC3)cs2)cc1 ; CCOc1ccc(-c2nc(C(=O)OCc3cccs3)cs2)cc1 ; CCOc1ccc(-c2nc(C(=O)OCc3ccco3)cs2)cc1]

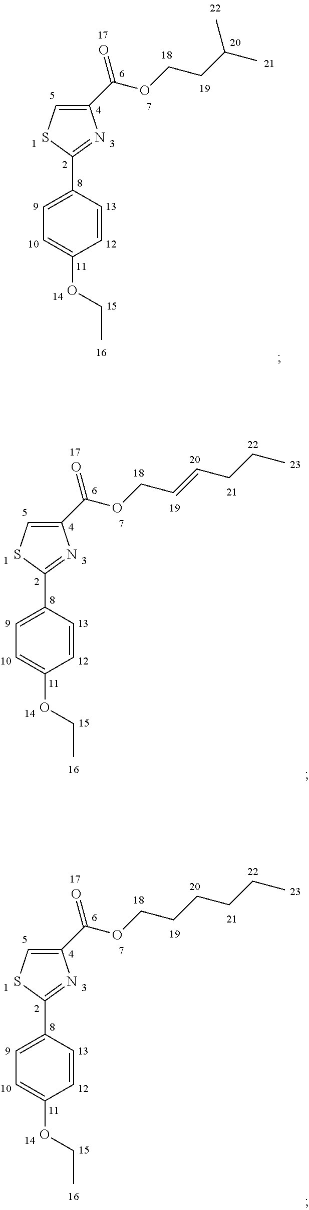 [SMILES: CCOc1ccc(-c2nc(C(=O)OCCC(C)C)cs2)cc1 ; CCC/C=C/COC(=O)c1csc(-c2ccc(OCC)cc2)n1 ; CCCCCCOC(=O)c1csc(-c2ccc(OCC)cc2)n1]

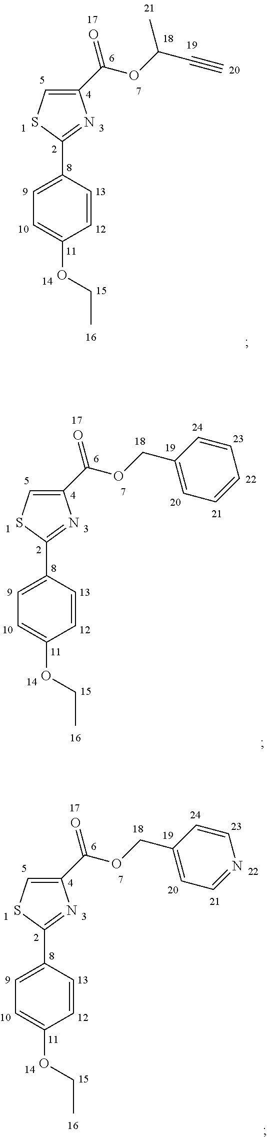 [SMILES: C#CC(C)OC(=O)c1csc(-c2ccc(OCC)cc2)n1 ; CCOc1ccc(-c2nc(C(=O)OCc3ccccc3)cs2)cc1 ; CCOc1ccc(-c2nc(C(=O)OCc3ccncc3)cs2)cc1]

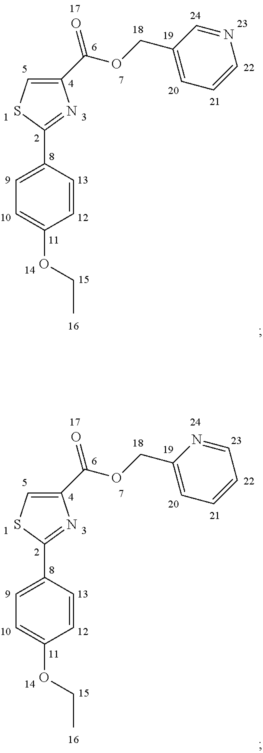 [SMILES: CCOc1ccc(-c2nc(C(=O)OCc3cccnc3)cs2)cc1 ; CCOc1ccc(-c2nc(C(=O)OCc3ccccn3)cs2)cc1]

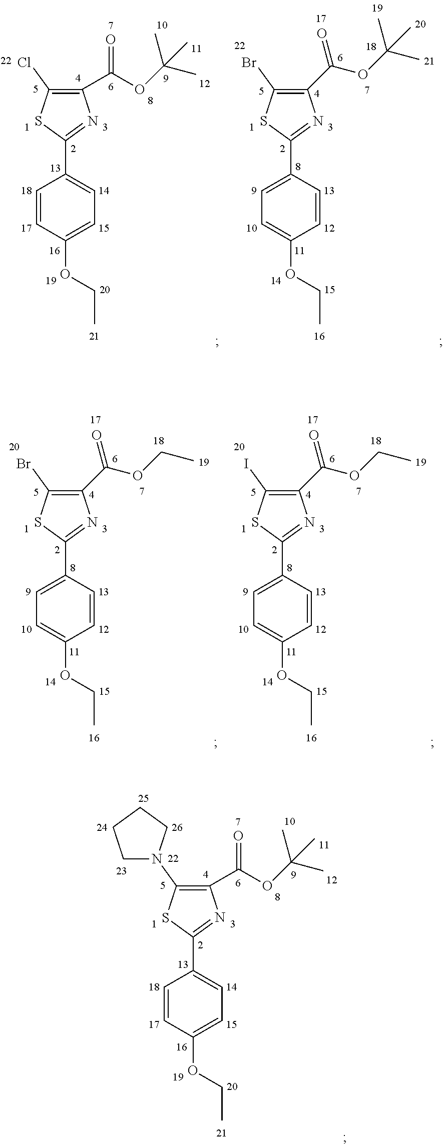 [SMILES: CCOc1ccc(-c2nc(C(=O)OC(C)(C)C)c(Cl)s2)cc1 ; CCOc1ccc(-c2nc(C(=O)OC(C)(C)C)c(Br)s2)cc1 ; CCOC(=O)c1nc(-c2ccc(OCC)cc2)sc1Br ; CCOC(=O)c1nc(-c2ccc(OCC)cc2)sc1I ; CCOc1ccc(-c2nc(C(=O)OC(C)(C)C)c(N3CCCC3)s2)cc1]

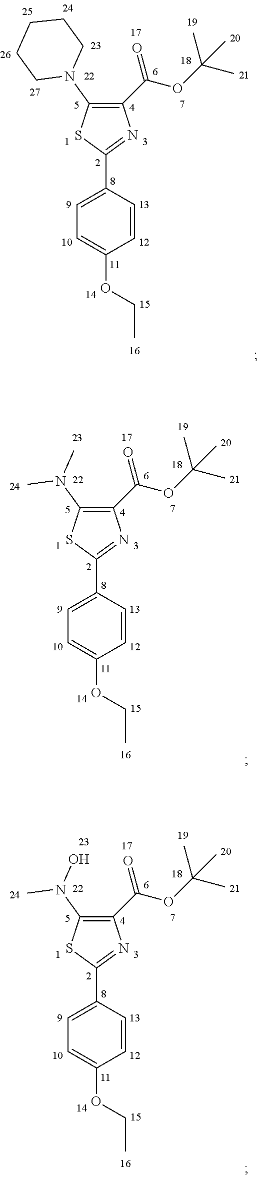

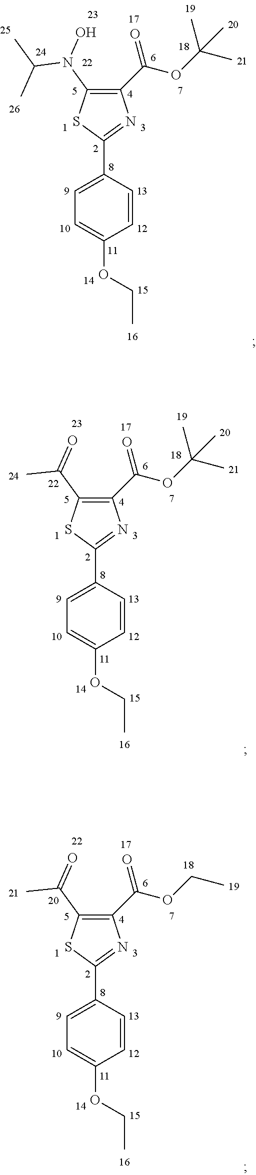

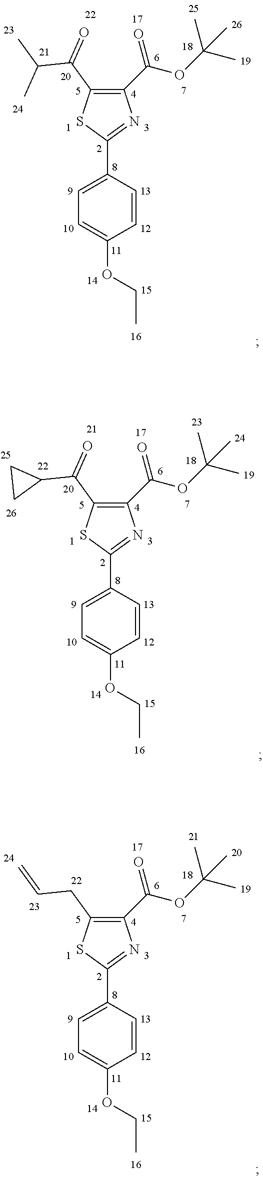

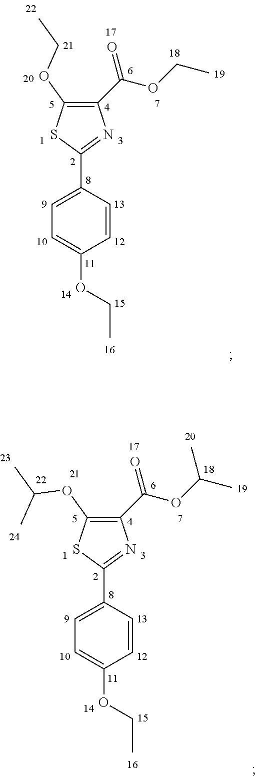

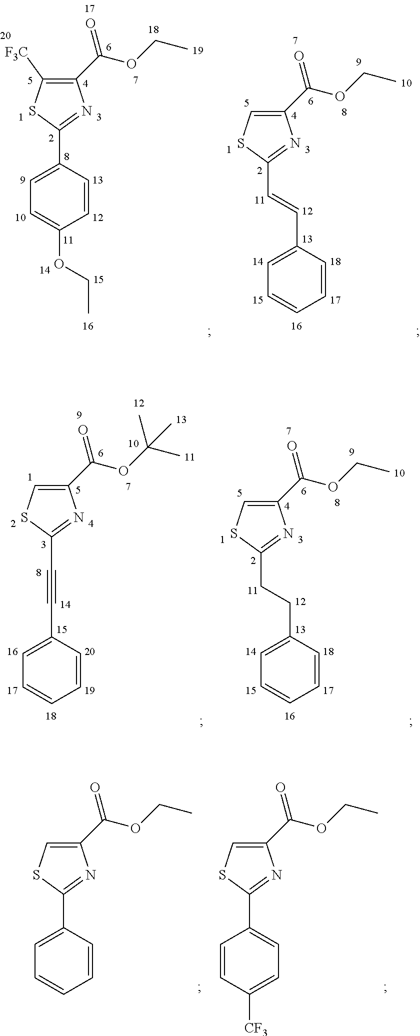 [SMILES: CCOC(=O)c1nc(-c2ccc(OCC)cc2)sc1C(F)(F)F ; CCOC(=O)c1csc(/C=C/c2ccccc2)n1 ; CC(C)(C)OC(=O)c1csc(C#Cc2ccccc2)n1 ; CCOC(=O)c1csc(CCc2ccccc2)n1 ; CCOC(=O)c1csc(-c2ccccc2)n1 ; CCOC(=O)c1csc(-c2ccc(C(F)(F)F)cc2)n1]

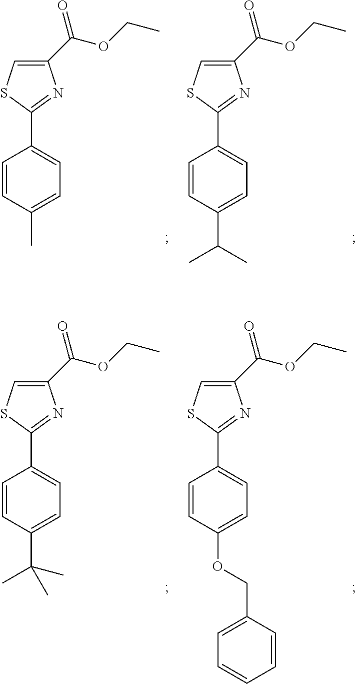 [SMILES: CCOC(=O)c1csc(-c2ccc(C)cc2)n1 ; CCOC(=O)c1csc(-c2ccc(C(C)C)cc2)n1 ; CCOC(=O)c1csc(-c2ccc(C(C)(C)C)cc2)n1 ; CCOC(=O)c1csc(-c2ccc(OCc3ccccc3)cc2)n1]

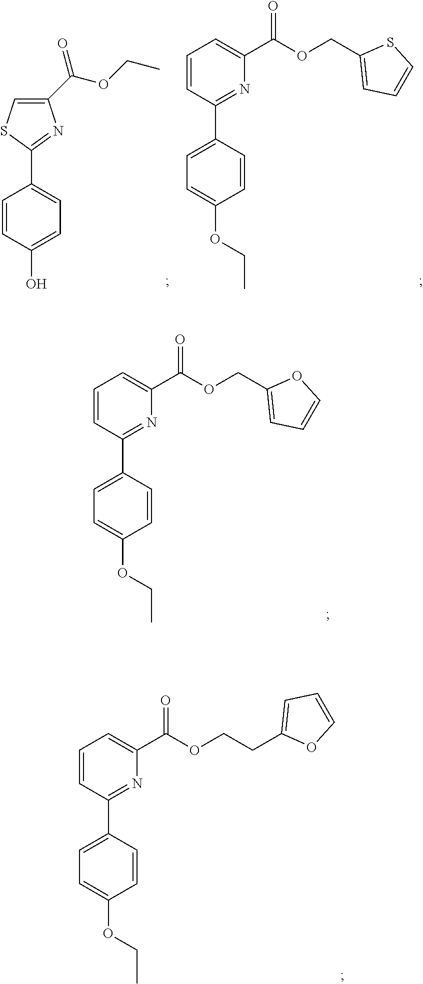

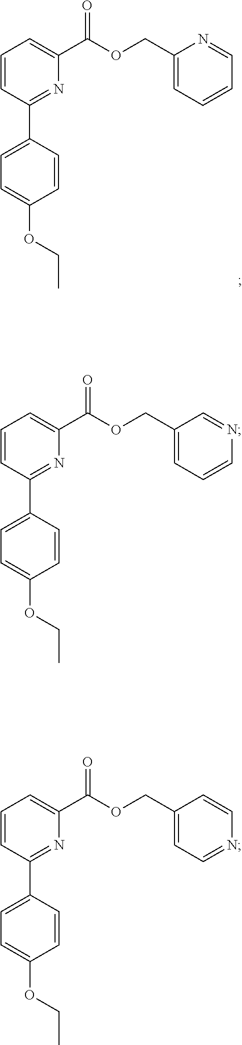 [SMILES: CCOc1ccc(-c2cccc(C(=O)OCc3ccccn3)n2)cc1 ; CCOc1ccc(-c2cccc(C(=O)OCc3cccnc3)n2)cc1 ; CCOc1ccc(-c2cccc(C(=O)OCc3ccncc3)n2)cc1]

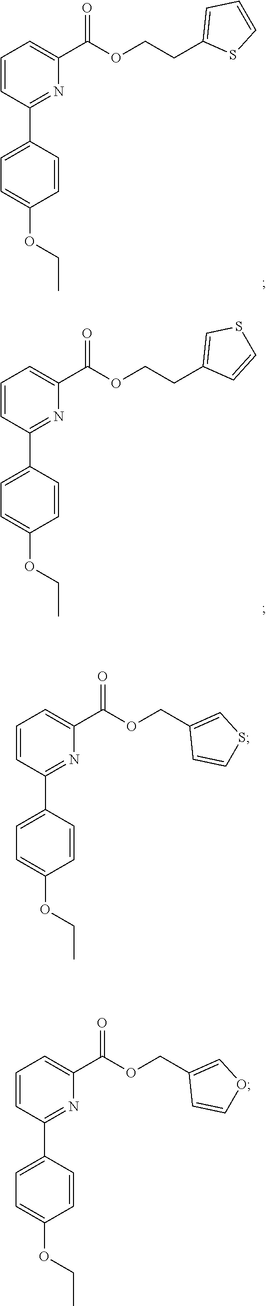 [SMILES: CCOc1ccc(-c2cccc(C(=O)OCCc3cccs3)n2)cc1 ; CCOc1ccc(-c2cccc(C(=O)OCCc3ccsc3)n2)cc1 ; CCOc1ccc(-c2cccc(C(=O)OCc3ccsc3)n2)cc1 ; CCOc1ccc(-c2cccc(C(=O)OCc3ccoc3)n2)cc1]

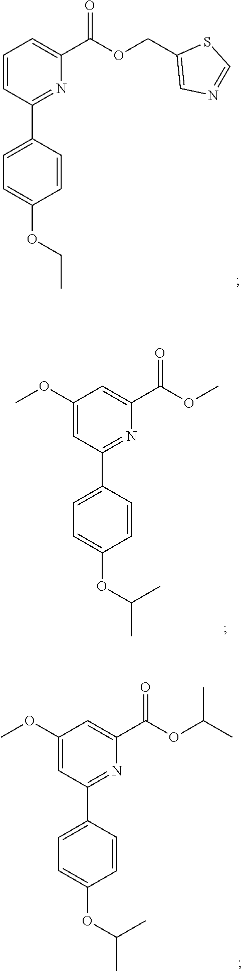

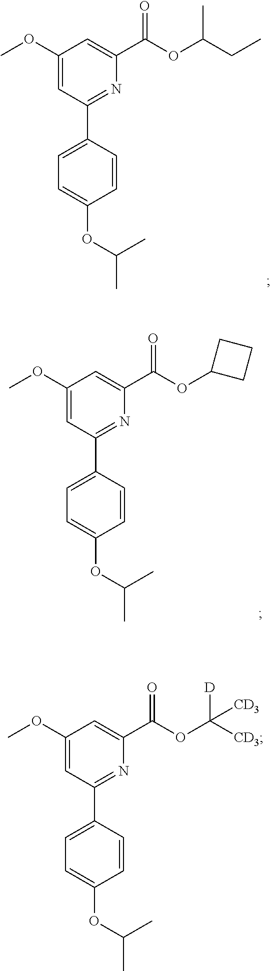

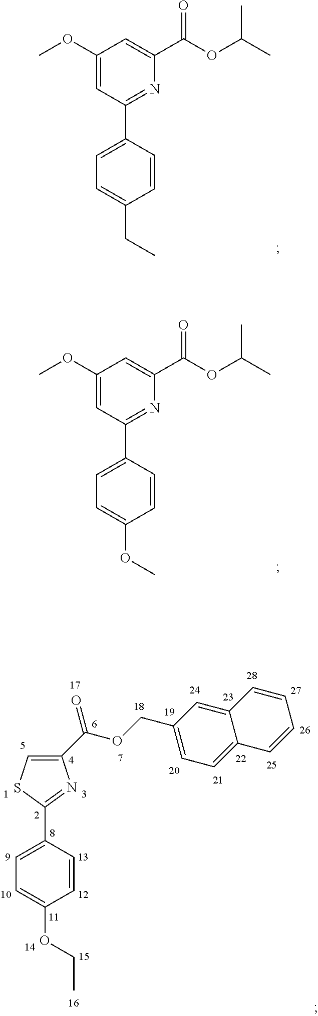

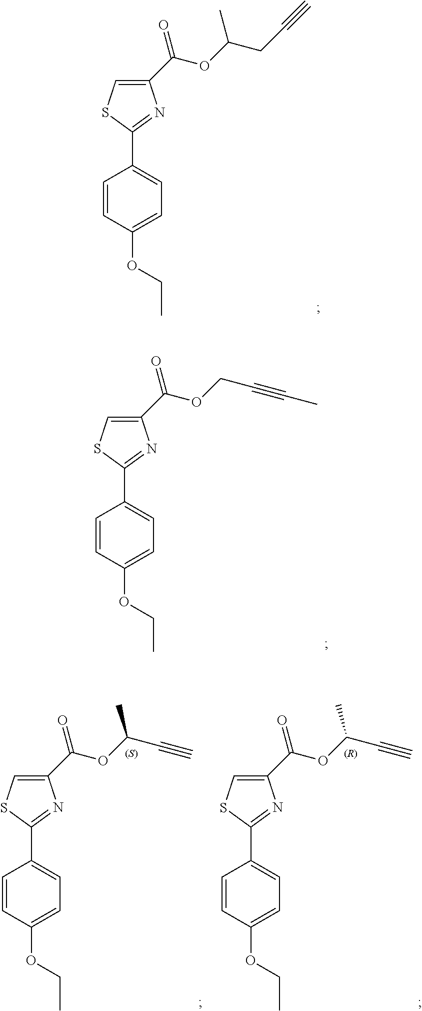

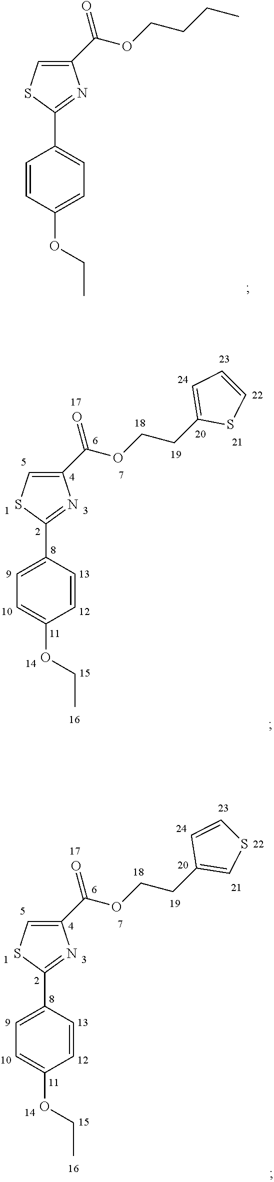 [SMILES: CCCCOC(=O)c1csc(-c2ccc(OCC)cc2)n1 ; CCOc1ccc(-c2nc(C(=O)OCCc3cccs3)cs2)cc1 ; CCOc1ccc(-c2nc(C(=O)OCCc3ccsc3)cs2)cc1]

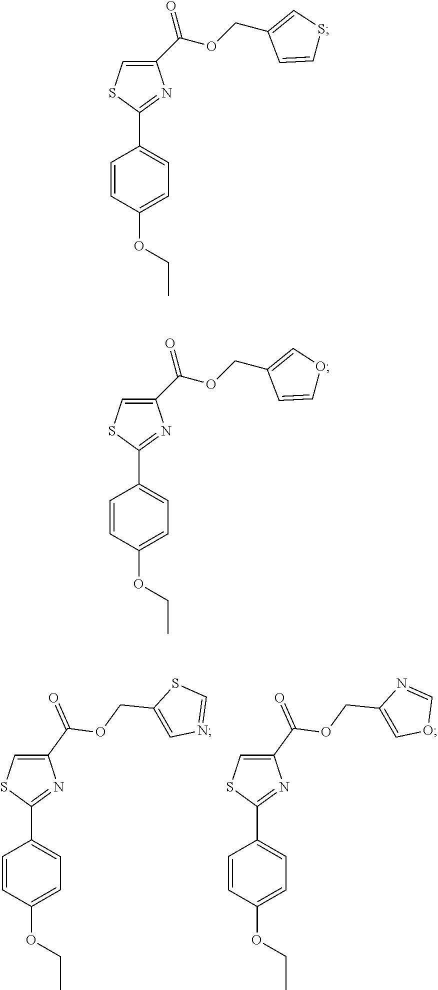

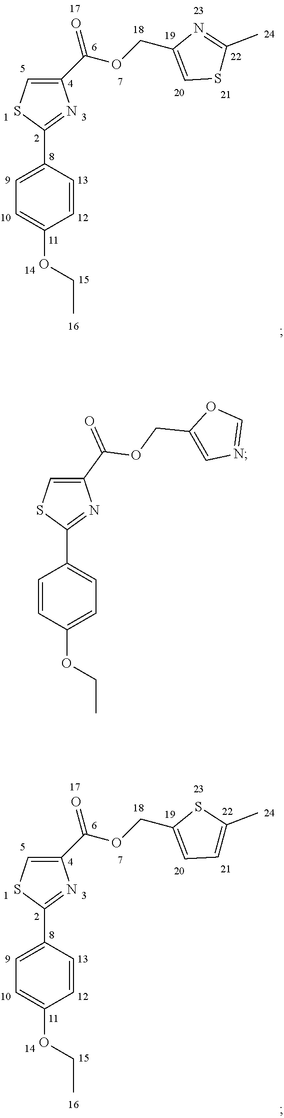 [SMILES: CCOc1ccc(-c2nc(C(=O)OCc3csc(C)n3)cs2)cc1 ; CCOc1ccc(-c2nc(C(=O)OCc3cnco3)cs2)cc1 ; CCOc1ccc(-c2nc(C(=O)OCc3ccc(C)s3)cs2)cc1]

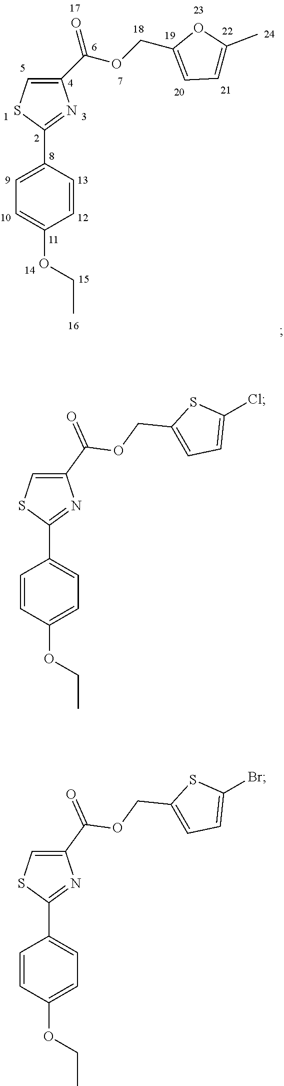 [SMILES: CCOc1ccc(-c2nc(C(=O)OCc3ccc(C)o3)cs2)cc1 ; CCOc1ccc(-c2nc(C(=O)OCc3ccc(Cl)s3)cs2)cc1 ; CCOc1ccc(-c2nc(C(=O)OCc3ccc(Br)s3)cs2)cc1]

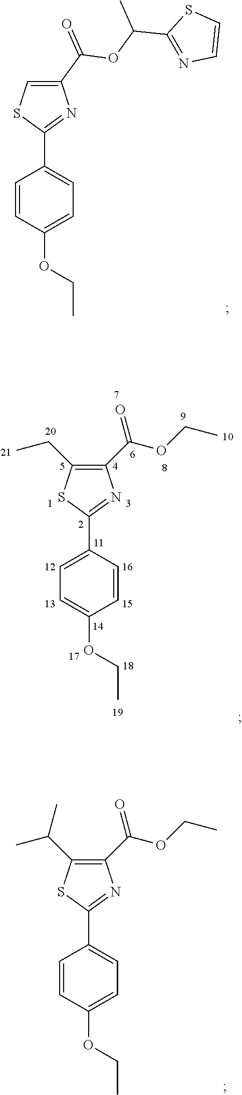

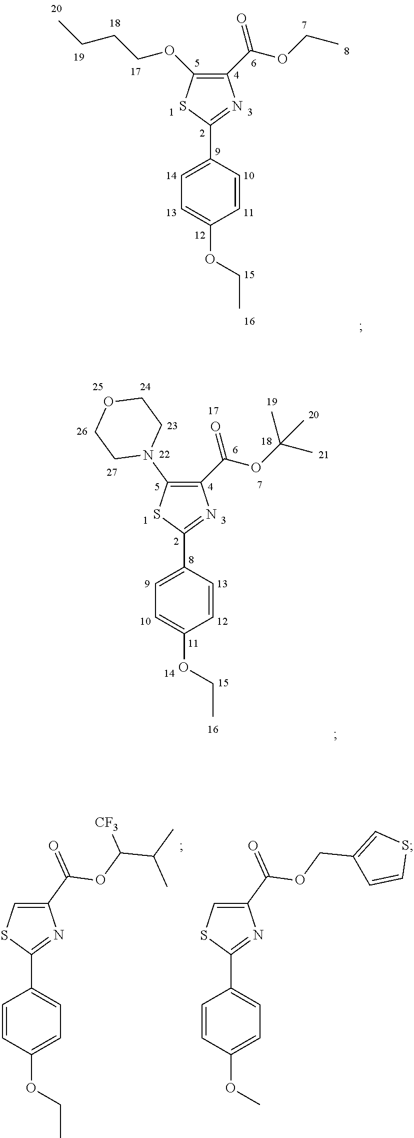 [SMILES: CCCCOc1sc(-c2ccc(OCC)cc2)nc1C(=O)OCC ; CCOc1ccc(-c2nc(C(=O)OC(C)(C)C)c(N3CCOCC3)s2)cc1 ; CCOc1ccc(-c2nc(C(=O)OC(C(C)C)C(F)(F)F)cs2)cc1 ; COc1ccc(-c2nc(C(=O)OCc3ccsc3)cs2)cc1]

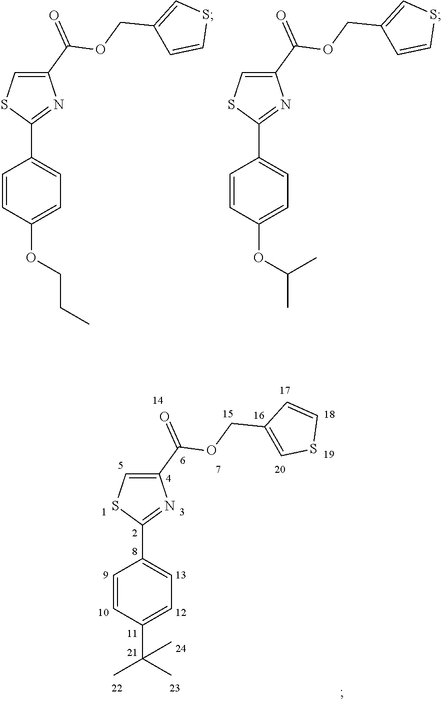

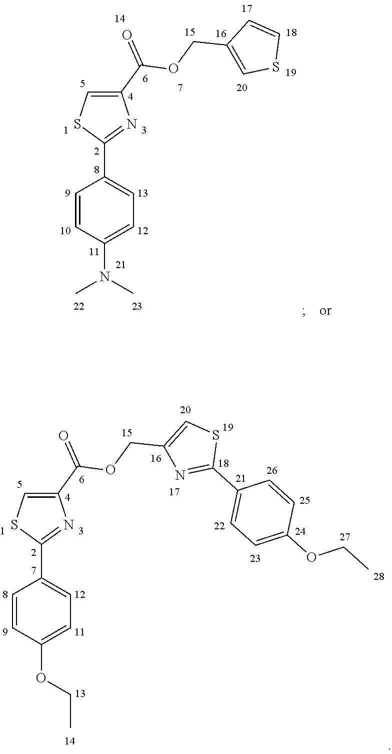 [SMILES: CN(C)c1ccc(-c2nc(C(=O)OCc3ccsc3)cs2)cc1 ; CCOc1ccc(-c2nc(COC(=O)c3csc(-c4ccc(OCC)cc4)n3)cs2)cc1]

13. A compound of the following formula 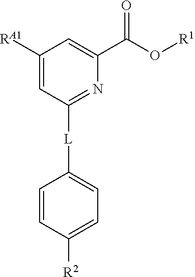
or a pharmaceutically acceptable salt or solvate thereof,
wherein:
L is selected from a covalent bond, C₁₋₅ alkylene, C₂₋₅ alkenylene, and C₂₋₅ alkynylene, wherein one —CH₂— unit comprised in said C₁₋₅ alkylene, said C₂₋₅ alkenylene or said C₂₋₅ alkynylene is optionally replaced by —O—;
R¹ is selected from C₁₋₁₀ alkyl, C₂₋₁₀ alkenyl, C₂₋₁₀ alkynyl, carbocyclyl, and heterocyclyl, wherein said alkyl, said alkenyl and said alkynyl are each optionally substituted with one or more groups R^{Alk}, and wherein said carbocyclyl and said heterocyclyl are each optionally substituted with one or more groups R^{Cyc};
R² is selected from hydrogen, C₁₋₁₀ alkyl, C₂₋₁₀ alkenyl, C₂₋₁₀ alkynyl, —(C₀₋₄ alkylene)-OH, —(C₀₋₄ alkylene)-O(C₁₋₁₀ alkyl), —(C₀₋₄ alkylene)-O(C₁₋₁₀ alkylene)-OH, —(C₀₋₄ alkylene)-O(C₁₋₁₀ alkylene)-O(C₁₋₅ alkyl), —(C₀₋₄ alkylene)-O(C₁₋₅ alkylene)-O(C₁₋₅ alkylene)-OH, —(C₀₋₄ alkylene)-O(C₁₋₅ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(C₁₋₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—CO—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}R^{X}, wherein said C₁₋₁₀ alkyl, said C₂₋₁₀ alkenyl, said C₂₋₁₀ alkynyl, each alkyl moiety in any of the aforementioned groups, and each alkylene moiety in any of the aforementioned groups are each optionally substituted with one or more groups R^{Alk}, and wherein the carbocyclyl moiety in said -L^{X}-carbocyclyl and the heterocyclyl moiety in said -L^{X}-heterocyclyl are each optionally substituted with one or more groups R^{Cyc};
R^{A1} is selected from hydrogen, C₁₋₅ alkyl, C₂₋₅ alkenyl, C₂₋₅ alkynyl, —(C₀₋₄ alkylene)-OH, —(C₀₋₄ alkylene)-O(C₁₋₅ alkyl), —(C₀₋₄ alkylene)-O(C₁₋₅ alkylene)-OH, —(C₀₋₄ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(C₁₋₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—CO—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups R^{Cyc};
each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups R^{Cyc};
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —C(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl);
each L^{X} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —ON, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{X} is independently selected from hydrogen, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), carbocyclyl, and heterocyclyl, wherein said carbocyclyl and said heterocyclyl are each optionally substituted with one or more groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(CO₁₋₅ alkyl), and —SO—(C₁₋₅ alkyl);
and further wherein the following compounds are excluded:
ethyl 6-(4-methoxyphenyl)-2-pyridinecarboxylate;
ethyl 6-(4-hydroxyphenyl)-2-pyridinecarboxylate; and
ethyl 6-(4-{[(3-fluorophenyl)methyl]oxy}phenyl)-2-pyridinecarboxylate.

14. The compound of claim 13, wherein L is a covalent bond.

15. The compound of claim 13 or 14, wherein R¹ is selected from C₁₋₆ alkyl, C₂₋₆ alkenyl, C₂₋₆ alkynyl, cycloalkyl, and heterocycloalkyl.

16. The compound of any one of claims 13 to 15, wherein R¹ is selected from ethyl, isopropyl, —CH₂—CH═CH₂, —CH₂—C(CH₃)═CH₂, —CH(CH₃)—C≡CH, and cyclopropyl.

17. The compound of any one of claims 13 to 16, wherein R¹ is ethyl or isopropyl.

18. The compound of any one of claims 13 to 17, wherein R² is selected from C₁₋₁₀ alkyl, —O(C₁₋₁₀ alkyl), —(C₁₋₄ alkylene)-O(C₁₋₁₀ alkyl), —O(C₁₋₁₀ alkylene)-O(C₁₋₅ alkyl), —(C₁₋₄ alkylene)-O(C₁₋₁₀ alkylene)-O(C₁₋₅ alkyl), —O(C₁₋₅ alkylene)-O(C₁₋₅ alkylene)-O(C₁₋₅ alkyl), —(C₁₋₄ alkylene)-O(C₁₋₅ alkylene)-O(C₁₋₅ alkylene)-O(C₁₋₅ alkyl), —O(C₂₋₄ alkenyl), —S(C₁₋₅ alkyl), —COO—(C₁₋₅ alkyl), —CO—N(C₁₋₅ alkyl)(C₁₋₅ alkyl), —CO—N(C₁₋₅ alkyl)-O—(C₁₋₅ alkyl), —SO₂—(C₁₋₅ alkyl), halogen, C₁₋₅ haloalkyl, —O—(C₁₋₅ haloalkyl), -L^{X}-aryl, -L^{X}-cycloalkyl, -L^{X}-heteroaryl, and -L^{X}-heterocycloalkyl, wherein the aryl moiety in said -L^{X}-aryl, the cycloalkyl moiety in said -L^{X}-cycloalkyl, the heteroaryl moiety in said -L^{X}-heteroaryl, and the heterocycloalkyl moiety in said -L^{X}-heterocycloalkyl are each optionally substituted with one or more groups R^{Cyc}.

19. The compound of any one of claims 13 to 18, wherein R² is selected from —CH₂CH₃, —(CH₂)₂CH₃, —(CH₂)₃CH₃, —(CH₂)₄CH₃, —(CH₂)₅CH₃, —(CH₂)₆CH₃, —O—CH₂CH₃, —O—(CH₂)₂CH₃, —O—(CH₂)₃CH₃, —O—(CH₂)₄CH₃, —O—(CH₂)₅CH₃, —O—(CH₂)₆CH₃, —CH₂—O—CH₃, and —CH(—CH₃)—O—CH₃, preferably wherein R² is —O—CH₂CH₃.

20. The compound of any one of claims 13 to 19, wherein R^{A1} is selected from hydrogen, —CH₃, —OCH₃, —CO—CH₃, and —I.

21. The compound of any one of claims 13 to 20, wherein R^{A1} is hydrogen.

22. The compound of claim 13, wherein said compound is any one of the following compounds, or a pharmaceutically acceptable salt or solvate thereof:

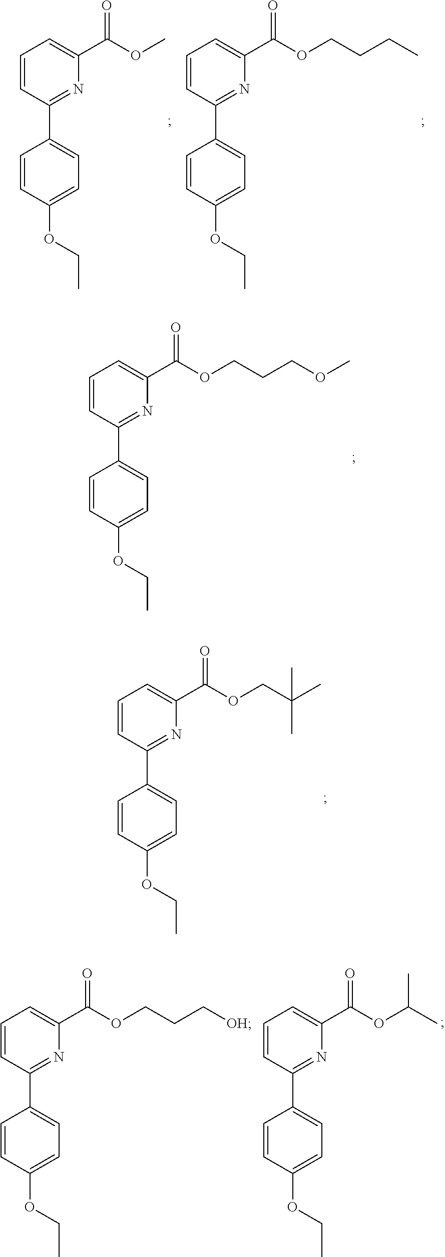

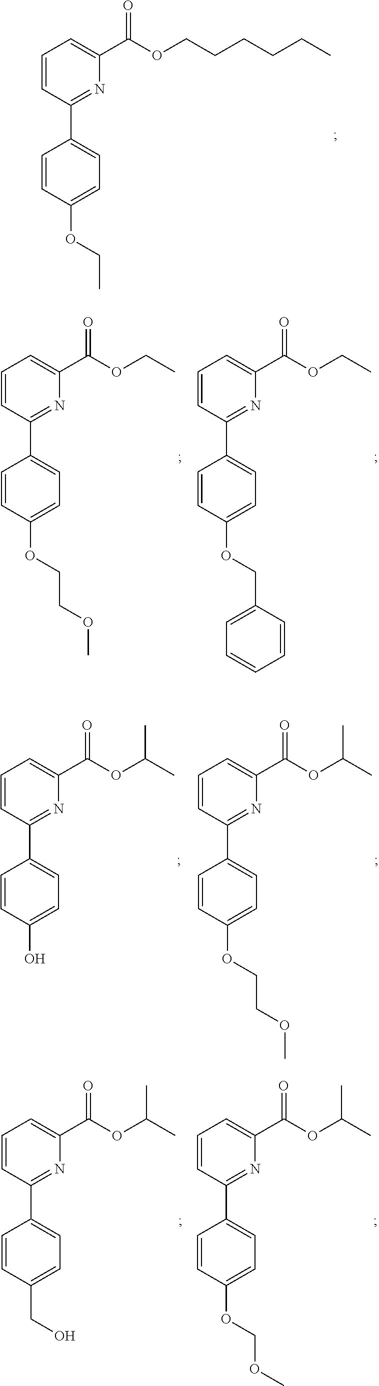

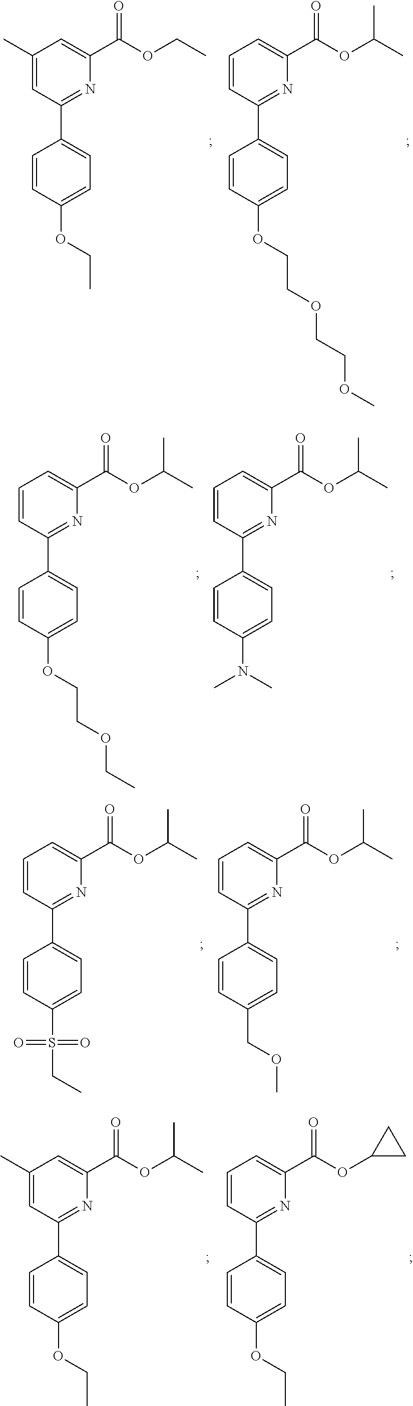

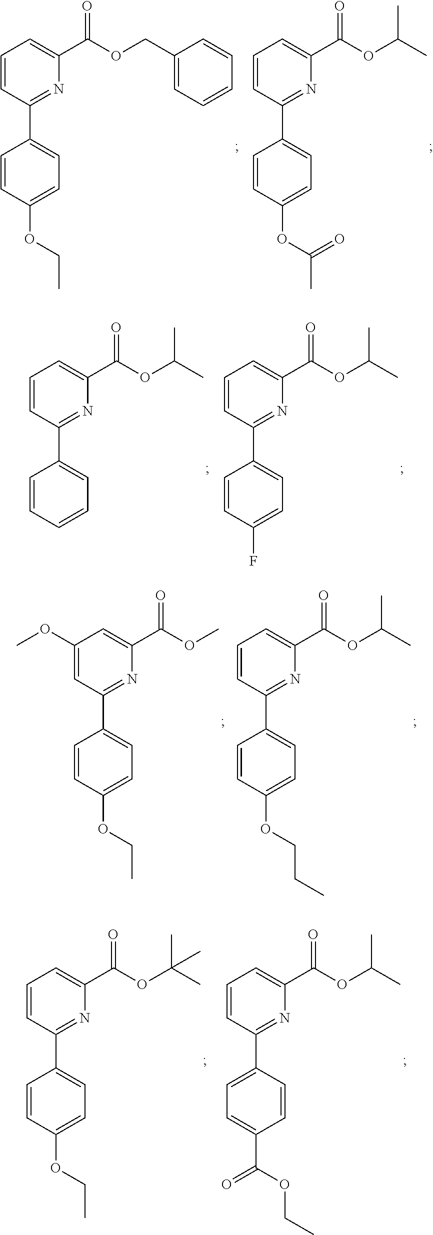

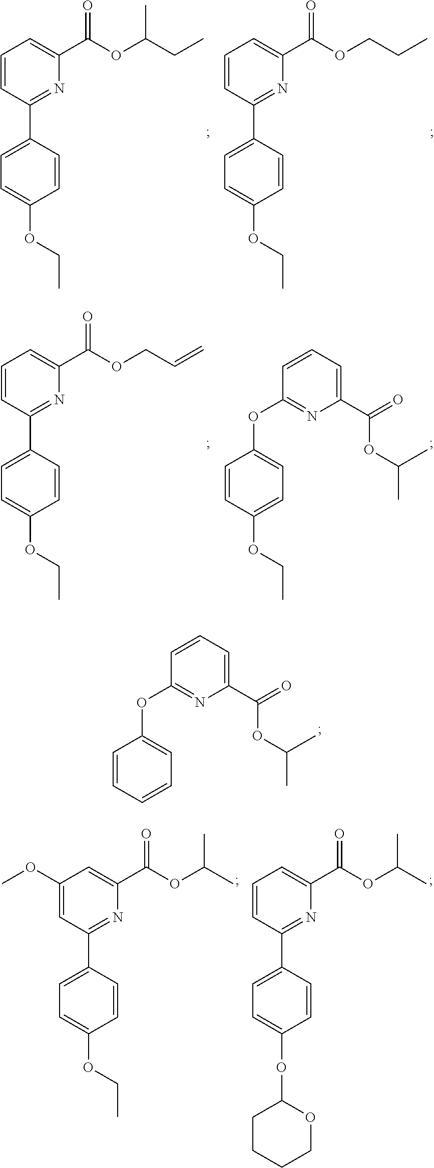

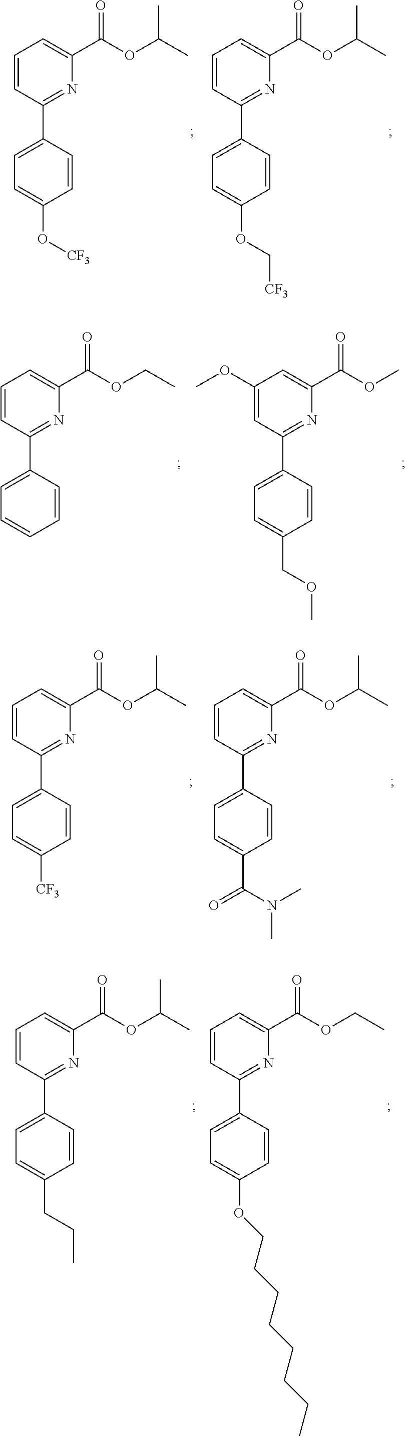

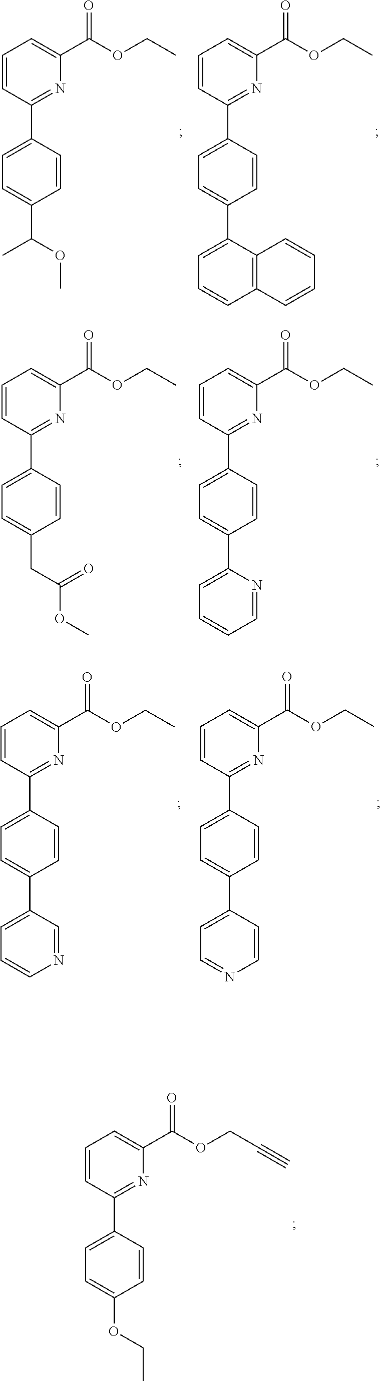

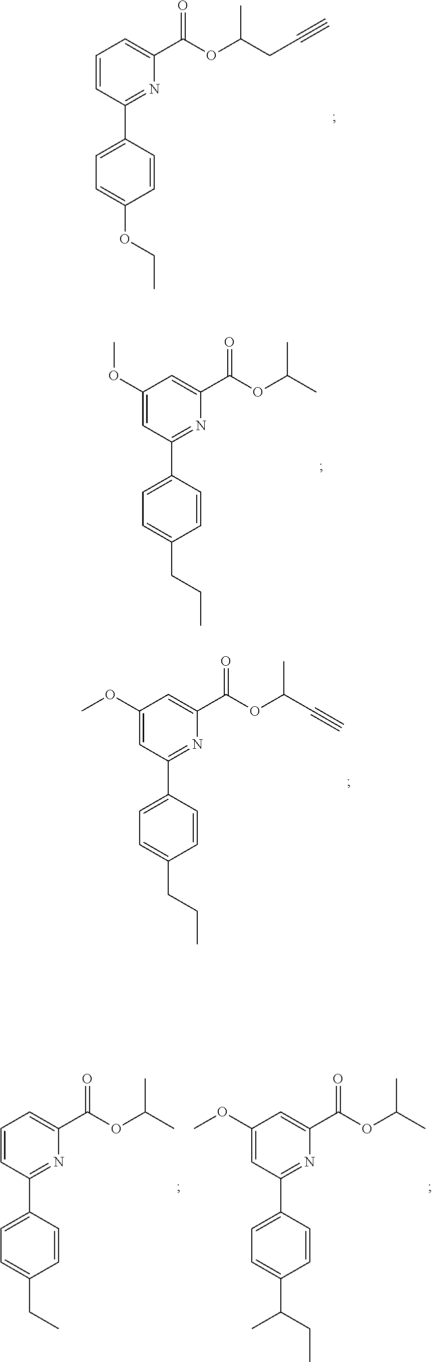

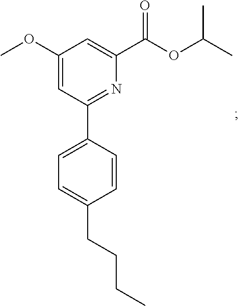 [SMILES: CCCCc1ccc(-c2cc(OC)cc(C(=O)OC(C)C)n2)cc1]

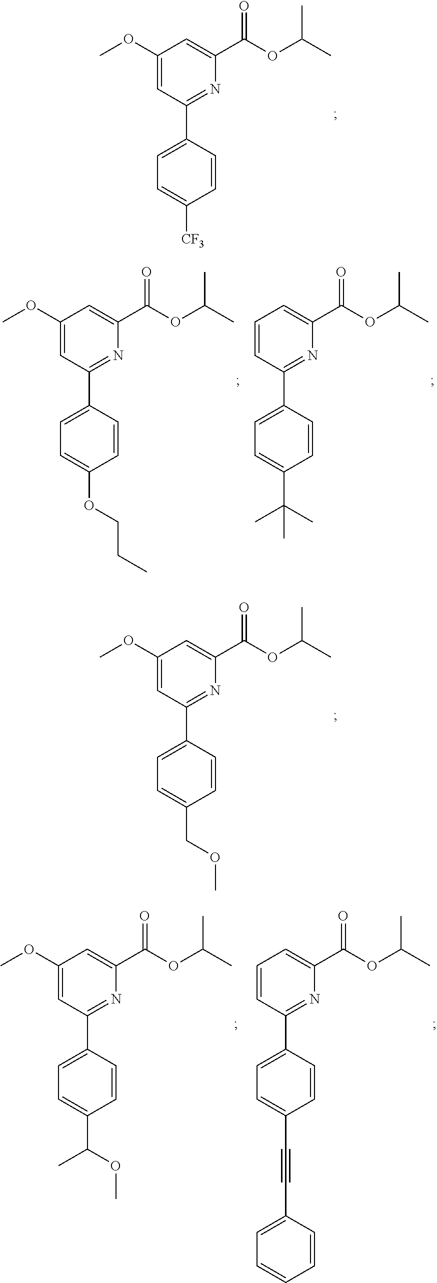

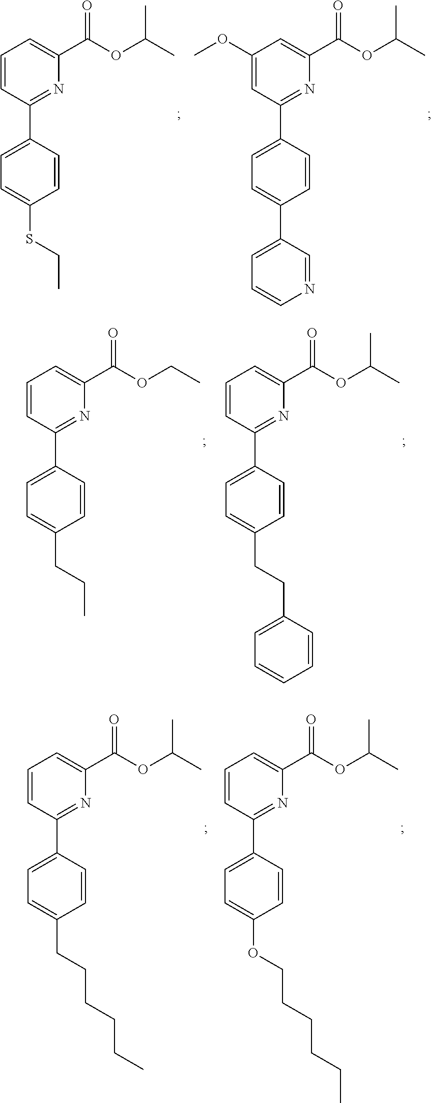

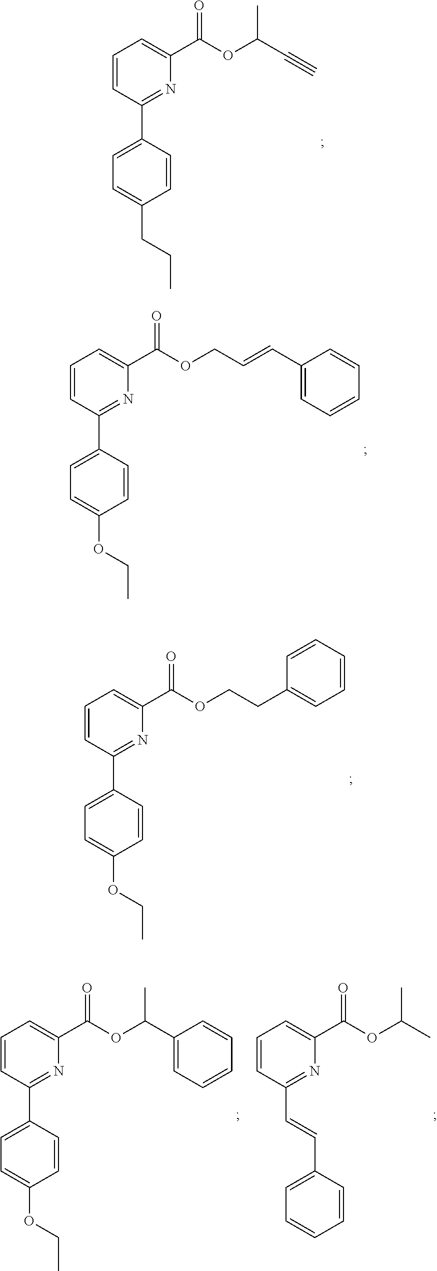

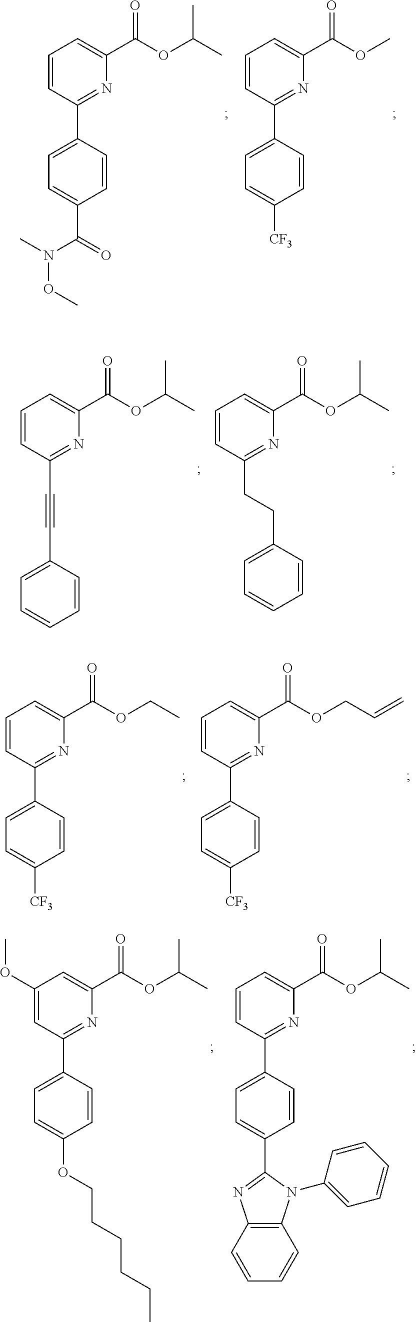

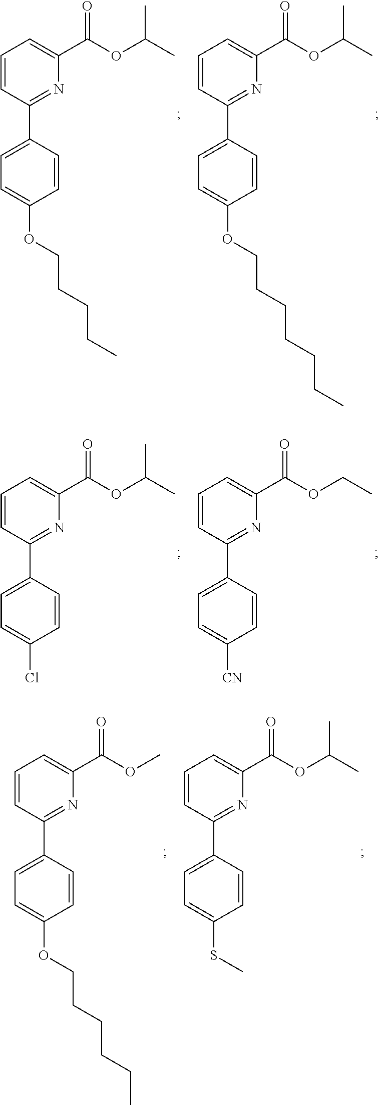

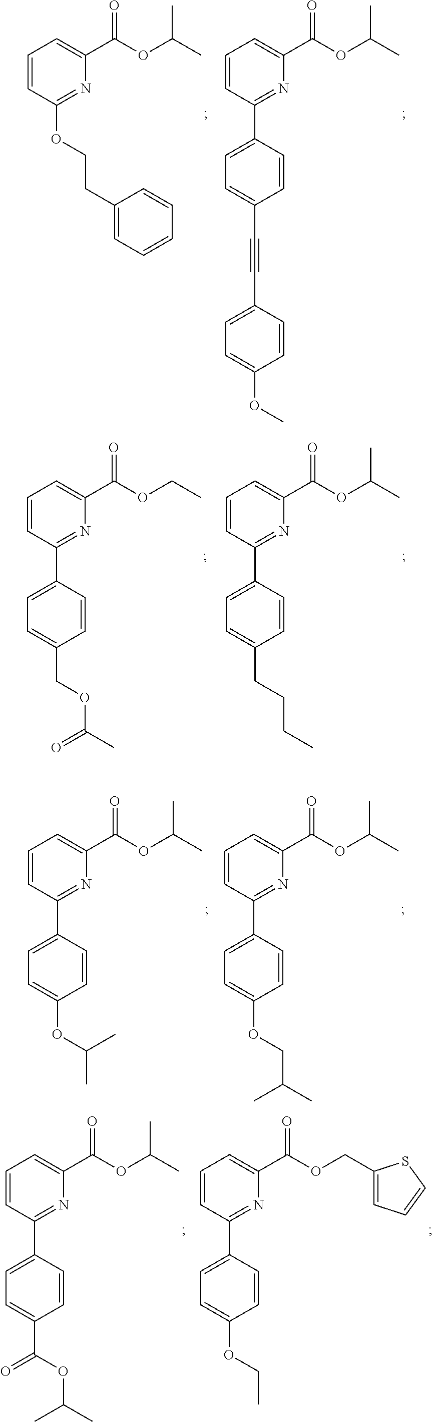

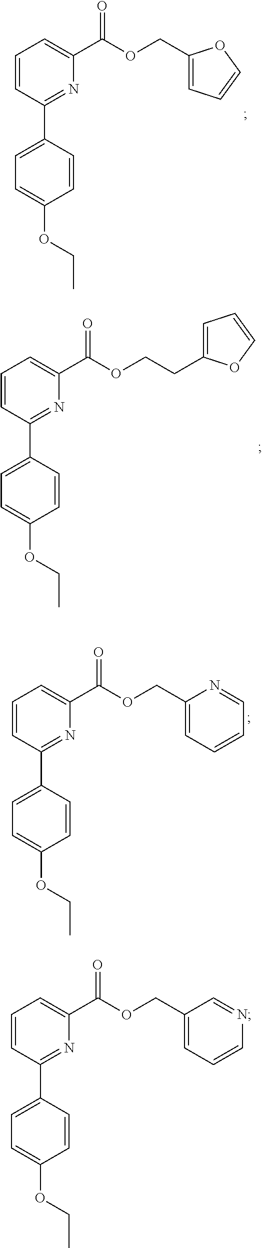

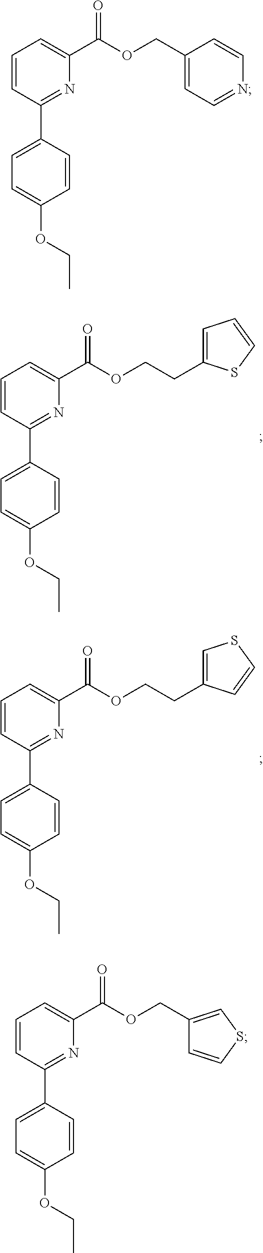

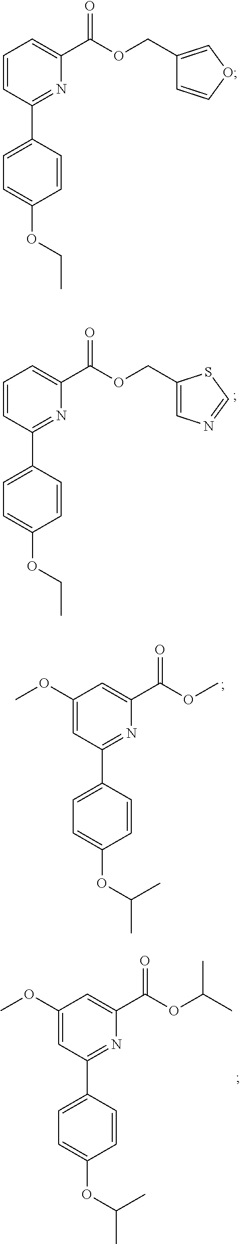

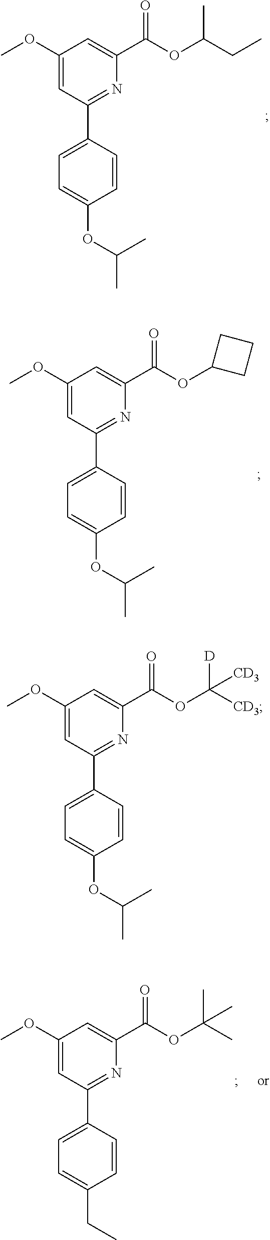

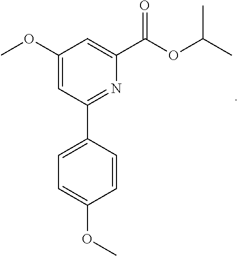 [SMILES: COc1ccc(-c2cc(OC)cc(C(=O)OC(C)C)n2)cc1]

23. A compound of the following formula 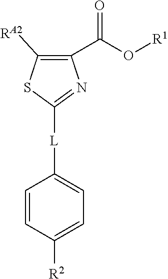
or a pharmaceutically acceptable salt or solvate thereof,
wherein:
L is selected from a covalent bond, C₁₋₅ alkylene, C₂₋₅ alkenylene, and C₂₋₅ alkynylene, wherein one —CH₂— unit comprised in said C₁₋₅ alkylene, said C₂₋₅ alkenylene or said C₂₋₅ alkynylene is optionally replaced by —O—;
R¹ is selected from C₁₋₁₀ alkyl, C₂₋₁₀ alkenyl, C₂₋₁₀ alkynyl, carbocyclyl, and heterocyclyl, wherein said alkyl, said alkenyl and said alkynyl are each optionally substituted with one or more groups R^{Alk}, and wherein said carbocyclyl and said heterocyclyl are each optionally substituted with one or more groups R^{Cyc};
R² is selected from hydrogen, C₁₋₁₀ alkyl, —(C₀₋₄ alkylene)-O(C₁₋₁₀ alkyl), —(C₀₋₄ alkylene)-O(C₁₋₁₀ alkylene)-O(C₁₋₅ alkyl), —(C₀₋₄ alkylene)-O(C₁₋₅ alkylene)-O(C₁₋₅ alkylene)-O(C₁₋₅ alkyl), —O(C₂₋₄ alkenyl), —(C₀₋₄ alkylene)-S(C₁₋₅ alkyl), —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-SO₂—(C₁₋₅ alkyl), halogen, C₁₋₅ haloalkyl, —(C₀₋₄ alkylene)-O—(C₁₋₅ fluoroalkyl), -L^{X}-aryl, -L^{X}-heteroaryl, cycloalkyl, and heterocycloalkyl, wherein the aryl moiety in said -L^{X}-aryl, the heteroaryl moiety in said -L^{X}-heteroaryl, said cycloalkyl, and said heterocycloalkyl are each optionally substituted with one or more groups R^{Cyc};
R^{A2} is selected from hydrogen, C₁₋₅ alkyl, C₂₋₅ alkenyl, C₂₋₅ alkynyl, —(C₀₋₄ alkylene)-OH, —(C₀₋₄ alkylene)-O(C₁₋₅ alkyl), —(C₀₋₄ alkylene)-O(C₁₋₅ alkylene)-OH, —(C₀₋₄ alkylene)-O(C₁₋₅ alkylene)-O(C₁₋₅ alkyl), —(C₀₋₄ alkylene)-SH, —(C₀₋₄ alkylene)-S(C₁₋₅ alkyl), —(C₀₋₄ alkylene)-NH₂, —(C₀₋₄ alkylene)-NH(C₁₋₅ alkyl), —(C₀₋₄ alkylene)-N(C₁₋₅ alkyl)(C₁₋₅ alkyl), halogen, C₁₋₅ haloalkyl, —(C₀₋₄ alkylene)-O—(C₁₋₅ haloalkyl), —(C₀₋₄ alkylene)-CN, —(C₀₋₄ alkylene)-CHO, —(C₀₋₄ alkylene)-CO—(C₁₋₅ alkyl), —(C₀₋₄ alkylene)-COOH, —(C₀₋₄ alkylene)-CO—O—(C₁₋₅ alkyl), —(C₀₋₄ alkylene)-O—CO—(C₁₋₅ alkyl), —(C₀₋₄ alkylene)-CO—NH₂, —(C₀₋₄ alkylene)-CO—NH(C₁₋₅ alkyl), —(C₀₋₄ alkylene)-CO—N(C₁₋₅ alkyl)(C₁₋₅ alkyl), —(C₀₋₄ alkylene)-CO—NH—O—(C₁₋₅ alkyl), —(C₀₋₄ alkylene)-CO—N(C₁₋₅ alkyl)-O—(C₁₋₅ alkyl), —(C₀₋₄ alkylene)-NH—CO—(C₁₋₅ alkyl), —(C₀₋₄ alkylene)-N(C₁₋₅ alkyl)-CO—(C₁₋₅ alkyl), —(C₀₋₄ alkylene)-NH—CO—O—(C₁₋₅ alkyl), —(C₀₋₄ alkylene)-N(C₁₋₅ alkyl)-CO—O—(C₁₋₅ alkyl), —(C₀₋₄ alkylene)-O—CO—NH—(C₁₋₅ alkyl), —(C₀₋₄ alkylene)-O—CO—N(C₁₋₅ alkyl)-(C₁₋₅ alkyl), —(C₀₋₄ alkylene)-SO₂—NH₂, —(C₀₋₄ alkylene)-SO₂—NH(C₁₋₅ alkyl), —(C₀₋₄ alkylene)-SO₂—N(C₁₋₅ alkyl)(C₁₋₅ alkyl), —(C₀₋₄ alkylene)-NH—SO₂—(C₁₋₅ alkyl), —(C₀₋₄ alkylene)-N(C₁₋₅ alkyl)-SO₂—(C₁₋₅ alkyl), —(C₀₋₄ alkylene)-SO₂—(C₁₋₅ alkyl), —(C₀₋₄ alkylene)-SO—(C₁₋₅ alkyl), -L^{X}-carbocyclyl, -L^{X}-heterocyclyl, and -L^{X}R^{X}, wherein the carbocyclyl moiety in said -L^{X}-carbocyclyl and the heterocyclyl moiety in said -L^{X}-heterocyclyl are each optionally substituted with one or more groups R^{Cyc};
each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups R^{Cyc};
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{X}-carbocyclyl, -L^{X}-heterocyclyl, and -L^{X}-R^{X}, wherein the carbocyclyl moiety in said -L^{X}-carbocyclyl and the heterocyclyl moiety in said -L^{X}-heterocyclyl are each optionally substituted with one or more groups independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —O(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl);
each L^{X} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{X} is independently selected from hydrogen, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —N—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), carbocyclyl, and heterocyclyl, wherein said carbocyclyl and said heterocyclyl are each optionally substituted with one or more groups independently selected from C₁₋₅ alkyl, C₂₋₅ alkenyl, C₂₋₅ alkynyl, —OH, —O(C₁₋₅ alkyl), —O(C₁₋₅ alkylene)-OH, —(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl);
and further wherein the following compounds are excluded:
methyl 2-phenylthiazole-4-carboxylate;
methyl 2-(4-ethoxyphenyl)thiazole-4-carboxylate;
dimethyl 2,2′-[oxybis(4,1-phenylene)]bis(thiazole-4-carboxylate);
dimethyl 2,2′-(1,4-phenylene)dithiazole-4-carboxylate;
ethyl 2-phenyl-5-chloro-thiazole-4-carboxylate;
ethyl 2-(4-methoxyphenyl)-5-chloro-thiazole-4-carboxylate;
ethyl 2-(phenylethynyl)-5-chloro-thiazole-4-carboxylate;
ethyl 2-phenyl-5-phenyl-thiazole-4-carboxylate;
ethyl 2-phenyl-5-vinyl-thiazole-4-carboxylate;
ethyl 2-phenyl-5-(2-pyridyl)-thiazole-4-carboxylate;
ethyl 2-phenyl-5-(phenylethynyl)-thiazole-4-carboxylate;
ethyl 2-(4-methoxyphenyl)-5-phenyl-thiazole-4-carboxylate;
2-phenyl-4-carbethoxythiazole;
2-(4′-methoxyphenyl)-4-carbethoxythiazole;
2-(4′-methylphenyl)-4-carbethoxythiazole;
2-(4′-carbomethoxyphenyl)-4-carbethoxythiazole;
2-(4′-chlorophenyl)-4-carbethoxythiazole;
2-benzyl-4-carbethoxythiazole; and
2-(2′-phenylethyl)-4-carbethoxythiazole.

24. The compound of claim 23, wherein L is a covalent bond.

25. The compound of claim 23 or 24, wherein R¹ is selected from C₁₋₆ alkyl, C₂₋₆ alkenyl, C₂₋₆ alkynyl, cycloalkyl, and heterocycloalkyl.

26. The compound of any one of claims 23 to 25, wherein R¹ is selected from ethyl, isopropyl, —CH₂—CH═CH₂, —CH₂—C(CH₃)═CH₂, —CH(CH₃)—C≡CH, and cyclopropyl.

27. The compound of any one of claims 23 to 26, wherein R¹ is ethyl or isopropyl.

28. The compound of any one of claims 23 to 27, wherein R² is selected from C₁₋₁₀ alkyl, —O(C₁₋₁₀ alkyl), —(C₁₋₄ alkylene)-O(C₁₋₁₀ alkyl), —O(C₁₋₁₀ alkylene)-O(C₁₋₅ alkyl), —(C₁₋₄ alkylene)-O(C₁₋₁₀ alkylene)-O(C₁₋₅ alkyl), —O(C₁₋₅ alkylene)-O(C₁₋₅ alkylene)-O(C₁₋₅ alkyl), —(C₁₋₄ alkylene)-O(C₁₋₅ alkylene)-O(C₁₋₅ alkylene)-O(C₁₋₅ alkyl), —O(C₂₋₄ alkenyl), —S(C₁₋₅ alkyl), —CO—(C₁₋₅ alkyl), —CO—N(C₁₋₅ alkyl)(C₁₋₅ alkyl), —CO—N(C₁₋₅ alkyl)-O—(C₁₋₅ alkyl), —SO₂—(C₁₋₅ alkyl), halogen, C₁₋₅ haloalkyl, —O—(C₁₋₅ fluoroalkyl), -L^{X}-aryl, -L^{X}-heteroaryl, cycloalkyl, and heterocycloalkyl, wherein the aryl moiety in said -L^{X}-aryl, the heteroaryl moiety in said -L^{X}-heteroaryl, said cycloalkyl and said heterocycloalkyl are each optionally substituted with one or more groups R^{Cyc}.

29. The compound of any one of claims 23 to 28, wherein R² is selected from —CH₂CH₃, —(CH₂)₂CH₃, —(CH₂)₃CH₃, —(CH₂)₄CH₃, —(CH₂)₅CH₃, —(CH₂)₆CH₃, —O—CH₂CH₃, —O—(CH₂)₂CH₃, —O—(CH₂)₃CH₃, —O—(CH₂)₄CH₃, —O—(CH₂)₅CH₃, —O—(CH₂)₆CH₃, —CH₂—O—CH₃, and —CH(—CH₃)—O—CH₃, preferably wherein R² is —O—CH₂CH₃.

30. The compound of any one of claims 23 to 29, wherein R^{A2} is selected from hydrogen, —CH₃, —OCH₃, —CO—CH₃, and —I.

31. The compound of any one of claims 23 to 30, wherein R^{A2} is hydrogen.

32. The compound of claim 23, wherein said compound is any one of the following compounds, or a pharmaceutically acceptable salt or solvate thereof:

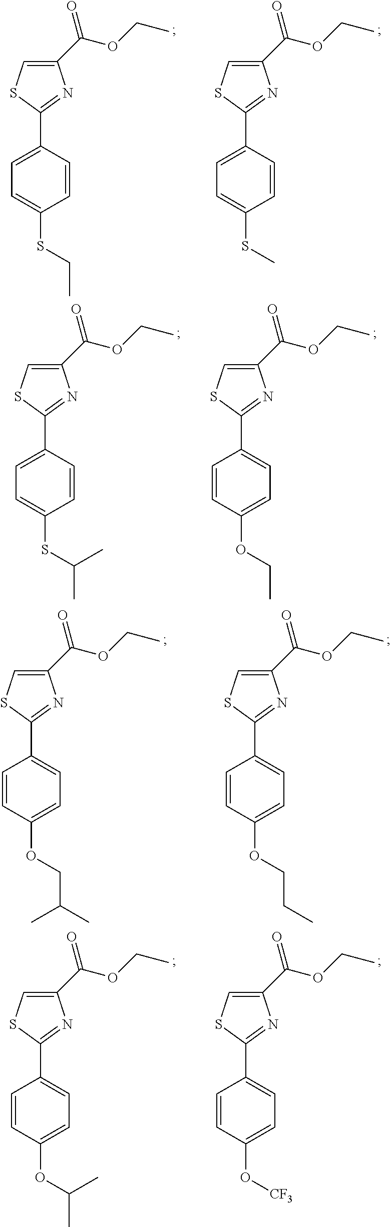

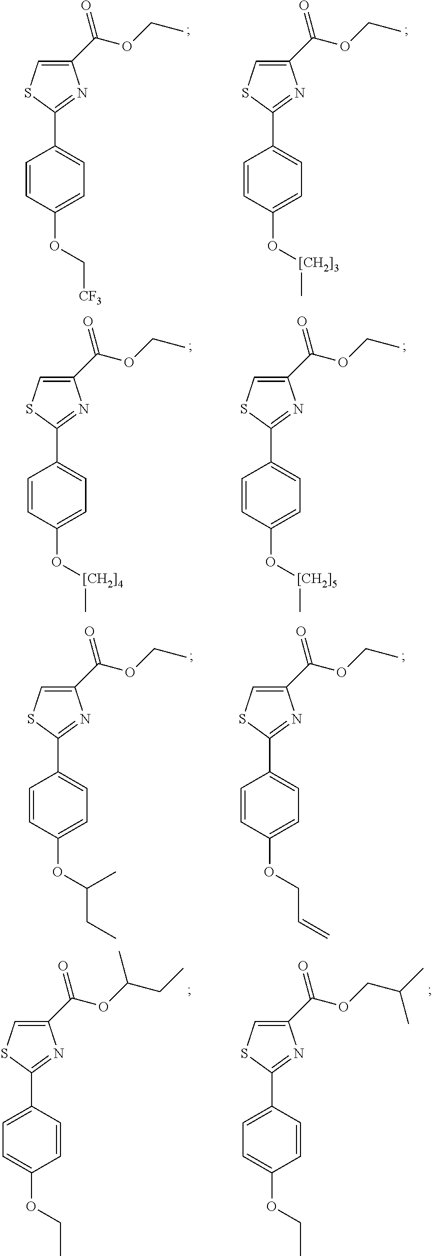

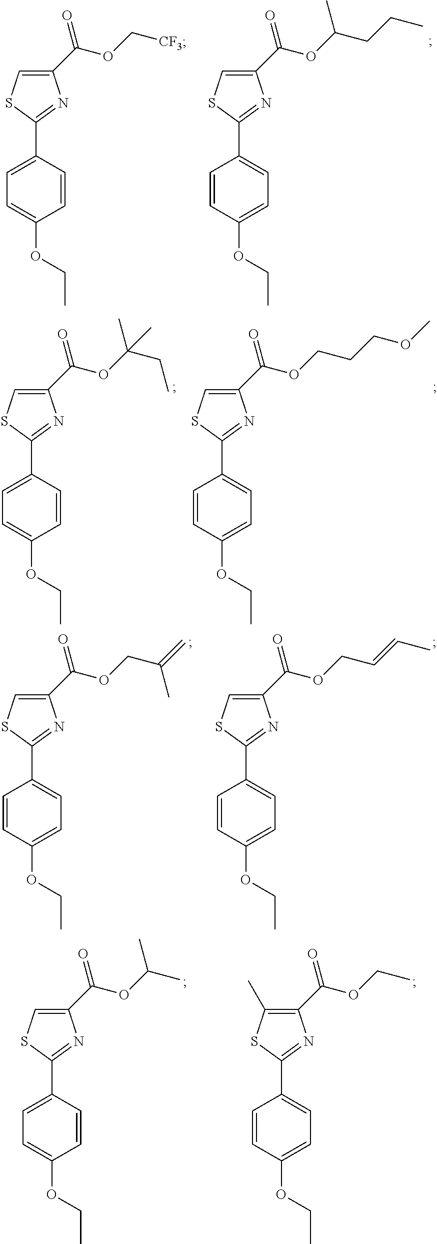

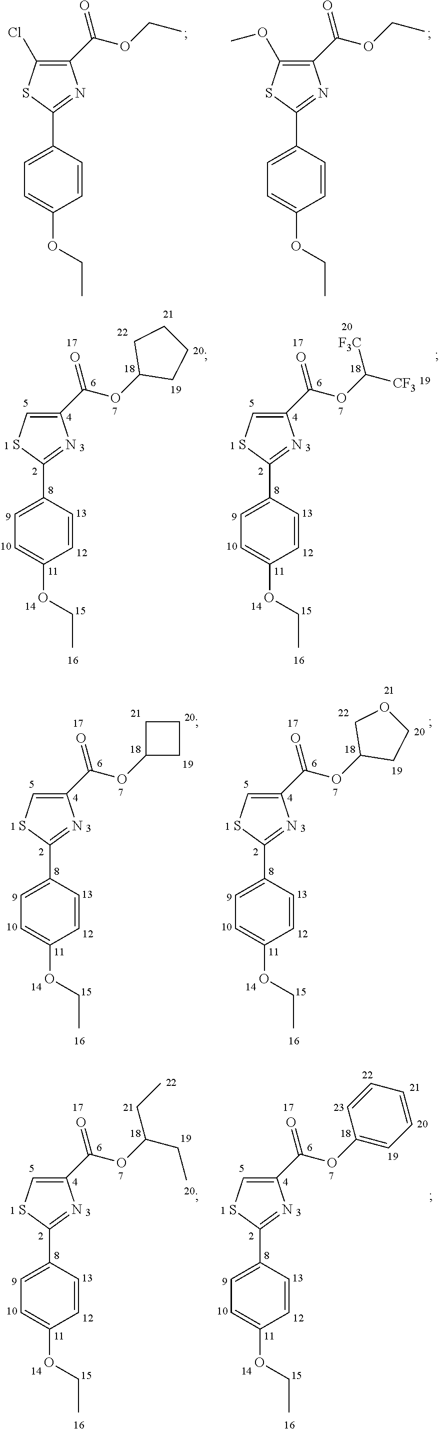

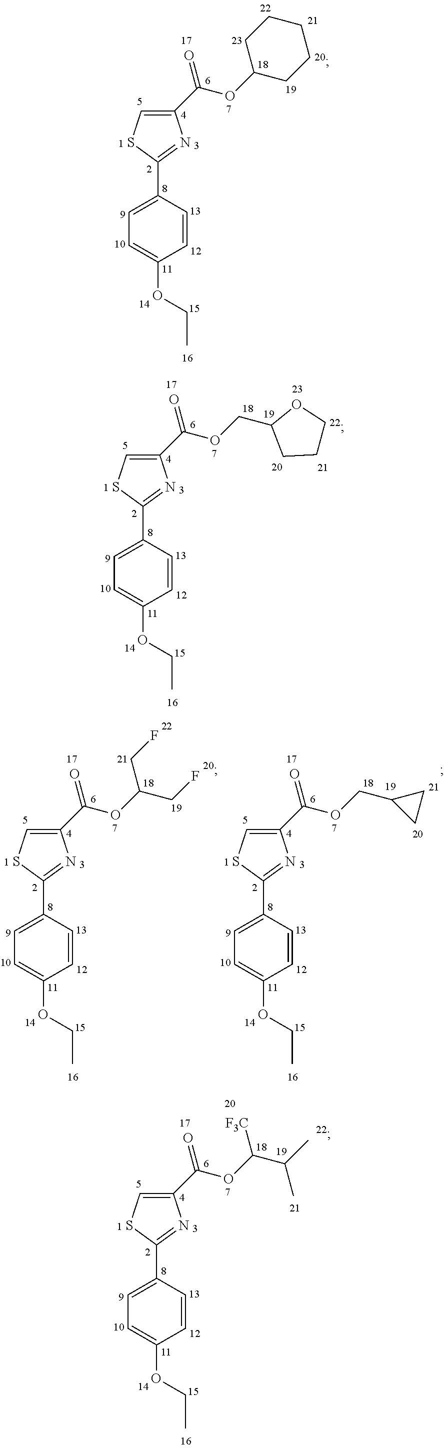 [SMILES: CCOc1ccc(-c2nc(C(=O)OC3CCCCC3)cs2)cc1 ; CCOc1ccc(-c2nc(C(=O)OCC3CCCO3)cs2)cc1 ; CCOc1ccc(-c2nc(C(=O)OC(CF)CF)cs2)cc1 ; CCOc1ccc(-c2nc(C(=O)OCC3CC3)cs2)cc1 ; CCOc1ccc(-c2nc(C(=O)OC(C(C)C)C(F)(F)F)cs2)cc1]

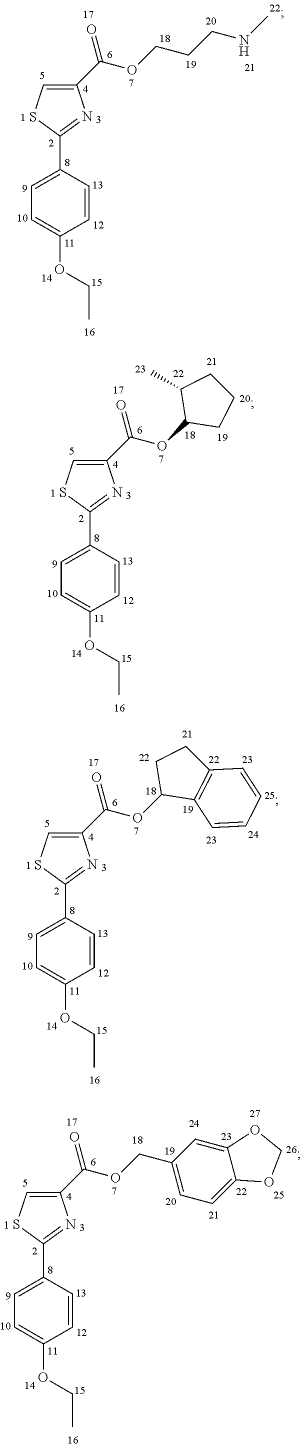

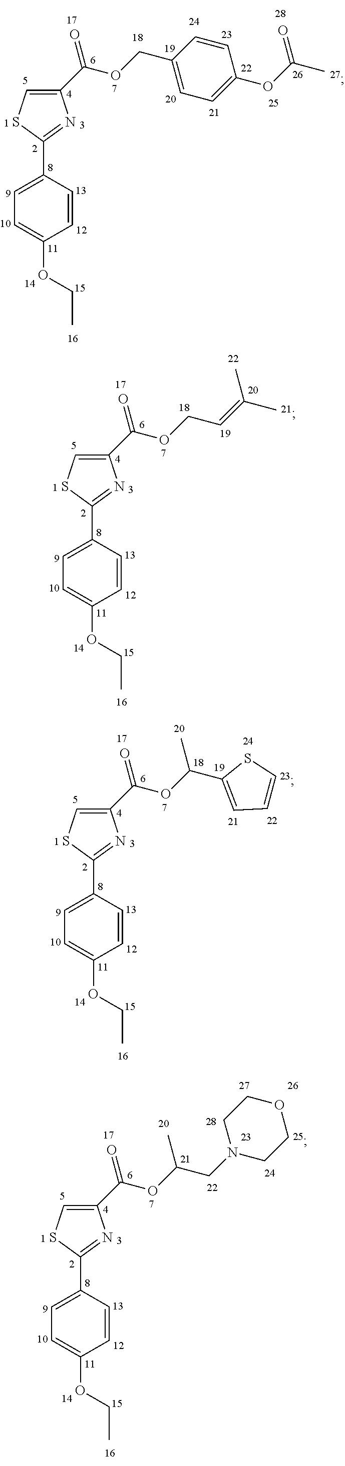 [SMILES: CCOc1ccc(-c2nc(C(=O)OCc3ccc(OC(C)=O)cc3)cs2)cc1 ; CCOc1ccc(-c2nc(C(=O)OCC=C(C)C)cs2)cc1 ; CCOc1ccc(-c2nc(C(=O)OC(C)c3cccs3)cs2)cc1 ; CCOc1ccc(-c2nc(C(=O)OC(C)CN3CCOCC3)cs2)cc1]

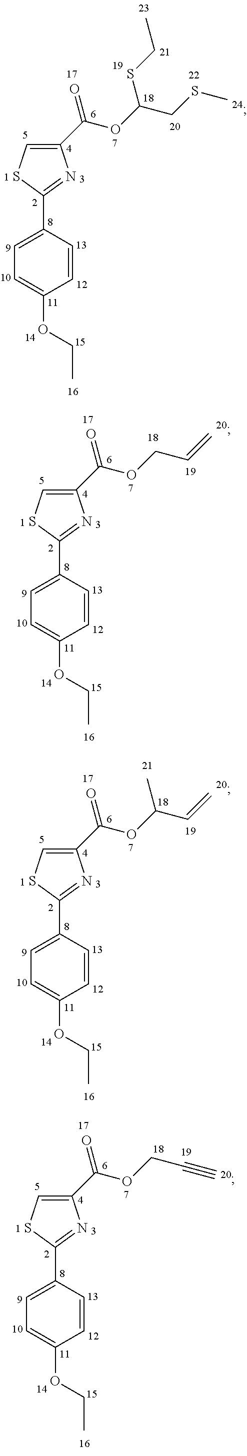 [SMILES: CCOc1ccc(-c2nc(C(=O)OC(CSC)SCC)cs2)cc1 ; C=CCOC(=O)c1csc(-c2ccc(OCC)cc2)n1 ; C=CC(C)OC(=O)c1csc(-c2ccc(OCC)cc2)n1 ; C#CCOC(=O)c1csc(-c2ccc(OCC)cc2)n1]

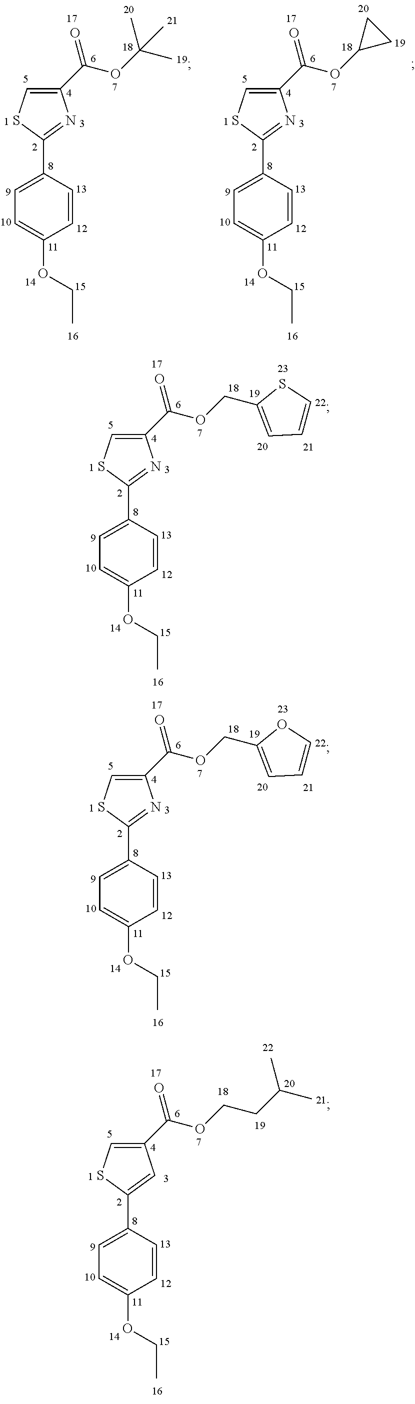 [SMILES: CCOc1ccc(-c2nc(C(=O)OC(C)(C)C)cs2)cc1 ; CCOc1ccc(-c2nc(C(=O)OC3CC3)cs2)cc1 ; CCOc1ccc(-c2nc(C(=O)OCc3cccs3)cs2)cc1 ; CCOc1ccc(-c2nc(C(=O)OCc3ccco3)cs2)cc1 ; CCOc1ccc(-c2cc(C(=O)OCCC(C)C)cs2)cc1]

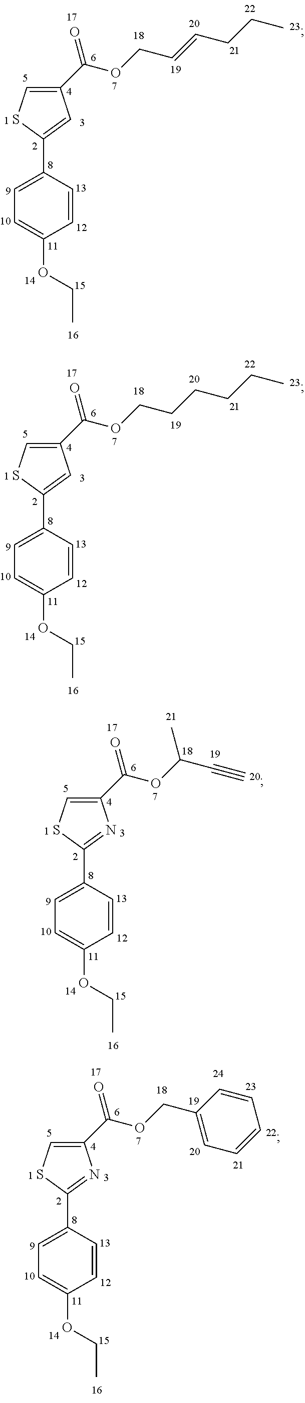 [SMILES: CCC/C=C/COC(=O)c1csc(-c2ccc(OCC)cc2)c1 ; CCCCCCOC(=O)c1csc(-c2ccc(OCC)cc2)c1 ; C#CC(C)OC(=O)c1csc(-c2ccc(OCC)cc2)n1 ; CCOc1ccc(-c2nc(C(=O)OCc3ccccc3)cs2)cc1]

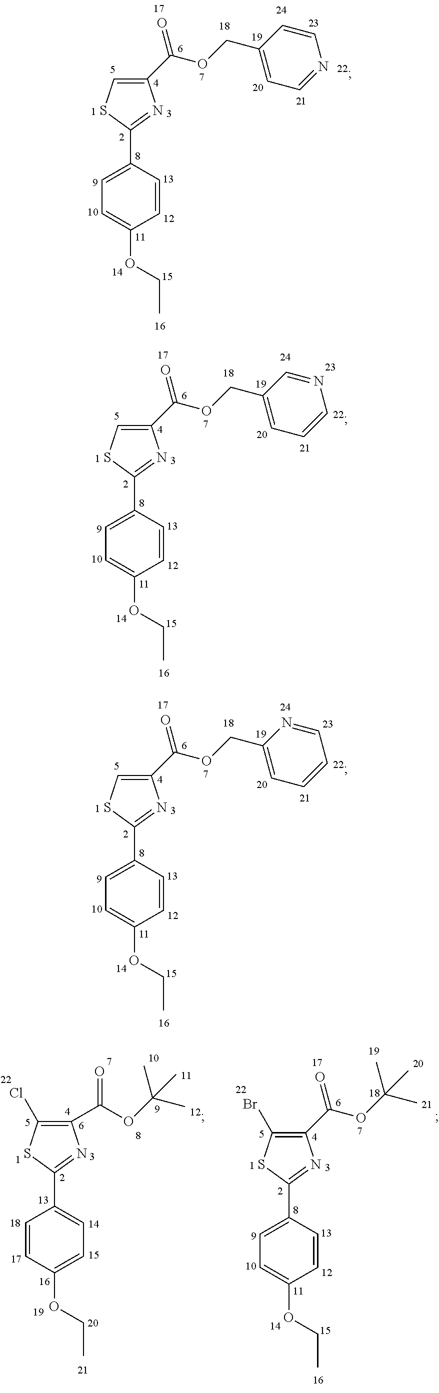 [SMILES: CCOc1ccc(-c2nc(C(=O)OCc3ccncc3)cs2)cc1 ; CCOc1ccc(-c2nc(C(=O)OCc3cccnc3)cs2)cc1 ; CCOc1ccc(-c2nc(C(=O)OCc3ccccn3)cs2)cc1 ; CCOc1ccc(-c2nc(C(=O)OC(C)(C)C)c(Cl)s2)cc1 ; CCOc1ccc(-c2nc(C(=O)OC(C)(C)C)c(Br)s2)cc1]

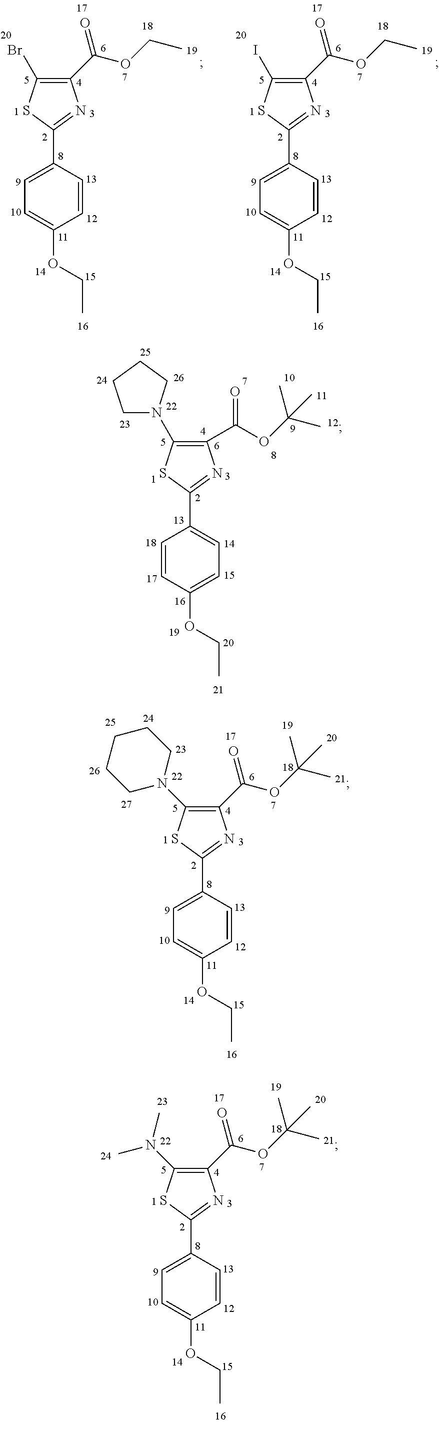 [SMILES: CCOC(=O)c1nc(-c2ccc(OCC)cc2)sc1Br ; CCOC(=O)c1nc(-c2ccc(OCC)cc2)sc1I ; CCOc1ccc(-c2nc(C(=O)OC(C)(C)C)c(N3CCCC3)s2)cc1 ; CCOc1ccc(-c2nc(C(=O)OC(C)(C)C)c(N3CCCCC3)s2)cc1 ; CCOc1ccc(-c2nc(C(=O)OC(C)(C)C)c(N(C)C)s2)cc1]

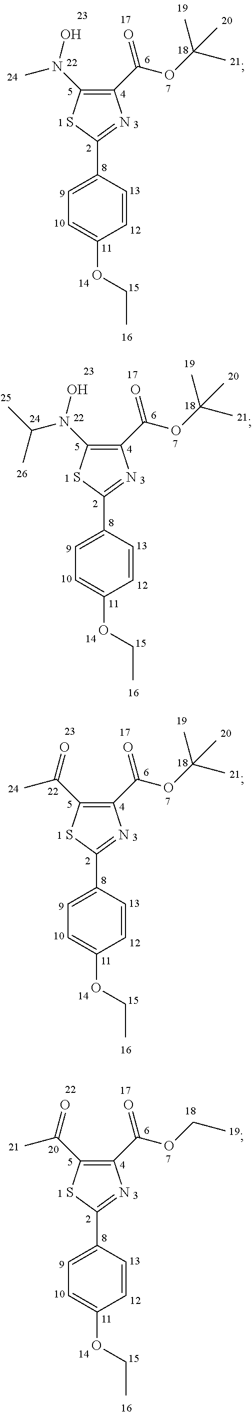

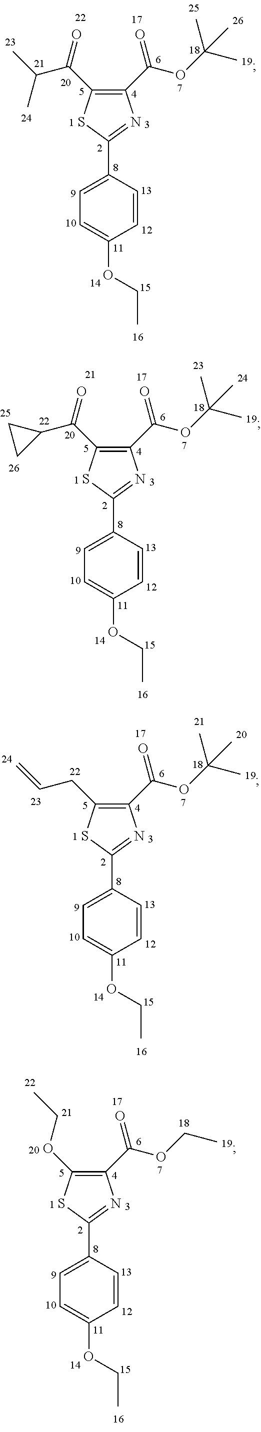

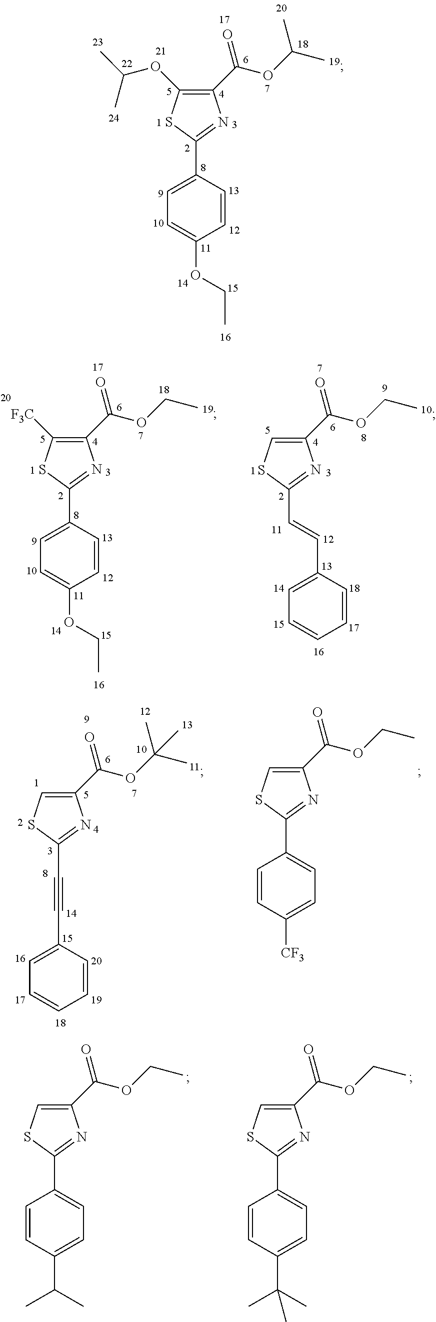

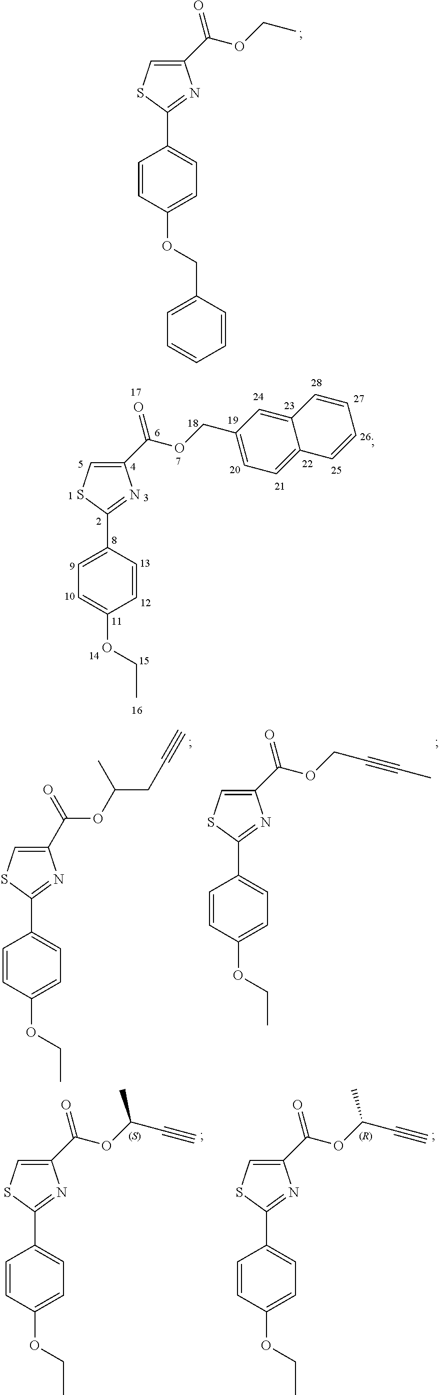

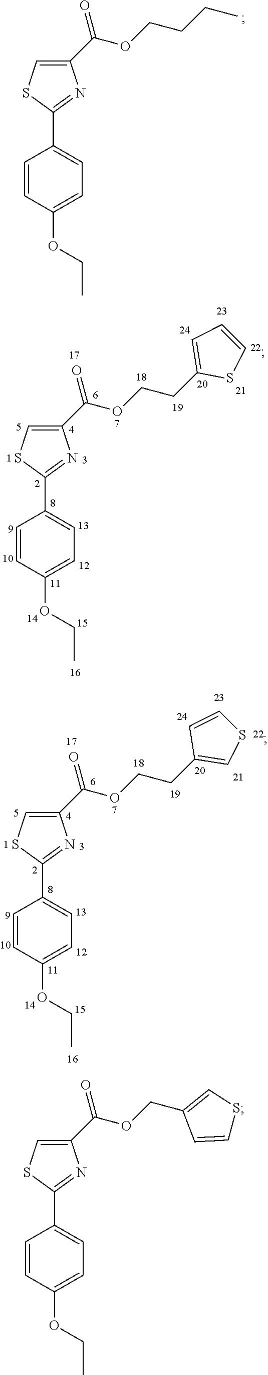

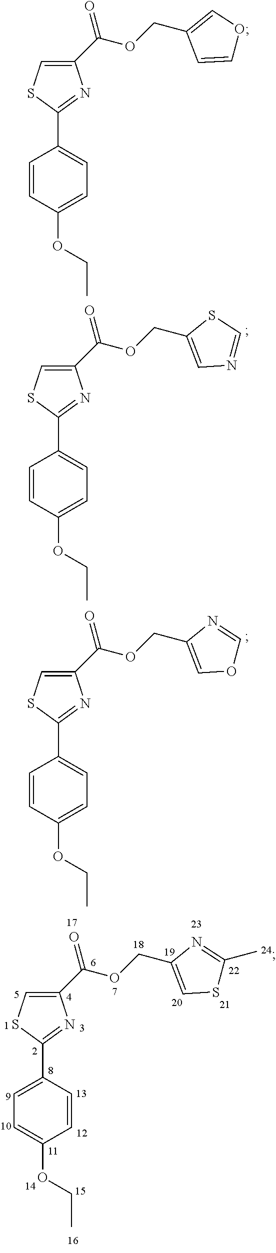

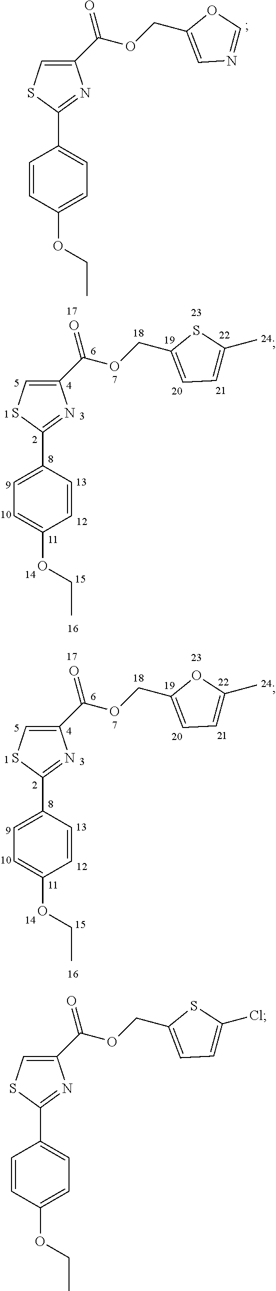

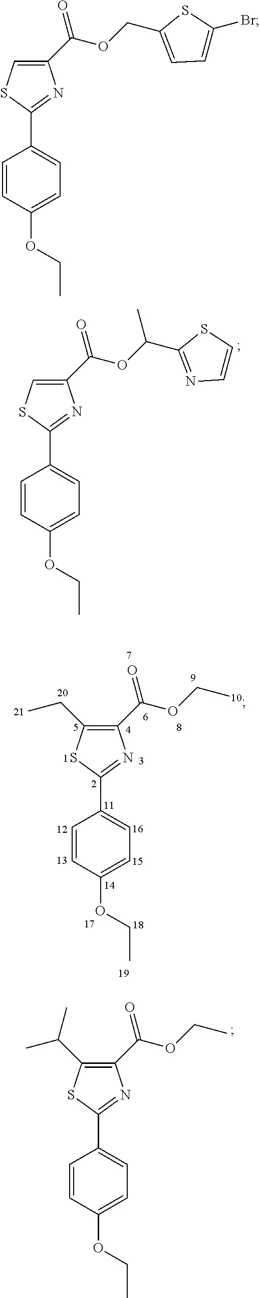

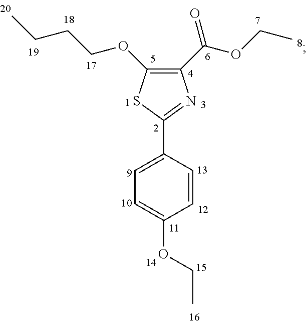 [SMILES: CCCCOc1sc(-c2ccc(OCC)cc2)nc1C(=O)OCC]

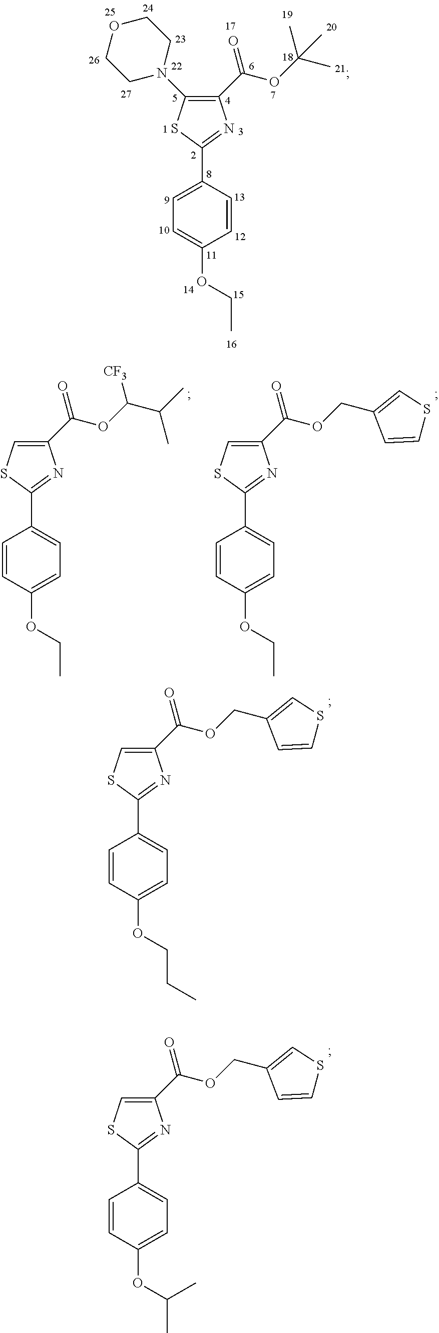

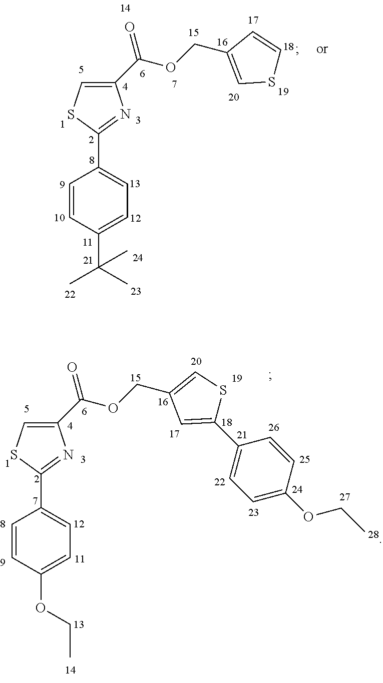 [SMILES: CC(C)(C)c1ccc(-c2nc(C(=O)OCc3ccsc3)cs2)cc1 ; CCOc1ccc(-c2cc(COC(=O)c3csc(-c4ccc(OCC)cc4)n3)cs2)cc1]

33. A pharmaceutical composition comprising a compound as defined in any one of claims 13 to 32 and a pharmaceutically acceptable excipient.

34. The compound of any one of claims 13 to 32 or the pharmaceutical composition of claim 33 for use in treating or preventing an ATGL-mediated disease or disorder.

35. The compound of any one of claims 13 to 32 or the pharmaceutical composition of claim 33 for use in treating or preventing a disease or disorder selected from a lipid metabolism disorder, obesity, non-alcoholic fatty liver disease, type 2 diabetes, insulin resistance, glucose intolerance, hypertriglyceridemia, metabolic syndrome, cardiac and skeletal muscle steatosis, congenital generalized lipodystrophy, familial partial lipodystrophy, acquired lipodystrophy syndrome, atherosclerosis, and heart failure.

36. In vitro use of a compound as defined in any one of claims 1 to 32 as an ATGL inhibitor.
